(19) United States
(12) Patent Application Publication
(10) Pub. No.: US 2026/0137729 A1
(43) Pub. Date: May 21, 2026

(54) RECOMBINANT BACTERIA EXPRESSING PHENYLALANINE AMMONIA LYASE, PHENYLALANINE TRANSPORTER AND L-AMINO ACID DEAMINASE FOR REDUCING HYPERPHENYLALANINEMIA

(71) Applicant: Synlogic Operating Company, Inc., Winchester, MA (US)

(72) Inventors: Aoife Brennan, Dover, MA (US); Caroline Kurtz, Sudbury, MA (US); Marja Puurunen, Cambridge, MA (US); Neal Sondheimer, Toronto (CA)

(73) Assignee: Synlogic Operating Company, Inc., Winchester, MA (US)

(21) Appl. No.: 19/121,180

(22) PCT Filed: Oct. 17, 2023
(86) PCT No.: PCT/US2023/077060

§ 371 Date: Apr. 15, 2025

Related U.S. Application Data
(60) Provisional application No. 63/379,826, filed on Oct. 17, 2022.
(60) Provisional application No. 63/484,390, filed on Feb. 10, 2023.
(60) Provisional application No. 63/452,822, filed on Mar. 17, 2023.
(60) Provisional application No. 63/468,419, filed on May 23, 2023.

(51) Int. Cl. A61K 35/74; A61K 31/519; A61K 38/00; A61K 47/02; A61K 47/26; A61K 47/46; A61P 3/00; C07K 14/245; C12N 1/20; C12N 9/06; C12N 9/88; C12R 1/19

(52) U.S. Cl. CPC A61K 35/74; A61K 31/519; A61K 47/02; A61K 47/26; A61K 47/46; A61P 3/00; C07K 14/245; C12N 1/20; C12N 9/0022; C12N 9/88; A61K 38/00; C12R 2001/19; C12Y 104/03002; C12Y 403/01024

(57) ABSTRACT

Methods of modulating and treating diseases associated with hyperphenylalaniemia are disclosed.

### RELATED APPLICATIONS

The instant application claims priority to U.S. Provisional Application No. 63/379,826, filed on Oct. 17, 2022; U.S. Provisional Application No. 63/484,390, filed on Feb. 10, 2023; U.S. Provisional Application No. 63/452,822, filed on Mar. 17, 2023; and U.S. Provisional Application No. 63/468,419, filed on May 23, 2023. The entire contents of each of the foregoing applications are expressly incorporated by reference herein.

### SEQUENCE LISTING

The application contains a Sequence Listing which has been submitted electronically in .XML format and is hereby incorporated by reference in its entirety. Said .XML copy, created on Oct. 16, 2023, is named “126046-09020.xml” and is 744,173 bytes in size. The sequence listing contained in this .XML file is part of the specification and is hereby incorporated by reference herein in its entirety.

### BACKGROUND

This disclosure relates to compositions and therapeutic methods for reducing hyperphenylalaninemia. In certain aspects, the disclosure relates to genetically engineered bacteria that are capable of reducing hyperphenylalaninemia in a mammal. In certain aspects, the compositions and methods disclosed herein may be used for treating diseases associated with hyperphenylalaninemia, e.g., phenylketonuria.

Phenylalanine is an essential amino acid primarily found in dietary protein. Typically, a small amount is utilized for protein synthesis, and the remainder is hydroxylated to tyrosine in an enzymatic pathway that requires phenylalanine hydroxylase (PAH) and the cofactor tetrahydrobiopterin. Hyperphenylalaninemia is a group of diseases associated with excess levels of phenylalanine, which can be toxic and cause brain damage. Primary hyperphenylalaninemia is caused by deficiencies in PAH activity that result from mutations in the PAH gene and/or a block in cofactor metabolism.

Phenylketonuria (PKU) is a severe form of hyperphenylalaninemia caused by mutations in the PAH gene. PKU is an autosomal recessive genetic disease that ranks as the most common inborn error of metabolism worldwide. The worldwide prevalence of the disease is 0.03-3.81 per 10,000 newborns with heterogeneity among countries and regions. Shoraka et al. 2020; Foreman et al. 2021). The disease affects approximately 13,000 patients in the United States. More than 500 mutations associated with the disease are recorded in the mutation database for PAH (Williams, Barua, and Andersen 2008; Blau et al. 2011). A buildup of phenylalanine (Phe) in the blood can cause profound damage to the central nervous system in children and adults. Untreated, the disease results in severe neurological complications, including irreversible loss of cognitive capacity and parkinsonism (Anikster et al. 2017; Blau et al. 2018). Treatment for PKU currently involves complete exclusion of phenylalanine from the diet. Most natural sources of protein contain phenylalanine which is an essential amino acid and necessary for growth. In patients with PKU, this means that they rely on medical foods and phe-free protein supplements together with amino acid supplements to provide just enough phenylalanine for growth. This diet is difficult for patients and has an impact on quality of life.

As discussed, current PKU therapies require substantially modified diets consisting of protein restriction. Treatment from birth generally reduces brain damage and cognitive impairment (Hoeks et al., 2009; Sarkissian et al., 1999). However, the protein-restricted diet must be carefully monitored, and essential amino acids as well as vitamins must be supplemented in the diet. Furthermore, access to low protein foods is a challenge as they are more costly than their higher protein, nonmodified counterparts (Vockley et al., 2014).

In children with PKU, growth impairment is common on a low-phenylalanine diet (Dobbelaere et al., 2003). In adulthood, new problems such as osteoporosis, maternal PKU, and vitamin deficiencies may occur (Hoeks et al., 2009). Excess levels of phenylalanine in the blood, which can freely penetrate the blood-brain barrier, can also lead to neurological impairment, behavioral problems (e.g., irritability, fatigue), and/or physical symptoms (e.g., convulsions, skin rashes, musty body odor). International guidelines recommend lifelong dietary phenylalanine restriction, which is widely regarded as difficult and unrealistic (Sarkissian et al., 1999), and “continued efforts are needed to overcome the biggest challenge to living with PKU—lifelong adherence to the low-phe diet” (Macleod et al., 2010).

In a subset of patients with residual PAH activity, oral administration of the cofactor tetrahydrobiopterin (also referred to as THB, BH4, Kuvan, or sapropterin) may be used together with dietary restriction to lower blood phenylalanine levels. However, cofactor therapy is costly and only suitable for mild forms of phenylketonuria. Additionally, the side effects of Kuvan can include gastritis and severe allergic reactions (e.g., wheezing, lightheadedness, nausea, flushing of the skin).

The enzyme phenylalanine ammonia lyase (PAL) is capable of metabolizing phenylalanine to non-toxic levels of ammonia and transcinnamic acid. Unlike PAH, PAL does not require THB cofactor activity in order to metabolize phenylalanine. Studies of oral enzyme therapy using PAL have been conducted, but “human and even the animal studies were not continued because PAL was not available in sufficient amounts at reasonable cost” (Sarkissian et al., 1999). A pegylated form of recombinant PAL (PEG-PAL; PALYNZIQ) has also been developed as an injectable form of treatment. However, subjects dosed with PEG-PAL have suffered from injection site reactions and/or developed antibodies to this therapeutic enzyme. A pegylated form of recombinant PAL (PEG-PAL; PALYZIQ) has been developed as an injectable form of treatment.

Thus, there is significant unmet need for effective, reliable, and/or long-term treatment for diseases associated with hyperphenylalaninemia, including PKU. There is an unmet need for a treatment that will control blood Phe levels in patients while allowing consumption of more natural protein.

### SUMMARY

The disclosure provides therapeutic methods for reducing hyperphenylalaninemia comprising administering genetically engineered bacteria that encode and express at least one phenylalanine metabolizing enzyme (PME), e.g., phenylalanine ammonia lyase (PAL), phenylalanine hydroxylase (PAH), and/or L-amino acid deaminase (LAAD), to a subject. Exemplary bacteria are known in the art and described herein. See, e.g., PCT/US2016/032562, PCT/US2016/062369, PCT/US2018/038840, PCT/US2021/023003, PCT/US2021/063976, PCT/US2022/076648, U.S. 63/132,627, U.S. 63/120,674, and Isabella et al., Development of a synthetic live bacterial therapeutic for the human metabolic disease phenylketonuria, Nature Biotechnology (2018), the contents of each of which are hereby incorporated by reference in their entireties.

In one aspect, the disclosure provides a method of reducing phenylalanine in a subject, comprising administering to the subject a genetically engineered bacterium comprising: a. one or more heterologous gene(s) encoding a phenylalanine ammonia lyase (PAL), b. one or more heterologous gene(s) encoding a phenylalanine transporter, and c. one or more heterologous gene(s) encoding a L-amino acid deaminase (LAAD), wherein the subject achieves a reduction in a phenylalanine level after administration as compared to a baseline level of phenylalanine in the subject before administration.

In another aspect, the disclosure provides a method of reducing hyperphenylalaninemia in a subject, comprising administering to the subject a genetically engineered bacterium comprising: a. one or more heterologous gene(s) encoding a phenylalanine ammonia lyase (PAL), b. one or more heterologous gene(s) encoding a phenylalanine transporter, and c. one or more heterologous gene(s) encoding a L-amino acid deaminase (LAAD), wherein the subject achieves a reduction in a phenylalanine level after administration as compared to a baseline level of phenylalanine in the subject before administration.

In another aspect, the disclosure provides a method of treating phenylketonuria in a subject, comprising administering to the subject a genetically engineered bacterium comprising: a. one or more heterologous gene(s) encoding a phenylalanine ammonia lyase (PAL), b. one or more heterologous gene(s) encoding a phenylalanine transporter, and c. one or more heterologous gene(s) encoding a L-amino acid deaminase (LAAD), wherein the subject achieves a reduction in a phenylalanine level after administration as compared to a baseline level of phenylalanine in the subject before administration.

In some embodiments, the genetically engineered bacterium comprises: one or more heterologous genes encoding a phenylalanine ammonia lyase (PAL) operably linked to an IPTG inducible promoter, wherein the one or more PAL genes encode a polypeptide that has at least 90%, 91%, 92%, 93%, 94%, 95%, 96%, 97%, 98%, or 99% identity to, comprises, or consists of any one of SEQ ID NO: 500 or 503, one or more heterologous genes encoding a phenylalanine transporter operably linked to an IPTG inducible promoter, wherein the one or more phenylalanine transporter genes encode a polypeptide that has at least 90%, 91%, 92%, 93%, 94%, 95%, 96%, 97%, 98%, or 99% identity to, comprises, or consists of any one of SEQ ID NO: 509, one or more heterologous genes encoding a L-amino acid deaminase (LAAD) operably linked to an arabinose-inducible promoter, wherein the one or more LAAD genes encode a polypeptide that has at least 90%, 91%, 92%, 93%, 94%, 95%, 96%, 97%, 98%, or 99% identity to, comprises, or consists of any one of SEQ ID NO: 510, a phage deletion, a ΔdapA auxotrophy, and optionally a deletion of an endogenous pks island (also referred to herein as “colibactin island”).

In some embodiments, the genetically engineered bacterium comprises: four copies of a heterologous gene encoding a phenylalanine ammonia lyase (PAL) operably linked to an IPTG inducible promoter, wherein the PAL genes encode a polypeptide that has at least 90%, 91%, 92%, 93%, 94%, 95%, 96%, 97%, 98%, or 99% identity to, comprises, or consists of any one of SEQ ID NO: 500 or 503, wherein the four copies of the PAL gene are integrated into the bacterial chromosome, one copy of a heterologous gene encoding a phenylalanine transporter operably linked to an IPTG inducible promoter, wherein the phenylalanine transporter gene encodes a polypeptide that has at least 90%, 91%, 92%, 93%, 94%, 95%, 96%, 97%, 98%, or 99% identity to, comprises, or consists of any one of SEQ ID NO: 509, wherein the phenylalanine transporter gene is integrated into the bacterial chromosome, one copy of a heterologous gene encoding a L-amino acid deaminase (LAAD) operably linked to an arabinose-inducible promoter, wherein the LAAD gene encodes a polypeptide that has at least 90%, 91%, 92%, 93%, 94%, 95%, 96%, 97%, 98%, or 99% identity to, comprises, or consists of any one of SEQ ID NO: 510, wherein the LAAD gene is integrated into the bacterial chromosome, a phage deletion, a ΔdapA auxotrophy, and optionally a deletion of an endogenous pks island.

In some embodiments, the phenylalanine level is a blood phenylalanine level. In some embodiments, the phenylalanine level is a plasma phenylalanine level.

In some embodiments, the subject achieves an improvement in at least one symptom after the administering, wherein the symptom is irritability, fatigue, convulsions, skin rashes, attention deficit, executive dysfunction, mood disorder and/or musty body odor.

In some embodiments, the subject achieves at least a 5%, at least a 10%, at least a 15%, at least a 20%, at least a 25%, at least a 30%, at least a 35%, at least a 40%, at least a 45%, at least a 50%, at least a 55%, or at least a 60% reduction in the phenylalanine level after administration as compared to the baseline level in the subject before administration. In some embodiments, the subject achieves at least a 20% reduction in phenylalanine level after administration as compared to the baseline level in the subject before administration.

In some embodiments, the subject achieves an increase in a level of trans-cinnamic acid (TCA) level after administration as compared to a baseline level of TCA in the subject before administration.

In some embodiments, the subject is capable of consuming at least about 10%, at least about 20%, at least about 25%, at least about 30%, at least about 40%, at least about 50%, at least about 60%, at least about 70%, at least about 75%, at least about 80%, at least about 85%, at least about 90%, at least about 95%, or more protein while maintaining or lowering blood or plasma phenylalanine as compared to before administration of the genetically engineered bacterium. In some embodiments, the subject is capable of consuming at least about 2-fold, at least about 3-fold, at least about 4-fold or more protein while maintaining or lowering blood or plasma phenylalanine as compared to before administration of the genetically engineered bacterium.

In some embodiments, the subject is capable of consuming at least 1 g, at least 2 g, at least 3 g, at least 4 g, at least 5 g, at least 6 g, at least 7 g, at least 8 g, at least 9 g, or at least 10 g more protein while maintaining or lowering blood or plasma phenylalanine as compared to before administration of the genetically engineered bacterium. In some embodiments, the subject is capable of consuming at least 10 g, at least 11 g, at least 12 g, at least 13 g, at least 14 g, at least 15 g, at least 16 g, at least 17 g, at least 18 g, at least 19 g, or at least 20 g more protein while maintaining or lowering blood or plasma phenylalanine as compared to before administration of the genetically engineered bacterium.

In some embodiments, the genetically engineered bacterium comprises: a. one or more heterologous gene(s) encoding a phenylalanine ammonia lyase (PAL), operably linked to a promoter that is induced under low-oxygen or anaerobic conditions, b. one or more heterologous gene(s) encoding a phenylalanine transporter, operably linked to a promoter that is induced under low-oxygen or anaerobic conditions, and c. one or more heterologous gene(s) encoding a L-amino acid deaminase (LAAD), operably linked to an arabinose-inducible promoter.

In some embodiments, the genetically engineered bacterium comprises: a. one or more heterologous gene(s) encoding a phenylalanine ammonia lyase (PAL), operably linked to an IPTG inducible promoter, b. one or more heterologous gene(s) encoding a phenylalanine transporter, operably linked to an IPTG inducible promoter, and c. one or more heterologous gene(s) encoding a L-amino acid deaminase (LAAD), operably linked to an arabinose-inducible promoter.

In some embodiments, the genetically engineered bacterium is administered at a dose (or dosage) of cells, e.g., bacterial cells, e.g., live cells, as determined by live cell counting.

In some embodiments, administering to the subject the genetically engineered bacterium at a dose of about 1×10¹¹, about 2×10¹¹, about 3×10¹¹, about 4×10¹¹, about 5×10¹¹, about 6×10¹¹, about 7×10¹¹, about 8×10¹¹, or about 9×10¹¹ bacterial cells, as determined by live cell counting. In some embodiments, administering to the subject the genetically engineered bacterium at a dose of about 1×10¹², about 2×10¹², about 3×10¹², about 4×10¹², about 5×10¹², about 6×10¹², about 7×10¹², about 8×10¹², or about 9×10¹² bacterial cells, as determined by live cell counting.

In some embodiments, administering to the subject the genetically engineered bacterium for a first dosing interval, a second dosing interval, and/or a third dosing interval.

In some embodiments, the first dosing interval, the second dosing interval, and/or the third dosing interval comprise(s) administering to the subject a dose of genetically engineered bacterium once per day (QD) for a set amount of days, then said dose twice per day (BID) for a set amount of days, and then said dose three times per day (TID) for a set amount of days.

In some embodiments, the first dosing interval comprises administering to the subject a one-third dose of genetically engineered bacterium once per day (QD) for a set amount of days, then a one-third dose twice per day (BID) for a set amount of days, and then a one-third dose three times per day (TID) for a set amount of days.

In some embodiments, the second dosing interval comprises administering to the subject a full dose of genetically engineered bacterium once per day (QD) for a set amount of days, then the full dose twice per day (BID) for a set amount of days, and then the full dose three times per day (TID) for a set amount of days.

In some embodiments, the third dosing interval comprises administering to the subject a full dose of genetically engineered bacterium once per day (QD) for a set amount of days, then the full dose twice per day (BID) for a set amount of days, and then the full dose three times per day (TID) for a set amount of days.

In some embodiments, the full dose of the second dosing interval and the full dose of the third dosing interval are different doses.

In some embodiments, a dose of genetically engineered bacterium is administered once per day (QD) for at least 1 day, at least 2 days, at least 3 days, at least 4 days, at least 5 days, at least 6 days, or at least 7 days in the first dosing interval, the second dosing interval, and/or the third dosing interval.

In some embodiments, a dose of the genetically engineered bacterium is administered twice per day (BID) for at least 1 day, at least 2 days, at least 3 days, at least 4 days, at least 5 days, at least 6 days, or at least 7 days in the first dosing interval, the second dosing interval, and/or the third dosing interval.

In some embodiments, a dose of the genetically engineered bacterium is administered three times per day (TID) for at least 1 day, at least 2 days, at least 3 days, at least 4 days, at least 5 days, at least 6 days, at least 7 days, or the remainder of days in the first dosing interval, the second dosing interval, and/or the third dosing interval.

In some embodiments, the first dosing interval, the second dosing interval, and/or the third dosing interval are at least 1 week, at least 2 weeks, at least 3 weeks, at least 4 weeks, at least 5 weeks, at least 6 weeks, at least 7 weeks, at least 8 weeks, at least 9 weeks, at least 10 weeks, or at least 11 weeks. In some embodiments, the first dosing interval, the second dosing interval, and/or the third dosing interval combined are at least 6 weeks, at least 7 weeks, at least 8 weeks, at least 9 weeks, at least 10 weeks, at least 11 weeks, at least 12 weeks, at least 13 weeks, at least 14 weeks, or at least 15 weeks.

In some embodiments, the dose of genetically engineered bacterium administered to the subject is: a. about 1×10¹¹ cells and/or 3×10¹¹ cells in the first dosing interval; b. about 6×10¹¹ cells in the second dosing interval; and/or c. about 1×10¹² cells in the third dosing interval.

In some embodiments, the genetically engineered bacterium administered to the subject in the first dosing interval is a one-third dose of about 1×10¹¹ cells and/or a full dose of about 3×10¹¹ cells.

In some embodiments, the first dosing interval is 21 days in length, and the genetically engineered bacterium is administered to the subject: a. at a dose of about 1×10¹¹ cells once per day for days 1-3; b. at a dose of about 1×10¹¹ cells twice per day for days 4-6; c. at a dose of about 1×10¹¹ cells three times per day for days 7-9; d. at a dose of about 3×10¹¹ cells once per day for days 10-11; e. at a dose of about 3×10¹¹ cells twice per day for days 12-14; and f. at a dose of about 3×10¹¹ cells three times per day for days 15-21, as determined by live cell counting.

In some embodiments, the second dosing interval is 21 days in length, and the genetically engineered bacterium is administered to the subject at a dose of about 6×10¹¹ cells, as determined by live cell counting: a. once per day for days 1-3; b. twice per day for days 4-7; and c. three times per day for days 8-21.

In some embodiments, the second dosing interval is at least 21 days in length, and the genetically engineered bacterium is administered to the subject at a dose of about 1×10¹² cells, as determined by live cell counting: a. once per day for days 1-3; b. twice per day for days 4-7; and c. three times per day for at least days 8-21.

In some embodiments, administering to the subject genetically engineered bacterium: a. for the first dosing interval only, b. for the first dosing interval and the second dosing interval, only, or c. for the first dosing interval, the second dosing interval, and the third dosing interval.

In some embodiments, administering to the subject a formulation of genetically engineered bacterium comprising the genetically engineered bacterium, sucralose, sodium bicarbonate, and a flavoring agent.

In some embodiments, administering to the subject a formulation of genetically engineered bacterium comprising the genetically engineered bacterium, sucralose, sodium bicarbonate, and a flavoring agent, wherein the amount of genetically engineered bacterium in the formulation is from about 0.5 gram to about 3.5 grams.

In some embodiments, administering to the subject a formulation of genetically engineered bacterium comprising the genetically engineered bacterium, sucralose, sodium bicarbonate, and a flavoring agent, wherein the amount of sucralose in the formulation is from about 0.001 grams to about 0.1 grams.

In some embodiments, administering to the subject a formulation of genetically engineered bacterium comprising the genetically engineered bacterium, sucralose, sodium bicarbonate, and a flavoring agent, wherein the amount of sodium bicarbonate in the formulation is from about 0.5 gram to about 3.5 grams.

In some embodiments, administering to the subject a formulation of genetically engineered bacterium comprising the genetically engineered bacterium, sucralose, sodium bicarbonate, and a flavoring agent, wherein the amount of flavoring agent in the formulation is from about 0.1 grams to about 1 gram.

In some embodiments, the subject has phenylketonuria. In some embodiments, the subject has phenylketonuria, classical or typical phenylketonuria, atypical phenylketonuria, mild hyperphenylalaninemia, nonphenylketonuric hyperphenylalaninemia, phenylalanine hydroxylase deficiency, cofactor deficiency, dihydropteridine reductase deficiency, 6-pyruvoyl tetrahydropterin synthase deficiency, DNAJC12 deficiency, Segawa's disease, progressive and irreversible neurological deficits, cognitive impairment, encephalopathy, epilepsy, eczema, reduced growth, microcephaly, tremor, limb spasticity, or hypopigmentation.

In some embodiments, the subject has a baseline blood Phe concentration greater of >360 μmol/L at baseline prior to administration of the genetically engineered bacterium or a formulation comprising the genetically engineered bacterium.

In some embodiments, the subject has a baseline blood Phe concentration of ≥600 μmol/L at baseline prior to administration of the genetically engineered bacterium a formulation comprising the genetically engineered bacterium.

In some embodiments, the method further comprises administering to the patient an additional blood phenylalanine (Phe) level management regimen.

In some embodiments, the additional blood Phe level management regimen comprises sapropterin or sepiapterin.

In some embodiments, the additional blood Phe level management regimen is administered to the subject at baseline prior to administration of the genetically engineered bacterium or a formulation comprising the genetically engineered bacterium.

In some embodiments, the subject is on a Phe-restricted diet.

In some embodiments, the subject achieves at least a 20% reduction in the level of phenylalanine after administration of the genetically engineered bacterium, or a formulation comprising the genetically engineered bacterium, as compared to the baseline level of phenylalanine.

In some embodiments, the subject achieves at least a 30% reduction in the phenylalanine level after administration of the genetically engineered bacterium, or a formulation comprising the genetically engineered bacterium, as compared to the baseline level of phenylalanine.

In some embodiments, the subject achieves at least a 40% reduction in the phenylalanine level after administration of the genetically engineered bacterium, or a formulation comprising the genetically engineered bacterium, as compared to the baseline level of phenylalanine.

In some embodiments, the baseline level of phenylalanine in the blood or plasma is ≥600 μmol/L prior to administration of the genetically engineered bacterium, or a formulation comprising the genetically engineered bacterium, and achieves a blood or plasma Phe level≤600 μmol/L after administration of the genetically engineered bacterium, or formulation comprising the genetically engineered bacterium.

In some embodiments, the baseline level of phenylalanine in the blood or plasma is ≥600 μmol/L prior to administration of the genetically engineered bacterium, or a formulation comprising the genetically engineered bacterium, and achieves a blood or plasma Phe level≤360 μmol/L after administration of the genetically engineered bacterium, or formulation comprising the genetically engineered bacterium.

In some embodiments, the baseline level of Phe in the blood or plasma is ≥600 μmol/L prior to administration of the genetically engineered bacterium, or formulation comprising the genetically engineered bacterium, and achieves a blood or plasma Phe level≤240 μmol/L after administration of the genetically engineered bacterium, or formulation comprising the genetically engineered bacterium.

In some embodiments, the baseline level of Phe in the blood or plasma is >360 μmol/L prior to administration of the genetically engineered bacterium, or formulation comprising the genetically engineered bacterium, and achieves a blood or plasma Phe level≤360 μmol/L after administration of the genetically engineered bacterium, or formulation comprising the genetically engineered bacterium.

In some embodiments, the baseline level of Phe in blood or plasma is >360 μmol/L prior to administration of the genetically engineered bacterium, or formulation comprising the genetically engineered bacterium, and achieves a blood or plasma Phe level≤240 μmol/L after administration of the genetically engineered bacterium, or formulation comprising the genetically engineered bacterium.

In some embodiments, the method further comprises measuring the baseline level of phenylalanine in blood or plasma of the subject, and selecting the subject for treatment when the baseline level is equal to or greater than 600 μmol/L, 360 μmol/L μmol/L or 240 μmol/L.

In some embodiments, the method further comprises measuring the phenylalanine level in the blood or plasma of the subject post administration, wherein a decrease of at least 20% as compared to the baseline level indicates that the treatment is effective.

In some embodiments, (i) measuring the baseline level of phenylalanine in blood or plasma of the subject, and selecting the subject for treatment when the baseline level is equal to or greater than 600 μmol/L, 360 μmol/L μmol/L or 240 μmol/L: (ii) administering the genetically engineered bacterium to the subject for a first dosing interval of 21 days: a. at a dose of about 1×10¹¹ cells once per day for days 1-3; b. at a dose of about 1×10¹¹ cells twice per day for days 4-6; c. at a dose of about 1×10¹¹ cells three times per day for days 7-9; d. at a dose of about 3×10¹¹ cells once per day for days 10-11; e. at a dose of about 3×10¹¹ cells twice per day for days 12-14; and f. at a dose of about 3×10¹¹ cells three times per day for days 15-21, as determined by live cell counting; (iii) measuring the phenylalanine level in the blood or plasma of the subject after day 21 of administration; (iv) determining whether there was at least a 20% reduction in the phenylalanine level in the blood or plasma of the subject after day 21 of administration in step (iii) with the baseline level of phenylalanine in step (i); (v) selecting the subject for further treatment with the genetically engineered bacterium once at least a 20% reduction in the phenylalanine level in the blood or plasma of the subject after administration as compared to the baseline level is achieved, and (vi) optionally further treating the patient with the genetically engineered bacterium.

In some embodiments, the method further comprises: (vii) administering the genetically engineered bacterium to the subject for a second dosing interval of 21 days at a dose of about 6×10¹¹ cells, as determined by live cell counting: a. once per day for days 1-3; b. twice per day for days 4-7; and c. three times per day for days 8-21; (viii) measuring the phenylalanine level in the blood or plasma of the subject after day 21 of administration; (ix) determining whether there was at least a 20% reduction in the phenylalanine level in the blood or plasma of the subject after day 21 of administration in step (vii) with the baseline level of phenylalanine in step (i); (x) selecting the subject for further treatment with the genetically engineered bacterium once at least a 20% reduction in the phenylalanine level in the blood or plasma of the subject after administration as compared to the baseline level is achieved, and (xi) optionally further treating the patient with the genetically engineered bacterium.

In some embodiments, the method further comprises: (xii) administering the genetically engineered bacterium to the subject for a third dosing interval of at least 21 days in length at a dose of about 1×10¹² cells, as determined by live cell counting: a. once per day for days 1-3; b. twice per day for days 4-7; and c. three times per day for at least days 8-21; (xiii) measuring the phenylalanine level in the blood or plasma of the subject after day 21 of administration; (xiv) determining whether there was at least a 20% reduction in the phenylalanine level in the blood or plasma of the subject after day 21 of administration in step (xiii) with the baseline level of phenylalanine in step (i); (xv) selecting the subject for further treatment with the genetically engineered bacterium once at least a 20% reduction in the phenylalanine level in the blood or plasma of the subject after administration as compared to the baseline level is achieved, and (xvi) optionally further treating the patient with the genetically engineered bacterium.

In some embodiments, selecting the subject for further treatment with the genetically engineered bacterium once at least a 25% or at least a 30% reduction in the phenylalanine level in the blood or plasma of the subject after administration as compared to the baseline level is achieved.

In some embodiments, the disclosure provides a method of reducing phenylalanine in a subject, comprising administering to the subject a genetically engineered bacterium comprising: one or more heterologous gene(s) encoding a phenylalanine ammonia lyase (PAL), one or more heterologous gene(s) encoding a phenylalanine transporter, one or more heterologous gene(s) encoding a L-amino acid deaminase (LAAD), wherein the subject achieves a reduction in phenylalanine levels after administration as compared to baseline levels in the subject before administration. In some embodiments, the phenylalanine levels are blood phenylalanine levels.

In some embodiments, the disclosure provides a method of reducing hyperphenylalaninemia in a subject, comprising administering to the subject a genetically engineered bacterium comprising: one or more heterologous gene(s) encoding a phenylalanine ammonia lyase (PAL), one or more heterologous gene(s) encoding a phenylalanine transporter, one or more heterologous gene(s) encoding a L-amino acid deaminase (LAAD), wherein the subject achieves an improvement in at least one symptom associated with hyperphenylalaninemia after administration as compared to baseline levels in the subject before administration.

In some embodiments, the disclosure provides a method of treating phenylketonuria in a subject, comprising administering to the subject a genetically engineered bacterium comprising: one or more heterologous gene(s) encoding a phenylalanine ammonia lyase (PAL), one or more heterologous gene(s) encoding a phenylalanine transporter, one or more heterologous gene(s) encoding a L-amino acid deaminase (LAAD), wherein the subject achieves an improvement in at least one symptom associated with phenylketonuria after administration as compared to baseline levels in the subject before administration. In some embodiments, the subject achieves at least a 5%, at least a 10%, at least a 15%, at least a 20%, at least a 25%, at least a 30%, at least a 35%, at least a 40%, at least a 45%, at least a 50%, at least a 55%, at least a 60%, at least a 70%, at least a 80%, at least a 90% or at least a 95% reduction in blood phenylalanine levels after administration as compared to baseline levels in the subject before administration.

In some embodiments, the subject achieves at least a 20% reduction in phenylalanine levels after administration as compared to baseline levels in the subject before administration.

In some embodiments, reduction of plasma phenylalanine levels after administration as compared to baseline levels in the subject before administration, are measured at day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more after administration, e.g., at day 7 or at day 14 after administration.

In some embodiments, the subject achieves at least a 20% reduction in plasma phenylalanine levels (e.g., μM plasma phenylalanine levels) after administration as compared to baseline levels in the subject before administration, e.g., as measured at day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more after administration.

In some embodiments, the subject achieves at least a 20% reduction in plasma phenylalanine levels (e.g., μM plasma phenylalanine levels) at day 7 after administration as compared to baseline levels in the subject before administration.

In some embodiments, the subject achieves at least a 20% reduction in plasma phenylalanine levels (e.g., μM plasma phenylalanine levels) at day 14 after administration as compared to baseline levels in the subject before administration.

In some embodiments, a subject is considered a “responder” if the subject achieves at least a 20% reduction in plasma phenylalanine levels (e.g., μM plasma phenylalanine levels) at day 7 or day 14 after administration as compared to baseline levels in the subject before administration.

In some embodiments, the subject achieves at least 150 μmol/L, at least 175 μmol/L, at least 200 μmol/L, at least 225 μmol/L, at least 250 μmol/L, at least 275 μmol/L, at least 300 μmol/L, at least 325 μmol/L, at least 350 μmol/L or more reduction in phenylalanine levels (e.g., μM plasma phenylalanine levels) after administration as compared to baseline levels in the subject before administration, e.g., as measured at day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more after administration.

In some embodiments, the subject achieves an increase in t-cinnamic acid (TCA) levels after administration as compared to baseline levels in the subject before administration.

In some embodiments, the subject is capable of consuming at least 10%, at least 20%, at least 25%, at least 30%, at least 40%, at least 50%, at least 60%, at least 70%, at least 75%, at least 80%, at least 85%, at least 90%, at least 95%, or more protein while maintaining or lowering blood phenylalanine as compared to before administration of the genetically engineered bacterium.

In some embodiments, the subject is capable of consuming at least 1 g, at least 2 g, at least 3 g, at least 4 g, at least 5 g, at least 6 g, at least 7 g, at least 8 g, at least 9 g, or at least 10 g more protein while maintaining or lowering blood phenylalanine as compared to before administration of the genetically engineered bacterium.

In some embodiments, the subject is capable of consuming at least 10 g, at least 11 g, at least 12 g, at least 13 g, at least 14 g, at least 15 g, at least 16 g, at least 17 g, at least 18 g, at least 19 g, or at least 20 g more protein while maintaining or lowering blood phenylalanine as compared to before administration of the genetically engineered bacterium.

In some embodiments, the genetically engineered bacterium comprises one or more heterologous gene(s) encoding a phenylalanine ammonia lyase (PAL), operably linked to a promoter that is induced under low-oxygen or anaerobic conditions, one or more heterologous gene(s) encoding a phenylalanine transporter, operably linked to a promoter that is induced under low-oxygen or anaerobic conditions, one or more heterologous gene(s) encoding a L-amino acid deaminase (LAAD), operably linked to an AraC inducible promoter.

In some embodiments, the genetically engineered bacterium comprises one or more heterologous gene(s) encoding a phenylalanine ammonia lyase (PAL), operably linked to an IPTG inducible promoter (e.g., Ptac) promoter, one or more heterologous gene(s) encoding a phenylalanine transporter, operably linked to an IPTG includible promoter (e.g., Ptac), and one or more heterologous gene(s) encoding a L-amino acid deaminase (LAAD), operably linked to an arabinose inducible, i.e., AraC inducible, promoter.

In some embodiments, the method comprises administering to the subject a formulation of genetically engineered bacteria comprising the genetically engineered bacteria, sucralose, sodium bicarbonate, and a flavoring agent.

In some embodiments, the method comprises administering to the subject genetically engineered bacteria at a dose of about 1×10¹¹, about 2×10¹¹, about 3×10¹¹, about 4×10¹¹, about 5×10¹¹, about 6×10¹¹, about 7×10¹¹, about 8×10¹¹, or about 9×10¹¹ cells, as determined by live cell counting. In some embodiments, the method comprises administering to the subject genetically engineered bacteria at a dose of about 1×10¹², about 2×10¹², about 3×10¹², about 4×10¹², about 5×10¹², about 6×10¹², about 7×10¹², about 8×10¹², or about 9×10¹² cells, as determined by live cell counting. In some embodiments, the amount of genetically engineered bacteria in the formulation is from about 0.5 gram to about 3.5 grams. In some embodiments, the amount of sucralose in the formulation is from about 0.001 grams to about 0.1 grams. In some embodiments, the amount of sodium bicarbonate in the formulation is from about 0.5 gram to about 3.5 grams. In some embodiments, the amount of flavoring agent in the formulation is from about 0.1 grams to about 1 gram.

In some embodiments, the subject has phenylketonuria, classical or typical phenylketonuria, atypical phenylketonuria, mild hyperphenylalaninemia, nonphenylketonuric hyperphenylalaninemia, phenylalanine hydroxylase deficiency, cofactor deficiency, dihydropteridine reductase deficiency, 6-pyruvoyl tetrahydropterin synthase deficiency, DNAJC12 deficiency, and Segawa's disease, progressive and irreversible neurological deficits, cognitive impairment, encephalopathy, epilepsy, eczema, reduced growth, microcephaly, tremor, limb spasticity, or hypopigmentation.

In some embodiments, the subject has a baseline blood Phe concentration≥360 μmol/L prior to administration of the genetically engineered bacterium or the formulation comprising the genetically engineered bacterium. In some embodiments, the subject has a baseline blood Phe concentration≥600 μmol/L prior to administration of the genetically engineered bacterium or the formulation comprising the genetically engineered bacterium.

In some embodiments, the method further comprises administering to the patient an additional blood Phe level management regimen. Non-limiting examples of Phe management regimens are described herein and include sapropterin or sepiapterin. In some embodiments, the additional blood Phe level management regimen is administered to the subject at baseline prior to administration of the genetically engineered bacterium or the formulation comprising the genetically engineered bacterium.

In some embodiments, the subject is on a Phe-restricted diet.

In some embodiments, the subject achieves at least a 20% reduction in phenylalanine levels after administration as compared to baseline levels in the subject before administration. In some embodiments, the subject achieves at least a 30% reduction in phenylalanine levels after administration as compared to baseline levels in the subject before administration.

In some embodiments, the subject has a baseline level of blood Phe≥600 mol/L prior to administration of the genetically engineered bacterium or formulation comprising the genetically engineered bacterium and achieves a blood Phe level≤600 μmol/L after administration of the genetically engineered bacterium or formulation comprising the genetically engineered bacterium. In some embodiments, the subject has a baseline level of blood Phe≥600 μmol/L prior to administration of the genetically engineered bacterium or formulation comprising the genetically engineered bacterium and achieves a blood Phe level≤360 μmol/L after administration of the genetically engineered bacterium or formulation comprising the genetically engineered bacterium. In some embodiments, the subject has a baseline level of blood Phe≥600 μmol/L prior to administration of the genetically engineered bacterium or formulation comprising the genetically engineered bacterium and achieves a blood Phe level≤240 μmol/L after administration of the genetically engineered bacterium or formulation comprising the genetically engineered bacterium. In some embodiments, the subject has a baseline level of blood Phe≥360 μmol/L prior to administration of the genetically engineered bacterium or formulation comprising the genetically engineered bacterium and achieves a blood Phe level≤360 μmol/L after administration of the genetically engineered bacterium or formulation comprising the genetically engineered bacterium. In some embodiments, the subject has a baseline level of blood Phe≥360 μmol/L prior to administration of the genetically engineered bacterium or formulation comprising the genetically engineered bacterium and achieves a blood Phe level≤240 μmol/L after administration of the genetically engineered bacterium or formulation comprising the genetically engineered bacterium.

In another aspect, the disclosure provides a genetically engineered bacterium comprising: one or more heterologous genes encoding a phenylalanine ammonia lyase (PAL) operably linked to an IPTG inducible promoter, wherein the one or more PAL genes encode a polypeptide that has at least 90%, 91%, 92%, 93%, 94%, 95%, 96%, 97%, 98%, or 99% identity to, comprises, or consists of any one of SEQ ID NO: 500 or 503, one or more heterologous genes encoding a phenylalanine transporter operably linked to an IPTG inducible promoter, wherein the one or more phenylalanine transporter genes encode a polypeptide that has at least 90%, 91%, 92%, 93%, 94%, 95%, 96%, 97%, 98%, or 99% identity to, comprises, or consists of any one of SEQ ID NO: 509, one or more heterologous genes encoding a L-amino acid deaminase (LAAD) operably linked to an arabinose-inducible promoter, wherein the one or more LAAD genes encode a polypeptide that has at least 90%, 91%, 92%, 93%, 94%, 95%, 96%, 97%, 98%, or 99% identity to, comprises, or consists of any one of SEQ ID NO: 510, a phage deletion, a ΔdapA auxotrophy, and optionally a deletion of an endogenous pks island.

In another aspect, the disclosure provides a genetically engineered bacterium which comprises: four copies of a heterologous gene encoding a phenylalanine ammonia lyase (PAL) operably linked to IPTG inducible promoter, wherein the PAL genes encode a polypeptide that has at least 90%, 91%, 92%, 93%, 94%, 95%, 96%, 97%, 98%, or 99% identity to, comprises, or consists of any one of SEQ ID NO: 500 or 503, wherein the four copies of the PAL gene are integrated into the bacterial chromosome, one copy of a heterologous gene encoding a phenylalanine transporter operably linked to an IPTG inducible promoter, wherein the phenylalanine transporter gene encodes a polypeptide that has at least 90%, 91%, 92%, 93%, 94%, 95%, 96%, 97%, 98%, or 99% identity to, comprises, or consists of any one of SEQ ID NO: 509, wherein the phenylalanine transporter gene is integrated into the bacterial chromosome, one copy of a heterologous gene encoding a L-amino acid deaminase (LAAD) operably linked to an arabinose-inducible promoter, wherein the LAAD gene encodes a polypeptide that has at least 90%, 91%, 92%, 93%, 94%, 95%, 96%, 97%, 98%, or 99% identity to, comprises, or consists of any one of SEQ ID NO: 510, wherein the LAAD gene is integrated into the bacterial chromosome, a phage deletion, and a ΔdapA auxotrophy. In some embodiments, the bacterium further comprises a deletion in an endogenous pks island.

### BRIEF DESCRIPTION OF THE FIGURES

FIG. 1 depicts an exemplary genetically engineered bacterium for reducing hyperphenylalaninemia and treating disorders characterized by hyperphenylalaninemia. “PAL3” refers to a PAL enzyme derived from Photorhabdus luminescens. FIG. 2A depicts a schematic of phenylalanine hydroxylase action in phenylketonuria (PKU). FIG. 2B depicts a schematic of PAH action. FIG. 2C depicts a schematic of PAL action. FIG. 2D depicts a schematic of LAAD, e.g., from Proteus mirabilis, action.

FIG. 3 depicts exemplary genetically engineered bacterium for reducing hyperphenylalaninemia and treating disorders characterized by hyperphenylalaninemia.

FIG. 4 depicts exemplary genetically engineered bacterium for reducing hyperphenylalaninemia and treating disorders characterized by hyperphenylalaninemia.

FIG. 5 depicts exemplary genetically engineered bacterium for reducing hyperphenylalaninemia and treating disorders characterized by hyperphenylalaninemia.

FIG. 6 depicts an exemplary genetically engineered bacterium (comprising certain features of SYNB1618 described herein) for reducing hyperphenylalaninemia and treating disorders characterized by hyperphenylalaninemia.

FIG. 7 depicts exemplary genetically engineered bacterium comprising certain features of SYNB1934 described herein) for reducing hyperphenylalaninemia and treating disorders characterized by hyperphenylalaninemia.

FIGS. 8A and 8B depict the results of a tracer study, in which subjects received a meal replacement shake followed by an oral dose of a deuterated phenylalanine, i.e., D5-phenylalanine (D5-Phe) isotopic tracer at a dose of 1 g dissolved in 100 mL of diluent and blood and urine was collected over a period of 6 hours. FIG. 8A depicts dose-dependent production of overall hippuric acid (HA) and D5-HA production during the tracer study at various doses of SYNB1618 at the end of the dosing period in healthy volunteers. FIG. 8B depicts dose-dependent TCA production during the tracer study at various doses of SYNB1934 at the end of the dosing period in healthy volunteers.

FIGS. 9A and 9B depict percent change observed in D5-TCA and D5 HA and D5-phenylalanine in the tracer study shown in FIGS. 8A and 8B. FIG. 9A depicts the relative percent change of increased D5-TCA and D5 HA production between SYNB1934 and SYNB1618 in healthy volunteers. FIG. 9B depicts the mean percent reduction in D5-phenyalanine using either placebo or SYNB1934 at two different doses at the end of the dosing period in healthy volunteers.

FIGS. 10A and 10B depict the interim results from an oral D5-Phe tracer study (N=8), conducted after overnight fast at baseline (Day −1) and on Day 14. Patients received a meal supplement shake, an oral dose of D5-Phe, and an oral dose of assigned IMP. Blood and urine samples were taken for the following 24 hours. FIG. 10A change from baseline of TCA AUC and percent baseline of D5 Phe, demonstrating a reduction in phenylalanine and an increase in TCA biomarker production relative to baseline (SYNB1618) in PKU patients. Final results are shown in FIGS. 17A and 17B. FIG. 10B depicts the reduction of D5-phenylalanine absorption in patients with phenylketonuria at Day 14 after administration of SYNB1618 relative to baseline. Patients underwent a meal challenge with a protein shake (20 g) and D5-Phe (1 g). (N=8). Final analysis is shown in FIG. 13A.

FIG. 11 depicts the amount of phenylalanine in blood at baseline, day 7 of treatment, day 14 of treatment, and day 29 after the washout period (SYNB1618) in PKU patients. N=8 data from interim analysis (final analysis shown in FIG. 15A).

FIG. 12 depicts the absolute amount change from baseline (μM) of phenylalanine in blood at day 7 of treatment, day 14 of treatment, and day 29 after the washout period (SYNB1618) at the time of the interim analysis (n=8).

FIGS. 13A and 13B depict the reduction of D5-phenylalanine absorption in PKU patients with phenylketonuria at Day 14 after administration of SYNB1618 (2e12 dose) relative to baseline prior to administration (Day −1) in SYNB1618 (FIG. 13A) and SYNB1934 (FIG. 13B). Patients underwent a meal challenge with a protein shake (20 g) (protein bound, unlabeled Phe) and D5-Phe (1 g) at Day −1 and Day 14. Both of these changes were statistically significant (the 95% confidence interval for decrease did not overlap zero). Samples were obtained over 24 hours (−15, 15, 30, 45, 60, 120, 180, 240, 360, 1440 min), on Days −1 and 14 for all patients with complete samples Arm 1 (SYNB1618—Panel A) and Arm 2 (SYNB1934—Panel B). Median values and interquartile range shown at each timepoint for Day −1 (round) and Day 14 (square).

FIG. 14A depicts the amount change from baseline (μM) of phenylalanine in blood at day 7 of treatment, day 14 of treatment, and day 29 after the washout period (SYNB1618). FIG. 14B depicts the amount change from baseline (μM) of phenylalanine in blood at day 7 of treatment, day 14 of treatment, and day 29 after the washout period (SYNB1934).

FIGS. 15A and 15B depict the percent change from baseline of phenylalanine in blood at day 7 of treatment, day 14 of treatment, and day 29 after the washout period for SYNB1618 (FIG. 15A) and SYNB1934 (FIG. 15B) in PKU patients. Values were obtained from blood draws taken before breakfast on Days −1 (baseline), 7, 14, and 29. LS mean change from baseline was calculated for days 7, 14, 29 using a mixed model of repeated measures. Estimates were performed on a log scale and back transformed to ratios for reporting. A. Arm 1, SYNB1618. B. Arm 2 SYNB1934. CI=confidence interval; LS=least squares.

FIG. 16A summarizes findings of blood lowering for “all corners” for both study arms in PKU patients at the 1×10¹² dose. Data are LS mean+/−95% CI; SYNB1618 n=10; SYNB1934 n=5; *Defined as those that completed dosing; Note: The 95% confidence interval did not cross zero for either strain.

FIG. 16B shows the percentage of phe lowering in PKU patients achieved for responders in both study arms. ¹Responder definition: ≥20% reduction vs. baseline in plasma Phe levels achieved on Day 7 or Day 14.

FIG. 16C shows findings integrating SYNB1934 and SYNB1618 in PKU patients in both study arms (n=15) along responder criteria of Phe lowering.

FIG. 16D shows the findings of a post-hoc responder analysis for arm 2 (SYNB1934), based on the responder definition of >20% Phe reduction. * Responder definition: >20% reduction vs. baseline in plasma Phe levels achieved on Day 7 or Day 14. The left panel shows the response rate. The right panel shows the level of phe lowering for all three responders.

FIGS. 17A and 17B show levels across biomarkers for SYNB1618 and SYNB1934 in PKU patients. FIG. 17A shows % change of Plasma D5-Phe at day 14. FIG. 17B shows change in AUC and AeT, respectively, of D5-TCA (left panel), and D5-HA (right panel). Study results show that Phe is consumed, and that data obtained are consistent with Phase 1 study data with healthy volunteers. The LS mean change from baseline Day −1 in D5 Phe metabolites was determined at day 14 for SYNB1618, n=10 and SYNB1934, n=5. Left panel: Plasma D5 TCA AUC change from baseline in LS mean AUC 0-24 μM*h. Right Panel: Urinary D5 HA change from baseline in amount excreted (AeT) in mmol over 6 hours. TCA=trans-cinnamic acid; HA=hippuric acid, AUC=area under the curve, AeT=total amount excreted

FIG. 18 shows individual results by patient (maximum Phe reduction, Day 7 or 14). Among responders, ranges for Phe reduction were 20%-61% and 29%-80% for SYNB1618 and SYNB1934, respectively.

FIGS. 19A-19B show study results from two participants, one dosed with SYNB1618 arm and one dosed with SYNB1934. Both were taking sapropterin (Kuvan®) at screening and continued on the same regimen throughout the study. FIG. 19A shows absolute levels of fasting Phe during treatment (day −1 through day 14) and after treatment (day 14 through day 29) for the subject dosed with SYNB1618. At day 14, percent Phe reduction relative to baseline was about 26%. Phe intake was 705 mg. FIG. 19B shows absolute levels of fasting Phe during treatment (day −1 through day 14) and after treatment (day 14 through day 29) for the subject dosed with SYNB1934. At day 14, percent Phe reduction relative to baseline was about 80%.

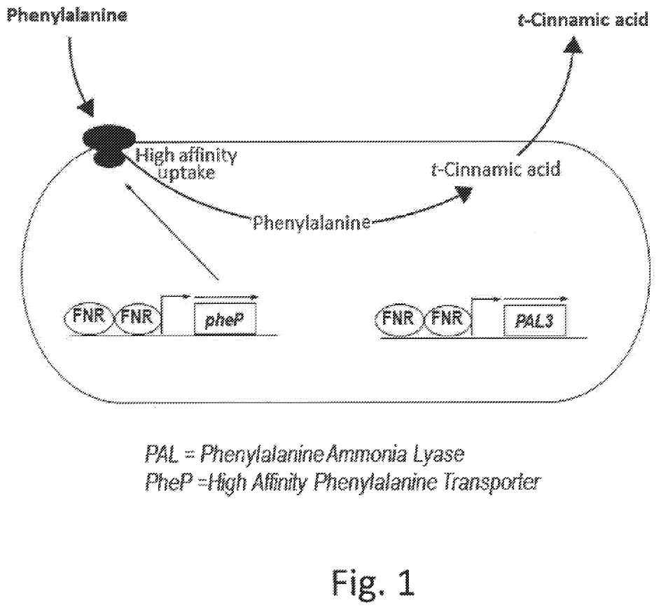

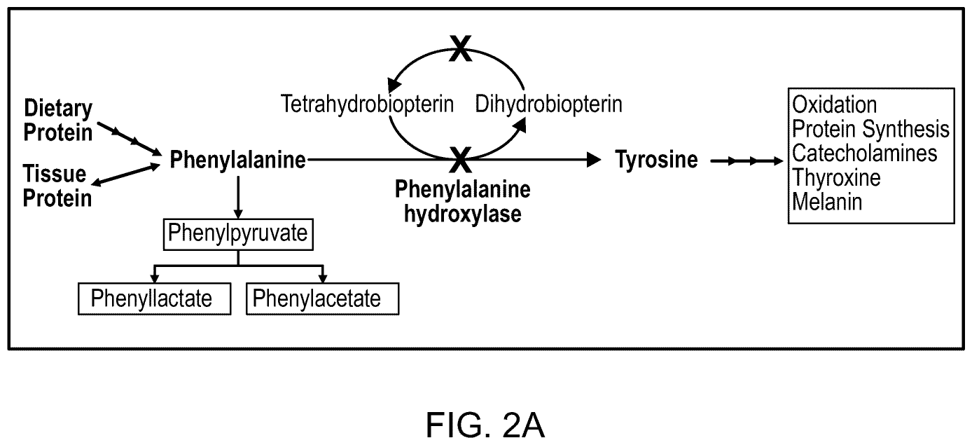

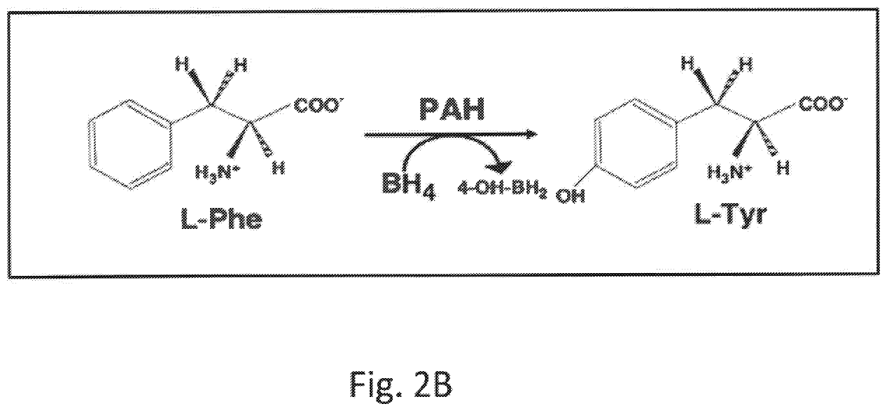

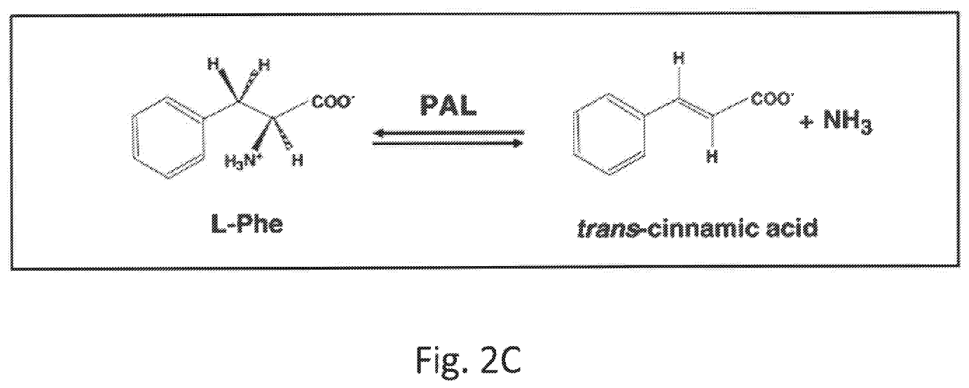

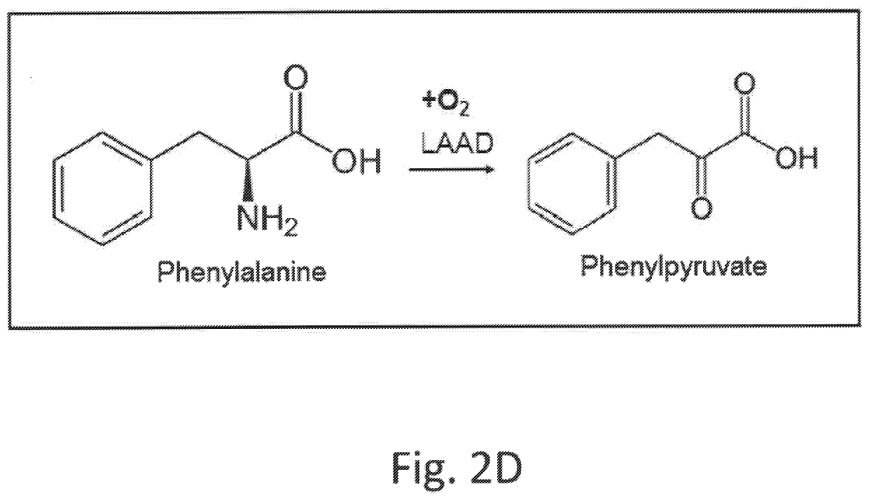

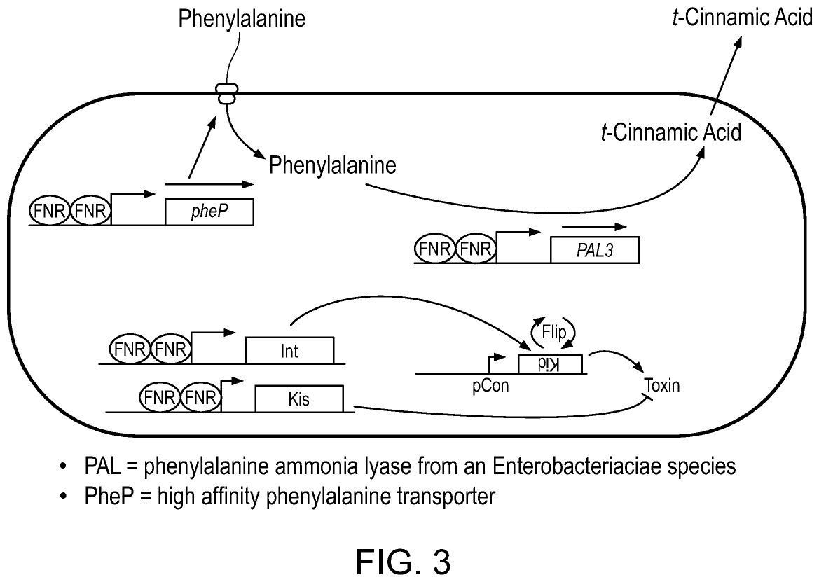

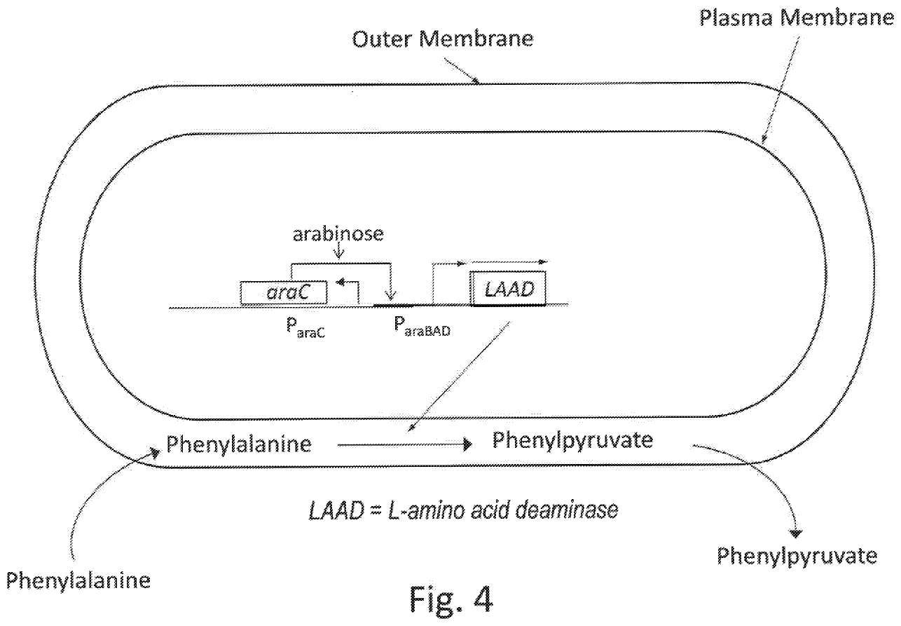

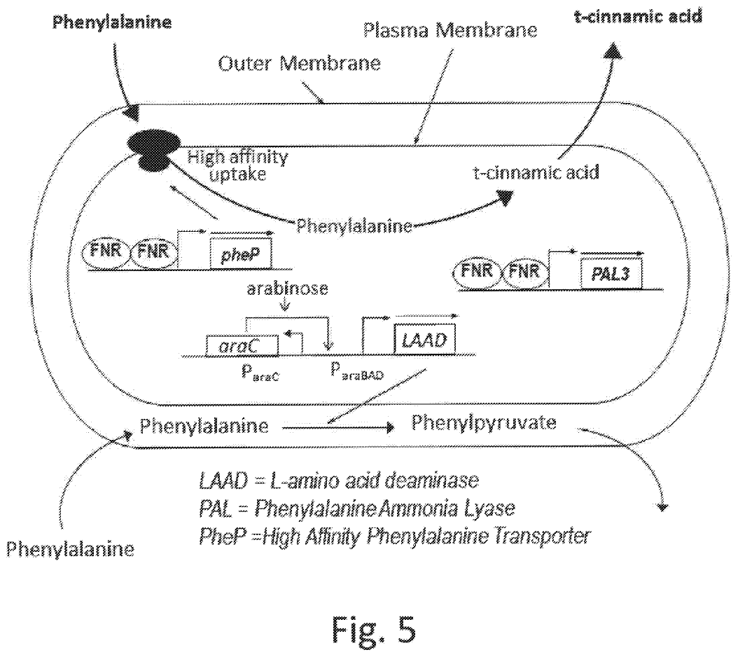

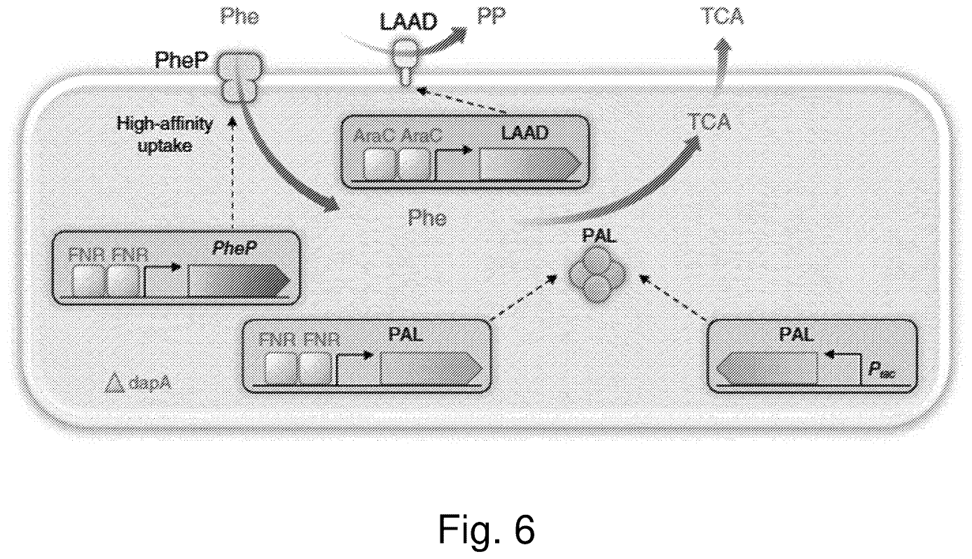

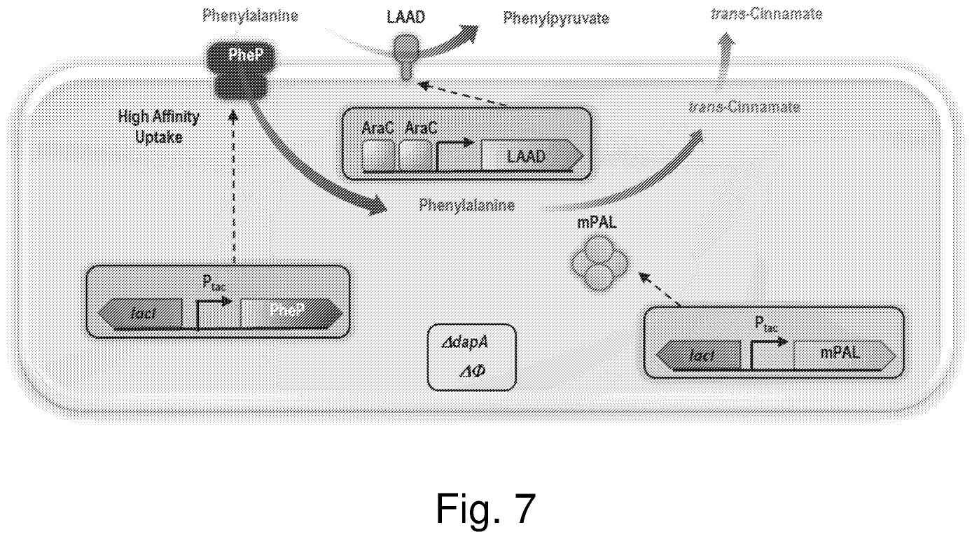

### DETAILED DESCRIPTION

The present disclosure includes genetically engineered bacteria, pharmaceutical compositions thereof, and methods of modulating and treating disorders associated with hyperphenylalaninemia. In some embodiments, the genetically engineered bacteria comprise a gene encoding non-native phenylalanine ammonia lyase (PAL) and are capable of processing and reducing phenylalanine in a mammal. Thus, the genetically engineered bacteria and pharmaceutical compositions comprising those bacteria may be used to metabolize phenylalanine in the body into non-toxic molecules in order to treat and/or prevent conditions associated with hyperphenylalaninemia, including PKU. In certain aspects, the compositions comprising the genetically engineered bacteria may be used in the methods of the disclosure to treat and/or prevent disorders associated with hyperphenylalaninemia.

### Definitions

In order that the disclosure may be more readily understood, certain terms are first defined. These definitions should be read in light of the remainder of the disclosure and as understood by a person of ordinary skill in the art. Unless defined otherwise, all technical and scientific terms used herein have the same meaning as commonly understood by a person of ordinary skill in the art. Additional definitions are set forth throughout the detailed description.

“Hyperphenylalaninemia,” “hyperphenylalaninemic,” and “excess phenylalanine” are used interchangeably herein to refer to increased or abnormally high concentrations of phenylalanine in the body. In some embodiments, a diagnostic signal of hyperphenylalaninemia is a blood phenylalanine level of at least 2 mg/dL, at least 4 mg/dL, at least 6 mg/dL, at least 8 mg/dL, at least 10 mg/dL, at least 12 mg/dL, at least 14 mg/dL, at least 16 mg/dL, at least 18 mg/dL, at least 20 mg/dL, or at least 25 mg/dL.

In some embodiments, a diagnostic signal of hyperphenylalaninemia is a blood phenylalanine level of at least >1200 μmol/L, at least 600-1200 μmol/L, or at least 360 to 600 μmol/l. In some embodiments, a diagnostic signal of hyperphenylalaninemia is a blood phenylalanine level of at least >1200 μmol/L, at least >600 μmol/L, or at least >360 μmol/l.

As used herein, diseases associated with hyperphenylalaninemia include, but are not limited to, phenylketonuria, classical or typical phenylketonuria, atypical phenylketonuria, mild hyperphenylalaninemia, nonphenylketonuric hyperphenylalaninemia, phenylalanine hydroxylase deficiency, cofactor deficiency, dihydropteridine reductase deficiency, 6-pyruvoyl tetrahydropterin synthase deficiency, DNAJC12 deficiency, and Segawa's disease. Affected individuals can suffer progressive and irreversible neurological deficits, cognitive impairment, encephalopathy, epilepsy, eczema, reduced growth, microcephaly, tremor, limb spasticity, and/or hypopigmentation (Leonard 2006). Hyperphenylalaninemia can also be secondary to other conditions, e.g., liver diseases.

PKU is usually classified according to the extent of elevated Phe levels and variable clinical outcomes, which are dependent on the genotype (van Spronsen et al. 2017; Blau and Longo 2015). This classification is based on the highest untreated blood Phe concentrations following a clinical diagnosis or at newborn screening (NBS) (van Spronsen et al. 2017), or Phe tolerance, defined as the highest Phe intake associated with blood Phe concentrations within a safe range (Table 1) (Blau et al. 2011. A simplified classification scheme is recommended: a) not requiring treatment, or b) requiring diet, BH4 or both according to the European PKU Guidelines (EPG) (van Wegberg et al. 2017).

TABLE 1
PKU classification based on enzyme activity,
blood Phe concentration and Phe tolerance

| | Mean | Mean | | |
|---|---|---|---|---|
| | enzyme | blood | Phe | Treatment |
| | activity | Phe | tolerance | recom- |
| Type | (%) | (μmol/L) | (mg/day) | mended |
| Classic PKU (HPA I) | <1 | >1,200 | <350 | Yes |
| Moderate PKU (HPA | <10 | 600 to 1200 | 350 to 400 | Yes |
| II) | | | | |
| Mild PKU (HPA III) | <10 | 360 to 600 | 400 to 600 | Yes |
| Mild HPA | 10 to 35 | 120 to 360 | >600 | No |

Abbreviations: HPA = hyperphenylalaninemia;
Phe = phenylalanine;
PKU = phenylketonuria (Blau et al. 2011; Procopio et al. 2016)

The following ICD-11 codes are used for PKU classification [PKU (ICD—11: 5C50.0)]:
Classic PKU (ICD—11: 5C50.00) with Phe blood serum levels exceeding 1,200 μmol/L, leading to the most severe outcome in untreated patients with complete enzyme deficiency (also named as HPA type I);
Nonclassical PKU (ICD—11: 5C50.01) with Phe serum concentrations ranging from 400 through 1,200 μmol/L, leading to mild to moderate severity;
Embryofetopathy due to maternal PKU (5C50.02): Maternal PKU (also named as maternal HPA) refers to developmental anomalies that may occur in the offspring of women affected by PKU;
Other specified PKU (ICD—11: 5C50.0Y): It is also referred to as mild HPA;
PKU, unspecified (ICD—11: 5C50.0Z).

The signs and symptoms of PKU vary from mild to severe. Untreated individuals may have a musty or mouse-like odor as a side effect of excess Phe in the body (Am 2014). Untreated, persistent severe PKU is characterized by irreversible intellectual disability, microcephaly, motor deficits, eczematous rash, autism, seizures, developmental problems, aberrant behavior and psychiatric symptoms (de Groot et al. 2010). Infants with PKU typically appear normal at birth. However, the coloration of the skin, hair, and eyes is different in children with PKU, due to high Phe levels interfering with production of melanin. This is caused by low levels of Tyr, whose metabolic pathway is blocked by deficiency of PAH. Another skin alteration that might occur is the presence of irritation or dermatitis. The child's behavior may be influenced as well due to augmented levels of phenethylamine, which in turn affects levels of other amines in the brain. Psychomotor function may be affected and observed to worsen progressively.

Behavioral problems were observed even in patients following a protein-restricted diet (van Spronsen et al. 2017). Symptoms associated with PKU are rarely lethal, and patients typically have a normal life span regardless of treatment status. Clinical manifestations and symptoms of PKU are summarized in Table 2.

TABLE 2
Summary of clinical manifestations
and co-morbidities in patients with PKU
Clinical Manifestations

Neurological symptoms:
Brisk reflexes
Spastic Paraparesis
Tremor
Epilepsia
Dystonia
Cerebellar ataxia
Parkinsonism
Cognitive disorders
Dysarthria
Visual loss
Headaches
Sensory changes/disorders; hand clumsiness gait disturbance
Psychiatric disorders:
Irritability; anxiety, depression, phobias, social maturity
deficits, social isolation, dementia,
Cognitive impairment:
Cognitive impairment/IQ
Apathy
Working memory disorders
Concentration changes
Other signs and disorders:
ParÆesthesias; alexia
Loss of consciousness and flaccid quadriplegia
aphasia
prosopagnosia
Behavioral changes
Walking changes/difficulties, Psychomotor slowdown
Co-morbidities among patients with PKU:
Acquired deformities of limbs
Asthma, rhinitis, Chronic obstructive pulmonary disease
Cardiovascular disorders
Oxidative stress
Kidney impairment
Bone mineral density disorder
Gastrointestinal disorder
Autoimmune diseases:
scleroderma, ulcerative colitis, Type 1 diabetes mellitus, autoimmune
hepatitis Type 2, alopecia universalis, and Grave's disease
Fetal abnormalities in maternal PKU

According to US-based treatment guidelines, the goal of PKU treatment is to maintain the blood concentrations of Phe between 120 and 360 μmol/L for all patients regardless of age. The EU-based guidelines are aligned for children under 12 years but recommend maintenance of blood Phe within the range of 120-600 μmol/L in patients with PKU aged 12 years or older. A clear consensus exists that patients with untreated blood Phe concentrations of less than 360 μmol/L do not require treatment and that treatment is required in those with Phe concentrations of more than 600 μmol/L.

Current standard treatment for patients with PKU is a stringent Phe-restricted diet, combined with amino acid mixtures supplemented with trace elements to prevent nutritional deficiencies (van Wegberg et al. 2017). This is achieved by excluding or severely curtailing protein-containing foods and providing a protein supplement that has other amino acids, but not Phe, along with frequent monitoring of blood Phe levels (Singh et al. 2014). With 1 g of protein containing about 50 mg of phenylalanine, most patients with classic PKU tolerate <500 mg Phe per day (10 g natural protein), while patients with mild to moderate PKU tolerate <1000 mg Phe per day (20 g natural protein) (Singh et al. 2014). While in the past it was thought that strict dietary control was only required in early childhood, current recommendations require lifelong dietary support with intensification during preconception and pregnancy for women (Vockley et al. 2014). Despite recommendations supporting life-long control of Phe levels, some children and most adults cannot comply due to the highly restrictive nature of the diet and other factors. This puts these patients at risk of cognitive and psychiatric disease and supports the need for novel treatment approaches. Adult patients who are no longer on a Phe-restricted diet can experience neurological complications that can improve or even reverse after reinstitution of treatment. Based on clinical experience and research data, adults and older adolescents are less adherent to a strict Phe-restricted diet than younger children, who are under their parents' supervision (Ashe et al. 2019).

Existing therapies include KUVAN is a synthetic form of BH4, a cofactor of the PAH enzyme that increases the activity level of the PAH enzyme, however, its activity is limited to the subset of patients who are BH4 responsive, and have less severe PKU. PALYNZIQ is a pegylated PAL enzyme for injection. However, administration of PALYNZIQ is not appropriate for all patients with PKU. Immune-mediated adverse reactions, development of hypersensitivity to other PEGylated injectable medicinal products, and anaphylaxis have been reported after administration of PALYNZIQ and may occur at any time during treatment. Therefore, despite the availability of these products, there remain large segments of the PKU population that have no therapeutic options available due to; nonresponsiveness to either KUVAN or PALYNZIQ therapy, age restrictions (PALYNZIQ is not available for patients <16), hypersensitivity reactions (allergic and anaphylactic reactions to PALYNZIQ), and patients who are not candidates to safely self-inject PALYNZIQ and manage the risk of anaphylaxis due to cognitive impairment associated with PKU.

“Phenylalanine ammonia lyase” and “PAL” are used to refer to a phenylalanine metabolizing enzyme (PME) that converts or processes phenylalanine to trans-cinnamic acid and ammonia. Trans-cinnamic acid has low toxicity and is converted by liver enzymes in mammals to hippuric acid, which is secreted in the urine. PAL may be substituted for the enzyme PAH to metabolize excess phenylalanine. PAL enzyme activity does not require THB cofactor activity. In some embodiments, PAL is encoded by a PAL gene derived from a prokaryotic species. In alternate embodiments, PAL is encoded by a PAL gene derived from a eukaryotic species. In some embodiments, PAL is encoded by a PAL gene derived from a bacterial species, including but not limited to, Achromobacter xylosoxidans, Pseudomonas aeruginosa, Photorhabdus luminescens, Anabaena variabilis, and Agrobacterium tumefaciens. In some embodiments, PAL is encoded by a PAL gene derived from Anabaena variabilis and referred to as “PAL1” herein (Moffitt et al., 2007). In some embodiments, PAL is encoded by a PAL gene derived from Photorhabdus luminescens and referred to as “PAL3” herein (Williams et al., 2005). In some embodiments, PAL is encoded by a PAL gene derived from a yeast species, e.g., Rhodosporidium toruloides (Gilbert et al., 1985). In some embodiments, PAL is encoded by a PAL gene derived from a plant species, e.g., Arabidopsis thaliana (Wanner et al., 1995). Any suitable nucleotide and amino acid sequences of PAL, or functional fragments thereof, may be used.

As used herein, PAL encompasses wild-type, naturally occurring PAL as well as mutant, non-naturally occurring PAL. As used herein, a “mutant PAL” or “PAL mutant” refers to a non-naturally occurring and/or synthetic PAL that has been modified, e.g., mutagenized, compared to a wild-type, naturally occurring PAL polynucleotide or polypeptide sequence. In some embodiments, the modification is a silent mutation, e.g., a change in the polynucleotide sequence without a change in the corresponding polypeptide sequence. In some embodiments, the mutant PAL exhibits increased stability and/or increased ability to metabolize phenylalanine and/or reduce hyperphenylalaninemia as compared to the wild-type PAL. In some embodiments the mutant PAL is derived from Photorhabdus luminescens PAL. In some embodiments, the mutant PAL polypeptide comprises one or more mutations at amino acid positions 92, 133, 167, 432, 470, 433, 263, 366 and/or 396 compared to a wild-type PAL, e.g., P. luminescens PAL. In some embodiments, the mutant PAL polypeptide comprises one or more mutations at amino acid positions S92, H133, I167, L432, V470, A433, A263, K366, and/or L396 compared to a wild-type PAL, e.g., P. luminescens PAL. In some embodiments, the mutant PAL polypeptide comprises one or more mutations at amino acid positions S92G, H133F, I167K, L432I, V470A, A433S, A263T, K366K (e.g., silent mutation in polynucleotide sequence), and/or L396L (e.g., silent mutation in polynucleotide sequence) compared to the positions in a wild-type PAL, e.g., P. luminescens PAL. In some embodiments, the mutant PAL polypeptide comprises S92G; H133M; I167K; L432I; V470A compared to the positions in a wild-type PAL, e.g., P. luminescens PAL. In some embodiments, the mutant PAL polypeptide comprises S92G; H133F; A433S; V470A compared to the positions in a wild-type PAL, e.g., P. luminescens PAL. In some embodiments, the mutant PAL polypeptide comprises S92G; H133F; A263T; K366K (e.g., silent mutation in polynucleotide sequence); L396L (e.g., silent mutation in polynucleotide sequence); V470A compared to the positions in a wild-type PAL, e.g., P. luminescens PAL. Any suitable nucleotide and amino acid sequences of PAL mutants, or functional fragments thereof, may be used. See, e.g., PCT/US2021/023003, PCT/US2021/063976, U.S. 63/132,627, the contents of which are incorporated by reference by their entireties herein.

“Phenylalanine hydroxylase” and “PAH” are used to refer to an enzyme that catalyzes the hydroxylation of the aromatic side chain of phenylalanine to create tyrosine in the human body in conjunction with the cofactor tetrahydrobiopterin. The human gene encoding PAH is located on the long (q) arm of chromosome 12 between positions 22 and 24.2. The amino acid sequence of PAH is highly conserved among mammals. Nucleic acid sequences for human and mammalian PAH are well known and widely available. The full-length human cDNA sequence for PAH was reported in 1985 (Kwok et al. 1985). Active fragments of PAH are also well known (e.g., Kobe et al. 1997).

“L-Aminoacid Deaminase” and “LAAD” are used to refer to an enzyme that catalyzes the stereospecific oxidative deamination of L-amino acids to generate their respective keto acids, ammonia, and hydrogen peroxide. For example, LAAD catalyzes the conversion of phenylalanine to phenylpyruvate. Multiple LAAD enzymes are known in the art, many of which are derived from bacteria, such as Proteus, Providencia, and Morganella, or venom. LAAD is characterized by fast reaction rate of phenylalanine degradation (Hou et al., Appl Microbiol Technol. 2015 October; 99(20):8391-402; “Production of phenylpyruvic acid from L-phenylalanine using an L-amino acid deaminase from Proteus mirabilis: comparison of enzymatic and whole-cell biotransformation approaches”). Most eukaryotic and prokaryotic L-amino acid deaminases are extracellular; however, Proteus species LAAD are localized to the plasma membrane (inner membrane), facing outward into the periplasmic space, in which the enzymatic activity resides. As a consequence of this localization, phenylalanine transport through the inner membrane into the cytoplasm is not required for Proteus LAAD mediated phenylalanine degradation. Phenylalanine is readily taken up through the outer membrane into the periplasm without a transporter, eliminating the need for a transporter to improve substrate availability.

In some embodiments, the genetically engineered bacteria comprise a LAAD gene derived from a bacterial species, including but not limited to, Proteus, Providencia, and Morganella bacteria. In some embodiments, the bacterial species is Proteus mirabilis. In some embodiments, the bacterial species is Proteus vulgaris. In some embodiments, the LAAD encoded by the genetically engineered bacteria is localized to the plasma membrane, facing into the periplasmic space and with the catalytic activity occurring in the periplasmic space.

“Phenylalanine metabolizing enzyme” or “PME” are used to refer to an enzyme which is able to degrade phenylalanine. Any phenylalanine metabolizing enzyme known in the art may be encoded by the genetically engineered bacteria. PMEs include, but are not limited to, phenylalanine hydroxylase (PAH), phenylalanine ammonia lyase (PAL), aminotransferase, L-amino acid deaminase (L-AAD), and phenylalanine dehydrogenases.

Reactions with phenylalanine hydroxylases, phenylalanine dehydrogenases or aminotransferases require cofactors, while L-AAD and PAL do not require any additional cofactors. In some embodiments, the PME produced by the genetically engineered bacteria is PAL. In some embodiments, the PME produced by the genetically engineered bacteria is LAAD. In some embodiments, the genetically engineered bacteria encode combinations of PMEs.

In some embodiments, the catalytic activity of the PME is dependent on oxygen levels. In some embodiments, the PME is catalytically active under microaerobic conditions. As a non-limiting example, LAAD catalytic activity is dependent on oxygen. In some embodiments, LAAD is active under low oxygen conditions, such as microaerobic conditions. In some embodiments, of the invention, the PME functions at very low levels of oxygen or in the absence of oxygen, e.g., as found in the colon. As a non-limiting example, PAL activity is not dependent on the presence of oxygen.

In certain embodiments, new or improved PMEs can be identified according to methods known in the art or described herein. In some embodiments, the genetically engineered bacteria comprise a gene encoding a naturally PME isolated from a viral, prokaryotic or eukaryotic organism. In some embodiments, the PME sequence has been further modified or mutated to increase one or more specific properties of the enzyme, such as stability or catalytic activity.

“Phenylalanine metabolite” refers to a metabolite that is generated as a result of the degradation of phenylalanine. The metabolite may be generated directly from phenylalanine, by the enzyme using phenylalanine as a substrate, or indirectly by a different enzyme downstream in the metabolic pathway, which acts on a phenylalanine metabolite substrate. In some embodiments, phenylalanine metabolites are produced by the genetically engineered bacteria encoding a PME.

In some embodiments, the phenylalanine metabolite results directly or indirectly from PAH activity, e.g., from PAH produced by the genetically engineered bacteria. In some embodiments, the metabolite is tyrosine. In some embodiments, the phenylalanine metabolite accumulates in the blood or the urine of a PKU patient, due to defective PAH activity. Non-limiting examples of such PKU metabolites are phenylpyruvic acid and phenyl-lactic acid. Other examples include phenylacetate, phenylethylamine, and phenylacetyl glutamine.

In some embodiments, the phenylalanine metabolite results directly or indirectly from PAL action, e.g., from PAL produced by the genetically engineered bacteria. Non-limiting examples of such PAL metabolites are trans-cinnamic acid and hippuric acid. In some embodiments, the phenylalanine metabolite results directly or indirectly from LAAD action, e.g., from LAAD produced by the genetically engineered bacteria. Examples of such LAAD metabolites are phenylpyruvate and phenyllactic acid.

“Phenylalanine transporter” is used to refer to a membrane transport protein that is capable of transporting phenylalanine into bacterial cells (see, e.g., Pi et al., 1991). In Escherichia coli, the pheP gene encodes a high affinity phenylalanine-specific permease responsible for phenylalanine transport (Pi et al., 1998). In some embodiments, the phenylalanine transporter is encoded by a pheP gene derived from a bacterial species, including but not limited to, Acinetobacter calcoaceticus, Salmonella enterica, and Escherichia coli. Other phenylalanine transporters include Aageneral amino acid permease, encoded by the aroP gene, transports three aromatic amino acids, including phenylalanine, with high affinity, and is thought, together with PheP, responsible for the lion share of phenylalanine import. Additionally, a low level of phenylalanine transport activity has been traced to the activity of the LIV-I/LS system, which is a branched-chain amino acid transporter consisting of two periplasmic binding proteins, the LIV-binding protein (LIV-I system) and LS-binding protein (LS system), and membrane components, LivHMGF. In some embodiments, the phenylalanine transporter is encoded by a aroP gene derived from a bacterial species. In some embodiments, the phenylalanine transporter is encoded by LIV-binding protein and LS-binding protein and LivHMGF genes derived from a bacterial species. In some embodiments, the genetically engineered bacteria comprise more than one type of phenylalanine transporter, selected from pheP, aroP, and the LIV-I/LS system.

“Phenylalanine” and “Phe” are used to refer to an amino acid with the formula C₆H₅CH₂CH(NH₂)COOH. Phenylalanine is a precursor for tyrosine, dopamine, norepinephrine, and epinephrine. L-phenylalanine is an essential amino acid and the form of phenylalanine primarily found in dietary protein; the stereoisomer D-phenylalanine is found is lower amounts in dietary protein; DL-phenylalanine is a combination of both forms. Phenylalanine may refer to one or more of L-phenylalanine, D-phenylalanine, and DL-phenylalanine.

As used herein, “gene expression system” refers to a combination of gene(s) and regulatory element(s) that enable or regulate gene expression. A gene expression system may comprise gene(s), e.g., encoding a mutant PAL polypeptide, together with one or more promoters, terminators, enhancers, insulators, silencers and other regulatory sequences to facilitate gene expression. In some embodiments, a gene expression system may comprise a gene encoding a mutant PAL and a promoter to which it is operably linked to facilitate gene expression. In some embodiment, a gene expression system may comprise multiple genes operably linked to one or more promoters to facilitate gene expression. In some embodiments, the multiple genes may be on the same plasmid or chromosome, e.g., in cis and operably linked to the same promoter. In some embodiments, the multiple genes may be on the different plasmid(s) or chromosome(s) and operably linked to the different promoters.

“Operably linked” refers a nucleic acid sequence, e.g., a gene encoding PAL, that is joined to a regulatory region sequence in a manner which allows expression of the nucleic acid sequence, e.g., acts in cis. A regulatory region is a nucleic acid that can direct transcription of a gene of interest and may comprise promoter sequences, enhancer sequences, response elements, protein recognition sites, inducible elements, promoter control elements, protein binding sequences, 5′ and 3′ untranslated regions, transcriptional start sites, termination sequences, polyadenylation sequences, and introns.

An “inducible promoter” refers to a regulatory region that is operably linked to one or more genes, wherein expression of the gene(s) is increased in the presence of an inducer of said regulatory region.

“Exogenous environmental condition(s)” or “environmental conditions” refer to settings or circumstances under which a promoter described herein may be induced. The phrase is meant to refer to the environmental conditions external to the engineered microorganism, but endogenous or native to the host subject environment. Thus, “exogenous” and “endogenous” may be used interchangeably to refer to environmental conditions in which the environmental conditions are endogenous to a mammalian body, but external or exogenous to an intact microorganism cell. In some embodiments, the exogenous environmental conditions are specific to the gut of a mammal. In some embodiments, the exogenous environmental conditions are specific to the upper gastrointestinal tract of a mammal. In some embodiments, the exogenous environmental conditions are specific to the lower gastrointestinal tract of a mammal. In some embodiments, the exogenous environmental conditions are specific to the small intestine of a mammal. In some embodiments, the exogenous environmental conditions are low oxygen, microaerobic, or anaerobic conditions, such as the environment of the mammalian gut. In some embodiments, exogenous environmental conditions refer to the presence of molecules or metabolites that are specific to the mammalian gut in a healthy or disease-state, e.g., propionate. In some embodiments, the exogenous environmental condition is a tissue-specific or disease-specific metabolite or molecule(s). In some embodiments, the exogenous environmental condition is a low-pH environment. In some embodiments, the genetically engineered microorganism of the disclosure comprises a pH-dependent promoter. In some embodiments, the genetically engineered microorganism of the disclosure comprises an oxygen level-dependent promoter. In some aspects, bacteria have evolved transcription factors that are capable of sensing oxygen levels. Different signaling pathways may be triggered by different oxygen levels and occur with different kinetics.

As used herein, “exogenous environmental conditions” or “environmental conditions” also refer to settings or circumstances or environmental conditions external to the engineered microorganism, which relate to in vitro culture conditions of the microorganism. “Exogenous environmental conditions” may also refer to the conditions during growth, production, and manufacture of the organism. Such conditions include aerobic culture conditions, anaerobic culture conditions, low oxygen culture conditions and other conditions under set oxygen concentrations. Such conditions also include the presence of a chemical and/or nutritional inducer, such as tetracycline, arabinose, IPTG, rhamnose, and the like in the culture medium. Such conditions also include the temperatures at which the microorganisms are grown prior to in vivo administration. For example, using certain promoter systems, certain temperatures are permissive to expression of a payload, while other temperatures are non-permissive. Oxygen levels, temperature and media composition influence such exogenous environmental conditions. Such conditions affect proliferation rate, rate of induction of the PME (e.g., PAL or LAAD), rate of induction of the transporter (e.g., PheP) and/or other regulators (e.g., FNR or FNRS24Y), and overall viability and metabolic activity of the strain during strain production.

An “oxygen level-dependent promoter” or “oxygen level-dependent regulatory region” refers to a nucleic acid sequence to which one or more oxygen level-sensing transcription factors is capable of binding, wherein the binding and/or activation of the corresponding transcription factor activates downstream gene expression. Examples of oxygen level-dependent transcription factors include, but are not limited to, FNR, ANR, and DNR. Corresponding FNR-responsive promoters, ANR-responsive promoters, and DNR-responsive promoters are known in the art (see, e.g., Castiglione et al., 2009; Eiglmeier et al., 1989; Galimand et al., 1991; Hasegawa et al., 1998; Hoeren et al., 1993; Salmon et al., 2003). Non-limiting examples are shown in Table 3. In a non-limiting example, a promoter (PfnrS) was derived from the E. coli Nissle fumarate and nitrate reductase gene S (fnrS) that is known to be highly expressed under conditions of low or no environmental oxygen (Durand and Storz, 2010; Boysen et al, 2010). The PfnrS promoter is activated under anaerobic conditions by the global transcriptional regulator FNR that is naturally found in Nissle. Under anaerobic conditions, FNR forms a dimer and binds to specific sequences in the promoters of specific genes under its control, thereby activating their expression. However, under aerobic conditions, oxygen reacts with iron-sulfur clusters in FNR dimers and converts them to an inactive form. In this way, the PfnrS inducible promoter is adopted to modulate the expression of proteins or RNA. PfnrS is used interchangeably in this application as FNRS, fnrS, FNR, P-FNRS promoter and other such related designations to indicate the promoter PfnrS.

TABLE 3
Examples of transcription factors, responsive
genes, regulatory regions

| Transcription | Examples of responsive genes, promoters, |
|---|---|
| factor | and/or regulatory regions: |
| FNR | nirB, ydfZ, pdhR, focA, ndH, hlyE, narK, |
| | narX, narG, yfiD, tdcD |
| ANR | arcDABC |
| DNR | norb, norC |

Exemplary oxygen-level dependent promoters, e.g., FNR promoters, are well known in the art and exemplary FNR promoters are provided in Table 4. See, e.g., PCT/US2016/032562 and PCT/US2016/062369, the contents of which are hereby incorporated by reference.

TABLE 4
Examples of FNR-responsive regulatory region sequences

| Description | FNR-responsive regulatory region sequences |
|---|---|
| FNR-responsive | ATCCCCATCACTCTTGATGGAGATCAATTCCCCAAGCTGCTAGA |
| regulatory region | GCGTTACCTTGCCCTTAAACATTAGCAATGTCGATTTATCAGAG |
| sequences | GGCCGACAGGCTCCCACAGGAGAAAACCG |
| SEQ ID NO: 6 | |
| FNR-responsive | CTCTTGATCGTTATCAATTCCCACGCTGTTTCAGAGCGTTACCTT |
| regulatory region | GCCCTTAAACATTAGCAATGTCGATTTATCAGAGGGCCGACAG |
| sequences | GCTCCCACAGGAGAAAACCG |
| SEQ ID NO: 7 | |
| nirB1 | GTCAGCATAACACCCTGACCTCTCATTAATTGTTCATGCCGGGC |
| SEQ ID NO: 1 | GGCACTATCGTCGTCCGGCCTTTTCCTCTCTTACTCTGCTACGTA |
| | CATCTATTTCTATAAATCCGTTCAATTTGTCTGTTTTTTGCACAA |
| | ACATGAAATATCAGACAATTCCGTGACTTAAGAAAATTTATAC |
| | AAATCAGCAATATACCCCTTAAGGAGTATATAAAGGTGAATTT |
| | GATTTACATCAATAAGCGGGGTTGCTGAATCGTTAAGGTAGGC |
| | GGTAATAGAAAAGAAATCGAGGCAAAA |
| nirB2 | CGGCCCGATCGTTGAACATAGCGGTCCGCAGGCGGCACTGCTT |
| SEQ ID NO: 8 | ACAGCAAACGGTCTGTACGCTGTCGTCTTTGTGATGTGCTTCCT |
| | GTTAGGTTTCGTCAGCCGTCACCGTCAGCATAACACCCTGACCT |
| | CTCATTAATTGCTCATGCCGGACGGCACTATCGTCGTCCGGCCT |
| | TTTCCTCTCTTCCCCCGCTACGTGCATCTATTTCTATAAACCCGC |
| | TCATTTTGTCTATTTTTTGCACAAACATGAAATATCAGACAATT |
| | CCGTGACTTAAGAAAATTTATACAAATCAGCAATATACCCATTA |
| | AGGAGTATATAAAGGTGAATTTGATTTACATCAATAAGCGGGG |
| | TTGCTGAATCGTTAAGGTAGGCGGTAATAGAAAAGAAATCGAG |
| | GCAAAAatgtttgtttaactttaagaaggagatatacat |
| nirB3 | GTCAGCATAACACCCTGACCTCTCATTAATTGCTCATGCCGGAC |
| SEQ ID NO: 9 | GGCACTATCGTCGTCCGGCCTTTTCCTCTCTTCCCCCGCTACGTG |
| | CATCTATTTCTATAAACCCGCTCATTTTGTCTATTTTTTGCACAA |
| | ACATGAAATATCAGACAATTCCGTGACTTAAGAAAATTTATAC |
| | AAATCAGCAATATACCCATTAAGGAGTATATAAAGGTGAATTT |
| | GATTTACATCAATAAGCGGGGTTGCTGAATCGTTAAGGTAGGC |
| | GGTAATAGAAAAGAAATCGAGGCAAAA |
| ydfZ | ATTTCCTCTCATCCCATCCGGGGTGAGAGTCTTTTCCCCCGACTT |
| SEQ ID NO: 2 | ATGGCTCATGCATGCATCAAAAAAGATGTGAGCTTGATCAAAA |
| | ACAAAAAATATTTCACTCGACAGGAGTATTTATATTGCGCCCGT |
| | TACGTGGGCTTCGACTGTAAATCAGAAAGGAGAAAACACCT |
| nirB + RBS | GTCAGCATAACACCCTGACCTCTCATTAATTGTTCATGCCGGGC |
| SEQ ID NO: 3 | GGCACTATCGTCGTCCGGCCTTTTCCTCTCTTACTCTGCTACGTA |
| | CATCTATTTCTATAAATCCGTTCAATTTGTCTGTTTTTTGCACAA |
| | ACATGAAATATCAGACAATTCCGTGACTTAAGAAAATTTATAC |
| | AAATCAGCAATATACCCCTTAAGGAGTATATAAAGGTGAATTT |
| | GATTTACATCAATAAGCGGGGTTGCTGAATCGTTAAGGATCCC |
| | TCTAGAAATAATTTTGTTTAACTTTAAGAAGGAGATATACAT |
| ydfZ + RBS | CATTTCCTCTCATCCCATCCGGGGTGAGAGTCTTTTCCCCCGACT |
| SEQ ID NO: 4 | TATGGCTCATGCATGCATCAAAAAAGATGTGAGCTTGATCAAA |
| | AACAAAAAATATTTCACTCGACAGGAGTATTTATATTGCGCCCG |
| | GATCCCTCTAGAAATAATTTTGTTTAACTTTAAGAAGGAGATAT |
| | ACAT |
| fnrSI | AGTTGTTCTTATTGGTGGTGTTGCTTTATGGTTGCATCGTAGTAA |
| SEQ ID NO: 5 | ATGGTTGTAACAAAAGCAATTTTTCCGGCTGTCTGTATACAAAA |
| | ACGCCGTAAAGTTTGAGCGAAGTCAATAAACTCTCTACCCATTC |
| | AGGGCAATATCTCTCTTGGATCCCTCTAGAAATAATTTTGTTTA |
| | ACTTTAAGAAGGAGATATACAT |
| fnrS2 | AGTTGTTCTTATTGGTGGTGTTGCTTTATGGTTGCATCGTAGTAA |
| SEQ ID NO: 10 | ATGGTTGTAACAAAAGCAATTTTTCCGGCTGTCTGTATACAAAA |
| | ACGCCGCAAAGTTTGAGCGAAGTCAATAAACTCTCTACCCATTC |
| | AGGGCAATATCTCTCTTGGATCCAAAGTGAACTCTAGAAATAAT |
| | TTTGTTTAACTTTAAGAAGGAGATATACAT |
| nirB + crp | TCGTCTTTGTGATGTGCTTCCTGTTAGGTTTCGTCAGCCGTCACC |
| SEQ ID NO: 11 | GTCAGCATAACACCCTGACCTCTCATTAATTGCTCATGCCGGAC |
| | GGCACTATCGTCGTCCGGCCTTTTCCTCTCTTCCCCCGCTACGTG |
| | CATCTATTTCTATAAACCCGCTCATTTTGTCTATTTTTTGCACAA |
| | ACATGAAATATCAGACAATTCCGTGACTTAAGAAAATTTATAC |
| | AAATCAGCAATATACCCATTAAGGAGTATATAAAGGTGAATTT |
| | GATTTACATCAATAAGCGGGGTTGCTGAATCGTTAAGGTAGaaat |
| | gtgatctagttcacatttGCGGTAATAGAAAAGAAATCGAGGCAA |
| | AAatgtttgtttaactttaagaaggagatatacat |
| fnrS + crp | AGTTGTTCTTATTGGTGGTGTTGCTTTATGGTTGCATCGTAGTAA |
| SEQ ID NO: 12 | ATGGTTGTAACAAAAGCAATTTTTCCGGCTGTCTGTATACAAAA |
| | ACGCCGCAAAGTTTGAGCGAAGTCAATAAACTCTCTACCCATTC |
| | AGGGCAATATCTCTCaaatgtgatctagttcacattttttgttta |
| | actttaagaaggagatatacat |

In some embodiments, the bacterium disclosed herein comprises a nucleic acid sequence that is at least 80%, at least 85%, at least 90%, at least 91%, at least 92%, at least 93%, at least 94%, at least 95%, at least 96%, at least 97%, at least 98%, at least 99%, or at least 100% identical to a promoter sequence in Table 4 or a functional fragment thereof.

As used herein, the term “low oxygen” is meant to refer to a level, amount, or concentration of oxygen (O₂) that is lower than the level, amount, or concentration of oxygen that is present in the atmosphere (e.g., <21% O_{2;} <160 torr O₂₎). Thus, the term “low oxygen condition or conditions” or “low oxygen environment” refers to conditions or environments containing lower levels of oxygen than are present in the atmosphere. In some embodiments, the term “low oxygen” is meant to refer to the level, amount, or concentration of oxygen (O₂) found in a mammalian gut, e.g., lumen, stomach, small intestine, duodenum, jejunum, ileum, large intestine, cecum, colon, distal sigmoid colon, rectum, and anal canal. In some embodiments, the term “low oxygen” is meant to refer to a level, amount, or concentration of O₂ that is 0-60 mmHg O₂ (0-60 torr O₂) (e.g., 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, and 60 mmHg O₂), including any and all incremental fraction(s) thereof (e.g., 0.2 mmHg, 0.5 mmHg O₂, 0.75 mmHg O₂, 1.25 mmHg O₂, 2.175 mmHg O₂, 3.45 mmHg O₂, 3.75 mmHg O₂, 4.5 mmHg O₂, 6.8 mmHg O₂, 11.35 mmHg O2, 46.3 mmHg O₂, 58.75 mmHg, etc., which exemplary fractions are listed here for illustrative purposes and not meant to be limiting in any way). In some embodiments, “low oxygen” refers to about 60 mmHg O₂ or less (e.g., 0 to about 60 mmHg O₂). The term “low oxygen” may also refer to a range of O₂ levels, amounts, or concentrations between 0-60 mmHg O₂ (inclusive), e.g., 0-5 mmHg O₂, <1.5 mmHg O₂, 6-10 mmHg, <8 mmHg, 47-60 mmHg, etc. which listed exemplary ranges are listed here for illustrative purposes and not meant to be limiting in any way. See, for example, Albenberg et al., Gastroenterology, 147(5): 1055-1063 (2014); Bergofsky et al., J Clin. Invest., 41(11): 1971-1980 (1962); Crompton et al., J Exp. Biol., 43: 473-478 (1965); He et al., PNAS (USA), 96: 4586-4591 (1999); McKeown, Br. J. Radiol., 87:20130676 (2014) (doi: 10.1259/brj.20130676), each of which discusses the oxygen levels found in the mammalian gut of various species and each of which are incorporated by reference herewith in their entireties. In some embodiments, the term “low oxygen” is meant to refer to the level, amount, or concentration of oxygen (O₂) found in a mammalian organ or tissue other than the gut, e.g., urogenital tract, tumor tissue, etc. in which oxygen is present at a reduced level, e.g., at a hypoxic or anoxic level. In some embodiments, “low oxygen” is meant to refer to the level, amount, or concentration of oxygen (O₂) present in partially aerobic, semi aerobic, microaerobic, nanoaerobic, microoxic, hypoxic, anoxic, and/or anaerobic conditions. Summaries of the amount of oxygen present in various organs and tissues are provided in PCT/US2016/062369, the contents of which is herein incorporated by reference in its entirety. In some embodiments, the level, amount, or concentration of oxygen (O₂) is expressed as the amount of dissolved oxygen (“DO”) which refers to the level of free, non-compound oxygen (O₂) present in liquids and is typically reported in milligrams per liter (mg/L), parts per million (ppm; 1 mg/L=1 ppm), or in micromoles (umole) (1 umole O₂=0.022391 mg/L O₂). Fondriest Environmental, Inc., “Dissolved Oxygen”, Fundamentals of Environmental Measurements, 19 Nov. 2013, www.fondriest.com/environmental-measurements/parameters/water-quality/dissolved-oxygen/>. In some embodiments, the term “low oxygen” is meant to refer to a level, amount, or concentration of oxygen (O₂) that is about 6.0 mg/L DO or less, e.g., 6.0 mg/L, 5.0 mg/L, 4.0 mg/L, 3.0 mg/L, 2.0 mg/L, 1.0 mg/L, or 0 mg/L, and any fraction therein, e.g., 3.25 mg/L, 2.5 mg/L, 1.75 mg/L, 1.5 mg/L, 1.25 mg/L, 0.9 mg/L, 0.8 mg/L, 0.7 mg/L, 0.6 mg/L, 0.5 mg/L, 0.4 mg/L, 0.3 mg/L, 0.2 mg/L and 0.1 mg/L DO, which exemplary fractions are listed here for illustrative purposes and not meant to be limiting in any way. The level of oxygen in a liquid or solution may also be reported as a percentage of air saturation or as a percentage of oxygen saturation (the ratio of the concentration of dissolved oxygen (O₂) in the solution to the maximum amount of oxygen that will dissolve in the solution at a certain temperature, pressure, and salinity under stable equilibrium). Well-aerated solutions (e.g., solutions subjected to mixing and/or stirring) without oxygen producers or consumers are 100% air saturated. In some embodiments, the term “low oxygen” is meant to refer to 40% air saturation or less, e.g., 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1%, and 0% air saturation, including any and all incremental fraction(s) thereof (e.g., 30.25%, 22.70%, 15.5%, 7.7%, 5.0%, 2.8%, 2.0%, 1.65%, 1.0%, 0.9%, 0.8%, 0.75%, 0.68%, 0.5%. 0.44%, 0.3%, 0.25%, 0.2%, 0.1%, 0.08%, 0.075%, 0.058%, 0.04%, 0.032%, 0.025%, 0.01%, etc.) and any range of air saturation levels between 0-40%, inclusive (e.g., 0-5%, 0.05-0.1%, 0.1-0.2%, 0.1-0.5%, 0.5-2.0%, 0-10%, 5-10%, 10-15%, 15-20%, 20-25%, 25-30%, etc.). The exemplary fractions and ranges listed here are for illustrative purposes and not meant to be limiting in any way. In some embodiments, the term “low oxygen” is meant to refer to 9% 02 saturation or less, e.g., 9%, 8%, 7%, 6%, 5%, 4%, 3%, 2%, 1%, 0%, 02 saturation, including any and all incremental fraction(s) thereof (e.g., 6.5%, 5.0%, 2.2%, 1.7%, 1.4%, 0.9%, 0.8%, 0.75%, 0.68%, 0.5%. 0.44%, 0.3%, 0.25%, 0.2%, 0.1%, 0.08%, 0.075%, 0.058%, 0.04%. 0.032%, 0.025%, 0.01%, etc.) and any range of 02 saturation levels between 0-9%, inclusive (e.g., 0-5%, 0.05-0.1%, 0.1-0.2%, 0.1-0.5%, 0.5-2.0%, 0-8%, 5-7%, 0.3-4.2% O_{2,} etc.). The exemplary fractions and ranges listed here are for illustrative purposes and not meant to be limiting in any way.

An inducible promoter includes a regulatory region that is induced by a chemical inducer, such as isopropyl β-D-1-thiogalactopyranoside (IPTG). IPTG is an allolactose mimic known in the art and used to induce transcription of genes having lac repressor operons within their promoter regions. In bacteria, the transcriptional regulator LacI represses the expression of genes encoding proteins related to lactose metabolism in the absence of lactose. Once lactose is available, however, it is converted into allolactose, which is capable of binding LacI and thereby allosterically inhibiting the ability of LacI to bind DNA at the lac operator and, in doing so, allowing expression of downstream genes. An “IPTG-inducible promoter” refers to a nucleic acid sequence to which an allolactose/IPTG level-sensing transcription factor, e.g., the lac repressor LacI, is capable of binding. The binding of the transcription factor to the nucleic acid sequence, e.g., a promoter or promoter region comprising a lac operon, represses downstream gene expression in the absence of IPTG. Exemplary IPTG-inducible promoters are known in the art and provided in Table 5.

In some embodiments, the inducible promoter is an IPTG-inducible promoter, e.g., Ptac. In one embodiment, the IPTG-inducible promoter comprises a sequence having at least 90%, at least 91%, at least 92%, at least 93%, at least 94%, at least 95%, at least 96%, at least 97%, at least 98%, or at least 99% identity to, comprises, or consists of SEQ ID NO: 383. In some embodiments, the IPTG-inducible promoter comprises a sequence having at least 90%, at least 91%, at least 92%, at least 93%, at least 94%, at least 95%, at least 96%, at least 97%, at least 98%, or at least 99% identity to, comprises, or consists of SEQ ID NO: 381. In some embodiments, the bacterium disclosed herein further comprises a gene sequence encoding a regulator (e.g., LacI repressor), which represses the activity of the IPTG-inducible promoter in the absence of the inducer. In some embodiments, the gene sequence encodes a repressor comprising a sequence having at least 90%, at least 91%, at least 92%, at least 93%, at least 94%, at least 95%, at least 96%, at least 97%, at least 98%, or at least 99% identity to, comprises, or consists of SEQ ID NO: 16. In some embodiments, the gene sequence encoding a repressor comprises a sequence having at least 90%, at least 91%, at least 92%, at least 93%, at least 94%, at least 95%, at least 96%, at least 97%, at least 98%, or at least 99% identity to, comprises, or consists of SEQ ID NO: 15. In some embodiments, the gene sequence encoding a repressor comprises a sequence having at least 90%, at least 91%, at least 92%, at least 93%, at least 94%, at least 95%, at least 96%, at least 97%, at least 98%, or at least 99% identity to, comprises, or consists of SEQ ID NO: 379. In some embodiments, the gene sequence encoding a repressor comprises a sequence having at least 90%, at least 91%, at least 92%, at least 93%, at least 94%, at least 95%, at least 96%, at least 97%, at least 98%, or at least 99% identity to, comprises, or consists of SEQ ID NO: 380. In some embodiments, the gene sequence encoding a repressor comprises a sequence having at least 90%, at least 91%, at least 92%, at least 93%, at least 94%, at least 95%, at least 96%, at least 97%, at least 98%, or at least 99% identity to, comprises, or consists of SEQ ID NO: 17. In some embodiments, the gene sequence encoding a repressor comprises a sequence having at least 90%, at least 91%, at least 92%, at least 93%, at least 94%, at least 95%, at least 96%, at least 97%, at least 98%, or at least 99% identity to, comprises, or consists of SEQ ID NO: 384. In these embodiments, the sequence may additionally contain SEQ ID NO: 382, 332, or 333, or a sequence having at least 90%, at least 91%, at least 92%, at least 93%, at least 94%, at least 95%, at least 96%, at least 97%, at least 98%, or at least 99% identity to SEQ ID NO: 382, 332, or 333.

TABLE 5
IPTG-inducible promoter and Lacl sequences

| Description | |
|---|---|
| SEQ ID NO | Sequences |
| LacI in reverse | TCACTGCCCGCTTTCCAGTCGGGAAACCTGTCGTGCCAGCTGCATT |
| orientation | AATGAATCGGCCAACGCGCGGGGAGAGGCGGTTTGCGTATTGGGC |
| SEQ ID NO: 15 | GCCAGGGTGGTTTTTCTTTTCACCAGTGAGACTGGCAACAGCTGAT |
| | TGCCCTTCACCGCCTGGCCCTGAGAGAGTTGCAGCAAGCGGTCCA |
| | CGCTGGTTTGCCCCAGCAGGCGAAAATCCTGTTTGATGGTGGTTAA |
| | CGGCGGGATATAACATGAGCTATCTTCGGTATCGTCGTATCCCACT |
| | ACCGAGATATCCGCACCAACGCGCAGCCCGGACTCGGTAATGGCG |
| | CGCATTGCGCCCAGCGCCATCTGATCGTTGGCAACCAGCATCGCA |
| | GTGGGAACGATGCCCTCATTCAGCATTTGCATGGTTTGTTGAAAAC |
| | CGGACATGGCACTCCAGTCGCCTTCCCGTTCCGCTATCGGCTGAAT |
| | TTGATTGCGAGTGAGATATTTATGCCAGCCAGCCAGACGCAGACG |
| | CGCCGAGACAGAACTTAATGGGCCCGCTAACAGCGCGATTTGCTG |
| | GTGACCCAATGCGACCAGATGCTCCACGCCCAGTCGCGTACCGTC |
| | CTCATGGGAGAAAATAATACTGTTGATGGGTGTCTGGTCAGAGAC |
| | ATCAAGAAATAACGCCGGAACATTAGTGCAGGCAGCTTCCACAGC |
| | AATGGCATCCTGGTCATCCAGCGGATAGTTAATGATCAGCCCACTG |
| | ACGCGTTGCGCGAGAAGATTGTGCACCGCCGCTTTACAGGCTTCG |
| | ACGCCGCTTCGTTCTACCATCGACACCACCACGCTGGCACCCAGTT |
| | GATCGGCGCGAGATTTAATCGCCGCGACAATTTGCGACGGCGCGT |
| | GCAGGGCCAGACTGGAGGTGGCAACGCCAATCAGCAACGACTGTT |
| | TGCCCGCCAGTTGTTGTGCCACGCGGTTGGGAATGTAATTCAGCTC |
| | CGCCATCGCCGCTTCCACTTTTTCCCGCGTTTTCGCAGAAACGTGG |
| | CTGGCCTGGTTCACCACGCGGGAAACGGTCTGATAAGAGACACCG |
| | GCATACTCTGCGACATCGTATAACGTTACTGGTTTCAT |
| LacI | MKPVTLYDVAEYAGVSYQTVSRVVNQASHVSAKTREKVEAAMAEL |
| SEQ ID NO: 16 | NYIPNRVAQQLAGKQSLLIGVATSSLALHAPSQIVAAIKSRADQLGAS |
| | VVVSMVERSGVEACKAAVHNLLAQRVSGLIINYPLDDQDAIAVEAAC |
| | TNVPALFLDVSDQTPINSIIFSHEDGTRLGVEHLVALGHQQIALLAGPL |
| | SSVSARLRLAGWHKYLTRNQIQPIAEREGDWSAMSGFQQTMQMLNE |
| | GIVPTAMLVANDQMALGAMRAITESGLRVGADISVVGYDDTEDSSC |
| | YIPPLTTIKQDFRLLGQTSVDRLLQLSQGQAVKGNQLLPVSLVKRKTT |
| | LAPNTQTASPRALADSLMQLARQVSRLESGQ |
| PlacI (promoter | CCGGGCGCTATCATGCCATACCGCGAAAGGTTTTGCGCCATTCGAT |
| for lacI in reverse | GG |
| orientation) | |
| SEQ ID NO: 379 | |
| PlacI-RBS-lacI | TCACTGCCCGCTTTCCAGTCGGGAAACCTGTCGTGCCAGCTGCATT |
| (reverse | AATGAATCGGCCAACGCGCGGGGAGAGGCGGTTTGCGTATTGGGC |
| orientation) | GCCAGGGTGGTTTTTCTTTTCACCAGTGAGACTGGCAACAGCTGAT |
| SEQ ID NO: 380 | TGCCCTTCACCGCCTGGCCCTGAGAGAGTTGCAGCAAGCGGTCCA |
| | CGCTGGTTTGCCCCAGCAGGCGAAAATCCTGTTTGATGGTGGTTAA |
| | CGGCGGGATATAACATGAGCTATCTTCGGTATCGTCGTATCCCACT |
| | ACCGAGATATCCGCACCAACGCGCAGCCCGGACTCGGTAATGGCG |
| | CGCATTGCGCCCAGCGCCATCTGATCGTTGGCAACCAGCATCGCA |
| | GTGGGAACGATGCCCTCATTCAGCATTTGCATGGTTTGTTGAAAAC |
| | CGGACATGGCACTCCAGTCGCCTTCCCGTTCCGCTATCGGCTGAAT |
| | TTGATTGCGAGTGAGATATTTATGCCAGCCAGCCAGACGCAGACG |
| | CGCCGAGACAGAACTTAATGGGCCCGCTAACAGCGCGATTTGCTG |
| | GTGACCCAATGCGACCAGATGCTCCACGCCCAGTCGCGTACCGTC |
| | CTCATGGGAGAAAATAATACTGTTGATGGGTGTCTGGTCAGAGAC |
| | ATCAAGAAATAACGCCGGAACATTAGTGCAGGCAGCTTCCACAGC |
| | AATGGCATCCTGGTCATCCAGCGGATAGTTAATGATCAGCCCACTG |
| | ACGCGTTGCGCGAGAAGATTGTGCACCGCCGCTTTACAGGCTTCG |
| | ACGCCGCTTCGTTCTACCATCGACACCACCACGCTGGCACCCAGTT |
| | GATCGGCGCGAGATTTAATCGCCGCGACAATTTGCGACGGCGCGT |
| | GCAGGGCCAGACTGGAGGTGGCAACGCCAATCAGCAACGACTGTT |
| | TGCCCGCCAGTTGTTGTGCCACGCGGTTGGGAATGTAATTCAGCTC |
| | CGCCATCGCCGCTTCCACTTTTTCCCGCGTTTTCGCAGAAACGTGG |
| | CTGGCCTGGTTCACCACGCGGGAAACGGTCTGATAAGAGACACCG |
| | GCATACTCTGCGACATCGTATAACGTTACTGGTTTCATATTCACCA |
| | CCCTGAATTGACTCTCTTCCGGGCGCTATCATGCCATACCGCGA |
| | AAGGTTTTGCGCCATTCGATGG |
| Ptac (minimal | ttgacaattaatcatcggctcgtataatg |
| promoter for gene | |
| expression- | |
| includes −10 and | |
| −35 region) | |
| SEQ ID NO: 381 | |
| Exemplary pTac | ttgacaattaatcatcggctcgtataatgtgtggaattgtgagcgc |
| promoter | tcacaattagctgt |
| comprising -10 | |
| and −35 regions | |
| and Lac operon | |
| SEQ ID NO: 383 | |
| Exemplary spacer | CGCGCCGCTTCGTCAGGCCACATAGCTTTCTTGTTCTGATCGGAAC |
| region | GATCGTTGGCTGtg |
| SEQ ID NO: 382 | |
| Exemplary Lac | aattgtgagcgctcacaatt |
| operator | |
| SEQ ID NO: 17 | |
| Exemplary | taacaccgtgcgtgttg |
| Operator 1 | |
| SEQ ID NO: 332 | |
| Exemplary | Tacctctggcggtgata |
| Operator 2 | |
| SEQ ID NO: 333 | |
| Exemplary LacI | ATTCACCACCCTGAATTGACTCTCTT |
| RBS (reverse | |
| orientation) | |
| SEQ ID NO: 384 | |

In some embodiments, the bacterial cells comprise endogenous gene(s) encoding the IPTG sensing transcriptional regulator, LacI. In some embodiments, the lacI gene is heterologous or non-native. In some embodiments, the gene encoding the IPTG level-sensing transcriptional regulator, e.g., LacI, is present on a plasmid. In some embodiments, the gene encoding the IPTG level-sensing transcriptional regulator, e.g., LacI, and the gene encoding the PME or phenylalanine transporter are present on different plasmids. In some embodiments, the gene encoding the IPTG level-sensing transcriptional regulator, e.g., LacI, and the gene encoding the PME or phenylalanine transporter are present on the same plasmid. In some embodiments, the gene encoding the IPTG level-sensing transcriptional regulator, e.g., LacI, is present on a chromosome. In some embodiments, the gene encoding the IPTG level-sensing transcriptional regulator, e.g., LacI, and the gene encoding the PME or phenylalanine transporter are present on different chromosomes. In some embodiments, the gene encoding the IPTG level-sensing transcriptional regulator, e.g., LacI, and the gene encoding the PME or phenylalanine transporter are present on the same chromosome, either at the same or a different insertion site. In some embodiments, expression of the transcriptional regulator is controlled by a different promoter than the promoter that controls expression of the gene encoding the PME or phenylalanine transporter, e.g., a constitutive promoter. In some embodiments, the transcriptional regulator and the phenylalanine decarboxylase or phenylalanine transporter are divergently transcribed from a promoter region.

In some embodiments, the bacterium disclosed herein comprises a nucleotide sequence that encodes a PAL sequence that is at least 80%, at least 85%, at least 90%, at least 91%, at least 92%, at least 93%, at least 94%, at least 95%, at least 96%, at least 97%, at least 98%, at least 99%, or 100% identical to a PAL amino acid sequence in Table 6 or a functional fragment thereof.

In one embodiment, the phenylalanine ammonia lyase (PAL) gene encodes a polypeptide has at least about 80% identity with SEQ ID NO: 500. Accordingly, in one embodiment, the PAL gene encodes a polypeptide has at least about 90% identity with SEQ ID NO: 500. Accordingly, in one embodiment, the PAL gene encodes a polypeptide has at least about 95% identity with SEQ ID NO: 500. Accordingly, in one embodiment, the PAL gene encodes a polypeptide has at least about 85%, 86%, 87%, 88%, 89%, 90%, 91%, 92%, 93%, 94%, 95%, 96%, 97%, 98%, or 99% identity with SEQ ID NO: 500. In another embodiment, the PAL gene encodes a polypeptide comprises SEQ ID NO: 500. In yet another embodiment, the PAL gene encodes a polypeptide consists of SEQ ID NO: 500.

In one embodiment, the phenylalanine ammonia lyase (PAL) gene encodes a polypeptide has at least about 80% identity with SEQ ID NO: 503. Accordingly, in one embodiment, the PAL gene encodes a polypeptide has at least about 90% identity with SEQ ID NO: 503. Accordingly, in one embodiment, the PAL gene encodes a polypeptide has at least about 95% identity with SEQ ID NO: 503. Accordingly, in one embodiment, the PAL gene encodes a polypeptide has at least about 85%, 86%, 87%, 88%, 89%, 90%, 91%, 92%, 93%, 94%, 95%, 96%, 97%, 98%, or 99% identity with SEQ ID NO: 503. In another embodiment, the PAL gene encodes a polypeptide comprises SEQ ID NO: 503. In yet another embodiment, the PAL gene encodes a polypeptide consists of SEQ ID NO: 503.

In some embodiments, the bacterium disclosed herein comprises a nucleotide sequence that encodes a PheP sequence that is at least 80%, at least 85%, at least 90%, at least 91%, at least 92%, at least 93%, at least 94%, at least 95%, at least 96%, at least 97%, at least 98%, at least 99%, or 100% identical to a PheP amino acid sequence in Table 16 or a functional fragment thereof.

In one embodiment, the phenylalanine transporter gene encodes a polypeptide has at least about 80% identity with SEQ ID NO: 509. Accordingly, in one embodiment, the phenylalanine transporter gene encodes a polypeptide has at least about 90% identity with SEQ ID NO: 509. Accordingly, in one embodiment, the phenylalanine transporter gene encodes a polypeptide has at least about 95% identity with SEQ ID NO: 509. Accordingly, in one embodiment, the phenylalanine transporter gene encodes a polypeptide has at least about 85%, 86%, 87%, 88%, 89%, 90%, 91%, 92%, 93%, 94%, 95%, 96%, 97%, 98%, or 99% identity with SEQ ID NO: 509. In another embodiment, the phenylalanine transporter gene encodes a polypeptide comprises SEQ ID NO: 509. In yet another embodiment, the phenylalanine transporter gene encodes a polypeptide consists of SEQ ID NO: 509.

In some embodiments, the bacterium disclosed herein comprises a nucleotide sequence that encodes a LAAD sequence that is at least 80%, at least 85%, at least 90%, at least 91%, at least 92%, at least 93%, at least 94%, at least 95%, at least 96%, at least 97%, at least 98%, at least 99%, or 100% identical to a LAAD amino acid sequence in Table 16 or a functional fragment thereof.

In one embodiment, the L-amino acid deaminase (LAAD) gene encodes a polypeptide has at least about 80% identity with SEQ ID NO: 510. Accordingly, in one embodiment, the LAAD gene encodes a polypeptide has at least about 90% identity with SEQ ID NO: 510. Accordingly, in one embodiment, the LAAD gene encodes a polypeptide has at least about 95% identity with SEQ ID NO: 510. Accordingly, in one embodiment, the LAAD gene encodes a polypeptide has at least about 85%, 86%, 87%, 88%, 89%, 90%, 91%, 92%, 93%, 94%, 95%, 96%, 97%, 98%, or 99% identity with SEQ ID NO: 510. In another embodiment, the LAAD gene encodes a polypeptide comprises SEQ ID NO: 510. In yet another embodiment, the LAAD gene encodes a polypeptide consists of SEQ ID NO: 510.

In some embodiments, the bacterium disclosed herein comprises a nucleic acid sequence that is at least 80%, at least 85%, at least 90%, at least 91%, at least 92%, at least 93%, at least 94%, at least 95%, at least 96%, at least 97%, at least 98%, at least 99%, or 100% identical to a promoter sequence in Table 5 or a functional fragment thereof.

As used herein, a “non-native” nucleic acid sequence refers to a nucleic acid sequence not normally present in a bacterium. e.g., an extra copy of an endogenous sequence, or a heterologous sequence such as a sequence from a different species, strain, or substrain of bacteria, or a sequence that is modified and/or mutated as compared to the unmodified sequence from bacteria of the same subtype. In some embodiments, the non-native nucleic acid sequence is a synthetic, non-naturally occurring sequence (see, e.g., Purcell et al., 2013). The non-native nucleic acid sequence may be a regulatory region, a promoter, a gene, and/or one or more genes in a gene cassette. In some embodiments, “non-native” refers to two or more nucleic acid sequences that are not found in the same relationship to each other in nature. The non-native nucleic acid sequence may be present on a plasmid or chromosome. In addition, multiple copies of any regulatory region, promoter, gene, and/or gene cassette may be present in the bacterium, wherein one or more copies of the regulatory region, promoter, gene, and/or gene cassette may be mutated or otherwise altered as described herein. In some embodiments, the genetically engineered bacteria are engineered to comprise multiple copies of the same regulatory region, promoter, gene, and/or gene cassette in order to enhance copy number or to comprise multiple different components of a gene cassette performing multiple different functions. In some embodiments, the genetically engineered bacteria comprise a gene encoding a phenylalanine-metabolizing enzyme that is operably linked to a directly or indirectly inducible promoter that is not associated with said gene in nature, e.g., an FNR promoter operably linked to a gene encoding PAL or a ParaBAD promoter operably linked to LAAD.

“Constitutive promoter” refers to a promoter that is capable of facilitating continuous transcription of a coding sequence or gene under its control and/or to which it is operably linked. Constitutive promoters, inducible promoters, and variants thereof are well known in the art and described in PCT/US2016/032562 and PCT/US2016/062369, the contents of which are hereby incorporated by reference. Constitutive promoters and variants are well known in the art and include, but are not limited to, BBa_J23100, a constitutive Escherichia coli σ^{S} promoter (e.g., an osmY promoter (International Genetically Engineered Machine (iGEM) Registry of Standard Biological Parts Name BBa_J45992; BBa_J45993)), a constitutive Escherichia coli σ³² promoter (e.g., htpG heat shock promoter (BBa_J45504)), a constitutive Escherichia coli σ⁷⁰ promoter (e.g., lacq promoter (BBa_J54200; BBa_J56015), E. coli CreABCD phosphate sensing operon promoter (BBa_J64951), GlnRS promoter (BBa_K088007), lacZ promoter (BBa_K119000; BBa_K119001); M13K07 gene I promoter (BBa_M13101); M13K07 gene II promoter (BBa_M13102), M13K07 gene III promoter (BBa_M13103), M13K07 gene IV promoter (BBa_M13104), M13K07 gene V promoter (BBa_M13105), M13K07 gene VI promoter (BBa_M13106), M13K07 gene VIII promoter (BBa_M13108), M13110 (BBa_M13110)), a constitutive Bacillus subtilis aApromoter (e.g., promoter veg (BBa_K143013), promoter 43 (BBa_K143013), P_{liaG} (BBa_K823000), P_{lepA} (BBa_K823002), P_{veg} (BBa_K823003)), a constitutive Bacillus subtilis σ^{B} promoter (e.g., promoter ctc (BBa_K143010), promoter gsiB (BBa_K143011)), a Salmonella promoter (e.g., Pspv2 from Salmonella (BBa_K112706), Pspv from Salmonella (BBa_K112707)), a bacteriophage T7 promoter (e.g., T7 promoter (BBa_I712074; BBa_I719005; BBa_J34814; BBa_J64997; BBa_K113010; BBa_K113011; BBa_K113012; BBa_R0085; BBa_R0180; BBa_R0181; BBa_R0182; BBa_R0183; BBa_Z0251; BBa_Z0252; BBa_Z0253)), a bacteriophage SP6 promoter (e.g., SP6 promoter (BBa_J64998)), and functional fragments thereof.

“Gut” refers to the organs, glands, tracts, and systems that are responsible for the transfer and digestion of food, absorption of nutrients, and excretion of waste. In humans, the gut comprises the gastrointestinal (GI) tract, which starts at the mouth and ends at the anus, and additionally comprises the esophagus, stomach, small intestine, and large intestine. The gut also comprises accessory organs and glands, such as the spleen, liver, gallbladder, and pancreas. The upper gastrointestinal tract comprises the esophagus, stomach, and duodenum of the small intestine. The lower gastrointestinal tract comprises the remainder of the small intestine, i.e., the jejunum and ileum, and all of the large intestine, i.e., the cecum, colon, rectum, and anal canal. Bacteria can be found throughout the gut, e.g., in the gastrointestinal tract, and particularly in the intestines. In some embodiments, the genetically engineered microorganisms are active (e.g., express one or more PMEs) in the stomach and/or the gut, i.e., small and/or large intestine. Without wishing to be bound by theory, the engineered microorganisms described herein may be particularly effective in the small intestine, because amino acid absorption, e.g., phenylalanine absorption, occurs in the small intestine. Through the prevention or reduction of phenylalanine uptake into the blood, increased levels and resulting Phe toxicity can be avoided. Additionally, extensive enterorecirculation of amino acids between the intestine and the body may allow the removal of systemic phenylalanine in PKU (e.g., described by Chang et al., in a rat model of PKU (Chang et al., A new theory of enterorecirculation of amino acids and its use for depleting unwanted amino acids using oral enzyme-artificial cells, as in removing phenylalanine in phenylketonuria; Artif Cells Blood Substit Immobil Biotechnol. 1995; 23(1):1-21)). Phenylalanine from the blood circulates into the small intestine and can be cleared by microorganisms which are active at this location. In some embodiments, the genetically engineered microorganisms transit through the small intestine. In some embodiments, the genetically engineered microorganisms have increased residence time in the small intestine. In some embodiments, the genetically engineered microorganisms colonize the small intestine. In some embodiments, the genetically engineered microorganisms do not colonize the small intestine. In some embodiments, the genetically engineered microorganisms have increased residence time in the gut. In some embodiments, the genetically engineered microorganisms colonize the gut. In some embodiments, the genetically engineered microorganisms do not colonize the gut.

“Microorganism” refers to an organism or microbe of microscopic, submicroscopic, or ultramicroscopic size that typically consists of a single cell. Examples of microorganisms include bacteria, viruses, parasites, fungi, certain algae, and protozoa. In some aspects, the microorganism is engineered (“engineered microorganism”) to produce one or more therapeutic molecules or proteins of interest. In certain aspects, the microorganism is engineered to take up and catabolize certain metabolites or other compounds from its environment, e.g., the gut. In certain aspects, the microorganism is engineered to synthesize certain beneficial metabolites or other compounds (synthetic or naturally occurring) and release them into its environment. In certain embodiments, the engineered microorganism is an engineered bacterium. In certain embodiments, the engineered microorganism is an engineered virus.

“Non-pathogenic bacteria” refer to bacteria that are not capable of causing disease or harmful responses in a host. In some embodiments, non-pathogenic bacteria are Gram-negative bacteria. In some embodiments, non-pathogenic bacteria are Gram-positive bacteria. In some embodiments, non-pathogenic bacteria are commensal bacteria, which are present in the indigenous microbiota of the gut. Examples of non-pathogenic bacteria include, but are not limited to, Bacillus, Bacteroides, Bifidobacterium, Brevibacteria, Clostridium, Enterococcus, Escherichia, Lactobacillus, Lactococcus, Saccharomyces, and Staphylococcus, e.g., Bacillus coagulans, Bacillus subtilis, Bacteroides fragilis, Bacteroides subtilis, Bacteroides thetaiotaomicron, Bifidobacterium bifidum, Bifidobacterium infantis, Bifidobacterium lactis, Bifidobacterium longum, Clostridium butyricum, Enterococcus faecium, Escherichia coli, Lactobacillus acidophilus, Lactobacillus bulgaricus, Lactobacillus casei, Lactobacillus johnsonii, Lactobacillus paracasei, Lactobacillus plantarum, Lactobacillus reuteri, Lactobacillus rhamnosus, Lactococcus lactis, and Saccharomyces boulardii (Sonnenborn et al., 2009; Dinleyici et al., 2014; U.S. Pat. Nos. 6,835,376; 6,203,797; 5,589,168; 7,731,976). Naturally pathogenic bacteria may be genetically engineered to provide reduce or eliminate pathogenicity.

“Probiotic” is used to refer to live, non-pathogenic microorganisms, e.g., bacteria, which can confer health benefits to a host organism that contains an appropriate amount of the microorganism. In some embodiments, the host organism is a mammal. In some embodiments, the host organism is a human. Some species, strains, and/or subtypes of non-pathogenic bacteria are currently recognized as probiotic. Examples of probiotic bacteria include, but are not limited to, Bifidobacteria, Escherichia, Lactobacillus, and Saccharomyces, e.g., Bifidobacterium bifidum, Enterococcus faecium, Escherichia coli, Escherichia coli strain Nissle, Lactobacillus acidophilus, Lactobacillus bulgaricus, Lactobacillus paracasei, Lactobacillus plantarum, and Saccharomyces boulardii (Dinleyici et al., 2014; U.S. Pat. Nos. 5,589,168; 6,203,797; 6,835,376). The probiotic may be a variant or a mutant strain of bacterium (Arthur et al., 2012; Cuevas-Ramos et al., 2010; Olier et al., 2012; Nougayrede et al., 2006). Non-pathogenic bacteria may be genetically engineered to enhance or improve desired biological properties, e.g., survivability. Non-pathogenic bacteria may be genetically engineered to provide probiotic properties. Probiotic bacteria may be genetically engineered to enhance or improve probiotic properties.

As used herein, “stable” microorganism is used to refer to a microorganism host cell carrying non-native genetic material, e.g., a PAL gene, which is incorporated into the host genome or propagated on a self-replicating extra-chromosomal plasmid, such that the non-native genetic material is retained, expressed, and/or propagated, e.g., under particular conditions. The stable microorganism is capable of survival and/or growth in vitro, e.g., in medium, and/or in vivo, e.g., in the gut. For example, the stable microorganisms may be a genetically modified bacterium comprising a PAL gene, e.g., mutant PAL, in which the plasmid or chromosome carrying the PAL gene is stably maintained in the host cell, such that PAL can be expressed in the host cell, and the host cell is capable of survival and/or growth in vitro and/or in vivo. In some embodiments, copy number affects the stability of expression of the non-native genetic material, e.g., a PAL gene or a PAH gene. In some embodiments, copy number affects the level of expression of the non-native genetic material, e.g., a PAL gene or a PAH gene.

As used herein, the terms “modulate” and “treat” and their cognates refer to an amelioration of a disease, disorder, and/or condition, or at least one discernible symptom thereof. In another embodiment, “modulate” and “treat” refer to an amelioration of at least one measurable physical parameter, not necessarily discernible by the patient. In another embodiment, “modulate” and “treat” refer to inhibiting the progression of a disease, disorder, and/or condition, either physically (e.g., stabilization of a discernible symptom), physiologically (e.g., stabilization of a physical parameter), or both. In another embodiment, “modulate” and “treat” refer to slowing the progression or reversing the progression of a disease, disorder, and/or condition. As used herein, “prevent” and its cognates refer to delaying the onset or reducing the risk of acquiring a given disease, disorder and/or condition or a symptom associated with such disease, disorder, and/or condition. Those in need of treatment may include individuals already having a particular medical disease, as well as those at risk of having, or who may ultimately acquire the disease. The need for treatment is assessed, for example, by the presence of one or more risk factors associated with the development of a disease, the presence or progression of a disease, or likely receptiveness to treatment of a subject having the disease. Primary hyperphenylalaninemia, e.g., PKU, is caused by inborn genetic mutations for which there are no known cures. Hyperphenylalaninemia can also be secondary to other conditions, e.g., liver diseases. Treating hyperphenylalaninemia may encompass reducing or eliminating excess phenylalanine and/or associated symptoms and does not necessarily encompass the elimination of the underlying disease.

In some embodiments, the discernible symptom is measured in a subject at baseline, e.g., prior to administration of the genetically engineered bacterium, and measured in the subject after a suitable period of time after administration of the genetically engineered bacterium. In some embodiments, the baseline measurement is made in a fasted state, e.g., prior to a meal, e.g., in a subject having phenylketonuria.

In some embodiments, the discernible symptom to be assessed is phenylalanine, e.g., excess levels in the blood, e.g., at least 2 mg/dL, at least 4 mg/dL, at least 6 mg/dL, at least 8 mg/dL, at least 10 mg/dL, at least 12 mg/dL, at least 14 mg/dL, at least 16 mg/dL, at least 18 mg/dL, at least 20 mg/dL, or at least 25 mg/dL or more. In some embodiments, the discernible symptom to be assessed is phenylalanine, e.g., excess levels in the blood, e.g., at least 360 μmol/L, at least 600 μmol/L, at least 1200 μmol/L, or more, or at least 360 μmol/L to 600 μmol/L, at least 600 to 1200 μmol/L or at least greater than 1200 μmol/L.

In some embodiments, the subject to be treated has a baseline level of phenylalanine of at least 600 μmol/L. In some embodiments, the subject to be treated has a baseline level of phenylalanine of at least 360 μmol/L.

In some embodiments, the methods herein reduce phenylalanine levels, e.g., in the blood, after administration of the genetically engineered bacteria as compared to baseline levels in the subject before administration. In some embodiments, the methods herein reduce phenylalanine levels, e.g., in the blood, by at least 5%, at least 10%, at least 15%, at least 20%, at least 25%, at least 30%, at least 35%, at least 40%, at least 45%, at least 50%, at least 55%, at least 60%, at least 70%, at least 80%, at least 90% or at least 95% or more as compared to baseline levels in the subject before administration.

In some embodiments, the discernible symptom to be assessed is cognitive function using the Cambridge Neuropsychological Test Automated Battery (CANTAB), i.e., Changes in CANTAB item scores from baseline prior to treatment.

As used herein a “pharmaceutical composition” refers to a preparation of genetically engineered bacteria disclosed herein with other components such as a physiologically suitable carrier and/or excipient.

The phrases “physiologically acceptable carrier” and “pharmaceutically acceptable carrier” which may be used interchangeably refer to a carrier or a diluent that does not cause significant irritation to an organism and does not abrogate the biological activity and properties of the administered bacterial compound. An adjuvant is included under these phrases.

The term “excipient” refers to a substance added to a pharmaceutical composition to further facilitate administration of an active ingredient. Examples include, but are not limited to, calcium bicarbonate, calcium phosphate, various sugars and types of starch, cellulose derivatives, gelatin, vegetable oils, polyethylene glycols, and surfactants, including, for example, polysorbate 20.

The terms “therapeutically effective dose” and “therapeutically effective amount” are used to refer to an amount of a compound that results in prevention, delay of onset of symptoms, or amelioration of symptoms of a condition, e.g., hyperphenylalaninemia. A therapeutically effective amount may, for example, be sufficient to treat, prevent, reduce the severity, delay the onset, and/or reduce the risk of occurrence of one or more symptoms of a disease or condition associated with excess phenylalanine levels. A therapeutically effective amount, as well as a therapeutically effective frequency of administration, can be determined by methods known in the art and discussed below.

As used herein, the term “polypeptide” includes “polypeptide” as well as “polypeptides,” and refers to a molecule composed of amino acid monomers linearly linked by amide bonds (i.e., peptide bonds). The term “polypeptide” refers to any chain or chains of two or more amino acids, and does not refer to a specific length of the product. Thus, “peptides,” “dipeptides,” “tripeptides, “oligopeptides,” “protein,” “amino acid chain,” or any other term used to refer to a chain or chains of two or more amino acids, are included within the definition of “polypeptide,” and the term “polypeptide” may be used instead of, or interchangeably with any of these terms. The term “dipeptide” refers to a peptide of two linked amino acids. The term “tripeptide” refers to a peptide of three linked amino acids. The term “polypeptide” is also intended to refer to the products of post-expression modifications of the polypeptide, including but not limited to glycosylation, acetylation, phosphorylation, amidation, derivatization, proteolytic cleavage, or modification by non-naturally occurring amino acids. A polypeptide may be derived from a natural biological source or produced by recombinant technology. In other embodiments, the polypeptide is produced by the genetically engineered bacteria or virus of the current invention. A polypeptide may be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides may have a defined three-dimensional structure, although they do not necessarily have such structure. Polypeptides with a defined three-dimensional structure are referred to as folded, and polypeptides, which do not possess a defined three-dimensional structure, but rather can adopt a large number of different conformations, are referred to as unfolded. The term “peptide” or “polypeptide” may refer to an amino acid sequence that corresponds to a protein or a portion of a protein or may refer to an amino acid sequence that corresponds with non-protein sequence, e.g., a sequence selected from a regulatory peptide sequence, leader peptide sequence, signal peptide sequence, linker peptide sequence, and other peptide sequence.

The terms “phage” and “bacteriophage” are used interchangeably herein. Both terms refer to a virus that infects and replicates within a bacterium. As used herein “phage” or bacteriophage” collectively refers to prophage, lysogenic, dormant, temperate, intact, defective, cryptic, and satellite phage, phage tail bacteriocins, tailiocins, and gene transfer agents. As used therein the term “prophage” refers to the genomic material of a bacteriophage, which is integrated into a replicon of the host cell and replicates along with the host. The prophage may be able to produce phages if specifically activated. In some cases, the prophage is not able to produce phages or has never done so (i.e., defective or cryptic prophages). In some cases, prophage also refers to satellite phages. The terms “prophage” and “endogenous phage” are used interchangeably herein. “Endogenous phage” or “endogenous prophage” also refers to a phage that is present in the natural state of a bacterium (and its parental strain). As used herein the term “phage knockout” or “inactivated phage” refers to a phage which has been modified so that it can either no longer produce and/or package phage particles or it produces fewer phage particles than the wild-type phage sequence. In some embodiments, the inactivated phage or phage knockout refers to the inactivation of a temperate phage in its lysogenic state, i.e., to a prophage. Such a modification refers to a mutation in the phage; such mutations include insertions, deletions (partial or complete deletion of phage genome), substitutions, inversions, at one or more positions within the phage genome, e.g., within one or more genes within the phage genome. As used herein the adjectives “phage-free”, “phage free” and “phageless” are used interchangeably to characterize a bacterium or strain which contains one or more prophages, one or more of which have been modified. The modification can result in a loss of the ability of the prophage to be induced or release phage particles. Alternatively, the modification can result in less efficient or less frequent induction, or less efficient or less frequent phage release as compared to the isogenic strain without the modification. Ability to induce and release phage can be measured using a plaque assay as described herein.

As used herein phage induction refers to the part of the life cycle of a lysogenic prophage, in which the lytic phage genes are activated, phage particles are produced, and lysis occurs.

An “isolated” polypeptide or a fragment, variant, or derivative thereof refers to a polypeptide that is not in its natural milieu. No particular level of purification is required. Recombinantly produced polypeptides and proteins expressed in host cells, including but not limited to bacterial or mammalian cells, are considered isolated for purposed of the invention, as are native or recombinant polypeptides which have been separated, fractionated, or partially or substantially purified by any suitable technique. Recombinant peptides, polypeptides or proteins refer to peptides, polypeptides or proteins produced by recombinant DNA techniques, i.e. produced from cells, microbial or mammalian, transformed by an exogenous recombinant DNA expression construct encoding the polypeptide. Proteins or peptides expressed in most bacterial cultures will typically be free of glycan. Fragments, derivatives, analogs or variants of the foregoing polypeptides, and any combination thereof are also included as polypeptides. The terms “fragment,” “variant,” “derivative” and “analog” include polypeptides having an amino acid sequence sufficiently similar to the amino acid sequence of the original peptide and include any polypeptides, which retain at least one or more properties of the corresponding original polypeptide. Fragments of polypeptides of the present invention include proteolytic fragments, as well as deletion fragments. Fragments also include specific antibody or bioactive fragments, or immunologically active fragments derived from any polypeptides described herein. Variants may occur naturally or be non-naturally occurring. Non-naturally occurring variants may be produced using mutagenesis methods known in the art. Variant polypeptides may comprise conservative or non-conservative amino acid substitutions, deletions or additions.

Polypeptides also include fusion proteins. As used herein, the term “variant” includes a fusion protein, which comprises a sequence of the original peptide or sufficiently similar to the original peptide. “Derivatives” include but are not limited to peptides, which contain one or more naturally occurring amino acid derivatives of the twenty standard amino acids. “Similarity” between two peptides is determined by comparing the amino acid sequence of one peptide to the sequence of a second peptide. An amino acid of one peptide is similar to the corresponding amino acid of a second peptide if it is identical or a conservative amino acid substitution. Conservative substitutions include those described in Dayhoff, M. O., ed., The Atlas of Protein Sequence and Structure 5, National Biomedical Research Foundation, Washington, D.C. (1978), and in Argos, EMBO J. 8 (1989), 779-785. For example, amino acids belonging to one of the following groups represent conservative changes or substitutions: -Ala, Pro, Gly, Gln, Asn, Ser, Thr; -Cys, Ser, Tyr, Thr; -Val, Ile, Leu, Met, Ala, Phe; -Lys, Arg, His; -Phe, Tyr, Trp, His; and -Asp, Glu.

As used herein, the term “sufficiently similar” means a first amino acid sequence that contains a sufficient or minimum number of identical or equivalent amino acid residues relative to a second amino acid sequence such that the first and second amino acid sequences have a common structural domain and/or common functional activity. For example, amino acid sequences that comprise a common structural domain that is at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100%, identical are defined herein as sufficiently similar. Preferably, variants will be sufficiently similar to the amino acid sequence of the peptides of the invention. Such variants generally retain the functional activity of the peptides of the present invention. Variants include peptides that differ in amino acid sequence from the native and wt peptide, respectively, by way of one or more amino acid deletion(s), addition(s), and/or substitution(s). These may be naturally occurring variants as well as artificially designed ones.

As used herein the term “codon-optimized sequence” refers to a sequence, which was modified from an existing coding sequence, or designed, for example, to improve translation in an expression host cell or organism of a transcript RNA molecule transcribed from the coding sequence, or to improve transcription of a coding sequence. Codon optimization includes, but is not limited to, processes including selecting codons for the coding sequence to suit the codon preference of the expression host organism.

Many organisms display a bias or preference for use of particular codons to code for insertion of a particular amino acid in a growing polypeptide chain. Codon preference or codon bias, differences in codon usage between organisms, is allowed by the degeneracy of the genetic code, and is well documented among many organisms. Codon bias often correlates with the efficiency of translation of messenger RNA (mRNA), which is in turn believed to be dependent on, inter alia, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

As used herein, the term “transporter” is meant to refer to a mechanism. e.g., protein or proteins, for importing a molecule, e.g., amino acid, toxin, metabolite, substrate, etc. into the microorganism from the extracellular milieu.

The articles “a” and “an,” as used herein, should be understood to mean “at least one,” unless clearly indicated to the contrary.

The phrase “and/or,” when used between elements in a list, is intended to mean either (1) that only a single listed element is present, or (2) that more than one element of the list is present. For example, “A, B, and/or C” indicates that the selection may be A alone; B alone; C alone; A and B; A and C; B and C; or A, B, and C. The phrase “and/or” may be used interchangeably with “at least one of” or “one or more of” the elements in a list.

### Methods for Reducing Hyperphenylalaninemia

This disclosure provides methods of reducing hyperphenylalaninemia and/or treating a disease associated with hyperphenylalaninemia, e.g., PKU, or symptom(s) associated with hyperphenylalaninemia.

In some embodiments, the disease is selected from the group consisting of: phenylketonuria, classical or typical phenylketonuria, atypical phenylketonuria, mild hyperphenylalaninemia, nonphenylketonuric hyperphenylalaninemia, phenylalanine hydroxylase deficiency, cofactor deficiency, dihydropteridine reductase deficiency, 6-pyruvoyl tetrahydropterin synthase deficiency, DNAJC12 deficiency, and Segawa's disease.

In some embodiments, hyperphenylalaninemia is secondary to other conditions, e.g., liver diseases. In some embodiments, the invention provides methods for reducing, ameliorating, or eliminating one or more symptom(s) associated with these diseases, including but not limited to neurological deficits, cognitive impairment, encephalopathy, epilepsy, eczema, reduced growth, microcephaly, tremor, limb spasticity, and/or hypopigmentation. In some embodiments, the subject to be treated is a human patient.

In some embodiments, the method of treatment and/or method of reducing hyperphenylalaninemia comprises administering a genetically engineered bacterium comprising one or more gene(s) encoding PAL. Exemplary PAL sequences are disclosed herein, e.g., at Table 6. Amino acid sequences having at least 80%, at least 85%, at least 90%, at least 95%, at least 96%, at least 97%, at least 98%, or at least 99% identity to the amino acid sequences disclosed herein or functional fragments thereof are contemplated. Exemplary nucleotide sequences encoding these amino acid sequences are provided herein (see, e.g., SEQ ID NO: 508), and other suitable nucleotide sequences encoding these amino acid sequences would be appreciated by one of skill in the art. Nucleotide sequences having at least 80%, at least 85%, at least 90%, at least 95%, at least 96%, at least 97%, at least 98%, or at least 99% identity to such nucleotide sequences, including codon-optimized nucleotide sequences thereof, are contemplated.

TABLE 6
Exemplary PAL Sequences

| Name | Amino Acid Sequence |
|---|---|
| Photorhabdus | MKAKDVQPTIIINKNGLISLEDIYDIAIKQKKVEISTEITELLTHGREKLE |
| luminescens | EKLNSGEVIYGINTGFGGNANLVVPFEKIAEHQQNLLTFLSAGTGDYM |
| PAL3 Wild-type | SKPCIKASQFTMLLSVCKGWSATRPIVAQAIVDHINHDIVPLVPRYGSV |
| (SEQ ID NO: | GASGDLIPLSYIARALCGIGKVYYMGAEIDAAEAIKRAGLTPLSLKAKE |
| 500) | GLALINGTRVMSGISAITVIKLEKLFKASISAIALAVEALLASHEHYDAR |
| | IQQVKNHPGQNAVASALRNLLAGSTQVNLLSGVKEQANKACRHQEIT |
| | QLNDTLQEVYSIRCAPQVLGIVPESLATARKILEREVISANDNPLIDPEN |
| | GDVLHGGNFMGQYVARTMDALKLDIALIANHLHAIVALMMDNRFSR |
| | GLPNSLSPTPGMYQGFKGVQLSQTALVAAIRHDCAASGIHTLATEQYN |
| | QDIVSLGLHAAQDVLEMEQKLRNIVSMTILVVCQAIHLRGNISEIAPET |
| | AKFYHAVREISSPLITDRALDEDIIRIADAIINDQLPLPEIMLEE |
| mPAL1 | MKAKDVQPTIIINKNGLISLEDIYDIAIKQKKVEISTEITELLTHGREKLE |
| (SEQ ID NO: | EKLNSGEVIYGINTGFGGNANLVVPFEKIAEHQQNLLTFLGAGTGDYM |
| 501) | SKPCIKASQFTMLLSVCKGWSATRPIVAQAIVDMINHDIVPLVPRYGSV |
| | GASGDLIPLSYIARALCGKGKVYYMGAEIDAAEAIKRAGLTPLSLKAK |
| | EGLALINGTRVMSGISAITVIKLEKLFKASISAIALAVEALLASHEHYDA |
| | RIQQVKNHPGQNAVASALRNLLAGSTQVNLLSGVKEQANKACRHQEI |
| | TQLNDTLQEVYSIRCAPQVLGIVPESLATARKILEREVISANDNPLIDPE |
| | NGDVLHGGNFMGQYVARTMDALKLDIALIANHLHAIVALMMDNRFS |
| | RGLPNSLSPTPGMYQGFKGVQLSQTALVAAIRHDCAASGIHTIATEQY |
| | NQDIVSLGLHAAQDVLEMEQKLRNIVSMTILVACQAIHLRGNISEIAPE |
| | TAKFYHAVREISSPLITDRALDEDIIRIADAIINDQLPLPEIMLEE |
| mPAL2 | MKAKDVQPTIIINKNGLISLEDIYDIAIKQKKVEISTEITELLTHGREKLE |
| (SEQ ID NO: | EKLNSGEVIYGINTGFGGNANLVVPFEKIAEHQQNLLTFLGAGTGDYM |
| 502) | SKPCIKASQFTMLLSVCKGWSATRPIVAQAIVDFINHDIVPLVPRYGSV |
| | GASGDLIPLSYIARALCGIGKVYYMGAEIDAAEAIKRAGLTPLSLKAKE |
| | GLALINGTRVMSGISAITVIKLEKLFKASISAIALAVEALLASHEHYDAR |
| | IQQVKNHPGQNAVASALRNLLAGSTQVNLLSGVKEQANKACRHQEIT |
| | QLNDTLQEVYSIRCAPQVLGIVPESLATARKILEREVISANDNPLIDPEN |
| | GDVLHGGNFMGQYVARTMDALKLDIALIANHLHAIVALMMDNRFSR |
| | GLPNSLSPTPGMYQGFKGVQLSQTALVAAIRHDCAASGIHTLSTEQYN |
| | QDIVSLGLHAAQDVLEMEQKLRNIVSMTILVACQAIHLRGNISEIAPET |
| | AKFYHAVREISSPLITDRALDEDIIRIADAIINDQLPLPEIMLEE |
| mPAL3 | MKAKDVQPTIIINKNGLISLEDIYDIAIKQKKVEISTEITELLTHGREKLE |
| (SEQ ID NO: | EKLNSGEVIYGINTGFGGNANLVVPFEKIAEHQQNLLTFLGAGTGDYM |
| 503) | SKPCIKASQFTMLLSVCKGWSATRPIVAQAIVDFINHDIVPLVPRYGSV |
| | GASGDLIPLSYIARALCGIGKVYYMGAEIDAAEAIKRAGLTPLSLKAKE |
| | GLALINGTRVMSGISAITVIKLEKLFKASISAIALAVEALLASHEHYDAR |
| | IQQVKNHPGQNAVASTLRNLLAGSTQVNLLSGVKEQANKACRHQEIT |
| | QLNDTLQEVYSIRCAPQVLGIVPESLATARKILEREVISANDNPLIDPEN |
| | GDVLHGGNFMGQYVARTMDALKLDIALIANHLHAIVALMMDNRFSR |
| | GLPNSLSPTPGMYQGFKGVQLSQTALVAAIRHDCAASGIHTLATEQYN |
| | QDIVSLGLHAAQDVLEMEQKLRNIVSMTILVACQAIHLRGNISEIAPET |
| | AKFYHAVREISSPLITDRALDEDIIRIADAIINDQLPLPEIMLEE |

In some embodiments, the bacterium disclosed herein comprises a nucleotide sequence that encodes a PAL sequence that is at least 80%, at least 85%, at least 90%, at least 91%, at least 92%, at least 93%, at least 94%, at least 95%, at least 96%, at least 97%, at least 98%, at least 99%, or 100% identical to a PAL amino acid sequence in Table 6 or a functional fragment thereof. In some embodiments, the method of treatment, e.g., for PKU, and/or method of reducing hyperphenylalaninemia comprises administering a genetically engineered bacterium comprising a PAL derived from wild-type Photorhabdus luminescens PAL, e.g., a PAL gene derived from Anabaena variabilis (“PAL1” herein) or a PAL gene derived from Photorhabdus luminescens (“PAL3” herein).

In some embodiments, the method of treatment, e.g., for PKU, and/or method of reducing hyperphenylalaninemia comprises administering a genetically engineered bacterium comprising a mutant PAL derived from wild-type Photorhabdus luminescens PAL, e.g., SEQ ID NO: 500. In some embodiments, the method of treatment and/or method of reducing hyperphenylalaninemia comprises administering a genetically engineered bacterium comprising a mutant PAL with mutations in one or more amino acid positions selected from 92, 133, 167, 432, 470, 433, 263, 366 and 396 compared to positions in wild-type PAL, e.g., Photorhabdus luminescens PAL, e.g., SEQ ID NO: 500. In some embodiments, the method of treatment and/or method of reducing hyperphenylalaninemia comprises administering a genetically engineered bacterium comprising a mutant PAL with mutations in one or more amino acid positions selected from S92, H133, I167, L432, V470, A433, A263, K366, and/or L396 compared to positions in wild-type PAL, e.g., Photorhabdus luminescens PAL, e.g., SEQ ID NO: 500. In some embodiments, the method of treatment and/or method of reducing hyperphenylalaninemia comprises administering a genetically engineered bacterium comprising a mutant PAL with mutations in one or more amino acid positions selected from S92G, H133M, H133F, I167K, L432I, V470A, A433S, A263T, K366K (e.g., silent mutation in polynucleotide sequence), and/or L396L (e.g., silent mutation in polynucleotide sequence) compared to positions in wild-type PAL, e.g., Photorhabdus luminescens PAL, e.g., SEQ ID NO: 500.

In some embodiments, the method of treatment, e.g., for PKU, and/or method of reducing hyperphenylalaninemia comprises administering a genetically engineered bacterium comprising a mutant PAL with mutations in one or more amino acid positions selected from S92G, H133M, I167K, L432I, and V470A compared to positions in wild-type PAL, e.g., Photorhabdus luminescens PAL, e.g., SEQ ID NO: 500. In some embodiments, the method of treatment and/or method of reducing hyperphenylalaninemia comprises administering a genetically engineered bacterium comprising a mutant PAL with mutations in one or more amino acid positions selected from S92G, H133F, A433S, and V470A compared to positions in wild-type PAL, e.g., Photorhabdus luminescens PAL, e.g., SEQ ID NO: 500. In some embodiments, the method of treatment and/or method of reducing hyperphenylalaninemia comprises administering a genetically engineered bacterium comprising a mutant PAL with mutations in one or more amino acid positions selected from S92G. H133F, A263T, K366K (e.g., silent mutation in polynucleotide sequence), L396L (e.g., silent mutation in polynucleotide sequence), and V470A compared to positions in wild-type PAL, e.g., Photorhabdus luminescens PAL, e.g., SEQ ID NO: 500.

In one embodiment, the phenylalanine ammonia lyase (PAL) gene encodes a polypeptide has at least about 80% identity with SEQ ID NO: 500. Accordingly, in one embodiment, the PAL gene encodes a polypeptide has at least about 90% identity with SEQ ID NO: 500. Accordingly, in one embodiment, the PAL gene encodes a polypeptide has at least about 95% identity with SEQ ID NO: 500. Accordingly, in one embodiment, the PAL gene encodes a polypeptide has at least about 85%, 86%, 87%, 88%, 89%, 90%, 91%, 92%, 93%, 94%, 95%, 96%, 97%, 98%, or 99% identity with SEQ ID NO: 500. In another embodiment, the PAL gene encodes a polypeptide comprises SEQ ID NO: 500. In yet another embodiment, the PAL gene encodes a polypeptide consists of SEQ ID NO: 500.

In one embodiment, the phenylalanine ammonia lyase (PAL) gene encodes a polypeptide has at least about 80% identity with SEQ ID NO: 503. Accordingly, in one embodiment, the PAL gene encodes a polypeptide has at least about 90% identity with SEQ ID NO: 503. Accordingly, in one embodiment, the PAL gene encodes a polypeptide has at least about 95% identity with SEQ ID NO: 503. Accordingly, in one embodiment, the PAL gene encodes a polypeptide has at least about 85%, 86%, 87%, 88%, 89%, 90%, 91%, 92%, 93%, 94%, 95%, 96%, 97%, 98%, or 99% identity with SEQ ID NO: 503. In another embodiment, the PAL gene encodes a polypeptide comprises SEQ ID NO: 503. In yet another embodiment, the PAL gene encodes a polypeptide consists of SEQ ID NO: 503.

In some embodiments, the method of treatment, e.g., for PKU, and/or method of reducing hyperphenylalaninemia comprises administering a genetically engineered bacterium comprising PAL1. In some embodiments, the method of treatment and/or method of reducing hyperphenylalaninemia comprises administering a genetically engineered bacterium comprising PAL3. In some embodiments, the method of treatment and/or method of reducing hyperphenylalaninemia comprises administering a genetically engineered bacterium comprising mPAL1. In some embodiments, the method of treatment and/or method of reducing hyperphenylalaninemia comprises administering a genetically engineered bacterium comprising mPAL2. In some embodiments, the method of treatment and/or method of reducing hyperphenylalaninemia comprises administering a genetically engineered bacterium comprising mPAL3.

In some embodiments, the method of treatment, e.g., for PKU, and/or method of reducing hyperphenylalaninemia comprises administering a genetically engineered bacterium comprising PAL and further comprising additional PME(s), e.g., PAH, LAAD, and/or phenylalanine transporter(s). Exemplary PMEs and combinations thereof are known the in art, see, e.g., PCT/US2016/032562 and PCT/US2016/062369, the contents of which are hereby incorporated by reference.

In some embodiments, the method of treatment, e.g., for PKU, and/or method of reducing hyperphenylalaninemia comprises administering a genetically engineered bacterium comprising PAL and further comprising one or more genes encoding a phenylalanine transporter, e.g., pheP.

In one embodiment, the phenylalanine transporter gene encodes a polypeptide has at least about 80% identity with SEQ ID NO: 509. Accordingly, in one embodiment, the phenylalanine transporter gene encodes a polypeptide has at least about 90% identity with SEQ ID NO: 509. Accordingly, in one embodiment, the phenylalanine transporter gene encodes a polypeptide has at least about 95% identity with SEQ ID NO: 509. Accordingly, in one embodiment, the phenylalanine transporter gene encodes a polypeptide has at least about 85%, 86%, 87%, 88%, 89%, 90%, 91%, 92%, 93%, 94%, 95%, 96%, 97%, 98%, or 99% identity with SEQ ID NO: 509. In another embodiment, the phenylalanine transporter gene encodes a polypeptide comprises SEQ ID NO: 509. In yet another embodiment, the phenylalanine transporter gene encodes a polypeptide consists of SEQ ID NO: 509.

In some embodiments, the method of treatment, e.g., for PKU, and/or method of reducing hyperphenylalaninemia comprises administering a genetically engineered bacterium comprising PAL and further comprising one or more genes encoding LAAD.

In one embodiment, the L-amino acid deaminase (LAAD) gene encodes a polypeptide has at least about 80% identity with SEQ ID NO: 510. Accordingly, in one embodiment, the LAAD gene encodes a polypeptide has at least about 90% identity with SEQ ID NO: 510. Accordingly, in one embodiment, the LAAD gene encodes a polypeptide has at least about 95% identity with SEQ ID NO: 510. Accordingly, in one embodiment, the LAAD gene encodes a polypeptide has at least about 85%, 86%, 87%, 88%, 89%, 90%, 91%, 92%, 93%, 94%, 95%, 96%, 97%, 98%, or 99% identity with SEQ ID NO: 510. In another embodiment, the LAAD gene encodes a polypeptide comprises SEQ ID NO: 510. In yet another embodiment, the LAAD gene encodes a polypeptide consists of SEQ ID NO: 510.

In some embodiments, the method of treatment, e.g., for PKU, and/or method of reducing hyperphenylalaninemia comprises administering a genetically engineered bacterium comprising PAL and further comprising a transcriptional regulator, e.g., a non-native transcriptional regulator as described herein. In these embodiments, the PME, e.g., PAL, mutant PAL, phenylalanine transporter, and/or transcriptional regulator may be operably linked to one or more promoters as disclosed herein, e.g., a constitutive promoter, an inducible promoter, a thermoregulated promoter, an oxygen-level dependent promoter, etc.

In some embodiments, the method of treatment, e.g., for PKU, and/or method of reducing hyperphenylalaninemia comprises administering a genetically engineered bacterium comprising PAL and further comprising one or more gene sequences relating to biosafety and/or biocontainment as described herein, e.g., a kill-switch, gene guard system, essential gene for cell growth and/or survival, thyA, dapA, auxotrophy, etc.

In some embodiments, the method of treatment, e.g., for PKU, and/or method of reducing hyperphenylalaninemia comprises administering a genetically engineered bacterium comprising two copies of a gene encoding PAL, e.g., PAL1, PAL3, mutant PAL, e.g., mPAL1, mPAL2, or mPAL3.

In some embodiments, the method of treatment, e.g., for PKU, and/or method of reducing hyperphenylalaninemia comprises administering a genetically engineered bacterium comprising three copies of a gene encoding PAL, e.g., PAL1, PAL3, mutant PAL, e.g., mPAL1, mPAL2, or mPAL3.

In some embodiments, the method of treatment, e.g., for PKU, and/or method of reducing hyperphenylalaninemia comprises administering a genetically engineered bacterium comprising four copies of a gene encoding PAL, e.g., PAL1, PAL3, mutant PAL, e.g., mPAL1, mPAL2, or mPAL3.

In some embodiments, the method of treatment, e.g., for PKU, and/or method of reducing hyperphenylalaninemia comprises administering a genetically engineered bacterium comprising five copies of a gene encoding PAL, e.g., PAL1, PAL3, mutant PAL, e.g., mPAL1, mPAL2, or mPAL3.

In some embodiments, the method of treatment, e.g., for PKU, and/or method of reducing hyperphenylalaninemia comprises administering a genetically engineered bacterium comprising six or more copies of a gene encoding PAL, e.g., PAL1, PAL3, mutant PAL, e.g., mPAL1, mPAL2, or mPAL3.

In some embodiments, at least one copy of the PAL gene is operably linked to an inducible promoter. In some embodiments, all copies of the PAL gene are operably linked to an inducible promoter. In some embodiments, at least one copy of the PAL gene is operably linked to an arabinose-inducible promoter. In some embodiments, at least one copy of the PAL gene is operably linked to an IPTG-inducible promoter. In some embodiments, at least one copy of the PAL gene is operably linked to a synthetic inducible promoter, e.g., Ptac. In some embodiments, at least one copy of the PAL gene is operably linked to an oxygen level-dependent promoter. In some embodiments, all copies of the PAL gene are operably linked to an IPTG-inducible promoter. The one or more copies of the PAL gene, e.g., PAL1, PAL3, mPAL1, mPAL2, or mPAL3, may be on a plasmid or integrated into the chromosome.

In some embodiments, the method of treatment, e.g., for PKU, and/or method of reducing hyperphenylalaninemia comprises administering a genetically engineered bacterium comprising PAL and further comprising one, two, three, four, five, six or more copies of a gene encoding LAAD. In some embodiments, at least one copy of the LAAD gene is operably linked to an inducible promoter, e.g., a synthetic inducible promoter. In some embodiments, all copies of the LAAD gene are operably linked to an inducible promoter. In some embodiments, at least one copy of the LAAD gene is operably linked to an arabinose-inducible promoter. In some embodiments, at least one copy of the LAAD gene is operably linked to an IPTG-inducible promoter, e.g., Ptac. The one or more copies of the LAAD gene may be on a plasmid or integrated into the chromosome.

In some embodiments, the method of treatment, e.g., for PKU, and/or method of reducing hyperphenylalaninemia comprises administering a genetically engineered bacterium comprising PAL and further comprising one, two, three, four, five, six or more copies of a gene encoding a phenylalanine transporter, e.g., pheP. In some embodiments, at least one copy of the phenylalanine transporter, e.g., pheP, gene is operably linked to an inducible promoter. In some embodiments, all copies of the phenylalanine transporter, e.g., pheP, gene are operably linked to an inducible promoter. In some embodiments, at least one copy of the phenylalanine transporter, e.g., pheP, gene is operably linked to an arabinose-inducible promoter. In some embodiments, at least one copy of the phenylalanine transporter, e.g., pheP, gene is operably linked to an IPTG-inducible promoter. In some embodiments, at least one copy of the phenylalanine transporter, e.g., pheP, gene is operably linked to a synthetic inducible promoter, e.g., Ptac. In some embodiments, at least one copy of the phenylalanine transporter, e.g., pheP, gene is operably linked to an oxygen level-dependent promoter. The one or more copies of the phenylalanine transporter, e.g., pheP, gene may be on a plasmid or integrated into the chromosome.

In some embodiments, the method of treatment, e.g., for PKU, and/or method of reducing hyperphenylalaninemia comprises administering a genetically engineered bacterium comprising four or five copies of a gene encoding PAL, e.g., PAL1, PAL3, mutant PAL, e.g., mPAL1, mPAL2, or mPAL3; one copy of a gene encoding LAAD operably linked to a promoter; and one copy of a gene encoding a phenylalanine transporter, e.g., pheP, operably linked to a promoter. In some embodiments, the method of treatment and/or method of reducing hyperphenylalaninemia comprises administering a genetically engineered bacterium comprising four or five copies of a gene encoding PAL, e.g., PAL1, PAL3, mutant PAL, e.g., mPAL1, mPAL2, or mPAL3 integrated into the chromosome; one copy of a gene encoding LAAD operably linked to a promoter; and one copy of a gene encoding a phenylalanine transporter, e.g., pheP, operably linked to a promoter.

In some embodiments, the method of treatment, e.g., for PKU, and/or method of reducing hyperphenylalaninemia comprises administering a genetically engineered bacterium comprising four or five copies of a gene encoding PAL, e.g., PAL1, PAL3, mutant PAL, e.g., mPAL1, mPAL2, or mPAL3, wherein one, two, three, four or all copies of the PAL gene is operably linked to an IPTG-inducible promoter; one copy of a gene encoding LAAD operably linked to a promoter; and one copy of a gene encoding a phenylalanine transporter, e.g., pheP, operably linked to a promoter (e.g., an IPTG-inducible promoter). In some embodiments, the method of treatment and/or method of reducing hyperphenylalaninemia comprises administering a genetically engineered bacterium comprising four or five copies of a gene encoding PAL, PAL1, PAL3, e.g., mutant PAL, e.g., mPAL1, mPAL2, or mPAL3, integrated into the chromosome and wherein one, two, three, four or all copies of the PAL gene is operably linked to an IPTG-inducible promoter; one copy of a gene encoding LAAD operably linked to a promoter; and one copy of a gene encoding a phenylalanine transporter, e.g., pheP, operably linked to a promoter (e.g., an IPTG-inducible promoter).

In some embodiments, the method of treatment, e.g., for PKU, and/or method of reducing hyperphenylalaninemia comprises administering a genetically engineered bacterium comprising four or five copies of a gene encoding PAL, e.g., PAL1, PAL3, mutant PAL, e.g., mPAL1, mPAL2, or mPAL3, wherein each copy of the PAL gene is operably linked to an IPTG-inducible promoter; one copy of a gene encoding LAAD operably linked to a promoter; and one copy of a gene encoding a phenylalanine transporter, e.g., pheP, operably linked to a promoter (e.g., an IPTG-inducible promoter). In some embodiments, the method of treatment and/or method of reducing hyperphenylalaninemia comprises administering a genetically engineered bacterium comprising four or five copies of a gene encoding PAL, e.g., PAL1, PAL3, mutant PAL, e.g., mPAL1, mPAL2, or mPAL3, integrated into the chromosome and wherein each copy of the PAL gene is operably linked to an IPTG-inducible promoter; one copy of a gene encoding LAAD operably linked to a promoter; and one copy of a gene encoding a phenylalanine transporter, e.g., pheP, operably linked to a promoter (e.g., an IPTG-inducible promoter).

In some embodiments, the genetically engineered bacterium comprises one or more of the following features: one or more heterologous genes encoding a phenylalanine ammonia lyase (PAL) operably linked to an IPTG inducible promoter, wherein the one or more PAL genes encode a polypeptide that has at least 90%, 91%, 92%, 93%, 94%, 95%, 96%, 97%, 98%, or 99% identity to, comprises, or consists of any one of SEQ ID NO: 500 or 503, one or more heterologous genes encoding a phenylalanine transporter operably linked to an IPTG inducible promoter, wherein the one or more phenylalanine transporter genes encode a polypeptide that has at least 90%, 91%, 92%, 93%, 94%, 95%, 96%, 97%, 98%, or 99% identity to, comprises, or consists of any one of SEQ ID NO: 509, one or more heterologous genes encoding a L-amino acid deaminase (LAAD) operably linked to an arabinose-inducible promoter, wherein the one or more LAAD genes encode a polypeptide that has at least 90%, 91%, 92%, 93%, 94%, 95%, 96%, 97%, 98%, or 99% identity to, comprises, or consists of any one of SEQ ID NO: 510, a phage deletion, a ΔdapA auxotrophy, and optionally a deletion of an endogenous pks island (also referred to herein as “colibactin island”).

In some embodiments, the genetically engineered bacterium comprises one or more of the following features: four copies of a heterologous gene encoding a phenylalanine ammonia lyase (PAL) operably linked to an IPTG inducible promoter, wherein the PAL genes encode a polypeptide that has at least 90%, 91%, 92%, 93%, 94%, 95%, 96%, 97%, 98%, or 99% identity to, comprises, or consists of any one of SEQ ID NO: 500 or 503, wherein the four copies of the PAL gene are integrated into the bacterial chromosome, one copy of a heterologous gene encoding a phenylalanine transporter operably linked to an IPTG inducible promoter, wherein the phenylalanine transporter gene encodes a polypeptide that has at least 90%, 91%, 92%, 93%, 94%, 95%, 96%, 97%, 98%, or 99% identity to, comprises, or consists of any one of SEQ ID NO: 509, wherein the phenylalanine transporter gene is integrated into the bacterial chromosome, one copy of a heterologous gene encoding a L-amino acid deaminase (LAAD) operably linked to an arabinose-inducible promoter, wherein the LAAD gene encodes a polypeptide that has at least 90%, 91%, 92%, 93%, 94%, 95%, 96%, 97%, 98%, or 99% identity to, comprises, or consists of any one of SEQ ID NO: 510, wherein the LAAD gene is integrated into the bacterial chromosomea phage deletion, and a ΔdapA auxotrophy, and optionally a deletion of an endogenous pks island.

In some embodiments, the method of treatment, e.g., for PKU, and/or method of reducing hyperphenylalaninemia comprises administering a genetically engineered bacterium further comprising one or more phage gene mutations that renders the phage genome(s) defective, e.g., such that lytic phage is not produced, and is optionally a dapA auxotroph.

In some embodiments, the method of treatment, e.g., for PKU, and/or method of reducing hyperphenylalaninemia comprises administering to a subject the genetically engineered bacterium SYNB1618 described herein. See, e.g., PCT/US2016/032562, PCT/US2016/062369, PCT/US2018/038840, the contents of which are hereby incorporated in their entireties.

In some embodiments, the method of treatment, e.g., for PKU, and/or method of reducing hyperphenylalaninemia comprises administering to a subject the genetically engineered bacterium SYNB1934 described herein. See, e.g., PCT/US2021/023003, PCT/US2021/063976, U.S. 63/132,627, the contents of which are hereby incorporated in their entireties.

In some embodiments, the method of treatment, e.g., for PKU, and/or method of reducing hyperphenylalaninemia comprises administering to a subject the bacterium described herein alone or in combination with one or more additional therapeutic agents. The additional therapeutic agent may be capable of stomach buffering. The additional therapeutic agent may be selected from a proton pump inhibitor (PPI), an H2 agonist, or an antiemetic, e.g., esomeprazole, omeprazole, lansoprazole, rabeprazole, pantoprazole, dexlansoprazole, Zegerid, or ranitidine, axid, pepcid, or tagamet.

The additional therapeutic may be an antiemetic medication. Non-limiting examples include diphenhydramine (Benadryl), meclizine (Bonine), promethazine (Phenergan), dimenhydrinate (Dramamine, Gravol), combination of sodium citrate, dextrose, and fructose (Nauzene), combination of orthophosphoric acid, fructose, and glucose (Emetrol), bismuth subsalicylate (Pepto Biμmol), dimenhydrinate (Dramamine, Gravol), prochlorperazine (Compazine), promethazine, vitamin B6, dexamethasone (Decadron), droperidol (Inapsine), granisetron (Kytril), metoclopramide (Reglan), ondansetron (Zofran), aprepitant (Emend), dexamethasone (DexPak), dolasetron (Anzemet), palonosetron (Aloxi), prochlorperazine (Compazine), and rolapitant (Varubi).

The additional therapeutic agent may be administered before, after, or concurrently with administration of the bacterium. For example, a proton pump inhibitor may be administered e.g., once daily, prior, e.g., 60 to 90 minutes prior to a meal and the genetically engineered bacteria may be administered immediately after a meal, e.g., one to three times daily.

In some embodiments, the method of treatment, e.g., for PKU, and/or method of reducing hyperphenylalaninemia comprises administering to a subject a dose of about 1×10¹², about 2×10¹², about 3×10¹², about 4×10¹², about 5×10¹², about 6×10¹², about 7×10¹², about 8×10¹², or about 9×10¹² of the bacterial cells described herein, as determined by live cell counting. In a specific embodiment, the method of treatment, e.g., for PKU, and/or method of reducing hyperphenylalaninemia comprises administering to a subject a dose between 1×10¹² and 2×10¹² of the bacterial cells described herein as determined by live cell counting.

In some embodiments, the method of treatment, e.g., for PKU, and/or method of reducing hyperphenylalaninemia comprises administering to a subject a dose of about 1×10¹¹, about 2×10¹¹, about 3×10¹¹, about 4×10¹¹, about 5×10¹¹, about 6×10¹¹, about 7×10¹¹, about 8×10¹¹, or about 9×10¹¹ of the bacteria, e.g., bacterial cells, described herein as determined by live cell counting.

In some embodiments, the method of treatment, e.g., for PKU, and/or method of reducing hyperphenylalaninemia comprises administering to a subject a dose of about 1×10¹², about 1.1×10¹², about 1.2×10¹², about 1.3×10¹², about 1.4×10¹², about 1.5×10¹², about 1.6×10¹², about 1.7×10¹², about 1.8×10¹², about 1.9×10¹², about 2×10¹², about 2.1×10¹², about 2.2×10¹², about 2.3×10¹², about 2.4×10¹², about 2.5×10¹², about 2.6×10¹², about 2.7×10¹², about 2.8×10¹², about 2.9×10¹², or about 3×10¹² of the bacteria, e.g., bacterial cells described herein as determined by live cell counting. In some embodiments, the method of treatment, e.g., for PKU, and/or method of reducing hyperphenylalaninemia comprises administering to a subject a dose described herein, e.g., 1×10¹¹, 3×10¹¹, 1×10¹², or 2×10¹² live cells, once per day (QD), twice per day (BID), or three times per day (TID). In some embodiments, the dose may be administered immediately after a meal.

Administering the bacteria to the subject may result in gastrointestinal-related adverse events, e.g., bloating, nausea, and/or change in bowel habits. In some embodiments, the adverse effects can be reduced by using a dose ramp. Thus, in some embodiments, the method of treatment, e.g., for PKU, and/or method of reducing hyperphenylalaninemia comprises administering to a subject escalating dose levels (dose ramp) of the bacteria described herein to obtain an individually titrated dose (iTD). In some embodiments, dose is escalated based on tolerability. In some embodiments, the dose ramp (e.g., a gradual and extended dose ramp) improves the dosing experience and helps more patients benefit from the treatment.

In some embodiments, a dose is considered intolerable is a subject experiences Grade 3 or more severe adverse events or Grade 2 gastrointestinal adverse events that do not improve with continued dosing at the same dose over time. In some embodiments, the method comprises administering to a subject a first dose of the bacteria for a first dosing interval (e.g., 2, 3, or 4 weeks), followed by a second dose of the bacteria that is higher than the first dose for a second dosing interval (e.g., 2, 3, or 4 weeks). In some embodiments, this is followed by a third dose of the bacteria that is higher than the second dose for a third dosing interval (e.g., 2, 3, or 4 weeks). In some embodiments, where the subject tolerates the first dose during the first dosing interval, e.g., after 2, 3, or 4 weeks according to the metrics described herein, the subject escalates to the second dose for a second dosing interval. In some embodiments, where the subject does not tolerate the second dose during the second dosing interval, e.g., after 2, 3, or 4 weeks according to the metrics described herein, the subject de-escalates to the first dose. In some embodiments, where the subject tolerates the second dose during the second dosing interval, e.g., after 2, 3, or 4 weeks according to the metrics described herein, the subject escalates to the third dose for the third dosing interval. In some embodiments, where the subject does not tolerate the third dose during the third dosing interval, e.g., after 2, 3, or 4 weeks according to the metrics described herein, the subject de-escalates to the second dose.

In some embodiments, where the subject tolerates the first dose during a first dosing interval of 3 weeks according to the metrics described herein, the subject escalates to the second dose for a second dosing interval of 3 weeks. In some embodiments, where the subject does not tolerate the second dose during the second dosing interval of 3 weeks according to the metrics described herein, the subject de-escalates to the first dose. In some embodiments, where the subject tolerates the second dose during the second dosing interval of 3 weeks according to the metrics described herein, the subject escalates to the third dose for a third dosing interval of 3 weeks. In some embodiments, where the subject does not tolerate the third dose during the third dosing interval of 3 weeks according to the metrics described herein, the subject de-escalates to the second dose.

In some embodiments, during each dosing interval (e.g., the first dosing interval, the second dosing interval, the third dosing interval), the bacteria are first administered once per day (QD) for a set amount of days, then twice per day (BID) for a set amount of days, and then three times per day (TID) for the remainder of the dosing interval. In some embodiments, a patient may initially take a half-dose, e.g., by mixing the entire contents of a sachet in which the bacteria are provided and then discarding half, e.g., for one or two days. In some embodiments, a patient may initially take a one third of a whole dose, e.g., by mixing the entire contents of a sachet in which the bacteria are provided and then discarding two thirds, e.g., for one or two days.

In specific embodiments, during each dosing interval (e.g., the first dosing interval, the second dosing interval, the third dosing interval), the bacteria are administered once per day (QD) for two days, twice per day (BID) for 2 days, and then three times per day (TID) for the remainder of the dosing interval.

The range, variability, extent and level of the Phe response may be assessed prior to and/or and after each dosing interval. Additionally, the Phe response may also be measured within a particular dosing interval before and after QD, BID, and TID administration levels. Alternatively, weekly Phe assessments may be conducted.

Similarly, the range, variability, extent and level of tyrosine may be assessed prior to and/or and after each dosing interval, and also within a particular dosing interval at each dosing level (i.e., before and after QD, BID, and TID administration levels). Alternatively, weekly tyrosine assessments may be conducted.

In some embodiments, the first dose is about 1×10¹¹, about 2×10¹¹, about 3×10¹¹, about 4×10¹¹, or about 5×10¹¹ of the bacterial cells described herein (e.g., SYNB1934) as determined by live cell counting. In some embodiments, the first dose is about 3×10¹¹ of the bacterial cells described herein (e.g., SYNB1934) as determined by live cell counting. In some embodiments, the first dose is 3×10¹¹ of the bacterial cells described herein (e.g., SYNB1934) as determined by live cell counting. In some embodiments, the second dose is about 4×10¹¹, about 5×10¹¹, about 6×10¹¹, about 7×10¹¹, or about 8×10¹¹ of the bacterial cells described herein (e.g., SYNB1934) as determined by live cell counting. In some embodiments, the second dose is about 6×10¹¹ of the bacterial cells described herein (e.g., SYNB1934) as determined by live cell counting. In some embodiments, the second dose is 6×10¹¹ of the bacterial cells described herein (e.g., SYNB1934) as determined by live cell counting. In some embodiments, the third dose is about 8×10¹¹, about 9×10¹¹, about 1×10¹², about 1.1×10¹², or about 1.2×10¹² of the bacteria, e.g., bacterial cells, described herein (e.g., SYNB1934) as determined by live cell counting. In some embodiments, the third dose is about 1×10¹² of the bacterial cells described herein (e.g., SYNB1934) as determined by live cell counting. In some embodiments, the third dose is 1×10¹² of the bacterial cells described herein (e.g., SYNB1934) as determined by live cell counting. In some embodiments, a subject is administered a first dose of 3×10¹¹, a second dose of 6×10¹¹, and a third dose of 1×10¹² of the bacterial cells described herein (e.g., SYNB1934) as determined by live cell counting.

In certain embodiments, the method of treatment, e.g., for PKU, and/or method of reducing hyperphenylalaninemia may comprise genetically engineered bacteria that are capable of metabolizing phenylalanine in the diet or gut-resident free phenylalanine present in the small intestine. Studies have shown that pancreatic and other glandular secretions into the intestine contain high levels of proteins, enzymes, and polypeptides, and that the amino acids produced as a result of their catabolism are reabsorbed back into the blood in a process known as “enterorecirculation” (Chang, 2007; Sarkissian et al., 1999). Thus, high intestinal levels of phenylalanine may be partially independent of food intake and are available for breakdown by a phenylalanine metabolizing enzyme, e.g., PAL, e.g., as expressed in a genetically engineered bacterium disclosed herein. In some embodiments, the genetically engineered bacteria and dietary protein are delivered after a period of fasting or phenylalanine-restricted dieting. In some embodiments, the genetically engineered bacteria may be capable of metabolizing phenylalanine enterorecirculating from the blood. In these embodiments, the genetically engineered bacteria need not be delivered simultaneously with dietary protein. A phenylalanine gradient is generated, e.g., from blood to gut, where the genetically engineered bacteria metabolize phenylalanine. A patient suffering from hyperphenylalaninemia may be able to resume a substantially normal diet, or a diet that is less restrictive than the stringent low-phe diet recommended for example to reach/maintain a target Phe of <360 umol/L. In some embodiments, the genetically engineered bacteria are delivered simultaneously or right after dietary protein. In other embodiments, the genetically engineered bacteria are not delivered simultaneously with dietary protein.

In some embodiments, the method of treatment, e.g., for PKU, comprises measuring baseline phenylalanine dietary intake prior to administration of the genetically engineered bacteria. In some embodiments, the baseline measurement is made in a fasted state, e.g., prior to a meal, e.g., in a subject having phenylketonuria. In some embodiments, the baseline phenylalanine dietary intake is recorded for 10, 9, 8, 7, 6, 5, 4, 3, 2, or 1 days prior to administration of the genetically engineered bacteria, e.g., for 3 days. In some embodiments, the method of treatment comprises measuring phenylalanine at various time points while a subject is being treated with the genetically engineered bacteria. Dietary phenylalanine intake during treatment is determined using the baseline measurement, e.g., dietary phenylalanine intake may be within ±5%, ±10%, ±15%, or ±20% of the subject's baseline phenylalanine intake. Baseline dietary deviations of phenylalanine may be <10% during diet run-in or <25% during diet run-in. A subject may record a 3 day dietary intake regularly, i.e., prior and/or during the administration period. In some instances, dietary intake may be recorded daily during the administration period. In some embodiments, the period of time at which a measurement is taken is 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 or more days after administration of the genetically engineered bacterium.

The method may comprise administering a pharmaceutical composition with at least one genetically engineered species, strain, or subtype of bacteria described herein, and administering the pharmaceutical composition to a subject in a therapeutically effective amount. In some embodiments, the genetically engineered bacteria are administered orally, e.g., in a liquid suspension. In some embodiments, the genetically engineered bacteria are lyophilized and administered orally, e.g., provided in a sachet. In some embodiments, the genetically engineered bacteria are administered via a feeding tube or gastric shunt. In some embodiments, the genetically engineered bacteria are administered rectally, e.g., by enema. In some embodiments, the genetically engineered bacteria are administered topically, intraintestinally, intrajejunally, intraduodenally, intraileally, and/or intracolically.

In certain embodiments, the methods provided herein are capable of reducing phenylalanine levels in a subject. In some embodiments, the methods of the present disclosure reduce the phenylalanine levels in a subject by at least 10%, at least 20%, at least 25%, at least 30%, at least 40%, at least 50%, at least 60%, at least 70%, at least 75%, at least 80%, at least 85%, at least 90%, at least 95%, or more as compared to levels in an untreated or control subject, e.g., in the subject after a suitable period of time after administration of the genetically engineered bacterium. In some embodiments, reduction is measured by comparing the phenylalanine level in a subject before and after administration of the pharmaceutical composition. In some embodiments, the methods of the disclosure reduce blood phenylalanine levels in a subject by at least 5%, at least 10%, at least 20%, at least 25%, at least 30%, at least 40%, at least 50%, at least 60%, at least 70%, at least 75%, at least 80%, at least 85%, at least 90%, at least 95%, or more as compared to the baseline measurement prior to administration of the bacteria. In some embodiments, the method of treating or ameliorating hyperphenylalaninemia allows one or more symptoms of the condition or disorder to improve by at least 10%, at least 20%, at least 30%, at least 40%, at least 50%, at least 60%, at least 70%, at least 80%, at least 90%, at least 95%, or more.

In some embodiments, the methods disclosed herein reduce blood phenylalanine levels to at least >1,200 μmol/L, at least >600 μmol/L, at least >360 μmol/L, at least >180 μmol/L, >120 μmol/L. In some embodiments, the methods disclosed herein reduce blood phenylalanine levels to at least >1,200 μmol/L, at least 1200 μmol/L-600 μmol/L, at least 600 μmol/L-360 μmol/L, at least 360 μmol/L-180 μmol/L, at least 180 μmol/L to 120 μmol/L.

In certain embodiments, the methods provided herein are capable of reducing phenylalanine levels in a subject, thereby allowing the subject to consume increased amounts of protein after administration of the bacteria while maintaining or lowering blood phenylalanine as compared to before administration of the genetically engineered bacterium. In some embodiments, the subject is able to consume at least 10%, at least 20%, at least 25%, at least 30%, at least 40%, at least 50%, at least 60%, at least 70%, at least 75%, at least 80%, at least 85%, at least 90%, at least 95%, or more protein while maintaining or lowering blood phenylalanine as compared to before administration of the genetically engineered bacterium. In some embodiments, the subject is able to consume at least 1 g, at least 2 g, at least 3 g, at least 4 g, at least 5 g, at least 6 g, at least 7 g, at least 8 g, at least 9 g, or at least 10 g more protein while maintaining or lowering blood phenylalanine as compared to before administration of the genetically engineered bacterium. In some embodiments, the subject is able to consume at least 10 g, at least 11 g, at least 12 g, at least 13 g, at least 14 g, at least 15 g, at least 16 g, at least 17 g, at least 18 g, at least 19 g, or at least 20 g or more additional protein while maintaining or lowering blood phenylalanine as compared to before administration of the genetically engineered bacterium.

On average, 1000 mg of protein contains about 50 mg of Phe. In some embodiments, the subject is able to consume at least 50 mg, at least 100 mg, at least 150 mg, at least 200 mg, at least 250 mg, at least 300 mg, at least 350 mg, at least 400 mg, at least 450 mg, or at least 500 mg more Phe daily while maintaining or lowering blood phenylalanine as compared to before administration of the genetically engineered bacterium. In some embodiments, the subject is able to consume at least 500 mg, at least 550 mg, at least 600 mg, at least 650 mg, at least 700 mg, at least 750 mg, at least 800 mg, at least 850 mg, at least 900 mg, at least 950 mg, or at least 1000 mg more Phe daily while maintaining or lowering blood phenylalanine as compared to before administration of the genetically engineered bacterium. In some embodiments, the subject is able to increase protein intake by at least 0.1 g/kg/day, at least 0.2 g/kg/day, or at least 0.3 g/kg/day as compared to before administration of the genetically engineered bacterium. Exemplary, non-limiting phenylalanine intake adjustments are provided in Table 7. See, e.g., Muntau et al., 2017; Trefz et al., 2008.

TABLE 7
Exemplary phenylalanine intake adjustments

| Mean Phe | | |
|---|---|---|
| concentration | | Corresponding Protein |
| (μmol/L) | Phe intake adjustment | intake adjustment |
| 0-180 | Increase by 15 mg/kg/day | Increase by 0.3 g/kg/day |
| 181-240 | Increase by 10 mg/kg/day | Increase by 0.2 g/kg/day |
| 241-300 | Increase by 5 mg/kg/day | Increase by 0.1 g/kg/day |

In some embodiments, the subject is able to consume at least 1 mg/kg/day, at least 2 mg/kg/day, at least 3 mg/kg/day, at least 4 mg/kg/day, at least 5 mg/kg/day, at least 6 mg/kg/day, at least 7 mg/kg/day, at least 8 mg/kg/day, at least 9 mg/kg/day, at least 10 mg/kg/day, at least 11 mg/kg/day, at least 12 mg/kg/day, at least 13 mg/kg/day, at least 14 mg/kg/day, at least 15 mg/kg/day, at least 16 mg/kg/day, at least 17 mg/kg/day, at least 18 mg/kg/day, at least 19 mg/kg/day, at least 20 mg/kg/day more Phe daily while maintaining or lowering blood phenylalanine as compared to before administration of the genetically engineered bacterium.

In some embodiments, the subject is able to consume at least 0.05 g/kg/day, at least 0.1 g/kg/day, at least 0.2 g/kg/day, at least 0.3 g/kg/day, at least 0.4 g/kg/day, or more protein daily while maintaining or lowering blood phenylalanine as compared to before administration of the genetically engineered bacterium.

For example, upon administration of the engineered bacteria described herein, a subject may achieve a mean Phe concentration of 0-300 μmol/L, e.g., at the time of their first measurement post initiation of treatment, and then increase Phe intake by 5 mg/kg/day or increase protein intake by 0.1 g/kg/day.

For example, upon administration of the engineered bacteria described herein, a subject may achieve a mean Phe concentration of 0-180 μmol/L and then increase Phe intake by 15 mg/kg/day or increase protein intake by 0.1 g/kg/day.

For example, upon administration of the engineered bacteria described herein, a subject may achieve a mean Phe concentration of 181-240 μmol/L and then increase Phe intake by 10 mg/kg/day or increase protein intake by 0.2 g/kg/day.

For example, upon administration of the engineered bacteria described herein, a subject may achieve a mean Phe concentration of 241-300 μmol/L and then increase Phe intake by 5 mg/kg/day or increase protein intake by 0.1 g/kg/day.

In some embodiments, the disclosure provides a method for measuring activity of a genetically engineered bacterium of the disclosure in vivo by administering to a subject, e.g., a mammalian subject, said bacterium, and measuring the amount of blood Phe lowering or blood Phe levels in the subject as a measure of PAL activity. In some embodiments, the disclosure provides a method for monitoring the therapeutic activity of a genetically engineered bacterium of the disclosure by administering to a subject, e.g., a mammalian subject, said bacterium and measuring the amount of blood Phe lowering or blood Phe levels in the subject as a measure of therapeutic activity. In some embodiments, the disclosure provides a method for adjusting the dosage of a genetically engineered bacterium of the disclosure by administering to a subject, e.g., a mammalian subject, said bacterium, measuring the amount of blood Phe lowering or blood Phe levels in the subject to determine strain activity, and adjusting (e.g., increasing or decreasing) the dosage of the bacterium to increase or decrease blood Phe lowering or blood Phe levels in the subject. In some embodiments, the disclosure provides a method for adjusting the protein intake and/or diet of a subject having hyperphenylalaninemia comprising administering to the subject a genetically engineered bacterium of the disclosure, measuring the amount of blood Phe consumed in the subject, and adjusting (e.g., increasing or decreasing) the protein intake or otherwise adjusting the diet of the subject to increase or decrease blood Phe consumption or blood Phe levels in the subject. In some embodiments, the disclosure provides a method for confirming adherence to a protein intake and/or diet regimen of a subject having hyperphenylalaninemia comprising administering to the subject a bacterium of the disclosure, measuring the amount of blood Phe lowering in the subject or blood Phe levels in the subject.

In some embodiments of the methods disclosed herein, both blood phenylalanine levels are monitored in a subject. In some embodiments, blood phenylalanine levels measured at multiple time points, to determine the rate of phenylalanine breakdown.

In some embodiments, blood phenylalanine measurements, are used evaluate safety in animal models and human subjects. In some embodiments, blood phenylalanine measurements, are used in the evaluation of dose-response and optimal regimen for the desired pharmacologic effect and safety. In some embodiments, blood phenylalanine measurements are used as surrogate endpoint for efficacy and/or toxicity. In some embodiments, blood phenylalanine measurements are used to predict patients' response to a regimen comprising a therapeutic strain. In some embodiments, blood phenylalanine measurements, are used for the identification of certain patient populations that are more likely to respond to the drug therapy. In some embodiments, blood phenylalanine measurements are used to avoid specific adverse events. In some embodiments, blood phenylalanine measurements are useful for patient selection. In some embodiments, with blood phenylalanine measurements, are used as one method for adjusting protein intake/diet of PKU patient on a regimen which includes the administration of a therapeutic PKU strain expressing PAL.

Trans-cinnamate (or TCA) produced from phenylalanine specifically by PAL, is a measure of PAL activity. In some embodiments, the methods of administration described herein increase levels of trans-cinnamate, e.g., in blood or urine. Prior to administration of the genetically engineered bacteria, cinnamate is not detectable. Accordingly, cinnamate may be used as an alternative biomarker for strain activity. In some embodiments, the methods herein increase trans-cinnamate levels to detectable levels post administration of the genetically engineered bacteria.

Hippurate is a breakdown product of TCA produced by several naturally occurring enzymes and is normally present in human urine. It is also the end product of metabolism of phenylalanine via the PAL pathway. Phenylalanine ammonia lyase mediates the conversion of phenylalanine to cinnamate. When cinnamate is produced in the small intestine, it is absorbed and quickly converted to hippurate in the liver and excreted in the urine (Hoskins J A and Gray Phenylalanine ammonia lyase in the management of phenylketonuria: the relationship between ingested cinnamate and urinary hippurate in humans. J Res Commun Chem Pathol Pharmacol. 1982 February; 35(2):275-82). Phenylalanine is converted to hippurate in a 1:1 ratio, i.e., 1 mole of Phe is converted into 1 mol of hippurate. Thus, changes in urinary hippurate levels can be used as a non-invasive measure of the effect of therapies that utilize this mechanism. Hippurate levels in the subject may be measured in a biological sample, such as blood, serum, plasma, urine, or fecal matter.

In some embodiments, the activity (e.g., phenylalanine degrading activity) of genetically engineered microorganism expressing PAL, e.g., mutant PAL, can be detected in the urine of a mammalian subject, e.g., an animal model or a human, by measuring the amounts of hippurate produced and the rate of its accumulation.

In this section, the term “PAL-based drug” refers to any drug, polypeptide, biologic, or treatment regimen that has PAL activity, for example, a composition comprising a microorganism of the present disclosure, e.g., microorganism encoding PAL and optionally PheP transporter. In some embodiments, the disclosure provides a method for measuring PAL activity in vivo by administering to a subject, e.g., a mammalian subject, a PAL-based drug and measuring the amount of a suitable biomarker.

Hippuric acid thus has the potential to function as a biomarker allowing monitoring of dietary adherence and treatment effect in patients receiving PAL-based regimens. It can be used as an adjunct to measurement of blood Phe levels in the management of patients and because it is a urinary biomarker, it can have advantages particularly in children to adjust protein intake—which can be challenging as needs vary based on growth.

In some embodiments, the methods of administering increase levels of hippurate production. In some embodiments, the methods may include administration of the compositions of the invention, leading to an increase hippurate of at least 2-fold, at least 3-fold, at least 4-fold, at least 5-fold, at least 6-fold, at least 7-fold, at least 8-fold, at least 9-fold, or at least 10-fold of the subject's urine hippurate levels prior to treatment.

In some embodiments, the disclosure provides a method for monitoring the therapeutic activity of a PAL-based drug by administering to a subject, e.g., a mammalian subject, the PAL-based drug and measuring the amount of hippurate produced in the subject as a measure of PAL therapeutic activity. In some embodiments, the disclosure provides a method for adjusting the dosage of a PAL-based drug by administering to a subject, e.g., a mammalian subject, the PAL-based drug, measuring the amount of hippurate produced in the subject to determine PAL activity, and adjusting (e.g., increasing or decreasing) the dosage of the drug to increase or decrease the PAL activity in the subject. In some embodiments, the disclosure provides a method for adjusting the protein intake and/or diet of a subject having hyperphenylalaninemia comprising administering to the subject a PAL-based drug, measuring the amount of hippurate produced in the subject, and adjusting (e.g., increasing or decreasing) the protein intake or otherwise adjusting the diet of the subject to increase or decrease the PAL activity in the subject. In some embodiments, the disclosure provides a method for confirming adherence to a protein intake and/or diet regimen of a subject having hyperphenylalaninemia comprising administering to the subject a PAL-based drug, measuring the amount of hippurate produced in the subject, and measuring PAL activity in the subject.

In some embodiments of the methods disclosed herein, both blood phenylalanine levels and urine hippurate levels are monitored in a subject. In some embodiments, blood phenylalanine and hippurate in the urine are measured at multiple time points, to determine the rate of phenylalanine breakdown. In some embodiments, hippurate levels in the urine are used evaluate PAL activity or strain activity in animal models.

In some embodiments, hippuric acid measurements in the urine, alone or in combination with blood phenylalanine measurements, are used to the strain prove mechanism of action. In some embodiments, hippuric acid measurements in the urine, alone or in combination with blood phenylalanine measurements, are used as a tool to differentiate between PAL and LAAD activity in a strain, and allow to determine the contribution of each enzyme to the overall strain activity.

In some embodiments, hippuric acid measurements in the urine, alone or in combination with blood phenylalanine measurements, are used evaluate safety in animal models and human subjects. In some embodiments, hippuric acid measurements in the urine, alone or in combination with blood phenylalanine measurements, are used in the evaluation of dose-response and optimal regimen for the desired pharmacologic effect and safety. In some embodiments, hippuric acid measurements in the urine, alone or in combination with blood phenylalanine measurements, are used as surrogate endpoint for efficacy and/or toxicity. In some embodiments, hippuric acid measurements in the urine, alone or in combination with blood phenylalanine measurements, are used to predict patients' response to a regimen comprising a therapeutic strain. In some embodiments, hippuric acid measurements in the urine, alone or in combination with blood phenylalanine measurements, are used for the identification of certain patient populations that are more likely to respond to the drug therapy. In some embodiments, hippuric acid measurements in the urine, alone or in combination with blood phenylalanine measurements, are used to avoid specific adverse events. In some embodiments, hippuric acid measurements in the urine, alone or in combination with blood phenylalanine measurements, are useful for patient selection.

In some embodiments, hippuric acid measurements in the urine, alone or in combination with blood phenylalanine measurements, are used as one method for adjusting protein intake/diet of PKU patient on a regimen which includes the administration of a therapeutic PKU strain expressing PAL.

In some embodiments, measurement of urine levels of hippuric acid, alone or in combination with blood phenylalanine measurements, is used to measure and/or monitor the activity of recombinant PAL. In some embodiments, measurement of urine levels of hippuric acid is used to measure and/or monitor the activity of recombinant pegylated PAL (Peg-PAL). In some embodiments, measurement of urine levels of hippuric acid, alone or in combination with blood phenylalanine measurements, is used to measure and/or monitor the activity of recombinant PAL administered in combination with a therapeutic strain as described herein.

In some embodiments, clinical safety markers may be measured. Non-limiting examples of clinical safety markers include physical examination, vital signs, and electrocardiogram (ECG). Other non-limiting examples include liver safety tests known in the art, e.g., serum aspartate transaminase (AST), alanine transaminase (ALT), alkaline phosphatase (ALP), gamma-glutamyl transferase (GGT), and bilirubin. Such biosafety markers also include renal safety tests, e.g., those known in the art, e.g., blood urea nitrogen (BUN), serum creatinine, glomerular filtration rate (GFR), creatinine clearance, serum electrolytes (sodium, potassium, chloride, and bicarbonate), and complete urine analysis (color, pH, specific gravity, glucose, proteins, ketone bodies, and microscopic exam for blood, leukocytes, casts), as well as Cystatin-c, β2-microglobulin, uric acid, clusterin, N-acetyl-beta-dglucosaminidase, neutrophil gelatinase-associated lipocalin (NGAL), N-acetyl-β-dglucosaminidase (NAG), and kidney injury molecule-1 (KIM-1). Other non-limiting examples include Hematology safety biomarkers known in the art, e.g., Complete blood count, total hemoglobin, hematocrit, red cell count, mean red cell volume, mean cell hemoglobin, red cell distribution width %, mean cell hemoglobin concentration, total white cell count, differential white cell count (Neutrophils, lymphocytes, basophils, eosinophils, and monocytes), and platelets. Other no-liming examples include bone safety markers known in the art, e.g., Serum calcium and inorganic phosphates. Other non-limiting examples include basic metabolic safety biomarkers known in the art, e.g., blood glucose, triglycerides (TG), total cholesterol, low density lipoprotein cholesterol (LDLc), and high density lipoprotein cholesterol (HDL-c). Other specific safety biomarkers known in the art include, e.g., serum immunoglobulin levels, C-reactive protein (CRP), fibrinogen, thyroid stimulating hormone (TSH), thyroxine, testosterone, insulin, lactate dehydrogenase (LDH), creatine kinase (CK) and its isoenzymes, cardiac troponin (cTn), and methemoglobin.

In some embodiments, urine D5-hippuric acid is measured following D5-Phe administration and dosing of the genetically engineered bacteria, e.g., over 3, 4, 5, 6, 7, 8, 9, 10, 11, 12, 13, 14, 15, 16, 17, 18, 19, 20, 21, 22, 23, or 24 hours and/or compared to a suitable control and may be used to evaluate efficacy and/or safety in a subject.

In some embodiments, clearance of the genetically engineered bacteria is measured, e.g., by qPCR, following dosing, and may be used to evaluate or safety and/or clearance in a subject.

In some embodiments, change from baseline in plasma Phe, plasma TCA area under the curve (AUC), and/or urinary HA Aet is measured following dosing of the genetically engineered bacteria, e.g., over 9, 10, 11, 12, 13, 14, 15, 16, 17, 18, 19, 20, 21, 22, 23, or 24 hours, e.g., during a tracer study, and may be used to evaluate efficacy and/or safety in a subject.

In some embodiments, change from baseline in plasma D5-Phe and/or plasma D5-TCA AUC is measured following dosing, e.g., over 3, 4, 5, 6, 7, or 8 hours, e.g., during a tracer study, and may be used to evaluate efficacy and/or safety in a subject.

In some embodiments, change from baseline in plasma D5-Phe is measured by D5-Phe AUC following dosing of the genetically engineered bacteria and D5-Phe administration, e.g., over 12, 16, 20, 24, 28, or 32 hours, and may be used to evaluate efficacy and/or safety in a subject, e.g., on day 10, 11, 12, 13, 14, 15, 16, 17, 18, 19, and/or 20. In some embodiments, the measurement is made at day 14.

In these embodiments, the dose of the genetically engineered bacteria, e.g., SYNB1618, SYNB1934, may be 1×10¹² live cells, 2×10¹² live cells, or 3×10¹², or 4×10¹² live cells. In these embodiments, the dose of the genetically engineered bacteria, e.g., SYNB1618, SYNB1934, may be 2×10¹² live cells.

In some embodiments, change from baseline in fasting levels of plasma Phe is measured, e.g., on day 7, day 14, day 21, and/or day 28 (e.g., day 29±3), and may be used to evaluate efficacy and/or safety in a subject.

In some embodiments, change from baseline in plasma TCA after a low Phe meal is measured, e.g., by TCA AUC, e.g., over 3, 4, 5, 6, 7, or 8 hours, e.g., on day 7, 8, 9, 10, 11, 12, 13, 14, 15, or 16, and may be used to evaluate efficacy in a subject.

In some embodiments, change from baseline in urine HA is measured, e.g., on day 7, 8, 9, 10, 11, 12, 13, 14, 15, or 16, and may be used to evaluate efficacy in a subject.

In some embodiments, change in suitable CANTAB item score(s) from baseline is measured, e.g., on day 10, 11, 12, 13, 14, 15, 16, 17, or 18, and may be used to evaluate efficacy and/or safety in a subject.

In some embodiments, the methods disclosed herein comprise administering the genetically engineered bacteria disclosed herein with labeled phenylalanine, e.g., D5-phenylalanine. In these embodiments, the symptom to be assessed may be labeled phenylalanine, e.g., D5-phenylalanine; labeled cinnamate, e.g., D5-TCA; and/or labeled hippurate, e.g., D5 HA. In these embodiments, the levels of labeled phenylalanine after administration of the genetically engineered bacteria is decreased by at least 10%, at least 20%, at least 30%, at least 40%, at least 50%, at least 60%, at least 70%, at least 80%, at least 90%, at least 95%, or more as compared to baseline before administration, e.g., fasted, e.g., prior to a meal. In these embodiments, the levels of labeled cinnamate and/or hippurate after administration of the genetically engineered bacteria are increased by at least 10%, at least 20%, at least 30%, at least 40%, at least 50%, at least 60%, at least 70%, at least 80%, at least 90%, at least 95%, or more as compared to baseline before administration, e.g., fasted, e.g., prior to a meal.

In some embodiments, the method comprises administering to a subject a dose of about 3×10¹¹ of the bacterial cells described herein, e.g., SYNB1618 or SYNB1934, as determined by live cell counting and achieving at least 10%, at least 15%, at least 20%, at least 25%, at least 30%, at least 35%, at least 40%, at least 45%, or at least 50% reduction in blood phenylalanine, e.g., as measured 7 or 14 days after administration of the bacteria. In some embodiments, the subject achieves at least 10% reduction in blood phenylalanine, e.g., as measured 7 or 14 days after administration of the bacteria. In some embodiments, the subject achieves at least 20% reduction in blood phenylalanine, e.g., as measured 7 or 14 days after administration of the bacteria. In some embodiments, the subject achieves at least 30% reduction in blood phenylalanine, e.g., as measured 7 or 14 days after administration of the bacteria. In some embodiments, the subject receives labeled phenylalanine, e.g., D5-phenylalanine, with the genetically engineered bacteria, and the labeled phenylalanine levels are decreased by at least 10%, at least 15%, at least 20%, at least 25%, at least 30%, at least 35%, at least 40%, at least 45%, or at least 50% as compared to baseline before administration. Labeled TCA may be detectable and increase proportionately with phenylalanine reduction.

In some embodiments, the method comprises administering to a subject a dose of about 6×10¹¹ of the bacterial cells described herein, e.g., SYNB1618 or SYNB1934, as determined by live cell counting and achieving at least 10%, at least 15%, at least 20%, at least 25%, at least 30%, at least 35%, at least 40%, at least 45%, or at least 50% reduction in blood phenylalanine, e.g., as measured 7 or 14 days after administration of the bacteria. In some embodiments, the subject achieves at least 10% reduction in blood phenylalanine, e.g., as measured 7 or 14 days after administration of the bacteria. In some embodiments, the subject achieves at least 20% reduction in blood phenylalanine, e.g., as measured 7 or 14 days after administration of the bacteria. In some embodiments, the subject achieves at least 30% reduction in blood phenylalanine, e.g., as measured 7 or 14 days after administration of the bacteria. In some embodiments, the subject receives labeled phenylalanine, e.g., D5-phenylalanine, with the genetically engineered bacteria, and the labeled phenylalanine levels are decreased by at least 10%, at least 15%, at least 20%, at least 25%, at least 30%, at least 35%, at least 40%, at least 45%, or at least 50% as compared to baseline before administration. Labeled TCA may be detectable and increase proportionately with phenylalanine reduction.

In some embodiments, the method comprises administering to a subject a dose of about 1×10¹² of the bacterial cells described herein, e.g., SYNB1618 or SYNB1934, as determined by live cell counting and achieving at least 10%, at least 15%, at least 20%, at least 25%, at least 30%, at least 35%, at least 40%, at least 45%, or at least 50% reduction in blood phenylalanine, e.g., as measured 7 or 14 days after administration of the bacteria. In some embodiments, the subject achieves at least 10% reduction in blood phenylalanine, e.g., as measured 7 or 14 days after administration of the bacteria. In some embodiments, the subject achieves at least 20% reduction in blood phenylalanine, e.g., as measured 7 or 14 days after administration of the bacteria. In some embodiments, the subject achieves at least 30% reduction in blood phenylalanine, e.g., as measured 7 or 14 days after administration of the bacteria. In some embodiments, the subject receives labeled phenylalanine, e.g., D5-phenylalanine, with the genetically engineered bacteria, and the labeled phenylalanine levels are decreased by at least 10%, 15%, 20%, 25%, 30%, 35%, 40%, 45%, or 50% as compared to baseline before administration. Labeled TCA may be detectable and increase proportionately with phenylalanine reduction.

In some embodiments, the method comprises administering to a subject a dose of about 2×10¹² of the bacterial cells described herein, e.g., SYNB1618 or SYNB1934, as determined by live cell counting and achieving at least 10%, at least 15%, at least 20%, at least 25%, at least 30%, at least 35%, at least 40%, at least 45%, or at least 50% reduction in blood phenylalanine, e.g., as measured 7 or 14 days after administration of the bacteria. In some embodiments, the subject achieves at least 10% reduction in blood phenylalanine, e.g., as measured 7 or 14 days after administration of the bacteria. In some embodiments, the subject achieves at least 20% reduction in blood phenylalanine, e.g., as measured 7 or 14 days after administration of the bacteria. In some embodiments, the subject achieves at least 30% reduction in blood phenylalanine, e.g., as measured 7 or 14 days after administration of the bacteria. In some embodiments, the subject receives labeled phenylalanine, e.g., D5-phenylalanine, with the genetically engineered bacteria, and the labeled phenylalanine levels are decreased by at least 10%, at least 15%, at least 20%, at least 25%, at least 30%, at least 35%, at least 40%, at least 45%, or at least 50% as compared to baseline before administration. Labeled TCA may be detectable and increase proportionately with phenylalanine reduction.

Before, during, and after the administration of the pharmaceutical composition, phenylalanine levels in the subject may be measured in a biological sample, such as blood, serum, plasma, urine, peritoneal fluid, cerebrospinal fluid, fecal matter, intestinal mucosal scrapings, a sample collected from a tissue, and/or a sample collected from the contents of one or more of the following: the stomach, duodenum, jejunum, ileum, cecum, colon, rectum, and anal canal. In some embodiments, the methods may include administration of the compositions to reduce phenylalanine. In some embodiments, the methods may include administration of the compositions to reduce phenylalanine to undetectable levels in a subject. In some embodiments, the methods may include administration of the compositions to reduce phenylalanine concentrations to undetectable levels, or to less than about 1%, 2%, 5%, 10%, 20%, 25%, 30%, 40%, 50%, 60%, 70%, 75%, or 80% of the subject's phenylalanine levels prior to treatment. In some embodiments, the methods may include administration of the compositions to reduce phenylalanine levels below at least 600 μmol/L, at least 360 μmol/L, at least 180 μmol/L, at least 120 μmol/L or to levels between 360 μmol/L and 180 μmol/L or to levels between 180 μmol/L and 120 μmol/L.

In certain embodiments, the genetically engineered bacteria are E. coli Nissle. The genetically engineered bacteria may be destroyed, e.g., by defense factors in the gut or blood serum (Sonnenborn et al., 2009) or by activation of a kill switch, several hours or days after administration. Thus, the pharmaceutical composition comprising the genetically engineered bacteria may be re-administered at a therapeutically effective dose and frequency. In alternate embodiments, the genetically engineered bacteria are not destroyed within hours or days after administration and may propagate and colonize the gut.

The methods may comprise administration of the pharmaceutical composition alone or in combination with one or more additional therapeutic agents, such as an additional phe management regimen, which serves to lower blood plasma phe levels. In some embodiments, the pharmaceutical composition is administered in conjunction with a pterin cofactor, having a pteridine ring. Non-limiting examples include tetrahydrobiopterin and sepiapterin. In some embodiments, the pharmaceutical composition is administered in conjunction with the cofactor tetrahydrobiopterin (e.g., Kuvan/sapropterin), sepiapterin, large neutral amino acids (e.g., tyrosine, tryptophan), glycomacropeptides, a probiotic (e.g., VSL3), an enzyme (e.g., pegylated-PAL), and/or other agents used in the treatment of phenylketonuria (Al Hafid and Christodoulou, 2015). In some instances the patient may already be receiving the additional therapeutic agent or phe management regimen at baseline, prior to the initiation of administration of the genetically engineered bacteria.

A consideration in the selection of the one or more additional therapeutic agents is that the agent(s) should be compatible with the genetically engineered bacteria of the invention, e.g., the agent(s) must not interfere with or kill the bacteria. In some embodiments, the pharmaceutical composition is administered with food. In alternate embodiments, the pharmaceutical composition is administered before or after eating food. The pharmaceutical composition may be administered in combination with one or more dietary modifications, e.g., low-phenylalanine diet. The dosage of the pharmaceutical composition and the frequency of administration may be selected based on the severity of the symptoms and the progression of the disease. The appropriate therapeutically effective dose and/or frequency of administration can be selected by a treating clinician.

The methods also include kits comprising the pharmaceutical composition described herein. The kit can include one or more other elements including, but not limited to: instructions for use; other reagents, e.g., a label, an additional therapeutic agent; devices or materials for measuring phenylalanine levels, or levels of other molecules or metabolites associated with hyperphenylalaninemia, in a subject; devices or other materials for preparing the pharmaceutical composition for administration; and devices or other materials for administration to a subject. Instructions for use can include guidance for therapeutic application, such as suggested dosages and/or modes of administration, e.g., in a patient with hyperphenylalaninemia. The kit can further contain at least one additional therapeutic agent, and/or one or more additional genetically engineered bacterial strains of the invention, formulated as appropriate, in one or more separate pharmaceutical preparations.

In some embodiments, the kit is used for administration of the pharmaceutical composition to a subject. In some embodiments, the kit is used for administration of the pharmaceutical composition, alone or in combination with one or more additional therapeutic agents, to a subject. In some embodiments, the kit is used for measuring phenylalanine levels (e.g., blood phenylalanine levels) in a subject before, during, or after administration of the pharmaceutical composition to the subject. In certain embodiments, the kit is used for administration and/or re-administration of the pharmaceutical composition, alone or in combination with one or more additional therapeutic agents, when blood phenylalanine levels are increased or abnormally high, e.g., where levels are greater than 360 μmol/L, greater than 600 μmol/L or greater than μmol/L or ranger from at least 360 μmol/L to 600 μmol/L, at least 600 to 1200 μmol/L.

Phenylalanine may be measured by methods known in the art, e.g., blood sampling and mass spectrometry. Pyruvic acid and phenylpyruvate, the LAAD generated degradation products can be measured using mass spectrometry as described in the art and can be used as an additional readout of LAAD activity.

In some embodiments, the subject described herein is between 18 and 64 years of age. In some embodiments, the subject does not have an acute or chronic medical (including COVID-19 infection), surgical, psychiatric, or social condition or laboratory abnormality that may increase subject risk associated with study participation, compromise adherence to study procedures and requirements, or may confound interpretation of study safety or PD results and, in the judgment of the investigator, would make the subject inappropriate for enrollment. In some embodiments, the subject does not have a body mass index (BMI)<18.5 or ≥35 kg/m². In some embodiments, the subject does not have history of or current immunodeficiency disorder including human immunodeficiency virus (HIV) antibody positivity. In some embodiments, the subject does not have hepatitis B surface antigen positivity. In other embodiments, subjects with hepatitis B surface antibody positivity and hepatitis B core antibody positivity are not excluded, provided that the hepatitis B surface antigen is negative. In some embodiments, the subject does not have hepatitis C antibody positivity, unless a hepatitis C virus ribonucleic acid test is performed, and the result is negative. In some embodiments, the subject does not have a history of febrile illness, confirmed bacteremia, or other active infection deemed clinically significant by the investigator within 30 days prior to the anticipated first dose of the genetically engineered bacteria described herein. In some embodiments, the subject does not have a history of (within the past month) passage of 3 or more loose stools per day, where “loose stool” is defined as a Type 6 or Type 7 on the Bristol Stool Chart. In some embodiments, the subject does not have inflammatory irritable bowel disorder of any grade experienced within the previous 60 days. In some embodiments, the subject does not have an active or past history of GI bleeding within 60 days prior to the Screening Visit as confirmed by hospitalization-related event(s) or medical history of hematemesis or hematochezia. In some embodiments, the subject does not have intolerance of or allergic reaction to EcN, esomeprazole or any of the ingredients in the formulation to be administered. In other embodiments, the subject does not have intolerance of or allergic reaction to any of the ingredients in the formulation to be administered. In some embodiments, the subject does not have any condition (e.g., celiac disease, gastrectomy, bypass surgery, ileostomy), prescription medication, or over-the-counter product that may possibly affect absorption of medications or nutrients. In some embodiments, the subject is not currently taking or planning to take any type of systemic (e.g., oral or intravenous) antibiotic within 30 days prior to Day −1 through the final day of inpatient monitoring. In some embodiments, the subject does not have major surgery (an operation upon an organ within the cranium, chest, abdomen, or pelvic cavity) or inpatient hospital stay within the past 3 months prior to Screening. In some embodiments, the subject does not have planned surgery, hospitalizations, dental work, or interventional studies between Screening and last anticipated visit that might require antibiotics. In some embodiments, the subject is not taking or planning to take probiotic supplements (enriched foods excluded) within 30 days prior to Day −1 through the Safety Follow-up Period. In some embodiments, the subject does not have dependence on alcohol or drugs of abuse. In some embodiments, the subject does not have administration or ingestion of an investigational drug within 30 days or 5 half-lives, whichever is longer, prior to the Screening Visit, or current enrollment in an investigational study. In some embodiments, the subject has received a COVID-19 vaccine 7 days prior to the anticipated first dose of IMP or 7 days after the last dose of IMP. In some embodiments, the subject does not have administration or ingestion of a PPI within 30 days prior to Day −2. In some embodiments, the subject has screening laboratory parameters (e.g., chemistry panel, hematology, coagulation) and ECG inside of the normal limits based on standard ranges. In other embodiments, the subject has screening laboratory parameters defined as white blood cells 3.0-14.0×10⁹/L, platelets>100×10⁹/L, hemoglobin>10 g/dL, estimated glomerular filtration rate (eGFR) by the Chronic Kidney Disease Epidemiology Collaboration equation>60 mL/min/1.73 m², aspartate aminotransferase (AST)≤2×upper limit of normal (ULN), alanine aminotransferase (ALT)≤2×ULN, bilirubin<ULN, unless diagnosed with Gilbert's syndrome. In still other embodiments, the subject has screening laboratory parameters judged not to be clinically significant by the investigator. A single repeat evaluation of screening laboratory parameters is acceptable.

In some embodiments, the subject is 18 years of age or older. In some embodiments, the subject is younger than 18 years of age. In some embodiments, the subject is 12 years of age or older. In some embodiments, the subject has a diagnosis of classic PKU based on medical history as assessed by the investigator (e.g., Phe concentration of >1200 μmol/L at any time, low dietary Phe tolerance, or genetic diagnosis). In some embodiments, the subject has blood Phe≥600 μmol/L at Screening at current treatment regimen (diet and/or sapropterin and or sepiapterin at a stable dose). In some embodiments, the subject is on a stable diet including stable medical formula regimen (if used) for at least 1 month prior to screening. In some embodiments, the subject is available for and agrees to all study procedures, including urine and blood collection, adherence to diet control, follow-up visits, and ingestion compliance with the genetically engineered bacteria described herein. In some embodiments, the subject has screening laboratory evaluations (e.g., chemistry panel, complete blood count [CBC] with differential, urinalysis, creatinine clearance, CRP) within normal limits or judged to be not clinically significant by the investigator.

In some embodiments, the subject is not currently taking Palynziq® (pegvaliase-pqpz) within 1 month of screening. In some embodiments, the subject is currently taking Palynziq® (pegvaliase-pqpz). In some embodiments, the subject does not have inflammatory bowel disease of any grade or irritable bowel syndrome requiring pharmacologic therapy. In some embodiments, the subject has inflammatory bowel disease. In some embodiments, the subject does not have a history of or current immunodeficiency disorder. In some embodiments, the subject does not have intolerance of or allergic reaction to E. coli Nissle or any of the ingredients in the formulation to be administered. In some embodiments, the subject does not have any condition (e.g., celiac disease, gastrectomy, bypass surgery, ileostomy) or is not receiving prescription medication or an over-the-counter product that may possibly affect absorption of medications or nutrients. In some embodiments, the subject is not currently taking or planning to take any type of systemic (e.g., oral or intravenous) antibiotic within 28 days prior to the first dose of IMP through final safety assessment, including planned surgery, hospitalizations, dental procedures, or interventional studies that are expected to require antibiotics. In some embodiments, the subject does not have, within the 3 months prior to anticipated first dose, major surgery (an operation upon an organ within the cranium, chest, abdomen, or pelvic cavity) or inpatient hospital stay. In some embodiments, the subject does not have dependence on alcohol or drugs of abuse. In some embodiments, the subject does not have administration or ingestion of an investigational drug within 30 days or 5 half-lives, whichever is longer, prior to the Screening Visit, or current enrollment in an investigational study. In some embodiments, the subject does not have acute or chronic medical, surgical, psychiatric, or social condition or laboratory abnormality that may increase patient risk associated with study participation, compromise adherence to study procedures and requirements, or may confound interpretation of study safety or PD results and, in the judgment of the investigator, would make the patient inappropriate for enrollment.

In some embodiments, the subject takes a suitable dose of a proton pump inhibitor (PPI), e.g., esomeprazole 40 mg QD, before the same meal, e.g., 60 to 90 minutes before the meal, from about Day −7 through Day 15 (or last dose of the genetically engineered bacteria described herein). In these embodiments, the PPI is taken at the same time, even if no meal is consumed. If patients are already on a PPI regimen, they may continue on that and not switch to esomeprazole. In case of intolerance to esomeprazole, another PPI may be used.

In some embodiments, the bacteria described herein are administered to a subject on a phe-restricted diet.

In some embodiments, the bacteria described herein may be administered in conjunction with a second therapy, e.g., a second phenylalanine reduction therapy. In some embodiments, the bacteria and the second therapy are administered concurrently. In some embodiments, the bacteria and the second therapy are administered sequentially, i.e., the second therapy is administered before or after the bacteria. In some embodiments, the second therapy is an oral therapy. In some embodiments, the second therapy is administered parenterally.

In some embodiments, the second therapy is sapropterin dihydrochloride (Kuvan®). Sapropterin dihydrochloride (Kuvan®) is administered to patients with hyperphenylalaninemia (HPA) due to tetrahydrobiopterin-(BH4-) responsive Phenylketonuria to reduce blood phenylalanine (Phe) levels, and is generally used in conjunction with a Phe-restricted diet.

Accordingly, in some embodiments, the methods of treatment comprising administering the bacterium as described herein may further include administering a second therapy, e.g., a phenylalanine lowering therapy, e.g., a Sapropterin dihydrochloride therapy. In some embodiments, the therapy comprises administering 10 to 20 mg/kg Sapropterin dihydrochloride once daily. In some embodiments, the therapy comprises administering 20 mg/kg Sapropterin dihydrochloride once daily.

### Genetically Engineered Bacteria for Reducing Hyperphenylalaninemia

The genetically engineered bacteria disclosed herein are capable of reducing excess phenylalanine. In some embodiments, the genetically engineered bacteria are non-pathogenic bacteria. In some embodiments, the genetically engineered bacteria are commensal bacteria. In some embodiments, the genetically engineered bacteria are probiotic bacteria. In some embodiments, the genetically engineered bacteria are naturally pathogenic bacteria that are modified or mutated to reduce or eliminate pathogenicity. In some embodiments, non-pathogenic bacteria are Gram-negative bacteria. In some embodiments, non-pathogenic bacteria are Gram-positive bacteria. Exemplary bacteria include, but are not limited to, Bacillus, Bacteroides, Bifidobacterium, Brevibacteria, Clostridium, Enterococcus, Escherichia coli, Lactobacillus, Lactococcus, Saccharomyces, and Staphylococcus, e.g., Bacillus coagulans, Bacillus subtilis, Bacteroides fragilis, Bacteroides subtilis, Bacteroides thetaiotaomicron, Bifidobacterium bifidum, Bifidobacterium infantis, Bifidobacterium lactis, Bifidobacterium longum, Clostridium butyricum, Enterococcus faecium, Lactobacillus acidophilus, Lactobacillus bulgaricus, Lactobacillus casei, Lactobacillus johnsonii, Lactobacillus paracasei, Lactobacillus plantarum, Lactobacillus reuteri, Lactobacillus rhamnosus, Lactococcus lactis, and Saccharomyces boulardii. In certain embodiments, the genetically engineered bacteria are selected from the group consisting of Bacteroides fragilis, Bacteroides thetaiotaomicron, Bacteroides subtilis, Bifidobacterium bifidum, Bifidobacterium infantis, Bifidobacterium lactis, Clostridium butyricum, Escherichia coli Nissle, Lactobacillus acidophilus, Lactobacillus plantarum, Lactobacillus reuteri, and Lactococcus lactis.

In some embodiments, the genetically engineered bacteria are Escherichia coli strain Nissle 1917 (E. coli Nissle), a Gram-negative bacterium of the Enterobacteriaceae family that has evolved into one of the best characterized probiotics (Ukena et al., 2007). The strain is characterized by its complete harmlessness (Schultz, 2008), and has GRAS (generally recognized as safe) status (Reister et al., 2014, emphasis added). Genomic sequencing confirmed that E. coli Nissle lacks prominent virulence factors (e.g., E. coli α-hemolysin, P-fimbrial adhesins) (Schultz, 2008). In addition, it has been shown that E. coli Nissle does not carry pathogenic adhesion factors, does not produce any enterotoxins or cytotoxins, is not invasive, and is not uropathogenic (Sonnenborn et al., 2009). As early as in 1917, E. coli Nissle was packaged into medicinal capsules, called Mutaflor, for therapeutic use. It is commonly accepted that E. coli Nissle's therapeutic efficacy and safety have convincingly been proven (Ukena et al., 2007).

One of ordinary skill in the art would appreciate that the genetic modifications disclosed herein may be adapted for other species, strains, and subtypes of bacteria. Furthermore, genes from one or more different species can be introduced into one another, e.g., the PAL gene from Rhodosporidium toruloides can be expressed in Escherichia coli (Sarkissian et al., 1999), and it is known that prokaryotic and eukaryotic phenylalanine ammonia lyases share sequence homology (Xiang and Moore, 2005).

Unmodified E. coli Nissle and the genetically engineered bacteria disclosed herein may be destroyed, e.g., by defense factors in the gut or blood serum (Sonnenborn et al., 2009) or by activation of a kill switch, several hours or days after administration. Thus, the genetically engineered bacteria may require continued administration. In some embodiments, the residence time is calculated for a human subject. Residence time in vivo may be calculated for the genetically engineered bacteria of the invention.

In some embodiments, the genetically engineered bacteria comprise a gene encoding a PME. In some embodiments, the gene encoding the PME is operably linked to a directly or indirectly inducible promoter. In some embodiments, the PME is operably linked to a constitutive promoter. In some embodiments, the bacteria comprise a non-native gene encoding a PME. In some embodiments, the bacteria comprise additional copies of a native gene encoding a PME. In some embodiments, the promoter is not associated with the gene encoding the PME in nature. In some embodiments, the genetically engineered bacteria comprise a gene encoding PAL. In some embodiments, the PAL gene is operably linked to a directly or indirectly inducible promoter. In some embodiments, the PAL gene is operably linked to a constitutive promoter. In some embodiments, the bacteria comprise a non-native PAL gene. In some embodiments, the bacteria comprise additional copies of a native PAL gene. In some embodiments, the promoter is not associated with the PAL gene in nature. In some embodiments, the genetically engineered bacteria comprise a gene encoding a LAAD. In some embodiments, the gene encoding the LAAD is operably linked to a directly or indirectly inducible promoter. In some embodiments, the gene encoding LAAD is operably linked to a constitutive promoter. In some embodiments, the bacteria comprise a non-native gene encoding a LAAD. In some embodiments, the bacteria comprise additional copies of a native gene encoding a LAAD. In some embodiments, the promoter is not associated with the gene encoding the LAAD in nature. In some embodiments, the genetically engineered bacteria comprise a gene encoding PAH, wherein the PAH gene is operably linked to a directly or indirectly inducible promoter. In some embodiments, the bacteria comprise a non-native PAH gene. In some embodiments, the bacteria comprise additional copies of a native PAH gene. In some embodiments, the promoter is not associated with the PAH gene in nature.

The genetically engineered bacteria further comprise a gene encoding a phenylalanine transporter (PheP). In certain embodiments, the bacteria comprise additional copies of a native gene encoding a phenylalanine transporter, wherein the phenylalanine transporter gene is operably linked to a promoter, e.g., an inducible promoter. In alternate embodiments, the bacteria comprise a gene encoding a non-native phenylalanine transporter, wherein the phenylalanine transporter gene is operably linked to a promoter, e.g., an inducible promoter. Both embodiments are encompassed by the term “non-native” phenylalanine transporter. In some embodiments, the promoter is not associated with the pheP gene in nature. In some embodiments, the same promoter controls expression of PheP and PAL or PAH.

PheP is a membrane transport protein that is capable of transporting phenylalanine into bacterial cells (see, e.g., Pi et al., 1991). In some embodiments, the native pheP gene in the genetically modified bacteria is not modified. In some embodiments, the genetically engineered bacteria comprise multiple copies of the native pheP gene. In some embodiments, the genetically engineered bacteria comprise multiple copies of a non-native pheP gene. In some embodiments, the genetically engineered bacteria comprise a pheP gene that is controlled by its native promoter, an inducible promoter, a promoter that is stronger than the native promoter, e.g., the GlnRS promoter or the P(Bla) promoter, or a constitutive promoter. In some embodiments, expression of the pheP gene is controlled by a different promoter than the promoter that controls expression of the gene encoding the phenylalanine-metabolizing enzyme and/or the transcriptional regulator. In some embodiments, expression of the pheP gene is controlled by the same promoter that controls expression of the phenylalanine-metabolizing enzyme and/or the transcriptional regulator. In some embodiments, the pheP gene and the phenylalanine-metabolizing enzyme and/or the transcriptional regulator are divergently transcribed from a promoter region. In some embodiments, expression of each of the genes encoding PheP, the phenylalanine-metabolizing enzyme, and the transcriptional regulator is controlled by a different promoter. In some embodiments, expression of the genes encoding PheP, the phenylalanine-metabolizing enzyme, and the transcriptional regulator is controlled by the same promoter.

In some embodiments, the native pheP gene in the genetically modified bacteria is not modified, and one or more additional copies of the native pheP gene are inserted into the genome under the control of the same inducible promoter that controls expression of PAL, e.g., an FNR promoter, an IPTG-inducible promoter, or a different inducible promoter than the one that controls expression of PAL, or a constitutive promoter. In alternate embodiments, the native pheP gene is not modified, and a copy of a non-native pheP gene from a different bacterial species is inserted into the genome under the control of the same inducible promoter that controls expression of PAL, e.g., an FNR promoter, an IPTG-inducible promoter, or a different inducible promoter than the one that controls expression of PAL or a constitutive promoter.

In some embodiments, the native pheP gene in the genetically modified bacteria is not modified, and one or more additional copies of the native pheP gene are present in the bacteria on a plasmid and under the control of the same inducible promoter that controls expression of PAL, e.g., an FNR promoter, an IPTG-inducible promoter, or a different inducible promoter than the one that controls expression of the PME, or a constitutive promoter. In alternate embodiments, the native pheP gene is not modified, and a copy of a non-native pheP gene from a different bacterial species is present in the bacteria on a plasmid and under the control of the same inducible promoter that controls expression of PAL, e.g., an FNR promoter, an IPTG-inducible promoter, or a different inducible promoter than the one that controls expression of PAL or a constitutive promoter.

In some embodiments, the native pheP gene is mutagenized, mutants exhibiting increased phenylalanine transport are selected, and the mutagenized pheP gene is isolated and inserted into the genetically engineered bacteria (see, e.g., Pi et al., 1996; Pi et al., 1998). The phenylalanine transporter modifications described herein may be present on a plasmid or chromosome.

In some embodiments, the genetically engineered bacterium is E. coli Nissle, and the native pheP gene in E. coli Nissle is not modified; one or more additional copies of the native E. coli Nissle pheP genes are inserted into the E. coli Nissle genome under the control of the same inducible promoter that controls expression of PAL, e.g., an FNR promoter or an IPTG-inducible promoter, or a different inducible promoter than the one that controls expression of PAL or a constitutive promoter. In an alternate embodiment, the native pheP gene in E. coli Nissle is not modified, and a copy of a non-native pheP gene from a different bacterium is inserted into the E. coli Nissle genome under the control of the same inducible promoter that controls expression of PAL, e.g., an FNR promoter or an IPTG-inducible promoter, or a different inducible promoter than the one that controls expression of PAL or a constitutive promoter. In some embodiments, the genetically engineered bacterium is E. coli Nissle, and the native pheP gene in E. coli Nissle is not modified; one or more additional copies the native E. coli Nissle pheP genes are present in the bacterium on a plasmid and under the control of the same inducible promoter that controls expression of PAL, e.g., an FNR promoter or an IPTG-inducible promoter, or a different inducible promoter than the one that controls expression of PAL, or a constitutive promoter. In an alternate embodiment, the native pheP gene in E. coli Nissle is not modified, and a copy of a non-native pheP gene from a different bacterium, are present in the bacterium on a plasmid and under the control of the same inducible promoter that controls expression of PAL, e.g., an FNR promoter or an IPTG inducible promoter, or a different inducible promoter than the one that controls expression of PAL or a constitutive promoter.

It has been reported that Escherichia coli has five distinct transport systems (AroP, Mtr, PheP, TnaB, and TyrP) for the accumulation of aromatic amino acids. A general amino acid permease, encoded by the aroP gene, transports three aromatic amino acids, including phenylalanine, with high affinity, and is thought, together with PheP, responsible for the lion share of phenylalanine import. Additionally, a low level of accumulation of phenylalanine was observed in an aromatic amino acid transporter-deficient E. coli strain (ΔaroP ΔpheP Δmtr Δtna ΔtyrP), and was traced to the activity of the LIV-I/LS system, which is a branched-chain amino acid transporter consisting of two periplasmic binding proteins, the LIV-binding protein (LIV-I system) and LS-binding protein (LS system), and membrane components, LivHMGF (Koyanagi et al., and references therein; Identification of the LIV-I/LS System as the Third Phenylalanine Transporter in Escherichia coli K-12).

In some embodiments, the genetically engineered bacteria comprise an aroP gene. In some embodiments, the genetically engineered bacterium is E. coli Nissle, and the native aroP gene in E. coli Nissle is not modified; one or more additional copies of the native E. coli Nissle aroP genes are present in the bacterium on a plasmid or in the chromosome and under the control of the same inducible promoter that controls expression of the PME, e.g., an FNR promoter, an araBAD promoter, an IPTG-inducible promoter, a different inducible promoter than the one that controls expression of the PME, or a constitutive promoter. In an alternate embodiment, the native aroP gene in E. coli Nissle is not modified, and a copy of a non-native aroP gene from a different bacterium, are present in the bacterium on a plasmid or in the chromosome and under the control of the same inducible promoter that controls expression of the PME, e.g., an FNR promoter, an AraBAD promoter, or an IPTG-inducible promoter, or a different inducible promoter than the one that controls expression of the PME, or a constitutive promoter.

In other embodiments, the genetically engineered bacteria comprise AroP and PheP, under the control of the same or different inducible or constitutive promoters.

In some embodiments, the pheP gene is expressed on a chromosome. In some embodiments, expression from the chromosome may be useful for increasing stability of expression of pheP. In some embodiments, the pheP gene is integrated into the bacterial chromosome at one or more integration sites in the genetically engineered bacteria. In some embodiments, the pheP gene is inserted into the bacterial genome at one or more of the following insertion sites in E. coli Nissle: malE/K, insB/I, araC/BAD, lacZ, agal/rsml, thyA, and malP/T. The insertion site may be anywhere in the genome, e.g., in a gene required for survival and/or growth, such as thyA (to create an auxotroph); in an active area of the genome, such as near the site of genome replication; and/or in between divergent promoters in order to reduce the risk of unintended transcription, such as between AraB and AraC of the arabinose operon.

In some embodiments, the genetically engineered bacterium comprises multiple mechanisms of action and/or one or more auxotrophies. In certain embodiments, the bacteria are genetically engineered to comprise five copies of PAL under the control of an oxygen level-dependent promoter (e.g., P_{fnrS}-PAL3) inserted at different integration sites on the chromosome (e.g., mal/K, yicS/nepI, malP/T, agaI/rsmI, and cea), and one copy of a phenylalanine transporter gene under the control of an oxygen level-dependent promoter (e.g., P_{fnrS}-pheP) inserted at a different integration site on the chromosome (e.g., lacZ). In a more specific aspect, the bacteria are genetically engineered to further include a kanamycin resistance gene, and a thyA auxotrophy, in which the thyA gene is deleted and/or replaced with an unrelated gene.

Phenylalanine ammonia lyase (PAL; EC 4.3.1.24) is an enzyme that catalyzes a reaction converting L-phenylalanine to ammonia and trans-cinnamic acid. Phenylalanine ammonia lyase is specific for L-Phe, and to a lesser extent, L-Tyrosine. The reaction catalyzed by PAL is the spontaneous, non-oxidative deamination of L-phenylalanine to yield trans-cinnamic acid and ammonia. Unlike the mammalian enzyme (PAH), PAL is a monomer and requires no cofactors (MacDonald et al., Biochem Cell Biol 2007; 85:273-82. A modern view of phenylalanine ammonia lyase). In micro-organisms, it has a catabolic role, allowing them to utilize L-phenylalanine (L-Phe) as a sole source of carbon and nitrogen. In one embodiment, the genetically engineered bacteria comprise a PAL gene. PAL is capable of converting phenylalanine to non-toxic levels of transcinnamic acid and ammonia. Trans-cinnamic acid (TCA) can further be converted to TCA metabolites benzoic and hippuric acids (Sarkissian et al., J Mass Spectrom. 2007 June; 42(6):811-7; Quantitation of phenylalanine and its trans-cinnamic, benzoic and hippuric acid metabolites in biological fluids in a single GC-MS analysis). PAL enzyme activity does not require THB cofactor activity.

In some embodiments, PAL is encoded by a PAL gene derived from a bacterial species, including but not limited to, Achromobacter xylosoxidans, Pseudomonas aeruginosa, Photorhabdus luminescens, Anabaena variabilis, and Agrobacterium tumefaciens. In some embodiments, the bacterial species is Photorhabdus luminescens. In some embodiments, the bacterial species is Anabaena variabilis. In some embodiments, PAL is encoded by a PAL gene derived from a eukaryotic species, e.g., a yeast species, a plant species. Multiple distinct PAL proteins are known in the art. The genetically engineered bacteria convert more phenylalanine when the PAL gene is expressed than unmodified bacteria of the same bacterial subtype under the same conditions. Thus, the genetically engineered bacteria comprising PAL may be used to metabolize phenylalanine in the body into non-toxic molecules in order to treat conditions associated with hyperphenylalaninemia, including PKU. In some embodiments, the genetically engineered bacteria express Anabaena variabilis PAL (“PAL1”). In some embodiments, the genetically engineered bacteria express Photorhabdus luminescens PAL (“PAL3”). Non-limiting examples of PAL sequences of interest are provided herein and in the art.

LAAD catalyzes the stereospecific oxidative, i.e., oxygen consuming, deamination of L-amino acids to α-keto acids along with the production of ammonia and hydrogen peroxide via an amino acid intermediate. L-AADs are found in snake venoms, and in many bacteria (Bifulco et al. 2013), specifically in the cytomembranes of the Proteus, Providencia, and Morganella bacteria. L-AADs (EC 1.4.3.2) are flavoenzymes with a dimeric structure. Each subunit contains a non-covalently-bound flavin adenine dinucleotide (FAD) cofactor) and do not require any external cofactors. Proteus mirabilis contains two types of L-AADs (Duerre and Chakrabarty 1975). One has broad substrate specificity and catalyzes the oxidation of aliphatic and aromatic L-amino acids to keto acids, typically L-phenylalanine (GenBank: U35383.1) (Baek et al., Journal of Basic Microbiology 2011, 51, 129-135; “Expression and characterization of a second L-amino acid deaminase isolated from Proteus mirabilis in Escherichia coli”). The other type acts mainly on basic L-amino acids (GenBank: EU669819.1). LAADs from bacterial, fungal, and plant sources appear to be involved in the utilization of L-amino acids (i.e., ammonia produced by the enzymatic activity) as a nitrogen source. Most eukaryotic and prokaryotic L-amino acid deaminases are extracellularly secreted, with the exception of from Proteus species LAADs, which are membrane-bound. In Proteus mirabilis, L-AADs have been reported to be located in the plasma membrane, facing outward into the periplasmic space, in which the enzymatic activity resides (Pelmont J et al., (1972) “L-amino acid oxidases of Proteus mirabilis: general properties” Biochimie 54: 1359-1374).

In one embodiment, the genetically engineered bacteria comprise a LAAD gene. LAAD is capable of converting phenylalanine to non-toxic levels of phenylpyruvate, which can also further be degraded, e.g., by liver enzymes, to phenyllactate. Phenylpyruvate cannot cross the blood brain barrier, which allows LAAD to reduce the levels of phenylalanine in the brain without allowing the accumulation of another potentially toxic metabolite. In some embodiments, LAAD is encoded by a LAAD gene derived from a bacterial species, including but not limited to, Proteus, Providencia, and Morganella bacteria. In some embodiments, the bacterial species is Proteus mirabilis. In some embodiments, the bacterial species is Proteus vulgaris. In some embodiments, the genetically engineered bacteria express Proteus mirabilis LAAD enzyme GenBank: U35383.1. Non-limiting examples of LAAD sequences are provided herein and known in the art. In some embodiments, the LAAD enzyme is derived from snake venom. According to the invention, genetically engineered bacteria convert more phenylalanine when the LAAD gene is expressed than unmodified bacteria of the same bacterial subtype under the same conditions. Thus, the genetically engineered bacteria comprising LAAD may be used to metabolize phenylalanine in the body into non-toxic molecules in order to treat conditions associated with hyperphenylalaninemia, including PKU.

The PME, e.g., PAL, LAAD, or PAH, gene may be present on a plasmid or chromosome in the genetically engineered bacteria. In some embodiments, the PME gene is expressed under the control of a constitutive promoter. In some embodiments, the PME gene is expressed under the control of a promoter that is directly or indirectly induced by exogenous environmental conditions, as described herein. In some embodiments, the PME gene is expressed under the control of a promoter that is directly or indirectly induced by exogenous environmental conditions, such as in the presence of molecules or metabolites specific to the gut of a mammal. In one embodiment, the PME gene is expressed under the control of a promoter that is directly or indirectly induced by low-oxygen, microaerobic, or anaerobic conditions, wherein expression of the PME gene, e.g., the PAL gene, is activated under low-oxygen or anaerobic environments, such as the environment of the mammalian gut.

In some embodiments, the promoter that is operably linked to PAL, PAH, and/or pheP is an inducible promoter. In some embodiments, the promoter is induced by exogenous environmental conditions specific to the gut of a mammal. In some embodiments, the promoter is induced by exogenous environmental conditions specific to the small intestine of a mammal. In some embodiments, the promoter is induced by low-oxygen or anaerobic conditions such as the environment of the mammalian gut. In some embodiments, the promoter is induced by the presence of molecules or metabolites that are specific to the gut of a mammal, e.g., propionate. In some embodiments, the promoter is induced by exposure to tetracycline. In some embodiments, the promoter is induced a molecule that is co-administered with the genetically engineered bacteria of the invention.

In some embodiments, the genetically engineered bacteria encode a PAL gene which is induced by low-oxygen or anaerobic conditions, such as the mammalian gut. In some embodiments, the genetically engineered bacteria encode a PAL gene which is induced by oxygenated, low oxygen, or microaerobic conditions, such as conditions found in the proximal intestine, including but not limited to the stomach, duodenum, and ileum. In some embodiments, the genetically engineered bacteria encode a PAL gene which is induced by an environmental factor that is naturally present in a mammalian gut. In some embodiments, the genetically engineered bacteria encode a PAL gene which is induced by an environmental factor that is not naturally present in a mammalian gut, e.g., arabinose. In some embodiments, the genetically engineered bacteria encode a PAL gene which is induced by an environmental factor that is naturally present in a mammalian gut under inflammatory conditions.

Bacteria have evolved transcription factors that are capable of sensing oxygen levels. Different signaling pathways may be triggered by different oxygen levels and occur with different kinetics. An oxygen level-dependent promoter is a nucleic acid sequence to which one or more oxygen level-sensing transcription factors is capable of binding, wherein the binding and/or activation of the corresponding transcription factor activates downstream gene expression. In one embodiment, the PME gene is expressed under the control of an oxygen level-dependent promoter. In a more specific aspect, the PAL gene is under the control of an oxygen level-dependent promoter that is activated under low-oxygen or anaerobic environments, such as the environment of the mammalian gut.

In certain embodiments, the genetically engineered bacteria comprise a PME, e.g., PAL, expressed under the control of the fumarate and nitrate reductase regulator (FNR) promoter. In E. coli, FNR is a major transcriptional activator that controls the switch from aerobic to anaerobic metabolism (Unden et al., 1997). In the anaerobic state, FNR dimerizes into an active DNA binding protein that activates hundreds of genes responsible for adapting to anaerobic growth. In the aerobic state, FNR is prevented from dimerizing by oxygen and is inactive. In some embodiments, multiple distinct FNR nucleic acid sequences are inserted in the genetically engineered bacteria. In alternate embodiments, the genetically engineered bacteria comprise a PME, e.g., PAL, expressed under the control of an alternate oxygen level-dependent promoter, e.g., an ANR promoter (Ray et al., 1997), a DNR promoter (Trunk et al., 2010). In some embodiments, phenylalanine metabolism is particularly activated in a low-oxygen or anaerobic environment, such as in the gut.

In P. aeruginosa, the anaerobic regulation of arginine deiminase and nitrate reduction (ANR) transcriptional regulator is “required for the expression of physiological functions which are inducible under oxygen-limiting or anaerobic conditions” (Winteler et al., 1996; Sawers 1991). P. aeruginosa ANR is homologous with E. coli FNR, and “the consensus FNR site (TTGAT - - - ATCAA) was recognized efficiently by ANR and FNR” (Winteler et al., 1996). Like FNR, in the anaerobic state, ANR activates numerous genes responsible for adapting to anaerobic growth. In the aerobic state, ANR is inactive. Pseudomonas fluorescens, Pseudomonas putida, Pseudomonas syringae, and Pseudomonas mendocina all have functional analogs of ANR (Zimmermann et al., 1991). Promoters that are regulated by ANR are known in the art, e.g., the promoter of the arcDABC operon (see, e.g., Hasegawa et al., 1998).

The FNR family also includes the dissimilatory nitrate respiration regulator (DNR) (Arai et al., 1995), a transcriptional regulator which is required in conjunction with ANR for “anaerobic nitrate respiration of Pseudomonas aeruginosa” (Hasegawa et al., 1998). For certain genes, the FNR-binding motifs “are probably recognized only by DNR” (Hasegawa et al., 1998). Any suitable transcriptional regulator that is controlled by exogenous environmental conditions and corresponding regulatory region may be used. Non-limiting examples include ArcA/B, ResD/E, NreA/B/C, and AirSR, and others are known in the art.

FNR promoter sequences are known in the art, and any suitable FNR promoter sequence(s) may be used in the genetically engineered bacteria of the invention. Any suitable FNR promoter(s) may be combined with any suitable PAL. Non-limiting FNR promoter sequences are provided in Table 4, and non-limiting PAL sequences are also provided herein.

IPTG is an allolactose mimic known in the art and used to induce transcription of genes having lac repressor operons within their promoter regions. In bacteria, the transcriptional regulator, LacI represses the expression of genes encoding proteins related to lactose metabolism in the absence of lactose. Once lactose is available, however, it is converted into allolactose, which is capable of binding LacI and thereby allosterically inhibiting the ability of LacI to bind DNA at the lac operator and, in doing so, allowing expression of downstream genes. In certain embodiments, the genetically engineered bacteria comprise a PME, e.g., PAL, expressed under the control of an IPTG-inducible promoter, e.g., Ptac. In certain embodiments, the genetically engineered bacteria comprise a PAL, PAH, LAAD, and/or pheP operably linked to an IPTG-inducible promoter. The IPTG-inducible promoter is a nucleic acid sequence to which an allolactose/IPTG level-sensing transcription factor, e.g., the lac repressor LacI, is capable of binding. In some embodiments, binding of the transcription factor to the nucleic acid sequence, e.g., a promoter or promoter region comprising a lac operon, represses downstream gene expression in the absence of IPTG. IPTG-inducible promoter sequences are known in the art, and any suitable IPTG-inducible promoter sequence(s) may be used in the genetically engineered bacteria of the invention. Any suitable IPTG-inducible promoter may be combined with any suitable PAL PAH, LAAD, and/or pheP. Non-limiting IPTG-inducible promoter sequences are provided in Table 5, and non-limiting PAL PAH, LAAD, and pheP sequences are also provided herein.

In some embodiments, the bacterium comprises a nucleic acid sequence that is at least 80%, at least 85%, at least 90%, at least 91%, at least 92%, at least 93%, at least 94%, at least 95%, at least 96%, at least 97%, at least 98%, at least 99%, or at least 100% identical to a promoter sequence in Table 5 or a functional fragment thereof. In some embodiments, the bacterium comprises a nucleic acid sequence having at least 90%, at least 91%, at least 92%, at least 93%, at least 94%, at least 95%, at least 96%, at least 97%, at least 98%, or at least 99% identity to, comprises, or consists of SEQ ID NO: 383. In some embodiments, the bacterium comprises a nucleic acid sequence having at least 90%, at least 91%, at least 92%, at least 93%, at least 94%, at least 95%, at least 96%, at least 97%, at least 98%, or at least 99% identity to, comprises, or consists of SEQ ID NO: 381. In some embodiments, the bacterium further comprises a gene sequence encoding a regulator (e.g., LacI repressor), which represses the activity of the IPTG-inducible promoter in the absence of the inducer. In some embodiments, the bacterium comprises a gene sequence encoding a repressor comprising a sequence having at least 90%, at least 91%, at least 92%, at least 93%, at least 94%, at least 95%, at least 96%, at least 97%, at least 98%, or at least 99% identity to, comprises, or consists of SEQ ID NO: 16. In some embodiments, the bacterium comprises a gene sequence encoding a repressor comprises a sequence having at least 90%, at least 91%, at least 92%, at least 93%, at least 94%, at least 95%, at least 96%, at least 97%, at least 98%, or at least 99% identity to, comprises, or consists of SEQ ID NO: 15. In some embodiments, the bacterium comprises a gene sequence encoding a repressor comprises a sequence having at least 90%, at least 91%, at least 92%, at least 93%, at least 94%, at least 95%, at least 96%, at least 97%, at least 98%, or at least 99% identity to, comprises, or consists of SEQ ID NO: 379. In some embodiments, the bacterium comprises a gene sequence encoding a repressor comprises a sequence having at least 90%, at least 91%, at least 92%, at least 93%, at least 94%, at least 95%, at least 96%, at least 97%, at least 98%, or at least 99% identity to, comprises, or consists of SEQ ID NO: 380. In some embodiments, the bacterium comprises a gene sequence encoding a repressor comprises a sequence having at least 90%, at least 91%, at least 92%, at least 93%, at least 94%, at least 95%, at least 96%, at least 97%, at least 98%, or at least 99% identity to, comprises, or consists of SEQ ID NO: 17. In some embodiments, the bacterium comprises a gene sequence encoding a repressor comprises a sequence having at least 90%, at least 91%, at least 92%, at least 93%, at least 94%, at least 95%, at least 96%, at least 97%, at least 98%, or at least 99% identity to, comprises, or consists of SEQ ID NO: 384. In these embodiments, the bacterium may additionally contain SEQ ID NO: 382, 332, or 333, or a sequence having at least 90%, at least 91%, at least 92%, at least 93%, at least 94%, at least 95%, at least 96%, at least 97%, at least 98%, or at least 99% identity to SEQ ID NO: 382, 332, or 333.

In some embodiments, the bacterium comprises endogenous gene(s) encoding the IPTG sensing transcriptional regulator, LacI. In some embodiments, the lacI gene is heterologous or non-native. In some embodiments, the gene encoding the IPTG level-sensing transcriptional regulator, e.g., LacI, is present on a plasmid. In some embodiments, the gene encoding the IPTG level-sensing transcriptional regulator, e.g., LacI, and the gene encoding the PME or phenylalanine transporter are present on different plasmids. In some embodiments, the gene encoding the IPTG level-sensing transcriptional regulator, e.g., LacI, and the gene encoding the PME or phenylalanine transporter are present on the same plasmid. In some embodiments, the gene encoding the IPTG level-sensing transcriptional regulator, e.g., LacI, is present on a chromosome. In some embodiments, the gene encoding the IPTG level-sensing transcriptional regulator, e.g., LacI, and the gene encoding the PME or phenylalanine transporter are present on different chromosomes. In some embodiments, the gene encoding the IPTG level-sensing transcriptional regulator, e.g., LacI, and the gene encoding the PME or phenylalanine transporter are present on the same chromosome, either at the same or a different insertion site. In some embodiments, expression of the transcriptional regulator is controlled by a different promoter than the promoter that controls expression of the gene encoding the PME or phenylalanine transporter, e.g., a constitutive promoter. In some embodiments, the transcriptional regulator and the phenylalanine decarboxylase or phenylalanine transporter are divergently transcribed from a promoter region.

In some embodiments, the bacterium disclosed herein comprises a nucleotide sequence that encodes a PAL sequence that is at least 80%, at least 85%, at least 90%, at least 91%, at least 92%, at least 93%, at least 94%, at least 95%, at least 96%, at least 97%, at least 98%, at least 99%, or 100% identical to a PAL amino acid sequence in Table 6 or a functional fragment thereof. In some embodiments, the bacterium further comprises a nucleic acid sequence that is at least 80%, at least 85%, at least 90%, at least 91%, at least 92%, at least 93%, at least 94%, at least 95%, at least 96%, at least 97%, at least 98%, at least 99%, or 100% identical to a promoter sequence in Table 4 or a functional fragment thereof.

In some embodiments, the bacterium disclosed herein comprises a nucleotide sequence that encodes a PAL sequence that is at least 80%, at least 85%, at least 90%, at least 91%, at least 92%, at least 93%, at least 94%, at least 95%, at least 96%, at least 97%, at least 98%, at least 99%, or 100% identical to a PAL amino acid sequence in Table 6 or a functional fragment thereof, wherein the PAL sequence is operably linked to a nucleic acid sequence that is at least 80%, at least 85%, at least 90%, at least 91%, at least 92%, at least 93%, at least 94%, at least 95%, at least 96%, at least 97%, at least 98%, at least 99%, or 100% identical to a promoter sequence in Table 4 or a functional fragment thereof. In some embodiments, the bacterium further comprises a nucleotide sequence that encodes a PAL sequence that is at least 80%, at least 85%, at least 90%, at least 91%, at least 92%, at least 93%, at least 94%, at least 95%, at least 96%, at least 97%, at least 98%, at least 99%, or 100% identical to a PAL amino acid sequence in Table 6 or a functional fragment thereof, wherein the PAL sequence is operably linked to a nucleic acid sequence that is at least 80%, at least 85%, at least 90%, at least 91%, 92%, at least 93%, at least 94%, at least 95%, at least 96%, at least 97%, at least 98%, at least 99%, or 100% identical to a promoter sequence in Table 5 or a functional fragment thereof.

In some embodiments, the bacterium disclosed herein comprises a nucleotide sequence that encodes a PAL sequence that is at least 80%, at least 85%, at least 90%, at least 91%, at least 92%, at least 93%, at least 94%, at least 95%, at least 96%, at least 97%, at least 98%, at least 99%, or 100% identical to a PAL amino acid sequence in Table 6 or a functional fragment thereof, wherein the PAL sequence is operably linked to a nucleic acid sequence that is at least 80%, at least 85%, at least 90%, at least 91%, at least 92%, at least 93%, at least 94%, at least 95%, at least 96%, at least 97%, at least 98%, at least 99%, or 100% identical to a promoter sequence in Table 5 or a functional fragment thereof.

In some embodiments, the bacterium further comprises a nucleotide sequence that encodes a PheP sequence that is at least 80%, at least 85%, at least 90%, at least 91%, at least 92%, at least 93%, at least 94%, at least 95%, at least 96%, at least 97%, at least 98%, at least 99%, or 100% identical to a PheP amino acid sequence encoded by the PheP nucleotide sequence within SEQ ID NO: 506 or a functional fragment thereof, wherein the PheP sequence is operably linked to a nucleic acid sequence that is at least 80%, at least 85%, at least 90%, at least 91%, at least 92%, at least 93%, at least 94%, at least 95%, at least 96%, at least 97%, at least 98%, at least 99%, or 100% identical to a promoter sequence in Table 4 or a functional fragment thereof.

In some embodiments, the bacterium further comprises a nucleotide sequence that encodes a PheP sequence that is at least 80%, at least 85%, at least 90%, at least 91%, at least 92%, at least 93%, at least 94%, at least 95%, at least 96%, at least 97%, at least 98%, at least 99%, or 100% identical to a PheP amino acid sequence encoded by the PheP nucleotide sequence within SEQ ID NO: 506 or a functional fragment thereof, wherein the PheP sequence is operably linked to a nucleic acid sequence that is at least 80%, at least 85%, at least 90%, at least 91%, at least 92%, at least 93%, at least 94%, at least 95%, at least 96%, at least 97%, at least 98%, at least 99%, or 100% identical to a promoter sequence in Table 5 or a functional fragment thereof.

In other embodiments, a PME, e.g., PAL, is expressed under the control of an oxygen level-dependent promoter fused to a binding site for a transcriptional activator, e.g., CRP. CRP (cyclic AMP receptor protein or catabolite activator protein or CAP) plays a major regulatory role in bacteria by repressing genes responsible for the uptake, metabolism, and assimilation of less favorable carbon sources when rapidly metabolizable carbohydrates, such as glucose, are present (Wu et al., 2015). This preference for glucose has been termed glucose repression, as well as carbon catabolite repression (Deutscher, 2008; Garke and Stülke, 2008). In some embodiments, PME, e.g., PAL, expression is controlled by an oxygen level-dependent promoter fused to a CRP binding site. In some embodiments, PAL expression is controlled by an FNR promoter fused to a CRP binding site. In these embodiments, cyclic AMP binds to CRP when no glucose is present in the environment. This binding causes a conformational change in CRP, and allows CRP to bind tightly to its binding site. CRP binding then activates transcription of the PME gene, e.g., PAL gene, by recruiting RNA polymerase to the FNR promoter via direct protein-protein interactions. In the presence of glucose, cyclic AMP does not bind to CRP and a PME, e.g., PAL, gene transcription is repressed. In some embodiments, an oxygen level-dependent promoter (e.g., an FNR promoter) fused to a binding site for a transcriptional activator is used to ensure that a PME, e.g., PAL, is not expressed under anaerobic conditions when sufficient amounts of glucose are present, e.g., by adding glucose to growth media in vitro.

In another embodiment, a PME, e.g., LAAD, is expressed under the control of an inducible promoter fused to a binding site for a transcriptional activator, e.g., CRP, such that expression is repressed in the presence of glucose.

In some embodiments, LAAD is not under the control of an FNRs promoter. LAAD requires oxygen to catalyze the degradation of phenylalanine to phenylpyruvate. Therefore, it would not be desirable to induce LAAD expression under strictly anaerobic conditions where it would be minimally active.

In some embodiments, a PME, e.g., PAL or LAAD, is expressed under the control of an inducible promoter that is responsive to specific molecules or metabolites in the environment, e.g., the mammalian gut. For example, the short-chain fatty acid propionate is a major microbial fermentation metabolite localized to the gut (Hosseini et al., 2011). In one embodiment, PAL gene expression is under the control of a propionate-inducible promoter. In a more specific embodiment, PME gene expression is under the control of a propionate-inducible promoter that is activated by the presence of propionate in the mammalian gut. Any molecule or metabolite found in the mammalian gut, in a healthy and/or disease state, may be used to induce PME gene expression. Non-limiting examples include propionate, bilirubin, aspartate aminotransferase, alanine aminotransferase, blood coagulation factors II, VII, IX, and X, alkaline phosphatase, gamma glutamyl transferase, hepatitis antigens and antibodies, alpha fetoprotein, anti-mitochondrial, smooth muscle, and anti-nuclear antibodies, iron, transferrin, ferritin, copper, ceruloplasmin, ammonia, and manganese. In alternate embodiments, PME, e.g., PAL and/or LAAD, gene expression is under the control of a P_{araBAD} promoter, which is activated in the presence of the sugar arabinose. In one embodiment, LAAD expression is under the control of the P_{araBAD} promoter. In one embodiment, expression of LAAD occurs under aerobic or microaerobic conditions.

In some embodiments, the PAL gene is expressed under the control of a promoter that is induced by exposure to tetracycline. In some embodiments, gene expression is further optimized by methods known in the art, e.g., by optimizing ribosomal binding sites, manipulating transcriptional regulators, and/or increasing mRNA stability.

In some embodiments, the genetically engineered bacteria comprise a stably maintained plasmid or chromosome carrying the PAL gene, such that PAL can be expressed in the host cell, and the host cell is capable of survival and/or growth in vitro, e.g., in medium, and/or in vivo, e.g., in the gut. In some embodiments, the genetically engineered bacteria comprise two or more distinct PAL genes. In some embodiments, the genetically engineered bacteria comprise multiple copies of the same PAL gene. In some embodiments, the PAL gene is present on a plasmid and operably linked to a directly or indirectly inducible promoter. In some embodiments, the PAL gene is present on a plasmid and operably linked to a promoter that is induced under low-oxygen or anaerobic conditions. In some embodiments, the PAL gene is present on a chromosome and operably linked to a directly or indirectly inducible promoter. In some embodiments, the PAL gene is present in the chromosome and operably linked to a promoter that is induced under low-oxygen or anaerobic conditions. In some embodiments, the PAL gene is present on a plasmid and operably linked to a promoter that is induced by exposure to tetracycline.

In some embodiments, the genetically engineered bacteria comprise a stably maintained plasmid or chromosome carrying the LAAD gene, such that LAAD can be expressed in the host cell, and the host cell is capable of survival and/or growth in vitro, e.g., in medium, and/or in vivo, e.g., in the gut. In some embodiments, the genetically engineered bacteria comprise two or more distinct LAAD genes. In some embodiments, the genetically engineered bacteria comprise multiple copies of the same LAAD gene. In some embodiments, the LAAD gene is present on a plasmid and operably linked to a directly or indirectly inducible promoter. In some embodiments, the LAAD gene is present on a plasmid and operably linked to a promoter that is inducible, e.g., by arabinose or tetracycline. In some embodiments, the LAAD gene is present on a chromosome and operably linked to a directly or indirectly inducible promoter. In some embodiments, the LAAD gene is present in the chromosome and operably linked to a promoter that is induced, e.g., by arabinose. In some embodiments, the LAAD gene is present on a plasmid and operably linked to a promoter that is induced by exposure to tetracycline.

In some embodiments, the genetically engineered bacteria comprise an oxygen-level dependent transcriptional regulator, e.g., FNR, ANR, or DNR, and corresponding promoter from a different bacterial species. The non-native oxygen-level dependent transcriptional regulator and promoter increase the transcription of genes operably linked to said promoter, e.g., PAL, in a low-oxygen or anaerobic environment, as compared to the native transcriptional regulator and promoter in the bacteria under the same conditions. In certain embodiments, the non-native oxygen-level dependent transcriptional regulator is an FNR protein from N. gonorrhoeae (see, e.g., Isabella et al., 2011). In some embodiments, the corresponding wild-type transcriptional regulator is left intact and retains wild-type activity. In alternate embodiments, the corresponding wild-type transcriptional regulator is deleted or mutated to reduce or eliminate wild-type activity.

In some embodiments, the genetically engineered bacteria comprise a wild-type oxygen-level dependent transcriptional regulator, e.g., FNR, ANR, or DNR, and corresponding promoter that is mutated relative to the wild-type promoter from bacteria of the same subtype. The mutated promoter enhances binding to the wild-type transcriptional regulator and increases the transcription of genes operably linked to said promoter, e.g., PAL, in a low-oxygen or anaerobic environment, as compared to the wild-type promoter under the same conditions. In some embodiments, the genetically engineered bacteria comprise a wild-type oxygen-level dependent promoter, e.g., FNR, ANR, or DNR promoter, and corresponding transcriptional regulator that is mutated relative to the wild-type transcriptional regulator from bacteria of the same subtype. The mutated transcriptional regulator enhances binding to the wild-type promoter and increases the transcription of genes operably linked to said promoter, e.g., PAL, in a low-oxygen or anaerobic environment, as compared to the wild-type transcriptional regulator under the same conditions. In certain embodiments, the mutant oxygen-level dependent transcriptional regulator is an FNR protein comprising amino acid substitutions that enhance dimerization and FNR activity (see, e.g., Moore et al., 2006).

In some embodiments, the genetically engineered bacteria comprise multiple copies of the endogenous gene encoding the oxygen level-sensing transcriptional regulator, e.g., the FNR gene. In some embodiments, the gene encoding the oxygen level-sensing transcriptional regulator is present on a plasmid. In some embodiments, the gene encoding the oxygen level-sensing transcriptional regulator and the gene encoding PAL are present on different plasmids. In some embodiments, the gene encoding the oxygen level-sensing transcriptional regulator and the gene encoding PAL are present on the same plasmid. In some embodiments, the gene encoding the oxygen level-sensing transcriptional regulator is present on a chromosome. In some embodiments, the gene encoding the oxygen level-sensing transcriptional regulator and the gene encoding PAL are present on different chromosomes. In some embodiments, the gene encoding the oxygen level-sensing transcriptional regulator and the gene encoding PAL are present on the same chromosome. In some instances, it may be advantageous to express the oxygen level-sensing transcriptional regulator under the control of an inducible promoter in order to enhance expression stability. In some embodiments, expression of the transcriptional regulator is controlled by a different promoter than the promoter that controls expression of the gene encoding the phenylalanine-metabolizing enzyme. In some embodiments, expression of the transcriptional regulator is controlled by the same promoter that controls expression of the phenylalanine-metabolizing enzyme. In some embodiments, the transcriptional regulator and the phenylalanine-metabolizing enzyme are divergently transcribed from a promoter region.

In some embodiments, the PME, e.g., PAL, LAAD, and/or PAH, is expressed on a low-copy plasmid. In some embodiments, the low-copy plasmid may be useful for increasing stability of expression. In some embodiments, the low-copy plasmid may be useful for decreasing leaky expression under non-inducing conditions. In some embodiments, the PME, e.g., PAL, LAAD, and/or PAH, is expressed on a high-copy plasmid. In some embodiments, the high-copy plasmid may be useful for increasing the PME, e.g., PAL, LAAD, and/or PAH, expression, thereby increasing the metabolism of phenylalanine and reducing hyperphenylalaninemia. In some embodiments, a genetically engineered bacterium comprising a the PME, e.g., PAL, LAAD, and/or PAH, expressed on a high-copy plasmid does not increase phenylalanine metabolism or decrease phenylalanine levels as compared to a genetically engineered bacterium comprising the same PME, e.g., PAL, LAAD, and/or PAH, expressed on a low-copy plasmid in the absence of heterologous pheP and additional copies of a native pheP. Genetically engineered bacteria comprising the same the PME gene, e.g., PAL, LAAD, and/or PAH gene on high and low copy plasmids were generated. For example, either PAL1 or PAL3 on a high-copy plasmid and a low-copy plasmid were generated, and each metabolized and reduced phenylalanine to similar levels. Thus, in some embodiments, the rate-limiting step of phenylalanine metabolism is phenylalanine availability. In these embodiments, it may be advantageous to increase phenylalanine transport into the cell, thereby enhancing phenylalanine metabolism. In conjunction with pheP, even low-copy PAL plasmids are capable of almost completely eliminating Phe from a test sample. Furthermore, in some embodiments, that incorporate pheP, there may be additional advantages to using a low-copy PAL-expressing plasmid in conjunction in order to enhance the stability of PAL expression while maintaining high phenylalanine metabolism, and to reduce negative selection pressure on the transformed bacterium. In alternate embodiments, the phenylalanine transporter is used in conjunction with the high-copy plasmid.

In some embodiments, a transporter may not increase phenylalanine degradation. For example, Proteus mirabilis LAAD is localized to the plasma membrane, with the enzymatic catalysis occurring in the periplasm. Phenylalanine can readily traverse the outer membrane without the need of a transporter. Therefore, in embodiments, in which the genetically engineered bacteria express LAAD, a transporter may not be needed or improve phenylalanine metabolism.

In some embodiments, the PME, e.g., PAL, LAAD, and/or PAH, gene is expressed on a chromosome. In some embodiments, expression from the chromosome may be useful for increasing stability of expression of the PME. In some embodiments, the PME gene, e.g., PAL, LAAD, and/or PAH gene(s), is integrated into the bacterial chromosome at one or more integration sites in the genetically engineered bacteria. In some embodiments, the PME gene, e.g., PAL, LAAD, and/or PAH gene(s) is inserted into the bacterial genome at one or more of the following insertion sites in E. coli Nissle: malE/K, insB/I, araC/BAD, lacZ, agal/rsml, thyA, and malP/T. Any suitable insertion site may be used. The insertion site may be anywhere in the genome, e.g., in a gene required for survival and/or growth, such as thyA (to create an auxotroph); in an active area of the genome, such as near the site of genome replication; and/or in between divergent promoters in order to reduce the risk of unintended transcription, such as between AraB and AraC of the arabinose operon. In some embodiments, more than one copy, e.g., two, three, four, five, six, seven, eight, nine, ten or more copies of the PME gene, e.g., PAL, PAH, and/or LAAD is integrated into the bacterial chromosome at one or more integration sites in the genetically engineered bacteria. The more than one copy of a PME gene may be more than one copy of the same PME gene or more than one copy of different PME genes.

In some embodiments, the bacteria are genetically engineered to include multiple mechanisms of action (MoAs), e.g., circuits producing multiple copies of the same product (e.g., to enhance copy number) or circuits performing multiple different functions. Examples of insertion sites include, but are not limited to, malE/K, yicS/nepI, insB/I, araC/BAD, lacZ, agal/rsml, thyA, malP/T, dapA, and cea, and others known in the art. For example, the genetically engineered bacteria may include four copies of PAL inserted at four different insertion sites, e.g., malE/K, insB/I araC/BAD, and lacZ. The genetically engineered bacteria may also include four copies of PAL inserted at four different insertion sites, e.g., malE/K, yicS/nepI, agaI/rsmI, and cea, and one copy of a phenylalanine transporter gene inserted at a different insertion site. Alternatively, the genetically engineered bacteria may include three copies of PAL inserted at three different insertion sites, e.g., malE/K, insB/I, and lacZ, and three copies of a phenylalanine transporter gene inserted at three different insertion sites, e.g., dapA, cea, and araC/BAD.

In some embodiments, the genetically engineered bacteria comprise one or more of (1) PAL, PAH, LAAD for degradation of phenylalanine, in wild-type or in a mutated form (for increased stability or metabolic activity) (2) transporter PheP or AroP for uptake of phenylalanine, in wild-type or in mutated form (for increased stability or metabolic activity) (3) PAL, PAH, LAAD, and/or PheP for secretion and extracellular phenylalanine degradation, (4) components of secretion machinery, as described herein (5) Auxotrophy, e.g., deltaThyA, deltaDapA (6) antibiotic resistance, including but not limited to, kanamycin or chloramphenicol resistance (7) mutations/deletions in genes involved in oxygen metabolism, as described herein and (8) mutations/deletions in genes of the endogenous Nissle phenylalanine synthesis pathway (e.g., delta PheA for Phe auxotrophy).

In one embodiment, the genetically engineered bacteria comprise one or more copies of PAL3 (e.g., under the control of a Pfnr promoter or an IPTG-inducible promoter), and one or more copies of PAL1 (e.g. under the control of a Pfnr promoter or an IPTG-inducible promoter). In one embodiment, the genetically engineered bacteria comprise one or more copies of PAL3 (e.g., under the control of a Pfnr promoter or an IPTG-inducible promoter), and one or more copies of PAL1 (e.g. under the control of a Pfnr promoter or an IPTG-inducible promoter); and further comprises one or more copies of a phenylalanine transporter (e.g., PheP and/or AroP, e.g., under the control of a Pfnr promoter or an IPTG-inducible promoter). In one embodiment, the genetically engineered bacteria comprise one or more copies of PAL3 (e.g., under the control of a Pfnr promoter or an IPTG-inducible promoter), and one or more copies of LAAD (e.g., under the control of the ParaBAD promoter). In one embodiment, the genetically engineered bacteria comprise one or more copies of PAL3 (e.g., under the control of a Pfnr promoter or an IPTG-inducible promoter), and one or more copies of LAAD (e.g., under the control of the ParaBAD promoter); and further comprises one or more copies of a phenylalanine transporter (e.g., PheP and/or AroP, e.g., under the control of a Pfnr promoter or an IPTG-inducible promoter). In one embodiment, the genetically engineered bacteria comprise one or more copies of PAL3 (e.g., under the control of a Pfnr promoter or an IPTG-inducible promoter), and one or more copies of PAH. In one embodiment, the genetically engineered bacteria comprise one or more copies of PAL3 (e.g., under the control of a Pfnr promoter or an IPTG-inducible promoter), and one or more copies of PAH; and further comprises one or more copies of a phenylalanine transporter (e.g., PheP and/or AroP, e.g., under the control of a Pfnr promoter or an IPTG-inducible promoter). In one embodiment, the genetically engineered bacteria comprise one or more copies of PAL1 (e.g., under the control of a Pfnr promoter or an IPTG-inducible promoter), and one or more copies of LAAD (e.g., under the control of the ParaBAD promoter). In one embodiment, the genetically engineered bacteria comprise one or more copies of PAL1 (e.g., under the control of a Pfnr promoter or an IPTG-inducible promoter), and one or more copies of LAAD (e.g., under the control of the ParaBAD promoter); and further comprises one or more copies of a phenylalanine transporter (e.g., PheP and/or AroP, e.g., under the control of a Pfnr promoter or an IPTG-inducible promoter). In one embodiment, the genetically engineered bacteria comprise one or more copies of PAL1 (e.g., under the control of a Pfnr promoter or an IPTG-inducible promoter) and one or more copies of PAH. In one embodiment, the genetically engineered bacteria comprise one or more copies of PAL1 (e.g., under the control of a Pfnr promoter or an IPTG-inducible promoter) and one or more copies of PAH; and further comprises one or more copies of a phenylalanine transporter (e.g., PheP and/or AroP, e.g., under the control of a Pfnr promoter or an IPTG-inducible promoter). In one embodiment, the genetically engineered bacteria comprise one or more copies of PAH and one or more copies of LAAD (e.g., under the control of the ParaBAD promoter). In one embodiment, the genetically engineered bacteria comprise one or more copies of PAH and one or more copies of LAAD (e.g., under the control of the ParaBAD promoter); and further comprises one or more copies of a phenylalanine transporter (e.g., PheP and/or AroP, e.g., under the control of a Pfnr promoter or an IPTG-inducible promoter). PMEs and transporters may be integrated into any of the insertion sites described herein.

In one embodiment, the genetically engineered bacteria comprise one or more copies of PAL3 (e.g., under the control of a Pfnr promoter or an IPTG-inducible promoter), one or more copies of LAAD (e.g., under the control of the ParaBAD promoter), and one or more copies of PAH. In one embodiment, the genetically engineered bacteria comprise one or more copies of PAL3 (e.g., under the control of a Pfnr promoter or an IPTG-inducible promoter), one or more copies of LAAD (e.g., under the control of the ParaBAD promoter), and one or more copies of PAH; and further comprise one or more copies of a phenylalanine transporter (e.g., PheP and/or AroP, e.g., under the control of a Pfnr promoter or an IPTG-inducible promoter). In one embodiment, the genetically engineered bacteria comprise one or more copies of PAL3 (e.g., under the control of a Pfnr promoter or an IPTG-inducible promoter), one or more copies of LAAD (e.g., under the control of the ParaBAD promoter), and one or more copies of PAL1 (e.g., under the control of a Pfnr promoter or an IPTG-inducible promoter). In one embodiment, the genetically engineered bacteria comprise one or more copies of PAL3 (e.g., under the control of a Pfnr promoter or an IPTG-inducible promoter), one or more copies of LAAD (e.g., under the control of the ParaBAD promoter), and one or more copies of PAL1 (e.g., under the control of a Pfnr promoter or an IPTG-inducible promoter); and further comprise one or more copies of a phenylalanine transporter (e.g., PheP and/or AroP, e.g., under the control of a Pfnr promoter or an IPTG-inducible promoter). In one embodiment, the genetically engineered bacteria comprise one or more copies of PAL3 (e.g., under the control of a Pfnr promoter or an IPTG-inducible promoter), one or more copies of PAL1 (e.g., under the control of a Pfnr promoter or an IPTG-inducible promoter), and one or more copies of PAH. In one embodiment, the genetically engineered bacteria comprise one or more copies of PAL3 (e.g., under the control of a Pfnr promoter or an IPTG-inducible promoter), one or more copies of PAL1 (e.g., under the control of a Pfnr promoter or an IPTG-inducible promoter), and one or more copies of PAH; and further comprise one or more copies of a phenylalanine transporter (e.g., PheP and/or AroP, e.g., under the control of a Pfnr promoter or an IPTG-inducible promoter). In one embodiment, the genetically engineered bacteria comprise one or more copies of LAAD (e.g., under the control of the ParaBAD promoter), one or more copies of PAH, and one or more copies of PAL1 (e.g., under the control of an Pfnr promoter or an IPTG-inducible promoter). In one embodiment, the genetically engineered bacteria comprise one or more copies of LAAD (e.g., under the control of the ParaBAD promoter), one or more copies of PAH, and one or more copies of PAL1 (e.g., under the control of an Pfnr promoter or an IPTG-inducible promoter); and further comprise one or more copies of a phenylalanine transporter (e.g., PheP and/or AroP, e.g., under the control of a Pfnr promoter or an IPTG-inducible promoter). PMEs and/or transporters may be integrated into any of the insertion sites described herein. Alternatively, PMEs and/or transporters may be comprised on low or high copy plasmids. PMEs and/or transporters may be integrated into any of the insertion sites described herein in combination with PMEs and/or transporters that are comprised on low or high copy plasmids.

In one embodiment, the genetically engineered bacteria comprise one or more copies of PAL3 (e.g., under the control of a Pfnr promoter or an IPTG-inducible promoter), one or more copies of PAL1, (e.g., under the control of a Pfnr promoter or an IPTG-inducible promoter), one or more copies of LAAD (e.g., under the control of the ParaBAD promoter), and one or more copies of PAH. In one embodiment, the genetically engineered bacteria comprise one or more copies of PAL3 (e.g., under the control of a Pfnr promoter or an IPTG-inducible promoter), one or more copies of PAL1, (e.g., under the control of a Pfnr promoter or an IPTG-inducible promoter), one or more copies of LAAD (e.g., under the control of the ParaBAD promoter), and one or more copies of PAH; and further comprise one or more copies of a phenylalanine transporter (e.g., PheP and/or AroP, e.g., under the control of a Pfnr promoter or an IPTG-inducible promoter). PMEs and transporters may be integrated into any of the insertion sites described herein. Alternatively, PMEs and/or transporters may be comprised on low or high copy plasmids.

In one embodiment, the genetically engineered bacteria comprise one copy of PAL (e.g., PAL1 or PAL3, e.g., under the control of a Pfnr promoter or an IPTG-inducible promoter), one copy of PheP (e.g., under the control of a Pfnr promoter or an IPTG-inducible promoter), and one copy of LAAD (e.g., under the control of the ParaBAD promoter). In one embodiment, the genetically engineered bacteria comprise one copy of PAL (e.g., PAL1 or PAL3, e.g., under the control of a Pfnr promoter or an IPTG-inducible promoter), two copies of PheP (e.g., under the control of a Pfnr promoter or an IPTG-inducible promoter), and one copy of LAAD (e.g., under the control of the ParaBAD promoter). In one embodiment, the genetically engineered bacteria comprise one copy of PAL (e.g., PAL1 or PAL3, e.g., under the control of a Pfnr promoter or an IPTG-inducible promoter), one copy of PheP (e.g., under the control of a Pfnr promoter or an IPTG-inducible promoter), and two copies of LAAD (e.g., under the control of the ParaBAD promoter). In one embodiment, the genetically engineered bacteria comprise one copy of PAL (e.g., PAL1 or PAL3, e.g., under the control of a Pfnr promoter or an IPTG-inducible promoter), two copies of PheP (e.g., under the control of a Pfnr promoter or an IPTG-inducible promoter), and two copies of LAAD (e.g., under the control of the ParaBAD promoter). PMEs and transporters may be integrated into any of the insertion sites described herein. Alternatively, located PMEs and/or transporters may be comprised on low or high copy plasmids.

In one embodiment, the genetically engineered bacteria comprise two copies of PAL (e.g., PAL1 or PAL3, e.g., under the control of a Pfnr promoter or an IPTG-inducible promoter), one copy of PheP (e.g., under the control of a Pfnr promoter or an IPTG-inducible promoter), and one copy of LAAD (e.g., under the control of the ParaBAD promoter). In one embodiment, the genetically engineered bacteria comprise two copies of PAL (e.g., PAL1 or PAL3, e.g., under the control of a Pfnr promoter or an IPTG-inducible promoter), two copies of PheP (e.g., under the control of a Pfnr promoter or an IPTG-inducible promoter), and one copy of LAAD (e.g., under the control of the ParaBAD promoter). In one embodiment, the genetically engineered bacteria comprise two copies of PAL (e.g., PAL1 or PAL3, e.g., under the control of a Pfnr promoter or an IPTG-inducible promoter), one copy of PheP (e.g., under the control of a Pfnr promoter or an IPTG-inducible promoter), and two copies of LAAD (e.g., under the control of the ParaBAD promoter). In one embodiment, the genetically engineered bacteria comprise two copies of PAL (e.g., PAL1 or PAL3, e.g., under the control of a Pfnr promoter or an IPTG-inducible promoter), two copies of PheP (e.g., under the control of a Pfnr promoter or an IPTG-inducible promoter), and two copies of LAAD (e.g., under the control of the ParaBAD promoter).

In one embodiment, the genetically engineered bacteria comprise three copies of PAL (e.g., PAL1 or PAL3, e.g., under the control of a Pfnr promoter or an IPTG-inducible promoter), one copy of PheP (e.g., under the control of a Pfnr promoter or an IPTG-inducible promoter), and one copy of LAAD (e.g., under the control of the ParaBAD promoter). In one embodiment, the genetically engineered bacteria comprise three copies of PAL (e.g., PAL1 or PAL3, e.g., under the control of a Pfnr promoter or an IPTG-inducible promoter), two copies of PheP (e.g., under the control of a Pfnr promoter or an IPTG-inducible promoter), and one copy of LAAD (e.g., under the control of the ParaBAD promoter). In one embodiment, the genetically engineered bacteria comprise three copies of PAL (e.g., PAL1 or PAL3, e.g., under the control of a Pfnr promoter or an IPTG-inducible promoter), one copy of PheP (e.g., under the control of a Pfnr promoter or an IPTG-inducible promoter), and two copies of LAAD (e.g., under the control of the ParaBAD promoter). In one embodiment, the genetically engineered bacteria comprise three copies of PAL (e.g., PAL1 or PAL3, e.g., under the control of a Pfnr promoter or an IPTG-inducible promoter), two copies of PheP (e.g., under the control of a Pfnr promoter or an IPTG-inducible promoter), and two copies of LAAD (e.g., under the control of the ParaBAD promoter). In one embodiment, the genetically engineered bacteria comprise three copies of PAL (e.g., PAL1 or PAL3, e.g., under the control of a Pfnr promoter or an IPTG-inducible promoter), three copies of PheP (e.g., under the control of a Pfnr promoter or an IPTG-inducible promoter), and two copies of LAAD (e.g., under the control of the ParaBAD promoter). In one embodiment, the genetically engineered bacteria comprise three copies of PAL (e.g., PAL1 or PAL3, e.g., under the control of a Pfnr promoter or an IPTG-inducible promoter), three copies of PheP (e.g., under the control of a Pfnr promoter or an IPTG-inducible promoter), and one copy of LAAD (e.g., under the control of the ParaBAD promoter).

In one embodiment, the genetically engineered bacteria comprise four copies of PAL (e.g., PAL1 or PAL3, e.g., under the control of a Pfnr promoter or an IPTG-inducible promoter), one copy of PheP (e.g., under the control of a Pfnr promoter or an IPTG-inducible promoter), and one copy of LAAD (e.g., under the control of the ParaBAD promoter). In one embodiment, the genetically engineered bacteria comprise four copies of PAL (e.g., PAL1 or PAL3, e.g., under the control of a Pfnr promoter or an IPTG-inducible promoter), two copies of PheP (e.g., under the control of a Pfnr promoter or an IPTG-inducible promoter), and one copy of LAAD (e.g., under the control of the ParaBAD promoter). In one embodiment, the genetically engineered bacteria comprise four copies of PAL (e.g., PAL1 or PAL3, e.g., under the control of a Pfnr promoter or an IPTG-inducible promoter), one copy of PheP (e.g., under the control of a Pfnr promoter or an IPTG-inducible promoter), and two copies of LAAD (e.g., under the control of the ParaBAD promoter). In one embodiment, the genetically engineered bacteria comprise four copies of PAL (e.g., PAL1 or PAL3, e.g., under the control of a Pfnr promoter or an IPTG-inducible promoter), two copies of PheP (e.g., under the control of a Pfnr promoter or an IPTG-inducible promoter), and two copies of LAAD (e.g., under the control of the ParaBAD promoter).

In one embodiment, the genetically engineered bacteria comprise five copies of PAL (e.g., PAL1 or PAL3, e.g., under the control of a Pfnr promoter or an IPTG-inducible promoter), one copy of PheP (e.g., under the control of a Pfnr promoter or an IPTG-inducible promoter), and one copy of LAAD (e.g., under the control of the ParaBAD promoter). In one embodiment, the genetically engineered bacteria comprise five copies of PAL (e.g., PAL1 or PAL3, e.g., under the control of a Pfnr promoter or an IPTG-inducible promoter), two copies of PheP (e.g., under the control of a Pfnr promoter or an IPTG-inducible promoter), and one copy of LAAD (e.g., under the control of the ParaBAD promoter). In one embodiment, the genetically engineered bacteria comprise five copies of PAL (e.g., PAL1 or PAL3, e.g., under the control of a Pfnr promoter or an IPTG-inducible promoter), one copy of PheP (e.g., under the control of a Pfnr promoter or an IPTG-inducible promoter), and two copies of LAAD (e.g., under the control of the ParaBAD promoter). In one embodiment, the genetically engineered bacteria comprise five copies of PAL (e.g., PAL1 or PAL3, e.g., under the control of a Pfnr promoter or an IPTG-inducible promoter), two copies of PheP (e.g., under the control of a Pfnr promoter or an IPTG-inducible promoter), and two copies of LAAD (e.g., under the control of the ParaBAD promoter).

In one embodiment, the genetically engineered bacteria comprise one or more PMEs for metabolizing phenylalanine in combination with one or more PMEs for secretion. In one embodiment, the genetically engineered bacteria comprise one or more PMEs for metabolizing phenylalanine and a phenylalanine transporter in combination with one or more PMEs for secretion. In one embodiment, the genetically engineered bacteria comprise one or more PMEs for metabolizing phenylalanine and a phenylalanine transporter in combination with one or more PMEs for secretion, and also include an auxotrophy and/or an antibiotic resistance. Secretion systems described herein are utilized to secrete the PMEs in the genetically engineered bacteria with multiple mechanisms of action.

In one embodiment, the genetically engineered bacteria comprise two additional copies of PheP (in addition to the wild-type gene). This provides redundancy, in case one of the PheP genes acquires a mutation. In one embodiment, the PheP genes are inserted at lacZ and agal/rsml. In one embodiment, the two copies of PheP are under the control of the PfnrS promoter. In one embodiment, the genetically engineered bacteria comprise three copies of PAL3. In one embodiment, the genetically engineered bacteria comprise three copies of PAL3, inserted at malEK, malPT, yicS/nepl. In one embodiment, the expression of the three copies of PAL3 is under the control of the PfnrS promoter. In one embodiment, the genetically engineered bacteria comprise one or more copies of LAAD. In one embodiment, the genetically engineered bacteria comprise one copy of LAAD, inserted in the arabinose operon. In one embodiment, LAAD is under the control of the endogenous ParaBAD promoter. In one embodiment, the genetically engineered bacteria comprise an auxotrophy, e.g., deltaThyA. In one embodiment, the genetically engineered bacteria comprise an antibiotic resistance. In one embodiment the genetically engineered bacteria comprise an antibiotic resistance and an auxotrophy, e.g., deltaThyA. In one embodiment, the genetically engineered bacteria do not comprise an auxotrophy, e.g., deltaThyA. In one embodiment, the genetically engineered bacteria do not comprise an antibiotic resistance. In one embodiment the genetically engineered bacteria comprise neither an antibiotic resistance nor an auxotrophy, e.g., deltaThyA.

In one embodiment, the genetically engineered bacteria comprise three copies of PAL, e.g., PAL3, 2 copies of PheP (in addition to the endogenous PheP), and one copy of LAAD. In one embodiment, the genetically engineered bacteria comprise three copies of PAL, e.g., PAL3, 2 copies of PheP (in addition to the endogenous PheP), and one copy of LAAD, and an auxotrophy, e.g., delta ThyA. In one embodiment, the genetically engineered bacteria comprise three copies of PAL, 2 copies of PheP (in addition to the endogenous PheP), and one copy of LAAD, and an antibiotic resistance gene. In one embodiment, the genetically engineered bacteria comprise three copies of PAL, 2 copies of PheP (in addition to the endogenous PheP), and one copy of LAAD, and an antibiotic resistance gene and an auxotrophy, e.g., delta ThyA.

In one embodiment, the genetically engineered bacteria comprise three copies of PAL (each under control of a PfnrS promoter), 2 copies of PheP (each under control of a PfnrS promoter), and one copy of LAAD (under the control of the endogenous ParaBAD promoter). In one embodiment, the genetically engineered bacteria comprise three copies of PAL (each under control of a PfnrS promoter), 2 copies of PheP (each under control of a PfnrS promoter), and one copy of LAAD (under the control of the endogenous ParaBAD promoter), and an antibiotic resistance. In one embodiment, the genetically engineered bacteria comprise three copies of PAL (each under control of a PfnrS promoter), 2 copies of PheP (each under control of a PfnrS promoter), and one copy of LAAD (under the control of the endogenous ParaBAD promoter), and an auxotrophy, e.g., delta ThyA. In one embodiment, the genetically engineered bacteria comprise three copies of PAL (each under control of a PfnrS promoter), 2 copies of PheP (each under control of a PfnrS promoter), and one copy of LAAD (under the control of the endogenous ParaBAD promoter), and an antibiotic resistance and an auxotrophy, e.g., deltaThyA.

In one embodiment, the genetically engineered bacteria comprise three copies of PAL (each under control of a PfnrS promoter and inserted at the malEK, malPT, and yicS/nepl sites), 2 copies of PheP (each under control of a PfnrS promoter and inserted at the LacZ and agal/rsml sites), and one copy of LAAD (under the control of the endogenous ParaBAD promoter, and inserted in the endogenous arabinose operon). In one embodiment, the genetically engineered bacteria comprise three copies of PAL (each under control of a PfnrS promoter and inserted at the malEK, malPT, and yicS/nepl sites), 2 copies of PheP (each under control of a PfnrS promoter and inserted at the LacZ and agal/rsml sites), and one copy of LAAD (under the control of the endogenous ParaBAD promoter, and inserted in the endogenous arabinose operon), and further comprise an antibiotic resistance. In one embodiment, the genetically engineered bacteria comprise three copies of PAL (each under control of a PfnrS promoter and inserted at the malEK, malPT, and yicS/nepl sites), 2 copies of PheP (each under control of a PfnrS promoter and inserted at the LacZ and agal/rsml sites), and one copy of LAAD (under the control of the endogenous ParaBAD promoter, and inserted in the endogenous arabinose operon) and further comprise an auxotrophy, e.g., deltaThyA. In one embodiment, the genetically engineered bacteria comprise three copies of PAL (each under control of a PfnrS promoter and inserted at the malEK, malPT, and yicS/nepl sites), 2 copies of PheP (each under control of a PfnrS promoter and inserted at the LacZ and agal/rsml sites), and one copy of LAAD (under the control of the endogenous ParaBAD promoter, and inserted in the endogenous arabinose operon), and further comprise an antibiotic resistance and an auxotrophy, e.g., deltaThyA.

In one embodiment, the genetically engineered bacteria comprise four copies of PAL, e.g., PAL3, one copy of PheP (in addition to the endogenous PheP), and one copy of LAAD. In one embodiment, the genetically engineered bacteria comprise four copies of PAL, e.g., PAL3, one copy of PheP (in addition to the endogenous PheP), and one copy of LAAD, and an auxotrophy, e.g., delta ThyA. In one embodiment, the genetically engineered bacteria comprise four copies of PAL, one copy of PheP (in addition to the endogenous PheP), and one copy of LAAD, and an antibiotic resistance gene. In one embodiment, the genetically engineered bacteria comprise four copies of PAL, one copy of PheP (in addition to the endogenous PheP), and one copy of LAAD, and an antibiotic resistance gene and an auxotrophy, e.g., delta ThyA.

In one embodiment, the genetically engineered bacteria comprise four copies of PAL (each under control of an IPTG-inducible promoter), one copy of PheP (under control of an IPTG-inducible promoter), and one copy of LAAD (under the control of the endogenous ParaBAD promoter). In one embodiment, the genetically engineered bacteria comprise four copies of PAL (each under control of an IPTG-inducible promoter), one copy of PheP (under control of an IPTG-inducible promoter), and one copy of LAAD (under the control of the endogenous ParaBAD promoter), and an antibiotic resistance. In one embodiment, the genetically engineered bacteria comprise four copies of PAL (each under control of an IPTG-inducible promoter), one copy of PheP (under control of an IPTG-inducible promoter), and one copy of LAAD (under the control of the endogenous ParaBAD promoter), and an auxotrophy, e.g., delta ThyA. In one embodiment, the genetically engineered bacteria comprise four copies of PAL (each under control of an IPTG-inducible promoter), one copy of PheP (under control of an IPTG-inducible promoter), and one copy of LAAD (under the control of the endogenous ParaBAD promoter), and an antibiotic resistance and an auxotrophy, e.g., deltaThyA. In some embodiments, the genetically engineered bacteria comprise one or more E. coli Nissle bacteriophage sequence(s), and at least one of the bacteriophage sequence(s) is mutated or modified, e.g., to delete the bacteriophage sequence, e.g., an endogenous prophage sequence, in part or whole. In some embodiments, the deletion prevents the bacteria from being able to express infectious bacteriophage particles. Non-limiting examples of such mutations or modifications are described in PCT/US2018/038840, the contents of which are incorporated by reference in their entirety. In some embodiments, the genetically engineered bacteria comprise one or modifications or mutations in one or more of Phage 1, 2 or 3 as described in PCT/US2018/038840 (WO2018237198A1). In some embodiments, the genetically engineered bacteria comprise a modification or mutation in Phage 3. In some embodiments, the mutations include deletions, insertions, substitutions and inversions and are located in or encompass one or more Phage 3 genes. In some embodiments, the one or more insertions comprise an antibiotic cassette. In some embodiments, the mutation is a deletion. In some embodiments, the genetically engineered bacteria comprise one or more deletions, which are located in or comprise one or more genes selected from ECOLIN_09965, ECOLIN_09970, ECOLIN_09975, ECOLIN_09980, ECOLIN_09985, ECOLIN_09990, ECOLIN_09995, ECOLIN_10000, ECOLIN_10005, ECOLIN_10010, ECOLIN_10015, ECOLIN_10020, ECOLIN_10025, ECOLIN_10030, ECOLIN_10035, ECOLIN_10040, ECOLIN_10045, ECOLIN_10050, ECOLIN_10055, ECOLIN_10065, ECOLIN_10070, ECOLIN_10075, ECOLIN_10080, ECOLIN_10085, ECOLIN_10090, ECOLIN_10095, ECOLIN_10100, ECOLIN_10105, ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, ECOLIN_10170, ECOLIN_10175, ECOLIN_10180, ECOLIN_10185, ECOLIN_10190, ECOLIN_10195, ECOLIN_10200, ECOLIN_10205, ECOLIN_10210, ECOLIN_10220, ECOLIN_10225, ECOLIN_10230, ECOLIN_10235, ECOLIN_10240, ECOLIN_10245, ECOLIN_10250, ECOLIN_10255, ECOLIN_10260, ECOLIN_10265, ECOLIN_10270, ECOLIN_10275, ECOLIN_10280, ECOLIN_10290, ECOLIN_10295, ECOLIN_10300, ECOLIN_10305, ECOLIN_10310, ECOLIN_10315, ECOLIN_10320, ECOLIN_10325, ECOLIN_10330, ECOLIN_10335, ECOLIN_10340, and ECOLIN_10345. In one embodiment, the genetically engineered bacteria comprise a complete or partial deletion of one or more of ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, ECOLIN_10170, and ECOLIN_10175. In one specific embodiment, the deletion is a complete deletion of ECOLIN_10110, ECOLIN_10115, ECOLIN_10120, ECOLIN_10125, ECOLIN_10130, ECOLIN_10135, ECOLIN_10140, ECOLIN_10145, ECOLIN_10150, ECOLIN_10160, ECOLIN_10165, and ECOLIN_10170, and a partial deletion of ECOLIN_10175. In one embodiment, the sequence of SEQ ID NO: 292 or SEQ ID NO: 414 is deleted from the Phage 3 genome (see, e.g., SEQ ID NO: 130 or SEQ ID NO: 281 from PCT/US2018/038840, WO2018237198, the contents of which are hereby incorporated in their entireties). In one embodiment, a sequence comprising SEQ ID NO: 292 or SEQ ID NO: 414 is deleted from the Phage 3 genome (see, e.g., SEQ ID NO: 130 or SEQ ID NO: 281 from PCT/US2018/038840, WO2018237198, the contents of which are hereby incorporated in their entireties). Exemplary engineered bacteria comprising modified phage are disclosed in PCT/US2018/038840 (WO2018237198), the contents of which are hereby incorporated by reference.

In some embodiments, the engineered bacterium further comprises a modified pks island (colibactin island). Non-limiting examples are described in PCT/US2021/061579, the contents of which are herein incorporated by reference in their entirety. Colibactin is a cyclomodulin that is synthetized by enzymes encoded by the pks genomic island. See Fais 2018. The pks genomic island is “highly conserved” in Enterobacteriaceae. Id. In Escherichia coli, a 54-kilobase pks genomic island contains 19 genes, clbA to clbS, and encodes various enzymes that have been described as an “assembly line responsible for colibactin synthesis.” Id. The pks genomic island assembly line for colibactin synthesis includes three polyketide synthases (ClbC, ClbI, ClbO), three non-ribosomal peptide synthases (ClbH, ClbJ, ClbN), two hybrid non-ribosomal peptide/polyketide synthases (ClbB, ClbK), and nine accessory, tailoring, and editing proteins. The polyketide synthases, non-ribosomal peptide synthases, and hybrid enzymes “are usually organized in mega-complexes as an assembly line, in which the synthesized compound is transferred from one enzymatic module to the following one.” Id. Colibactin undergoes a prodrug activation mechanism that incorporates an N-terminal structural motif, which is removed during the final stage of biosynthesis.

In some embodiments, the engineered microorganism. e.g., engineered bacterium, comprises a modified pks island (colibactin island). In some embodiments, the engineered microorganism. e.g., engineered bacterium, comprises a modified clb sequence selected from one or more of the clbA, clbB, clbC, clbD, clbE, clbF, clbG, clbH, clbI, clbJ, clbK, clbL clbM, clbN, clbO, clbP, clbQ, clbR, and clbS gene sequences, as compared to a suitable control, e.g., the native pks island in an unmodified bacterium of the same strain and/or subtype. In some embodiments, the modified clb sequence is an insertion, a substitution, and/or a deletion as compared to the control. In some embodiments, the modified clb sequence is a deletion of the clb island, e.g., clbA, clbB, clbC, clbD, clbE, clbF, clbG, clbH, clbI, clbJ, clbK, clbL clbM, clbN, clbO, clbP, clbQ, clbR, and clbS. In one embodiment, the colibactin deletion is the whole island except for the clbS gene, e.g., a deletion of clbA, clbB, clbC, clbD, clbE, clbF, clbG, clbH, clbI, clbJ, clbK, clbL clbM, clbN, clbO, clbP, clbQ, and clbR.

In some embodiments, the modified endogenous colibactin island comprises one or more modified clb sequences selected from clbA (SEQ ID NO: 294), clbB (SEQ ID NO: 295), clbC (SEQ ID NO: 296), clbD (SEQ ID NO: 297), clbE (SEQ ID NO: 298), clbF (SEQ ID NO: 299), clbG (SEQ ID NO: 300), clbH (SEQ ID NO: 301), clbI (SEQ ID NO: 302), clbJ (SEQ ID NO: 303), clbK (SEQ ID NO: 304), clbL (SEQ ID NO: 305), clbM (SEQ ID NO: 306), clbN (SEQ ID NO: 307), clbO (SEQ ID NO: 308), clbP (SEQ ID NO: 309), clbQ (SEQ ID NO: 310), clbR (SEQ ID NO: 311), or clbS (SEQ ID NO: 312) gene. In some embodiments, the modified endogenous colibactin island comprises a deletion of clbA (SEQ ID NO: 294), clbB (SEQ ID NO: 295), clbC (SEQ ID NO: 296), clbD (SEQ ID NO: 297), clbE (SEQ ID NO: 298), clbF (SEQ ID NO: 299), clbG (SEQ ID NO: 300), clbH (SEQ ID NO: 301), clbI (SEQ ID NO: 302), clbJ (SEQ ID NO: 303), clbK (SEQ ID NO: 304, clbL (SEQ ID NO: 305), clbM (SEQ ID NO: 306), clbN (SEQ ID NO: 307), clbO (SEQ ID NO: 308), clbP (SEQ ID NO: 309), clbQ (SEQ ID NO: 310), and clbR (SEQ ID NO: 311).

### Pharmaceutical Compositions and Formulations

Pharmaceutical compositions comprising the genetically engineered bacteria disclosed herein may be used to treat, manage, ameliorate, and/or prevent diseases associated with hyperphenylalaninemia, e.g., PKU. Pharmaceutical compositions comprising one or more genetically engineered bacteria, alone or in combination with prophylactic agents, therapeutic agents, and/or and pharmaceutically acceptable carriers are provided. In certain embodiments, the pharmaceutical composition comprises one species, strain, or subtype of bacteria that are engineered to comprise the genetic modifications described herein. In alternate embodiments, the pharmaceutical composition comprises two or more species, strains, and/or subtypes of bacteria that are each engineered to comprise the genetic modifications described herein.

The pharmaceutical compositions described herein may be formulated in a conventional manner using one or more physiologically acceptable carriers comprising excipients and auxiliaries, which facilitate processing of the active ingredients into compositions for pharmaceutical use. Methods of formulating pharmaceutical compositions are known in the art (see, e.g., “Remington's Pharmaceutical Sciences,” Mack Publishing Co., Easton, PA). In some embodiments, the pharmaceutical compositions are subjected to tabletting, lyophilizing, direct compression, conventional mixing, dissolving, granulating, levigating, emulsifying, encapsulating, entrapping, or spray drying to form tablets, granulates, nanoparticles, nanocapsules, microcapsules, microtablets, pellets, or powders, which may be enterically coated or uncoated. Appropriate formulation depends on the route of administration.

The genetically engineered bacteria described herein may be formulated into pharmaceutical compositions in any suitable dosage form (e.g., liquids, capsules, sachet, hard capsules, soft capsules, tablets, enteric coated tablets, suspension powders, granules, or matrix sustained release formations for oral administration) and for any suitable type of administration (e.g., oral, topical, injectable, immediate-release, pulsatile-release, delayed-release, or sustained release).

Suitable dosage amounts for the genetically engineered bacteria may range from about 10⁵ to 10¹² bacteria, e.g., about 10⁵ bacteria, about 10⁶ bacteria, about 10⁷ bacteria, about 10⁸ bacteria, about 10⁹ bacteria, about 10¹⁰ bacteria, about 10¹¹ bacteria, or about 10¹¹ bacteria, e.g., bacterial cells. The composition may be administered once or more daily, weekly, or monthly.

In some embodiments, pharmaceutical composition comprises about 1×10¹¹, about 2×10¹¹, about 3×10¹¹, about 4×10¹¹, about 5×10¹¹, about 6×10¹¹, about 7×10¹¹, about 8×10¹¹, or about 9×10¹¹ of the genetically engineered bacteria, e.g., bacterial cells, disclosed herein as determined by live cell counting.

In some embodiments, pharmaceutical composition comprises about 1×10¹², about 2×10¹², about 3×10¹², about 4×10¹², about 5×10¹², about 6×10¹², about 7×10¹², about 8×10¹², or about 9×10¹² of the genetically engineered bacteria, e.g., bacterial cells, disclosed herein as determined by live cell counting.

In some embodiments, pharmaceutical composition comprises about 1×10¹², about 1.1×10¹², about 1.2×10¹², about 1.3×10¹², about 1.4×10¹², about 1.5×10¹², about 1.6×10¹², about 1.7×10¹², about 1.8×10¹², about 1.9×10¹², about 2×10¹², about 2.1×10¹², about 2.2×10¹², about 2.3×10¹², about 2.4×10¹², about 2.5×10¹², about 2.6×10¹², about 2.7×10¹², about 2.8×10¹², about 2.9×10¹², or about 3×10¹² of the genetically engineered bacteria, e.g., bacterial cells, disclosed herein as determined by live cell counting.

In some embodiments, the method comprises administering to the subject genetically engineered bacteria at a dose of 1×10¹¹, 2×10¹¹, 3×10¹¹, 4×10¹¹, 5×10¹¹, 6×10¹¹, 7×10¹¹, 8×10¹¹, or 9×10¹¹ bacterial cells, as determined by live cell counting. In some embodiments, the method comprises administering to the subject genetically engineered bacteria at a dose of 1×10¹², 2×10¹², 3×10¹², 4×10¹², 5×10¹², 6×10¹², 7×10¹², 8×10¹², or 9×10¹² bacterial cells, as determined by live cell counting.

In some embodiments, the method comprises administering to the subject genetically engineered bacteria at a dose of about 1×10¹¹, about 2×10¹¹, about 3×10¹¹, about 4×10¹¹, about 5×10¹¹, about 6×10¹¹, about 7×10¹¹, about 8×10¹¹, or about 9×10¹¹ bacterial cells, as determined by live cell counting. In some embodiments, the method comprises administering to the subject genetically engineered bacteria at a dose of about 1×10¹², about 2×10¹², about 3×10¹², about 4×10¹², about 5×10¹², about 6×10¹², about 7×10¹², about 8×10¹², or about 9×10¹² bacterial cells, as determined by live cell counting.

In some embodiments, the method comprises administering to the subject genetically engineered bacteria at a dose of 1×10¹¹ to 2×10¹¹, 2×10¹¹ to 3×10¹¹, 3×10¹¹ to 4×10¹¹, 4×10¹¹ to 5×10¹¹, 5×10¹¹ to 6×10¹¹, 6×10¹¹ to 7×10¹¹, 7×10¹¹ to 8×10¹¹, or 8×10¹¹ to 9×10¹¹ bacterial cells, as determined by live cell counting. In some embodiments, the method comprises administering to the subject genetically engineered bacteria at a dose of 1×10¹² to 2×10¹², 2×10¹² to 3×10¹², 3×10¹² to 4×10¹², 4×10¹² to 5×10¹², 5×10¹² to 6×10¹², 6×10¹² to 7×10¹², 7×10¹² to 8×10¹², or 8×10¹² to 9×10¹² bacterial cells, as determined by live cell counting.

In some embodiments, the method comprises administering to the subject genetically engineered bacteria using a dose ramp (e.g., multiple escalating doses), which may be beneficial for tolerability.

In some embodiments, the genetically engineered bacteria may be formulated into a pharmaceutical composition which comprises an agent which can neutralize stomach acidity, such as bicarbonate.

In some embodiments, the genetically engineered bacteria may be formulated into pharmaceutical compositions comprising sucralose, sodium bicarbonate, and/or a flavoring agent.

In some embodiments, the genetically engineered bacteria are dosed between 1×10¹² to 2×10¹² bacterial cells and formulated into pharmaceutical compositions comprising sucralose, sodium bicarbonate, and a flavoring agent. In these embodiments, the formulation comprises about 0.5 gram to about 3.5 grams of the genetically engineered bacteria; about 0.001 grams to about 0.1 grams of sucralose; about 0.5 gram to about 3.5 grams of sodium bicarbonate; and/or about 0.1 grams to about 1 gram of flavoring agent.

The composition may be administered before, during, or following a meal. In one embodiment, the pharmaceutical composition is administered before the subject eats a meal. In one embodiment, the pharmaceutical composition is administered currently with a meal. In one embodiment, the pharmaceutical composition is administered after the subject eats a meal.

The genetically engineered bacteria may be formulated into pharmaceutical compositions comprising one or more pharmaceutically acceptable carriers, thickeners, diluents, buffers, buffering agents, surface active agents, neutral or cationic lipids, lipid complexes, liposomes, penetration enhancers, carrier compounds, and other pharmaceutically acceptable carriers or agents. For example, the pharmaceutical composition may include, but is not limited to, the addition of calcium bicarbonate, sodium bicarbonate, calcium phosphate, various sugars and types of starch, cellulose derivatives, gelatin, vegetable oils, polyethylene glycols, and surfactants, including, for example, polysorbate 20. In some embodiments, the genetically engineered bacteria may be formulated in a solution of sodium bicarbonate, e.g., 1 molar solution of sodium bicarbonate (to buffer an acidic cellular environment, such as the stomach, for example). The genetically engineered bacteria may be administered and formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The genetically engineered bacteria disclosed herein may be administered orally and formulated as tablets, pills, dragees, capsules, liquids, gels, syrups, slurries, suspensions, etc. Pharmacological compositions for oral use can be made using a solid excipient, optionally grinding the resulting mixture, and processing the mixture of granules, after adding suitable auxiliaries if desired, to obtain tablets or dragee cores. Suitable excipients include, but are not limited to, fillers such as sugars, including lactose, sucrose, mannitol, or sorbitol; cellulose compositions such as maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP) or polyethylene glycol (PEG). Disintegrating agents may also be added, such as cross-linked polyvinylpyrrolidone, agar, alginic acid or a salt thereof such as sodium alginate.

Tablets or capsules can be prepared by conventional means with pharmaceutically acceptable excipients such as binding agents (e.g., pregelatinised maize starch, polyvinylpyrrolidone, hydroxypropyl methylcellulose, carboxymethylcellulose, polyethylene glycol, sucrose, glucose, sorbitol, starch, gum, kaolin, and tragacanth); fillers (e.g., lactose, microcrystalline cellulose, or calcium hydrogen phosphate); lubricants (e.g., calcium, aluminum, zinc, stearic acid, polyethylene glycol, sodium lauryl sulfate, starch, sodium benzoate, L-leucine, magnesium stearate, talc, or silica); disintegrants (e.g., starch, potato starch, sodium starch glycolate, sugars, cellulose derivatives, silica powders); or wetting agents (e.g., sodium lauryl sulphate). The tablets may be coated by methods well known in the art. A coating shell may be present, and common membranes include, but are not limited to, polylactide, polyglycolic acid, polyanhydride, other biodegradable polymers, alginate-polylysine-alginate (APA), alginate-polymethylene-co-guanidine-alginate (A-PMCG-A), hydroymethylacrylate-methyl methacrylate (HEMA-MMA), multilayered HEMA-MMA-MAA, polyacrylonitrilevinylchloride (PAN-PVC), acrylonitrile/sodium methallylsulfonate (AN-69), polyethylene glycol/poly pentamethylcyclopentasiloxane/polydimethylsiloxane (PEG/PD5/PDMS), poly N,N-dimethyl acrylamide (PDMAAm), siliceous encapsulates, cellulose sulphate/sodium alginate/polymethylene-co-guanidine (CS/A/PMCG), cellulose acetate phthalate, calcium alginate, k-carrageenan-locust bean gum gel beads, gellan-xanthan beads, poly(lactide-co-glycolides), carrageenan, starch poly-anhydrides, starch polymethacrylates, polyamino acids, and enteric coating polymers.

In some embodiments, the genetically engineered bacteria are enterically coated for release into the gut or a particular region of the gut, for example, the large intestine. The typical pH profile from the stomach to the colon is about 14 (stomach), about 5.5-6 (duodenum), about 7.3-8.0 (ileum), and about 5.5-6.5 (colon). In some diseases, the pH profile may be modified. In some embodiments, the coating is degraded in specific pH environments in order to specify the site of release. In some embodiments, at least two coatings are used. In some embodiments, the outside coating and the inside coating are degraded at different pH levels.

Liquid preparations for oral administration may take the form of solutions, syrups, suspensions, or a dry product for constitution with water or other suitable vehicle before use. Such liquid preparations may be prepared by conventional means with pharmaceutically acceptable agents such as suspending agents (e.g., sorbitol syrup, cellulose derivatives, or hydrogenated edible fats); emulsifying agents (e.g., lecithin or acacia); non-aqueous vehicles (e.g., almond oil, oily esters, ethyl alcohol, or fractionated vegetable oils); and preservatives (e.g., methyl or propyl-p-hydroxybenzoates or sorbic acid). The preparations may also contain buffer salts, flavoring, coloring, and sweetening agents as appropriate. Preparations for oral administration may be suitably formulated for slow release, controlled release, or sustained release of the genetically engineered bacteria described herein.

In one embodiment, the genetically engineered bacteria of the disclosure may be formulated in a composition suitable for administration to pediatric subjects. As is well known in the art, children differ from adults in many aspects, including different rates of gastric emptying, pH, gastrointestinal permeability, etc. (Ivanovska et al., 2014). Moreover, pediatric formulation acceptability and preferences, such as route of administration and taste attributes, are critical for achieving acceptable pediatric compliance. Thus, in one embodiment, the composition suitable for administration to pediatric subjects may include easy-to-swallow or dissolvable dosage forms, or more palatable compositions, such as compositions with added flavors, sweeteners, or taste blockers. In one embodiment, a composition suitable for administration to pediatric subjects may also be suitable for administration to adults.

In one embodiment, the composition suitable for administration to pediatric subjects may include a solution, syrup, suspension, elixir, powder for reconstitution as suspension or solution, dispersible/effervescent tablet, chewable tablet, gummy candy, lollipop, freezer pop, troche, chewing gum, oral thin strip, orally disintegrating tablet, sachet, soft gelatin capsule, sprinkle oral powder, or granules. In one embodiment, the composition is a gummy candy, which is made from a gelatin base, giving the candy elasticity, desired chewy consistency, and longer shelf-life. In some embodiments, the gummy candy may also comprise sweeteners or flavors.

In one embodiment, the composition suitable for administration to pediatric subjects may include a flavor. As used herein, “flavor” is a substance (liquid or solid) that provides a distinct taste and aroma to the formulation. Flavors also help to improve the palatability of the formulation. Flavors include, but are not limited to, strawberry, vanilla, lemon, grape, bubble gum, and cherry.

In certain embodiments, the genetically engineered bacteria may be orally administered, for example, with an inert diluent or an assimilable edible carrier. The compound may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound by other than parenteral administration, it may be necessary to coat the compound with, or co-administer the compound with, a material to prevent its inactivation.

In another embodiment, the pharmaceutical composition comprising the recombinant bacteria may be a comestible product, for example, a food product. In one embodiment, the food product is milk, concentrated milk, fermented milk (yogurt, sour milk, frozen yogurt, lactic acid bacteria-fermented beverages), milk powder, ice cream, cream cheeses, dry cheeses, soybean milk, fermented soybean milk, vegetable-fruit juices, fruit juices, sports drinks, confectionery, candies, infant foods (such as infant cakes), nutritional food products, animal feeds, or dietary supplements. In one embodiment, the food product is a fermented food, such as a fermented dairy product. In one embodiment, the fermented dairy product is yogurt. In another embodiment, the fermented dairy product is cheese, milk, cream, ice cream, milk shake, or kefir. In another embodiment, the recombinant bacteria are combined in a preparation containing other live bacterial cells intended to serve as probiotics. In another embodiment, the food product is a beverage. In one embodiment, the beverage is a fruit juice-based beverage or a beverage containing plant or herbal extracts. In another embodiment, the food product is a jelly or a pudding. Other food products suitable for administration of the recombinant bacteria are well known in the art. See, e.g., US 2015/0359894 and US 2015/0238545, the entire contents of each of which are expressly incorporated herein by reference. In yet another embodiment, the pharmaceutical composition is injected into, sprayed onto, or sprinkled onto a food product, such as bread, yogurt, or cheese.

In some embodiments, the composition is formulated for intraintestinal administration, intrajejunal administration, intraduodenal administration, intraileal administration, gastric shunt administration, or intracolic administration, via nanoparticles, nanocapsules, microcapsules, or microtablets, which are enterically coated or uncoated. The pharmaceutical compositions may also be formulated in rectal compositions such as suppositories or retention enemas, using, e.g., conventional suppository bases such as cocoa butter or other glycerides. The compositions may be suspensions, solutions, or emulsions in oily or aqueous vehicles, and may contain suspending, stabilizing and/or dispersing agents.

The genetically engineered bacteria described herein may be administered intranasally, formulated in an aerosol form, spray, mist, or in the form of drops, and conveniently delivered in the form of an aerosol spray presentation from pressurized packs or a nebuliser, with the use of a suitable propellant (e.g., dichlorodifluoromethane, trichlorofluoromethane, dichlorotetrafluoroethane, carbon dioxide or other suitable gas). Pressurized aerosol dosage units may be determined by providing a valve to deliver a metered amount. Capsules and cartridges (e.g., of gelatin) for use in an inhaler or insufflator may be formulated containing a powder mix of the compound and a suitable powder base such as lactose or starch.

The genetically engineered bacteria may be administered and formulated as depot preparations. Such long acting formulations may be administered by implantation or by injection, including intravenous injection, subcutaneous injection, local injection, direct injection, or infusion. For example, the compositions may be formulated with suitable polymeric or hydrophobic materials (e.g., as an emulsion in an acceptable oil) or ion exchange resins, or as sparingly soluble derivatives (e.g., as a sparingly soluble salt).

In some embodiments, disclosed herein are pharmaceutically acceptable compositions in single dosage forms. Single dosage forms may be in a liquid or a solid form. Single dosage forms may be administered directly to a patient without modification or may be diluted or reconstituted prior to administration. In certain embodiments, a single dosage form may be administered in bolus form, e.g., single injection, single oral dose, including an oral dose that comprises multiple tablets, capsule, pills, etc. In alternate embodiments, a single dosage form may be administered over a period of time, e.g., by infusion.

Single dosage forms of the pharmaceutical composition may be prepared by portioning the pharmaceutical composition into smaller aliquots, single dose containers, single dose liquid forms, or single dose solid forms, such as tablets, granulates, nanoparticles, nanocapsules, microcapsules, microtablets, pellets, or powders, which may be enterically coated or uncoated. A single dose in a solid form may be reconstituted by adding liquid, typically sterile water or saline solution, prior to administration to a patient.

In other embodiments, the composition can be delivered in a controlled release or sustained release system. In one embodiment, a pump may be used to achieve controlled or sustained release. In another embodiment, polymeric materials can be used to achieve controlled or sustained release of the therapies of the present disclosure (see, e.g., U.S. Pat. No. 5,989,463). Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. The polymer used in a sustained release formulation may be inert, free of leachable impurities, stable on storage, sterile, and biodegradable. In some embodiments, a controlled or sustained release system can be placed in proximity of the prophylactic or therapeutic target, thus requiring only a fraction of the systemic dose. Any suitable technique known to one of skill in the art may be used.

Dosage regimens may be adjusted to provide a therapeutic response. Dosing can depend on several factors, including severity and responsiveness of the disease, route of administration, time course of treatment (days to months to years), and time to amelioration of the disease. For example, a single bolus may be administered at one time, several divided doses may be administered over a predetermined period of time, or the dose may be reduced or increased as indicated by the therapeutic situation. The specification for the dosage is dictated by the unique characteristics of the active compound and the particular therapeutic effect to be achieved. Dosage values may vary with the type and severity of the condition to be alleviated. For any particular subject, specific dosage regimens may be adjusted over time according to the individual need and the professional judgment of the treating clinician. Toxicity and therapeutic efficacy of compounds provided herein can be determined by standard pharmaceutical procedures in cell culture or animal models. For example, LD₅₀, ED₅₀, EC₅₀, and IC₅₀ may be determined, and the dose ratio between toxic and therapeutic effects (LD₅₀/ED₅₀) may be calculated as the therapeutic index. Compositions that exhibit toxic side effects may be used, with careful modifications to minimize potential damage to reduce side effects. Dosing may be estimated initially from cell culture assays and animal models. The data obtained from in vitro and in vivo assays and animal studies can be used in formulating a range of dosage for use in humans.

The ingredients are supplied either separately or mixed together in unit dosage form, for example, as a dry lyophilized powder or water-free concentrate in a hermetically sealed container such as an ampoule or sachet indicating the quantity of active agent. If the mode of administration is by injection, an ampoule of sterile water for injection or saline can be provided so that the ingredients may be mixed prior to administration.

The pharmaceutical compositions may be packaged in a hermetically sealed container such as an ampoule or sachet indicating the quantity of the agent. In one embodiment, one or more of the pharmaceutical compositions is supplied as a dry sterilized lyophilized powder or water-free concentrate in a hermetically sealed container and can be reconstituted (e.g., with water or saline) to the appropriate concentration for administration to a subject. In an embodiment, one or more of the prophylactic or therapeutic agents or pharmaceutical compositions is supplied as a dry sterile lyophilized powder in a hermetically sealed container stored between 2° C. and 8° C. and administered within 1 hour, within 3 hours, within 5 hours, within 6 hours, within 12 hours, within 24 hours, within 48 hours, within 72 hours, or within one week after being reconstituted. Cryoprotectants can be included for a lyophilized dosage form, principally 0-10% sucrose (optimally 0.5-1.0%). Other suitable cryoprotectants include trehalose and lactose. Other suitable bulking agents include glycine and arginine, either of which can be included at a concentration of 0-0.05%, and polysorbate-80 (optimally included at a concentration of 0.005-0.01%). Additional surfactants include but are not limited to polysorbate 20 and BRU surfactants. The pharmaceutical composition may be prepared as an injectable solution and can further comprise an agent useful as an adjuvant, such as those used to increase absorption or dispersion, e.g., hyaluronidase.

In some embodiments, the genetically engineered bacteria may be formulated in a composition comprising trehalose. In some embodiments, the genetically engineered bacteria may be formulated in a composition comprising 5, 6, 7, 8, 9, 10, 11, 12, 13, 14, or 15% w/v trehalose. In some embodiments, the genetically engineered bacteria may be formulated in a composition comprising 10% w/v trehalose. In some embodiments, the genetically engineered bacteria may be formulated in a composition comprising 10-100 mM, 20-50 mM, 30-50 mM, 40-50 mM, 40-60 mM, 40-70 mM, 50-60 mM, 50-70 mM, or 50-80 mM Tris. In some embodiments, the genetically engineered bacteria may be formulated in a composition comprising about 20 mM, about 30 mM, about 40 mM, about 50 mM, about 60 mM, about 70 mM, or about 80 mM Tris. In some embodiments, the genetically engineered bacteria may be formulated in a composition comprising 50 mM Tris. In some embodiments, the genetically engineered bacteria may be formulated in a composition comprising 50 mM Tris, at about pH 7.5 buffer. In some embodiments, the genetically engineered bacteria may be formulated in a composition comprising Tris, e.g., 20 mM, 30 mM, 40 mM, 50 mM, 60 mM, 70 mM, or 80 mM Tris and trehalose, e.g., 5, 6, 7, 8, 9, 10, 11, 12, 13, 14, or 15% w/v trehalose. In some embodiments, the genetically engineered bacteria may be formulated in a composition comprising 50 mM Tris, pH 7.5 buffer containing 10% w/v trehalose. In some embodiments, this composition may be administered as a frozen liquid. In some embodiments, this composition may be subsequently lyophilized. In some embodiments, this composition may lyophilized for oral suspension. In some embodiments, this lyophilized composition may be formulated into a pharmaceutical composition comprising sodium bicarbonate and a flavoring agent.

### REFERENCES

Am, P. H. 2014. Phenylketonuria (PKU) (Elsevier Inc).
Anikster Y, Haack T B, Vilboux T, et al. Biallelic mutations in DNAJC12 cause hyperphenylalaninemia, dystonia, and intellectual disability. Am J Hum Genet. 2017; 100:257-66.
Blau N, Martinez A, Hoffmann G F, Thöny B. DNAJC12 deficiency: A new strategy in the diagnosis of hyperphenylalaninemias. Mol Genet Metab. 2018; 123:1-5.
Blau, Nenad, Julia B Hennermann, Ulrich Langenbeck, and Uta Lichter-Konecki. 2011. ‘Diagnosis, classification, and genetics of phenylketonuria and tetrahydrobiopterin (BH4) deficiencies’, Molecular genetics and metabolism, 104: S2-S9.
Blau, N., and N. Longo. 2015. ‘Alternative therapies to address the unmet medical needs of patients with phenylketonuria’, Expert Opin Pharmacother, 16: 791-800.
Blau, Nenad, Julia B Hennermann, Ulrich Langenbeck, and Uta Lichter-Konecki. 2011. ‘Diagnosis, classification, and genetics of phenylketonuria and tetrahydrobiopterin (BH4) deficiencies’, Molecular genetics and metabolism, 104: S2-S9.
de Groot, M. J., M. Hoeksma, N. Blau, D. J. Reijngoud, and F. J. van Spronsen. 2010. ‘Pathogenesis of cognitive dysfunction in phenylketonuria: review of hypotheses’, Mol Genet Metab, 99 Suppl 1: S86-9.
Foreman, P. K., A. V. Margulis, K. Alexander, R. Shediac, B. Calingaert, A. Harding, M. Pladevall-Vila, and S. Landis. 2021. ‘Birth prevalence of phenylalanine hydroxylase deficiency: a systematic literature review and meta-analysis’, Orphanet J Rare Dis, 16: 253.
Procopio, Daniela, Italia Mascaro, Stefania Ferraro, Ferdinando Ceravolo, Maria Teresa Moricca, Vincenzo Salpietro, Agata Polizzi, Martino Ruggieri, Giuseppe Bonapace, and Daniela Concolino. 2016. ‘Hyperphenylalaninemia: From Diagnosis to Therapy’, Journal of Pediatric Biochemistry, 6: 011-18.
Shoraka, H. R., A. A. Haghdoost, M. R. Baneshi, Z. Bagherinezhad, and F. Zolala. 2020. ‘Global prevalence of classic phenylketonuria based on Neonatal Screening Program Data: systematic review and meta-analysis’, Clin Exp Pediatr, 63: 34-43.
van Spronsen, F. J., A. M. van Wegberg, K. Ahring, A. Belanger-Quintana, N. Blau, A. M. Bosch, A. Burlina, J. Campistol, F. Feillet, M. Gizewska, S. C. Huijbregts, S. Kearney, V. Leuzzi, F. Maillot, A. C. Muntau, F. K. Trefz, M. van Rijn, J. H. Walter, and A. MacDonald. 2017. ‘Key European guidelines for the diagnosis and management of patients with phenylketonuria’, Lancet Diabetes Endocrinol, 5: 743-56.
Williams, D. V., B. Barua, and N. H. Andersen. 2008. ‘Hyperstable miniproteins: additive effects of D- and L-Ala mutations’, Org Biomol Chem, 6: 4287-9.

### EXAMPLES

The following examples provide illustrative embodiments of the disclosure. One of ordinary skill in the art will recognize the numerous modifications and variations that may be performed without altering the spirit or scope of the disclosure. Such modifications and variations are encompassed within the scope of the disclosure. The Examples do not in any way limit the disclosure.

### Example 1. Construction of SYNB1618

SYNB1618 was engineered with two chromosomally integrated copies of pheP and three copies of stlA under the regulatory control of the anaerobic-inducible promoter P_{fnrS}. See, e.g., Isabella et al., Development of a synthetic live bacterial therapeutic for the human metabolic disease phenylketonuria, Nature Biotechnology (2018), which is incorporated by reference in its entirety herein. The P_{fnrS} promoter was inactive in the presence of oxygen and was activated under anaerobic or microaerobic conditions by the anoxic-sensing transcriptional activator FNR. P_{frnS}-GFP transcriptional fusion in E. coli Nissle was used to confirm the activation of this promoter following oral administration in C57BL/6 mice and recovery from the gastrointestinal (GI) tract. SYNB1618 was also engineered so that Phe-degrading genes could be activated during manufacturing. Two additional copies of stlA were placed under the control of the Ptac promoter, which allowed induction by isopropyl β-d-1-thiogalactopyranoside (IPTG) in vitro.

PAL activity in SYNB1618 was constructed with genetic redundancy. Multiple copies of stlA and pheP were added to act as a genetic buffer to ensure that a loss-of-function mutation in an individual gene copy during manufacturing would not compromise strain activity. Gene insertions were also made in the same orientation in the chromosome, and in locations such that the intervening sequence between each insertion contained essential genes. This ensured that homologous recombination between duplicate genes would be lethal, thereby preventing genetic selection and propagation of mutant strains during large-scale growth.

SYNB1618 contains a copy of pma under the control of the arabinose-inducible P_{BAD} promoter. Induction of LAAD was negligible in the uninduced state, but could reach maximal activity following induction at the end of the manufacturing process; thus, a single gene copy was deemed to be sufficient. The LAAD activity present at the time of dosing is envisioned as a mechanism to capitalize on the available oxygen in the proximal GI tract, whereas PAL activity will predominate as cells pass through the more anoxic GI environments encountered distally, owing to its de novo synthesis by the P_{fnrS} promoter.

### Example 2. Construction of SYNB1934

To facilitate inducible production of PAL in Escherichia coli Nissle, the PAL gene and transcriptional and translational elements were synthesized and cloned into vector pBR322. In some embodiments, the PAL gene comprises mutations in one or more amino acid positions selected from S92G, H133M, I167K, L432I, and V470A compared to positions in wild-type PAL, e.g., Photorhabdus luminescens PAL, e.g., SEQ ID NO: 500. A mutant referred to herein as “mPAL1” (SEQ ID NO: 501; Table 6) was generated according to the methods provided herein. The bacterium referred to herein as SYNB1934 comprises mPAL1. In some embodiments, the mutant PAL comprises mutations in one or more amino acid positions selected from S92G, H133F, A433S, and V470A compared to positions in wild-type PAL, e.g., Photorhabdus luminescens PAL, e.g., SEQ ID NO: 500. A mutant referred to herein as “mPAL2” (SEQ ID NO: 502; Table 6) was generated according to the methods provided herein. In some embodiments, the mutant PAL comprises mutations in one or more amino acid positions selected from S92G, H133F, A263T, K366K (e.g., silent mutation in polynucleotide sequence), L396L (e.g., silent mutation in polynucleotide sequence), and V470A compared to positions in wild-type PAL, e.g., Photorhabdus luminescens PAL, e.g., SEQ ID NO: 500. A mutant referred to herein as “mPAL3” (SEQ ID NO: 503; Table 6) was generated according to the methods provided herein. Exemplary PAL mutants are known in the art and disclosed herein. See, e.g., PCT/US2021/023003 (WO2021188819A1), PCT/US2021/063976 (WO2022146718A1), U.S. 63/132,627, the contents of which are hereby incorporated by reference.

Each of the plasmids described herein was transformed into E. coli Nissle for the studies described herein according to the following steps. All tubes, solutions, and cuvettes were pre-chilled to 4° C. An overnight culture of E. coli Nissle was diluted 1:100 in 5 mL of lysogeny broth (LB) containing ampicillin and grown until it reached an OD₆₀₀ of 0.4-0.6. The E. coli cells were then centrifuged at 2,000 rpm for 5 min at 4° C., the supernatant was removed, and the cells were resuspended in 1 mL of 4° C. water. The E. coli were again centrifuged at 2,000 rpm for 5 min at 4° C., the supernatant was removed, and the cells were resuspended in 0.5 mL of 4° C. water. The E. coli were again centrifuged at 2,000 rpm for 5 min at 4° C., the supernatant was removed, and the cells were finally resuspended in 0.1 mL of 4° C. water. The electroporator was set to 2.5 kV. Plasmid (0.5 μg) was added to the cells, mixed by pipetting, and pipetted into a sterile, chilled cuvette. The dry cuvette was placed into the sample chamber, and the electric pulse was applied. One mL of room-temperature SOC media was added immediately, and the mixture was transferred to a culture tube and incubated at 37° C. for 1 hr. The cells were spread out on an LB plate containing ampicillin and incubated overnight.

To facilitate inducible production of mutant, mPAL1, mPAL2 and mPAL3 were cloned into low copy plasmids (pSC101 origin of replication) under control of an anhydrous tetracycline (aTc)-responsive promoter and transferred to Nissle bacteria.

Bacterial strains containing different genes integrated directly into the E. coli chromosome were constructed including: lacI-Ptac-pheP is integrated at the rhtBC locus, and/or Pbad-LAAD is integrated at the araBC locus, and/or lacI-Ptac-mPAL1 is integrated at multiple sites (Table 8). These strains also contain two chromosomal deletions (1) a 9 kilobase (kb) pair segment of an endogenous prophage sequence, Φ, which prevents the cells from being able to express infectious bacteriophage particles and (2) dapA, which renders the strain an auxotroph as described herein. The methods described below may be used for engineering bacterial strains comprising chromosomal insertions (e.g., the integrated strains listed below in Table 8).

TABLE 8
Strains with lacI-P_{tac}-mPAL1 integrated
at multiple chromosomal sites

| Strain | Copies of mPAL1 | Integration Sites of mPAL1 |
|---|---|---|
| 7369 | 1 | exo/cea |
| 7380 | 2 | exo/cea, malEK |
| 7487 | 4 | exo/cea, malEK, agaI/rsmI, yicS/nepI |
| 7502 | 4 | exo/cea, malEK, agaI/rsmI, yicS/nepI |
| 7701 | 4 | exo/cea, malEK, agaI/rsmI, yicS/nepI |
| (SYNB1934) | | |
| 7393 | 3* | exo/cea, malEK |
| 7488 | 4* | exo/cea, malEK, agaI/rsmI, |
| 7504 | 4* | exo/cea, malEK, yicS/nepI |

*lacI-Ptac-mPAL1-mPAL1 integrated at malEK site

The SYN-PKU7369 strain (rhtBC::lacI-Ptac-pheP; exo/cea::lacI-Ptac-mPAL1) contains a copy of mPAL1 integrated at the exo/cea locus and a copy of pheP integrated at the rhtBC locus, with both genes operatively linked to separate copies of the synthetic IPTG inducible promoter, Ptac, and transcribed independently from each chromosomal site. A copy of the transcriptional repressor, lacI, was included in the integration construct of both pheP and mPAL1, divergently transcribed from both pheP and mPAL1 as shown herein. Sequences of exemplary constructs are shown below in which the pheP and the mPAL1 genes are independently transcribed under the control of separate synthetic IPTG inducible promoters, Ptac, each of which also contain lacI, which is divergently transcribed and under control of its constitutive promoter. Nucleotide sequences in bold designate the IPTG inducible Ptac promoter, nucleotide sequences in italics designate either pheP or mPAL, and underlined nucleotide sequences designate lacI and its constitutive promoter. To prevent unwanted homologous recombination in iterative rounds of integration, the mPAL1 sequences was codon optimized.

Nucleotide sequence of pheP integration construct
(SEQ ID NO: 506)
TCACTGCCCGCTTTCCAGTCGGGAAACCTGTCGTGCCAGCTGCATTAATGAATCGGCCAA
CGCGCGGGGAGAGGCGGTTTGCGTATTGGGCGCCAGGGTGGTTTTTCTTTTCACCAGTGA
GACTGGCAACAGCTGATTGCCCTTCACCGCCTGGCCCTGAGAGAGTTGCAGCAAGCGGT
CCACGCTGGTTTGCCCCAGCAGGCGAAAATCCTGTTTGATGGTGGTTAACGGCGGGATAT
AACATGAGCTATCTTCGGTATCGTCGTATCCCACTACCGAGATATCCGCACCAACGCGCA
GCCCGGACTCGGTAATGGCGCGCATTGCGCCCAGCGCCATCTGATCGTTGGCAACCAGC
ATCGCAGTGGGAACGATGCCCTCATTCAGCATTTGCATGGTTTGTTGAAAACCGGACATG
GCACTCCAGTCGCCTTCCCGTTCCGCTATCGGCTGAATTTGATTGCGAGTGAGATATTTAT
GCCAGCCAGCCAGACGCAGACGCGCCGAGACAGAACTTAATGGGCCCGCTAACAGCGCG
ATTTGCTGGTGACCCAATGCGACCAGATGCTCCACGCCCAGTCGCGTACCGTCCTCATGG
GAGAAAATAATACTGTTGATGGGTGTCTGGTCAGAGACATCAAGAAATAACGCCGGAAC
ATTAGTGCAGGCAGCTTCCACAGCAATGGCATCCTGGTCATCCAGCGGATAGTTAATGAT
CAGCCCACTGACGCGTTGCGCGAGAAGATTGTGCACCGCCGCTTTACAGGCTTCGACGCC
GCTTCGTTCTACCATCGACACCACCACGCTGGCACCCAGTTGATCGGCGCGAGATTTAAT
CGCCGCGACAATTTGCGACGGCGCGTGCAGGGCCAGACTGGAGGTGGCAACGCCAATCA
GCAACGACTGTTTGCCCGCCAGTTGTTGTGCCACGCGGTTGGGAATGTAATTCAGCTCCG
CCATCGCCGCTTCCACTTTTTCCCGCGTTTTCGCAGAAACGTGGCTGGCCTGGTTCACCAC
GCGGGAAACGGTCTGATAAGAGACACCGGCATACTCTGCGACATCGTATAACGTTACTG
GTTTCATATTCACCACCCTGAATTGACTCTCTTCCGGGCGCTATCATGCCATACCGCGAA
AGGTTTTGCGCCATTCGATGGCGCGCCGCTTCGTCAGGCCACATAGCTTTCTTGTTCTGAT
CGGAACGATCGTTGGCTGTGTTGACAATTAATCATCGGCTCGTATAATGTGTGGAATT
GTGAGCGCTCACAATTAGCTGTCACCGGATGTGCTTTCCGGTCTGATGAGTCCGTG
AGGACGAAACAGCCTCTACAAATAATTTTGTTTAATAGCTTCGGTAAGTTTACGGAG
GATTTGTCATGAAAAACGCTAGTACGGTAAGTGAGGATACAGCCTCGAACCAAGAACCAACGT
TGCACCGTGGGTTGCACAACCGTCACATCCAGCTTATCGCACTTGGCGGCGCTATTGGAACGG
GGTTATTCCTTGGAATTGGACCGGCCATTCAAATGGCCGGGCCAGCGGTTTTGTTGGGGTATG
GTGTAGCGGGGATCATTGCATTTCTTATTATGCGTCAGTTGGGAGAAATGGTGGTTGAAGAACC
TGTATCCGGCAGCTTCGCGCATTTCGCGTACAAGTATTGGGGTCCATTTGCTGGCTTTCTGAGT
GGCTGGAATTATTGGGTGATGTTTGTCCTGGTTGGAATGGCGGAACTTACTGCGGCCGGCATT
TATATGCAGTACTGGTTTCCTGATGTGCCTACGTGGATCTGGGCCGCGGCTTTCTTTATTATTAT
CAATGCAGTCAATCTGGTCAACGTGCGCTTGTATGGTGAGACGGAGTTCTGGTTCGCATTAATT
AAGGTATTAGCTATTATCGGAATGATTGGCTTTGGGTTATGGTTGCTGTTTAGCGGGCACGGCG
GTGAGAAAGCCTCTATCGATAACCTTTGGCGCTACGGTGGGTTCTTTGCTACAGGATGGAACG
GGTTAATCTTGAGTCTTGCGGTCATCATGTTCAGTTTTGGTGGCCTTGAATTGATTGGTATCACG
GCAGCAGAGGCGCGTGACCCAGAAAAAAGCATCCCCAAAGCCGTTAATCAGGTGGTGTACCG
CATCTTATTATTTTACATTGGTTCACTGGTCGTGTTGTTGGCTCTGTACCCATGGGTTGAGGTGA
AATCTAACTCATCCCCCTTCGTCATGATCTTTCATAACCTTGATTCAAATGTGGTCGCCAGCGCG
TTAAACTTTGTAATCCTGGTGGCAAGCCTTTCCGTGTACAATTCAGGGGTCTATTCTAATAGTCG
TATGTTGTTCGGGCTTTCGGTCCAAGGAAACGCGCCGAAATTCCTGACACGCGTTAGTCGTCGT
GGTGTGCCCATTAATAGCCTGATGCTGAGTGGTGCAATCACTTCTTTAGTCGTGCTTATTAACTA
TTTACTGCCTCAGAAGGCATTCGGGTTATTAATGGCTTTAGTTGTCGCAACGTTATTGTTAAACT
GGATCATGATCTGTTTAGCACACCTGCGTTTCCGTGCGGCTATGCGTCGCCAGGGTCGTGAAA
CCCAGTTCAAGGCCTTACTTTATCCCTTTGGTAATTACTTGTGCATTGCATTTTTAGGCATGATTT
TACTGCTGATGTGTACTATGGATGATATGCGCCTGTCCGCAATCCTTTTACCCGTCTGGATTGTT
TTTCTTTTTATGGCATTCAAAACACTTCGTCGCAAGTAA
Nucleotide sequence of mPALI integration construct
(SEQ ID NO: 507)
TCACTGCCCGCTTTCCAGTCGGGAAACCTGTCGTGCCAGCTGCATTAATGAATCGGCCAA
CGCGCGGGGAGAGGCGGTTTGCGTATTGGGCGCCAGGGTGGTTTTTCTTTTCACCAGTGA
GACTGGCAACAGCTGATTGCCCTTCACCGCCTGGCCCTGAGAGAGTTGCAGCAAGCGGT
CCACGCTGGTTTGCCCCAGCAGGCGAAAATCCTGTTTGATGGTGGTTAACGGCGGGATAT
AACATGAGCTATCTTCGGTATCGTCGTATCCCACTACCGAGATATCCGCACCAACGCGCA
GCCCGGACTCGGTAATGGCGCGCATTGCGCCCAGCGCCATCTGATCGTTGGCAACCAGC
ATCGCAGTGGGAACGATGCCCTCATTCAGCATTTGCATGGTTTGTTGAAAACCGGACATG
GCACTCCAGTCGCCTTCCCGTTCCGCTATCGGCTGAATTTGATTGCGAGTGAGATATTTAT
GCCAGCCAGCCAGACGCAGACGCGCCGAGACAGAACTTAATGGGCCCGCTAACAGCGCG
ATTTGCTGGTGACCCAATGCGACCAGATGCTCCACGCCCAGTCGCGTACCGTCCTCATGG
GAGAAAATAATACTGTTGATGGGTGTCTGGTCAGAGACATCAAGAAATAACGCCGGAAC
ATTAGTGCAGGCAGCTTCCACAGCAATGGCATCCTGGTCATCCAGCGGATAGTTAATGAT
CAGCCCACTGACGCGTTGCGCGAGAAGATTGTGCACCGCCGCTTTACAGGCTTCGACGCC
GCTTCGTTCTACCATCGACACCACCACGCTGGCACCCAGTTGATCGGCGCGAGATTTAAT
CGCCGCGACAATTTGCGACGGCGCGTGCAGGGCCAGACTGGAGGTGGCAACGCCAATCA
GCAACGACTGTTTGCCCGCCAGTTGTTGTGCCACGCGGTTGGGAATGTAATTCAGCTCCG
CCATCGCCGCTTCCACTTTTTCCCGCGTTTTCGCAGAAACGTGGCTGGCCTGGTTCACCAC
GCGGGAAACGGTCTGATAAGAGACACCGGCATACTCTGCGACATCGTATAACGTTACTG
GTTTCATATTCACCACCCTGAATTGACTCTCTTCCGGGCGCTATCATGCCATACCGCGAA
AGGTTTTGCGCCATTCGATGGCGCGCCGCTTCGTCAGGCCACATAGCTTTCTTGTTCTGAT
CGGAACGATCGTTGGCTGTGTTGACAATTAATCATCGGCTCGTATAATGTGTGGAATT
GTGAGCGCTCACAATTAGCTGTCACCGGATGTGCTTTCCGGTCTGATGAGTCCGTG
AGGACGAAACAGCCTCTACAAATAATTTTGTTTAATAGCTTCGGTAAGTTTACGGAG
GATTTGTCATGAAAGCTAAAGATGTTCAGCCAACCATTATTATTAATAAAAATGGCCTTATCTCT
TTGGAAGATATCTATGACATTGCGATAAAACAAAAAAAAGTAGAAATATCAACGGAGATCACTGA
ACTTTTGACGCATGGTCGTGAAAAATTAGAGGAAAAATTAAATTCAGGAGAGGTTATATATGGAA
TCAATACAGGATTTGGAGGGAATGCCAATTTAGTTGTGCCATTTGAGAAAATCGCAGAGCATCA
GCAAAATCTGTTAACTTTTCTTGGCGCTGGTACTGGGGACTATATGTCCAAACCTTGTATTAAAG
CGTCACAATTTACTATGTTACTTTCTGTTTGCAAAGGTTGGTCTGCAACCAGACCAATTGTCGCT
CAAGCAATTGTTGATATGATTAATCATGACATTGTTCCTCTGGTTCCTCGCTATGGCTCAGTGGG
TGCAAGCGGTGATTTAATTCCTTTATCTTATATTGCACGAGCATTATGTGGTAAGGGCAAAGTTT
ATTATATGGGCGCAGAAATTGACGCTGCTGAAGCAATTAAACGTGCAGGGTTGACACCATTATC
GTTAAAAGCCAAAGAAGGTCTTGCTCTGATTAACGGCACCCGGGTAATGTCAGGAATCAGTGCA
ATCACCGTCATTAAACTGGAAAAACTATTTAAAGCCTCAATTTCTGCGATTGCCCTTGCTGTTGA
AGCATTACTTGCATCTCATGAACATTATGATGCCCGGATTCAACAAGTAAAAAATCATCCTGGTC
AAAACGCGGTGGCAAGTGCATTGCGTAATTTATTGGCAGGTTCAACGCAGGTTAATCTATTATCT
GGGGTTAAAGAACAAGCCAATAAAGCTTGTCGTCATCAAGAAATTACCCAACTAAATGATACCTT
ACAGGAAGTTTATTCAATTCGCTGTGCACCACAAGTATTAGGTATAGTGCCAGAATCTTTAGCTA
CCGCTCGGAAAATATTGGAACGGGAAGTTATCTCAGCTAATGATAATCCATTGATAGATCCAGA
AAATGGCGATGTTCTACACGGTGGAAATTTTATGGGGCAATATGTCGCCCGAACAATGGATGCA
TTAAAACTGGATATTGCTTTAATTGCCAATCATCTTCACGCCATTGTGGCTCTTATGATGGATAAC
CGTTTCTCTCGTGGATTACCTAATTCACTGAGTCCGACACCCGGCATGTATCAAGGTTTTAAAG
GCGTCCAACTTTCTCAAACCGCTTTAGTTGCTGCAATTCGCCATGATTGTGCTGCATCAGGTATT
CATACCATTGCCACAGAACAATACAATCAAGATATTGTCAGTTTAGGTCTGCATGCCGCTCAAGA
TGTTTTAGAGATGGAGCAGAAATTACGCAATATTGTTTCAATGACAATTCTGGTAGCCTGTCAGG
CCATTCATCTTCGCGGCAATATTAGTGAAATTGCGCCTGAAACTGCTAAATTTTACCATGCAGTA
CGCGAAATCAGTTCTCCTTTGATCACTGATCGTGCGTTGGATGAAGATATAATCCGCATTGCGG
ATGCAATTATTAATGATCAACTTCCTCTGCCAGAAATCATGCTGGAAGAATAA

To create a vector capable of integrating the lacI-Ptac-mPAL1 sequence into the chromosome, Gibson assembly was used to add 1000 bp sequences of DNA homologous to the Nissle exo/cea locus to both sides of a flippase recombination target (FRT) site-flanked chloramphenicol resistance (cmR) cassette on a knock-in knock-out (KIKO) plasmid. Gibson assembly was then used to clone the lacI-Ptac-mPAL1 DNA sequence between these homology arms, adjacent to the FRT-cmR-FRT site. Successful insertion of the fragment was validated by sequencing. PCR was used to amplify the entire exo::FRT-cmR-FRT::lacI-Ptac-mPAL1::cea region. This knock-in PCR fragment was used to transform an electrocompetent Nissle strain that contains a temperature-sensitive plasmid, pKD46, which encodes the lambda red recombinase genes. After transformation, cells were grown for 2 hrs at 37° C. Growth at 37° C. cured the temperature-sensitive plasmid. Transformants with successful chromosomal integration of the fragment were selected on chloramphenicol at 30 μg/mL. These same methods may be used to create vectors capable of integrating the lacI-Ptac-mPAL1 sequence (SEQ ID NO: 507) at additional chromosomal integration sites. Amplified knock-in fragments from the different vectors were integrated in different strains and also integrated iteratively into the same strain, generating strains with multiple copies of mPAL1 on the chromosome.

The SYN7393 strain (lacI-malE/K::Ptac-mPAL1-mPAL1, rhtBC::lacI-Ptac-pheP) contains two copies of mPAL1 integrated at the malEK locus, with both genes operatively linked to a single IPTG-inducible Ptac promoter and co-transcribed in a bicistronic message. A copy of the transcriptional repressor, lacI, was included in the integration construct and divergently transcribed from both pheP and mPAL1-mPAL1 (FIG. 6B). The sequence of an exemplary construct is provided below in which the two copies of mPAL1 are co-transcribed under the control of an exemplary IPTG-inducible Ptac promoter. Nucleotide sequences in bold designate the IPTG inducible Ptac promoter, nucleotide sequences in italics designate either pheP or mPAL, underlined nucleotide sequences designate lacI and its constitutive promoter, nucleotide sequences in lowercase designate the second copy of mPAL in tandem, nucleotides in italics and underlined designate the ribosome binding site (RBS) directly upstream of the second copy of mPAL. SYN7393 also has a copy of pheP integrated at the rhtBC locus, operatively linked to a separate Ptac promoter.

Nucleotide sequence of mPALI-mPALI integration construct
(SEQ ID NO: 508)
TCACTGCCCGCTTTCCAGTCGGGAAACCTGTCGTGCCAGCTGCATTAATGAATCGGCCAA
CGCGCGGGGAGAGGCGGTTTGCGTATTGGGCGCCAGGGTGGTTTTTCTTTTCACCAGTGA
GACTGGCAACAGCTGATTGCCCTTCACCGCCTGGCCCTGAGAGAGTTGCAGCAAGCGGT
CCACGCTGGTTTGCCCCAGCAGGCGAAAATCCTGTTTGATGGTGGTTAACGGCGGGATAT
AACATGAGCTATCTTCGGTATCGTCGTATCCCACTACCGAGATATCCGCACCAACGCGCA
GCCCGGACTCGGTAATGGCGCGCATTGCGCCCAGCGCCATCTGATCGTTGGCAACCAGC
ATCGCAGTGGGAACGATGCCCTCATTCAGCATTTGCATGGTTTGTTGAAAACCGGACATG
GCACTCCAGTCGCCTTCCCGTTCCGCTATCGGCTGAATTTGATTGCGAGTGAGATATTTAT
GCCAGCCAGCCAGACGCAGACGCGCCGAGACAGAACTTAATGGGCCCGCTAACAGCGCG
ATTTGCTGGTGACCCAATGCGACCAGATGCTCCACGCCCAGTCGCGTACCGTCCTCATGG
GAGAAAATAATACTGTTGATGGGTGTCTGGTCAGAGACATCAAGAAATAACGCCGGAAC
ATTAGTGCAGGCAGCTTCCACAGCAATGGCATCCTGGTCATCCAGCGGATAGTTAATGAT
CAGCCCACTGACGCGTTGCGCGAGAAGATTGTGCACCGCCGCTTTACAGGCTTCGACGCC
GCTTCGTTCTACCATCGACACCACCACGCTGGCACCCAGTTGATCGGCGCGAGATTTAAT
CGCCGCGACAATTTGCGACGGCGCGTGCAGGGCCAGACTGGAGGTGGCAACGCCAATCA
GCAACGACTGTTTGCCCGCCAGTTGTTGTGCCACGCGGTTGGGAATGTAATTCAGCTCCG
CCATCGCCGCTTCCACTTTTTCCCGCGTTTTCGCAGAAACGTGGCTGGCCTGGTTCACCAC
GCGGGAAACGGTCTGATAAGAGACACCGGCATACTCTGCGACATCGTATAACGTTACTG
GTTTCATATTCACCACCCTGAATTGACTCTCTTCCGGGCGCTATCATGCCATACCGCGAA
AGGTTTTGCGCCATTCGATGGCGCGCCGCTTCGTCAGGCCACATAGCTTTCTTGTTCTGAT
CGGAACGATCGTTGGCTGTGTTGACAATTAATCATCGGCTCGTATAATGTGTGGAATT
GTGAGCGCTCACAATTAGCTGTCACCGGATGTGCTTTCCGGTCTGATGAGTCCGTG
AGGACGAAACAGCCTCTACAAATAATTTTGTTTAATAGCTTCGGTAAGTTTACGGAG
GATTTGTCATGAAAGCTAAAGATGTTCAGCCAACCATTATTATTAATAAAAATGGCCTTATCTCT
TTGGAAGATATCTATGACATTGCGATAAAACAAAAAAAAGTAGAAATATCAACGGAGATCACTGA
ACTTTTGACGCATGGTCGTGAAAAATTAGAGGAAAAATTAAATTCAGGAGAGGTTATATATGGAA
TCAATACAGGATTTGGAGGGAATGCCAATTTAGTTGTGCCATTTGAGAAAATCGCAGAGCATCA
GCAAAATCTGTTAACTTTTCTTGGCGCTGGTACTGGGGACTATATGTCCAAACCTTGTATTAAAG
CGTCACAATTTACTATGTTACTTTCTGTTTGCAAAGGTTGGTCTGCAACCAGACCAATTGTCGCT
CAAGCAATTGTTGATATGATTAATCATGACATTGTTCCTCTGGTTCCTCGCTATGGCTCAGTGGG
TGCAAGCGGTGATTTAATTCCTTTATCTTATATTGCACGAGCATTATGTGGTAAGGGCAAAGTTT
ATTATATGGGCGCAGAAATTGACGCTGCTGAAGCAATTAAACGTGCAGGGTTGACACCATTATC
GTTAAAAGCCAAAGAAGGTCTTGCTCTGATTAACGGCACCCGGGTAATGTCAGGAATCAGTGCA
ATCACCGTCATTAAACTGGAAAAACTATTTAAAGCCTCAATTTCTGCGATTGCCCTTGCTGTTGA
AGCATTACTTGCATCTCATGAACATTATGATGCCCGGATTCAACAAGTAAAAAATCATCCTGGTC
AAAACGCGGTGGCAAGTGCATTGCGTAATTTATTGGCAGGTTCAACGCAGGTTAATCTATTATCT
GGGGTTAAAGAACAAGCCAATAAAGCTTGTCGTCATCAAGAAATTACCCAACTAAATGATACCTT
ACAGGAAGTTTATTCAATTCGCTGTGCACCACAAGTATTAGGTATAGTGCCAGAATCTTTAGCTA
CCGCTCGGAAAATATTGGAACGGGAAGTTATCTCAGCTAATGATAATCCATTGATAGATCCAGA
AAATGGCGATGTTCTACACGGTGGAAATTTTATGGGGCAATATGTCGCCCGAACAATGGATGCA
TTAAAACTGGATATTGCTTTAATTGCCAATCATCTTCACGCCATTGTGGCTCTTATGATGGATAAC
CGTTTCTCTCGTGGATTACCTAATTCACTGAGTCCGACACCCGGCATGTATCAAGGTTTTAAAG
GCGTCCAACTTTCTCAAACCGCTTTAGTTGCTGCAATTCGCCATGATTGTGCTGCATCAGGTATT
CATACCATTGCCACAGAACAATACAATCAAGATATTGTCAGTTTAGGTCTGCATGCCGCTCAAGA
TGTTTTAGAGATGGAGCAGAAATTACGCAATATTGTTTCAATGACAATTCTGGTAGCCTGTCAGG
CCATTCATCTTCGCGGCAATATTAGTGAAATTGCGCCTGAAACTGCTAAATTTTACCATGCAGTA
CGCGAAATCAGTTCTCCTTTGATCACTGATCGTGCGTTGGATGAAGATATAATCCGCATTGCGG
ATGCAATTATTAATGATCAACTTCCTCTGCCAGAAATCATGCTGGAAGAATAAAACAACACCCAC
TAAGATAACTCTAGAAATAATTTTGTTTAACTTTAAGAAGGAGATATACATatgaaagctaaaga
tgttcagccaaccattattattaataaaaatggccttatctctttggaagatatctatgacattg
cgataaaacaaaaaaaagtagaaatatcaacggagatcactgaacttttgacgcatggtcgtgaa
aaattagaggaaaaattaaattcaggagaggttatatatggaatcaatacaggatttggagggaa
tgccaatttagttgtgccatttgagaaaatcgcagagcatcagcaaaatctgttaacttttcttg
gcgctggtactggggactatatgtccaaaccttgtattaaagcgtcacaatttactatgttactt
tctgtttgcaaaggttggtctgcaaccagaccaattgtcgctcaagcaattgttgatatgattaa
tcatgacattgttcctctggttcctcgctatggctcagtgggtgcaagcggtgatttaattcctt
tatcttatattgcacgagcattatgtggtaagggcaaagtttattatatgggcgcagaaattgac
gctgctgaagcaattaaacgtgcagggttgacaccattatgttaaaagccaaagaaggtcttgct
ctgattaacggcacccgggtaatgtcaggaatcagtgcaatcaccgtcattaaactggaaaaact
atttaaagcctcaatttctgcgattgcccttgctgttgaagcattacttgcatctcatgaacatt
atgatgcccggattcaacaagtaaaaaatcatcctggtcaaaacgcggtggcaagtgcattgcgt
aatttattggcaggttcaacgcaggttaatctattatctggggttaaagaacaagccaataaagc
ttgtcgtcatcaagaaattacccaactaaatgataccttacaggaagtttattcaattcgctgtg
caccacaagtattaggtatagtgccagaatctttagctaccgctcggaaaatattggaacgggaa
gttatctcagctaatgataatccattgatagatccagaaaatggcgatgttctacacggtggaaa
ttttatggggcaatatgtcgcccgaacaatggatgcattaaaactggatattgctttaattgcca
atcatcttcacgccattgtggctcttatgatggataaccgtttctctcgtggattacctaattca
ctgagtccgacacccggcatgtatcaaggttttaaaggcgtccaactttctcaaaccgctttagt
tgctgcaattcgccatgattgtgctgcatcaggtattcataccattgccacagaacaatacaatc
aagatattgtcagtttaggtctgcatgccgctcaagatgttttagagatggagcagaaattacgc
aatattgtttcaatgacaattctggtagcctgtcaggccattcatcttcgcggcaatattagtga
aattgcgcctgaaactgctaaattttaccatgcagtacgcgaaatcagttctcctttgatcactg
atcgtgcgttggatgaagatataatccgcattgcggatgcaattattaatgatcaacttcctctg
ccagaaatcatgctggaagaataa

To create a vector capable of integrating the lacI-Ptac-mPAL1-mPAL1 sequence (SEQ ID NO: 508) into the E. coli Nissle chromosome in SYN7393, the Gibson assembly was used to add 1000 bp sequences of DNA homologous to the Nissle exo and cea loci on either side of an FRT site-flanked kanamycin resistance (cmR) cassette on a KIKO plasmid. The integration construct was synthesized by Genewiz and Gibson assembly was then used to clone the lacI-Ptac-mPAL1-mPAL1 DNA sequence between these homology arms, adjacent to the FRT-cmR-FRT site. Successful insertion of the fragment was validated by sequencing. PCR was used to amplify the entire exo::FRT-camR-FRT::lacI-Ptac-mPAL1-mPAL1::cea region. This knock-in PCR fragment was used to transform an electrocompetent Nissle strain already containing lacI-Ptac-pheP in the rhtBC locus or lacI-Ptac-mPAL1 in the malEK locus as well as expressing the lambda red recombinase genes via plasmid pKD46. After transformation, cells were grown for 2 hrs at 37° C. Transformants with successful integration of the fragment were selected on chloramphenicol at 30 μg/mL. These same methods may be used to create a vector capable of integrating the lacI-Ptac-mPAL1-mPAL1 sequence (SEQ ID NO: 508) at additional chromosomal integration sites. Antibiotic resistance markers were removed using pCP20 transformation as described herein. More information on the construction and activity of SYNB1934 can be found in PCT/US2021/023003, PCT/US2021/063976, and U.S. Provisional Application No. 62/132,627, the disclosure of which are herein incorporated by reference.

SYNB1934v1 further comprises a deletion in the pks island (colibactin (clb) island) (see Genes: SEQ ID NOs: 294-312, and Polypeptides: SEQ ID NOs: 313-331).

### Example 3. Clinical Study of Safety, Tolerability, and Pharmacodynamics

In order to evaluate the safety and tolerability of SYNB1934 in human subjects, a study was performed in two parts, dose escalation and crossover study with SYNB1934 and SYNB1618, and a crossover study with SYNB1934 administered with or without protein pump inhibitor (PPI) supplementation.

Part 1: Dose escalation and crossover study with SYNB1934 and SYNB1618. Part 1 is a double-blind (sponsor-open), placebo-controlled, multiple-ascending dose (MAD) design, with a crossover component for a subset of subjects. The primary objective of Part 1 was to evaluate the safety and tolerability of SYNB1934 in human subjects. Secondary objectives were to evaluate the effects of SYNB1934 on D5-hippuric acid (D5-HA) amount excreted in urine over 6 hours after administration of a D5-phenylalanine (D5-Phe) tracer and compare those effects to those of SYNB1618, and the assess SYNB1934 microbial kinetics measured with qPCR following dosing. Exploratory objectives for the study included evaluation of the pharmacodynamic (PD) effects of SYNB1934 and SYNB1618 on plasma phenylalanine (Phe), plasma amino acids (in addition to Phe), plasma Phe metabolites, and urinary Phe metabolites, and evaluation the PD effects of SYNB1934 and SYNB1618 following oral administration of D5-Phe on plasma D5-Phe, plasma D5-Phe metabolites, and urinary D5-Phe metabolites.

Study cohorts 1 and 3 (and optionally 4 and 5) were randomly assigned according to a MAD design, with all subjects completing a treatment period with SYNB1934 or placebo, and additionally SYNB1618 (in Cohort 2 only), collectively referred to as “investigational medicinal product” (IMP). Subjects in Cohorts 1 and 3 (and, optionally, 4 and 5) participated in only one treatment period. Subjects in Cohort 2 underwent a ≥7-day washout period after the first treatment period, followed by a second (crossover) treatment period, in accordance with the following three treatment sequences:

SYNB1934 to SYNB1618: 6 subjects received SYNB1934 in the first treatment period and were crossed over to receive SYNB1618 (at the same live cell dose as SYNB1934) in the second treatment period.

SYNB1618 to SYNB1934: 6 subjects received SYNB1618 in the first treatment period and were crossed over to receive SYNB1934 (at the same live cell dose as SYNB1618) in the second treatment period.

Placebo to Placebo: 2 subjects received placebo in the first and second treatment periods.

For each treatment period, subjects reported to the clinical research unit (CRU) on Day −1 (or on Day −2, if preferred). On Day −1, baseline evaluations were performed, and subjects started on a controlled diet. A PPI (esomeprazole 40 mg) was initiated once daily (QD), 60-90 minutes before breakfast, starting 2 days before the first dose of IMP. A Tracer Study was also performed on Day −1. On the first day of IMP dosing, subjects were randomly assigned to treatment according to the MAD design. Subjects were dosed with IMP, immediately following meals, 3 times per day (TID) on Treatment Day 1 and once at breakfast on Treatment Day 2, for a total of 4 planned doses. To improve tolerability, a Dose-Ramp could be implemented for up to 4 days prior to Treatment Day 1. A second Tracer Study was performed on Treatment Day 2. Subjects were released from the CRU on Treatment Day 2 after completion of the Tracer Study and safety assessments. Crossover subjects (Cohort 2) underwent a ≥7-day washout (i.e., no administration of IMP or PPI) prior to re-entry to the CRU for the second treatment period; the first and second treatment period followed the same schedule of events. Subjects were followed in the study for at least 28 days after the last dose, or until two documented, negative fecal samples as analyzed by qPCR, whichever occurred later, or until completion of a course of antibiotics if a subject remains colonized 12 weeks following the last dose.

For each treatment period, subjects reported to the CRU on Day −1 (or on Day −2, if preferred). On Day −1, baseline evaluations were performed and a controlled diet was initiated. Subjects randomized to receive PPI initiated a PPI regimen (esomeprazole 40 mg QD, 60-90 minutes before breakfast) starting 2 days before the first dose of IMP. A Tracer Study was also performed on Day −1 for all subjects. Subjects were dosed with SYNB1934 immediately following meals TID on Treatment Day 1 and once at breakfast on Treatment Day 2, for a total of 4 doses planned for administration during the first treatment period. To improve tolerability, a Dose-Ramp could be implemented for up to 4 days prior to Treatment Day 1. A second Tracer Study was performed on Treatment Day 2. Subjects then underwent a ≥14-day washout (i.e., no administration of IMP or PPI), followed by initiation of a second treatment period in which they were crossed over to receive the alternate (i.e., PPI or no PPI) regimen. The first and second treatment period followed the same schedule of events. Subjects were released from the CRU on Treatment Day 2 of the second treatment period after completion of the Tracer Study and safety assessments. Subjects were followed in the study for at least 28 days after the last dose, or until two documented, negative fecal samples as analyzed by qPCR, whichever occurs later, or until completion of a course of antibiotics if a subject remains colonized 12 weeks following the last dose.

The primary endpoints for both Part 1 and Part 2 was the nature and frequency of treatment-emergent adverse events (TEAEs), and change from baseline in clinical laboratory assessments (e.g., blood chemistry, complete blood count [CBC], urinalysis), vital signs measurements, physical examinations, and electrocardiograms (ECGs), and plasma amino acids other than Phe. Secondary endpoints for the studies included change from baseline in urine D5-HA Aet over 6 hours after D5-Phe administration and following SYNB1934 dosing compared to: placebo (Part 1), SYNB1618 (Part 1 Cohort 2), and treatment with and without a PPI (Part 2), and SYNB1934 clearance, as measured in feces by qPCR, following dosing. Exploratory endpoints for the studies were change from baseline in plasma Phe and plasma trans-cinnamic acid (TCA) area under the curve (AUC) and urinary hippuric acid (HA) Aet over 6 hours during a Tracer Study following dosing with and without a PPI, and change from baseline in plasma D5-Phe and plasma D5-TCA AUC over 6 hours during a Tracer Study following dosing with and without a PPI.

Tracer Study: A Tracer Study was performed after an overnight fast (starting at 10 PM the previous day). After baseline blood and spot urine samples were collected, subjects received the tracer day diet (a meal replacement shake) followed by an oral dose of a D5-phenylalanine (D5-Phe) isotopic tracer at a dose of 1 g dissolved in 100 mL of diluent. The meal replacement shake, D5-Phe, and IMP (if applicable) were consumed over a 15-minute period. Blood and urine samples were taken at intervals for the following 6 hours. Subjects remained fasted (water only) until after the last tracer assessment was collected.

A Phe meal test was performed at baseline and following dosing with 2×10¹² live cell count SYNB1618 or SYNB1934 or placebo. After an overnight fast, subjects received a protein shake (20 g protein) and an oral dose of D5-Phe (1 g or 15 mg/kg). Blood and urine samples were collected for up to 24 hours. Plasma D5-Phe and its metabolites plasma D5-TCA and urine D5-HA were measured.

Optional Dose-Ramp: To improve tolerability, an optional Dose-Ramp could be implemented for up to 4 days prior to Treatment Day 1, where Treatment Day 1 was defined as the day on which full TID dosing was achieved. In the case of a Dose-Ramp, the Treatment Day 2 assessments (e.g., morning IMP administration, the second Tracer Study, and release from the CRU) would occur on the day after TID dosing of IMP was achieved on Treatment Day 1.

Follow-up Period: A subject's end of study (EOS) determination was dependent upon resolution or stabilization of adverse events (AEs) and was monitored weekly. Subjects who did not have an AE and had clearance of SYNB1934 and SYNB1618 were discharged from the study at the Safety Follow-up Visit (i.e., 28 days after discharge from the CRU, relative to the second treatment period for crossover subjects), which were performed either in the clinic or by telemedicine. Subjects who presented with AE(s) were followed until resolution or stabilization of the AE(s).

In addition, following completion of study dosing, subjects were followed to determine the clearance of SYNB1934 and SYNB1618 in the feces based on qPCR. Subjects were required to provide fecal samples for PCR assessment weekly for up to 12 weeks after the last dose of IMP until they had 2 consecutive negative fecal tests. Subjects who remained colonized after 12 weeks following the last dose were treated with a 3-day course of oral ciprofloxacin (or another antibiotic as per the Investigator's Brochure). An AE assessment was performed by telephone at least 7 days after completion of antibiotic treatment. If no AEs were reported, subjects were not required to do any further follow-up and were considered to have completed the study requirements. If AEs were reported, subjects were advised to return to the inpatient facility for a comprehensive physical examination, laboratory assessment, and AE causality determination.

Dose Cohorts and Dose Escalation: In Part 1, the starting dose of SYNB1934 was 3×10¹¹ live cells, based on clinical and nonclinical safety and tolerability of previously tested similar Escherichia coli Nissle (EcN)-based products. Dose-escalation decisions were made once at least 6 subjects in a cohort had been dosed and had at least 24 hours of post-dose observation. Dose escalation was up to approximately 3-fold per cohort, with the option to implement a Dose-Ramp of up to 4 days prior to Treatment Day 1 to improve tolerability. Decisions were made based on a blinded review of tolerability, clinical observations, safety laboratory assessments, and, optionally, PD. Of note, Part 1 dose-escalation decisions were made based on Part 1 MAD cohort safety and tolerability data from the dosing days and did not require any data that may have been available from the second treatment period for Cohort 2 (i.e., Part 1 Crossover) or Part 2.

The planned dose cohorts in Part 1 of the study are listed in the Table 9 below. Doses may be adjusted up or down based on ongoing assessments.

TABLE 9
Planned Dose Cohorts

| Part 1 Cohort | Number of Subjects | SYNB1934 Dose (# live cells) |
|---|---|---|
| 1 | 8 | 3 × 10¹¹ |
| | (6 treated: 2 placebo) | |
| 2 | 14 | 6 × 10¹¹** |
| | (12 treated*: 2 placebo) | |
| 3 | 8 | 1 × 10¹² |
| | (6 treated: 2 placebo) | |
| Optional 4 | 8 | TBD ≤ 2 × 10¹² |
| | (6 treated: 2 placebo) | |
| Optional 5 | 8 | TBD ≤ 2 × 10¹² |
| | (6 treated: 2 placebo) | |

*Active treatment comprises SYNB1934 and SYNB1618 in a crossover design.
**SYNB1618 will be administered at the same live cells dose as SYNB1934.

The MTD for Part 1 was defined as the dose immediately preceding the dose level at which >50% of subjects experienced an IMP-related National Cancer Institute (NCI) Common Terminology Criteria for Adverse Events (CTCAE)≥Grade 2 toxicity or ≥2 subjects experience a treatment-related≥Grade 3 toxicity.

### Inclusion Criteria:

1. Age≥18 years.
2. Able and willing to voluntarily complete the informed consent process.
3. Diagnosis of classic PKU based on medical history as assessed by the investigator (e.g., Phe concentration of >1200 μmol/L at any time, low dietary Phe tolerance, or genetic diagnosis).
4. Blood Phe≥600 μmol/L at Screening at current treatment regimen (diet and/or sapropterin at a stable dose).
5. Stable diet including stable medical formula regimen (if used) for at least 1 month prior to Screening.
6. Available for and agree to all study procedures, including urine and blood collection, adherence to diet control, follow-up visits, and IMP ingestion compliance.
7. Screening laboratory evaluations (e.g., chemistry panel, complete blood count with differential, urinalysis, creatinine clearance, C-reactive protein [CRP]) within normal limits or judged to be not clinically significant by the investigator.

### Exclusion Criteria:

1. Currently taking Palynziq® (pegvaliase-pqpz) (within 1 month of Screening).
Inflammatory bowel disease of any grade or irritable bowel syndrome requiring pharmacologic therapy.
3. History of or current immunodeficiency disorder.
4. Intolerance of or allergic reaction to E. coli Nissle or any of the ingredients in SYNB1618 or SYNB1934 formulation.
5. Any condition (e.g., celiac disease, gastrectomy, bypass surgery, ileostomy) or receiving prescription medication or over-the-counter product that may possibly affect absorption of medications or nutrients.
6. Currently taking or plans to take any type of systemic (e.g., oral or intravenous) antibiotic within 28 days prior to the first anticipated dose of IMP through final safety assessment, including planned surgery, hospitalizations, dental procedures, or interventional studies that are expected to require antibiotics. Exception: topical antibiotics are allowed.
7. Within 3 months prior to anticipated first dose, major surgery (an operation upon an organ within the cranium, chest, abdomen, or pelvic cavity) or inpatient hospital stay.
8. Dependence on alcohol or drugs of abuse.
9. Administration or ingestion of an investigational drug within the 30 days or 5 half-lives before Screening, whichever is longer, or cell/gene therapy prior to the Screening visit, or current enrollment in an investigational drug study.
10. Acute or chronic medical, surgical, psychiatric, or social condition or laboratory abnormality that may increase patient risk associated with study participation, compromise adherence to study procedures and requirements, or may confound interpretation of study safety or PD results and, in the judgment of the investigator, would make the patient inappropriate for enrollment.

### Example 4. Statistical Methods

### Baseline

Baseline is the last scheduled measurement prior to the first IMP administration, unless specifically described below. If a sequence of baseline measurements is taken predose on the same day, time-matched baseline is used. If multiple measurements are included within a baseline measurement (e.g., repeated measures at the same nominal time), the arithmetic mean of the multiple samples is considered the baseline. If a parameter is calculated (e.g., AUC), the final predose calculated value is considered the baseline.

For all assessments, baseline is in the same period. If baseline is unavailable for the current period, baseline is imputed, if required and available, from a separate period with the same assessments. Baseline imputation is not performed for statistical analyses (e.g., mixed-model with repeated measures).

### Populations for Analysis

The following populations are defined:
Efficacy/PD: all subjects in the safety population who have at least one baseline and post-baseline PD measurement and have not missed >50% of the IMP doses during the dosing period or any dose on the day of the Tracer Study
Per protocol: all subjects in the safety population who complete the treatment period and do not have any major protocol deviations
Safety: all subjects who receive at least one dose of IMP

### Efficacy Analysis

The secondary endpoint of D5-HA Ae change from baseline is analyzed on the log scale by a mixed model with repeated measures with fixed effects for treatment, time (baseline or on-treatment), treatment by time, and (if applicable) period and a random effect by subject. Reporting converts the log-scale estimate of change from baseline and change from baseline, change from placebo to the percent scale.

The model is estimated separately for:
Part 1, SYNB1934 or placebo excluding Part 1, Cohort 2, Period 2
Part 1, Cohort 2
Part 2

### Pharmacodynamic Analysis

Urine, blood, and fecal samples are collected during the Screening Period and on study both before and after administration of IMP. The following laboratory measurements are performed to evaluate the preliminary PD of SYNB1934 and SYNB1618:
Plasma Phe and D5-Phe
Plasma amino acids (in addition to Phe)
Plasma Phe and D5-Phe metabolites
Urinary Phe and D5-Phe metabolites

AUC is calculated for plasma Phe and D5-Phe and their metabolites, and Aet is calculated for urine Phe and D5-Phe metabolites.

### Example 5. Clinical Efficacy and Safety in Patients with Phenylketonuria

A study was performed to evaluate the efficacy and safety of SYNB1618 in patients with phenylketonuria. The primary objective of the study was to determine the efficacy of SYNB1618 in reducing area under the curve (AUC) for plasma D5-phe. Secondary objectives of the study were to determine the efficacy of SYNB1618 in reducing plasma Phe, and to evaluate the safety and tolerability of SYNB1618. Exploratory objectives of the study were to evaluate the effect of SYNB1618 on PD biomarkers of Phe and Phe metabolites, including TCA and HA, and to evaluate the effect of SYNB1618 on cognitive function using the Cambridge Neuropsychological Test Automated Battery (CANTAB).

The study was a 2-arm, open-label study of a dose-ramp regimen consisting of 4 dose levels of SYNB1618 (IMP) (1×10¹¹, 3×10¹¹, 1×10¹¹, and 2×10¹¹ live cells) over 15 days of treatment (Arm 1). All patient evaluations and assessments throughout this study were conducted either at the clinical site or by a home healthcare professional at an alternative location (e.g., patient's home, hotel).

Patients were screened for eligibility within 52 days prior to enrollment. On Days −7 to −2, patients participated in a Diet Run-in Period. During the Diet Run-in Period, patients began taking a PPI once per day (QD), 60 to 90 minutes before a meal of their choice. Administration of PPI continued through the last day of dosing. Daily diet recording continued through the Follow-up Visit.

In Arm 1, oral administration of SYNB1618 was as follows:

On Day 1, patients took SYNB1618, 1×10¹¹ live cells twice per day (BID), and on Days 2 and 3, patients took SYNB1618, 1×10¹¹ live cells 3 times per day (TID) immediately after meals. On Days 4 to 6, SYNB1618 were escalated to a dose of 3×10¹¹ live cells TID immediately after meals. On Days 7 to 13, the dose of SYNB1618 were escalated to 1×10¹¹ live cells TID immediately after meals. On Days 14 and 15, patients will receive 2×10¹² live cells QD.

On Days 1 to 3, patients took the recommended dose QD (after lunch). On Days 4 to 6, patients took the recommended dose BID (after breakfast and dinner). On Days 7 to 13, patients took the recommended dose TID (immediately after meals). On Days 14 and 15, the dose did exceed 2×10¹² live cells QD.

Any patient permanently discontinuing IMP underwent a 2-week washout period and safety assessments; these patients were not required to remain on the protocol-mandated diet after discontinuation of IMP. At the Follow-up Visit, blood samples were collected to measure plasma Phe in order to assess the reversibility of Phe lowering. Patients continued to report unsolicited adverse events through 28 days after the last dose of IMP (i.e., End of Study).

Patients were also required to maintain a diet diary from Day−7 through Day 29 (Follow-up Visit) and an IMP administration and adverse event diary from Day −1 through Day 29 (Follow-up Visit).

Dietary Regimen: During the Screening Period, patients recorded their diet for at least 5 days using a daily food intake log and will be interviewed by a dietician. The dietician used interview results and the food intake record to create customized cycle menus listing foods and serving sizes for meals. Menus were designed to maintain calories, protein and Phe intake consistent with the patient's baseline diet.

Patients followed these menus and recorded their daily diet intake from Diet Run-in Period starting Day −7 through the Follow-up Visit on Day 29. The dietician or site staff member contacted the patient approximately 3 times each week to encourage compliance.

Tracer Study: The tracer study was conducted at Baseline (Day −1) and on Day 14. Patients fasted overnight (starting at 10 PM the previous day), after which fasting blood and spot urine samples were collected. Patients then received a meal supplement shake followed by an oral dose of a stable isotope of Phe (D5-Phe; tracer) at a dose of 1 g dissolved in 100 mL of diluent, followed by IMP (on Day 14 only). The meal supplement shake, Phe isotope, and IMP were consumed over a 15-minute period. Blood and urine samples for the Phe tracer were taken at intervals for the following 24 hours. Patients remained fasting (water only) until after the 6-hour tracer assessment was collected. Patients could then eat lunch after which remaining blood and urine samples were collected.

Biomarker Study: A biomarker study was conducted on Days 1, 7, and 15. Patients fasted overnight (starting at 10 PM the previous day), after which fasting blood and spot urine samples were collected. Patients then received a low/minimal Phe breakfast, followed by IMP administration (only on Days 7 and 15). Blood and urine samples were collected at intervals over the next 4 hours to assess plasma Phe and TCA and urine HA. Patients could then eat lunch.

The following endpoints were assessed for both treatment arms: 1) changes from baseline in labeled Phe (D5-Phe) in plasma, as measured by D5-Phe AUC over 24 hours after D5-Phe administration, on Day 14, at SYNB1618 dose of 2×10¹² live cells in Arm 1 or at the SYNB1934 highest dose in Arm 2, 2) changes from baseline in fasting levels of plasma Phe on Days 7, 14, and at the Follow-up Visit (Day 29±3), 3) changes from baseline in plasma TCA after a low/minimal Phe meal, as measured by TCA AUC over 4 hours on Days 7 and 15, 4) changes from baseline in urine HA on Days 7 and 15, 5) treatment-emergent adverse events (TEAEs), clinical laboratory parameters (including tyrosine and CRP), and vital signs, and 6) changes in CANTAB item scores from baseline to Day 14.

### Example 6. Interim Results

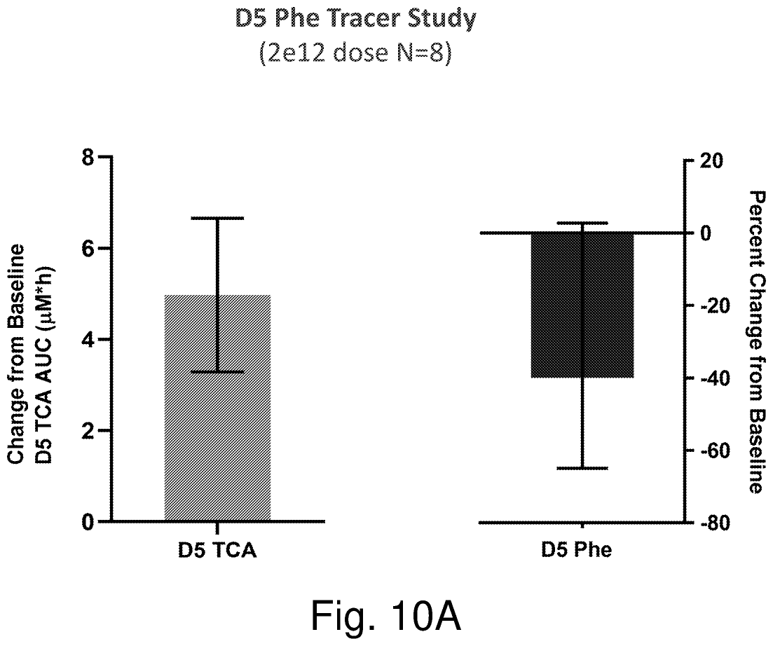

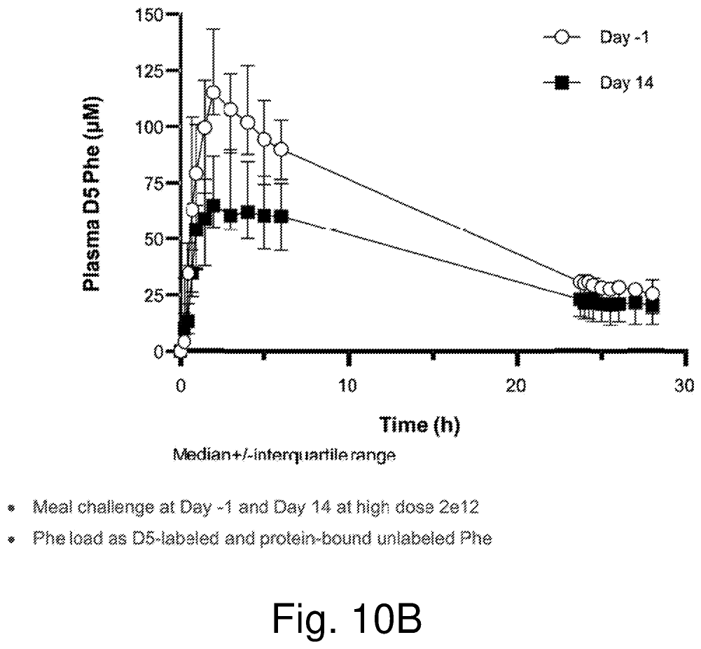

This Example discusses interim results of the clinical study with 8 PKU patients. Topline results and final analysis with 11 PKU patients are presented in Example 9. Administration of SYNB1618 at a 2×10¹² dose lowered the amount of D5-Phe and increased the amount of D5-TCA found in blood. FIG. 10A depicts the mean percent change of D5-Phe in blood at Day 14 compared to baseline in PKU patients. FIG. 10A also depicts the mean change in D5-TCA production in blood at Day 14 compared to baseline in PKU patients. FIG. 10B depicts the reduction in plasma D5-Phe over time at Day 14 compared to baseline in PKU patients after undergoing a meal challenge (protein shake (20 g) and D5-Phe (1 g)). Of the eight PKU patients administered SYNB1618 at a 2×10¹² dose, four experienced a greater than 40% reduction in plasma D5-Phe after the meal challenge on Day 14. These results show that the genetically engineered bacteria reduce the amount of D5-Phe in blood and increase production of D5-TCA, which indicates that the genetically engineered bacteria are active in the GI tract and actively consume phenylalanine.

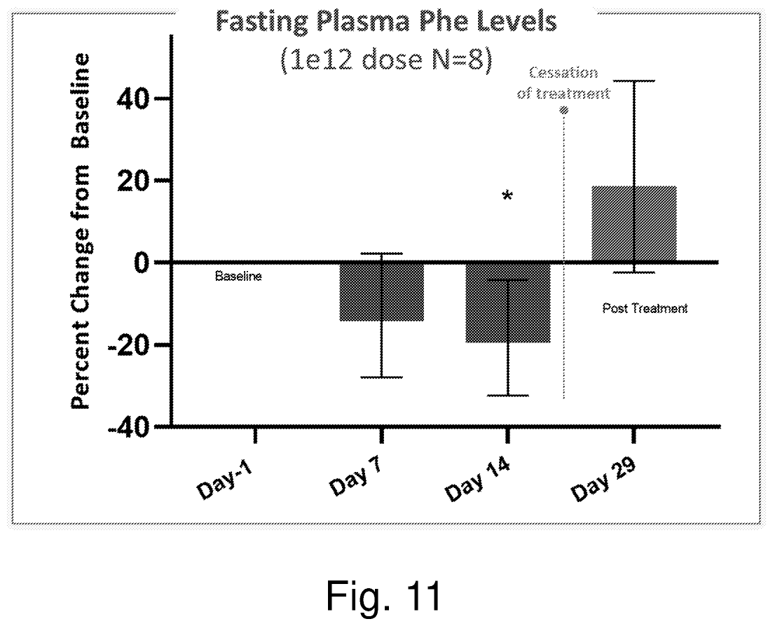

After administration with SYNB1618, FIG. 11 shows a rapid reduction in blood phenylalanine levels in PKU patients at Day 7 (3×10¹¹ dose) as a percent change compared to baseline, with a mean 21% reduction in blood phenylalanine levels at Day 14 (1×10¹² dose). Four out of the eight PKU patients in the study experienced greater than 20% reduction in blood phenylalanine levels at either the 3×10¹¹ or 1×10¹² dose levels.

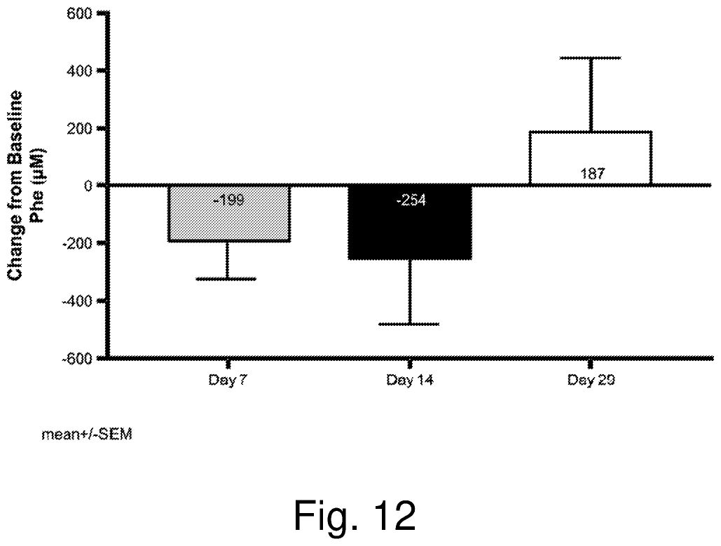

PKU patients with >20% reduction in the amount of Phe (e.g., μM plasma Phe) compared to baseline at Day 7 or Day 14 were considered responders. FIG. 12. shows the absolute reduction in the amount of Phe (e.g., μM plasma Phe) for patients (n=8) on SYNB1618 compared to baseline at Day 7 or Day 14. The mean reduction in Phe was 254 μM in the responder population (n=4).

Interim data from the 9 PKU subjects who completed dosing showed a least-squares mean Phe reduction at the 1×10¹² live cell dose that was −20.1% (95% CI: −31.3, −7.2). An increase in blood Phe levels was observed 2 weeks after the last dose, while subjects were still following the tightly controlled study diet (least-squares mean: 14.3%; 95% CI: −3.8, 35.7). Five subjects had at least 20% Phe lowering at either dose level. In four subjects, the fasting blood Phe level fell below 600 μmol/L. On Day 7 after the dose-ramp period, the change in blood Phe ranged from +18.4% to −34.9%, and on Day 14 at the 1×10¹² dose, Phe ranged from +14.4% to −59.9%. The mean (standard error of the mean) absolute change in Phe in responders was 186 (49.8) μmol/L on Day 7 and −237 (89.4) μmol/L on Day 14.

As part of the interim analysis, the activity of SYNB1618 in PKU subjects was further evaluated by the D5-Phe tracer meal challenge. After an overnight fast, subjects were given a protein shake with 20 g of protein and 1 g of D5-Phe tracer followed by SYNB1618 (or nothing at baseline). Everything was consumed within 15 minutes. Blood and urine samples were collected for 24 hours. Labeled and unlabeled Phe and TCA in plasma and the amount of labeled and unlabeled HA excreted in urine were measured. A least-squares mean (95% CI) reduction in plasma D5-Phe AUC of −37.9 (−60.7, −1.7) was observed.

At the time of the interim analysis, initial clinical studies of SYNB1618 resulted in a least-squares mean reduction in D5-Plasma Phe AUC of −37.9% (95% CI, −60.7 to 1.7) and a corresponding plasma D5-TCA and urinary D5-HA biomarker signal (n=9 PKU subjects). Reduction in fasting blood Phe was also observed (mean, 14.5%; 95% CI, −26.5, −0.4) at the 3×10¹¹ dose and at the 1×10¹² live cell dose (least-squares mean, −20.1%; 95% CI, −31.3, −7.2). Reduction in D5-Phe was observed compared to baseline at the 2×10¹² live cell dose (least-squares mean, −40%; 90% CI, −65 to 3). No Serious Adverse Events (SAEs) or systemic safety issues identified. The tolerability profile in Phase 2 interim analysis was consistent with the experience in healthy volunteers. Most adverse events were mild-to-moderate and GI in nature. One discontinuation in Phase 2 interim analysis, which was determined to be not study related (anxiety due to PKU).

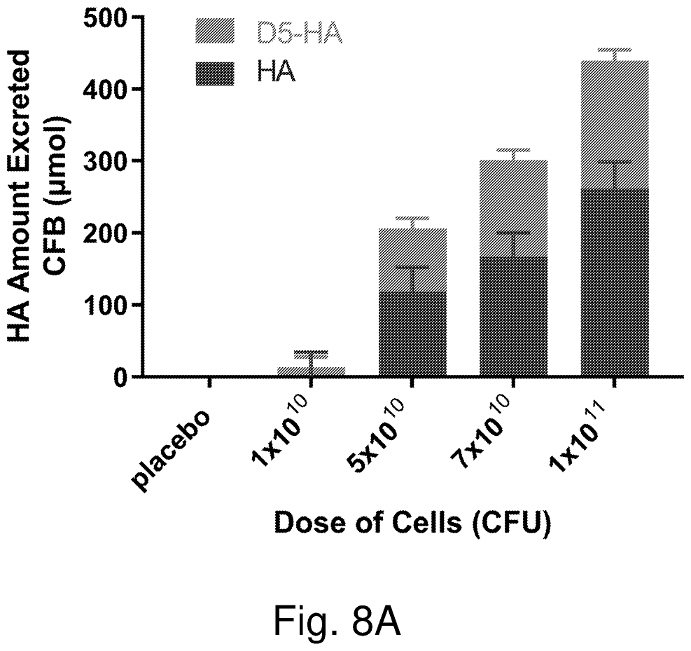

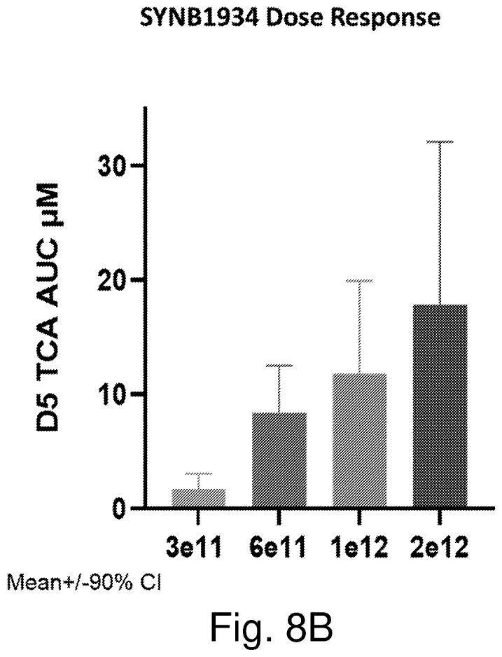

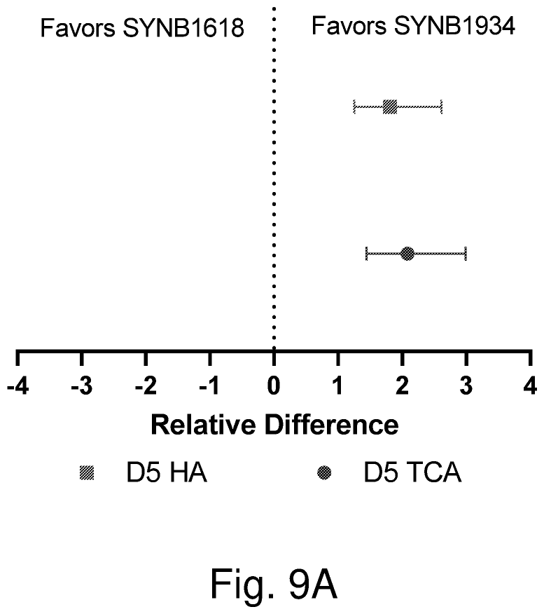

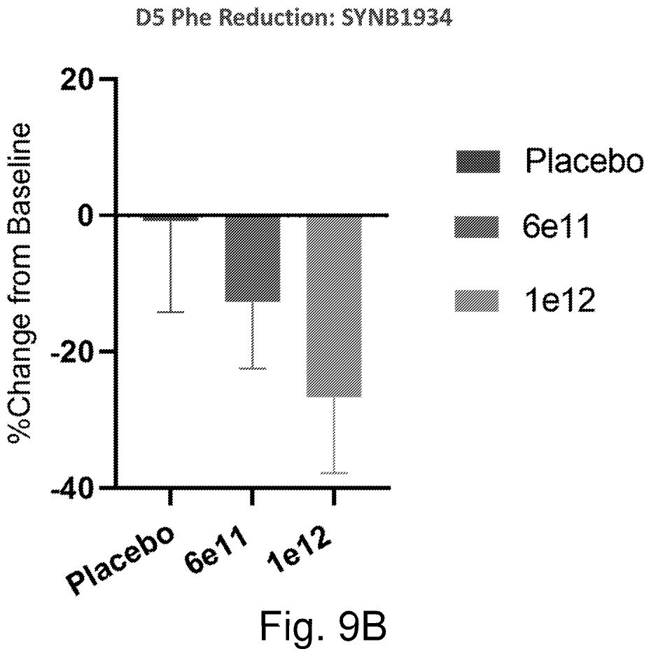

FIG. 8A depicts the effect administering SYNB1618 has on D5-HA production in healthy volunteers. strain-specific biomarkers (HA, D5-HA) were used to confirm dose-response in healthy volunteers. The study confirmed consistency in activity (with the same biomarkers) for healthy volunteers and PKU patients (n=10). TEAEs were generally mild-moderate in nature; predominantly GI-related AEs and headache. There were no treatment related SEAs and there was no systemic toxicity. No fecal sample was above the limit of quantification for SYNB1618 at 4 days after the last dose. The maximum tolerated dose (MTD) was reached at 1×10¹² for SYNB1618. To convert CFU to live cells, a conversion factor of 0.367 can be applied. For example, 1e10 CFU corresponds to 2.73e10 live cells in this study.

FIG. 8B depicts the dose-dependent effect administering SYNB1934 has on D5-TCA production in healthy volunteers. After three doses on Day 1 and one dose on Day 2, mean change in D5-TCA production was about 2, 8, and 12 for 3×10¹¹, 6×10¹¹, or 1×10¹² dose levels, respectively. TEAEs were generally mild-moderate in nature. No SAEs or systemic toxicity observed for either SYNB1618 or SYNB1934. At the highest dose (2×10¹²), the protocol-defined criteria for a maximum tolerated dose (MTD) was not reached for SYNB1934. Similar increases were observed in plasma D5-TCA. No subject had a positive fecal qPCR result 2 weeks after the last dose of IMP.

### Example 7. Statistical Methods

All patients are assigned to the SYNB1618 or SYNB1934 dose-ramp regimen. Patients who complete Arm 1 may enroll into Arm 2 and thereby receive both SYNB1618 and SYNB1934. Baseline is defined as the last scheduled measurement before first IMP administration, unless explicitly specified for an endpoint below. If a sequence of baseline measurements is taken predose on the same day, time-matched baseline is used. If multiple measurements are included within a baseline measurement (e.g., repeated measures at the same nominal time), the arithmetic mean of the multiple samples is considered the baseline. If a parameter is calculated (e.g., AUC), the final predose calculated value is considered the baseline. For clarity, D5-Phe AUC₀₋ₗₐₛₜ also follows these rules.

### Populations for Analysis

The following populations are defined:
Efficacy/pharmacodynamic: all patients in the safety population who have at least one post-baseline pharmacodynamics measurement.
Per protocol: all patients who complete the Treatment Period through the final tracer sample and do not have any major protocol deviations. Patients who require oral antibiotics during the course of the study should continue their dosing regimen but are not included in the per-protocol population.
Safety/modified intent to treat: all patients who receive at least 1 dose of IMP.

### Efficacy Analysis

Change from baseline in D5-Phe AUC0-last are analyzed for each arm by an analysis of covariance with covariates of baseline and treatment.

Fasting phenylalanine concentrations are analyzed by a mixed-model with repeated measures with categorical fixed effects for treatment by time and continuous baseline Phe and a random effect for patient (estimates are performed on the log scale and back-transformed to ratios for reporting). The effect size is compared to baseline using appropriate estimates from the statistical model.

### Sample Size

The study is powered to detect a 20% change in D5-Phe AUC₀₋ₗₐₛₜ lowering at a dose of SYNB1618 2×10¹² live cells in Arm 1 and the highest dose of SYNB1934 in Arm 2. This 20% decrease is −0.22 on the log scale. With the log-scale standard deviation for D5-Phe AUC₀₋ₗₐₛₜ of 0.20 estimated from patients with PKU in the multiple-dose part of Study SYNB1618-CP-001, a 2-sided t-test with 11 patients completing the study in the per-protocol population at a dose of 2×10¹² live cells has >90% power with 5% significance in each treatment arm.

After at least 6 patients have completed an arm, analyses of available data may be performed to determine if the arm or study may be stopped early for either efficacy or futility, based on the observed effect size and variability. Stopping an arm at an interim analysis does not imply that the study or the other arm is stopped.

### Example 8. Dietary Management

Clinical trials targeted at treating patients with phenylketonuria (PKU) require stability in dietary phenylalanine (Phe) intake for accurate interpretation of drug effect. Traditionally patients are educated in maintaining a stable diet and track Phe intake by using 3-day diet records which has been linked to poor compliance and inaccuracy. Dietary management during SynPheny-1 included a novel approach to decrease diet variability by using set menus with frequent dietitian involvement. Interim data of 8 patients' experience is presented.

For 36 days subjects adhered to a set of 7 customized, daily menus developed by metabolic dietitians based on their usual dietary intake according to 5-day food records and dietitian interviews. The menus followed the patient's baseline Phe intake±10% and were accessible via a mobile app or paper copies which allowed the subject to choose a menu to follow each day and record adherence. Study dietitians customized menus, educated the patient on menu adherence, accessed daily diet records in real-time, problem-solved menu deviations, and contacted the patient up to 3 times weekly to support dietary compliance.

4/8 patients used the app to access and log menu selection while 4/8 used the paper version. Total baseline dietary Phe deviations were <10% for 5/8 patients during diet run-in and 1/8 during dosing. Baseline deviations were <25% for 8/8 patients during diet run-in, 3/8 during dosing, and 4/8 during follow-up. Of the 36 study days, the median number of baseline deviations was 6.5 days (range 1-24 days) and the median percent deviation from baseline on those days was 18% (range 12-36%). 7/8 subjects had <10 baseline Phe deviations total during the study. Patients reported satisfaction with dietary support during the trial but most found using the menus to be monotonous. All study dietitians reported satisfaction with real-time data reporting from app output.

This new method resulted in tight baseline dietary Phe control during diet initiation and termination. Real-time compliance monitoring was possible with dietary support. Overall, this method was useful to control baseline Phe intake during a small Phase 2 clinical trial.

### Example 9. Clinical Efficacy and Safety in Patients with Phenylketonuria Treated with SYNB1934

A study was performed to evaluate the efficacy and safety of SYNB1934 in patients with phenylketonuria according to the methods described above for SYNB1618, e.g., in Example 5.

Adult patients (aged≥18 years) with PKU and Phe≥600 μmol/L at screening were eligible for enrollment if they had a stable diet (stable prescription for medical formula, stable allowance for Phe containing protein and stable adherence with this plan) for at least one month prior to screening. Patients were permitted to continue sapropterin dihydrochloride dosing if stable, but Pegvaliase use was an exclusion criterion. The study excluded patients with conditions that could affect absorption of medications or nutrients. Antibiotics were prohibited within 28 days prior to the first anticipated dose of investigational medicinal product (IMP) and through the final safety assessment.

This study was initially designed to evaluate the safety and efficacy of SYNB1618 (Arm 1) in patients with PKU but subsequently was modified to include the derived strain SYNB1934 (Arm 2). Both strains were evaluated in a multicenter, 2-arm, open-label trial to investigate the efficacy and safety of orally administered SYNB1618 or SYNB1934 in PKU patients using a dose-ramp regimen consisting of up to 4 dose levels given up to 3 times daily (TID) over 15 days (Table 10).

TABLE 10
Dose ramping regimen of IMP in SYNB1618-CP-003

| Study Days | Oral Dose of IMP (live cells) |
|---|---|
| Arm 1 | SYNB1618 |
| 1 | 1 × 10¹¹ BID (after lunch and dinner) |
| 2 and 3 | 1 × 10¹¹ TID (immediately after meals) |
| 4, 5, and 6 | 3 × 10¹¹ TID (immediately after meals) |
| 7, 8, 9, 10, 11, 12, and 13 | 1 × 10¹² TID (immediately after meals) |
| 14 and 15 | 2 × 10¹² QD (after breakfast) |
| Arm 2 | SYNB1934 |
| 1 | 3 × 10¹¹ BID (after lunch and dinner) |
| 2 and 3 | 3 × 10¹¹ TID (immediately after meals) |
| 4, 5, and 6 | 6 × 10¹¹ BID (immediately after meals) |
| 7, 8, 9, 10, 11, 12, and 13 | 1 × 10¹² TID (immediately after meals) |
| 14 and 15 | 2 × 10¹² QD (after breakfast) |

The primary trial endpoint was the change from baseline in plasma D5-Phe on Day 14 compared to Day −1, as measured by D5-Phe area under the curve (AUC) over 24 hours after D5-Phe administration. The secondary efficacy endpoint was a change from baseline in fasting plasma Phe on Days 7, 14, and at the follow-up visit (Day 29±3). Exploratory endpoints included change from baseline in plasma D5-TCA_{AUC} and urine D5-HA amount excreted (Aet) after D5-Phe administration on Day 14; change from baseline in plasma TCA and urine HA Aet on Days 7 and 15 (biomarker study); and changes in selected Cambridge Neuropsychological Test Automated Battery (CANTAB) item scores from baseline to Day 14.

Safety assessments included the frequency, type, and severity of treatment-emergent adverse events (TEAEs) and changes in clinical laboratory parameters and vital signs. Adverse events were monitored from Day −7 through Day 29 follow-up and were coded using the Medical Dictionary for Regulatory Activities, version 24.0. The severity of TEAEs and laboratory abnormalities were graded using the National Cancer Institute Common Terminology Criteria for Adverse Events, version 5.0.

The study was an open-label study of a dose-ramp regimen consisting of up to 4 dose levels of SYNB1934 (IMP) at a well-tolerated dose up to 2×10¹² as determined from the healthy volunteer study. Investigational product, dosage, and mode of administration was SYNB1934 administered orally at 3×10¹¹ live cells BID or TID, 6×10¹¹ live cells TID, 1×10¹² live cells TID, and 2×10¹² live cells QD. All patient evaluations and assessments throughout this study were conducted either at the clinical site or by a home healthcare professional at an alternative location (e.g., patient's home, hotel).

Patients were screened for eligibility within 52 days prior to enrollment. Diet was managed using personalized food menus designed to maintain calories, protein, and Phe intake consistent with each patient's baseline diet. On Days −7 to −2, patients participated in a Diet Run-in Period. During the Diet Run-in Period, patients began taking a PPI (PPI; esomeprazole 40 mg QD) once per day (QD), 60 to 90 minutes before a meal of their choice. Administration of PPI continued through the last day of dosing. Daily diet recording continued through the Follow-up Visit.

The oral administration dose of SYNB1934 was based on safety and tolerability observations from the healthy volunteer study described herein. A SYNB1934 maximum dose of 2×10¹² live cells was administered during the instant study. The 15-day schedule was as follows:

On Day 1, patients received SYNB1934, 3×10¹¹ live cells twice per day (BID), and on Days 2 and 3, patients took SYNB1934, 3×10¹¹ live cells 3 times per day (TID) immediately after meals. On Days 4 to 6, SYNB1934 was escalated to a dose of 6×10¹¹ live cells TID immediately after meals. On Days 7 to 13, the dose of SYNB1934 was escalated to 1×10¹² live cells TID immediately after meals. On Days 14 and 15, the dose of SYNB1934 was 2×10¹² live cells once daily (QD) immediately after meals.

Any patient permanently discontinuing IMP underwent safety assessments within 2 weeks of the last dose; these patients were not required to remain on the protocol-mandated diet after discontinuation of IMP. At the Follow-up Visit, blood samples were collected to measure plasma Phe in order to assess the reversibility of Phe lowering. The follow up period was 28 days.

Patients continued to report unsolicited adverse events through 28 days after the last dose of IMP (i.e., End of Study). Patients were also required to maintain a diet diary from Day−7 through Day 29 (Follow-up Visit) and an IMP administration and adverse event diary from Day −1 through Day 29 (Follow-up Visit).

Dietary Regimen: During the Screening Period, patients recorded their diet for at least 5 days using a daily food intake log and will be interviewed by a dietitian. The dietitian used interview results and the food intake record to create customized cycle menus listing foods and serving sizes for meals. Menus were designed to maintain calories, protein and Phe intake consistent with the patient's baseline diet.

Patients followed these menus and recorded their daily diet intake from Diet Run-in Period starting Day −7 through the Follow-up Visit on Day 29. The dietitian or site staff member contacted the patient approximately 3 times each week to encourage compliance.

Tracer Study: The tracer study was conducted at Baseline (Day −1) and on Day 14. Patients fasted overnight (starting at 10 PM the previous day), after which fasting blood and spot urine samples were collected. Patients then received a meal supplement shake followed by an oral dose of a stable isotope of Phe (D5-Phe; tracer) at a dose of 1 g dissolved in 100 mL of diluent, followed by IMP (on Day 14 only). The meal supplement shake, Phe isotope, and IMP were consumed over a 15-minute period. Blood and urine samples for the Phe tracer were taken at intervals for the following 24 hours. Patients remained fasting (water only) until after the 6-hour tracer assessment was collected. Patients could then eat lunch after which remaining blood and urine samples were collected.

Biomarker Study: A biomarker study was conducted on Days 1, 7, and 15. Patients fasted overnight (starting at 10 PM the previous day), after which fasting blood and spot urine samples were collected. Patients then received a low/minimal Phe breakfast, followed by IMP administration (only on Days 7 and 15). Blood and urine samples were collected at intervals over the next 4 hours to assess plasma Phe and TCA and urine HA. Patients could then eat lunch.

The following endpoints were assessed for both treatment arms: 1) changes from baseline in labeled Phe (D5-Phe) in plasma, as measured by D5-Phe AUC over 24 hours after D5-Phe administration, on Day 14, at SYNB1934 dose of 2×10¹² live, 2) changes from baseline in fasting levels of plasma Phe on Days 7, 14, and at the Follow-up Visit (Day 29±3), 3) changes from baseline in plasma TCA after a low/minimal Phe meal, as measured by TCA AUC over 4 hours on Days 7 and 15, 4) changes from baseline in urine HA on Days 7 and 15, 5) treatment-emergent adverse events (TEAEs), clinical laboratory parameters (including tyrosine and CRP), and vital signs, and 6) changes in CANTAB item scores from baseline to Day 14.

### Inclusion Criteria:

1. Age≥18 years.
2. Able and willing to voluntarily complete the informed consent process.
3. Diagnosis of classic PKU based on medical history as assessed by the investigator (e.g., Phe concentration of >1200 μmol/L at any time, low dietary Phe tolerance, or genetic diagnosis).
4. Blood Phe≥600 μmol/L at Screening.
5. Stable diet including stable medical formula regimen (if used) for at least 1 month prior to Screening.
6. Available for and agree to all study procedures, including urine and blood collection, adherence to diet control, follow-up visits, and IMP ingestion compliance.
7. Screening laboratory evaluations (e.g., chemistry panel, complete blood count [CBC] with differential, urinalysis, creatinine clearance, CRP) within normal limits or judged to be not clinically significant by the investigator.

### Exclusion Criteria:

1. Currently taking Palynziq® (pegvaliase-pqpz) (within 1 month of Screening).
2. Inflammatory or irritable bowel disorder of any grade.
3. History of or current immunodeficiency disorder.
4. Intolerance of or allergic reaction to E. coli Nissle or any of the ingredients in SYNB1934 formulation.
5. Any condition (e.g., celiac disease, gastrectomy, bypass surgery, ileostomy) or receiving prescription medication or an over-the-counter product that may possibly affect absorption of medications or nutrients.
6. Currently taking or plans to take any type of systemic (e.g., oral or intravenous) antibiotic within 28 days prior to the first dose of IMP through final safety assessment, including planned surgery, hospitalizations, dental procedures, or interventional studies that are expected to require antibiotics. Exception: topical antibiotics are allowed.
7. Within the 3 months prior to anticipated first dose, major surgery (an operation upon an organ within the cranium, chest, abdomen, or pelvic cavity) or inpatient hospital stay.
8. Dependence on alcohol or drugs of abuse.
9. Administration or ingestion of an investigational drug within the 30 days or 5 half-lives before Screening, whichever is longer, or cell/gene therapy prior to the Screening visit, or current enrollment in an investigational drug study.
10. Acute or chronic medical, surgical, psychiatric, or social condition or laboratory abnormality that may increase patient risk associated with study participation, compromise adherence to study procedures and requirements, or may confound interpretation of study safety or PD results and, in the judgment of the investigator, would make the patient inappropriate for enrollment.

### Results

The Phase 2 Synpheny-1 study was a Phase 2, open-label, 28-day study to assess both SYNB1618 and SYNB1934 in patients with PKU. The study included a dose-ramp regimen over 15 days of treatment, with days 7 through 14 at the constant dose of 1×10¹² live cells. The primary endpoint is the change in area under the curve (AUC) of plasma levels of labeled D5-phenylalanine (D5-Phe) after a meal challenge before and after the treatment period, a specific indicator of each drug candidate's ability to consume Phe as intended. Additional endpoints include change from baseline in fasting levels of plasma Phe, and incidence of treatment-emergent adverse events (TEAEs), as well as the levels of additional strain-specific metabolites plasma D5-TCA and urinary D5-HA. Dietary intake of Phe was carefully managed during the study to match patients' usual protein and Phe intake.

Synpheny-1 enrolled 20 adults with PKU who had a Phe level above 600 mol/L at screening despite treatment with diet and/or sapropterin. 11 patients received SYNB1618 and 9 received SYNB1934. 10 patients have completed the SYNB1618 arm. Study dosing was completed for 15 of 20 patients (10 patients in Arm 1 and 5 patients in Arm 2). One of 11 participants (9.1%) in Arm 1 and 4 of 9 participants (44.4%) in Arm 2 discontinued IMP dosing prematurely. The primary reasons for discontinuation included consent withdrawal for 1 participant in Arm 1 and 2 participants in Arm 2 and AE(s) for 2 participants in Arm 2. Of note, the 3 participants who withdrew consent also experienced TEAEs that were noted to have resulted in treatment withdrawal or that occurred at the same time as consent withdrawal. Mean baseline Phe levels were comparable in Arm 1 (1052±482 μM [range 507-1950 μM]) and Arm 2 (938±651 μM [range 367-2350 μM]), and 1 patient per arm was receiving concurrent sapropterin dihydrochloride at study entry (Table 11). Two patients participated in both arms of the study. Adherence to the study medication schedule was captured by counting unused doses and was 93.2% for SYNB1618 and 92.5% for SYNB1934.

TABLE 11
Patient Demographics

| | Arm 1, | Arm 2, |
|---|---|---|
| | SYNB1618 | SYNB1934v1 |
| Variable | (n = 11) | (n = 9) |

Demographics

| | | | |
|---|---|---|---|
| Age (years) | Mean ± SD | 29.4 ± 10.9 | 32.2 ± 8.9 |
| | Range | 18-50 | 23-47 |
| Sex (female, male) | | 6, 5 | 4, 5 |
| Baseline Phe level | Mean ± SD | 1052.4 ± 481.6 | 938.3 ± 651.4^{a} |
| (μM) | | | |
| | Range | 507.0-1950.0 | 367-2350^{a} |
| Baseline Phe intake | Mean ± SD | 1818.8 ± 1928.3 | 2209.2 ± 2585.5^{b} |
| (mg) | | | |
| | Range | 595-7155 | 550-8943^{b} |

| | | |
|---|---|---|
| Concomitant sapropterin use (n) | 1 | 1 |

SD = standard deviation.
^{a}Baseline fasting Phe available for 8/9 patients.
^{b}Baseline Phe intake available for 8/9 patients.

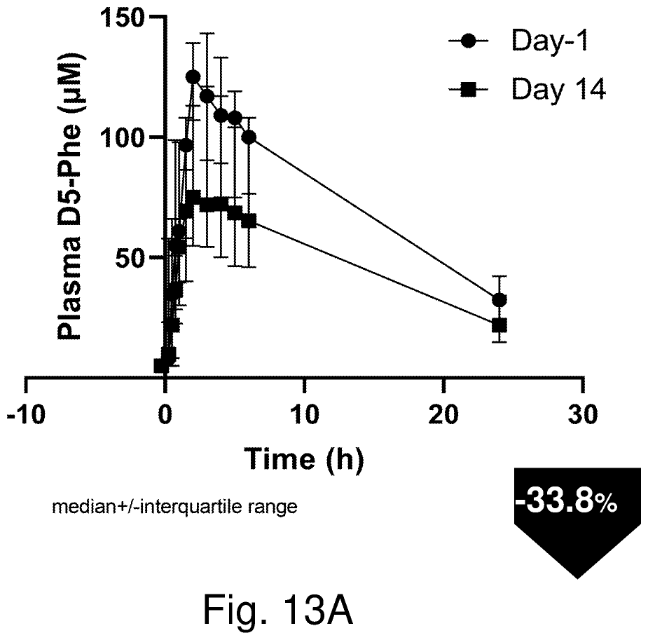

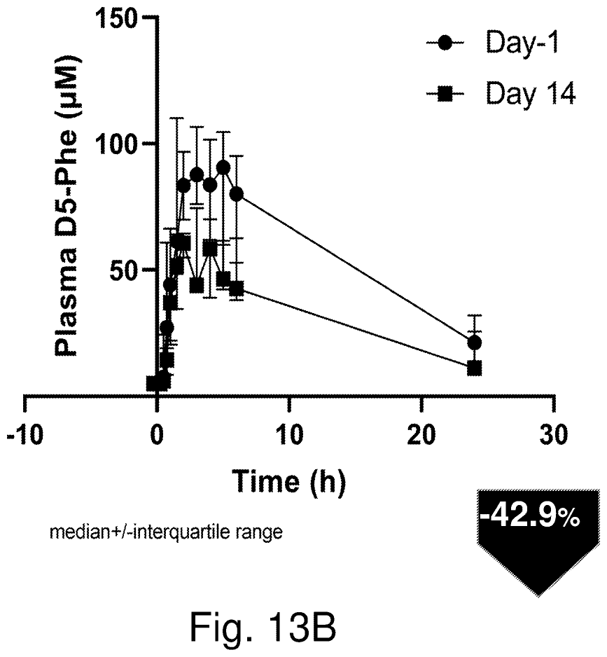

Results included achieving a reduction in area under the curve (AUC) of plasma levels of labeled D5-phenylalanine (D5-Phe), and in fasting plasma Phe levels from baseline for both strains. The D5-Phe tracer study showed significant decreases in plasma D5-Phe AUC₀₋₂₄, the primary efficacy endpoint, from baseline to Day 14. There was a least squares (LS) mean percent change from baseline of −34% (95% confidence interval [CI]: −54, −5) for SYNB1618 in Arm 1 and −43% (95% CI: −58, −22) for SYNB1934 in Arm 2 (FIGS. 13A and 13B).

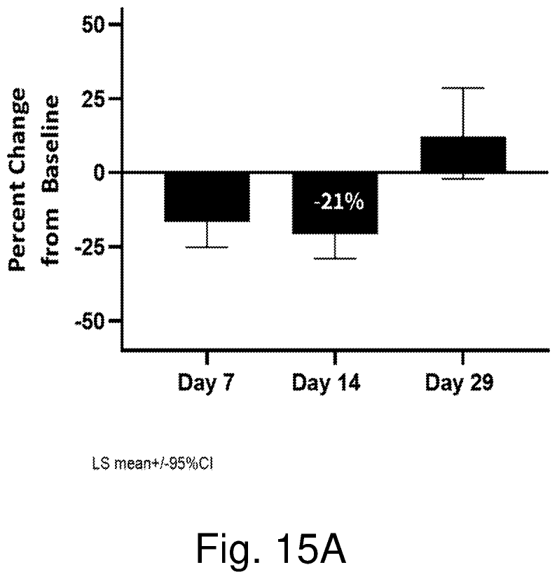

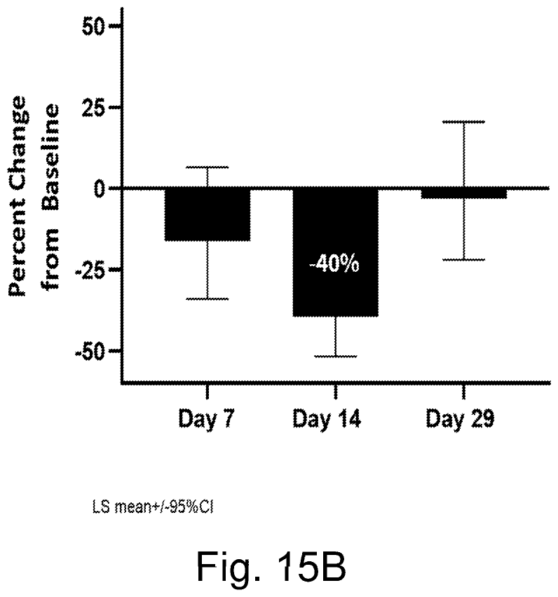

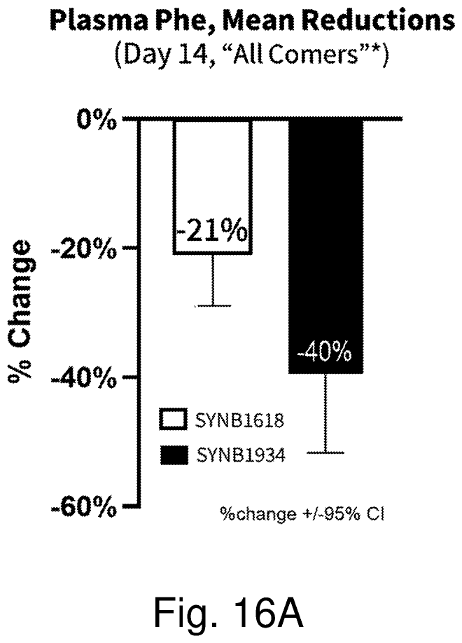

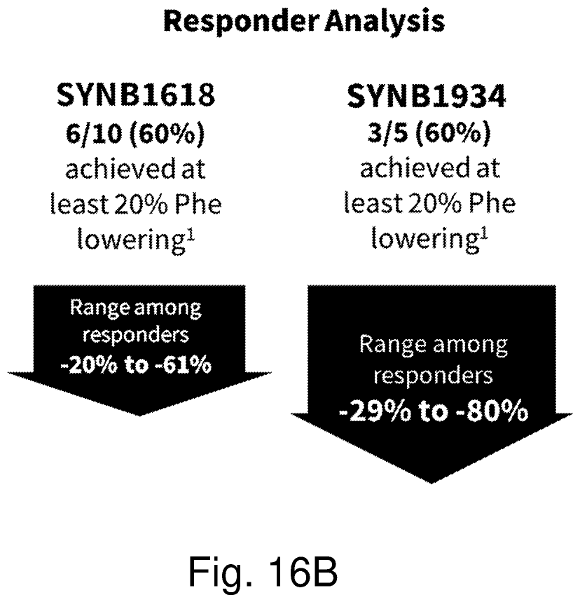

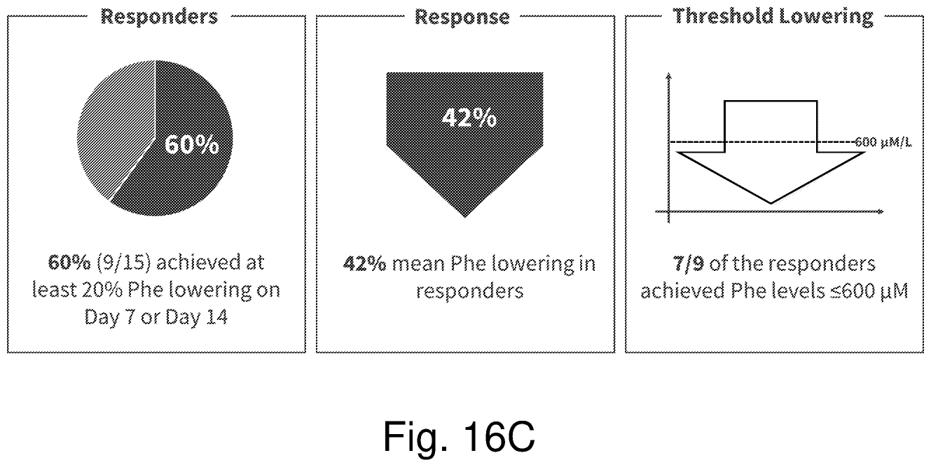

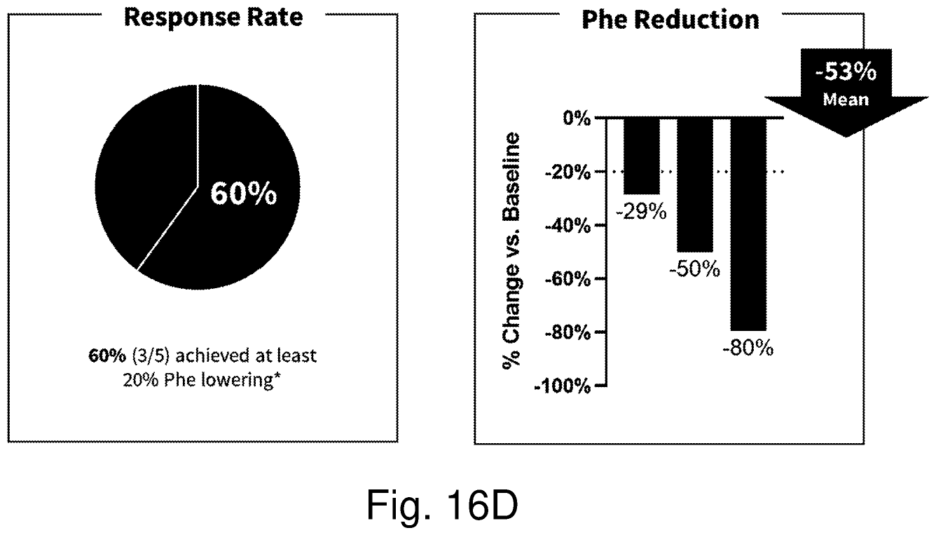

Both strains demonstrated a dose-dependent reduction in fasting plasma Phe (secondary endpoint). On an “all corners” basis, on Day 14, the estimated LS mean percent change from baseline was −21% (95% CI: −29, −11) in Arm 1 and −40% (95% CI: −52, −24) in Arm 2 (FIGS. 15A and 15B).

PKU patients with >20% reduction in the amount of Phe (e.g., μM plasma Phe) compared to baseline at Day 7 or Day 14 were considered responders. Six of the ten patients, i.e., 60% of patients, dosed with SYNB1618 and three of the five that have completed dosing with SYNB1934 met this criterion. In evaluating the response of all individuals in the trial, nine of 15 patients (60%) had a ≥20% reduction in fasting Phe, including 7 patients with fasting Phe below 600 μM. Among the responders, Phe was reduced by 20%-61% and 29%-80% for SYNB1618 and SYNB1934, respectively. Overall, the average reduction in the amount of Phe for responders was 42% (consolidating data for both strains). The average Phe reduction was 36% in SYNB1618 group and 53% in the SYNB1934 group.

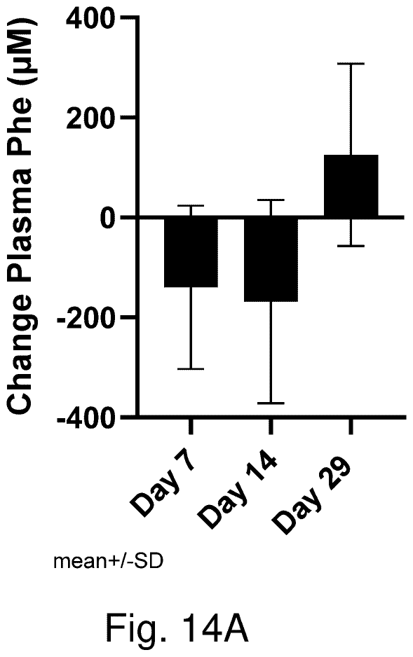

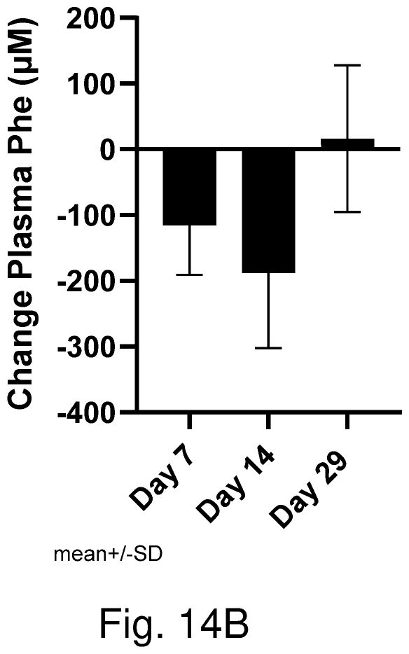

FIGS. 14A and 14B depicts the absolute reduction in the amount of Phe (e.g., μM plasma Phe) for patients on SYNB1618 compared to baseline at Day 7 or Day 14.

Among 20% responders (that occurred on either day 7 or day 14), 7 were for SYNB1618 and 3 for SYNB1934. For SYNB1618, the mean Phe lowering among responders was −330.143 uM. For SYNB1934 the mean Phe lowering among responders was −340.95 uM.

Additionally, further analysis revealed that 40% of patients had a 30% or greater reduction in amounts of plasma Phe compared to baseline at Day 7 or Day 14 (consolidating data from both strains). Patients with >30% reduction in the amount of Phe (e.g., μM plasma Phe) had an average Phe reduction of 50%.

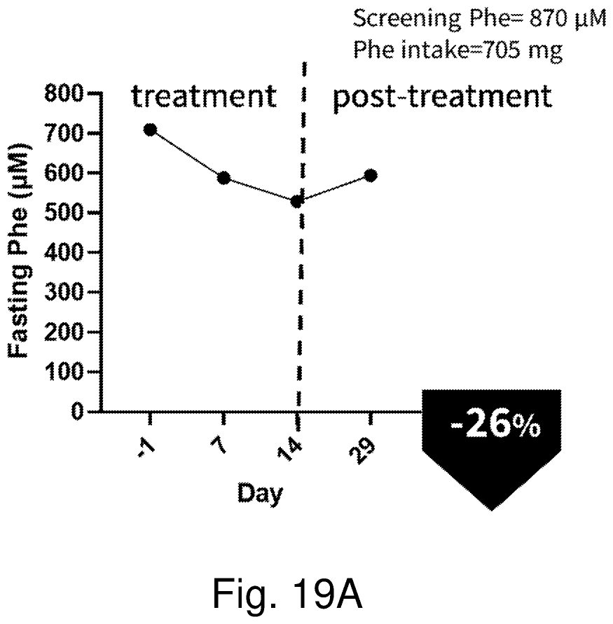

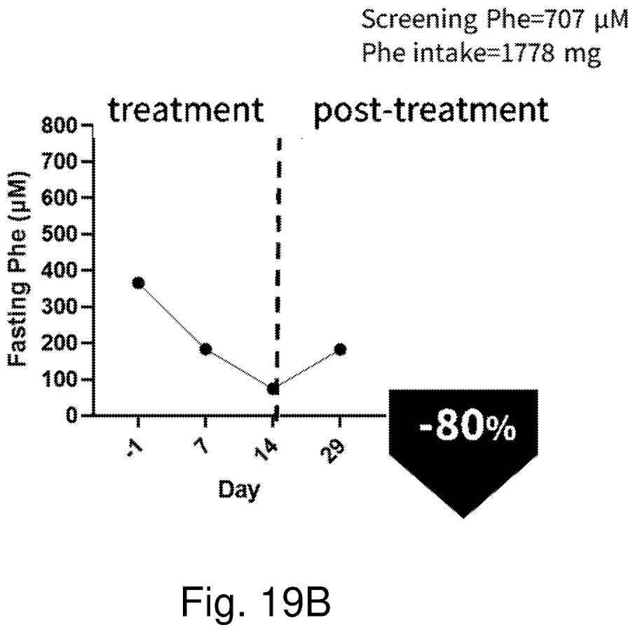

Results from patients who were already taking sapropterin (Kuvan®) at baseline, and then received SYNB1618 and SYNB1934, were consistent with the overall efficacy profile, demonstrating the potential for adjunctive use. Of note, the 2 patients, who were on concomitant sapropterin achieved notable Phe lowering during study treatment, with Day 14 reductions in plasma Phe of 26% following SYNB1618 dosing and 80% following SYNB1934 dosing; the latter patient achieved a Phe level on study that was within the normal physiological range (75 μmol/L) (FIGS. 19A-19D).

Fasting Phe values generally returned toward baseline at the Day 29 follow-up, with a mean change from baseline of 12% (95% CI: −2, 29) for Arm 1 and −3% (95% CI: −22, 21) for Arm 2.

The variation in response of fasting Phe to SYNB1618 and SYNB1934 administration could be due to PAH genotype, GI transit time or anatomy, Phe intake or other currently unrecognized factors.

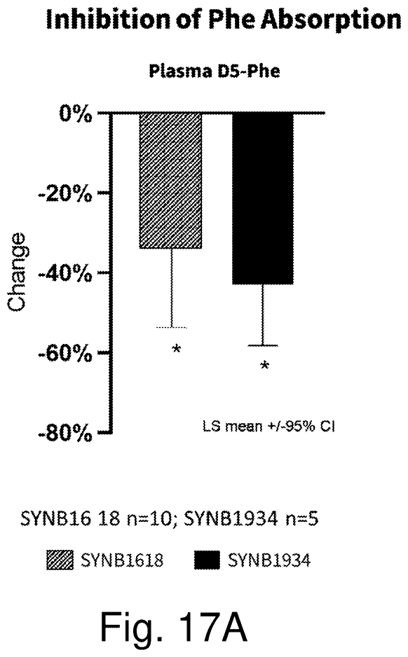

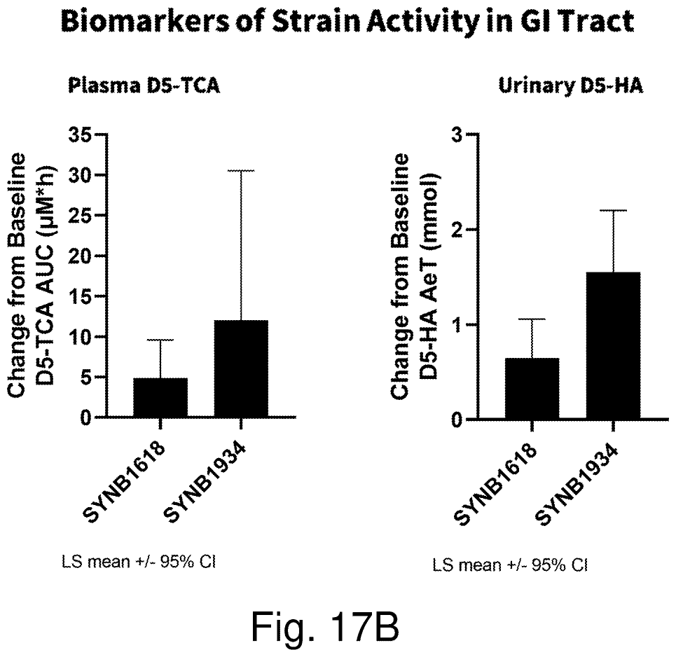

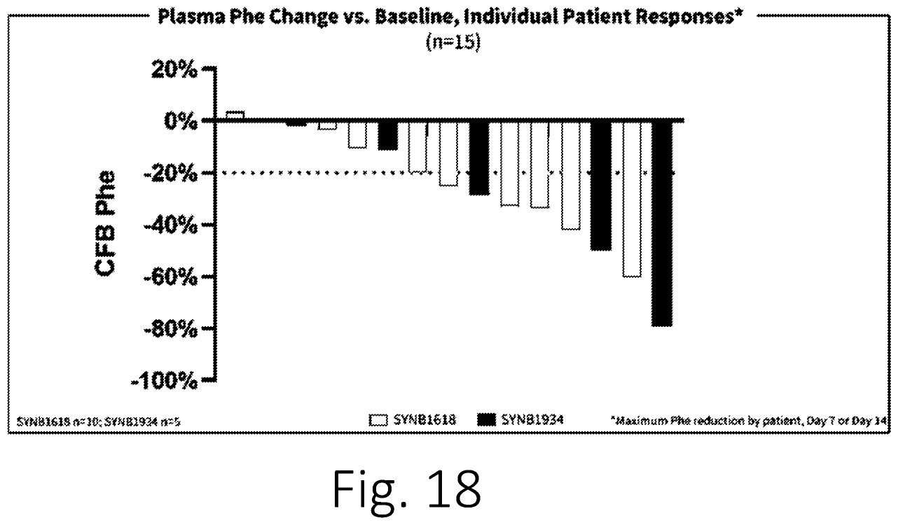

Dose-related increases in strain-specific biomarker production were also observed. At Day 14, the LS mean change from baseline in plasma D5-TCAAUC was 4.9 hr·μmol/L (95% CI: 2.5, 9.6) in Arm 1 and 12.1 hr·μmol/L (95% CI: 4.8, 30.5) in Arm 2, with a change from baseline in urine D5-HA Aet of 0.67 mmol (95% CI: 0.41, 1.10) in Arm 1 and 1.57 mmol (95% CI: 1.11, 2.22) in Arm 2 (FIG. 17B). The unlabeled biomarker study demonstrated production of Phe metabolites TCA in plasma and HA in urine on Days 7 and 15 following a low Phe breakfast, indicating strain activity on Phe resident in the gut lumen independent of dietary Phe intake. Table 12 summarizes the analysis of unlabeled Phe metabolites on Days 7 and 15 for Arm 1, SYNB1618 and Arm 2 SYNB1934 compared to baseline; (1) Change in plasma TCA AUC (uM*hr) for 0-4 h following overnight fast and low Phe breakfast on days 7 and 15; (2) Urinary total amount excreted for 0-4 h following overnight fast and low Phe breakfast on days 7 and 15.

TABLE 12
Change from Baseline in Plasma and Urine Phe metabolites from Biomarker study

| Parameter | Arm/strain | Day 7 | | 95% CI | Day 15 | | 95% CI |
|---|---|---|---|---|---|---|---|
| Plasma TCA | SYNB1618 | 1.56 | (n = 7)* | 1.30, 1.87 | 2.47 | (n = 8) | 1.40, 4.37 |
| | SYNB1934 | 0.42 | (n = 3) | 0.03, 5.63 | 2.13 | (n = 4) | 0.50. 9.15 |
| Urine HA | SYNB1618 | 0.31 | (n = 10)** | 0.07, 0.54 | 0.30 | (n = 9) | 0.20, 0.40 |
| | SYNB 1934 | 0.35 | (n = 4) | −0.4, 1.10 | 0.32 | (n = 5) | −0.12, 0.77 |

*data expressed as change from baseline to specified Day as LS mean in units of hr*μmol/L and noting number of patients evaluated. Patient number evaluated at each time point is shown.
**data expressed as change from baseline to specified Day as LS mean in mmol units and noting number of patients evaluated. Patient number evaluated at each time point is shown.
TCA = trans-cinnamic acid,
HA = hippuric acid,
LS = least squares,
AUC = area under the curve,
CI = confidence interval

All adverse events were mild or moderate in severity and were predominantly gastrointestinal (GI) in nature. Adverse events were similar across SYNB1618 and SYNB1934. There were no serious adverse events (SAEs). One patient receiving SYNB1618 discontinued due to disease-related anxiety and GI-related side effects. Of those receiving SYNB1934, two patients discontinued due to GI-related side effects; one withdrew consent during the baseline visit after experiencing adverse events, and another discontinued after experiencing facial flushing which was attributed to a possible allergic reaction.

Tyrosine levels, which can be deficient in PKU, remained in the safe range for all patients. All TEAEs were mild or moderate in severity and resolved after the last dose of IMP, with gastrointestinal events reported most frequently. A dose-related trend was observed in the incidence of TEAEs.

In conclusion, results were consistent and positive across all measured indicators of activity for both drug candidates including Area Under the Curve (AUC) D5-Phe, plasma D5-TCA and urinary D5-HA, with numerically greater changes observed for SYNB1934, consistent with previously shared results in Healthy Volunteers. Consistent with HV studies, SYNB1618 and SYNB1934 metabolize Phe in the GI tract in patients with PKU. There were no serious adverse events or deaths. Most commonly observed AEs were gastrointestinal. Consistent with preclinical data and head-to-head data in healthy volunteers, SYNB1934 has greater Phe metabolizing activity than SYNB1618. Phe lowering was seen with both SYNB1618 and SYNB1934 in a dose dependent manner. SYNB1934 may be effective in combination with sapropterin in patients who continue to have elevated Phe levels.

The synthetic biotics SYNB1934 and SYNB1618 lowered Phe levels in patients with PKU through the metabolism of Phe within the gut. The synthetic biotics demonstrated metabolism of dietary Phe in addition to metabolism of Phe in the context of a low Phe meal, suggesting access to a pool of Phe in the gut available through enterorecirculation.

The use of SYNB1934 in PKU provides a novel means of treatment with the potential to improve disease control and allow for effective Phe control with greater dietary Phe intake, although dietary management will remain foundational to therapy. SYNB1934 has advantages over existing oral pharmacotherapies for PKU that are dependent upon endogenous Phe metabolic systems and are ineffective for the most severe patients. In this study, there were two patients who used concomitant sapropterin and received an additive benefit from the use of synthetic biotics, suggesting that an additive benefit to co-administration of these medications. The use of PAL within the gut may provide benefits above systemic administration of the enzyme which leads to frequent immune responses.

PKU remains an incompletely treated disorder, despite a clear understanding of the risks of elevated Phe and the ability for dietary restriction to keep Phe values within a safe range. The challenges of dietary restriction in treating the PKU population lead to poor compliance and most adults are unable to reach target Phe levels. Elevated Phe in poorly controlled patients results in executive dysfunction, attention deficit, anxiety, depression, and other somatic symptoms, further exacerbating poor compliance. Therapies that could increase the intake of intact (Phe-containing) protein and reduce the reliance on supplemental Phe-free amino acids are of high importance to patients.

The microbiome contributes to health through metabolic pathways that are not encoded in the human genome, including the modification of vitamins and bile acids. To the inventors' knowledge, this is the first demonstration of efficacy for a synthetic biotic in the treatment of patients with a metabolic disorder. The EcN platform provides a protected envelope for introduction of enzymes or antigens into the gut where they can provide therapeutic benefit without digestion by intestinal proteases.

### Example 10. A Phase 3, Double-Blind, Placebo-Controlled, Randomized Withdrawal Study to Evaluate the Efficacy and Safety of SYNB1934v1 in Participants with PKU (SYNPHENY-3)

This is a 3-part, Phase 3 study consisting of a dose-escalating, open-label period (DEP; Part 1) of up to 15 weeks, followed by a 4-week, double-blind, placebo-controlled, randomized withdrawal period (RWP; Part 2), and an open-label extension (OLE; Part 3) of up to 36 months. The determination of the primary evidence of efficacy will be the comparison of fasting plasma level in participants using SYNB1934v1 versus placebo in the randomized withdrawal period (Part 2).

In the DEP, all participants will maintain a stable diet reflecting their baseline Phe intake and receive escalating doses of SYNB1934v1 from approximately 3 to 15 weeks to determine an individually titrated dose (iTD), which is defined as the highest dose the participants able to tolerate. A participant will be defined as having reached an iTD if they tolerate 3 weeks at a dose, regardless of whether other doses are tolerated.

Blood Phe level will be measured at each dose level at 3 weeks. A responder will be defined as a participant who achieves a ≥20% reduction in blood Phe level compared to DEP baseline. For inclusion in the primary efficacy population, response will be assessed at the third week of a patient's established iTD.

Participants who complete at least 3 weeks at their iTD during the DEP will enter a 4-week RWP. Participants will be randomized to receive SYNB1934v1 at their iTD determined in the DEP or placebo TID. Randomization will be stratified on baseline Phe level. Blood Phe level will be measured at Weeks 1, 3, and 4 of the RWP. Participants will remain on the same diet they consumed during the DEP.

Participants who complete the 4-week RWP may enter the OLE and receive SYNB1934v1 for up to 36 months at their iTD. The iTD in Parts 1 and 3 can differ. Participants will be allowed to modify their standard diet if their blood Phe level is <240 μmol/L, with guidance from the investigator. In an alternative protocol participants will be allowed to modify their standard diet if their Phe level is <360 μmol/L, with guidance from the investigator. 3-day dietary intake assessments will be conducted.

Once participants have established a stable dose in the OLE, a subgroup of participants optionally will complete an optional biomarker study to assess plasma levels of TCA. Blood will be drawn predose and 0.5, 1, 1.5, and 2 hours after administration of SYNB1934v1 to measure TCA in plasma.

The primary determination of efficacy will be based on the comparison of SYNB1934v1 versus placebo in the RWP. Safety will be evaluated by scheduled monitoring of adverse events, vital signs, and clinical laboratory measurements.

### Dosing and Dose Escalation

### Part 1: Dose-Escalating Open-Label (DEP):

In the DEP, all enrolled participants will maintain a stable diet reflecting their baseline Phe intake and receive escalating doses of SYNB1934v1 from approximately 3 to 15 weeks to determine an iTD, which is defined as the highest dose the participant is able to tolerate. A participant will be defined as having reached an iTD if they tolerate 3 weeks at a dose, regardless of whether other doses are tolerated.

SYNB1934v1 will be taken with a meal throughout the study. Participants will take a proton pump inhibitor (PPI) once per day (QD), recommended to occur before breakfast, during all parts of the study, starting at Day −7 of the DEP. In the DEP, participants will complete a dose ramp at the beginning of each dose level. The escalating dose levels are as follows: Dose 1: 3×10¹¹ live cells; Dose 2: 6×10¹¹ live cells; Dose 3: 1×10¹¹ live cells. During the dose ramp, SYNB1934v1 will be administered as shown in Table 13. Dosing will be with meals. On days with QD dosing, SYNB1934v1 should be taken with the mid-day meal. On days with twice per day (BID) dosing, SYNB1934v1 should be taken with the morning and evening meal.

TABLE 13
Dose Ramp Schedule
Dose Level 1

| Timing | D 1-9 | D 10-14 | D 15-21 |
|---|---|---|---|
| Dose (live cells) | partial | 3 × 10¹¹ | 3 × 10¹¹ |
| | 3 × 10¹¹* | | |
| Frequency | QD, D 1-3 | QD, D 10-11 | TID |
| | BID, D 4-6 | BID, D 12-14 | |
| | TID, D 7-9 | | |

Dose level 2

| Timing | Wk 4 | Wk 5 | Wk 6 |
|---|---|---|---|
| Dose (live cells) | 6 × 10¹¹ | 6 × 10¹¹ | 6 × 10¹¹ |
| Frequency | QD × 3 days | TID | TID |
| | BID × 4 days | | |

Dose Level 1
Dose Level 3

| Wk 7 | Wk 8 | Wk 9 | Wk 7 |
|---|---|---|---|
| Dose (live cells) | 1 × 10¹² | 1 × 10¹² | 1 × 10¹² |
| QD × 3 days | TID | TID | QD × 3 days |
| BID × 4 days | | | BID × 4 days |

BID = twice per day;
D = day;
QD = once per day;
TID = 3 times per day.
*e.g., 1 × 10¹¹ live cells

During Dose Level 1, participants will take a partial dose for the first 9 days. (approximately one-third of the suspension generated from a 3×10¹¹ live cell dose) before using the full dose of 3×10¹¹ cells. This dose will be prepared using a 3×10¹¹ sachet. This partial amount, only used during ramping, is prepared by making a 3×10¹¹ dose above and discarding approximately 70 mL (using a device with graduations in mL) and administering 30 mL of the dosing solution.

This is done to allow participants to gradually adjust to receiving a bolus of bacteria and minimize side effects. Participants will dose escalate to their iTD based on tolerability. If the participant tolerates the 3×10¹¹ dose after 3 weeks, and the investigator believes that further tolerance is likely, the investigator will escalate to the 6×10¹¹ dose. If the 6×10¹¹ is tolerated, the participant will continue with this dose for a total duration of 3 weeks. If the participant tolerates the 6×10¹¹ after 3 weeks, and the investigator believes that further tolerance is likely, the investigator will escalate to the 1×10¹² dose. The investigator can, with the medical monitor's assent, declare that the participant has reached iTD at any point during dose escalation, but the participant must stay on his or her iTD for 3 weeks prior to entering RWP.

A subset of the population will participate in a DEP weekly substudy, intended to enroll participants, to evaluate the range and variability of Phe and Tyr response at each dose level. Participants in this substudy will have weekly Phe and Tyr value assessments during the DEP.

If at any time during a 3-week dosing interval the participant or investigator considers a dose level to be intolerable, the dose may be de-escalated after discussion with the medical monitor. In this case, the participant will restart 3 weeks at the lower level iTD. After de-escalation, participants cannot escalate to a higher dose again during Part 1. In general, a dose is considered intolerable if the participant experiences Grade 3 or more severe adverse events (AEs) or Grade 2 gastrointestinal (GI) AEs that do not improve with continued dosing at the same dose level over time. If a dose is de-escalated due to a reason other than Grade 3 or greater, or Grade 2 GI AEs, or a second de-escalation is required, the medical monitor must be consulted.

Blood Phe level will be measured at each dose level after 3 weeks at that level. A responder will be defined as a participant who achieves a ≥20% reduction in blood Phe level compared to DEP baseline on their iTD. For inclusion in the RWP primary efficacy analysis population, response will be assessed at the third week of a participant's established iTD.

Participants who complete the DEP at their iTD for at least 3 weeks will enter a 4-week RWP.

### Part 2: Randomized Withdrawal:

In the RWP, participants will be randomized 1:1 to receive either SYNB1934v1 at their iTD established in the DEP or placebo three times daily (TID). Participants will remain on this dose of investigational medicinal product (IMP; i.e., SYNB1934v1 or placebo) for the duration of the RWP, unless they develop intolerance or meet other discontinuation criteria. Participants will remain on the same diet they consumed during the DEP. Blood Phe level will be measured at Week 1, 3 and 4 of the RWP. Participants who complete the 4-week RWP may enter the OLE and receive SYNB1934v1 for up to 36 months.

### Part 3: Open-Label Extension:

During the OLE, all participants will complete a dose ramp to their iTD over time guided by tolerability. The iTD in Part 3 may be different from the iTD in Part 1. The investigator may escalate the dose of SYNB1934v1 up to a maximum of 1×10¹² live cells based on tolerability; multiple attempts to escalate to a higher dose level are permitted per investigator discretion. Participants will be allowed to modify their standard diet if their blood Phe level is <240 μmol/L, with guidance from the investigator as outlined in the study-specific Diet Manual. The 3-day dietary intake assessments will continue per the Schedule of Events.

### Diet and Phenylalanine Monitoring:

During screening and for 3 consecutive days prior to all scheduled study visits and blood Phe draws, all participants will have a 3-day dietary intake assessment. A study dietitian will interview the participant and provide advice on maintaining a diet that is consistent with baseline diet throughout the study. Participants will follow their usual diet (including Phe, protein, and medical food intake) from baseline in the DEP to the end of the RWP.

During the DEP and RWP, the investigator will be notified if Phe is >1.5×the DEP baseline value, if Phe falls below 30 μmol/L or if Tyr falls below 20 μmol/L to allow for individual safety discontinuation.

During the OLE, participants will be allowed to modify their standard diet if their blood Phe level is <240 μmol/L, with guidance from the investigator. 3-day dietary intake assessments will continue.

### Study Duration:

The maximum time of study participation for a participant is planned to be approximately 6 months for screening through Part 2 and up to 36 months for Part 3, i.e., screening: up to 45 days; Part 1 (DEP): up to 15 weeks; Part 2 (RWP): 4 weeks; Part 3 (OLE): up to 36 months

### Inclusion Criteria:

1. Age≥18 years. Participants 12 to 17 years of age may be enrolled after the first 20 participants have completed the DEP, with approval of the data monitoring committee.
2. Able and willing to voluntarily complete the informed consent process (adults; parents/guardians) or informed assent process (minors).
3. Diagnosis of PKU and failure to maintain recommended blood Phe levels on existing management (sapropterin, sepiapterin and/or Phe-restricted diet), demonstrated by 2 blood Phe concentration measurements>360 mol/L, at least 7 days apart, during the screening period; or a single value during screening and history of blood Phe concentration>360 μmol/L on current treatment, as determined by central or local laboratory. Alternatively, either diagnosis of PKU and failure to maintain recommended blood Phe levels on existing management (sapropterin dihydrochloride, Sepiapterin, and/or Phe-restricted diet), demonstrated by 2 blood Phe concentration measurements>600 μmol/L, at least 7 days apart, during the screening period; or a single value during screening and history of blood Phe concentration consistently>600 μmol/L on current treatment, as determined by central or local laboratory. Alternatively, diagnosis of PKU and failure to maintain recommended blood Phe levels on existing management (sapropterin, sepiapterin and/or Phe-restricted diet), demonstrated by uncontrolled blood Phe level>360 μmol/L on current therapy any time during screening and uncontrolled blood Phe level>360 μmol/L on current therapy when taking the average of the 3 most recent Phe levels from the participant's medical history (inclusive of any screening values). All screening values must be obtained more than 7 days apart, as determined by central or local laboratory.
4. Females of childbearing potential must have a negative pregnancy test at screening and the end of DEP (in order to enter Part 2) and RWP (in order to enter Part 3) and be willing to have additional pregnancy tests during the study.
5. Sexually active female participants of childbearing potential must be willing to use an acceptable method of contraception while participating in the study and for 2 weeks after the last dose.
6. Stable diet including stable medical formula regimen (if used) for at least 1 month prior to screening.
7. If using sapropterin or sepiapterin, must be on a stable dose for at least 3 months.
8. Willing and able to continue current diet, sapropterin, sepiapterin, DEP, and RWP and to engage in all study activities.

### Exclusion Criteria:

1. Currently taking Palynziq® (pegvaliase-pqpz) (within 1 month of screening).
2. Acute or chronic medical, surgical, psychiatric, or social condition or laboratory abnormality that may increase participant risk associated with study participation, compromise adherence to study procedures and requirements, and, in the judgment of the investigator, would make the participant inappropriate for enrollment. A known or suspected diagnosis of DNAJC12 deficiency, biopterin synthesis deficiency, diagnosis of irritable bowel syndrome.
3. Intolerance of or allergic reaction to E. coli Nissle or any of the ingredients in SYNB1934v1 or placebo formulations, or an allergy to cinnamon. Known intolerance to proton pump inhibitors and H2 blockers.
4. Currently taking or plans to take any type of systemic (e.g., oral or intravenous) antibiotic within 28 days prior to the first dose of IMP through final safety assessment in RWP, including planned surgery, hospitalizations, dental procedures, or interventional studies that are expected to require antibiotics. Exception: topical antibiotics are allowed.
5. Pregnant, planning to become pregnant, or breastfeeding.
6. Current participation in any other investigational drug study or use of any investigational agent within 30 days or 5 half-lives (whichever is longer) prior to screening.
7. Ever received gene therapy for treatment of PKU.

### Treatment

Within this protocol IMP refers to SYNB1934v1 or placebo. SYNB1934v1 IMP consists of powder for oral suspension packaged in sachets, to be resuspended in water or apple juice. During dose preparation, the powder will be resuspended in 100 mL of water or apple juice. The prepared dose is to be administered within 15 minutes of resuspension.

Placebo will be manufactured using an inactive powder that is color matched to the SYNB1934v1 drug product. In order to maintain study blinding during the RWP, placebo will be packaged, labeled, stored, and administered in an identical manner to SYNB1934v1.

Study participants will prepare the individual doses at home. They will be instructed on the required procedures by trained study personnel and will receive written instructions on IMP preparation and storage.

Concomitant medications can be administered at the investigator's discretion to conform to standard practice. Given the gut-restricted mechanism of action, SYNB1934v1 has a low likelihood for eliciting clinically relevant drug-drug interactions; however, no traditional drug-drug interaction studies have been conducted. All concomitant medications and dietary supplements will be recorded.
a. During participation in the study, participants will take a proton pump inhibitor (PPI, esomeprazole 20 mg or equivalent) QD (recommended to occur before breakfast) starting 7 days before first dose of IMP in Part 1. The PPI should be taken at the same time each day, even if no meal is consumed. If participants are already on a PPI regimen consistent with instructions in the package insert, they may continue on that and not switch to esomeprazole. If participants cannot tolerate a PPI, they can use an H2 blocker per investigator discretion. The dose of PPI or H2 blocker should remain stable for Parts 1 and 2 of the study; if a dose adjustment to the PPI or H2 blocker is required in Part 3, the investigator must discuss with the medical monitor (unless the adjustment is required due to an adverse event). If a PPI or H2 dose is missed, it should be taken as soon as realized and then resume the normal before-breakfast QD or BID schedule, according to the locally approved PPI or H2 label.

During participation in the study, participants will be provided with ondansetron, an approved medication for chemotherapy-associated, radiotherapy-associated, and post-operative nausea and vomiting. The use of this medication in the study is optional and at the discretion of the investigator.

### Pharmacodynamic Assessments

### Blood Phenylalanine and Tyrosine:

Blood Phe and Tyr samples will be drawn 2.5 to 5 hours after the last meal. Phe samples will be drawn in duplicate, using separate tubes from the same venipuncture, and analyzed separately. The time of the last meal will be recorded when taking Phe samples. During the DEP, blood Phe and Tyr will be measured at Week 3 of each dose level. Blood Phe and Tyr will be measured at Weeks 1, 3, and 4 during the RWP. During the OLE, blood Phe and Tyr will be measured quarterly. Blood Phe and Tyr will also be measured at early termination.

### TCA Sub-Study

In an alternative study, an optional TCA Sub-study is added; Prior to the first dose in the DEP, a single exploratory plasma sample will be drawn from all participants. This sample serves as a TCA baseline. The TCA baseline sample may only be analyzed in participants of the TCA sub-study. During the OLE, a subgroup of patients will complete an optional biomarker study to assess plasma levels of TCA once they have reached a stable dose of SYNB1934v1. After an overnight fast, participants will consume a protein shake. Blood will be drawn at baseline and 0.5, 1, 1.5, and 2 hours after subsequent administration of SYNB1934v1 to measure TCA in plasma.

### Phenylalanine and Tyrosine Substudy

Participants will have the option to enroll in a substudy to evaluate the range and variability of Phe and Tyr response at each dose level. Participants in this substudy will have weekly Phe and Tyr value assessments during the DEP. These weekly values may be performed during a 3-day window around the 8th and 15th day of each dose. These samples will be drawn 2.5 to 5 hours after the last meal. Phe samples will be drawn in duplicate, using separate tubes from the same venipuncture, and analyzed separately. The time of the last meal will be recorded when taking Phe samples.

### Cognitive Function and Attention Assessment:

In an alternative study, a Cognitive Function and Attention Assessment is added. Cognitive function will be assessed using the CANTAB. The CANTAB cognitive assessment is a battery of computerized tests designed to detect both cognitive impairment and enhancement. The selected battery of 3 tests will measure cognitive function. A spatial working memory (SWM) task will be used to assess the ability of the participants to retain spatial information and manipulate it in working memory. A rapid visual information processing (RVP) task will be used to assess sustained attention, outputting measures of response accuracy, target sensitivity and reaction times. Stop signal task (SST) will be used to assess response inhibition. All tests will be completed electronically. A practice session will be completed during screening.

Attention will be assessed in adults using the ADHD-RS-IV IA. This instrument is a self-administered symptom checklist consisting of the 18 questions aimed at evaluating ADHD-related symptoms. The youth version of the ADHD RS-IV, which is observer rated, will be used for participants<18 years of age.

### Safety Assessments

The safety and tolerability of SYNB1934v1: incidence and severity of adverse events will be assessed through changes from baseline in clinical laboratory parameters and changes from baseline in vital signs measurements.

### Vital Signs, Weight, Height, and Electrocardiograms:

Semi-supine vital signs (systolic blood pressure, diastolic blood pressure, pulse, and body temperature), weight, and height will be collected. Participants are required to remain in the semi-supine position for at least 5 minutes prior to obtaining vital signs. Body mass index will be calculated based on weight and height. Supine single 12-lead electrocardiograms (ECGs) will be performed at baseline.

### Adverse Events:

Adverse events will be assessed continuously by direct observation and participant interviews. An AE is any untoward medical occurrence, including the exacerbation of a preexisting condition, in a participant administered a pharmaceutical product, regardless of causality The severity of AEs will be evaluated using the National Cancer Institute Common Terminology Criteria for Adverse Events (NCI CTCAE). All AEs occurring from the time a participant signs the ICF through the follow-up period will be recorded, regardless of causal assessment to IMP.

The severity rating of an AE refers to its intensity. The severity of each AE will be categorized using the NCI CTCAE. For any term that is not specifically listed in the CTCAE scale, intensity should be assigned a grade of 1 through 5 using the following CTCAE guidelines: Grade 1: Mild; asymptomatic or mild symptoms; clinical or diagnostic observations only; intervention not indicated; Grade 2: Moderate; minimal, local or noninvasive intervention indicated; limiting age-appropriate instrumental activities of daily living; Grade 3: Severe or medically significant but not immediately life-threatening; hospitalization or prolongation of hospitalization indicated; disabling; limiting self-care activities of daily living. Grade 4: Life-threatening consequences; urgent intervention indicated. Grade 5: Death related to AE.

Medical judgment should be used to determine the cause of the AE, considering all relevant factors such as (but not limited to) the underlying study indication, coexisting disease, concomitant medication, relevant history, pattern of the AE, temporal relationship to IMP, and dechallenge or rechallenge.

Exceptions to CTCAE criteria will be implemented for grading certain GI-related TEAEs, as detailed in Table 14.

TABLE 14
Severity Assessment of Diarrhea and Nausea

| Grade | Diarrhea^{a} | Nausea |
|---|---|---|
| 1 | Increase of diarrhea, <3 liquid | Loss of appetite without |
| | stools a day over baseline; mild | alteration in eating habits |
| | increase in ostomy output | and/or with use of planned |
| | compared to baseline | anti-nausea medication |
| 2 | Diarrhea ≥ 3 liquid stools | Use of unplanned concomitant |
| | daily and limiting instrumental | medications to increase oral |
| | ADLs | intake |
| 3 | Diarrhea requiring hospital- | Inadequate oral caloric or |
| | ization or limiting self-care | fluid intake; tube feeding, |
| | ADLs | TPN or hospitalization |
| | | indicated (unchanged from |
| | | CTCAE) |
| 4 | Life-threatening consequences; | Not applicable |
| | urgent intervention indicated | |
| | (unchanged from CTCAE) | |
| 5 | Death (unchanged from | Not applicable |
| | CTCAE) | |

ADLs = activities of daily living; CTCAE = Common Terminology Criteria for Adverse Events.
^{a}Diarrhea is defined as liquid stools without consistency in shape.

### Endpoint Analyses:

The primary determination of efficacy will be based on the comparison of SYNB1934v1 versus placebo in the RWP. Safety will be evaluated by scheduled monitoring of adverse events, vital signs, and clinical laboratory measurements.

A responder is defined as a participant who achieves a ≥20% reduction from baseline in blood Phe level at the third week of a participant's iTD of SYNB1934v1 in Part 1.

To evaluate efficacy of SYNB1934v1 versus placebo in the responder population, the baseline for blood Phe level in each of the 3 parts of the study will be defined as the mean of the duplicate blood Phe level measurements at obtained immediately prior to administration of the first dose of each part. If only one blood Phe level measurement is available, then that measure will be used as baseline. Similarly, for the determination of responders and participants who achieve a specific reduction in Phe (e.g., Phe level≤360 μmol/L at any time in the DEP, or a ≥20% reduction from baseline) the mean of the duplicate blood Phe level measurements at the applicable post-baseline timepoint (e.g., Week 3) will be used. If only one of the duplicate blood Phe levels is available at a particular post-baseline timepoint, then that measurement will be used.

### Primary Outcome Measure:

### DEP (Part 1)

1. To assess the percentage change in blood phenylalanine (Phe) level. Time Frame: up to 15 weeks. Endpoint is Percent change from baseline in blood Phe level at last measurement of the iTD of SYNB1934v1

### RWP (Part 2)

2. To evaluate efficacy of SYNB1934v1 versus placebo in the responder population through the change from baseline to Week 4 in blood Phe level. Time Frame: 4 weeks. Endpoint is Change from baseline to Week 4 in blood Phe level.

### Secondary Outcome Measure:

### DEP (Part 1)

3. To assess the absolute change in Phe level. Time Frame: up to 15 weeks. Endpoint is: Change from baseline in blood Phe level at last measurement of the iTD of SYNB1934v1.
4. To assess proportion of participants with a ≥20% reduction in blood Phe level. Time Frame: up to 15 weeks. End point is A ≥20% reduction from baseline in blood Phe level at any time of the iTD of SYNB1934v1; and/or A ≥20% reduction from baseline in blood Phe level at any time in the DEP

### RWP (Part 2)

5. To evaluate the efficacy of SYNB1934v1 versus placebo in the responder population with regard to the proportion of participants with a blood Phe level≤360 μmol/L. Time Frame: 4 weeks. Endpoint is Blood Phe level≤360 μmol/L at Week 4.
6. To evaluate the efficacy of SYNB1934v1 versus placebo in the responder population with regard to change from DEP baseline in blood Phe level, Time Frame: 4 weeks. Endpoint is Change from DEP baseline to Week 4 in blood Phe level.
7. To evaluate efficacy of SYNB1934v1 versus placebo in the responder population with regard to percent change from DEP baseline in blood Phe level at Week 4. Time Frame: 4 weeks. Endpoint is Percent change from DEP baseline in blood Phe level at Week 4.

DEP objectives and endpoints are listed in Table 15.

TABLE 15
Dose Escalating, Open-Label Period (DEP)

| Objective | Endpoint |
|---|---|
| Primary: To assess the percent | Percent change from baseline in |
| change in blood Phe level | blood Phe level at last |
| | measurement of the iTD of |
| | SYNB1934v1 |
| Key Secondary: | |
| To assess the absolute change in | Change from baseline in blood Phe |
| Phe level | level at last measurement of the |
| | iTD of SYNB1934v1 |
| To assess proportion of participants | A ≥ 20% reduction from baseline |
| with a ≥20% reduction in blood | in blood Phe level at any time of |
| Phe level | the iTD of SYNB1934v1 |
| | A ≥ 20% reduction from baseline |
| | in blood Phe level at any time in |
| | the DEP |
| Other Secondary: | |
| To assess proportion of participants | Blood Phe level ≤ 360 μmol/L at |
| achieving blood Phe | any time of the iTD of |
| level ≤ 360 μmol/L | SYNB1934v1 |
| | Blood Phe level ≤ 360 μmol/L at |
| | any time in the DEP |
| To assess the safety and tolerability | Incidence and severity of adverse |
| of SYNB1934v1 | events |
| | Changes from baseline in clinical |
| | laboratory parameters |
| | Changes from baseline in |
| | vital signs measurements |
| Exploratory: | |
| To determine the responder | A ≥ 20% reduction from baseline |
| population for Part 2 | in blood Phe level at the third week |
| | of a participant's last 3 weeks |
| | at the iTD of SYNB1934v1 |
| To assess the absolute and relative | Dose level at first blood Phe |
| change in blood Phe level | level ≤ 360 μmol/L |
| | Dose level at first ≥ 20% reduction |
| | in blood Phe level |
| | Blood Phe level ≤ 360 μmol/L at |
| | any time of the iTD of |
| | SYNB1934v1 |
| To assess proportion of participants | Blood Phe level ≤ 600 μmol/L at |
| achieving blood Phe | any time of the iTD of |
| level ≤ 600 μmol/L with a | SYNB1934v1 |
| baseline value > 600 μmol/L | Blood Phe level ≤ 600 μmol/L at |
| | any time in the DEP |
| To assess the range and variability | Change in weekly blood Phe |
| of Phe response at each dose level | levels during each step of the |
| | dose ramp |

Note:
baseline and week numbering are specific to study Part 1, unless explicitly noted otherwise in the endpoint.

At each of the scheduled assessments, blood Phe will be drawn in duplicate.

### Example 11: Exemplary Sequences

TABLE 16

| Description | Sequence |
|---|---|
| PheP | MKNASTVSEDTASNQEPTLHRGLHNRHIQLIALGGAIGTGLFLGIGPAI |
| E. coli | QMAGPAVLLGYGVAGIIAFLIMRQLGEMVVEEPVSGSFAHFAYKYW |
| SEQ ID | GPFAGFLSGWNYWVMFVLVGMAELTAAGIYMQYWFPDVPTWIWAA |
| NO: 509 | AFFIIINAVNLVNVRLYGETEFWFALIKVLAIIGMIGFGLWLLFSGHGG |
| | EKASIDNLWRYGGFFATGWNGLILSLAVIMFSFGGLELIGITAAEARD |
| | PEKSIPKAVNQVVYRILLFYIGSLVVLLALYPWVEVKSNSSPFVMIFHN |
| | LDSNVVASALNFVILVASLSVYNSGVYSNSRMLFGLSVQGNAPKFLT |
| | RVSRRGVPINSLMLSGAITSLVVLINYLLPQKAFGLLMALVVATLLLN |
| | WIMICLAHLRFRAAMRRQGRETQFKALLYPFGNYLCIAFLGMILLLM |
| | CTMDDMRLSAILLPVWIVFLFMAFKTLRRK |
| LAAD | MNISRRKLLLGVGAAGVLAGGAALVPMVRRDGKFVEAKSRASFVEG |
| Proteus | TQGALPKEADVVIIGAGIQGIMTAINLAERGMSVTILEKGQIAGEQSGR |
| mirabilis | AYSQIISYQTSPEIFPLHHYGKILWRGMNEKIGADTSYRTQGRVEALA |
| SEQ ID | DEKALDKAQAWIKTAKEAAGFDTPLNTRIIKGEELSNRLVGAQTPWT |
| NO: 510 | VAAFEEDSGSVDPETGTPALARYAKQIGVKIYTNCAVRGIETAGGKIS |
| | DVVSEKGAIKTSQVVLAGGIWSRLFMGNMGIDIPTLNVYLSQQRVSG |
| | VPGAPRGNVHLPNGIHFREQADGTYAVAPRIFTSSIVKDSFLLGPKFM |
| | HLLGGGELPLEFSIGEDLFNSFKMPTSWNLDEKTPFEQFRVATATQNT |
| | QHLDAVFQRMKTEFPVFEKSEVVERWGAVVSPTFDELPIISEVKEYPG |
| | LVINTATVWGMTEGPAAGEVTADIVMGKKPVIDPTPFSLDRFKK* |

What is claimed is:

1. A method of reducing phenylalanine in a subject, comprising administering to the subject a genetically engineered bacterium comprising:
a. one or more heterologous gene(s) encoding a phenylalanine ammonia lyase (PAL),
b. one or more heterologous gene(s) encoding a phenylalanine transporter, and
c. one or more heterologous gene(s) encoding a L-amino acid deaminase (LAAD),
wherein the subject achieves a reduction in a phenylalanine level after administration as compared to a baseline level of phenylalanine in the subject before administration.

2. A method of reducing hyperphenylalaninemia in a subject, comprising administering to the subject a genetically engineered bacterium comprising:
a. one or more heterologous gene(s) encoding a phenylalanine ammonia lyase (PAL),
b. one or more heterologous gene(s) encoding a phenylalanine transporter, and
c. one or more heterologous gene(s) encoding a L-amino acid deaminase (LAAD),
wherein the subject achieves a reduction in a phenylalanine level after administration as compared to a baseline level of phenylalanine in the subject before administration.

3. A method of treating phenylketonuria in a subject, comprising administering to the subject a genetically engineered bacterium comprising:
a. one or more heterologous gene(s) encoding a phenylalanine ammonia lyase (PAL),
b. one or more heterologous gene(s) encoding a phenylalanine transporter, and
c. one or more heterologous gene(s) encoding a L-amino acid deaminase (LAAD),
wherein the subject achieves a reduction in a phenylalanine level after administration as compared to a baseline level of phenylalanine in the subject before administration.

4. The method of any one of claims 1-3, wherein the phenylalanine level is a blood phenylalanine level.

5. The method of any one of claims 1-3, wherein the phenylalanine level is a plasma phenylalanine level.

6. The method of any one of claims 1-5, wherein the subject achieves an improvement in at least one symptom after the administering, wherein the symptom is irritability, fatigue, convulsions, skin rashes, attention deficit, executive dysfunction, mood disorder and/or musty body odor.

7. The method of any one of claims 1-6, wherein the subject achieves at least a 5%, at least a 10%, at least a 15%, at least a 20%, at least a 25%, at least a 30%, at least a 35%, at least a 40%, at least a 45%, at least a 50%, at least a 55%, at least a 60% or at least an 80% reduction in the phenylalanine level after administration as compared to the baseline level in the subject before administration.

8. The method of any one of claims 1-7, wherein the subject achieves at least a 20% reduction in phenylalanine level after administration as compared to the baseline level in the subject before administration.

9. The method of any one of claims 1-8, wherein the subject achieves an increase in a level of trans-cinnamic acid (TCA) level after administration as compared to a baseline level of TCA in the subject before administration.

10. The method of any one of claims 1-9, wherein the subject is capable of consuming at least about 10%, at least about 20%, at least about 25%, at least about 30%, at least about 40%, at least about 50%, at least about 60%, at least about 70%, at least about 75%, at least about 80%, at least about 85%, at least about 90%, at least about 95%, or more protein while maintaining or lowering blood or plasma phenylalanine as compared to before administration of the genetically engineered bacterium.

11. The method of any one of claims 1-10, wherein the subject is capable of consuming at least about 2-fold, at least about 3-fold, at least about 4-fold or more protein while maintaining or lowering blood or plasma phenylalanine as compared to before administration of the genetically engineered bacterium.

12. The method of any one of claims 1-11, wherein the subject is capable of consuming at least 1 g, at least 2 g, at least 3 g, at least 4 g, at least 5 g, at least 6 g, at least 7 g, at least 8 g, at least 9 g, or at least 10 g more protein while maintaining or lowering blood or plasma phenylalanine as compared to before administration of the genetically engineered bacterium.

13. The method of any one of claims 1-12, wherein the subject is capable of consuming at least 10 g, at least 11 g, at least 12 g, at least 13 g, at least 14 g, at least 15 g, at least 16 g, at least 17 g, at least 18 g, at least 19 g, or at least 20 g more protein while maintaining or lowering blood or plasma phenylalanine as compared to before administration of the genetically engineered bacterium.

14. The method of any one of claims 1-13, wherein the genetically engineered bacterium comprises:
a. one or more heterologous gene(s) encoding a phenylalanine ammonia lyase (PAL), operably linked to a promoter that is induced under low-oxygen or anaerobic conditions,
b. one or more heterologous gene(s) encoding a phenylalanine transporter, operably linked to a promoter that is induced under low-oxygen or anaerobic conditions, and
c. one or more heterologous gene(s) encoding a L-amino acid deaminase (LAAD), operably linked to an arabinose-inducible promoter.

15. The method of any one of claims 1-13, wherein the genetically engineered bacterium comprises:
a. one or more heterologous gene(s) encoding a phenylalanine ammonia lyase (PAL), operably linked to an IPTG inducible promoter,
b. one or more heterologous gene(s) encoding a phenylalanine transporter, operably linked to an IPTG inducible promoter, and
c. one or more heterologous gene(s) encoding a L-amino acid deaminase (LAAD), operably linked to an arabinose-inducible promoter.

16. The method of any one of claims 1-15, comprising administering to the subject the genetically engineered bacterium at a dose of about 1×10¹¹, about 2×10¹¹, about 3×10¹¹, about 4×10¹¹, about 5×10¹¹, about 6×10¹¹, about 7×10¹¹, about 8×10¹¹, or about 9×10¹¹ cells, as determined by live cell counting.

17. The method of any one of claims 1-16, comprising administering to the subject the genetically engineered bacterium at a dose of about 1×10¹², about 2×10¹², about 3×10¹², about 4×10¹², about 5×10¹², about 6×10¹², about 7×10¹², about 8×10¹², or about 9×10¹² cells, as determined by live cell counting.

18. The method of any one of claims 1-17, comprising administering to the subject the genetically engineered bacterium for a first dosing interval, a second dosing interval, and/or a third dosing interval.

19. The method of claim 18, wherein the first dosing interval, the second dosing interval, and/or the third dosing interval comprise(s) administering to the subject a dose of genetically engineered bacterium once per day (QD) for a set amount of days, then said dose twice per day (BID) for a set amount of days, and then said dose three times per day (TID) for a set amount of days.

20. The method of claim 18, wherein the first dosing interval comprises administering to the subject a one-third dose of genetically engineered bacterium once per day (QD) for a set amount of days, then a one-third dose twice per day (BID) for a set amount of days, and then a one-third dose three times per day (TID) for a set amount of days.

21. The method of claim 18 or 20, wherein the second dosing interval comprises administering to the subject a full dose of genetically engineered bacterium once per day (QD) for a set amount of days, then the full dose twice per day (BID) for a set amount of days, and then the full dose three times per day (TID) for a set amount of days.

22. The method of any one of claims 18, 20, or 21, wherein the third dosing interval comprises administering to the subject a full dose of genetically engineered bacterium once per day (QD) for a set amount of days, then the full dose twice per day (BID) for a set amount of days, and then the full dose three times per day (TID) for a set amount of days.

23. The method of claim 22, wherein the full dose of the second dosing interval and the full dose of the third dosing interval are different doses.

24. The method of any one of claims 18-23, wherein a dose of genetically engineered bacterium is administered once per day (QD) for at least 1 day, at least 2 days, at least 3 days, at least 4 days, at least 5 days, at least 6 days, or at least 7 days in the first dosing interval, the second dosing interval, and/or the third dosing interval.

25. The method of any one of claims 18-24, wherein a dose of the genetically engineered bacterium is administered twice per day (BID) for at least 1 day, at least 2 days, at least 3 days, at least 4 days, at least 5 days, at least 6 days, or at least 7 days in the first dosing interval, the second dosing interval, and/or the third dosing interval.

26. The method of any one of claims 18-25, wherein a dose of the genetically engineered bacterium is administered three times per day (TID) for at least 1 day, at least 2 days, at least 3 days, at least 4 days, at least 5 days, at least 6 days, at least 7 days, or the remainder of days in the first dosing interval, the second dosing interval, and/or the third dosing interval.

27. The method of any one of claims 18-26, wherein the first dosing interval, the second dosing interval, and/or the third dosing interval are at least 1 week, at least 2 weeks, at least 3 weeks, at least 4 weeks, at least 5 weeks, at least 6 weeks, at least 7 weeks, at least 8 weeks, at least 9 weeks, at least 10 weeks, or at least 11 weeks.

28. The method of any one of claims 18-27, wherein the first dosing interval, the second dosing interval, and/or the third dosing interval combined are at least 6 weeks, at least 7 weeks, at least 8 weeks, at least 9 weeks, at least 10 weeks, at least 11 weeks, at least 12 weeks, at least 13 weeks, at least 14 weeks, or at least 15 weeks.

29. The method of any one of claims 18-28, wherein the dose of genetically engineered bacterium administered to the subject is:
a. about 1×10¹¹ and/or 3×10¹¹ cells in the first dosing interval;
b. about 6×10¹¹ cells in the second dosing interval; and/or
c. about 1×10¹² cells in the third dosing interval.

30. The method of claim 29, wherein the genetically engineered bacterium administered to the subject in the first dosing interval is a one-third dose of about 1×10¹¹ cells and/or a full dose of about 3×10¹¹ cells.

31. The method of any one of claims 18-30, wherein the first dosing interval is 21 days in length, and the genetically engineered bacterium is administered to the subject:
a. at a dose of about 1×10¹¹ cells once per day for days 1-3;
b. at a dose of about 1×10¹¹ cells twice per day for days 4-6;
c. at a dose of about 1×10¹¹ cells three times per day for days 7-9;
d. at a dose of about 3×10¹¹ cells once per day for days 10-11;
e. at a dose of about 3×10¹¹ cells twice per day for days 12-14; and
f. at a dose of about 3×10¹¹ cells three times per day for days 15-21,
as determined by live cell counting.

32. The method of any one of claims 18-31, wherein the second dosing interval is 21 days in length, and the genetically engineered bacterium is administered to the subject at a dose of about 6×10¹¹ cells, as determined by live cell counting:
a. once per day for days 1-3;
b. twice per day for days 4-7; and
c. three times per day for days 8-21.

33. The method of any one of claims 18-32, wherein the second dosing interval is at least 21 days in length, and the genetically engineered bacterium is administered to the subject at a dose of about 1×10¹² cells, as determined by live cell counting:
a. once per day for days 1-3;
b. twice per day for days 4-7; and
c. three times per day for at least days 8-21.

34. The method of any one of claims 18-33, comprising administering to the subject genetically engineered bacterium:
a. for the first dosing interval only,
b. for the first dosing interval and the second dosing interval, only, or
c. for the first dosing interval, the second dosing interval, and the third dosing interval.

35. The method of any one of claims 1-34, comprising administering to the subject a formulation of genetically engineered bacterium comprising the genetically engineered bacterium, sucralose, sodium bicarbonate, and a flavoring agent.

36. The method of any one of claims 1-35, comprising administering to the subject a formulation of genetically engineered bacterium comprising the genetically engineered bacterium, sucralose, sodium bicarbonate, and a flavoring agent, wherein the amount of genetically engineered bacterium in the formulation is from about 0.5 gram to about 3.5 grams.

37. The method of any one of claims 1-36, comprising administering to the subject a formulation of genetically engineered bacterium comprising the genetically engineered bacterium, sucralose, sodium bicarbonate, and a flavoring agent, wherein the amount of sucralose in the formulation is from about 0.001 grams to about 0.1 grams.

38. The method of any one of claims 1-37, comprising administering to the subject a formulation of genetically engineered bacterium comprising the genetically engineered bacterium, sucralose, sodium bicarbonate, and a flavoring agent, wherein the amount of sodium bicarbonate in the formulation is from about 0.5 gram to about 3.5 grams.

39. The method of any one of claims 1-38, comprising administering to the subject a formulation of genetically engineered bacterium comprising the genetically engineered bacterium, sucralose, sodium bicarbonate, and a flavoring agent, wherein the amount of flavoring agent in the formulation is from about 0.1 grams to about 1 gram.

40. The method of any one of claims 1-39, wherein the subject has phenylketonuria.

41. The method of any one of claims 1-40, wherein the subject has phenylketonuria, classical or typical phenylketonuria, atypical phenylketonuria, mild hyperphenylalaninemia, nonphenylketonuric hyperphenylalaninemia, phenylalanine hydroxylase deficiency, cofactor deficiency, dihydropteridine reductase deficiency, 6-pyruvoyl tetrahydropterin synthase deficiency, Segawa's disease, progressive and irreversible neurological deficits, cognitive impairment, encephalopathy, epilepsy, eczema, reduced growth, microcephaly, tremor, limb spasticity, or hypopigmentation.

42. The method of any one of claims 1-41, wherein the subject has a baseline blood Phe concentration greater of >360 μmol/L at baseline prior to administration of the genetically engineered bacterium or a formulation comprising the genetically engineered bacterium.

43. The method of any one of claims 1-42, wherein the subject has a baseline blood Phe concentration of ≥600 μmol/L at baseline prior to administration of the genetically engineered bacterium a formulation comprising the genetically engineered bacterium.

44. The method of any one of the preceding claims, further comprising administering to the subject an additional blood phenylalanine (Phe) level management regimen.

45. The method of claim 44, wherein the additional blood Phe level management regimen comprises sapropterin or sepiapterin.

46. The method of claim 44 or claim 45, wherein the additional blood Phe level management regimen is administered to the subject at baseline prior to administration of the genetically engineered bacterium or a formulation comprising the genetically engineered bacterium.

47. The method of any one of the preceding claims, wherein the subject is on a Phe-restricted diet.

48. The method of any one of the preceding claims, wherein the subject achieves at least a 20% reduction in the level of phenylalanine after administration of the genetically engineered bacterium, or a formulation comprising the genetically engineered bacterium, as compared to the baseline level of phenylalanine.

49. The method of any one of the preceding claims, wherein the subject achieves at least a 30% reduction in the phenylalanine level after administration of the genetically engineered bacterium, or a formulation comprising the genetically engineered bacterium, as compared to the baseline level of phenylalanine.

50. The method of any one of the preceding claims, wherein the subject achieves at least a 40% reduction in the phenylalanine level after administration of the genetically engineered bacterium, or a formulation comprising the genetically engineered bacterium, as compared to the baseline level of phenylalanine.

51. The method of any one of the preceding claims, wherein the baseline level of phenylalanine in the blood or plasma is ≥600 μmol/L prior to administration of the genetically engineered bacterium, or a formulation comprising the genetically engineered bacterium, and achieves a blood or plasma Phe level≤600 μmol/L after administration of the genetically engineered bacterium, or formulation comprising the genetically engineered bacterium.

52. The method of any one of the preceding claims, wherein the baseline level of phenylalanine in the blood or plasma is ≥600 μmol/L prior to administration of the genetically engineered bacterium, or a formulation comprising the genetically engineered bacterium, and achieves a blood or plasma Phe level≤360 μmol/L after administration of the genetically engineered bacterium, or formulation comprising the genetically engineered bacterium.

53. The method of any one of the preceding claims, wherein the baseline level of Phe in the blood or plasma is ≥600 μmol/L prior to administration of the genetically engineered bacterium, or a formulation comprising the genetically engineered bacterium, and achieves a blood or plasma Phe level≤240 μmol/L after administration of the genetically engineered bacterium, or formulation comprising the genetically engineered bacterium.

54. The method of any one of the preceding claims, wherein the baseline level of Phe in the blood or plasma is >360 μmol/L prior to administration of the genetically engineered bacterium, or a formulation comprising the genetically engineered bacterium, and achieves a blood or plasma Phe level≤360 μmol/L after administration of the genetically engineered bacterium, or formulation comprising the genetically engineered bacterium.

55. The method of any one of the preceding claims, wherein the baseline level of Phe in blood or plasma is >360 μmol/L prior to administration of the genetically engineered bacterium, or a formulation comprising the genetically engineered bacterium, and achieves a blood or plasma Phe level≤240 μmol/L after administration of the genetically engineered bacterium, or formulation comprising the genetically engineered bacterium.

56. The method of any one of the previous claims, further comprising measuring the baseline level of phenylalanine in blood or plasma of the subject, and selecting the subject for treatment when the baseline level is equal to or greater than 600 μmol/L, 360 μmol/L μmol/L or 240 μmol/L.

57. The method of any one of the previous claims, further comprising measuring the phenylalanine level in the blood or plasma of the subject post administration, wherein a decrease of at least 20% as compared to the baseline level indicates that the treatment is effective.

58. The method of any one of the previous claims, comprising:
(i) measuring the baseline level of phenylalanine in blood or plasma of the subject, and selecting the subject for treatment when the baseline level is equal to or greater than 600 μmol/L, 360 μmol/L μmol/L or 240 μmol/L:
(ii) administering the genetically engineered bacterium to the subject for a first dosing interval of 21 days:
a. at a dose of about 1×10¹¹ cells once per day for days 1-3;
b. at a dose of about 1×10¹¹ cells twice per day for days 4-6;
c. at a dose of about 1×10¹¹ cells three times per day for days 7-9;
d. at a dose of about 3×10¹¹ cells once per day for days 10-11;
e. at a dose of about 3×10¹¹ cells twice per day for days 12-14; and
f. at a dose of about 3×10¹¹ cells three times per day for days 15-21,
as determined by live cell counting;
(iii) measuring the phenylalanine level in the blood or plasma of the subject after day 21 of administration;
(iv) determining whether there was at least a 20% reduction in the phenylalanine level in the blood or plasma of the subject after day 21 of administration in step (iii) with the baseline level of phenylalanine in step (i);
(v) selecting the subject for further treatment with the genetically engineered bacterium once at least a 20% reduction in the phenylalanine level in the blood or plasma of the subject after administration as compared to the baseline level is achieved, and
(vi) optionally further treating the patient with the genetically engineered bacterium.

59. The method of claim 58, further comprising:
(vii) administering the genetically engineered bacterium to the subject for a second dosing interval of 21 days at a dose of about 6×10¹¹ cells, as determined by live cell counting:
a. once per day for days 1-3;
b. twice per day for days 4-7; and
c. three times per day for days 8-21;
(viii) measuring the phenylalanine level in the blood or plasma of the subject after day 21 of administration;
(ix) determining whether there was at least a 20% reduction in the phenylalanine level in the blood or plasma of the subject after day 21 of administration in step (vii) with the baseline level of phenylalanine in step (i);
(x) selecting the subject for further treatment with the genetically engineered bacterium once at least a 20% reduction in the phenylalanine level in the blood or plasma of the subject after administration as compared to the baseline level is achieved, and
(xi) optionally further treating the patient with the genetically engineered bacterium.

60. The method of claim 59, further comprising:
(xii) administering the genetically engineered bacterium to the subject for a third dosing interval of at least 21 days in length at a dose of about 1×10¹² cells, as determined by live cell counting:
a. once per day for days 1-3;
b. twice per day for days 4-7; and
c. three times per day for at least days 8-21;
(xiii) measuring the phenylalanine level in the blood or plasma of the subject after day 21 of administration;
(xiv) determining whether there was at least a 20% reduction in the phenylalanine level in the blood or plasma of the subject after day 21 of administration in step (xiii) with the baseline level of phenylalanine in step (i);
(xv) selecting the subject for further treatment with the genetically engineered bacterium once at least a 20% reduction in the phenylalanine level in the blood or plasma of the subject after administration as compared to the baseline level is achieved, and
(xvi) optionally further treating the patient with the genetically engineered bacterium.

61. The method of any one of claims 58-60, selecting the subject for further treatment with the genetically engineered bacterium once at least a 25% or at least a 30% reduction in the phenylalanine level in the blood or plasma of the subject after administration as compared to the baseline level is achieved.


SEQUENCE LISTING (510 sequences)

SEQ ID NO: 1 - DNA, 290 bp, synthetic construct
  misc_feature 1..290: note = Synthetic: FNR Responsive Promoter
  source 1..290: mol_type = other DNA
gtcagcataacaccctgacctctcattaattgttcatgccgggcggcactatcgtcgtccggccttttcctctcttactctgctacgtacatctatttctataaatccgttcaatttgtctgttttttgcacaaacatgaaatatcagacaattccgtgacttaagaaaatttatacaaatcagcaatataccccttaaggagtatataaaggtgaatttgatttacatcaataagcggggttgctgaatcgttaaggtaggcggtaatagaaaagaaatcgaggcaaaa

SEQ ID NO: 2 - DNA, 173 bp, synthetic construct
  misc_feature 1..173: note = Synthetic: FNR Responsive Promoter
  source 1..173: mol_type = other DNA
atttcctctcatcccatccggggtgagagtcttttcccccgacttatggctcatgcatgcatcaaaaaagatgtgagcttgatcaaaaacaaaaaatatttcactcgacaggagtatttatattgcgcccgttacgtgggcttcgactgtaaatcagaaaggagaaaacacct

SEQ ID NO: 3 - DNA, 305 bp, synthetic construct
  misc_feature 1..305: note = Synthetic: FNR Responsive Promoter
  source 1..305: mol_type = other DNA
gtcagcataacaccctgacctctcattaattgttcatgccgggcggcactatcgtcgtccggccttttcctctcttactctgctacgtacatctatttctataaatccgttcaatttgtctgttttttgcacaaacatgaaatatcagacaattccgtgacttaagaaaatttatacaaatcagcaatataccccttaaggagtatataaaggtgaatttgatttacatcaataagcggggttgctgaatcgttaaggatccctctagaaataattttgtttaactttaagaaggagatatacat

SEQ ID NO: 4 - DNA, 180 bp, synthetic construct
  misc_feature 1..180: note = Synthetic: FNR Responsive Promoter
  source 1..180: mol_type = other DNA
catttcctctcatcccatccggggtgagagtcttttcccccgacttatggctcatgcatgcatcaaaaaagatgtgagcttgatcaaaaacaaaaaatatttcactcgacaggagtatttatattgcgcccggatccctctagaaataattttgtttaactttaagaaggagatatacat

SEQ ID NO: 5 - DNA, 199 bp, synthetic construct
  misc_feature 1..199: note = Synthetic: FNR Responsive Promoter
  source 1..199: mol_type = other DNA
agttgttcttattggtggtgttgctttatggttgcatcgtagtaaatggttgtaacaaaagcaatttttccggctgtctgtatacaaaaacgccgtaaagtttgagcgaagtcaataaactctctacccattcagggcaatatctctcttggatccctctagaaataattttgtttaactttaagaaggagatatacat

SEQ ID NO: 6 - DNA, 117 bp, synthetic construct
  misc_feature 1..117: note = Synthetic: FNR-responsive regulatory region sequence
  source 1..117: mol_type = other DNA
atccccatcactcttgatggagatcaattccccaagctgctagagcgttaccttgcccttaaacattagcaatgtcgatttatcagagggccgacaggctcccacaggagaaaaccg

SEQ ID NO: 7 - DNA, 108 bp, synthetic construct
  misc_feature 1..108: note = Synthetic: FNR-responsive regulatory region sequence
  source 1..108: mol_type = other DNA
ctcttgatcgttatcaattcccacgctgtttcagagcgttaccttgcccttaaacattagcaatgtcgatttatcagagggccgacaggctcccacaggagaaaaccg

SEQ ID NO: 8 - DNA, 433 bp, synthetic construct
  misc_feature 1..433: note = Synthetic: FNR-responsive regulatory region, nirB2
  source 1..433: mol_type = other DNA
cggcccgatcgttgaacatagcggtccgcaggcggcactgcttacagcaaacggtctgtacgctgtcgtctttgtgatgtgcttcctgttaggtttcgtcagccgtcaccgtcagcataacaccctgacctctcattaattgctcatgccggacggcactatcgtcgtccggccttttcctctcttcccccgctacgtgcatctatttctataaacccgctcattttgtctattttttgcacaaacatgaaatatcagacaattccgtgacttaagaaaatttatacaaatcagcaatatacccattaaggagtatataaaggtgaatttgatttacatcaataagcggggttgctgaatcgttaaggtaggcggtaatagaaaagaaatcgaggcaaaaatgtttgtttaactttaagaaggagatatacat

SEQ ID NO: 9 - DNA, 290 bp, synthetic construct
  misc_feature 1..290: note = Synthetic: FNR-responsive regulatory region, nirB3
  source 1..290: mol_type = other DNA
gtcagcataacaccctgacctctcattaattgctcatgccggacggcactatcgtcgtccggccttttcctctcttcccccgctacgtgcatctatttctataaacccgctcattttgtctattttttgcacaaacatgaaatatcagacaattccgtgacttaagaaaatttatacaaatcagcaatatacccattaaggagtatataaaggtgaatttgatttacatcaataagcggggttgctgaatcgttaaggtaggcggtaatagaaaagaaatcgaggcaaaa

SEQ ID NO: 10 - DNA, 207 bp, synthetic construct
  misc_feature 1..207: note = Synthetic: FNR-responsive regulatory region, fnrS2
  source 1..207: mol_type = other DNA
agttgttcttattggtggtgttgctttatggttgcatcgtagtaaatggttgtaacaaaagcaatttttccggctgtctgtatacaaaaacgccgcaaagtttgagcgaagtcaataaactctctacccattcagggcaatatctctcttggatccaaagtgaactctagaaataattttgtttaactttaagaaggagatatacat

SEQ ID NO: 11 - DNA, 390 bp, synthetic construct
  misc_feature 1..390: note = Synthetic: FNR-responsive regulatory region, Pfnr6 nirB+crp
  source 1..390: mol_type = other DNA
tcgtctttgtgatgtgcttcctgttaggtttcgtcagccgtcaccgtcagcataacaccctgacctctcattaattgctcatgccggacggcactatcgtcgtccggccttttcctctcttcccccgctacgtgcatctatttctataaacccgctcattttgtctattttttgcacaaacatgaaatatcagacaattccgtgacttaagaaaatttatacaaatcagcaatatacccattaaggagtatataaaggtgaatttgatttacatcaataagcggggttgctgaatcgttaaggtagaaatgtgatctagttcacatttgcggtaatagaaaagaaatcgaggcaaaaatgtttgtttaactttaagaaggagatatacat

SEQ ID NO: 12 - DNA, 200 bp, synthetic construct
  misc_feature 1..200: note = Synthetic: FNR-responsive regulatory region, Pfnr7 fnrS+crp
  source 1..200: mol_type = other DNA
agttgttcttattggtggtgttgctttatggttgcatcgtagtaaatggttgtaacaaaagcaatttttccggctgtctgtatacaaaaacgccgcaaagtttgagcgaagtcaataaactctctacccattcagggcaatatctctcaaatgtgatctagttcacattttttgtttaactttaagaaggagatatacat

SEQ ID NO: 13 - DNA, 164 bp, synthetic construct
  misc_feature 1..164: note = Synthetic: FNR-responsive promoter
  source 1..164: mol_type = other DNA
agttgttcttattggtggtgttgctttatggttgcatcgtagtaaatggttgtaacaaaagcaatttttccggctgtctgtatacaaaaacgccgcaaagtttgagcgaagtcaataaactctctacccattcagggcaatatctctcttggatccaaagtgaa

SEQ ID NO: 14 - DNA, 201 bp, synthetic construct
  misc_feature 1..201: note = Synthetic: FNR-responsive promoter
  source 1..201: mol_type = other DNA
agttgttcttattggtggtgttgctttatggttgcatcgtagtaaatggttgtaacaaaagcaatttttccggctgtctgtatacaaaaacgccgcaaagtttgagcgaagtcaataaactctctacccattcagggcaatatctctcttggatccaaagtgaacccgcatcccacgctaactaatataaggggggcaaga

SEQ ID NO: 15 - DNA, 1083 bp, synthetic construct
  misc_feature 1..1083: note = Synthetic: LacI in reverse orientation
  source 1..1083: mol_type = other DNA
tcactgcccgctttccagtcgggaaacctgtcgtgccagctgcattaatgaatcggccaacgcgcggggagaggcggtttgcgtattgggcgccagggtggtttttcttttcaccagtgagactggcaacagctgattgcccttcaccgcctggccctgagagagttgcagcaagcggtccacgctggtttgccccagcaggcgaaaatcctgtttgatggtggttaacggcgggatataacatgagctatcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcctcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcat

SEQ ID NO: 16 - protein, 360 aa, synthetic construct
  REGION 1..360: note = Synthetic: LacI
MKPVTLYDVAEYAGVSYQTVSRVVNQASHVSAKTREKVEAAMAELNYIPNRVAQQLAGKQSLLIGVATSSLALHAPSQIVAAIKSRADQLGASVVVSMVERSGVEACKAAVHNLLAQRVSGLIINYPLDDQDAIAVEAACTNVPALFLDVSDQTPINSIIFSHEDGTRLGVEHLVALGHQQIALLAGPLSSVSARLRLAGWHKYLTRNQIQPIAEREGDWSAMSGFQQTMQMLNEGIVPTAMLVANDQMALGAMRAITESGLRVGADISVVGYDDTEDSSCYIPPLTTIKQDFRLLGQTSVDRLLQLSQGQAVKGNQLLPVSLVKRKTTLAPNTQTASPRALADSLMQLARQVSRLESGQ

SEQ ID NO: 17 - DNA, 20 bp, synthetic construct
  misc_feature 1..20: note = Synthetic: Lac operator
  source 1..20: mol_type = other DNA
aattgtgagcgctcacaatt

SEQ ID NO: 18 - DNA, 263 bp, synthetic construct
  misc_feature 1..263: note = Synthetic: Promoter comprising Lac operon
  source 1..263: mol_type = other DNA
attcaccaccctgaattgactctcttccgggcgctatcatgccataccgcgaaaggttttgcgccattcgatggcgcgccgcttcgtcaggccacatagctttcttgttctgatcggaacgatcgttggctgtgttgacaattaatcatcggctcgtataatgtgtggaattgtgagcgctcacaattagctgtcaccggatgtgctttccggtctgatgagtccgtgaggacgaaacagcctctacaaataattttgtttaa

SEQ ID NO: 19 - DNA, 222 bp, synthetic construct
  misc_feature 1..222: note = Synthetic: Description of Artificial Sequence: Synthetic polynucleotide
  source 1..222: mol_type = other DNA
acgttaaatctatcaccgcaagggataaatatctaacaccgtgcgtgttgactattttacctctggcggtgataatggttgcatagctgtcaccggatgtgctttccggtctgatgagtccgtgaggacgaaacagcctctacaaataattttgtttaaaacaacacccactaagataactctagaaataattttgtttaactttaagaaggagatatacat

SEQ ID NO: 20 - DNA, 714 bp, synthetic construct
  misc_feature 1..714: note = Synthetic: Description of Artificial Sequence: Synthetic polynucleotide
  source 1..714: mol_type = other DNA
tcagccaaacgtctcttcaggccactgactagcgataactttccccacaacggaacaactctcattgcatgggatcattgggtactgtgggtttagtggttgtaaaaacacctgaccgctatccctgatcagtttcttgaaggtaaactcatcacccccaagtctggctatgcagaaatcacctggctcaacagcctgctcagggtcaacgagaattaacattccgtcaggaaagcttggcttggagcctgttggtgcggtcatggaattaccttcaacctcaagccagaatgcagaatcactggcttttttggttgtgcttacccatctctccgcatcacctttggtaaaggttctaagcttaggtgagaacatccctgcctgaacatgagaaaaaacagggtactcatactcacttctaagtgacggctgcatactaaccgcttcatacatctcgtagatttctctggcgattgaagggctaaattcttcaacgctaactttgagaatttttgtaagcaatgcggcgttataagcatttaatgcattgatgccattaaataaagcaccaacgcctgactgccccatccccatcttgtctgcgacagattcctgggataagccaagttcatttttctttttttcataaattgctttaaggcgacgtgcgtcctcaagctgctcttgtgttaatggtttcttttttgtgctcat

SEQ ID NO: 21 - protein, 237 aa, synthetic construct
  REGION 1..237: note = Synthetic: Description of Artificial Sequence: Synthetic polypeptide
MSTKKKPLTQEQLEDARRLKAIYEKKKNELGLSQESVADKMGMGQSGVGALFNGINALNAYNAALLTKILKVSVEEFSPSIAREIYEMYEAVSMQPSLRSEYEYPVFSHVQAGMFSPKLRTFTKGDAERWVSTTKKASDSAFWLEVEGNSMTAPTGSKPSFPDGMLILVDPEQAVEPGDFCIARLGGDEFTFKKLIRDSGQVFLQPLNPQYPMIPCNESCSVVGKVIASQWPEETFG

SEQ ID NO: 22 - DNA, 84 bp, synthetic construct
  misc_feature 1..84: note = Synthetic: Description of Artificial Sequence: Synthetic oligonucleotide
  source 1..84: mol_type = other DNA
acgttaaatctatcaccgcaagggataaatatctaacaccgtgcgtgttgactattttacctctggcggtgataatggttgcat

SEQ ID NO: 23 - DNA, 714 bp, synthetic construct
  misc_feature 1..714: note = Synthetic: Description of Artificial Sequence: Synthetic polynucleotide
  source 1..714: mol_type = other DNA
atgagcacaaaaaagaaaccattaacacaagagcagcttgaggacgcacgtcgccttaaagcaatttatgaaaaaaagaaaaatgaacttggcttatcccaggaatctgtcgcagacaagatggggatggggcagtcaggcgttggtgccttatttaatggcatcaatgcattaaatgcttataacgccgcatcgcttacaagaattctcaaagttagcgttgaagaatttagcccttcaatcgccagagaaatctacgagatgtatgaagcggttagtatgcagccgtcacttagaagtgagtatgagtaccctgttttttctcatgttcaggcagggatgctctcacctgagcttagaacctttaccaaaggtggtgcggaaaggtgggtaagcacaaccaaaaaagccagtgattctgcattctggcttgaggttgaaggtaattccatgacagcaccaacaggctccaagccaagctttcctgacggaatgttaattctcgttgaccctgagcaggctgttgagccaggtgatttctgcatagccagactcgggggtggtgagtttaccttcaagaaactgatcagggatagcggtcaggtgtttttacaaccactaaacccacagtacccaatgatcccatgcaatgagagttgttccgttgtggggaaagttatcgctagtcagtggcctgaagagacgtttggctga

SEQ ID NO: 24 - protein, 237 aa, synthetic construct
  REGION 1..237: note = Synthetic: Description of Artificial Sequence: Synthetic polypeptide
MSTKKKPLTQEQLEDARRLKAIYEKKKNELGLSQESVADKMGMGQSGVGALFNGINALNAYNAASLTRILKVSVEEFSPSIAREIYEMYEAVSMQPSLRSEYEYPVFSHVQAGMLSPELRTFTKGGAERWVSTTKKASDSAFWLEVEGNSMTAPTGSKPSFPDGMLILVDPEQAVEPGDFCIARLGGGEFTFKKLIRDSGQVFLQPLNPQYPMIPCNESCSVVGKVIASQWPEETFG

SEQ ID NO: 25 - DNA, 183 bp, synthetic construct
  misc_feature 1..183: note = Synthetic: Description of Artificial Sequence: Synthetic polynucleotide
  source 1..183: mol_type = other DNA
aaatctatcaccgcaagggataaatatctaacaccgtgcgtgttgactattttacctctggcggtgataatggttgcatagctgtcaccggatgtgctttccggtctgatgagtccgtgaggacgaaacagcctctacaaataattttgtttaaaacaacacccactaagataaggtagaaac

SEQ ID NO: 26 - DNA, 56 bp, synthetic construct
  misc_feature 1..56: note = Synthetic: ptet promoter
  source 1..56: mol_type = other DNA
gttgacactctatcattgatagagttattttaccactccctatcagtgatagagaa

SEQ ID NO: 27 - DNA, 123 bp, synthetic construct
  misc_feature 1..123: note = Synthetic: native cumate inducible promoter
  source 1..123: mol_type = other DNA
taacaccgtgcgtgttgactattttacctctggcggtgataatggttgcaacaaacagacaatctggtctgtttgtattatgttaaccagcacactggcggccgcttactagaaataattttg

SEQ ID NO: 28 - DNA, 161 bp, synthetic construct
  misc_feature 1..161: note = Synthetic: native cumate inducible promoter
  source 1..161: mol_type = other DNA
taacaccgtgcgtgttgactattttacctctggcggtgataatggttgcaacaaacagacaatctggtctgtttgtattatgttaaccagcacactggcggccgcttactagaaataattttggccgcatcccacgctaactaatataaaggaggttttaa

SEQ ID NO: 29 - DNA, 79 bp, synthetic construct
  misc_feature 1..79: note = Synthetic: native salicylate inducible promoter
  source 1..79: mol_type = other DNA
tgtatttatcaatattgtttgctccgttatcgttattaacaagtcatcaataaagccatcacgagtaccatagaggatc

SEQ ID NO: 30 - DNA, 117 bp, synthetic construct
  misc_feature 1..117: note = Synthetic: native salicylate inducible promoter
  source 1..117: mol_type = other DNA
tgtatttatcaatattgtttgctccgttatcgttattaacaagtcatcaataaagccatcacgagtaccatagaggatcgccgcatcccacgctaactaatataaaggaggttttaa

SEQ ID NO: 31 - DNA, 42 bp, synthetic construct
  misc_feature 1..42: note = Synthetic: apFAB124 promoter
  source 1..42: mol_type = other DNA
tcgacatttatcccttgcggcgaatacttacagccatagcaa

SEQ ID NO: 32 - DNA, 49 bp, synthetic construct
  misc_feature 1..49: note = Synthetic: apfab338 promtoer
  source 1..49: mol_type = other DNA
ggcgcgccttgacaattaatcatccggctcctaggatgtgtggagggac

SEQ ID NO: 33 - DNA, 50 bp, synthetic construct
  misc_feature 1..50: note = Synthetic: apFAB66 promoter
  source 1..50: mol_type = other DNA
ggcgcgccttgacatcaggaaaatttttctgtataatagattcatctcaa

SEQ ID NO: 34 - DNA, 51 bp, synthetic construct
  misc_feature 1..51: note = Synthetic: apFAB54 promoter
  source 1..51: mol_type = other DNA
ggcgcgccttgacataaagtctaacctataggatacttacagccatacaag

SEQ ID NO: 35 - DNA, 66 bp, synthetic construct
  misc_feature 1..66: note = Synthetic: Lpp promoter from E. coli
  source 1..66: mol_type = other DNA
ataagtgccttcccatcaaaaaaatattctcaacataaaaaactttgtgtaatacttgtaacgcta

SEQ ID NO: 36 - DNA, 96 bp, synthetic construct
  misc_feature 1..96: note = Synthetic: Lpp promoter from E. coli
  source 1..96: mol_type = other DNA
ataagtgccttcccatcaaaaaaatattctcaacataaaaaactttgtgtaatacttgtaacgctagtgaattatataaaagtgggaggtgcccga

SEQ ID NO: 37 - DNA, 30 bp, synthetic construct
  misc_feature 1..30: note = Synthetic: Description of Artificial Sequence: Synthetic polynucleotide
  source 1..30: mol_type = other DNA
gtttatacataggcgagtactctgttatgg

SEQ ID NO: 38 - DNA, 30 bp, synthetic construct
  misc_feature 1..30: note = Synthetic: Description of Artificial Sequence: Synthetic polynucleotide
  source 1..30: mol_type = other DNA
agaggttccaactttcaccataatgaaaca

SEQ ID NO: 39 - DNA, 30 bp, synthetic construct
  misc_feature 1..30: note = Synthetic: Description of Artificial Sequence: Synthetic polynucleotide
  source 1..30: mol_type = other DNA
taaacaactaacggacaattctacctaaca

SEQ ID NO: 40 - DNA, 30 bp, synthetic construct
  misc_feature 1..30: note = Synthetic: Description of Artificial Sequence: Synthetic polynucleotide
  source 1..30: mol_type = other DNA
acatcaagccaaattaaacaggattaacac

SEQ ID NO: 41 - DNA, 30 bp, synthetic construct
  misc_feature 1..30: note = Synthetic: Description of Artificial Sequence: Synthetic polynucleotide
  source 1..30: mol_type = other DNA
gaggtaaaatagtcaacacgcacggtgtta

SEQ ID NO: 42 - DNA, 30 bp, synthetic construct
  misc_feature 1..30: note = Synthetic: Description of Artificial Sequence: Synthetic polynucleotide
  source 1..30: mol_type = other DNA
caggccggaataactccctataatgcgcca

SEQ ID NO: 43 - DNA, 30 bp, synthetic construct
  misc_feature 1..30: note = Synthetic: Description of Artificial Sequence: Synthetic polynucleotide
  source 1..30: mol_type = other DNA
ggctagctcagtcctaggtacagtgctagc

SEQ ID NO: 44 - DNA, 30 bp, synthetic construct
  misc_feature 1..30: note = Synthetic: Description of Artificial Sequence: Synthetic polynucleotide
  source 1..30: mol_type = other DNA
agctagctcagtcctaggtattatgctagc

SEQ ID NO: 45 - DNA, 30 bp, synthetic construct
  misc_feature 1..30: note = Synthetic: Description of Artificial Sequence: Synthetic polynucleotide
  source 1..30: mol_type = other DNA
agctagctcagtcctaggtactgtgctagc

SEQ ID NO: 46 - DNA, 30 bp, synthetic construct
  misc_feature 1..30: note = Synthetic: Description of Artificial Sequence: Synthetic polynucleotide
  source 1..30: mol_type = other DNA
agctagctcagtcctagggattatgctagc

SEQ ID NO: 47 - DNA, 30 bp, synthetic construct
  misc_feature 1..30: note = Synthetic: Description of Artificial Sequence: Synthetic polynucleotide
  source 1..30: mol_type = other DNA
agctagctcagtcctaggtattgtgctagc

SEQ ID NO: 48 - DNA, 30 bp, synthetic construct
  misc_feature 1..30: note = Synthetic: Description of Artificial Sequence: Synthetic polynucleotide
  source 1..30: mol_type = other DNA
ggctagctcagtcctaggtactatgctagc

SEQ ID NO: 49 - DNA, 30 bp, synthetic construct
  misc_feature 1..30: note = Synthetic: Description of Artificial Sequence: Synthetic polynucleotide
  source 1..30: mol_type = other DNA
ggctagctcagtcctaggtatagtgctagc

SEQ ID NO: 50 - DNA, 30 bp, synthetic construct
  misc_feature 1..30: note = Synthetic: Description of Artificial Sequence: Synthetic polynucleotide
  source 1..30: mol_type = other DNA
ggctagctcagccctaggtattatgctagc

SEQ ID NO: 51 - DNA, 30 bp, synthetic construct
  misc_feature 1..30: note = Synthetic: Description of Artificial Sequence: Synthetic polynucleotide
  source 1..30: mol_type = other DNA
agctagctcagtcctaggtataatgctagc

SEQ ID NO: 52 - DNA, 30 bp, synthetic construct
  misc_feature 1..30: note = Synthetic: Description of Artificial Sequence: Synthetic polynucleotide
  source 1..30: mol_type = other DNA
agctagctcagtcctagggactgtgctagc

SEQ ID NO: 53 - DNA, 30 bp, synthetic construct
  misc_feature 1..30: note = Synthetic: Description of Artificial Sequence: Synthetic polynucleotide
  source 1..30: mol_type = other DNA
ggctagctcagtcctaggtacaatgctagc

SEQ ID NO: 54 - DNA, 30 bp, synthetic construct
  misc_feature 1..30: note = Synthetic: Description of Artificial Sequence: Synthetic polynucleotide
  source 1..30: mol_type = other DNA
ggctagctcagtcctagggattatgctagc

SEQ ID NO: 55 - DNA, 30 bp, synthetic construct
  misc_feature 1..30: note = Synthetic: Description of Artificial Sequence: Synthetic polynucleotide
  source 1..30: mol_type = other DNA
agctagctcagcccttggtacaatgctagc

SEQ ID NO: 56 - DNA, 30 bp, synthetic construct
  misc_feature 1..30: note = Synthetic: Description of Artificial Sequence: Synthetic polynucleotide
  source 1..30: mol_type = other DNA
agctagctcagtcctagggactatgctagc

SEQ ID NO: 57 - DNA, 30 bp, synthetic construct
  misc_feature 1..30: note = Synthetic: Description of Artificial Sequence: Synthetic polynucleotide
  source 1..30: mol_type = other DNA
agctagctcagtcctagggattgtgctagc

SEQ ID NO: 58 - DNA, 30 bp, synthetic construct
  misc_feature 1..30: note = Synthetic: Description of Artificial Sequence: Synthetic polynucleotide
  source 1..30: mol_type = other DNA
ggctagctcagtcctaggtattgtgctagc

SEQ ID NO: 59 - DNA, 30 bp, synthetic construct
  misc_feature 1..30: note = Synthetic: Description of Artificial Sequence: Synthetic polynucleotide
  source 1..30: mol_type = other DNA
ggctagctcagtcctaggtattatgctagc

SEQ ID NO: 60 - DNA, 30 bp, synthetic construct
  misc_feature 1..30: note = Synthetic: Description of Artificial Sequence: Synthetic polynucleotide
  source 1..30: mol_type = other DNA
aaagtgtgacgccgtgcaaataatcaatgt

SEQ ID NO: 61 - DNA, 30 bp, synthetic construct
  misc_feature 1..30: note = Synthetic: Description of Artificial Sequence: Synthetic polynucleotide
  source 1..30: mol_type = other DNA
gacgaatacttaaaatcgtcatacttattt

SEQ ID NO: 62 - DNA, 30 bp, synthetic construct
  misc_feature 1..30: note = Synthetic: Description of Artificial Sequence: Synthetic polynucleotide
  source 1..30: mol_type = other DNA
aaacctttcgcggtatggcatgatagcgcc

SEQ ID NO: 63 - DNA, 30 bp, synthetic construct
  misc_feature 1..30: note = Synthetic: Description of Artificial Sequence: Synthetic polynucleotide
  source 1..30: mol_type = other DNA
tgatagcgcccggaagagagtcaattcagg

SEQ ID NO: 64 - DNA, 30 bp, synthetic construct
  misc_feature 1..30: note = Synthetic: Description of Artificial Sequence: Synthetic polynucleotide
  source 1..30: mol_type = other DNA
ttatttaccgtgacgaactaattgctcgtg

SEQ ID NO: 65 - DNA, 30 bp, synthetic construct
  misc_feature 1..30: note = Synthetic: Description of Artificial Sequence: Synthetic polynucleotide
  source 1..30: mol_type = other DNA
catacgccgttatacgttgtttacgctttg

SEQ ID NO: 66 - DNA, 30 bp, synthetic construct
  misc_feature 1..30: note = Synthetic: Description of Artificial Sequence: Synthetic polynucleotide
  source 1..30: mol_type = other DNA
ttatgcttccggctcgtatgttgtgtggac

SEQ ID NO: 67 - DNA, 30 bp, synthetic construct
  misc_feature 1..30: note = Synthetic: Description of Artificial Sequence: Synthetic polynucleotide
  source 1..30: mol_type = other DNA
ttatgcttccggctcgtatggtgtgtggac

SEQ ID NO: 68 - DNA, 30 bp, synthetic construct
  misc_feature 1..30: note = Synthetic: Description of Artificial Sequence: Synthetic polynucleotide
  source 1..30: mol_type = other DNA
atatatatatatatataatggaagcgtttt

SEQ ID NO: 69 - DNA, 30 bp, synthetic construct
  misc_feature 1..30: note = Synthetic: Description of Artificial Sequence: Synthetic polynucleotide
  source 1..30: mol_type = other DNA
ccccgaaagcttaagaatataattgtaagc

SEQ ID NO: 70 - DNA, 30 bp, synthetic construct
  misc_feature 1..30: note = Synthetic: Description of Artificial Sequence: Synthetic polynucleotide
  source 1..30: mol_type = other DNA
tgacaatatatatatatatataatgctagc

SEQ ID NO: 71 - DNA, 30 bp, synthetic construct
  misc_feature 1..30: note = Synthetic: Description of Artificial Sequence: Synthetic polynucleotide
  source 1..30: mol_type = other DNA
acaatatatatatatatatataatgctagc

SEQ ID NO: 72 - DNA, 30 bp, synthetic construct
  misc_feature 1..30: note = Synthetic: Description of Artificial Sequence: Synthetic polynucleotide
  source 1..30: mol_type = other DNA
aatatatatatatatatatataatgctagc

SEQ ID NO: 73 - DNA, 30 bp, synthetic construct
  misc_feature 1..30: note = Synthetic: Description of Artificial Sequence: Synthetic polynucleotide
  source 1..30: mol_type = other DNA
tatatatatatatatatatataatgctagc

SEQ ID NO: 74 - DNA, 30 bp, synthetic construct
  misc_feature 1..30: note = Synthetic: Description of Artificial Sequence: Synthetic polynucleotide
  source 1..30: mol_type = other DNA
aaaaaaaaaaaaaaaaaatataatgctagc

SEQ ID NO: 75 - DNA, 30 bp, synthetic construct
  misc_feature 1..30: note = Synthetic: Description of Artificial Sequence: Synthetic polynucleotide
  source 1..30: mol_type = other DNA
ggaattgtgagcggataacaatttcacaca

SEQ ID NO: 76 - DNA, 30 bp, synthetic construct
  misc_feature 1..30: note = Synthetic: Description of Artificial Sequence: Synthetic polynucleotide
  source 1..30: mol_type = other DNA
gattaaagaggagaaatactagagtactag

SEQ ID NO: 77 - DNA, 30 bp, synthetic construct
  misc_feature 1..30: note = Synthetic: Description of Artificial Sequence: Synthetic polynucleotide
  source 1..30: mol_type = other DNA
caccttcgggtgggcctttctgcgtttata

SEQ ID NO: 78 - DNA, 30 bp, synthetic construct
  misc_feature 1..30: note = Synthetic: Description of Artificial Sequence: Synthetic polynucleotide
  source 1..30: mol_type = other DNA
tgctagctactagagattaaagaggagaaa

SEQ ID NO: 79 - DNA, 30 bp, synthetic construct
  misc_feature 1..30: note = Synthetic: Description of Artificial Sequence: Synthetic polynucleotide
  source 1..30: mol_type = other DNA
ttgtgagcggataacaagatactgagcaca

SEQ ID NO: 80 - DNA, 30 bp, synthetic construct
  misc_feature 1..30: note = Synthetic: Description of Artificial Sequence: Synthetic polynucleotide
  source 1..30: mol_type = other DNA
cctgtttttatgttattctctctgtaaagg

SEQ ID NO: 81 - DNA, 30 bp, synthetic construct
  misc_feature 1..30: note = Synthetic: Description of Artificial Sequence: Synthetic polynucleotide
  source 1..30: mol_type = other DNA
aaatatttgcttatacaatcttcctgtttt

SEQ ID NO: 82 - DNA, 30 bp, synthetic construct
  misc_feature 1..30: note = Synthetic: Description of Artificial Sequence: Synthetic polynucleotide
  source 1..30: mol_type = other DNA
gctgataaaccgatacaattaaaggctcct

SEQ ID NO: 83 - DNA, 30 bp, synthetic construct
  misc_feature 1..30: note = Synthetic: Description of Artificial Sequence: Synthetic polynucleotide
  source 1..30: mol_type = other DNA
ctcttctcagcgtcttaatctaagctatcg

SEQ ID NO: 84 - DNA, 30 bp, synthetic construct
  misc_feature 1..30: note = Synthetic: Description of Artificial Sequence: Synthetic polynucleotide
  source 1..30: mol_type = other DNA
atgagccagttcttaaaatcgcataaggta

SEQ ID NO: 85 - DNA, 30 bp, synthetic construct
  misc_feature 1..30: note = Synthetic: Description of Artificial Sequence: Synthetic polynucleotide
  source 1..30: mol_type = other DNA
ctattgattgtgacaaaataaacttattcc

SEQ ID NO: 86 - DNA, 30 bp, synthetic construct
  misc_feature 1..30: note = Synthetic: Description of Artificial Sequence: Synthetic polynucleotide
  source 1..30: mol_type = other DNA
gtttcgcgcttggtataatcgctgggggtc

SEQ ID NO: 87 - DNA, 30 bp, synthetic construct
  misc_feature 1..30: note = Synthetic: Description of Artificial Sequence: Synthetic polynucleotide
  source 1..30: mol_type = other DNA
ctttgcttctgactataatagtcagggtaa

SEQ ID NO: 88 - DNA, 30 bp, synthetic construct
  misc_feature 1..30: note = Synthetic: Description of Artificial Sequence: Synthetic polynucleotide
  source 1..30: mol_type = other DNA
aaaccgatacaattaaaggctcctgctagc

SEQ ID NO: 89 - DNA, 30 bp, synthetic construct
  misc_feature 1..30: note = Synthetic: Description of Artificial Sequence: Synthetic polynucleotide
  source 1..30: mol_type = other DNA
caccacactgatagtgctagtgtagatcac

SEQ ID NO: 90 - DNA, 30 bp, synthetic construct
  misc_feature 1..30: note = Synthetic: Description of Artificial Sequence: Synthetic polynucleotide
  source 1..30: mol_type = other DNA
gccggaataactccctataatgcgccacca

SEQ ID NO: 91 - DNA, 30 bp, synthetic construct
  misc_feature 1..30: note = Synthetic: Description of Artificial Sequence: Synthetic polynucleotide
  source 1..30: mol_type = other DNA
ttgacaagcttttcctcagctccgtaaact

SEQ ID NO: 92 - DNA, 30 bp, synthetic construct
  misc_feature 1..30: note = Synthetic: Description of Artificial Sequence: Synthetic polynucleotide
  source 1..30: mol_type = other DNA
ggtttcaaaattgtgatctatatttaacaa

SEQ ID NO: 93 - DNA, 30 bp, synthetic construct
  misc_feature 1..30: note = Synthetic: Description of Artificial Sequence: Synthetic polynucleotide
  source 1..30: mol_type = other DNA
tctattccaataaagaaatcttcctgcgtg

SEQ ID NO: 94 - DNA, 30 bp, synthetic construct
  misc_feature 1..30: note = Synthetic: Description of Artificial Sequence: Synthetic polynucleotide
  source 1..30: mol_type = other DNA
gaccgaatatatagtggaaacgtttagatg

SEQ ID NO: 95 - DNA, 30 bp, synthetic construct
  misc_feature 1..30: note = Synthetic: Description of Artificial Sequence: Synthetic polynucleotide
  source 1..30: mol_type = other DNA
ccacatcctgtttttaaccttaaaatggca

SEQ ID NO: 96 - DNA, 30 bp, synthetic construct
  misc_feature 1..30: note = Synthetic: Description of Artificial Sequence: Synthetic polynucleotide
  source 1..30: mol_type = other DNA
aaaaatgggctcgtgttgtacaataaatgt

SEQ ID NO: 97 - DNA, 30 bp, synthetic construct
  misc_feature 1..30: note = Synthetic: Description of Artificial Sequence: Synthetic polynucleotide
  source 1..30: mol_type = other DNA
aaaaaaagcgcgcgattatgtaaaatataa

SEQ ID NO: 98 - DNA, 30 bp, synthetic construct
  misc_feature 1..30: note = Synthetic: Description of Artificial Sequence: Synthetic polynucleotide
  source 1..30: mol_type = other DNA
aattgcagtaggcatgacaaaatggactca

SEQ ID NO: 99 - DNA, 30 bp, synthetic construct
  misc_feature 1..30: note = Synthetic: Description of Artificial Sequence: Synthetic polynucleotide
  source 1..30: mol_type = other DNA
caagcttttcctttataatagaatgaatga

SEQ ID NO: 100 - DNA, 30 bp, synthetic construct
  misc_feature 1..30: note = Synthetic: Description of Artificial Sequence: Synthetic polynucleotide
  source 1..30: mol_type = other DNA
tctaagctagtgtattttgcgtttaatagt

SEQ ID NO: 101 - DNA, 30 bp, synthetic construct
  misc_feature 1..30: note = Synthetic: Description of Artificial Sequence: Synthetic polynucleotide
  source 1..30: mol_type = other DNA
aatgggctcgtgttgtacaataaatgtagt

SEQ ID NO: 102 - DNA, 30 bp, synthetic construct
  misc_feature 1..30: note = Synthetic: Description of Artificial Sequence: Synthetic polynucleotide
  source 1..30: mol_type = other DNA
atccttatcgttatgggtattgtttgtaat

SEQ ID NO: 103 - DNA, 30 bp, synthetic construct
  misc_feature 1..30: note = Synthetic: Description of Artificial Sequence: Synthetic polynucleotide
  source 1..30: mol_type = other DNA
taaaagaattgtgagcgggaatacaacaac

SEQ ID NO: 104 - DNA, 30 bp, synthetic construct
  misc_feature 1..30: note = Synthetic: Description of Artificial Sequence: Synthetic polynucleotide
  source 1..30: mol_type = other DNA
tacaaaataattcccctgcaaacattatca

SEQ ID NO: 105 - DNA, 30 bp, synthetic construct
  misc_feature 1..30: note = Synthetic: Description of Artificial Sequence: Synthetic polynucleotide
  source 1..30: mol_type = other DNA
tacaaaataattcccctgcaaacattatcg

SEQ ID NO: 106 - DNA, 30 bp, synthetic construct
  misc_feature 1..30: note = Synthetic: Description of Artificial Sequence: Synthetic polynucleotide
  source 1..30: mol_type = other DNA
agggaatacaagctacttgttctttttgca

SEQ ID NO: 107 - DNA, 23 bp, synthetic construct
  misc_feature 1..23: note = Synthetic: Description of Artificial Sequence: Synthetic polynucleotide
  source 1..23: mol_type = other DNA
taatacgactcactatagggaga

SEQ ID NO: 108 - DNA, 28 bp, synthetic construct
  misc_feature 1..28: note = Synthetic: Description of Artificial Sequence: Synthetic polynucleotide
  source 1..28: mol_type = other DNA
gaatttaatacgactcactatagggaga

SEQ ID NO: 109 - DNA, 19 bp, synthetic construct
  misc_feature 1..19: note = Synthetic: Description of Artificial Sequence: Synthetic polynucleotide
  source 1..19: mol_type = other DNA
taatacgactcactatagg

SEQ ID NO: 110 - DNA, 30 bp, synthetic construct
  misc_feature 1..30: note = Synthetic: Description of Artificial Sequence: Synthetic polynucleotide
  source 1..30: mol_type = other DNA
gagtcgtattaatacgactcactatagggg

SEQ ID NO: 111 - DNA, 30 bp, synthetic construct
  misc_feature 1..30: note = Synthetic: Description of Artificial Sequence: Synthetic polynucleotide
  source 1..30: mol_type = other DNA
agtgagtcgtactacgactcactatagggg

SEQ ID NO: 112 - DNA, 30 bp, synthetic construct
  misc_feature 1..30: note = Synthetic: Description of Artificial Sequence: Synthetic polynucleotide
  source 1..30: mol_type = other DNA
gagtcgtattaatacgactctctatagggg

SEQ ID NO: 113 - DNA, 18 bp, synthetic construct
  misc_feature 1..18: note = Synthetic: Description of Artificial Sequence: Synthetic polynucleotide
  source 1..18: mol_type = other DNA
taatacgactcactatag

SEQ ID NO: 114 - DNA, 23 bp, synthetic construct
  misc_feature 1..23: note = Synthetic: Description of Artificial Sequence: Synthetic polynucleotide
  source 1..23: mol_type = other DNA
ttatacgactcactatagggaga

SEQ ID NO: 115 - DNA, 23 bp, synthetic construct
  misc_feature 1..23: note = Synthetic: Description of Artificial Sequence: Synthetic polynucleotide
  source 1..23: mol_type = other DNA
gaatacgactcactatagggaga

SEQ ID NO: 116 - DNA, 23 bp, synthetic construct
  misc_feature 1..23: note = Synthetic: Description of Artificial Sequence: Synthetic polynucleotide
  source 1..23: mol_type = other DNA
taatacgtctcactatagggaga

SEQ ID NO: 117 - DNA, 23 bp, synthetic construct
  misc_feature 1..23: note = Synthetic: Description of Artificial Sequence: Synthetic polynucleotide
  source 1..23: mol_type = other DNA
tcatacgactcactatagggaga

SEQ ID NO: 118 - DNA, 30 bp, synthetic construct
  misc_feature 1..30: note = Synthetic: Description of Artificial Sequence: Synthetic polynucleotide
  source 1..30: mol_type = other DNA
taatacgactcactatagggagaccacaac

SEQ ID NO: 119 - DNA, 30 bp, synthetic construct
  misc_feature 1..30: note = Synthetic: Description of Artificial Sequence: Synthetic polynucleotide
  source 1..30: mol_type = other DNA
taattgaactcactaaagggagaccacagc

SEQ ID NO: 120 - DNA, 30 bp, synthetic construct
  misc_feature 1..30: note = Synthetic: Description of Artificial Sequence: Synthetic polynucleotide
  source 1..30: mol_type = other DNA
cgaagtaatacgactcactattagggaaga

SEQ ID NO: 121 - DNA, 19 bp, synthetic construct
  misc_feature 1..19: note = Synthetic: Description of Artificial Sequence: Synthetic polynucleotide
  source 1..19: mol_type = other DNA
atttaggtgacactataga

SEQ ID NO: 122 - DNA, 30 bp, synthetic construct
  misc_feature 1..30: note = Synthetic: Description of Artificial Sequence: Synthetic polynucleotide
  source 1..30: mol_type = other DNA
acaaacacaaatacacacactaaattaata

SEQ ID NO: 123 - DNA, 30 bp, synthetic construct
  misc_feature 1..30: note = Synthetic: Description of Artificial Sequence: Synthetic polynucleotide
  source 1..30: mol_type = other DNA
ccaagcatacaatcaactatctcatataca

SEQ ID NO: 124 - DNA, 30 bp, synthetic construct
  misc_feature 1..30: note = Synthetic: Description of Artificial Sequence: Synthetic polynucleotide
  source 1..30: mol_type = other DNA
gatacaggatacagcggaaacaacttttaa

SEQ ID NO: 125 - DNA, 30 bp, synthetic construct
  misc_feature 1..30: note = Synthetic: Description of Artificial Sequence: Synthetic polynucleotide
  source 1..30: mol_type = other DNA
tttcaagctataccaagcatacaatcaact

SEQ ID NO: 126 - DNA, 30 bp, synthetic construct
  misc_feature 1..30: note = Synthetic: Description of Artificial Sequence: Synthetic polynucleotide
  source 1..30: mol_type = other DNA
cctttgcagcataaattactatacttctat

SEQ ID NO: 127 - DNA, 30 bp, synthetic construct
  misc_feature 1..30: note = Synthetic: Description of Artificial Sequence: Synthetic polynucleotide
  source 1..30: mol_type = other DNA
ttatctactttttacaacaaatataaaaca

SEQ ID NO: 128 - DNA, 30 bp, synthetic construct
  misc_feature 1..30: note = Synthetic: Description of Artificial Sequence: Synthetic polynucleotide
  source 1..30: mol_type = other DNA
gtttcgaataaacacacataaacaaacaaa

SEQ ID NO: 129 - DNA, 30 bp, synthetic construct
  misc_feature 1..30: note = Synthetic: Description of Artificial Sequence: Synthetic polynucleotide
  source 1..30: mol_type = other DNA
accatcaaaggaagctttaatcttctcata

SEQ ID NO: 130 - DNA, 30 bp, synthetic construct
  misc_feature 1..30: note = Synthetic: Description of Artificial Sequence: Synthetic polynucleotide
  source 1..30: mol_type = other DNA
agaacccactgcttactggcttatcgaaat

SEQ ID NO: 131 - DNA, 30 bp, synthetic construct
  misc_feature 1..30: note = Synthetic: Description of Artificial Sequence: Synthetic polynucleotide
  source 1..30: mol_type = other DNA
ggccgtttttggcttttttgttagacgaag

SEQ ID NO: 132 - DNA, 52 bp, synthetic construct
  misc_feature 1..52: note = Synthetic: Description of Artificial Sequence: Synthetic polynucleotide
  source 1..52: mol_type = other DNA
aaaaagagtattgacttcgcatctttttgtacctataatagattcattgcta

SEQ ID NO: 133 - DNA, 59 bp, synthetic construct
  misc_feature 1..59: note = Synthetic: Description of Artificial Sequence: Synthetic polynucleotide
  source 1..59: mol_type = other DNA
ggaaaatttttttaaaaaaaaaactttacagctagctcagtcctaggtattatgctagc

SEQ ID NO: 134 - DNA, 59 bp, synthetic construct
  misc_feature 1..59: note = Synthetic: Description of Artificial Sequence: Synthetic polynucleotide
  source 1..59: mol_type = other DNA
ggaaaatttttttaaaaaaaaaactttacggctagctcagccctaggtattatgctagc

SEQ ID NO: 135 - DNA, 64 bp, synthetic construct
  misc_feature 1..64: note = Synthetic: Description of Artificial Sequence: Synthetic polynucleotide
  source 1..64: mol_type = other DNA
ggaaaatttttttaaaaaaaaaacttgacagctagctcagtccttggtataatgctagcacgaa

SEQ ID NO: 136 - protein, 21 aa, synthetic construct
  REGION 1..21: note = Synthetic: PhoA secretion tag
MKQSTIALALLPLLFTPVTKA

SEQ ID NO: 137 - protein, 20 aa, synthetic construct
  REGION 1..20: note = Synthetic: PhoA secretion tag
KQSTIALALLPLLFTPVTKA

SEQ ID NO: 138 - protein, 22 aa, synthetic construct
  REGION 1..22: note = Synthetic: OmpF secretion tag
MMKRNILAVIVPALLVAGTANA

SEQ ID NO: 139 - protein, 15 aa, synthetic construct
  REGION 1..15: note = Synthetic: cvaC secretion tag
MRTLTLNELDSVSGG

SEQ ID NO: 140 - protein, 43 aa, synthetic construct
  REGION 1..43: note = Synthetic: TorA secretion tag
MNNNDLFQASRRRFLAQLGGLTVAGMLGTSLLTPRRATAAQAA

SEQ ID NO: 141 - protein, 33 aa, synthetic construct
  REGION 1..33: note = Synthetic: fdnG secretion tag
MDVSRRQFFKICAGGMAGTTVAALGFAPKQALA

SEQ ID NO: 142 - protein, 45 aa, synthetic construct
  REGION 1..45: note = Synthetic: dmsA secretion tag
MKTKIPDAVLAAEVSRRGLVKTTAIGGLAMASSALTLPFSRIAHA

SEQ ID NO: 143 - protein, 21 aa, synthetic construct
  REGION 1..21: note = Synthetic: PelB secretion tag
KYLLPTAAAGLLLLAAQPAMA

SEQ ID NO: 144 - protein, 52 aa, synthetic construct
  REGION 1..52: note = Synthetic: HlyA secretion signal
LNPLINEISKIISAAGNFDVKEERAAASLLQLSGNASDFSYGRNSITLTASA

SEQ ID NO: 145 - DNA, 159 bp, synthetic construct
  misc_feature 1..159: note = Synthetic: HlyA secretion signal
  source 1..159: mol_type = other DNA
cttaatccattaattaatgaaatcagcaaaatcatttcagctgcaggtaattttgatgttaaagaggaaagagctgcagcttctttattgcagttgtccggtaatgccagtgatttttcatatggacggaactcaataactttgacagcatcagcataa

SEQ ID NO: 146 - protein, 52 aa, synthetic construct
  REGION 1..52: note = Synthetic: ECOLIN_05715 Secretion signal
MKRHLNTSYRLVWNHITGAFVVASELARARGKRAGVAVALSLAAATSLPALA

SEQ ID NO: 147 - protein, 40 aa, synthetic construct
  REGION 1..40: note = Synthetic: ECOLIN_16495 Secretion signal
MFWRDMTLSVWRKKTTGLKTKKRLLALVLAAALCSSPVWA

SEQ ID NO: 148 - protein, 33 aa, synthetic construct
  REGION 1..33: note = Synthetic: ECOLIN_19410 Secretion signal
MGYKMNISSLRKAFIFMGAVAALSLVNAQSALA

SEQ ID NO: 149 - protein, 53 aa, synthetic construct
  REGION 1..53: note = Synthetic: ECOLIN_19880 Secretion signal
MNKIFKVIWNPATGSYTVASETAKSRGKKSGRSKLLISALVAGGLLSSFGASA

SEQ ID NO: 150 - DNA, 159 bp, synthetic construct
  misc_feature 1..159: note = Synthetic: adhesion Secretion signal
  source 1..159: mol_type = other DNA
atgaacaagattttcaaggttatctggaaccccgcgacaggcagctacactgtggcctccgaaaccgctaagtcgcgcggcaagaagtcaggacgttcaaagctgcttatttctgctttagtagccggaggattactttctagctttggagcctcggca

SEQ ID NO: 151 - protein, 19 aa, synthetic construct
  REGION 1..19: note = Synthetic: DsbA secretion tag
MKKIWLALAGLVLAFSASA

SEQ ID NO: 152 - DNA, 57 bp, synthetic construct
  misc_feature 1..57: note = Synthetic: DsbA secretion tag
  source 1..57: mol_type = other DNA
atgaaaaaaatctggctggcattagcaggcttagtcctggcctttagtgcctcggca

SEQ ID NO: 153 - protein, 22 aa, synthetic construct
  REGION 1..22: note = Synthetic: GltI secretion tag
MQLRKPATAILALALSAGLAQA

SEQ ID NO: 154 - DNA, 66 bp, synthetic construct
  misc_feature 1..66: note = Synthetic: GltI secretion tag
  source 1..66: mol_type = other DNA
atgcagttgcgcaagcctgccacggcaattttagcattagctctttctgcgggcttggcccaggca

SEQ ID NO: 155 - DNA, 120 bp, synthetic construct
  misc_feature 1..120: note = Synthetic: GspD secretion tag
  source 1..120: mol_type = other DNA
atgttctggcgcgacatgacactgagcgtttggcgtaaaaaaacaacgggactgaaaaccaaaaagcgtttgttagcccttgtcctggcggcagccctgtgctcgtccccggtatgggca

SEQ ID NO: 156 - DNA, 99 bp, synthetic construct
  misc_feature 1..99: note = Synthetic: HdeB secretion tag
  source 1..99: mol_type = other DNA
atgggatacaagatgaacatttcatcacttcgcaaagcttttatcttcatgggtgccgttgctgctctgtcactggtcaatgctcaatcggccctggca

SEQ ID NO: 157 - protein, 26 aa, synthetic construct
  REGION 1..26: note = Synthetic: MalE secretion tag
MKIKTGARILALSALTTMMFSASALA

SEQ ID NO: 158 - DNA, 78 bp, synthetic construct
  misc_feature 1..78: note = Synthetic: MalE secretion tag
  source 1..78: mol_type = other DNA
atgaagattaaaacaggcgctcgtattttggccctgtctgcgcttacgactatgatgtttagtgcctcggccctggca

SEQ ID NO: 159 - protein, 26 aa, synthetic construct
  REGION 1..26: note = Synthetic: OppA secretion tag
MTNITKRSLVAAGVLAALMAGNVALA

SEQ ID NO: 160 - DNA, 78 bp, synthetic construct
  misc_feature 1..78: note = Synthetic: OppA secretion tag
  source 1..78: mol_type = other DNA
atgacgaatatcactaaacgtagccttgtggccgcgggggtcttggctgcattgatggctggcaatgttgctctggca

SEQ ID NO: 161 - protein, 22 aa, synthetic construct
  REGION 1..22: note = Synthetic: PelB secretion tag
MKYLLPTAAAGLLLLAAQPAMA

SEQ ID NO: 162 - DNA, 66 bp, synthetic construct
  misc_feature 1..66: note = Synthetic: PelB secretion tag
  source 1..66: mol_type = other DNA
atgaaatatctgttgcccacggctgccgcgggtctgctgctgctggcagcgcaaccggctatggca

SEQ ID NO: 163 - DNA, 63 bp, synthetic construct
  misc_feature 1..63: note = Synthetic: PhoA secretion tag
  source 1..63: mol_type = other DNA
atgaagcaaagtacgatcgcccttgcgctgttgccccttcttttcacgcccgtcaccaaggca

SEQ ID NO: 164 - protein, 24 aa, synthetic construct
  REGION 1..24: note = Synthetic: PpiA secretion tag
MFKSTLAAMAAVFALSALSPAAMA

SEQ ID NO: 165 - DNA, 72 bp, synthetic construct
  misc_feature 1..72: note = Synthetic: PpiA secretion tag
  source 1..72: mol_type = other DNA
atgtttaagtctacacttgcagccatggccgcagtcttcgcactgtcagccttgagtcctgctgcaatggca

SEQ ID NO: 166 - protein, 21 aa, synthetic construct
  REGION 1..21: note = Synthetic: tolb Secretion Tag
MKQALRVAFGFLILWASVLHA

SEQ ID NO: 167 - protein, 18 aa, synthetic construct
  REGION 1..18: note = Synthetic: tort Secretion Tag
MRVLLFLLLSLFMLPAFS

SEQ ID NO: 168 - protein, 21 aa, synthetic construct
  REGION 1..21: note = Synthetic: ompA Secretion Tag
MKKTAIAIAVALAGFATVAQA

SEQ ID NO: 169 - DNA, 57 bp, synthetic construct
  misc_feature 1..57: note = Synthetic: dsba Secretion Tag
  source 1..57: mol_type = other DNA
atgaagaaaatttggctggcacttgccggtttggtattagcgttttccgcctcggca

SEQ ID NO: 170 - DNA, 63 bp, synthetic construct
  misc_feature 1..63: note = Synthetic: tolb secretion tag
  source 1..63: mol_type = other DNA
atgaagcaggcgcttcgcgttgcgtttgggttccttatcctgtgggcatccgtacttcacgcc

SEQ ID NO: 171 - DNA, 54 bp, synthetic construct
  misc_feature 1..54: note = Synthetic: tort secretion tag
  source 1..54: mol_type = other DNA
atgcgcgtactgttattccttctgttgtctctttttatgttgcccgctttcagt

SEQ ID NO: 172 - DNA, 63 bp, synthetic construct
  misc_feature 1..63: note = Synthetic: ompA secretion tag
  source 1..63: mol_type = other DNA
atgaagaagactgccatcgctattgccgtcgcccttgcgggtttcgcaaccgtggcgcaagca

SEQ ID NO: 173 - DNA, 66 bp, synthetic construct
  misc_feature 1..66: note = Synthetic: pelB secretion tag
  source 1..66: mol_type = other DNA
atgaaatatctgcttccaacggctgctgctggcctgttgcttcttgccgcccagcctgcgatggct

SEQ ID NO: 174 - DNA, 99 bp, synthetic construct
  misc_feature 1..99: note = Synthetic: 19410 secretion tag
  source 1..99: mol_type = other DNA
atggggtacaaaatgaacattagctcgcttcgcaaagcattcatttttatgggggctgttgcagctttaagccttgtcaatgcccagtcagcgcttgcc

SEQ ID NO: 175 - DNA, 57 bp, synthetic construct
  misc_feature 1..57: note = Synthetic: dsba secretion tag
  source 1..57: mol_type = other DNA
atgaaaaagatttggctggctcttgccggtttagtcctggcattcagcgcaagcgcg

SEQ ID NO: 176 - DNA, 63 bp, synthetic construct
  misc_feature 1..63: note = Synthetic: phoA secretion tag
  source 1..63: mol_type = other DNA
atgaaacagagcacaatcgctttagccttgctgcctcttcttttcacgcctgtcacgaaggca

SEQ ID NO: 177 - DNA, 63 bp, synthetic construct
  misc_feature 1..63: note = Synthetic: tolB secretion tag
  source 1..63: mol_type = other DNA
atgaagcaggctctgcgcgtggcatttgggttcctgatcttatgggcgtccgtcttacacgca

SEQ ID NO: 178 - DNA, 78 bp, synthetic construct
  misc_feature 1..78: note = Synthetic: malE secretion tag
  source 1..78: mol_type = other DNA
atgaaaatcaagacgggggcacgcattcttgccttgagcgccttgacaacgatgatgttcagtgcaagtgcattagcg

SEQ ID NO: 179 - DNA, 69 bp, synthetic construct
  misc_feature 1..69: note = Synthetic: mglB secretion tag
  source 1..69: mol_type = other DNA
atgaataagaaagtcttgacattgtcggcggtcatggcctcgatgttgttcggggcagcagcccacgca

SEQ ID NO: 180 - DNA, 66 bp, synthetic construct
  misc_feature 1..66: note = Synthetic: ompF secretion tag
  source 1..66: mol_type = other DNA
atgatgaagcgtaacatcttagccgttattgtccccgcattgcttgtggccgggacggctaacgca

SEQ ID NO: 181 - DNA, 63 bp, synthetic construct; identical to SEQ ID NO: 172
  misc_feature 1..63: note = Synthetic: ompA secretion tag
  source 1..63: mol_type = other DNA

SEQ ID NO: 182 - DNA, 54 bp, synthetic construct
  misc_feature 1..54: note = Synthetic: tort secretion tag
  source 1..54: mol_type = other DNA
atgcgcgtgttattgttcttgctgctgagcttgtttatgttaccagctttcagt

SEQ ID NO: 183 - DNA, 75 bp, synthetic construct
  misc_feature 1..75: note = Synthetic: lamB secretion tag
  source 1..75: mol_type = other DNA
atgatgattaccttacgcaagttgccccttgcggtagccgttgctgctggtgtgatgtccgcgcaagcaatggct

SEQ ID NO: 184 - DNA, 66 bp, synthetic construct
  misc_feature 1..66: note = Synthetic: pelB secretion tag
  source 1..66: mol_type = other DNA
atgaaatatcttcttccaacggctgctgctggtttattgcttcttgccgcccagcctgcgatggct

SEQ ID NO: 185 - protein, 23 aa, synthetic construct
  REGION 1..23: note = Synthetic: secretion tag
MNKKVLTLSAVMASMLFGAAAHA

SEQ ID NO: 186 - protein, 25 aa, synthetic construct
  REGION 1..25: note = Synthetic: secretion tag
MMITLRKLPLAVAVAAGVMSAQAMA

SEQ ID NO: 187 - DNA, 65 bp, synthetic construct
  misc_feature 1..65: note = Synthetic: secretion tag
  source 1..65: mol_type = other DNA
tgcagttgcgcaagcctgccacggcaattttagcattagctctttctgcgggcttggcccaggca

SEQ ID NO: 188 - DNA, 121 bp, synthetic construct
  misc_feature 1..121: note = Synthetic: secretion tag
  source 1..121: mol_type = other DNA
aatgttctggcgcgacatgacactgagcgtttggcgtaaaaaaacaacgggactgaaaaccaaaaagcgtttgttagcccttgtcctggcggcagccctgtgctcgtccccggtatgggca

SEQ ID NO: 189 - DNA, 57 bp, synthetic construct; identical to SEQ ID NO: 169
  misc_feature 1..57: note = Synthetic: dsba secretion tag
  source 1..57: mol_type = other DNA

SEQ ID NO: 190 - protein, 607 aa, synthetic construct
  REGION 1..607: note = Synthetic: Invasin display tag
MVFQPISEFLLIRNAGMSMYFNKIISFNIISRIVICIFLICGMFMAGASEKYDANAPQQVQPYSVSSSAFENLHPNNEMESSINPFSASDTERNAAIIDRANKEQETEAVNKMISTGARLAASGRASDVAHSMVGDAVNQEIKQWLNRFGTAQVNLNFDKNFSLKESSLDWLAPWYDSASFLFFSQLGIRNKDSRNTLNLGVGIRTLENGWLYGLNTFYDNDLTGHNHRIGLGAEAWTDYLQLAANGYFRLNGWHSSRDFSDYKERPATGGDLRANAYLPALPQLGGKLMYEQYTGERVALFGKDNLQRNPYAVTAGINYTPVPLLTVGVDQRMGKSSKHETQWNLQMNYRLGESFQSQLSPSAVAGTRLLAESRYNLVDRNNNIVLEYQKQQVVKLTLSPATISGLPGQVYQVNAQVQGASAVREIVWSDAELIAAGGTLTPLSTTQFNLVLPPYKRTAQVSRVTDDLTANFYSLSALAVDHQGNRSNSFTLSVTVQQPQLTLTAAVIGDGAPANGKTAITVEFTVADFEGKPLAGQEVVITTNNGALPNKITEKTDANGVARIALTNTTDGVTVVTAEVEGQRQSVDTHFVKGTIAADKSTLAAV

SEQ ID NO: 191 - protein, 148 aa, synthetic construct
  REGION 1..148: note = Synthetic: LppOmpA display tag
KATKLVLGAVILGSTLLAGCSSNAKIDQGINPYVGFEMGYDWLGRMPYKGSVENGAYKAQGVQLTAKLGYPITDDLDIYTRLGGMVWRADTKSNVYGKNHDTGVSPVFAGGVEYAITPEIATRLEYQWTNNIGDAHTIGTRPDNGIPG

SEQ ID NO: 192 - protein, 658 aa, synthetic construct
  REGION 1..658: note = Synthetic: IntiminN display tag
ITHGCYTRTRHKHKLKKTLIMLSAGLGLFFYVNQNSFANGENYFKLGSDSKLLTHDSYQNRLFYTLKTGETVADLSKSQDINLSTIWSLNKHLYSSESEMMKAAPGQQIILPLKKLPFEYSALPLLGSAPLVAAGGVAGHTNKLTKMSPDVTKSNMTDDKALNYAAQQAASLGSQLQSRSLNGDYAKDTALGIAGNQASSQLQAWLQHYGTAEVNLQSGNNFDGSSLDFLLPFYDSEKMLAFGQVGARYIDSRFTANLGAGQRFFLPANMLGYNVFIDQDFSGDNTRLGIGGEYWRDYFKSSVNGYFRMSGWHESYNKKDYDERPANGFDIRFNGYLPSYPALGAKLIYEQYYGDNVALFNSDKLQSNPGAATVGVNYTPIPLVTMGIDYRHGTGNENDLLYSMQFRYQFDKSWSQQIEPQYVNELRTLSGSRYDLVQRNNNIILEYKKQDILSLNIPHDINGTEHSTQKIQLIVKSKYGLDRIVWDDSALRSQGGQIQHSGSQSAQDYQAILPAYVQGGSNIYKVTARAYYRNGNSSNNVQLTITVLSNGQVVDQVGVTDFTADKTSAKADNADTITYTATVKKNGVAQANVPVSFNIVSGTATLGANSAKTDANGKATVTLKSSTPGQVVVSAKTAEMTSALNASAVIFFDQTKAS

SEQ ID NO: 193 - DNA, 456 bp, synthetic construct
  misc_feature 1..456: note = Synthetic: pelB anchor
  source 1..456: mol_type = other DNA
tgcagtagcaataaaaatgcttcgaatgacggatcggaaggaatgttaggtgctggtactggcatggacgccaacggtggcaacggaaatatgtctagcgaagaacaagctcgtttacaaatgcagcaattacagcaaaacaacatagtctacttcgacttagacaagtacgacatccgtagcgattttgcacaaatgttggatgcccatgccaacttccttcgttctaatccaagctacaaagtcactgttgagggccacgcggatgagagaggcactcctgaatataacatatccctgggggagcgcagagcaaacgccgtaaaaatgtacctgcaaggcaagggcgtctcggccgatcaaatctcgatcgtgagctatggcaaagagaaaccggctgtattgggacatgacgaagctgcttacagcaagaatcggcgggccgtcttggtctac

SEQ ID NO: 194 - DNA, 63 bp, synthetic construct
  misc_feature 1..63: note = Synthetic: BAN anchor
  source 1..63: mol_type = other DNA
gcttttgaccccaatctggttgggcctacgttacctccaattccgcctttcactctgcctacg

SEQ ID NO: 195 - DNA, 442 bp, synthetic construct
  misc_feature 1..442: note = Synthetic: LppOmpA anchor
  source 1..442: mol_type = other DNA
atgactagttaaagcaacaaaacttgtgttaggcgcggttatacttggctccaccctgcttgcaggttgctcgtctaacgcgaagatcgaccagggtatcaatccttacgtcgggtttgaaatgggatacgattggttgggacgtatgccttataagggaagtgttgaaaacggcgcttataaggcgcagggagtacagttaacggccaagcttgggtaccccataacagacgatttagatatttatacccgtttaggaggaatggtttggagagccgacacgaagtctaatgtatatggtaagaaccacgacacgggagtatcccccgtctttgcagggggagtggaatatgctatcacaccagagatcgctacccgtttggaatatcaatggacgaataatataggcgacgcccatacgataggaacgcggcccgacaac

SEQ ID NO: 196 - DNA, 915 bp, synthetic construct
  misc_feature 1..915: note = Synthetic: NGIgAsig anchor
  source 1..915: mol_type = other DNA
ccgcgtgctgcacaaccgcgtacacaagctgcagcccaggccgacgcagtatccacgaacacaaattcagcgttgagtgatgctatggcttcaactcaatcaattcttttggacaccggcgcatatttaactcgccatatcgcccaaaagtctcgtgcggatgcagaaaaaaatagcgtgtggatgtcgaatactgggtatgggcgcgactacgcctctgcgcagtaccgccgtttcagcagcaaacgcacccagactcaaatcgggatcgatcgtagtcttagtgagaatatgcagattggcggagttctgacttattccgactctcaacatacgttcgaccaggcagggggcaagaatacgtttgtacaggccaatctttacggaaagtactacttgaatgatgcctggtatgtcgctggggacattggcgcagggagcttgcgttcccgccttcaaacgcaacaaaaggcgaatttcaatcgcaccagcattcagactggccttacacttgggaatacacttaagattaatcaatttgagattgtgcccagtgctgggattcgttatagtcgtttatccagcgcagactataagttaggcgacgactctgtgaaagttagctcaatggccgtgaagacgttgacggctggtttggatttcgcctatcgctttaaagttggaaatctgaccgtgaagccgctgctttcggccgcttattttgcaaactacggcaagggcggcgttaatgtcggtggtaagagtttcgcctataaagctgacaatcagcagcaatattcggccggcgtcgcgttgttgtatcgcaacgtgactttaaatgtaaacggctccatcacaaagggcaagcagttggagaagcaaaagtcaggccaaatcaagattcaaatccgtttc

SEQ ID NO: 197 - DNA, 603 bp, synthetic construct
  misc_feature 1..603: note = Synthetic: OsmY anchor
  source 1..603: mol_type = other DNA
atgacaatgactcgtttgaaaatttccaaaaccctgctggccgtaatgcttacgtccgcagtcgccactggttccgcatacgcagagaataatgcgcagacaactaacgagtcagcagggcaaaaagtagactccagtatgaataaggtgggcaattttatggacgactcagctatcaccgctaaggtgaaagcggcgctggtggaccacgacaacatcaagtccacggacatctcagttaaaacggaccaaaaggtagtaaccttaagcgggttcgtagaaagccaggcgcaagcagaggaagcggtaaaagtcgctaaaggcgtagaaggggtcacttcggtgtcagacaagttgcatgtgcgtgacgcaaaggaaggatcagtaaagggttatgccggagatacggcaacgacctctgagatcaaagcgaaattactggcagacgacattgttccctcgcgtcatgttaaagtcgaaacgactgatggcgtagtccagctttcgggtacagttgactcccaagcacaaagtgatcgcgcggaatctattgcaaaggcggtggacggagtgaaatccgtcaaaaacgatcttaaaacgaaa

SEQ ID NO: 198 - DNA, 1820 bp, synthetic construct
  misc_feature 1..1820: note = Synthetic: invasinN anchor
  source 1..1820: mol_type = other DNA
atggttttccaacccatcagcgaatttttgctgattcgtaacgctgggatgtccatgtattttaacaagatcatttcttttaacatcatttcacgtatcgttatttgcatttttcttatctgtggtatgttcatggccggtgcatctgaaaagtatgatgcaaacgcaccccaacaggtgcagccatactcggtttcatcatcagcgttcgagaatctgcaccccaataacgagatggagtcgagtatcaacccttttagtgcttcggacaccgagcgtaatgcagctatcatcgatcgtgctaacaaggaacaagaaacggaagcagtcaacaaaatgatctccactggcgctcgtttagctgccagcggtcgcgcgtccgatgtggcgcacagtatggtaggggatgcggtcaaccaggagattaaacaatggctgaatcgcttcggcactgctcaagtgaatttaaattttgacaagaacttctcgttaaaggagtcttcgcttgactggttggccccatggtacgattcggcgtcattccttttcttttctcagttgggcatccgtaacaaggacagtcgtaatacacttaaccttggtgttggcattcgcacattagaaaatggttggttgtatggcctgaacaccttttacgacaatgacttaacgggacacaatcaccgtatcgggctgggcgccgaggcgtggactgactacttgcagttagccgcgaatgggtacttccgtcttaatggttggcactcttcccgtgacttcagcgactacaaagaacgccctgctaccgggggagatttgcgtgcgaatgcgtacctgcccgctcttccgcaacttggcgggaagttaatgtatgagcagtatactggggaacgcgtggctctgttcggaaaggacaacctgcagcgcaacccatacgctgtcactgcgggtatcaactatacgccagttccgttgctgacggtcggcgtggatcaacgtatggggaagtcgagtaaacatgaaacgcaatggaatttacaaatgaactatcgcttaggggagagtttccaaagtcagcttagcccttcggcggtcgcagggactcgtttgcttgctgagtcccgctacaacctggttgatcgcaataacaatatcgtactggaataccagaaacaacaagtggttaagctgacgttgagccctgcgaccatcagtggattgcccggacaagtttaccaggtaaatgcccaggtccagggggcctctgcggttcgcgaaattgtctggtcagacgcagaattaatcgctgcaggaggcaccttaacgccactttccactacacaattcaatttagtccttcccccatacaaacgtaccgcccaggtatcgcgcgtaactgatgacttaactgctaatttttattcactgtcggcgttagcagttgaccatcaaggcaaccgtagtaattccttcacattatctgtaacggtgcagcagccgcaactgacgcttaccgcagcggtcattggtgatggggccccagctaatgggaaaaccgcaatcactgtcgagttcacagttgcagattttgaaggcaagccgctggcgggtcaggaggttgtgattacgactaataacggtgctcttcctaataagattactgaaaagactgacgctaacggcgttgcccgcattgcccttacgaacacaaccgatggggtcacggtagttaccgcagaggtcgaggggcaacgccaatccgttgacacgcacttcgttaagggtactatcgcggccgataaaagcacgctggccgcggt

SEQ ID NO: 199 - DNA, 696 bp, synthetic construct
  misc_feature 1..696: note = Synthetic: yiaT anchor
  source 1..696: mol_type = other DNA
atgttaatcaatagaaacattgtcgccctttttgccctgccattcatggcgagtgcgaccgcttccgagttaagcataggggccggcgctgcgtataatgagagtccttaccgtggctataatgaaaacacaaaggcaatcccccttatctcgtatgaaggcgacaccttctatgtacgtcagactacattaggtttcatcttaagccagtccgagaaaaacgagttatcgctgacagcttcatggatgccattggaattcgacccgacagataatgatgattatgcgatgcagcagctggacaaacgggattcgacagctatggccggcgtggcttggtatcaccacgagcggtggggaactgtcaaagcttccgccgccgctgacgtgttagacaacagcaatggctgggtgggggaattaagcgtgttccataagatgcagataggtagattatcccttacaccggctttgggagtcctgtattatgatgagaacttttccgactattattatgggatttctgagtctgaaagtcgtcggagcggtcttgcgtcgtactcggcgcaagacgcgtgggtgccttatgtatctttgacagcgaagtatccaataggcgagcacgtggtattgatggcaagtgctggttattccgagttgcccgaagaaattactgacagtcctatgatagaccgt

SEQ ID NO: 200 - DNA, 1977 bp, synthetic construct
  misc_feature 1..1977: note = Synthetic: intiminN anchor
  source 1..1977: mol_type = other DNA
atgattacgcatggctgttatacccgtacgcgtcataaacacaagttgaagaaaactctgatcatgttatccgctggacttggactttttttttacgtgaatcagaactctttcgctaatggggaaaattattttaaactgggatcagacagcaaattacttacgcatgactcataccagaatcgtctgttttatacgctgaaaactggtgaaaccgttgcagatttaagtaaaagtcaggacattaacctgtcaactatttggtcacttaataaacacttatattcgagcgaatcggaaatgatgaaagctgcaccggggcaacaaatcatcttgcccctgaagaaattgccctttgaatactccgctttgcccttgctgggctcggctcctctggtagccgccggaggcgttgccggtcacactaataagctgacaaaaatgtcacccgacgtgacgaagagcaacatgacggatgataaggctttaaattacgcagctcagcaagcggcctcgttgggaagtcagttacagagtcgttcgttaaatggtgattatgctaaggataccgcattgggtattgccggcaaccaagcgtcgagccaacttcaggcatggttgcaacattacggcactgctgaagtaaatctgcaatcaggtaataattttgacggtagttccctggatttccttttacctttttacgattcagaaaagatgttggctttcggacaggtgggggcgcgttacatcgattcacgttttaccgctaacttgggggccggtcaacgcttcttcttacctgccaatatgttgggctataatgtatttatcgaccaggacttcagtggtgacaatacacgtctgggaattggtggagagtattggcgcgattactttaagtcatctgtaaatggctattttcgcatgagcggttggcatgaaagttacaacaagaaagactacgatgagcgccccgcgaacgggtttgacatccgttttaatggttatttgccatcttatcccgccttgggagctaaattaatctacgagcaatactatggagataacgtagctttgtttaatagcgacaagttacagtctaatccaggagcggctacagtgggagttaattataccccaatcccactggtcacaatgggaatcgattatcgccacgggactggtaatgaaaacgatttattatactccatgcagtttcgttatcagttcgataagagttggtcgcagcagattgagcctcaatatgttaacgaattacgtaccttgtccggcagtcgctacgatctggtacaacgcaataacaatatcatccttgagtataagaaacaggacattctgtctttgaacattccacatgatattaatggtaccgagcactcaacacaaaaaattcagctgattgtgaaatcaaagtatggactggaccgtatcgtgtgggatgatagcgctctgcgcagtcagggtggacagatccagcactcgggtagccagtctgcccaagactaccaggctatcctgccagcgtatgtccaagggggaagtaacatctacaaagttacagctcgcgcctatgaccgcaacggtaattctagtaataatgtgcagttgacaattacggtgctgtccaatgggcaggtcgtcgatcaggtaggtgtgacggattttacagccgataaaacctctgcgaaggcagataacgcggataccatcacatacactgccactgtaaaaaaaaacggtgtcgcgcaggcaaacgttcctgttagcttcaacatcgtgtcgggtacagccacccttggggccaactcggcaaagactgacgcgaatggcaaggctacagtcacgttgaaatcctcgacaccaggacaggtcgttgtgtctgccaagacagcagagatgacctccgcccttaatgcatctgctgttatcttcttcgatcaaacgaaggcatct

SEQ ID NO: 201 - protein, 152 aa, synthetic construct
  REGION 1..152: note = Synthetic: pelB anchor
CSSNKNASNDGSEGMLGAGTGMDANGGNGNMSSEEQARLQMQQLQQNNIVYFDLDKYDIRSDFAQMLDAHANFLRSNPSYKVTVEGHADERGTPEYNISLGERRANAVKMYLQGKGVSADQISIVSYGKEKPAVLGHDEAAYSKNRRAVLVY

SEQ ID NO: 202 - protein, 24 aa, synthetic construct
  REGION 1..24: note = Synthetic: BAN anchor
MTSAFDPNLVGPTLPPIPPFTLPT

SEQ ID NO: 203 - protein, 147 aa, synthetic construct
  REGION 1..147: note = Synthetic: LppOmpA anchor
MTSKATKLVLGAVILGSTLLAGCSSNAKIDQGINPYVGFEMGYDWLGRMPYKGSVENGAYKAQGVQLTAKLGYPITDDLDIYTRLGGMVWRADTKSNVYGKNHDTGVSPVFAGGVEYAITPEIATRLEYQWTNNIGDAHTIGTRPDN

SEQ ID NO: 204 - protein, 305 aa, synthetic construct
  REGION 1..305: note = Synthetic: NGIgAsig anchor
PRAAQPRTQAAAQADAVSTNTNSALSDAMASTQSILLDTGAYLTRHIAQKSRADAEKNSVWMSNTGYGRDYASAQYRRFSSKRTQTQIGIDRSLSENMQIGGVLTYSDSQHTFDQAGGKNTFVQANLYGKYYLNDAWYVAGDIGAGSLRSRLQTQQKANFNRTSIQTGLTLGNTLKINQFEIVPSAGIRYSRLSSADYKLGDDSVKVSSMAVKTLTAGLDFAYRFKVGNLTVKPLLSAAYFANYGKGGVNVGGKSFAYKADNQQQYSAGVALLYRNVTLNVNGSITKGKQLEKQKSGQIKIQIRF

SEQ ID NO: 205 - protein, 201 aa, synthetic construct
  REGION 1..201: note = Synthetic: OsmY anchor
MTMTRLKISKTLLAVMLTSAVATGSAYAENNAQTTNESAGQKVDSSMNKVGNFMDDSAITAKVKAALVDHDNIKSTDISVKTDQKVVTLSGFVESQAQAEEAVKVAKGVEGVTSVSDKLHVRDAKEGSVKGYAGDTATTSEIKAKLLADDIVPSRHVKVETTDGVVQLSGTVDSQAQSDRAESIAKAVDGVKSVKNDLKTK

SEQ ID NO: 206 - protein, 232 aa, synthetic construct
  REGION 1..232: note = Synthetic: yiaT anchor
MLINRNIVALFALPFMASATASELSIGAGAAYNESPYRGYNENTKAIPLISYEGDTFYVRQTTLGFILSQSEKNELSLTASWMPLEFDPTDNDDYAMQQLDKRDSTAMAGVAWYHHERWGTVKASAAADVLDNSNGWVGELSVFHKMQIGRLSLTPALGVLYYDENFSDYYYGISESESRRSGLASYSAQDAWVPYVSLTAKYPIGEHVVLMASAGYSELPEEITDSPMIDR

SEQ ID NO: 207 - protein, 659 aa, synthetic construct
  REGION 1..659: note = Synthetic: intiminN anchor
MITHGCYTRTRHKHKLKKTLIMLSAGLGLFFYVNQNSFANGENYFKLGSDSKLLTHDSYQNRLFYTLKTGETVADLSKSQDINLSTIWSLNKHLYSSESEMMKAAPGQQIILPLKKLPFEYSALPLLGSAPLVAAGGVAGHTNKLTKMSPDVTKSNMTDDKALNYAAQQAASLGSQLQSRSLNGDYAKDTALGIAGNQASSQLQAWLQHYGTAEVNLQSGNNFDGSSLDFLLPFYDSEKMLAFGQVGARYIDSRFTANLGAGQRFFLPANMLGYNVFIDQDFSGDNTRLGIGGEYWRDYFKSSVNGYFRMSGWHESYNKKDYDERPANGFDIRFNGYLPSYPALGAKLIYEQYYGDNVALFNSDKLQSNPGAATVGVNYTPIPLVTMGIDYRHGTGNENDLLYSMQFRYQFDKSWSQQIEPQYVNELRTLSGSRYDLVQRNNNIILEYKKQDILSLNIPHDINGTEHSTQKIQLIVKSKYGLDRIVWDDSALRSQGGQIQHSGSQSAQDYQAILPAYVQGGSNIYKVTARAYDRNGNSSNNVQLTITVLSNGQVVDQVGVTDFTADKTSAKADNADTITYTATVKKNGVAQANVPVSFNIVSGTATLGANSAKTDANGKATVTLKSSTPGQVVVSAKTAEMTSALNASAVIFFDQTKAS

SEQ ID NO: 208 - protein, 656 aa, synthetic construct
  REGION 1..656: note = Synthetic: intiminN anchor
MITHGCYTRTRHKHKLKKTLIMLSAGLGLFFYVNQNSFANGENYFKLGSDSKLLTHDSYQNRLFYTLKTGETVADLSKSQDINLSTIWSLNKHLYSSESEMMKAAPGQQIILPLKKLPFEYSALPLLGSAPLVAAGGVAGHTNKLTKMSPDVTKSNMTDDKALNYAAQQAASLGSQLQSRSLNGDYAKDTALGIAGNQASSQLQAWLQHYGTAEVNLQSGNNFDGSSLDFLLPFYDSEKMLAFGQVGARYIDSRFTANLGAGQRFFLPANMLGYNVFIDQDFSGDNTRLGIGGEYWRDYFKSSVNGYFRMSGWHESYNKKDYDERPANGFDIRFNGYLPSYPALGAKLIYEQYYGDNVALFNSDKLQSNPGAATVGVNYTPIPLVTMGIDYRHGTGNENDLLYSMQFRYQFDKSWSQQIEPQYVNELRTLSGSRYDLVQRNNNIILEYKKQDILSLNIPHDINGTEHSTQKIQLIVKSKYGLDRIVWDDSALRSQGGQIQHSGSQSAQDYQAILPAYVQGGSNIYKVTARAYDRNGNSSNNVQLTITVLSNGQVVDQVGVTDFTADKTSAKADNADTITYTATVKKNGVAQANVPVSFNIVSGTATLGANSAKTDANGKATVTLKSSTPGQVVVSAKTAEMTSALNASAVIFFDGA

SEQ ID NO: 209 - protein, 5 aa, synthetic construct
  REGION 1..5: note = Synthetic: linker
GGGGS

SEQ ID NO: 210 - protein, 10 aa, synthetic construct
  REGION 1..10: note = Synthetic: linker
GGGGSGGGGS

SEQ ID NO: 211 - protein, 15 aa, synthetic construct
  REGION 1..15: note = Synthetic: linker
GGGGSGGGGSGGGGS

SEQ ID NO: 212 - protein, 20 aa, synthetic construct
  REGION 1..20: note = Synthetic: linker
GGGGSGGGGSGGGGSGGGGS

SEQ ID NO: 213 - protein, 5 aa, synthetic construct
  REGION 1..5: note = Synthetic: linker
EAAAK

SEQ ID NO: 214 - protein, 10 aa, synthetic construct
  REGION 1..10: note = Synthetic: linker
EAAAKEAAAK

SEQ ID NO: 215 - protein, 15 aa, synthetic construct
  REGION 1..15: note = Synthetic: linker
EAAAKEAAAKEAAAK

SEQ ID NO: 216 - protein, 8 aa, synthetic construct
  REGION 1..8: note = Synthetic: linker
GGGGGGGG

SEQ ID NO: 217 - protein, 6 aa, synthetic construct
  REGION 1..6: note = Synthetic: linker
GGGGGG

SEQ ID NO: 218 - protein, 46 aa, synthetic construct
  REGION 1..46: note = Synthetic: linker
AEAAAKEAAAKEAAAKEAAAKALEAEAAAKEAAAKEAAAKEAAAKA

SEQ ID NO: 219 - protein, 17 aa, synthetic construct
  REGION 1..17: note = Synthetic: linker
VSQTSKLTRAETVFPDV

SEQ ID NO: 220 - protein, 5 aa, synthetic construct
  REGION 1..5: note = Synthetic: linker
PAPAP

SEQ ID NO: 221 - protein, 12 aa, synthetic construct
  REGION 1..12: note = Synthetic: linker
AEAAAKEAAAKA

SEQ ID NO: 222 - DNA, 45 bp, synthetic construct
  misc_feature 1..45: note = Synthetic: scFvLinker
  source 1..45: mol_type = other DNA
ggtggaggcggctctggcggtggtgggagtggaggcggtggcagt

SEQ ID NO: 223 - protein, 34 aa, synthetic construct
  REGION 1..34: note = Synthetic: linker
  VARIANT 1..34: note = This sequence may encompass 5-17 "AP"
APAPAPAPAPAPAPAPAPAPAPAPAPAPAPAPAP

SEQ ID NO: 224 - protein, 6 aa, synthetic construct
  REGION 1..6: note = Synthetic: linker
PLGLWA

SEQ ID NO: 225 - protein, 6 aa, synthetic construct
  REGION 1..6: note = Synthetic: linker
RVLAEA

SEQ ID NO: 226 - protein, 10 aa, synthetic construct
  REGION 1..10: note = Synthetic: linker
EDVVCCSMSY

SEQ ID NO: 227 - protein, 9 aa, synthetic construct
  REGION 1..9: note = Synthetic: linker
GGIEGRGSC

SEQ ID NO: 228 - protein, 10 aa, synthetic construct
  REGION 1..10: note = Synthetic: linker
TRHRQPRGWE

SEQ ID NO: 229 - protein, 10 aa, synthetic construct
  REGION 1..10: note = Synthetic: linker
AGNRVRRSVG

SEQ ID NO: 230 - protein, 9 aa, synthetic construct
  REGION 1..9: note = Synthetic: linker
RRRRRRRRR

SEQ ID NO: 231 - protein, 4 aa, synthetic construct
  REGION 1..4: note = Synthetic: linker
GFLG

SEQ ID NO: 232 - protein, 28 aa, synthetic construct
  REGION 1..28: note = Synthetic: linker
  VARIANT 1..28: note = This sequence may encompass 1-4 "GGGSGGG" repeating units
GGGSGGGGGGSGGGGGGSGGGGGGSGGG

SEQ ID NO: 233 - protein, 20 aa, synthetic construct
  REGION 1..20: note = Synthetic: linker
  VARIANT 1..20: note = This sequence may encompass 1-4 "GGSGG" repeating units
GGSGGGGSGGGGSGGGGSGG

SEQ ID NO: 234 - protein, 12 aa, synthetic construct
  REGION 1..12: note = Synthetic: linker
  VARIANT 1..12: note = This sequence may encompass 1-4 "GSG" repeating units
GSGGSGGSGGSG

SEQ ID NO: 235 - protein, 24 aa, synthetic construct
  REGION 1..24: note = Synthetic: linker
  VARIANT 1..24: note = This sequence may encompass 1-4 "GGGSGG" repeating units
GGGSGGGGGSGGGGGSGGGGGSGG

SEQ ID NO: 236 - protein, 18 aa, synthetic construct
  REGION 1..18: note = Synthetic: linker
KESGSVSSEQLAQFRSLD

SEQ ID NO: 237 - protein, 14 aa, synthetic construct
  REGION 1..14: note = Synthetic: linker
EGKSSGSGSESKST

SEQ ID NO: 238 - protein, 12 aa, synthetic construct
  REGION 1..12: note = Synthetic: linker
GSAGSAAGSGEF

SEQ ID NO: 239 - DNA, 24 bp, synthetic construct
  misc_feature 1..24: note = Synthetic: UP element
  source 1..24: mol_type = other DNA
ggaaaatttttttaaaaaaaaaac

SEQ ID NO: 240 - protein, 173 aa, synthetic construct
  REGION 1..173: note = Synthetic: PAL
MQLNKVLKGLMIALPVMAIAACSSNKNASNDGSEGMLGAGTGMDANGGNGNMSSEEQARLQMQQLQQNNIVYFDLDKYDIRSDFAQMLDAHANFLRSNPSYKVTVEGHADERGTPEYNISLGERRANAVKMYLQGKGVSADQISIVSYGKEKPAVLGHDEAAYAKNRRAVLVY

SEQ ID NO: 241 - DNA, 28 bp, synthetic construct
  misc_feature 1..28: note = Synthetic: RBS
  variation 13..15: note = n is a, c, t, g, unknown or other
  variation 18..20: note = n is a, c, t, g, unknown or other
  source 1..28: mol_type = other DNA
tctagagaaagannngannnactagatg

SEQ ID NO: 242 - DNA, 28 bp, synthetic construct
  misc_feature 1..28: note = Synthetic: RBS
  source 1..28: mol_type = other DNA
tctagagaaagaggggacaaactagatg

SEQ ID NO: 243 - DNA, 28 bp, synthetic construct
  misc_feature 1..28: note = Synthetic: RBS
  source 1..28: mol_type = other DNA
tctagagaaagacaggacccactagatg

SEQ ID NO: 244 - DNA, 28 bp, synthetic construct
  misc_feature 1..28: note = Synthetic: RBS
  source 1..28: mol_type = other DNA
tctagagaaagatccgatgtactagatg

SEQ ID NO: 245 - DNA, 28 bp, synthetic construct
  misc_feature 1..28: note = Synthetic: RBS
  source 1..28: mol_type = other DNA
tctagagaaagattagacaaactagatg

SEQ ID NO: 246 - DNA, 28 bp, synthetic construct
  misc_feature 1..28: note = Synthetic: RBS
  source 1..28: mol_type = other DNA
tctagagaaagaagggacagactagatg

SEQ ID NO: 247 - DNA, 28 bp, synthetic construct
  misc_feature 1..28: note = Synthetic: RBS
  source 1..28: mol_type = other DNA
tctagagaaagacatgacgtactagatg

SEQ ID NO: 248 - DNA, 28 bp, synthetic construct
  misc_feature 1..28: note = Synthetic: RBS
  source 1..28: mol_type = other DNA
tctagagaaagataggagacactagatg

SEQ ID NO: 249 - DNA, 28 bp, synthetic construct
  misc_feature 1..28: note = Synthetic: RBS
  source 1..28: mol_type = other DNA
tctagagaaagaagagactcactagatg

SEQ ID NO: 250 - DNA, 28 bp, synthetic construct
  misc_feature 1..28: note = Synthetic: RBS
  source 1..28: mol_type = other DNA
tctagagaaagacgagatatactagatg

SEQ ID NO: 251 - DNA, 28 bp, synthetic construct
  misc_feature 1..28: note = Synthetic: RBS
  source 1..28: mol_type = other DNA
tctagagaaagactggagacactagatg

SEQ ID NO: 252 - DNA, 28 bp, synthetic construct
  misc_feature 1..28: note = Synthetic: RBS
  source 1..28: mol_type = other DNA
tctagagaaagaggcgaattactagatg

SEQ ID NO: 253 - DNA, 28 bp, synthetic construct
  misc_feature 1..28: note = Synthetic: RBS
  source 1..28: mol_type = other DNA
tctagagaaagaggcgatacactagatg

SEQ ID NO: 254 - DNA, 28 bp, synthetic construct
  misc_feature 1..28: note = Synthetic: RBS
  source 1..28: mol_type = other DNA
tctagagaaagaggtgacatactagatg

SEQ ID NO: 255 - DNA, 28 bp, synthetic construct
  misc_feature 1..28: note = Synthetic: RBS
  source 1..28: mol_type = other DNA
tctagagaaagagtggaaaaactagatg

SEQ ID NO: 256 - DNA, 28 bp, synthetic construct
  misc_feature 1..28: note = Synthetic: RBS
  source 1..28: mol_type = other DNA
tctagagaaagatgagaagaactagatg

SEQ ID NO: 257 - DNA, 28 bp, synthetic construct
  misc_feature 1..28: note = Synthetic: RBS
  source 1..28: mol_type = other DNA
tctagagaaagaagggatacactagatg

SEQ ID NO: 258 - DNA, 28 bp, synthetic construct
  misc_feature 1..28: note = Synthetic: RBS
  source 1..28: mol_type = other DNA
tctagagaaagacatgaggcactagatg

SEQ ID NO: 259 - DNA, 28 bp, synthetic construct
  misc_feature 1..28: note = Synthetic: RBS
  source 1..28: mol_type = other DNA
tctagagaaagacatgagttactagatg

SEQ ID NO: 260 - DNA, 28 bp, synthetic construct
  misc_feature 1..28: note = Synthetic: RBS
  source 1..28: mol_type = other DNA
tctagagaaagagacgaatcactagatg

SEQ ID NO: 261 - DNA, 28 bp, synthetic construct
  misc_feature 1..28: note = Synthetic: RBS
  source 1..28: mol_type = other DNA
tctagagaaagatttgatatactagatg

SEQ ID NO: 262 - DNA, 28 bp, synthetic construct
  misc_feature 1..28: note = Synthetic: RBS
  source 1..28: mol_type = other DNA
tctagagaaagacgcgagaaactagatg

SEQ ID NO: 263 - DNA, 28 bp, synthetic construct
  misc_feature 1..28: note = Synthetic: RBS
  source 1..28: mol_type = other DNA
tctagagaaagagacgagtcactagatg

SEQ ID NO: 264 - DNA, 28 bp, synthetic construct
  misc_feature 1..28: note = Synthetic: RBS
  source 1..28: mol_type = other DNA
tctagagaaagagaggagccactagatg

SEQ ID NO: 265 - DNA, 28 bp, synthetic construct
  misc_feature 1..28: note = Synthetic: RBS
  source 1..28: mol_type = other DNA
tctagagaaagagatgactaactagatg

SEQ ID NO: 266 - DNA, 28 bp, synthetic construct
  misc_feature 1..28: note = Synthetic: RBS
  source 1..28: mol_type = other DNA
tctagagaaagagccgacatactagatg

SEQ ID NO: 267 - DNA, 28 bp, synthetic construct
  misc_feature 1..28: note = Synthetic: RBS
  source 1..28: mol_type = other DNA
tctagagaaagagccgagttactagatg

SEQ ID NO: 268 - DNA, 28 bp, synthetic construct
  misc_feature 1..28: note = Synthetic: RBS
  source 1..28: mol_type = other DNA
tctagagaaagaggtgactcactagatg

SEQ ID NO: 269 - DNA, 28 bp, synthetic construct
  misc_feature 1..28: note = Synthetic: RBS
  source 1..28: mol_type = other DNA
tctagagaaagagtggaactactagatg

SEQ ID NO: 270 - DNA, 28 bp, synthetic construct
  misc_feature 1..28: note = Synthetic: RBS
  source 1..28: mol_type = other DNA
tctagagaaagataggactcactagatg

SEQ ID NO: 271 - DNA, 28 bp, synthetic construct
  misc_feature 1..28: note = Synthetic: RBS
  source 1..28: mol_type = other DNA
tctagagaaagattggacgtactagatg

SEQ ID NO: 272 - DNA, 28 bp, synthetic construct
  misc_feature 1..28: note = Synthetic: RBS
  source 1..28: mol_type = other DNA
tctagagaaagaaacgacatactagatg

SEQ ID NO: 273 - DNA, 28 bp, synthetic construct
  misc_feature 1..28: note = Synthetic: RBS
  source 1..28: mol_type = other DNA
tctagagaaagaaccgaattactagatg

SEQ ID NO: 274 - DNA, 28 bp, synthetic construct
  misc_feature 1..28: note = Synthetic: RBS
  source 1..28: mol_type = other DNA
tctagagaaagacaggattaactagatg

SEQ ID NO: 275 - DNA, 28 bp, synthetic construct
  misc_feature 1..28: note = Synthetic: RBS
  source 1..28: mol_type = other DNA
tctagagaaagacccgagacactagatg

SEQ ID NO: 276 - DNA, 28 bp, synthetic construct
  misc_feature 1..28: note = Synthetic: RBS
  source 1..28: mol_type = other DNA
tctagagaaagaccggaaatactagatg

SEQ ID NO: 277 - DNA, 28 bp, synthetic construct
  misc_feature 1..28: note = Synthetic: RBS
  source 1..28: mol_type = other DNA
tctagagaaagaccggagacactagatg

SEQ ID NO: 278 - DNA, 28 bp, synthetic construct
  misc_feature 1..28: note = Synthetic: RBS
  source 1..28: mol_type = other DNA
tctagagaaagagctgagcaactagatg

SEQ ID NO: 279 - DNA, 28 bp, synthetic construct
  misc_feature 1..28: note = Synthetic: RBS
  source 1..28: mol_type = other DNA
tctagagaaagagtagatcaactagatg

SEQ ID NO: 280 - DNA, 28 bp, synthetic construct
  misc_feature 1..28: note = Synthetic: RBS
  source 1..28: mol_type = other DNA
tctagagaaagatatgaataactagatg

SEQ ID NO: 281 - DNA, 28 bp, synthetic construct
  misc_feature 1..28: note = Synthetic: RBS
  source 1..28: mol_type = other DNA
tctagagaaagattagagtcactagatg

SEQ ID NO: 282 - DNA, 31 bp, synthetic construct
  misc_feature 1..31: note = Synthetic: RBS
  source 1..31: mol_type = other DNA
tctagagttcacacaggaaacctactagatg

SEQ ID NO: 283 - DNA, 31 bp, synthetic construct
  misc_feature 1..31: note = Synthetic: RBS
  source 1..31: mol_type = other DNA
tctagagattaaagaggagaaatactagatg

SEQ ID NO: 284 - DNA, 30 bp, synthetic construct
  misc_feature 1..30: note = Synthetic: RBS
  source 1..30: mol_type = other DNA
tctagagtcacacaggaaacctactagatg

SEQ ID NO: 285 - DNA, 29 bp, synthetic construct
  misc_feature 1..29: note = Synthetic: RBS
  source 1..29: mol_type = other DNA
tctagagtcacacaggaaagtactagatg

SEQ ID NO: 286 - DNA, 27 bp, synthetic construct
  misc_feature 1..27: note = Synthetic: RBS
  source 1..27: mol_type = other DNA
tctagagtcacacaggactactagatg

SEQ ID NO: 287 - DNA, 28 bp, synthetic construct
  misc_feature 1..28: note = Synthetic: RBS
  source 1..28: mol_type = other DNA
tctagagaaagaggagaaatactagatg

SEQ ID NO: 288 - DNA, 30 bp, synthetic construct
  misc_feature 1..30: note = Synthetic: RBS
  source 1..30: mol_type = other DNA
tctagagattaaagaggagaatactagatg

SEQ ID NO: 289 - DNA, 28 bp, synthetic construct
  misc_feature 1..28: note = Synthetic: RBS
  source 1..28: mol_type = other DNA
tctagagaaagaggggaaatactagatg

SEQ ID NO: 290 - DNA, 25 bp, synthetic construct
  misc_feature 1..25: note = Synthetic: RBS
  source 1..25: mol_type = other DNA
ttatataaaagtgggaggtgcccga

SEQ ID NO: 291 - DNA, 72 bp, synthetic construct
  misc_feature 1..72: note = Synthetic: RBS
  source 1..72: mol_type = other DNA
gaccagaggtaaggaggtaacaaccatgcgagtgttgaagaaacatcttaatcatgctggggagggtttcta

SEQ ID NO: 292 - DNA, 9687 bp, synthetic construct
  misc_feature 1..9687: note = Synthetic: Phage 3 DNA E. coli
  source 1..9687: mol_type = other DNA
aatcgccggtgtactccgcgtcagaaaggtatacagccacggcagggagatcctgctcttcaagaaaaacagggcgcccgtcaaaccaggtgaccgtgtcggtgatctcggctttcagtttggccagaatggctgcacgaattgcgctgtgtctgttcatcgcttcaggtggatcctcagttggtttttcagggctgcggaaagttctttgggcatatcgctttcaataaggcgctttgaaatagcggtgaaggccacggtgagcggtgtctcaagaggaactttgaccacatcaatcggataacgggcctgacctacgcgccgcatgacctgccagcgcccgttcgcaagctgttggataaaagcgttacgaaaggtatagggcccgattttaaggacgctgcccgctccgtttctggccccttttttacgcgagagcctgacgcgcgccgtgccgagctttatcgcaggaagattaccgcggttgatttttatcgacgcgaccgggcgatcgtgacgggccttgcgcagacgggaacgctggcggaccagacgaaccggaagcccctttttccggttatcatcaactgttgcttctttcgctacagctttgctcccctggcttatcgttcttctggccaccctgttaagtgcttttgcggttgcctcaggaacgattaaccggctgaggctgttcaggttctgaatagccctttccagtcctttcacagacatagcgcctcctcattcgagatggatgcggggttttccgttgaacatgtcatagcgggtaacgatcaggttcttaccgtcgtagtcgacgctgtcgtttcggcgtggctggtaaagctcagagaaaaccaccagcgaagtacctgttcccgacaatggccccatttcctcgagttgctcggcgggaacaacgtcatagctgctgccattgatgatcgctgtctttcccatcttttttatagtggccgcgtccatgcgcgccgccatccggtcaaaggagttaggcattgatcttaacttcaacaacggtggtgtttgcccctgcatcttcccaggcgatgcccgcggcaacggcgtccgtttcttcgatcgtgattttgccgtccttcagatacacctgcgccccggcagtaaccgcatctgcggatacttttggcaggaggaaaacaccctcagtaaaaccgtccccggtatcgccagccgggatatcggtaattgccaccgcgataagttttccaacaacaaccgggtcgccgctgtgaacatcggttgcaccactgtttaccagagggatcgttttcccgtcctgcgcatagttcttagccataacttctccattcagcccctttcgaggctggtttcaggtataaaaaaagcccttacgggcgtctgtttgtcaggactgttttttactgaccagaggatttggtcatgccgcgatagtccagcggcgccacgccagcatcaatacgcactttcgtggcgataccatcagtggtgaagccttcctgctgatcgatgtatggcgtgtcgacgccgttgagataagcgacctcaatggtgtcggtgcccttcgcggcagccagataccaggctttcgcatcagcttcatccagacgtggttcggcaatgacttctgcaaagttctggatagggttaacgatcccggcattgatgtctgcacctttaacactggccgacttgatggtctgatttgccagagtttccagggcgacgggcaccagcatgtaggccggacggatattcagggttcgctccccctccttctgcagacgcatcagcttgcgcgattcgtccaggctggccacagaaattgcacccgagctcaggttcttgtgatcggcatggaacagcgcctttccgtctgagagtttcgggtttttggtcagaatggcgtaaaccagatcgccaatcgttgctttcgccgcgcgccccatcttcatcggtacgtcggtaagctggttcagatcgtcgttgatgatcgcctggcgagttactgagaagatttcaccatacgtggcaagcgcgatggtttcgcctttgtcactggtagtgatgtacttgtactcagccccttcgcgaacctgtcgcagagaagggaacccacccataccgacacgatgcgccgttttgaagtccgacagctggccttttttggtccactgctcgaaggtttcctgcgcctcgtcccagccctgaatcagcgctttgttcgcaacatcaagcagaatgttgccaaagtcagaggtgctgtgggtcagcgccaggccaaccatctgcatcgggttgtagctggccacgccgatacctttttctgtcagggccatacgcgcatactcgcgcagcgtcataccgttataaacgttatcccgctcctgaccttcgaacccggcacgcgccatcagtgcctggcgaataccatccgcgacgaagttaccgttgcccgcatgaatatgcggctgagtggttttattggacggcgtggccgttttaccgagttctgccagcagcaaatctttcgccttatcgacggagcaatcagggtcggccacacactgattctgcagttccatgtgcttattaccgaacatggcaaagagatcgccgatagcgttaacacgggttttctgctcagccaacacctgcgcgcggatcgcattttcatccggtgccgggtctgtttttgcctgcggtgcctgaggctgggtaataaccgggtcacgctgggtagtgttgcgcggcggggtgatcatgttgcgaatgctttttggcattttttcaaattcctcaatacgttttgaatgaatacaggccatagcctgaagggatggtgtcacctggtcggcaaaacccagttcaaggcactcgctgccgttcatccaggtttcgtcctccagcattaccgcaatttcttcggtggattttccggttttctgtgcataagccgggataagaacggattcaaccttgtcgagaagatccgcatagtcgcgcatatcgctcgcgtcaccaccagcaaacccccagggcttatggatcatcatcatcgtgttttcaggcatgatgaccggattgcctaccatcgcaatcaccgaggccatggaggccgccagaccgtcgatatgtacggtaatcgccgcgccgtggtgcttcagcgcgttataaatagcaattccgtcgaagacatcaccaccgggcgagttgatataaaggttgatgtgggtgacgtccccaagtgcccggagatcattgacgaactgtttcgccgttacgccccagtacccgatttcgtcataaataaaaatgtcggcctcactgttattgctggcctgcatgcggaaccacgaattactttttgcgctggctttcggacggtggcgcgcccggttctttggcttcggcactggtgcctcctttatcattggcggggtcggtgtcaaacaccaggccctgttcacggttctcgtcaacctcagctttacggcgtgacttaacatcatccgggttgcgaccgctggcacgtatccagtcggattcagtagcagcaccgccgcggatctgcgttttccaggcattcgcttctttaacgggatcaatccacggcataacgggccccgaataaaccgcgttataaagcgagtccatatcaatgcctctcggcagcttgatttctccggcagcaatagccatcttgagccaggctcggtacatgggccgggtcactgaaccgatgaaccagtcctgaagaatcagatagccgtcggttgactcgacaagctcctgccgctgggcactgtacgttccgttgtagtttctggatgtgctggaaaagctgaggcgactgccggcggacacggcacgcagctgtccgttacgaaaagattcgaggttagggttcgggcgatcggatttaatcatcccgatttcttccccggcctgcagttcgtcatagagcataccgggctgaatcatcagctcgcggtcatcgctgcttgaatcagaatcgaagctctgtccgtcgccttttttgatatacatgccgagtgccgcagcaattctggcagcagtaagctccgagtcctcgtattctttcagcgcgctcagacgcatcagaacaccagacaaaagagacgttccgcgggtctggtgcaggcgtcgggtgaatttgagatgcagcatgttctctgcatctatctctttggtatcaaactgacgcccggatactggcaggcttttatagacctgatattttttcgggcgtccccagttatcgacaaaaacgccctgattgagctgggtggcagcatcgctgttcatcggcacaaagtccggctccagcgcttccagccagaacggcacgccagcaaccggctgaagaccatttccggtaccgcgaaccagctgagcaaatacctcaccgtcccggagccacgttcgcagcatcagccgctccagcattgggcgggtaaactgggttgtgacatctggccttacggaccattcgccccactttcggcggatatcagtggccagctttttagcgatcttcccgttactcagcatcggatgcggttcaactatgatgcccttcgcacccaccaccctttcttccagcttgtcgaaaacgccgatcaccagatcgtggttgttatccagccagcgcgcctgctgcctcagcgaaaccgcccccatctggctgagctgatcggctgaacgattttccttctgggctttgtgggtacgcgtttgctttaccgcctcatacgctttaataactgcgcgggcacgcaggcgtgaggctttccagcctggtgaaaacaggccaatcgcatcatctaaaaaactcatccaaacctcgccagcctgtagccgggtcgcccacggcgtttgttattgagcgttgccagtcgtcgctcccattcctgacggccttttctgatttccgacaggttttcgagcgtcatctgctgcccgttgaaagtgattgatttcccctccagaacagacagctcggctgcagcatagcggtcgatcatgttttgaatatctgctggattcacacccaacctcctgacgaagaccacggattagcctgctcggttacgggcttctcacgttttggttttggtttagatttcggcgcaggcggcggggatggcatttcgccagcttccgtctgcgtgtcctcgatccacgtttcccgccgtgcccactcaggagctgacggccatttgattttttcgtaaccactaaggatggcgagcgcgtcggcataaacgagcaggtcaaatgcttcgtttgcgccccggccgggcttactccatttcccttcattcgagcgttcctcatacgtcagttcgtcatagaaccagctgcccagccaggcggggaaatgcacatagccagggccgggtgaatcacgccacagcgcattattcacccggtctttaagggcatcggtctggagaagataaagaggcacatcaccagtcgcctgtgcgcggcgcgttgatctgcccgtgttgtcgggaaacgttcgctggataagtttgctgcgcctgacgctgtcccctttgaagagatagatacgcttacccagcccctcacggcgacatctgcgccagaacttgtaggcattatccgtcacgccatcttcgccccctgagtccacggccatcgacatcagccgcatgccctttgatgggtcagctgcgagcggccacgttttatcaaagacgtcagtgagtaaaagatcccagtcctccggatagctcgccggatccacctgaatgctttcaccgttgccgtcgcagcgcagcgaatgccggatgttgtaacggtcaactatccagcgctcacccatacttccataacccgtaatctgcacaacaaagcgccggttgcgcccggcctgcacgtccacggtcgcagtgagaaactgcacgccgttcggtaccgaacgttttgggacgtcttcggcacgctgctcgagcaattcacttttacgctgctccatgctggctcgcggcaaatagggcctgccgaaatcggtgttgatcaccgtcttcagggtttcttcgctgcgcgtggattcatattcctgctcggcggtcagaaacttataaataagctgcgcccaggtctggtaagcagctgccggaccttccatccagaaggaggcaatacgggaacgacggccatcaccgctaaccaggcctttcctgtcgatggtttgcccgtcccggagccagacacatttcatgttaagcgcacgcttcatgtccggtgtgatcctgcctttacaggcagggcactgaagaaacgccgcttcgctggcaagcacaggatcgctgctgtcgcggtatccggtcatattgtccatttccggctggaaatattcgccgcaatgcgggcatggccagtaaagacgacggcggtcaccacggttatagagcgataaaattccggtggtcggaggggcttcatggggcgtggagcgccgccattttgtgtctctgatatccctcccgggcgagctctcaaccagcgtcatcccggaggacatgaatgtcgtggttcgtttcgatgccagtgaaaaagcatccccctccccgtcgatatcttccggaaagcggtcataatccgtcagcgccacacttttatagtccgaggacgacatgatattgacggatggccagcccagcttcagatagttaccggcgcggaatgtacggtcgtagacgttgttatcgttacgtcttgggcttagccgggttttaacttcagggctacagcgaaaagtacggtccaggcgttttttggaatgctcgcgcgctttttcctcagatacctgaattacaagcatatctgccggatcgcagacaatgttataaacgatccagccgtcaatcagcccgatggttttacccgttcgcgctgggcccacaaacacaaccgcatcgtattcacgcgatgccagacagttcatcggctcaatcacatagggtgccagatccggatcccacggaactgagtttcccgcccccattggcacgcgcatataagtactgaccgcatcggccaccggcatacgacgcggggctcgtaaaataccggaaacatcgcggcggatgtccctggcggatgcccgctttgccatcagtcctcctcaggctgctcctcctcttttccagcgtcctgcaccttctccgccatctggtcgcgcagatcatcgataacgctttgcacacgaactaccgcagcaggcgttaaagcacagtcgcgctcgagcacatccgggagggtttcaagtaccatgacgacggctttcgccatcaatgagaattctcgcgccacttcatctgcgggtattaactgccccgtatcctgttcgaacttcagcctctcgttctctgctttccagtgggacagcctgtcagaagggggcatatcgtcgatgttggccgaaacggtagggatcatcagttcggtcagaatgtcggtcaccagatagagctttaacttgctattgctgcctggagcaggttcaacatttttcagtctcgcggcaaccgtctgacggtgtacgccggttatccctgccagctggttgatattgagttttaaagtggcaatttcctggtccatgatggtgaacactttttgaacgattcgacatgttgcgaaaatggcctctaattaaatcaaagacctgcgcacatgatgatgatgaccctggatccgaaaaactagccgtttcccgcgagcacgccgccccgtggcagggtccccctccgggagtaccttttgataataattatcaattgcacactatcgacggcactgctgccagataacaccaccggggaaacattccatcatgatggccgtgcggacataggaagccagttcatccatcgctttcttgtctgctgccatttgctttgtgacatccagcgccgcacattcagcagcgtttttcagcgcgttttcgatcaacgtttcaatgttggtatcaacaccaggtttaactttgaacttatcggcactgacggttaccttgttctgcgctggctcatcacgctggataccaaggctgatgttgtagatattggtcaccggctgaggtgtttcgattgccgctgcgtggatagcaccatttgcgatagcggcgtccttgatgaatgacactccattgcgaataagttcgaaggagacggtgtcacgaatgcgctggtccagctcgtcgattgccttttgtgcagcagaggtatcaatctcaacgccaagcgtcatcgaagcgcaatattgctgctcaccaaaacgcgtattgaccaggtgttcaacggcaaatttctgcccttctgatgtcagaaaggtaaagtgattttctttctggtattcagttgctgtgtgtctggtttcagcaaaaccaagctcgcgcaattcggctgtgccagatttagaaggcagatcaccagacagcaacgcgccacggaaaaacagcgcataaagcacttcattagcagcgccagatagcgtaatgattttgttactcatggaatatttccttttaggcgtgagcctgtcgcacggcaatgccgcccgagaggtaaacgcaacctaacggcatcacccaggctcactactgaaagactctctttgatgtgcgcgtgcgatgcgcgtagaagactgatttatcaacctgtctttatatcaggattcattacctgactatttgtgggtaaagttcgtagtgcgctgatcgtgcaaaatgattttagttgggaacagttcgcaactctgtcccataaaaatcagcatattcccatctatcccatatccagcgcattgaccatcgggatactgaagggagattccatcatctcttagaaagatcaccatctcttttgtttcaatttgcatatagctacctggaggatttatgaatgcaaggattttcatggactattaccatgagattgattttccatctttattcgcgagagcagtggaaagcgatgacgatgtgggtactacattgcgcattcacctactttgtgagcgcatggtcgaagcatggatatgcgcatgctgtgactgccaagatctctttggaagagataaaaacaaacttttaatcgaatgtaatactaaaatatccatggcgggaaacctgggaatccccccggaacttatgaaatcacttaaaaccatcaactcaatgcgtaatgaccttgcacacaatccatcaatacaaagcattgctgattcaaggatccagagcctgaaggatactctgactgaatactttaaacagcatccaacggaacccagcatggaagaatcaaaactgggtatttttaacgccgagaatcaattaaccgaagaagtttccttagatagtgacagttcaaaaaacagacttaagttaatcttgctgttcagcaagttaatgcaggcgttaatgcaattagttgcagctaatcataatgggcgctgggataaccaatttagccaattcgtttaccatgtgaccatgaacgcaacaaagagataaatccaagcccgttttgtacgggctgttgcattatcacaggcactcagtgaatgcctgctgtaatgccgctagtcgtcgagttgcaacacaccgtg

SEQ ID NO: 293 - DNA, 424 bp, synthetic construct
  misc_feature 1..424: note = Synthetic: clbA knockout sequence
  source 1..424: mol_type = other DNA
ggatggggggaaacatggataagttcaaagaaaaaaacccgttatctctgcgtgaaagacaagtattgcgcatgctggcacaaggtgatgagtactctcaaatatcacataatcttaacatatcaataaacacagtaaagtttcatgtgaaaaacatcaaacataaaatacaagctcggaatacgaatcacgctatacacattgctaacaggaatgagattatctaaatgaggattgatgtgtaggctggagctgcttcgaagttcctatactttctagagaataggaacttcggaataggaacttcggaataggaactaaggaggatattcatatgtcgtcaaatgggcagaattgaatcgccacggataatctagacacttctgagccgtcgataatattgattttcatattccgtcggtgg

SEQ ID NO: 294 - DNA, 735 bp, synthetic construct
  misc_feature 1..735: note = Synthetic: clbA
  source 1..735: mol_type = other DNA
atgaggattgatatattaattggacatactagtttttttcatcaaaccagtagagataacttccttcactatctcaatgaggaagaaataaaacgctatgatcagtttcattttgtgagtgataaagaactctatattttaagccgtatcctgctcaaaacagcactaaaaagatatcaacctgatgtctcattacaatcatggcaatttagtacgtgcaaatatggcaaaccatttatagtttttcctcagttggcaaaaaagattttttttaacctttcccatactatagatacagtagccgttgctattagttctcactgcgagcttggtgtcgatattgaacaaataagagatttagacaactcttatctgaatatcagtcagcatttttttactccacaggaagctactaacatagtttcacttcctcgttatgaaggtcaattacttttttggaaaatgtggacgctcaaagaagcttacatcaaatatcgaggtaaaggcctatctttaggactggattgtattgaatttcatttaacaaataaaaaactaacttcaaaatatagaggttcacctgtttatttctctcaatggaaaatatgtaactcatttctcgcattagcctctccactcatcacccctaaaataactattgagctatttcctatgcagtcccaactttatcaccacgactatcagctaattcattcgtcaaatgggcagaattga

SEQ ID NO: 295 - DNA, 9621 bp, synthetic construct
  misc_feature 1..9621: note = Synthetic: clbB
  source 1..9621: mol_type = other DNA
atggataatacctctggagattttccatgtaataagatggacacgcgtaagcagttaccgctaacaccaagtcaacaggggtttttattccattccttaaaggataagaaaaggagtaactaccatgagcattttacatgcattttttctcagcatgtagatagcgcccacttcaagtgggcgctggaaacgttatttcgaaagcatgagtgttttcgcactgattataactgggagattgatgagcgcccttgtcaggtggtgaagaccgatgtgttgccggatatatatgtgttagactgtgagcaagaggaaatacgttttctactagctaatgatgacattatcattcctgtcccgcaggatgacggtattgatgctataattcctcaactgctacaggctgatttaaaatacccattttccttgaaaacgatcccagtccgggcctaccttattcagtcaacgaaagaaagtgcttttatactatcataccatcatattgtgatggatggctggagcttatcccttttcattaaacagttgctccaactctatggagcggctgtggtcagtggtgtgagggatgatagcgccattatcccctcatctctgaaaccccttgtagacacactgtcggcccgacgtcacacctttcagcacgactattgggctgcatatcttcgggagggaacaccaacttgtatcgtgccgctgtcacaatatcacacagatactgaagccgagaacaattcttacgttaatcaaacaaatcatgtggagatcaatttgtctccggatgtgtgtcagaaaatacagacgctatgcagcgattatcgtatcacccccgcagtaatcttctatgtggcctggggcatcctgctacaacgttggtgctatgctgacgatgtgttattcggcgcgacaatatcagggcgaaatataccaattgatggtatagaagaaacactagggctatttattaacacgttgccgctgcgtctgcgtgatgatggggcgacactgttgcaacacttacaacgtatgcaccaaacactaatagctcactacagcaatgaacatgatgccttagccagcatacaacggttggtacataaagaaggtcatgctggggatctttttaataccttagtggtgttggagaattatcccgttgatatgacattattgtcatgcgcgtcgcccgtggcaattcgccatctcagtgtacatgaacaaacgcactatccgctgaccttgaccatcactcaacagaagggattccgtttcagtattgcatatgcccttaactacctgaccaacaacatggcgcaagcgttgctgatgcacctgagttatctgcttgaacaactggtggacaatccgcagcgccccattgctgcgttggtaaacttgtccccatgccagcaggcgcaggtacttcaaccctatctggaacgcatggcatgccgggattgggatagtcaatccaacgtcatcgaacaatttcatcaagttgctgcgacttcgccagcacaggtcgcagtggttgatgaattgtgcgcgttgacctattcggagttggcagcacaagctgagcagctcgcggcttatttggtacagcagggtgttatggttggcgatactgtaggcataattagcgaacgccgggtaaacaccgtggttgccatcatcgccatcatgttgatcggggctgcttatgtgcccatcagccccgactacccagtgggtaggatgcaggaaattattgatgacagcggcttggcgctgttgctggtacatggcaaaccgctagatgcattaaacgttgcgcagagtgacctctgtgcatttcccgtcgcgccctcggtggtatttccggttatcacaccagattctcgcgcttatgtgatttattcatcggggtcgacgggtaagccaaaaggtatcgcggtggcgcaccgcggtttattgcgcctgatacaaggcgacagcccgctgaaggtggagagcggtgagacaacgctgctgacctgcccatttgagtttgatgtttcggtgtttgagatgtggtctaccttgctcaaccacggcaaactagtattactcagcaaacaggcattacttgatatcaatcacattcgccgcacgatcgctgatgaacaggtggcgcgcgcctggtttacctcatccttgtttaactcctatgtggcagaaggtgccgatttcttcggtatgttacaacacattacggtgggcggcgaagcagtatctgcgtggcatgtcaacgacgtgatgcaaaaatacccgcatctggtggtgacaaacggttatgggccgacggaaaacactatttttaccaccgcatatcgtttcaacgggttgcaacccgcccgagtcccgataggatacgcggtaccgggcacctcgctctacattaccgatctccatgggcatttgttgcctatcggtgccaccggtgaactagtggcgggtggagtgggggtcgccatcggctatcagaacaacccggcgctaagtgcgacggtttttgtccctgatccttttattcccggcggtatgatgtacaaaactggcgattatgcccggttgctggatgatggctgtgttgactgttttgggcgtaaagacggtcaaattaagatcaatggacaacgaattgaaaccggagagatcgagcagcgcctgctggagtgctccggcattatcgaggcggtagtggttccttaccgcgtacgtgaaacgctgcatattgcggcagtggtctgcgtcaatgatagctatgatgaggtggaagttcgagggcaattggctgacagattgccgccattcgctattccggaatccttggtggtggtgacggagattgcaaaaagccatagtggcaaggccgacttggcgcagttgcggtatctcctgcccgcaactcagtgcaacgccgtgtccaccacgatatcagaggtgcatagtgacatggaacatgcgctgcatgctatctggcaacgcgtacttgatagacaagacattgatagcaatgcctcctttttcgccctcggtggcacctcattggataccatcagggttaaaggggatattaagcggcaacttggcttggagattgatattaccgatctctttaagtacccaacgctcacggcgttagcacattttctcgatactgccgtatcgccggaggatgcaattccaacgcgtgctgttgtctacagcgacatgccggtggcgattgtcggtatggcgggacgtttccccggtgcggcgaatattgcagcgttgtggacgctggttgtaggaggggaatcgggattaacactgttcagtgatgaagagttgcgcgcgcatggtgtgacacctgacacgcttaaacaagcgaattatataaaaaccaaagggattgttgatgatcacgaatggtttgatgcggatttctttggttatactcccaacgaggcggaatgtatggatccgcaaattcgcttattgcatcagtgctgctggcaaactctggaacacgctgggtgcgatcctgccacctttactggtgcgattggcatttatgccggactgctgacatccccccactggcttaatgcggtaatgcaagacactaccgactctaccgccctgtacaaggccagtatcctgaatatccattccgtcacagcattgattgcccatgcgcttaacctcaccggccctgccgtgacgcttgacaccgcctgctccacttcggcagtggctatccaccaggcctgcatcgcactacgtaaccgggattgcgatgcggcattggcgggcggcgtttctatcgagatgcctgcgtaccggggctatgaatatcatgaaggcatgatcaatgcgcgagatggtgtctgtcgtcctttcgatagccaagcctctggcacagtcaccggtgatggcttaggaatgctgctgctaaaacgtctggacgacgcgctggcggatcgggactgcatctatggtgtgatcaaaggttcggcggtcaataatgatggcaataacaaaatcggctataccgcgccgagtgtgatcgggcaatcaacggtaatccgcaccagtttgcgccgtgccggttttgatagcgacagtatcggattggtggaagcgcacggcaccggtacggtgctgggtgatcctattgagttacgtgcgcttaatgaggtgtttggcccaacacctgttccgttttgtgtggtttcagcgctgaaaagtaatattggccacctcaactccgctgcgggagtggcgggcgtcatcaaaacaacgctcgccctgcatcatcaagtgttgccaccaacagcgcactttcgccaactcaatcctgctatagatctatcgcgttctgcattgtacgttaaccagcaagtccaaccgtggccgtcgacgcgtcctcgtcgtgcactggtgagctccttcggcatcggcggcaccaatgccagcatcgcactagaagcacatcaacatgaggacgacccttcagcgacgggggtacgcgacagctatctgttgttgttctccgctaaaacacccgctgcgttagagttgcgcgtggcctccacactggaatatgtcaagcatggagtaggggtgcgcctgccggatgtggcttatacattgcaaactggacgcacagcctttgaccatcggcgggcttatttggtgagtcgtgggtcgaaaatcgatctctcctgtgccacgatattgcaagcggaaatcttcaatggtcagcgcacgacagcggagatctgcttcatgtttcctggtcaaggtagccaatatcacggcatggccagcgcgctctatgctcatcaacccatgtttcgccagcacatggatcgctgctttgctgcattccaacgctattcgacggtcgatctcaaggcgttgttgtttgacgatgaggatacgcgggatattgatcaaacgcagttcacacaaccggcgttgttctgtgtcgaatacagcctagcgcgcaccttgattgatctggggattacgccggacagtatgatcgggcacagtctgggcgagtatgttgcggcctgtattgctggcgtatttactcttgaggatgcgctgcacgtcattgaggcgcgcggacgtttgatgcagtccatgcgtcccggtagcatgatggcggtctaccttagtcgcgaacagttgaccccatggctagctgcagaacggggtattgaactggcagctaataatagcgcgcatttttgcgtagtcgcgggcgagcaggcggccatttcccgtttgagcacacgcttagtcgagggcgggatacagcacaggcgcctgaaaacctctcacgcattccactcggccatgatgacgccgatgctgcacgattttgcacagttgctggggcaaatcccgatgcacgcgccgcacaagcgctttatatctaatgtaagcggtacatggattactgaggagcaagctacctcgccggattactgggtgcagcaagtgcgcaacgcggtgctgtttagcgaaggtgcggcgcaactgttggtacaacccacgctgtttatcgaatgtgggccgggtaatacgctctctacctttattcaaggacataaccaatacagcgatcagccgaccctgttgacgctacgcaaagccaacgcggcgatcgatgatgagcacatgttgcatcgtacgctggcggcgctgtgggtcaggggggagaatattgattggagacgctttaaccagacggcactcggcaagcacattccattgccggattaccccttcgaacagacttattactatcgctatggtgctgcactttccggttatcgccagtatccaaatcctctgcgccgtccgcaagatgagtggctccagcgtgtgctgtggcggatgcacgacacatccttgcgggaggcgttctatgcgccgggcgaattgatcatcattattagtgctgacggcgacaagttacagcagacgctgatgagtagtggtgtcgacagcatcacaatgccgctgccgatatcgtcagaggacgacgtgtgggataatgaccgtattctgacgcatttccacgacatctgcgcacttttagcacacaaaacctaccgacagttacattgcttgtatgctcccggtgcggaggcaggatcatcgttgacacagtcgctctcgggactttatcgtgttgctcgctggtgtatgcacagcaccacgccgctggcgtccttaacagtattgacccatggtgcgtttcgcgtacaggaagaggataatcccgaaccgacgctggcagcgttgtcgggcgcagtaaacgtgtttgcccaagagctgcacccgaccgaagtacgattgatcgatatcgatgcgcagagcagtgatgagaatctgaacttgctaacccagcgtctggccccgaaacaagaaacggtaatggcgctcaggcaggggatgctttacctgcgacgctttatcccgacacggctgttggcgcacttaccccctcaaacagggtgtataccgggcaacgtactgtggattatcggcggtgaaaaggggattggccgcatgatcggcgaagcgcttgctcagcgtgagggagtccgtgtggtactgagcagccgcacaggttatcaccatgaagcggtgcagcaggacgcattagacgttatccactgcgacgtgacgcaggcggaggcggttagagcttgtctggcaactctgctcgaacgctatggacggttggatggcgtgatttttgctgccgacgctaccaccacattgacactgcatcaattgagcgaatctgcgctacgcgacacgctaacggtgaaagaacggggtactgctaatgtgctgcatgcgttagcgcaacggaatctgctggatgagcgtctgctactgctgttctgtaactcgttggctgccgtgaatgcggagattggccagacaggctatgctaccgccagcgcctatttggatgcactggcacagcaactgcgtacccgctacaaggtgaatgcgctcagtatcgggttggatgcattgcgtgagcagggcatgttgttggatgctataaacggcagtgaatacgatgtcttgcgtggactgcgcccattgatgacgggaacgttgctacaggcttataaacaacaaggggctgacaccagctactacgcgcgattatcccccgagtccgattggctgctggatgagcaccggatatccggtatcgccacgctgccgggaaccggctatctggcgctggcgtatgaagctctgcgccattactttgtgcaagaccaaatctgcattgatgaattggtctttttggcaccgttgactgtgatggacaactgcagtgttgacgtttttgttgacatttcacctaacgggcaaggagttagtgtcgaggtgaaatcaatgacggagcgctttagtggcacgttaacaacccatgccagaggcagggcgacgcgtctgatggtagacgataatgttgtgtgcgatctcacggggctgatgcgcgagatgcacactatcactcctccaacaaaggaattatcgagcacgcacttccactatgggccgcgctggcacagcgtacaacaactgtatggcaataccgcccagactcaggttttcgcaacgctggccctgcccaccgttgccgctaatgatacgatcgcactgcaccctgcgctgttggacatcgccagcagtgttgtcgaacaactgcctggttttcatactgattcggtacctttcctttatcaggatttacgcctgtaccgcccgttgccgaacaccttacatgtggcgctgactgtcaatcggcacgatgaggagggtgacagctacgctttcacgctctacgacatggcaggcgagatggttgcccgctgtgcggcaatggtgaagcgcaaggtacagctccacatacaagatgtcgatgacgacacgcgactgcgcgtgcccagtgccgataactaccaactgcggctggccgctgagggggagggggcaggaaagctagcgttgtgccctacgccgcgcttagcgctgggggattcacaagtagagattgaggtactggccaccggactgaactttaaggatgtgctgttcaccacgggattgctccggcagcagccgggtgaggctccgctgcaattgggactggagtgcgccggacgcattactcgcgtgggtaaaaatgtcactgaatttgccccgggagaggatgttatggcggtgctaaacggtggttttgtccagtacgcacgggtagaaagcgattgcgtagtgagaaaaccagcccattgtcgcatcgaacaggcggctgcgctgcctatcgcatacctcaccgcctattacgcactggtggtgcgcgctaatttgcaacccggagaacgagtattgatccacagtgcggcggggggcgttggcttggctgcgctacatattgccaaacgctgcggagcacagattttcgccacagcaggtagcgagcagaaacgcgattacttgctttcgctaggcgtacatgccgtagctgattcacacgacgaacagttcgctgccactctgctgaccgcatcggacggacaggggatggatgtgatccttaactccctcacaggccgtctgcttgacgctagcctcgcgctgctggcaccgctgggccgttttcttgagctgggcagcaaggacattgtggaagacaaagcgctaccgatgcgtttcttcgcccaaggcggcacctttattccgattaattttcacgcggcgcatggtgcgtttagtcgctacctgcaacagattgtcgcttggatagatgataacacgctaccgctccttccatgcaaatccgtaccattgcccgaggttgcacgcgccttcgccaccctgaccacgccgcagcatattggcaaggtggtggtaacgcatcgcactgcggcaggcatggaccggctgaacgccatgatagcagaaaggcgcctcggcggctatgcgctcagcatgagcaatgccgaggtgatgcgccaattgtggccgatactgaacactcgcagtccgtgggcgcaactgctgctctcacctcgggcgatcgatagattagcgcggggcaaccgggtggatcgcggtgtaccgtctgccgctaacgatacgattactcagcagacagtgaaaaagcggccccgtccggaaattggcgtgccttacagccccgcgacacgtgaagtggaacgcgtgctctgccaaatcctggaagagtatctgggtctggatagagtaggcattgacgacaactatgccgaattaggggcaacctcactcgatatggtgcaactgagtgggcaaatggcgcgtcactatccgcaagtgagcgtggtgtcgctgtacaaccacgccaccgtgcgccagctggcgacgttttgccagcccccggagggcgagtcaaatgcgccatcaccacagcctgcagtacagacgaatacgcgcgcgaatcagatagcaaaacgtgctttgcaaattgcgaaaaatactgcgcgcagtcacacgtctttgcattaa

SEQ ID NO: 296 - DNA, 2601 bp, synthetic construct
  misc_feature 1..2601: note = Synthetic: clbC
  source 1..2601: mol_type = other DNA
atggaatacgcaagcgaaatgaacggcatggaaatcgccattattggtatggcggtccgtttcccgcagtcccggacgttacacgaattctggcataacatcgttcagggtaaagagtgcgtcaccttctttagcgaggaggagttgctggccgaaggcgtcgaacagagtactctggacaacccggcctatgtacgggccaagccctatatcgagggcatatgcgactttgatgctgcatttttcggttacagtcacaaagaggcgcagactctggatccgaaatcccgcgtattacatgaagttgcctaccatgcgctggaagatgcaggctatgcccaacgtaccagcgatctgatcaccggggtattcgtgggggcgtcagaagatgtggattggctacggcgttcgctgtcacagattggcggcgatgcgctgaatcgttttgagtctggcatctatggtcataaggatctgctcgcacatctcattgcctacagtctcaatctcaacggtccggtgtatagtctctacaccagttgttcgacttctctgagtgcaacgcatatcgcttgccgcagcttgttgtttggcgaatgtgatctggcgttggcgggtggaattactatcgatttaccgcagaagtcaggctacttctgtcaacagggcatgatccactccaccgatggccactgccgtccttttgacagtcaggcttctggcaccctgtttggcgatggcgcgggtgtggttgtgcttcggcgtttggaagatgctctggcagcgggcgatcgcatctatgcggtgatccggggtagcgcggtcaacaatgacggtaaacaaaaaatcggttttgtcgcgcctggtcatgagggacagaaggcggtcatttgtgcggcctgtcatctggcagaagtaagcccagaaagcatcggctacgttgaaacccacggtaccggcacccgtattggcgatcctatcgagttcgctgcgttgacggaggcgttcgatacttcacaccgccaatactgtgcactaggggctgtaaaggcgaatattggccacacccacgcggcggcgggcgtggctgggttaatcaagacggcgctggtacttcatcaccggaccattccgccgctcgccaactatcaaatgcctaactcgaagctggatctggcgcattcaccgttttatatcccgatacagccgcaggagtggcccgcatcgcggatgccgccccgcgctggcgtcagttcttttggcattggcggtactaatgtgcatatgattttggaagggctgaatcctgcggtgcgcgatgaccatgaccaagtgcgagcaccggtgtttatccctctctccgcgccgtctttcgagcagttggatgagctgacgcaacagcttaccccgttgctggctacccttgatgcgtcaacgctagcctacacacaacaagtggcgcgccccgtgtttgattgccgccgagtgatacaagtggaaaacgatggtacgcaagcgatgctggcatcgctggataacctaatgcccgacgctccttggggcctacactgtccagatctacgtactacgaacgattgtacttacgcacagtggctggcgcattcggcacattatcaacgcgaagcgactgctctgacggcattactcgacggcatgaatattccacccgcttattgccacgctgaaacgtgggcggcacaagcgaacagcagcctgctaatcagaggctgccagactatcgccgcgctaaaaacctggatgaacctgctaccgacattaacactgctgtcgggtgctggaacaggccttttgcctgccgctgcagccagtggcatgattgcgacgcaagacgtgttgcatttgctgtgggaaatggagcaaaaggcgcttcatctctggcttcctgagcgccatgaacctatccccggctacgtgctggcttggcaaggaaatcccatcaccgatgcacagcgtaacgacagagggttttggagtgaagcgctgttggcagatacccgagaactgggggagggcgttcacagtatcaactgggttaggctgccgccggaaataagagaagacgttgatgtattgcgctacgtggcgcaactgtggtgtgcaggtatcaatgtggattgggccgtgtggtacggcactccgctgccgcaacgcgggagcgcatcagcatatcccttcgcacacaaccactacccattgcctggacgggtaatgggtagtgtggagacccaacccgaagcaggacccgaaacgcaccacccttatcaggcacgtcccgtgttaagcgtacctttcgtagccgcacactcgcggggtatgcagtatatcacaggtctgatggaactgttgctggaaatatcgccggtcggggtggacgacgacttttttgaattaggcgggcattcgctgctggtgacgcaattgacctcccgtttagaacgcgatttcaacgtacatatcgatcttttgaccctgatggaaaaccccaatccgcgcaatatctacgcgcatatcgcggcgcaactggggggcgaagacaacctcgaaatagcctgtcagtaa

SEQ ID NO: 297 - DNA, 870 bp, synthetic construct
  misc_feature 1..870: note = Synthetic: clbD
  source 1..870: mol_type = other DNA
atgatgaacgtggcggtaataggtgcaggagtaatgggaactggcgtcgctcataacatggcgcaatacggcatatcaactaacgttgtggatatttctcaatctcagttggataaatgccgacagatgatcgaagcgaacttacgcttatataattttcacccgcagcataaaaaaaagacgcatagcacagcggagataatggaaaatatccgtttcacaactgagttggacgacattgtagaatgtgatttggtgattgagaatattactgaggatatagagaaaaaaaatgcactatacacacggatgaatacgatctgcggcgcgtcgacagtgtttggtgttaatacgtccgccatatctatcactgcgttgtcaaaattaatgcgacatccggagaatgtagtcggcgtccattttatgaacccggtaccgctgatgcacaccgtcgaattaatccgtggtgtacatactgcagagcgtacactgaacatatttcaccacttatttgctcagttgaataaaaccggcatcgtggtcaatgattctccgggctttgtcactaatcgtgccatgatgatttttgttaacgaagccatatttatggtgcaggagcagatcgcccgtgctgaggatatcgatacgttgtttaaaacctgcttcgggcacaagatggggccactacaaactgccgatttgattggcttggatacgattttgcaatcgttgcaggtgctatatgaaagttttaatgatgataagtatcgccccagctttttattaaagaaaatggtcgatgcagggtatttgggcgtgaaatcagggcaggggttttatcgctatcaacagacgtacgccgagcagtga

SEQ ID NO: 298 - DNA, 249 bp, synthetic construct
  misc_feature 1..249: note = Synthetic: clbE
  source 1..249: mol_type = other DNA
atgaaaaagcaagatatgaaagccgccattcgggaatttctttcacgctcattacgtgggcatacgttgaacgatgatgacgatattttttctctcgggcttgtccattcgttatttactgtgcaaatcatactgtttatagaaaaaaattttcaggttgagctggaagtgagtgagttgaaaacagaacagattgctaccgtcaataaaatagtggagctcattcagcgacaaacaggcctggagtaa

SEQ ID NO: 299 - DNA, 1131 bp, synthetic construct
  misc_feature 1..1131: note = Synthetic: clbF
  source 1..1131: mol_type = other DNA
atgtgcactgaaaattatgagctggctcagcaagaggcagtcctgtttgccaaacaacatctcgccctggctgcacagaatattgaacgtcagcagtttattgtgccagacattatttcttgcgtggcgcaggccgggtacttaggtgcgtcaatcccccaaaaatatggcggacgaggttacgattcttatcaactgtgcgctttgcatgaagtgatggctggggttcacggttcgttagaaaatctcataacagtgactggcatggtcagcacgctgctgcaacgcgtgggcagcgcagcgcaaaaagcccattatttgccaaagctggcaacgggggaattgatcggcgcgattgccctcacagagccgaatatcggcagtgacttagtgaacgtggaaacagaattacagcaggatggcgacggttggcggctgaacgggaaaaagaaatggatcacgctagggcagattgctgatttttttattgttttaatccactgtggtaatcaattagcgacagtgcttatcgatcgcaataccgacggctttactatcactccactcaacgacatgttggggttacgcggcaatatgttggcagagctgcattttaatgactgccgcctgaaggaggatgcattgctcggtccgctcacccctggggtaccgctggcggtgaattttgcgctcaatgaagggcggttcaccaccgcttgtggcagcctaggattatgtcgggccgctgtggatgtggcagcgcgctacatccgccagcgtaaacagttcaagcggcggttattttctcatggcattgttcagcacttgtttgccacaatgctgacccaaacgcgcagtgcgcaactaatgtgcttcagcgcggcggaataccgcgaaacgctgcacccggccatgataaaccaaatcctgatggcgaagtatgtcgcttccaaagctgcggtggacgtggccgggaaggcggtgcaactgctcggtgcgaatggttgccatgccgattatgccgtcgaacgttactaccgcgacgccaaaatcatggaaatcatcgaagggacatcgcaaattcatgaaatccaaattgcgatgaattacatgatggggagtgaagcatga

SEQ ID NO: 300 - DNA, 1269 bp, synthetic construct
  misc_feature 1..1269: note = Synthetic: clbG
  source 1..1269: mol_type = other DNA
atgacgaaggatgtcgcactgatgttccctggctccggttcgcaatatgtaggcatggcacggtggctgtatgagcgttatccgcaggtgcgtactctgtttgatgaagctagccagatcacggaacgggacatggcagcgctgtgcctgtcgggtactttggtacaacttgctgagccgacggcaatggcgctggctatctataccaccagcgtggcccactttgtcgcgtggcagcagtttttggcgcaaaccggtgtccatgtcaacctacgttatatgttgggtcatagtttgggcgaatatgctgcgctgacctgtagtggtgcgctgagcttttcccaagcactggcgctggtggcgatgcgtagccgtttagccagtgagatcgcccgcgaaatggacgcgagtaccactatcatcaagcaagggaatcaggcactggtggcagcggcgtgcgaggtggcggagcgtgaaacccgacagcaggtcggtattgcctgcttcaattcgccgcaacagtttatgttatccggacaaaactcggccattatcgccgccgagcaatacctgctggatcatgatcgccaagtcgaagtagtgccgctgattggtggtgttccttaccacagcccactattgaaaccttgcggacagcaattgcgcaaggcgctggatcgctgcgaatggcgacgtccgtgctgcccagtgatctccaatgtcaacgctcaaccctatcccgatacggtcacgccagtccagtggctagaacaacagctctctcagccagtgcagtggcagcgctctctcacctacctgacaggacacttgtcaccgatcgcgatagagatcggtccgcaaagtgtgctgaaaaacctgctgctggagaatcgctatccagcaccagtttacgcatttgacaatcgtcatgatcgtgcgcaactagcattggtgctgggggataacatggcggtgaagaccgatccggaagccgtgcgccgccaacgcatcacgctgctcactaacgccctgacagccacacgccatcaccgcgccgccgatgtggctgccagtgcgcaattgaaagaattgctcagtcgtttttttgagcgcatccagcagattgagcagcgcggcacatccagcgaagaggacattgcttttttgcatgagcttcttgaacagggatttcaactcaaaggcagtagtcaggcagaaatcgacgcctgccacgcgcggttggcttccgacaacggcggacaggcgtaa

SEQ ID NO: 301 - DNA, 4797 bp, synthetic construct
  misc_feature 1..4797: note = Synthetic: clbH
  source 1..4797: mol_type = other DNA
atggaacagcaagggattatgagacagttgcctaccgacgaccaaacgatagtcgactatttgtatcgtatcgccggagaatatggggaaaaagccgctgtattgatgggggacgcggcgctgagctatcacgatcttaatgcacgctctaaccaactggcgcattatctgcgtgggctggggatcggcgaggatcgtgtggtagctatccgcctgccgcgcggcatggcaatgctgattgccattttcgctattgtaaaagctggtggtgcctatttaccgctggcgtacaatgcaccgcgcagccgcattgaaaatatactgagcaacagcggcgctgtttgtctgatcggtactgacgatggtgatcgctggccgattcctcgcgtcgaaatcgacagcgcggcggtcagtgccatgccaacgacggatctgcgttaccgaccacatgcgcggcaactggcgtatattatttacacctccggttccaccggtgtccctaaaggggtggcgacggaacatgcagcgctgctaaaccgaattgtctggatgcagaacgcttatcctatcagttctcaggacgtgctgtttcagaagacggtgtacacctttgacgtctctgtctgggagatgttctggtgggcgatgtacggcgcatctgtagtgctgttaccgtccggactggagagcgatccgcgaaccttggctcgactgattcagcgtcaccgcgtgtcggttgtgcactttgtcccttcgatgctgaacctgtttgtcgagtatctggagatgaaacaggatcctcgtttgaccgcctcattgcgattagtgttttccagcggtgagaaactcacggtccacagtgtggctcgcttttatcaatcggtggcgcagggtgatcttataaatctctatggcccgaccgaagcagcaatcgatgtcagtcatcaccgctgcctgcgcgggtacgactacgacgatatccctatcggtcaagcgattgacggttgccgactctatgtgctggatgaccatggtaatccggtagcagacggcgaagagggcgaactgtatctcgctggtatcgggctggcacgtggctatctcaacaacgtggcgttaactgatcgctgttttactatacatccaactttgcgccatttagggaaaccggagcggctgtataaaactggagatctggtgtggcgcgacggggaaagccaacaaattcattacattggccgtaatgattttcagattaaaattagagggttgcgcgttgaattgggagaaatcgaagcccatgcgatgcgtttcccgggggtacagcaggcagtcgtggtggcggatcaggatgatcccgacaatcagttgatttacgcttttgtcgtcagtagcgtgccgctcaatttggcggccttaatggacgcactgtccaaaaacttgcctgcctacatgctgccgaaccgtttgttggcaatgtcagagttaccactctccgacaatggcaagtgctgtcgtaaaacgttgctcgacttggcgcgggcgtactcagccagtcgcgtcgatttacgtgaaactcccgccgtgcgctacctaccgttgtcatcggctcaatcgtcgatgtggtttatgcaacaattggcgccgcatactgcactatacaataaccccaccgccttgctgcttgaaggagaactggatcgcacgcggatggacggcgcgattcgtcaattgatgagtcggcatactctgttacgtgctatggcggaaacccacaatggacaaccagtattggcggtgcctcagtgcgtatcgtcgcaggcgttgctgaccatagtgccactcccctcggtgagtgatgataacgcgctacaggcgatgatcaaccagcgtgcggcacaccccatgcccttaacgtcaggcacaccgctgtgccggtttgaactgttgacgctcgatgacgatcgtagcgtattgttgattcatctgcaccacatcatcagtgatggctggtcgaaaggcgttttgttacgcgaattgcaggccgcatataacggtgaatcgctgacgccagaaccgttgttggagtatgccgactacatggagtaccaagaagagtggcgtcagagtgacgcctatcaggacgcgatgcgttactggcaaaataccctggcggggacgttgccgattcttgatatccccaccgatcaaccgcgtcagaaggtggcacgttaccaaggcgcgtttgttgccttcgcattgtctgccaacacatgcgagcgagtgttggcggcggcgcgtgcgcagcgcgtgtcgttgtataactatctgctgaccgccttcgtcctcctgctgcatcgtaatgcgcgtcaacaggaatacatcgtcggtatgccgattgctgcgcggctgacgaaagaacaggagcatatgatcgcgccgctggtcaacgtactaccgctgcgcttacctcttgacgaagcggcatcgttttccgagttagtacagacgatcagaggtattctgtttgccgctttcaggcaccagcgtctcgaatttaccgacatagtgcgcgcagtgaatgtggatcgcagcgccggacatttcccaatatatcagtgcatgttccaactcgacaatatgcctctggcgagcccgacactaaacggtgtcaacgttacaccattattgttggataccagtgcatctcaggtggatatttcgctaagtatgcagcatatcgatgggcgcatcaccggcactttcgaatacgacgctgggttatacagtgcggatcgtattcagcacctggtggcgcagtggagagaactgctagatgaggccagcagccaacccacgcagttagtccgcgatttgattcgtttcacccctcgagagcacgcttggctggcgcggcacaacgccactgaagttgctctgccgcccgtagataacctgctggcgctagtgttaccgcactgccagcagcgccctacacaggtggctttgcgccacgctgacgacgccatgacctatggcgaattgcaacaggccacgatgcagatgtgtacttggctgcgtgcgcaaggcgtaaaacgcggagaatctgtcgcactgcaactgcctttttgtttcgaattgattatcgcacaattggcgatcctctctttgggggcgagctatgtgccgctggatggtaacgcgccagcggcccgcaatgccctgatcttggcgcaggcaacgccgtgcatgctgctggtggcgcaaccgctggaatcccctcatgggctgacgataccttgggtgctagtgcctgactggcgtagcctgctgacagaaataccgaacctgccagttagtgttgcgccagatgctttagattgcgatgcggtggtgatctttacctccggtactaccgggcagcccaaaggcgtccgactcagccaacgtaatctggtcaacttaaccgcatcctttatatccagctatcaagtgacgcatcaggatgttttgctacccattacctcagtcgcgtctgctagctttgtcggggaggtactgccgctgctcgccgctggcggtaccttagtattggcgcaaaaagcgcagagtttggatagtgatgcgctcattgcgctgctggcatctcagcgggtgactatcctgagcaccaccccatcgctttctgccagcctctctgtgctggctcagtcgatgggatcgctgcgcttgtttctgtgcggtggtgaagcgttagagtatgagcagatagcgccgcttctgccgcacatggcagtggttaacggttatggcctgaccgaaagcggtatctgctcgacctactttcctgtcgcaaagcgtagggagcaagaaacgggagcattgcccattggccgcccgattcagaacacccaagcttatgtggtggatgcatataatcgtttggtaccgccaggtgcctgtggagaactctgtttctctggtctgggcatttcgcccggctaccttgatgcacgacaggatcccgagcgctttgtcgagttgccggaataccctggcgttcgggtgctgaaaactggcgatcgtgctcgttgggcaaccgacgggatgttgttctatctcggcagacaagatcgccaagtgcagatccgtggataccgagttgaactgggcgacatcgaaagcctactgaaacagcatccggatattgctgatgcgtgggtcgatgtgcgacgcaatgcggcggcaacgccgctactagtggccttctattgcagcgtcaacggcgttgcgctggatgctcagcaattgcgcgtatggctcagcctgcggcttccgttgcatatgctgccgttgctctacgtgccgctgagcgccatgccgctaggtgtaaacggcaaaatcgatccccagtgcctgccgctggtcgatctgcggcagttggaagggccgggcgagtatgtccctccggcaaccgaactggaacagcgtctggcggagatctggcagcagttgcttggcctggagcgtgtcggcaccaccacaaatttcttcgatctcggtggacactcacttctgctagtacagatgcagcaatacatcgggcagcagtgcggtcagcacgtggcgttggttgacctgctgcgcttcactaccatcaaacgcttggcggaatttctgctggccccggacgcagcgcaagggaccacaggagatcagacacaactgcgtgcggcgaagcagcgtttggcctttggtcacacgcgctgggcagccacaaccgacagtcatcactga

SEQ ID NO: 302 - DNA, 3033 bp, synthetic construct
  misc_feature 1..3033: note = Synthetic: clbI
  source 1..3033: mol_type = other DNA
atggcagagaatgattttggtatagctatcattgggatggcggggcgtttccctcaagccgatacggtacaggcgttttgggaaaatcttctggcaagccgagagtgcatttccttttacagcgatgaggaactgctggcgatgggaatcagtcctgaatttgttcagcacccagattacgtcaaggccaaaggggaggtagctgacatcgataaatttgatgctgctttcttcggtatcgcaccccgagaggcggagctgatggatcctcagcaccgtgtcctgttggaaaccgcgtgggcagccttcgaagatgcgggctatgtggctgccgattatccgggggatgtgggtatttttgctggcaaaagcatggattcctatttgatgctgaacttgatgccgcactttaaacgcgttttctcttctggcagtttgcaggcggccattggtaacgacaaagacagtatcactactaccatcgcctatcacctcaacctgcgcggcccggcgatcacggtgcagacttcctcatcgacatccttggtggcggtgtgcgtggcatgccaaagtctgttgacttggcagtgtgacatggcgattgctggcggagtaacgctggggccaccagcaaaaaccggctacttgtcgcaggaaggtgggatcaccgccgccgatggccactgtcgggcgttttccgataacagcagcggatttgtgcccggcaccggcgcggggctggtggtgttaaagcgtgttgatgaagcgctgcgtgatggcgataacatctatgcggtcatcaaaggatttgccgtcaacaacgacggttcggagaagatcagttataccgcgccgagcgtggatgcccaagcgcgcgccattgctcaggcacagcggttggcaggtttgacaccgcaggatatcacttatgtggaagcccacggtaccggtacgcgtttgggcgatccggttgagttttctgcgctaagccaagcctttgcgggcgcgtcacaaaagcagtactgtgcgctggggtcggtaaaaaccaatatcggccatttggatacggcagcgggcgtcgcagggttgataaaaaccgcgctggcagtgcagcagggaataattcccgcaacattgcactttgaacggcccaatgcacagatcgatctgaccaatagtccgttttacatcaacactacctgtcaaccgtggcaaccggaaagcggcatccgtcgcgcgggcgtcacctcgcttggcatgggcggcaccaatgcccatgttgtgctggaacaagcgccagccgttgacttgcaagcgcgagcgcctgtacctgcctacagtattttgccgttttctgccaagaccgacagtgccttatcctcggggctggcgcgttttgccgattttctgcaacatgaatcgctgccggataggcgggatctggcgtggacactctcacagggacgtaaagccttcgcacatcgcgccgcgctcgtaaccagagatctacatgctgccgggacgctgttgcagcaggccgcgacagcgccgtttgctcgtggtgtagcgcagacacagctcgggctggggctgttgttttccgggcagggtagccaatatcagcgcatgggccatcagctctatcaggtttggccggcctacgccgatgccttcgaccgttgcgcgacgttactcgagcgtgaataccaactggatatccgccatgagttgttcagagcagaagtctcgttagcccaaggcgaacgccttgcgcaaacctgcctcacacaaccgctgctgttcagcgtcgagtatgccttagcccaattgtggctcagttggggaatcacgccaacggtaatgattggtcattcgctgggcgaatgggtggcagcgacgttggcaggcgtgttctctttagaggatgcgttgcgcttggtggcgcgtcgagcagagctgatgcatcaggcaccaagcggtgccatgttgatggtggcgctgcccgaagcacagattcgcgcactgattaccgccccgctggcgattgccgctgttaacgcccctgactattcggttatcgccgggcctacgtcggagatcctcgccgtcagccagcgtctgacggagcagaacatcatcaacaaacgattgcatacctctcatgccttccactctagcatgatgcaagatgcggcgcaggcgctacgtcaggcatttgagaatgtacgactgaacccgccgacgctcaccatcatctctaccgtaaccggcgcgcacgtcagtgccgacactctcaccacaccggactactggattgaacagatgctgatgcctgtgcagttctccgctgcactgcaagaggcgcaagctacatttgatgtcgattttctggaaatcgggccaggtgccaccctgacccagctgaccaacggtcatgcattaggtgatcgtctcgcgttcagctcgctgccagcaggtgcccgcagcagcgatgagcacaaacatatcttagataccgtggcagcgctttgggtgcgagggcataacatcgatttgtctgcgtttgcaggtgagcaaccgcgccgtgtctctctaccgacctacgcctttgacaaaatccgttactgggtcgacagtccagaagaacagaggagcgccgtaacgccggtggcggacgcgggaagtgtcatccctagcgaaccgtcggtgcgccgtcagccgcgtccggcgttttcggtgccttacgcggctccagagagcaaaacgcagcgcggcttagtggcgatttgtgaagcattgctgggaattgacggcttgggcattgacgacaacttcttcgaagctggcggacattcattgatgctgggcatgttgctggcgcaggtacaggaacggttcgccgtcactctttcctttttcgacgtgatggaggatgccagcgtgcgcgcgttagcgcagttggtcgagcaggagcagcaggatgatggagggtccgcacttgccgtgctagttaacgacatgattaatgagtga

SEQ ID NO: 303 - DNA, 6501 bp, synthetic construct
  misc_feature 1..6501: note = Synthetic: clbJ
  source 1..6501: mol_type = other DNA
atgacgatacatcatgccgcattggcgcgaatgttaccggcggaaaaaaaggaaaagctattacgacagttggcgcaatcaggcgtgtcgcccagccgcattcccatcatcaaagcggatccggcgcaggcaatcccgctgtcgtttaatcaggagcggttgtggtttttgcaaaaatacgacagtaccgccactaactacaacctttatgtggtgtatcgtttgcatggcgtggtggatatgccgatgctcacggaggcattgcgccatgtgcaagcacgccacgccatcttgcgtacccgtatcatagtacgtaacgatcggccttgccaggtgatagatgatgcgtcttcgttagttctcgatacggtcacgctggcagcgcaggccccaacttcagcgctagatgcggtaattcagcaggtgataaacacccggttcgatctggcacgcggtcctctgtggggggtaactcagattattcaacccgatcaaggttgtcatttggtgttttgcgcgcaccatatcatcatcgacggtatctcgttacggctattgttcgatgagttacagcagcagtatgctcgattgcatgctggtaatgaaacgtcgctgcctccgccgccgctgcaatatgccgattatgccttctggcagcgcgaatggttccaggataccttgctggcgaacgaacttgcctactggcgtgcgcgtttgcaagacgcgcctctgctgtcgacatttccatcgctgcacccgcgcccggcacaaccctctacacatggttcccggttcagcatcaccctggatgaaacattaagcctcgcgttgaaacacgtagcccgcacgcaggaaacaacgccgttcgtgctgatgctgactgccttccaactggtgctgatgcgttacgctcagcaacagcgattggtgataggcatgccagtatcggggcgcattcgcccggaattgcagagtagcattggttattatgccagcactgcggttatctacaccgattttaatggggttgaggtagggcgcgaggcgctccagcgtgtgaaggccagcgtgaaagagacgcaggggcgccagcaactgccgtttgaaaacttggtgaatatgctggatctccctcgcagcctttcccattcaccgttgttccagatcctctatatctaccataaccacgtgaccccacgcgcttttaccttggctggtgcgtattgggagcaggtgacgtatcacaatcagaccgtcaaatacgacatgacagtcgaggtgttccaaaacgacgccacgttcgacgtctcctttgagtacgatttggggttatacgatgctgatgtggtgaagcagattgccgaggcgctgcgtcagcactgcttatcgttgacatcatcactggagaccccgataggggcgatccctctgcatgcaccggagaccgcaacgccgcggcgtgatccgctcaacgccactaacgtcccgtggctcgggccgcaggatgtgctgcgtatcattgaacagcgctgtgtgcagcacccaaagcaactggcaatacagcagcatgacggcacactgacctacgctgagctctgggcgcgtgtgcagttcatcgcgatgcgttttcgagcgcatggcatacaaccgggcgatcgtattggcgtgctgttgccacgtcacagggatgtgattgcaaccatgttggcgacgtggtttgtgggggcgtgctacgtgccgttcgatattcatcagcctgccgcgcgtttgcaacgccttatgcaacgcgcgcgtttggtctgtctggtggtccgtcagccgggagagtggggtgaaattgtacaactgtcgttgccggaattgatgcaggacatgtcgaatgcgatccggtattctacaccttgcgcgctgttgccggatatgcaggcctacctgttgtttacctccggcagtaccggtgaacctaaaggcgtgtgcgttgttcaccgcgggttgctgaacttgttgttggatatgcagcgtacctttgcggttggctcgcaagaccggctgctctcggtgacgacgccaacatttgatatctcattcttggagtatctgttgccgctgatctccggtgcgagtctctatctgacagaggcggaacgcgccgcagacagcttccgtatgattccgctgattgccgactatcgaccaacgctgatgcaggcgacgccctcgttctggcacgggctgttgatggcgggttggcgtggcgacccggaactatgtgtgctggcaggcggtgaagcgctgccaacgaaagtggcggaagaactgttgcgctgttgtggttcattgtggaacctctacggtcccaccgaaacaaccatttggtcgctgaaatcgcagataacccaagcggaaaacatcaccctcggcgctcccattgccaatacccgtatatacattctggacaatgagggccatccagtgccgcaaggcgttgacggcgagctttacatcgccggggatggtgtggcgcaagggtatgatgggcagcctgagttgaacgcacagttcttcttgtcagaaccaggggtccccggtggcaggatgttccgcactggcgatctggtcaggagtgatgcgcaggggcagttgttttttgttgggcgcaaagatagccagatcaaactgcgcggttatcgtattgagttaggcgaaattgaacggacgttggcacggcatccgcatgtggacgccgcggtggtggcctgtattgaaagagcaccgttacacaaagcactggcagcattcatcatcaccagtgagcctccctcgctattcgagcaactgaaaaacgagcttcggcagcaactgccagactatatggtgccaacgctgtggcagagggtggccgacttcccgaacactgacaacggtaaaatcgatcgtaagcggttagcggaaaatttcgttgccgatagctcccttgtgtcgccgcagacgcaagcgctgagcgacacggaacagatgttgctggcgctgtggatgcgctatttgccgataaaaaacgtcgatcctgagtgcgatttctttcgcttggggggacattcgctgttggcggtaacgttggtagcagagatcaaccgcacttttcactgcgccttgaccctgaaggacattttccactactccacactacgggcactgagtgcgcgtattgcacagcaatctatcacggacgctgccgcgtctcaggatgactgggtgatagtgcacgaccctgagcatcgtcatcaaccgttcccattgacggatgttcagcgcgcctactggcttggacgacagacgggtgctacctcgatcgcgacccacatctaccatgaatttgacgtagaacactttaatgttacgcgttttacccatgcggtgaatgcgctgatcgctcgccatgaaatgctacgtgcgcgggtactccccgacggtactcagcagattctggcgcaagtgccggcgtatcagttagagcagcgcgatctgagtgctttgtcccctaacgcacgaaacgatgccttgatggcgatccgcgatcggctgtcgcatcatgtgcatcccgcagatcgttggccgctgtttgatttcagttattcggcttgcacggcgcaacatggccgcttgcatttcagtctcgatctgctgattgccgatgctctgagtatgcgcacgctacagcaggagttgatgatgctgtaccgtgagccccatgtgtcactgccgttgctaccgttctcttttcgtgactacgtgcaggcgctgttggtagagcaggcgagtgaagcctatgcacgcgatcaggcctattggcaacgggcgctgccgcagctgtatggcccaccaacgctgcccgtacagggcgatttggcgcaactgtctgcgatcaggttcgtacgtcgccgtcatcggctgtcagcccacaactggggagtgctgagcgcgctggcccaacgcacacgtatcaccaagacggcattgttgttgacagtctttagccaagtgctggcacgttggagccttagcccgacgtttacgctcaatctgacgttgttcaaccgcccgcagggttaccccaacgcagaggcagttattggtgattttaccgctgtcagcttgctgaatgtttgttacgacagccagcactcttatgcccacaacgctcagcgtattcaggtgcaactgtgggaagatctcgaacatcgtcgtttcagtgggatccgcgccagcgaggcgctgatccatagcggtcgtttccatgcgccgatgccggtggtattcactagtatgttggatatcgacggggagacgactgcgcaagaccctcgggacacaacccgttttactctgtgtccggacgccaatattacccaaacaccgcaggtgtggctcgatcaccaggtgatcgagttggctggggagttgcatttcaactgggacgcggtcgagcaactgtttgataccacgctgctggatcagatgtttggtgcttattgtcatgcgctgcaggcgctggttgccatgccgcaaagttggtggggggtaaatagttctctggcgctgcccaccgttagtgcaccggtcacgcaggctcctgcacctacggccttgttgcaccatggattactgcgtcaggcagcactgacgccacaggaaactgcgctgatcagtcctatccgtgaattgacctatcgccaactgtcgacggcggcggatcatgtggcccgcgccctgttagcgctgggcgtgcagcatggcgaccgcgtggcggtggtgatggaaaaaggctggcagcagattgccgccgtacacggcattttacgactgggtgcggtctatctgccagtggatccggtgctaccgccacagcgtcgccagcttttgctgacggtgggcgaggtgcgggtacaagtaacgcagccgggtctcacgcaattggagccgtcgctgcccgtgctgatcatcgacgacggaatgctggacacgcctgctgcgccgttgcctgaagtggctggggatgtcacggatctggcctatatcattttcacttccggctccaccggtaccccgaaaggagtgatgatcgaccaccgtgcggccatgaacacgctggaagacatcaacgaacgctttggcctcaatgcgcaggatagggtgttcgggctgtcatcattgagctttgacctgtcggtttacgatgcctttgcgccttttatggtgggtgcagcgctggtactgccggaagcaggacgggaaaaagatccgcgtcattggcagacagttatggcacacggtcatgtaagcgtctggaatgcagtgcccgcactgatgcagatgctgtgcgaataccacagcggcgatcggatgagttatccgacgttgcgtctggcactgttgagcggcgactggatcccgctaacgttaccggagcagatgcgcgagcggctcaatgaaacgatggacatcatcagtctgggtggagcgaccgagtgcgccatctggtcggtctactacccgataggtgaggtggaatcgacgtggaccagtattccctacggtcggggcctgcgcaaccagccagtatacgtgctaaatgcgcaactggaggaatgtccggtcggggtggaaggagagatttgcattggcgggatggggctggcacaaggctacctgaacgacgcagagaaaacggcggcgagctttgtctggcgcgaagcgagtggtgagcgaatttaccgcactggggatcgcgggcgctactttgctgacgggcaagtcgcctttttggggcgcaacgatacccaagtgaaggtgaatggttaccgtatcgaactgggggaagtcaaaagccaccttgaacagctcgacagcgtagggagtgccgccgtggtgtgccaccagggacagctgtatgcctttatcactgccgcagaaaacctgcatcctgacgatactgacgccctgttagcgcgtgttcgtgctcagttagccgtgcagttgccgtattacctgctgccccaacatttctttcttctcaaggtgctgccgatgacaggcaacggcaaaattgatcaggcggcaatggttcaagaggttatccaacgtatgtcgcaatctacatcacagaagtcgagggccctcgcgcacgcctcgccctatgaacagcaggtggccgctctctggtgcgaggtactacaacgagaacagatcggactgaatgacaacttttttgaagcggggggcggctcaatccagatcgtgctgttgcatcgccgtattgaggagatttttaaggttacggtacctattgctgagctatttcgcttaaccaccgtgaaaagaattgccggttatttgcaggctatgcaggacaatgcacgggcggtgaatcaaacacagcagcgtgatgcttcccgatcccgcgcccagcaacgacttgtacgtcgtcaccagcgtcagcgttaa

SEQ ID NO: 304 - DNA, 6464 bp, synthetic construct
  misc_feature 1..6464: note = Synthetic: clbK
  source 1..6464: mol_type = other DNA
atgacttacagtgaaagcgatattgccattgttggcatgaactgccggtaccccggcgtgcacagcgttgcggcattcgaaacggtgcttcgcaccggatgcaacattttggaccccaaagtgaccccgagcaacggccacaatcacattacgctgaacaacgtctatgagcacatggcggagttcgatgccaacttttttggctacagccgtgcagaagcagaaatcatggatccacagcagcgcgtatttctgacctgtgcttgggagatgtttgaacaaagcggttacaacccgaagcagcacgatgcgcgcgtcggcctatatgcgggcgtgagtaccagcttctatttgcttactcacctgatgaacaacccagacaagcttgcacaactcggtggtctccaaatcatggtcggcaatgacaaagatcatttgacgtcccaactggcttaccgcctgaatattaccggcccttgcgtgacggttcaggccagttgtgccacctcgctggtggcagtgcatctggcctgcgaagggctgctgagcggtcagtgcgatatggcgctagcgggtggcgtcacgtttcgcatggaggagcagcgcagctatgaatcgcacggtgatgggctgcaagcagaggatggcctgattcatacttttgatgcgcaggcgagcggtacggtctacagcagtggtttgggcatggtgctgctcaaacgggctacagacgcgcaggtgcaaggggataacatccttgccgtcatcaagggaagcgccatcaacaatgatggcggtgcgcgcagtggttataccgtgccgggtgttgatggtcaggaagctgtgatgattgaggcgcatagcttggcggaggtgacgccgcagcaaattcagtatctagaactgcatggcagcggcacgccgttgggtgacgctatcgaattcgcggccatcaaacgtgtgtttgggacgccagcgccgaatgcgacaccgtggcgtctgggggctgtgaaacctaacgtgggtcacgtagaaatggcatctggcatcaccagtctcataaaaacggtgctgagtcttactaaccgggtgttttaccctacgctgaattttcaacgggctaatccgcaactggggttggaggacagcccgtttgaagtggtgtcccgtttaacgccttggccggagggcaccacgccacgcaccgctggtgtgagtgcgttcggattgggcggcaccaacgcacatctggtggtacaagcgccgttatcgacgccgcaggcgagggcgcagcagatggggccttgtgtcgttgtactctcggcaaaaaaccacaatgccctggaacagatgcaaaacgccctgctggcgaaactggccgcacatccagagatacgcttacaggacgtggcttacaccctgcgtcacgggcgtttctccgccccggtacgtaagtgtgtcatcgcagagaattgcacccagctagcccgacaactccgcgacgcaccgatggtggaggcaaccacgggttgcacgatctactggagattggggcaccgtttcgttgttgcactcgagacgcttagtgactggctggcgtgctctgaggtgttgtcacaggcggtgggacaactgctcgagcattttccgctcgaaccagcctgtctgcaagatctctcccctgcgcaacggacgtttatcagccagtatgcgctgatcgcgttgattgacgaacgggaaacgttgaatgtggtgctatgcggcgacggtgatggtggctatgctgccgccgtactacgaggagactgcacactagagcaagcatggcacaggttgaatgcgggccaaccgtttgatgatgtgccgacgaatcccctcttgcagcccgatgtctgctctttgatgctggacgacgcggcgagtgatgccaatcgcaccgcgctagaggcgttagggcagttatggttggctggcgtcagcctagattggcgctgggtggatgcggcggagcgtatgttgggcagtcaacgcatcgccctgccggggaccgtttttacaccgcagcgctattgggtcgaggccgtacggcctgccacgttctcccacgaatcgtcgaataatctcttatcccgcgcaacaaaatccgatattattgcagtggtgacggagatatgggaacgcacgcttggtgtcagcattgatgatcaccacgccagcttcttcgaactgggtgggcattcgctgttagcgtcaaccattttatatgatattcagcaacgttacggcatcacctgtacgctaagcgcattctttgccgatcccaccatcgagggattaagttgctatctgctcgaacaaggcggcagtgagacagcggtttcagcattgcctgatacggtctttgcccctgaccagcagcacctaccttttccgctgactgacgtgcaacaggcctattgggttgggcggcgaaaatccttgggattgggcaatatttccacccatatctatgtggaatatgaactacaggggctagatgaaacagcgttcaaccgcgcgctcaatgcggtgattgcgcggcacagcatgttgcgtgcaattgtcaacgatgacggtatgcagcagatattgccaaacgtgccggaataccacgttgccttctataccacccagtgcgaggatgcgtttcaacagcgttgccgtgagctgcgtgacaccctctcacatcagatgattgattgtagtcgctggcctctgtttcagatggaggtcgtggttgatccgcagcaaaaagcccggctacatgtatctatcgacctgctgatagccgatgcatggagtctggagttgttcattcgggaactagcttatcactaccgccatccgcaggcggcattgcctacattgacgtacagtttccgcgactatgtgctgaccttaaaatcctacgagaaaacgccgcagtttgaacgggcacgtgactactggcgcgcccgcatcgaaactctgccgccggggccacgattgccgctgcgtaccgaccccacgaagctggaaaaccccacattcgtgcgccgtagctattgtctatctcgtgctatctggcagcgcttgaaaacgcaggcgggtcagatgagtatcacgcctactacactgctgctcacggggtttgctcaggtgttggcgcgctttagttcctcgccgcactttagccttaacctgacgctgtttaatcgtctgccgttgcacgcagatatcaatcacttaattggcgattttactgcactgacgttgctggagattgatatgtcgcagggagaaaccttgcaggcgcgggcaaacgtgatccactctcaactgtggcgcgatttggataaccgcttgtttggcggtatccaggtctctcgactgctggtacaaactcaccgcgatccggcgaaatcggtgattcctatcgtgttcaccagcttgctcaatcaatatgaggcgagctgggaaacggatgatacgctgttcaaccaaccgcaggatgatctgtacagtatttcacagacaccgcaggtgtggctcgatcatcaggtaatggagcgtaacggggaattgcactttaattgggacgtggtggaacaattgtttgaaccggcgctaatggatcagatgttccagtgttattgccaactcctgcacgccctagcccagcgaccacagctctggcatgagactcaagatgtgctggcgctgcccaccgttagtgcaccggtcacgcaggctcctgcacctacggccttgttgcaccatgggttactgcgtcaggcagcactgacgccacaggaaactgcgctgatcagtcctatccgtgaattgacctatcgccaactgtcgacggcggcggatcatgtggcccgcgccctgttagcgctgggcgtgcagcatggcgaccgcgtggcggtggtgatggaaaaaggctggcagcagattgccgccgtacacggcattttacgactgggtgcggtctatctgccagtggatccggtgctaccgccacagcgtcgccagcttttgctgacggtgggcgaggtgcgggtacaagtaacgcagccgggtctcacgcaattggagccgtcgctgcccgtgctgatcatcgacgacggaatgctggacacgcctgctgcgccgttgcctgaagtggctggggatgtcacggatctggcctatatcattttcacttccggctccaccggtaccccgaaaggagtgatgatcgaccaccgtgcggccatgaacacactggaagacatcaacgaacgctttggcctcaatgcgcaggatagggtgttcgggctgtcatcattgagctttgacctgtcggtttacgatgcctttgcgccttttatggtgggtgcagcgctggtactgccggaagcaggacgggaaaaagatccgcgtcattggcagacagttatggcacacggtcatgtaagcgtctggaatgcagtgcccgcactgatgcagatgctgtgcgaataccacagcggcgatcggatgagttatccgacgttgcgtctggcactgttgagcggcgactggatcccgctaacgttaccggagcagatgcgcgagcggctcaatgaaacgatggacatcatcagtctgggtggagcgaccgagtgcgccatctggtcggtctactacccgataggtgaggtggaatcgacgtggaccagtattccctacggtcggggcctgcgcaaccagccagtatacgtgctaaatgcgcaactggaggaatgtccggtcggggtggaaggagagatttgcattggcgggatggggctggcacaaggctacctgaacgacgcagagaaaacggcggcgagctttgtctggcgcgaagcgagtggtgagcgaatttaccgcactggggatcgcgggcgctactttgctgacgggcaagtcgcctttttggggcgcaacgatacccaagtgaaggtgaatggttaccgtatcgaactgggggaaatcgagcgctgcattgcgcgacatcccgatgtggagcagtcagtggtggtggcagtgggtaattctcaacatcgtcggctggtcgcttttgccaaactgcacgatcgccaccaggcgcaggcattgcaagctaaggaagcggaggcggcggcactggcgcagggtattattgtgaatccggcacagcgtctagcgttcaaactcaaggagccacatattcgcgcgctggatggtctgggcattgcactgacggcaccggcggatagcacacgttacatcaaacgccgcagctatcgtcatttcagcgcgcaaaaaaccacgctggcacagttggggcaattgctgtcgggcttggggcagatgcgtctacccggtctaccttttgccaagtatgcctatgcgtccgccggggggctatacccggtgcaaacctacgtgtacctgcatccagacaagatcgaagagggagtatccggtatttactacttcgacccgcgacagagctgtcttatgccggtagcaccagaagtcgagctgaacagtggttttcatgccggacctaatcagtctattgccgatcgggcggcattcacgctgtttatggtggctgatatggcggtgatctcgccattctatgggcaagaggcagcttggcacttctcggtgatggaagcaggtactctctgccatttactggaagaagatgcgccgcgctacggattggggctgtgccaacttgggatggcagacttttccgctgtggcatcgcattttcaattgtcgccacatcatcgctatgtccattgcaccgtggggggcgcgatagggcaagaggcggcaagtgctgcagcattgctgcgcgatttctccacctatgagaaaccgaaggaaaccgctgcgccgctggacatgcagagctacaaagatgccatgctgcgcggcctgcgtcagcaactgcctgactatatggtgccgagtgatctgatgttagcgaccgattttccgttaaccgctaacggcaagctagatcggcaaaaattacagctgcagggcgaacaaattgcccaccagcgtgacggcgtgggtccaatccaggtggacagtgcgttacaacagcggctggtggcgctctggcaggaggtactcggcgtgagccacgtgtcggccgaagacgatttcttctcgctggggggcagttctatagaattggtgcgtattcagcaggcacttgaggcgattatcgggcaggagattcccattgtcgatctgttccgtctgccaaccatcgcggatgtggcgcgttaccttgatgagcaactgcacaatctacccgccgcccacgatatcgtattagcgcaggctgaggtttcgcaggtcagcgctgcgcgcgagaatctggcgttgcggcgtaaacgtgcccaacagggagaaaagggcgatgagtg

SEQ ID NO: 305 - DNA, 1464 bp, synthetic construct
  misc_feature 1..1464: note = Synthetic: clbL
  source 1..1464: mol_type = other DNA
atgagtgagcagagctatcgtagcgcagggacactgttggcacagttggcgtccggagaaaccacctcagtggcgttggtgaatcactatttttcacgtatggcgcagtttaacaagccgttgaatgcggtggtgcagcagcactatgcgttggcgttggaggccgccgcgcgcgccgatcgtgagcgactggaggggcgcgcaaggggcgtgttgcacggattaccttgtactgttaaggagtcgtttgacgtgcaagggtggttaaccacgtccggtgcccattatctgaaagataaccgggcgacgcaggatgctccttccatcgcacgcttacgcgctgccggggcgatcctgatgggaaaaaccaatgtgccgatgatgactgccgattggcaaacctataacgatctgtatggcaccacccataacctgtgggataggcaacgttcacccggcggatcgtccggaggagcggcggtggcggtggcggcggatttcacccccgtggaatttggcagcgatttgtttggctcactgcgcattcccgcacactacacaggtgtctatgcccatcgttgtagcttggggttgatgtcggtcagaggacatgtgcccggtggtggtccgcaagcgactgatgagccggatctctcgacggcgggccccatggcgcgcagcgcggcggatctgcgtctgatgatgcgcgcattgagcacattctgggtggaaccgccgcgtattcccgattttagccgttatcaggccaaagcaaactaccgcgtgtgcacgtggtttagtgcgccccaccatgagatagatcaacagatcgcacagcgttttcaatcgtttatcgacaagttacgcgcacagcctggcgtggaggttgatgatgctatgcccgccgatatcgatcccgacgcactgtttgatatagcagtcaaactcagtggtcggctggtaagtacggcgctcaatggacgccagcgtcttacagccgggctggctgcgctgggcttcaggcttgtgggcaagctggcggatgtgcctgaggggataacgagctattaccagggcatgctgaaagacagtggcgaacagcggaatacagacaaactccgccacgaatacagccgggtgatagaaacccttttcgcgcgttatgacgtgttgttgacacctgtcagcccggtgctggcgtttgcgcacatgcagcagccggtgcgtaagcgcaagcttatcgtcaatggcgaaccgcaagattacaacgaacatttgttctggaacatgttagcaacggtttttggtttgcctgccaccgtttacccattggcgaaaacgatggatgagcttccgtgcggcatacagatcatttccgggcattttcacgatgatgtgacgattaactttgctgagttctgcgaaagcatcagtggcggattcacggtaccggaagggtactag

SEQ ID NO: 306 - DNA, 1440 bp, synthetic construct
  misc_feature 1..1440: note = Synthetic: clbM
  source 1..1440: mol_type = other DNA
atgacggtgaataagcttgagccagacaacggcacgccgaacgatgagctgttcacggtggcgggtatgtttgacggctcgctgtataaattactcctgcgcatggcgttgccgatgtttgtcggtatgttaacgcaggtgacctatgccatcgccgatattttttggctgagccacattgatgtcaccaacagcggcatcattgccggtgtggggctggttttccccgttggaatggggttatttgccatcgccaacggcattcaaatcggtatgggatctttgctgtcccgcgccatcggcatgcagcggttggatcgtgcacagcggattttgtcggtcggtatcatcatcgcgctattctttgcgatcgtgatcaccgtacttggctatgtttatgcccagccgctattgcgctcactgggggccacgaagagcatcatcggctacgcaacggagttctattattattcgctgctgaccgtgttcagcatcatgttgattggcgtcatgatggggctgtttcaaggcgcgggtaagatcatggtcatcatgaaggcctcattgttgggggcgttggtaaatattatgctcgatccaatcatgatctttgtattcgattttggtgtgaaaggggtggcattggcgtcgtttctggcgcaactgagtatggtcgcctatttcatctacacattgatgggattacatatcgggttgagtatacgcatcgcgttacgcccattctcctggaaaatctaccgagagtttctctccgtcggcatggcgcagatgctgatgcaactgatcattgcggtcggcatagtgatttataatttttttatcgtcaggctggacgtaaacgcgatggcagcgtttacgctcactggtcgcatcgactatttcatcatcacgccgatgctggctatcgcgaccgcgttgctgactgtggtggggcaaaactggggacacggaaatgtcacccgcacgctgaatgcctattgggccgccgtcgcgctggccttctctattgtgctggtgttagccgttatgcatattgtactggcaccctggatgtatcccctgtttactcgtgttgttgccgtgagtgactatgcggtactgcaaacccgtatcatggcactggcactaccctttgtcgctatcagcctgttagcaagtgaatattatcaggccattggtaagccttggtattcggtgttgttgacgctgatgcgtcacgtgtttatatctgtccctgtcgtatacctgctggcgattgtgttagagatgcgtattaccggcgtctattttggggcgatgagcggcacttttgtggctgcgttgttggcgtggcgtctgctgcgtctttcacctcgtctgctgcgatggaatcaggaggcggtgcgctcccaacacttggatatggaggtggcaccatga

SEQ ID NO: 307 - DNA, 4368 bp, synthetic construct
  misc_feature 1..4368: note = Synthetic: clbN
  source 1..4368: mol_type = other DNA
atgatgtcgggcaatccgttgtcgtggccacaggaacagtgccacatcattgatcaactgtatccttacagtgccgtaaacatcattggtggtgtggtcaccattgagggaattgtcgatctgccccgactacacgcggccatccagtccgccattcggcaatttgatgcgttgcgtatgtggtttgtgatgggagaggagagtgaagtggtcagccaagtgcagccttatcactggcgcgatatccgccatttgactttttccccagattatgacaaggagaacctacgcccagcggcgatcgaaacatttgttgatgagtggttccggcagccctttaccctgctggcgcacgatttgttcgagtttgtcacctttacgtgcggtgaacaatacagcggttatttatttaaagcccatcacggcattgctgatggttggtcaatggcgttgttgtcaaaccatgtcaagcgagcatacgaacagcaggacgtaccagatgatgcgtcaccagcctatagcgcatttctggcgcagcagcaatcttatcaggcgtcaacgcgctttgccgttgatcgtggctggtggcgtgactatatcgacgaataccgcgactgctttccagacagttcccccatcgttactactgagggcatcagttgttccacttggcttgagccagccatgattaaccgtctctacaggttgtgtaatcgttatggctgtacgcttaatacgctgtttattgcactcttcgctctctatcgcgccagagtatggggggaggaaaaaggcgtaataggcgtgccgttagccaatcggcatactcgcgaggcgcggcgttgctttggcatgtttactaatcaattgccgctggcctatcgactggtgcgcacggaacgattctgtgaacgggtggcctttttccagcgcgaactgaaacgcggttttaaacacagcaaatatcccattaccttattcaaccaggatttggcggaacaggggggagggaagttgcgggcgttcgattattgcgttaattattataactttacctatgaacgccacattgcaggtgccgcgcaacgcgtggagagttactacagcggcgagcagtcctataagctacagattgttttgcaaactgttaataatcataaagaaagccttaggttgagtctggaggcgctgcgtagcgctttcactccgcaccaactgacagcgatgaaaaatgggctgttggatttagtcactgcattggatcggcaacctgacgctcgtctaggcgatctcgaagtctatcctgcgccgcacgtcgcattggcatgtggctcactcaaaccctcatttacttcccgatttgcagcgcaagttgtggagcatggtgatcgcacagcactaattgataatgaacagtctttgacttatcggcagttggatgacgctgttgagcgggttgcgcgttatctgcgccaacaggggatcggacgcggtcaagtggttgggattatcgctgagcatagcgctcaaacggtgatggttatttatggtattttgcgctgcggcgcggcatttctgccgctaaatcccgcgttgccaacgacccgcctttacgccatgtgtcgtaaagcgcaggtagcgcatatcctttacgatccggcgatgcatgaattgacgcaggcgctggcgtttcccgcgtccagcttgctccaggccttagccacttcggcgcttgctagagaaccttggccagctattgaaccgcaggatctggcatatgtgctgtttacctccggatcgaccggcgagcctaagggcgtgcaagtctctcatggcaatctggctaactatctccatttcgccgccgaacgttactttacagcgcaggaccgggctgcgctctactcttcgctgtccttcgatttgaccatcactacgctgttcgcccccttatgtgtgggcgctagcatcagcgtttgtcggcacgccgagagcgaaacattgctgcgcatggccgtggttgatcagcccaacacagtgatcaaactgacgcctgcacacctgcggttgttgtgtgcggctggcataagcagtgagcagattcgtaccttggtggttggcggggaggatttcaagcgggatctggcgcgtaaggcggctgctctctttcctcaggccgtgatctataacgaatatgggccgacagaggccaccgttggttgcatgatctatcgctacaccgggcaagaaacgctgccttcattgcccatcggtatggcgatcgacggttgccaggtagcaatttgctccccttggggctgtccggtaccggaaggcgaaactggtgaattggtgatttatggcgcgtcagtgacgcagggctatatcgatgcgccgcaacaaacggccgccgcctaccttaaagataccaatggggtgatgattggctatcgcagcggtgatattggctacgccatcgctcctaatacgctggtttatcagggacgtaaagacgatcaggtcaaaattaatggctatcgtattgagctgtgcgaaatcgaacaagcgttgttgagcgcacctcaggtagaaagtgcggcggtggcggtgattgatgatgtgcaggggcagcacagcgggttgctagcctgcgtgacaccgtcatctgttgatgtagctaccgtaatgcaacatctgcgtcagcaattacccacctacatgcaacccaagcagtgctgtgctatcgcccaactgccgctatcgcacaatggcaaggtggacgtgcgtcagatggtcgcaacggtccgaaacaccgcgcctgcatcgggtagcgagcggctgggagatgcggcgataaggcattcagttcgggtatgtgtggaaggggcgctggagcaaactgagtttgatgataacgaaaatctttatgtcttggggttggactccatcaagagtatccagattgcagcgcagctcaggcaccacggctggacgatgtcagcggtgcaggtgatggagtgtggaacagtgaacgccatctgtgagtttctcgctagtcatacaacggtgtcacagctcgcgcagtatgctcataacacccgtatcgatctacccgcattgcgttggtttacgcagttggcgttgccagtgcccaatgtttataaccatgtcattgtgctgaaagtgttgccgggctgcccgctcgagcagttacacaacaggctgcatacattaatccagcagcagccagctttgcacagtgcgctggacgctgaggggcggttgctagtatgcgaccctaatgtgtgttatcccaacgaggtgctcacggaatattcgacggcgcagtggacgttggcagaggtgatcgctcagtgcaatagcatgcttgacgtgacaaatggtcgagtctttacggcagctctgttacacgccccgcagcctgcgtctagcacgctggtactctgtgcccatcacttgtgcgtcgatatgcactcttggtatttaatcctcagcacgctggacgccgtcagcactgtcaatggcaccagcaacagcggactgcatcgctggaatgactatctggcgagcaaaacggtagattccgccacacacgaaagctggcgcacagtatgtcaaacgctaccgctgcatttcccacccgtgtcattgccagatgactcattacctcgtacgcgcgcttggcgtgaagactttcgccatccttgcgtcaggaggttgtttgaatccagtggtaataccgcgtacagcgccgaaacctatgtgctaacggcattggcgctggtgctgcgttactacagcgaagagccttggtgtcgcatcgagatggaaggcatgggacgcggctgctggcccgatgagcctgacgttgcggatacggtcgggtggtttactctattttatccttgggcgatccctctgcatggcgatatggcgacactgttatctgcgatcgcttccgatttggcaaaacgcacgcatggtgggggagattacggtctgctgcaaatgcggcatgcaccggaagactctcttgcgcaagggatccgaatgaattacatcggtgtgcaggcgcaaccttcactccgctattttcatattgatcactttaatagtgatatctataccgcgcctgaaaacgcgctcggctgcgtgctggagtttaatattgcgcgctcggcagctgatggtctctccttccactgccgtttcgatcccacgcgcattgcgctgaatgatgtgcaactcctgctggcgcgctacaagaacagcctcactgatctggatgcctggctgtgccaacacagcgccacgctgacgggcgcgccgactttgtggacactataa

SEQ ID NO: 308 - DNA, 2460 bp, synthetic construct
  misc_feature 1..2460: note = Synthetic: clbO
  source 1..2460: mol_type = other DNA
atggcaaaggatgattttacctgtggctcactggatattgccattattggcatgagcgggcgtttttccggtgcggaatcggtgcccgagtggtgggataagttgctggcgggtgaggagttcactcaaccgacatgcacagaagacgataatgggaacccttggattaggctgcgcaatataatcaccgctccctatgacttcgatgctgcatttttcaatattccgcctggcgaagccctgttgatggatccgcaacaacgaatatttcttgaatgctgctacaacgctcttgagcatgcgggctatattcccacgcaacttaagcgagtcggcgtctatggggcgacctacgccaataactattttatcgatcgcgtctatccatacctgaagatgagcggcgatcaccattatcttcaggcgcagattggtaacgaaaaggattacttgtgtgctcaggttgcctacaagttggggtttaccggacccgctgtgagtgtacagaccgcctgttccagttcgttggtggcagcatatttagcatgcgaggggttattgacttttcaagccgatgttgcgttggcaggtggggtcaccttgggttttttacaggcgcacggctacagtccgcaaggtgataagctggtatcgcaagacggacactgcgccccgtttagcgctgaggcgaccggcacggtatacagcagcggtgcgggagttgtggtgctaaaacgtcttgaggatgcactacgcgaccaagacagagtgtatgcggtgattaaaggaggtgcggtaaataacgacggcgggcgtcggctggggtttgtcgcccccagcgtagaaggacaggtagaagcgatcaatacggcgcttgccgccgcagaagtcgtaccgactgatatcgcactgatcgaaacgcacggtaccggtaccccgttgggtgatgagattgagcttgaagctctgcaccgggttttcgcaccggcatgtgcgccgcacagcattcaattgggtgcggtaaaagccaacttgggtcacctgggagtcgcttccggtatcgtcagcttaatgaaaaccgcactgacgctatatactggtttagttcccccgcagatcaacctagtaaataaacataaaaaactgttgcagccagcctcgccattttatctcagtgatgtagtgacttctgtcccacaaacgaaaaggatccatgccaccgttagttcgtttgggctgggagggaccaatgctcatttggttctgcaaaactggtgcgagacgcctgcgcaagcggtgcaagaaaatgagcgccgcctgtttttcttcagcgccaaaacgccattagcccttagacaacagttggatgctcattatcacgctctggcgacctatgcggaagcagataaagatcgtatcgcctatactctcgcgcaacgccgggcacatttcccgtatcgctgtgcgctggcggcagacagtgtggtagcgttgcgtgcaagtttggcgaaactgcgggatgctgacatgtcttttacccctatcaatatggaaactaccttggtgttcctatatccggaccgggacgataagctggagagtgccctgacgcatttgctggcttgtcaacctaacttgcgccagcggcaccagcgcctgtctcaggatgttgcacaaatctgcgaaccagccgattggacgccagcattgcgtcagtttatccagcaggtgagtcttagtgaatggctgatcgaacaaagtatctcgcctgtgcagcacatcggttacctgacgggggctgcagcagcgcagtatgttgcgcgcatcatctcactggaaaacgctgttcagcaggtgattgtggctgaaacgacaccggagcagacgctggccggcaacagtgaactgagcgaaatattggctaatctggcggtgacggagggaacgctgatgctggaaatcggtcgcgcggggacgttttccatattgtatcaccagcatgcgcagtgggttgggcaaacagtattttcccccatgctgaatacggatacgcctgaggacatcctgccgctgttgggcactctttggcaacgaggagtgactatttgtttaccggaaatgccagccgtgcagactataggcttacccggttattcgtttgatcgcgtccggtatgaaattcagtccagtgatgcgagagagaacgctatgttaccagtgagttatttgtcggtcagcgactttgtggagaaaacctggcgttcattgttatgcatcgatcattatgacgaacacgcggttattttcgaatacggagcgacctcgatgcacgttatctctttcgtcgacagctgtaaccacatttataaaatcggactgactgccgccgatatttatgccagacccgctatccgtgagcacagtgaatttatctccgaatgtgttgatggtattttatga

SEQ ID NO: 309 - DNA, 1515 bp, synthetic construct
  misc_feature 1..1515: note = Synthetic: clbP
  source 1..1515: mol_type = other DNA
atgacaataatggaacacgttagcattaaaacattatatcatctcctgtgctgtatgctgctctttatttccgctatgtgcgctttggcgcaagaacatgagcctatcggggcgcaagatgagcgcctgtcgacattaattcaccaacggatgcaggaggccaaggtcccagccctttccgtaagtgtgaccattaagggggtacgtcagcgatttgtctacggtgttgccgatgtggctagtcagaaagcgaatactctagacacagtttacgagctgggatcgatgagtaaggcgtttaccggacttgtggtgcaaatactgattcaggaaggcagactccggcaaggggatgatatcattacctatctgccggaaatgcgcttgaattatcagggaaaacctgcttccctgaccgtggctgatttcctttatcatacatcaggattgcctttttcaacactggctcggctggaaaaccctatgcctgggagcgctgtggcacagcaactgcgcaacgagaatctgctgtttgcgccgggtgcgaagtttagctatgcctccgccaattatgatgtgttgggcgcggtgattgaaaatgtgacgggaaaaacctttacagaggtcattgcggaacgactcacgcagccgctgggcatgtcggcgactgtggcagttaagggggatgagattattgtcaacaaggcaagcggctataaactgggattcggcaaacccgttctgtttcatgcgcctctggcccggaaccatgttcctgccgcctatatccatagcactctgcctgatatggaaatatggatagacgcctggttgcacagaaaggctttgccggcaacgctgcgtgaggcgatgagtaacagttggcgtggtaatagtgatgttccgcttgccgcagacaatcgtatcctctatgccagcggttggtttatcgaccagaatcaaggcccttacatcagtcacggtgggcagaatccaaacttttcttcttgcattgcgttgcgaccggatcagcagattggcattgttgcgctggcaaatatgaattcgaatctgatactacagctttgcgcggatatcgataattatctgcgcattggcaaatatgctgacggcgctggtgatgcaattacagccaccgatacccttttcgtctacctcacgttgttgctgtgtttttggggggcggtggttgtagtgcgcggtgctttccgtgtttatcgcgcaacggcgcatggccctggaaaacagcagaggttacgtttacgcgtacgtgactatatcatcgccttggcggttcctgggctcgtggccgccatgctctatgtcgcaccgggtatactatctccaggacttgactggcgttttatcttggtatggggtccatcgagcgtgttggcgataccgttcggaattatcctgttagctttcgttctgacattaaatcatcaaattaaacgaattctattacacaacaaggagtgggacgatgagtaa

SEQ ID NO: 310 - DNA, 723 bp, synthetic construct
  misc_feature 1..723: note = Synthetic: clbQ
  source 1..723: mol_type = other DNA
atgagtaatatcagtttgtattgtttgccatattcaggtggttctgccgccatgtattataaatggcgtagcgtgctgtcggacaatattactttgcggcctttagaacctgcggggaggggaactagaatacgccagccgctgtgtcttacgatggtggatgccgtcgctgacctttatcaacaatttgtgaaacactacacaggtggagactacgccatttttgggcatagtctcggagggatcatggccttcgaactggtgcattatattctcgatcatggacatgacatgccatgcgcgctgtttttttccggctgtcgcccacccgatcgggcctctcatgaagtaatactgcataccttgcccgatcaggcgtttatggaagagatcgtcaagctgggcggaactccggttgatgtctttcgtaataaagagttaatgacaattttcacccccatcattaaaaacgattatcggctctatgagcagtatgtatttcaggccaaggcgcgcacattaacctgtccgatcgtgctatttcatggcgatgctgacaatctggtaatgcaggatgaattacttgcatgggaaaaattcaccacacgaaagacgcggactattatatttcctgctgccgatcatttttttgtcgataagcattttgaacaggtggtaggttatgtgaaccagacgattgaatcactcgaaatagtagggtag

SEQ ID NO: 311 - DNA, 213 bp, synthetic construct
  misc_feature 1..213: note = Synthetic: clbR
  source 1..213: mol_type = other DNA
atggataagttcaaagaaaaaaacccgttatctctgcgtgaaagacaagtattgcgcatgctggcacaaggtgatgagtactctcaaatatcacataatcttaacatatcaataaacacagtaaagtttcatgtgaaaaacatcaaacataaaatacaagctcggaatacgaatcacgctatacacattgctaacaggaatgagattatctaa

SEQ ID NO: 312 - DNA, 513 bp, synthetic construct
  misc_feature 1..513: note = Synthetic: clbS
  source 1..513: mol_type = other DNA
atggctgttccatcatcaaaagaagagttaattaaagctattaatagtaatttttctttattaaataagaagctagaatctattacgccccaactcgcctttgaacctctattggaagggcacgcgaaggggactacgattagcgtagcgaatctggtttcctatctgattggctggggagagctggtgttacactggcatgaccaagaggcaaaaggaaaaactattatttttcctgaggaaggatttaaatggaatgaattggggcgtttagcacagaaattctaccgtgactatgaggatattacagagtacgaagttttattggcacggttaaaggaaaataagcagcaactcgtggctttgattgaacgattcagtaacgacgagctttacggtaaaccttggtataataaatggacccgaggtcgtatgattcaatttaataccgcctcgccttataaaaatgcttcggggaggttaaataaactgcagaaatgtcttgcagaatag

SEQ ID NO: 313 - protein, 244 aa, synthetic construct
  REGION 1..244: note = Synthetic: clbA
MRIDILIGHTSFFHQTSRDNFLHYLNEEEIKRYDQFHFVSDKELYILSRILLKTALKRYQPDVSLQSWQFSTCKYGKPFIVFPQLAKKIFFNLSHTIDTVAVAISSHCELGVDIEQIRDLDNSYLNISQHFFTPQEATNIVSLPRYEGQLLFWKMWTLKEAYIKYRGKGLSLGLDCIEFHLTNKKLTSKYRGSPVYFSQWKICNSFLALASPLITPKITIELFPMQSQLYHHDYQLIHSSNGQN

SEQ ID NO: 314 - protein, 3206 aa, synthetic construct
  REGION 1..3206: note = Synthetic: clbB
MDNTSGDFPCNKMDTRKQLPLTPSQQGFLFHSLKDKKRSNYHEHFTCIFSQHVDSAHFKWALETLFRKHECFRTDYNWEIDERPCQVVKTDVLPDIYVLDCEQEEIRFLLANDDIIIPVPQDDGIDAIIPQLLQADLKYPFSLKTIPVRAYLIQSTKESAFILSYHHIVMDGWSLSLFIKQLLQLYGAAVVSGVRDDSAIIPSSLKPLVDTLSARRHTFQHDYWAAYLREGTPTCIVPLSQYHTDTEAENNSYVNQTNHVEINLSPDVCQKIQTLCSDYRITPAVIFYVAWGILLQRWCYADDVLFGATISGRNIPIDGIEETLGLFINTLPLRLRDDGATLLQHLQRMHQTLIAHYSNEHDALASIQRLVHKEGHAGDLFNTLVVLENYPVDMTLLSCASPVAIRHLSVHEQTHYPLTLTITQQKGFRFSIAYALNYLTNNMAQALLMHLSYLLEQLVDNPQRPIAALVNLSPCQQAQVLQPYLERMACRDWDSQSNVIEQFHQVAATSPAQVAVVDELCALTYSELAAQAEQLAAYLVQQGVMVGDTVGIISERRVNTVVAIIAIMLIGAAYVPISPDYPVGRMQEIIDDSGLALLLVHGKPLDALNVAQSDLCAFPVAPSVVFPVITPDSRAYVIYSSGSTGKPKGIAVAHRGLLRLIQGDSPLKVESGETTLLTCPFEFDVSVFEMWSTLLNHGKLVLLSKQALLDINHIRRTIADEQVARAWFTSSLFNSYVAEGADFFGMLQHITVGGEAVSAWHVNDVMQKYPHLVVTNGYGPTENTIFTTAYRFNGLQPARVPIGYAVPGTSLYITDLHGHLLPIGATGELVAGGVGVAIGYQNNPALSATVFVPDPFIPGGMMYKTGDYARLLDDGCVDCFGRKDGQIKINGQRIETGEIEQRLLECSGIIEAVVVPYRVRETLHIAAVVCVNDSYDEVEVRGQLADRLPPFAIPESLVVVTEIAKSHSGKADLAQLRYLLPATQCNAVSTTISEVHSDMEHALHAIWQRVLDRQDIDSNASFFALGGTSLDTIRVKGDIKRQLGLEIDITDLFKYPTLTALAHFLDTAVSPEDAIPTRAVVYSDMPVAIVGMAGRFPGAANIAALWTLVVGGESGLTLFSDEELRAHGVTPDTLKQANYIKTKGIVDDHEWFDADFFGYTPNEAECMDPQIRLLHQCCWQTLEHAGCDPATFTGAIGIYAGLLTSPHWLNAVMQDTTDSTALYKASILNIHSVTALIAHALNLTGPAVTLDTACSTSAVAIHQACIALRNRDCDAALAGGVSIEMPAYRGYEYHEGMINARDGVCRPFDSQASGTVTGDGLGMLLLKRLDDALADRDCIYGVIKGSAVNNDGNNKIGYTAPSVIGQSTVIRTSLRRAGFDSDSIGLVEAHGTGTVLGDPIELRALNEVFGPTPVPFCVVSALKSNIGHLNSAAGVAGVIKTTLALHHQVLPPTAHFRQLNPAIDLSRSALYVNQQVQPWPSTRPRRALVSSFGIGGTNASIALEAHQHEDDPSATGVRDSYLLLFSAKTPAALELRVASTLEYVKHGVGVRLPDVAYTLQTGRTAFDHRRAYLVSRGSKIDLSCATILQAEIFNGQRTTAEICFMFPGQGSQYHGMASALYAHQPMFRQHMDRCFAAFQRYSTVDLKALLFDDEDTRDIDQTQFTQPALFCVEYSLARTLIDLGITPDSMIGHSLGEYVAACIAGVFTLEDALHVIEARGRLMQSMRPGSMMAVYLSREQLTPWLAAERGIELAANNSAHFCVVAGEQAAISRLSTRLVEGGIQHRRLKTSHAFHSAMMTPMLHDFAQLLGQIPMHAPHKRFISNVSGTWITEEQATSPDYWVQQVRNAVLFSEGAAQLLVQPTLFIECGPGNTLSTFIQGHNQYSDQPTLLTLRKANAAIDDEHMLHRTLAALWVRGENIDWRRFNQTALGKHIPLPDYPFEQTYYYRYGAALSGYRQYPNPLRRPQDEWLQRVLWRMHDTSLREAFYAPGELIIIISADGDKLQQTLMSSGVDSITMPLPISSEDDVWDNDRILTHFHDICALLAHKTYRQLHCLYAPGAEAGSSLTQSLSGLYRVARWCMHSTTPLASLTVLTHGAFRVQEEDNPEPTLAALSGAVNVFAQELHPTEVRLIDIDAQSSDENLNLLTQRLAPKQETVMALRQGMLYLRRFIPTRLLAHLPPQTGCIPGNVLWIIGGEKGIGRMIGEALAQREGVRVVLSSRTGYHHEAVQQDALDVIHCDVTQAEAVRACLATLLERYGRLDGVIFAADATTTLTLHQLSESALRDTLTVKERGTANVLHALAQRNLLDERLLLLFCNSLAAVNAEIGQTGYATASAYLDALAQQLRTRYKVNALSIGLDALREQGMLLDAINGSEYDVLRGLRPLMTGTLLQAYKQQGADTSYYARLSPESDWLLDEHRISGIATLPGTGYLALAYEALRHYFVQDQICIDELVFLAPLTVMDNCSVDVFVDISPNGQGVSVEVKSMTERFSGTLTTHARGRATRLMVDDNVVCDLTGLMREMHTITPPTKELSSTHFHYGPRWHSVQQLYGNTAQTQVFATLALPTVAANDTIALHPALLDIASSVVEQLPGFHTDSVPFLYQDLRLYRPLPNTLHVALTVNRHDEEGDSYAFTLYDMAGEMVARCAAMVKRKVQLHIQDVDDDTRLRVPSADNYQLRLAAEGEGAGKLALCPTPRLALGDSQVEIEVLATGLNFKDVLFTTGLLRQQPGEAPLQLGLECAGRITRVGKNVTEFAPGEDVMAVLNGGFVQYARVESDCVVRKPAHCRIEQAAALPIAYLTAYYALVVRANLQPGERVLIHSAAGGVGLAALHIAKRCGAQIFATAGSEQKRDYLLSLGVHAVADSHDEQFAATLLTASDGQGMDVILNSLTGRLLDASLALLAPLGRFLELGSKDIVEDKALPMRFFAQGGTFIPINFHAAHGAFSRYLQQIVAWIDDNTLPLLPCKSVPLPEVARAFATLTTPQHIGKVVVTHRTAAGMDRLNAMIAERRLGGYALSMSNAEVMRQLWPILNTRSPWAQLLLSPRAIDRLARGNRVDRGVPSAANDTITQQTVKKRPRPEIGVPYSPATREVERVLCQILEEYLGLDRVGIDDNYAELGATSLDMVQLSGQMARHYPQVSVVSLYNHATVRQLATFCQPPEGESNAPSPQPAVQTNTRANQIAKRALQIAKNTARSHTSLH

SEQ ID NO: 315 - protein, 866 aa, synthetic construct
  REGION 1..866: note = Synthetic: clbC
MEYASEMNGMEIAIIGMAVRFPQSRTLHEFWHNIVQGKECVTFFSEEELLAEGVEQSTLDNPAYVRAKPYIEGICDFDAAFFGYSHKEAQTLDPKSRVLHEVAYHALEDAGYAQRTSDLITGVFVGASEDVDWLRRSLSQIGGDALNRFESGIYGHKDLLAHLIAYSLNLNGPVYSLYTSCSTSLSATHIACRSLLFGECDLALAGGITIDLPQKSGYFCQQGMIHSTDGHCRPFDSQASGTLFGDGAGVVVLRRLEDALAAGDRIYAVIRGSAVNNDGKQKIGFVAPGHEGQKAVICAACHLAEVSPESIGYVETHGTGTRIGDPIEFAALTEAFDTSHRQYCALGAVKANIGHTHAAAGVAGLIKTALVLHHRTIPPLANYQMPNSKLDLAHSPFYIPIQPQEWPASRMPPRAGVSSFGIGGTNVHMILEGLNPAVRDDHDQVRAPVFIPLSAPSFEQLDELTQQLTPLLATLDASTLAYTQQVARPVFDCRRVIQVENDGTQAMLASLDNLMPDAPWGLHCPDLRTTNDCTYAQWLAHSAHYQREATALTALLDGMNIPPAYCHAETWAAQANSSLLIRGCQTIAALKTWMNLLPTLTLLSGAGTGLLPAAAASGMIATQDVLHLLWEMEQKALHLWLPERHEPIPGYVLAWQGNPITDAQRNDRGFWSEALLADTRELGEGVHSINWVRLPPEIREDVDVLRYVAQLWCAGINVDWAVWYGTPLPQRGSASAYPFAHNHYPLPGRVMGSVETQPEAGPETHHPYQARPVLSVPFVAAHSRGMQYITGLMELLLEISPVGVDDDFFELGGHSLLVTQLTSRLERDFNVHIDLLTLMENPNPRNIYAHIAAQLGGEDNLEIACQ

SEQ ID NO: 316 - protein, 289 aa, synthetic construct
  REGION 1..289: note = Synthetic: clbD
MMNVAVIGAGVMGTGVAHNMAQYGISTNVVDISQSQLDKCRQMIEANLRLYNFHPQHKKKTHSTAEIMENIRFTTELDDIVECDLVIENITEDIEKKNALYTRMNTICGASTVFGVNTSAISITALSKLMRHPENVVGVHFMNPVPLMHTVELIRGVHTAERTLNIFHHLFAQLNKTGIVVNDSPGFVTNRAMMIFVNEAIFMVQEQIARAEDIDTLFKTCFGHKMGPLQTADLIGLDTILQSLQVLYESFNDDKYRPSFLLKKMVDAGYLGVKSGQGFYRYQQTYAEQ

SEQ ID NO: 317 - protein, 82 aa, synthetic construct
  REGION 1..82: note = Synthetic: clbE
MKKQDMKAAIREFLSRSLRGHTLNDDDDIFSLGLVHSLFTVQIILFIEKNFQVELEVSELKTEQIATVNKIVELIQRQTGLE

SEQ ID NO: 318 - protein, 376 aa, synthetic construct
  REGION 1..376: note = Synthetic: clbF
MCTENYELAQQEAVLFAKQHLALAAQNIERQQFIVPDIISCVAQAGYLGASIPQKYGGRGYDSYQLCALHEVMAGVHGSLENLITVTGMVSTLLQRVGSAAQKAHYLPKLATGELIGAIALTEPNIGSDLVNVETELQQDGDGWRLNGKKKWITLGQIADFFIVLIHCGNQLATVLIDRNTDGFTITPLNDMLGLRGNMLAELHFNDCRLKEDALLGPLTPGVPLAVNFALNEGRFTTACGSLGLCRAAVDVAARYIRQRKQFKRRLFSHGIVQHLFATMLTQTRSAQLMCFSAAEYRETLHPAMINQILMAKYVASKAAVDVAGKAVQLLGANGCHADYAVERYYRDAKIMEIIEGTSQIHEIQIAMNYMMGSEA

SEQ ID NO: 319 - protein, 422 aa, synthetic construct
  REGION 1..422: note = Synthetic: clbG
MTKDVALMFPGSGSQYVGMARWLYERYPQVRTLFDEASQITERDMAALCLSGTLVQLAEPTAMALAIYTTSVAHFVAWQQFLAQTGVHVNLRYMLGHSLGEYAALTCSGALSFSQALALVAMRSRLASEIAREMDASTTIIKQGNQALVAAACEVAERETRQQVGIACFNSPQQFMLSGQNSAIIAAEQYLLDHDRQVEVVPLIGGVPYHSPLLKPCGQQLRKALDRCEWRRPCCPVISNVNAQPYPDTVTPVQWLEQQLSQPVQWQRSLTYLTGHLSPIAIEIGPQSVLKNLLLENRYPAPVYAFDNRHDRAQLALVLGDNMAVKTDPEAVRRQRITLLTNALTATRHHRAADVAASAQLKELLSRFFERIQQIEQRGTSSEEDIAFLHELLEQGFQLKGSSQAEIDACHARLASDNGGQA

SEQ ID NO: 320 - protein, 2310 aa, synthetic construct
  REGION 1..2310: note = Synthetic: clbH
MEQQGIMRQLPTDDQTIVDYLYRIAGEYGEKAAVLMGDAALSYHDLNARSNQLAHYLRGLGIGEDRVVAIRLPRGMAMLIAIFAIVKAGGAYLPLAYNAPRSRIENILSNSGAVCLIGTDDGDRWPIPRVEIDSAAVSAMPTTDLRYRPHARQLAYIIYTSGSTGVPKGVATEHAALLNRIVWMQNAYPISSQDVLFQKTVYTFDVSVWEMFWWAMYGASVVLLPSGLESDPRTLARLIQRHRVSVVHFVPSMLNLFVEYLEMKQDPRLTASLRLVFSSGEKLTVHSVARFYQSVAQGDLINLYGPTEAAIDVSHHRCLRGYDYDDIPIGQAIDGCRLYVLDDHGNPVADGEEGELYLAGIGLARGYLNNVALTDRCFTIHPTLRHLGKPERLYKTGDLVWRDGESQQIHYIGRNDFQIKIRGLRVELGEIEAHAMRFPGVQQAVVVADQDDPDNQLIYAFVVSSVPLNLAALMDALSKNLPAYMLPNRLLAMSELPLSDNGKCCRKTLLDLARAYSASRVDLRETPAVRYLPLSSAQSSMWFMQQLAPHTALYNNPTALLLEGELDRTRMDGAIRQLMSRHTLLRAMAETHNGQPVLAVPQCVSSQALLTIVPLPSVSDDNALQAMINQRAAHPMPLTSGTPLCRFELLTLDDDRSVLLIHLHHIISDGWSKGVLLRELQAAYNGESLTPEPLLEYADYMEYQEEWRQSDAYQDAMRYWQNTLAGTLPILDIPTDQPRQKVARYQGAFVAFALSANTCERVLAAARAQRVSLYNYLLTAFVLLLHRNARQQEYIVGMPIAARLTKEQEHMIAPLVNVLPLRLPLDEAASFSELVQTIRGILFAAFRHQRLEFTDIVRAVNVDRSAGTPCALLPDMQAYLLFTSGSTGEPKGVCVVHRGLLNLLLDMQRTFAVGSQDRLLSVTTPTFDISFLEYLLPLISGASLYLTEAERAADSFRMIPLIADYRPTLMQATPSFWHGLLMAGWRGDPELCVLAGGEALPTKVAEELLRCCGSLWNLYGPTETTIWSLKSQITQAENITLGAPIANTRIYILDNEGHPVPQGVDGELYIAGDGVAQGYDGQPELNAQFFLSEPGVPGGRMFRTGDLVRSDAQGQLFFVGRKDSQIKLRGYRIELGEIERTLARHPHVDAAVVACIERAPLHKALAAFIITSEPPSLFEQLKNELRQQLPDYMVPTLWQRVADFPNTDNGKIDRKRLAENFVADSSLVSPQTQALSDTEQMLLALWMRYLPIKNVDPECDFFRLGGHSLLAVTLVAEINRTFHCALTLKDIFHYSTLRALSARIAQQSITDAAASQDDWVIVHDPEHRHQPFPLTDVQRAYWLGRQTGATSIATHIYHEFDVEHFNVTRFTHAVNALIARHEMLRARVLPDGTQQILAQVPAYQLEQRDLSALSPNARNDALMAIRDRLSHHVHPADRWPLFDFSYSACTAQHGRLHFSLDLLIADALSMRTLQQELMMLYREPHVSLPLLPFSFRDYVQALLVEQASEAYARDQAYWQRALPQLYGPPTLPVQGDLAQLSAIRFVRRRHRLSAHNWGVLSALAQRTRITKTALLLTVFHFPIYQCMFQLDNMPLASPTLNGVNVTPLLLDTSASQVDISLSMQHIDGRITGTFEYDAGLYSADRIQHLVAQWRELLDEASSQPTQLVRDLIRFTPREHAWLARHNATEVALPPVDNLLALVLPHCQQRPTQVALRHADDAMTYGELQQATMQMCTWLRAQGVKRGESVALQLPFCFELIIAQLAILSLGASYVPLDGNAPAARNALILAQATPCMLLVAQPLESPHGLTIPWVLVPDWRSLLTEIPNLPVSVAPDALDCDAVVIFTSGTTGQPKGVRLSQRNLVNLTASFISSYQVTHQDVLLPITSVASASFVGEVLPLLAAGGTLVLAQKAQSLDSDALIALLASQRVTILSTTPSLSASLSVLAQSMGSLRLFLCGGEALEYEQIAPLLPHMAVVNGYGLTESGICSTYFPVAKRREQETGALPIGRPIQNTQAYVVDAYNRLVPPGACGELCFSGLGISPGYLDARQDPERFVELPEYPGVRVLKTGDRARWATDGMLFYLGRQDRQVQIRGYRVELGDIESLLKQHPDIADAWVDVRRNAAATPLLVAFYCSVNGVALDAQQLRVWLSLRLPLHMLPLLYVPLSAMPLGVNGKIDPQCLPLVDLRQLEGPGEYVPPATELEQRLAEIWQQLLGLERVGTTTNFFDLGGHSLLLVQMQQYIGQQCGQHVALVDLLRFTTIKRLAEFLLAPDAAQGTTGDQTQLRAAKQRLAFGHTRWAATTDSHH

SEQ ID NO: 321 - protein, 1010 aa, synthetic construct
  REGION 1..1010: note = Synthetic: clbI
MAENDFGIAIIGMAGRFPQADTVQAFWENLLASRECISFYSDEELLAMGISPEFVQHPDYVKAKGEVADIDKFDAAFFGIAPREAELMDPQHRVLLETAWAAFEDAGYVAADYPGDVGIFAGKSMDSYLMLNLMPHFKRVFSSGSLQAAIGNDKDSITTTIAYHLNLRGPAITVQTSSSTSLVAVCVACQSLLTWQCDMAIAGGVTLGPPAKTGYLSQEGGITAADGHCRAFSDNSSGFVPGTGAGLVVLKRVDEALRDGDNIYAVIKGFAVNNDGSEKISYTAPSVDAQARAIAQAQRLAGLTPQDITYVEAHGTGTRLGDPVEFSALSQAFAGASQKQYCALGSVKTNIGHLDTAAGVAGLIKTALAVQQGIIPATLHFERPNAQIDLTNSPFYINTTCQPWQPESGIRRAGVTSLGMGGTNAHVVLEQAPAVDLQARAPVPAYSILPFSAKTDSALSSGLARFADFLQHESLPDRRDLAWTLSQGRKAFAHRAALVTRDLHAAGTLLQQAATAPFARGVAQTQLGLGLLFSGQGSQYQRMGHQLYQVWPAYADAFDRCATLLEREYQLDIRHELFRAEVSLAQGERLAQTCLTQPLLFSVEYALAQLWLSWGITPTVMIGHSLGEWVAATLAGVFSLEDALRLVARRAELMHQAPSGAMLMVALPEAQIRALITAPLAIAAVNAPDYSVIAGPTSEILAVSQRLTEQNIINKRLHTSHAFHSSMMQDAAQALRQAFENVRLNPPTLTIISTVTGAHVSADTLTTPDYWIEQMLMPVQFSAALQEAQATFDVDFLEIGPGATLTQLTNGHALGDRLAFSSLPAGARSSDEHKHILDTVAALWVRGHNIDLSAFAGEQPRRVSLPTYAFDKIRYWVDSPEEQRSAVTPVADAGSVIPSEPSVRRQPRPAFSVPYAAPESKTQRGLVAICEALLGIDGLGIDDNFFEAGGHSLMLGMLLAQVQERFAVTLSFFDVMEDASVRALAQLVEQEQQDDGGSALAVLVNDMINE

SEQ ID NO: 322 - protein, 1454 aa, synthetic construct
  REGION 1..1454: note = Synthetic: clbJ
MTIHHAALARMLPAEKKEKLLRQLAQSGVSPSRIPIIKADPAQAIPLSFNQERLWFLQKYDSTATNYNLYVVYRLHGVVDMPMLTEALRHVQARHAILRTRIIVRNDRPCQVIDDASSLVLDTVTLAAQAPTSALDAVIQQVINTRFDLARGPLWGVTQIIQPDQGCHLVFCAHHIIIDGISLRLLFDELQQQYARLHAGNETSLPPPPLQYADYAFWQREWFQDTLLANELAYWRARLQDAPLLSTFPSLHPRPAQPSTHGSRFSITLDETLSLALKHVARTQETTPFVLMLTAFQLVLMRYAQQQRLVIGMPVSGRIRPELQSSIGYYASTAVIYTDFNGVEVGREALQRVKASVKETQGRQQLPFENLVNMLDLPRSLSHSPLFQILYIYHNHVTPRAFTLAGAYWEQVTYHNQTVKYDMTVEVFQNDATFDVSFEYDLGLYDADVVKQIAEALRQHCLSLTSSLETPIGAIPLHAPETATPRRDPLNATNVPWLGPQDVLRIIEQRCVQHPKQLAIQQHDGTLTYAELWARVQFIAMRFRAHGIQPGDRIGVLLPRHRDVIATMLATWFVGACYVPFDIHQPAARLQRLMQRARLVCLVVRQPGEWGEIVQLSLPELMQDMSNAIRYSSQVLARWSLSPTFTLNLTLFNRPQGYPNAEAVIGDFTAVSLLNVCYDSQHSYAHNAQRIQVQLWEDLEHRRFSGIRASEALIHSGRFHAPMPVVFTSMLDIDGETTAQDPRDTTRFTLCPDANITQTPQVWLDHQVIELAGELHFNWDAVEQLFDTTLLDQMFGAYCHALQALVAMPQSWWGVNSSLALPTVSAPVTQAPAPTALLHHGLLRQAALTPQETALISPIRELTYRQLSTAADHVARALLALGVQHGDRVAVVMEKGWQQIAAVHGILRLGAVYLPVDPVLPPQRRQLLLTVGEVRVQVTQPGLTQLEPSLPVLIIDDGMLDTPAAPLPEVAGDVTDLAYIIFTSGSTGTPKGVMIDHRAAMNTLEDINERFGLNAQDRVFGLSSLSFDLSVYDAFAPFMVGAALVLPEAGREKDPRHWQTVMAHGHVSVWNAVPALMQMLCEYHSGDRMSYPTLRLALLSGDWIPLTLPEQMRERLNETMDIISLGGATECAIWSVYYPIGEVESTWTSIPYGRGLRNQPVYVLNAQLEECPVGVEGEICIGGMGLAQGYLNDAEKTAASFVWREASGERIYRTGDRGRYFADGQVAFLGRNDTQVKVNGYRIELGEVKSHLEQLDSVGSAAVVCHQGQLYAFITAAENLHPDDTDALLARVRAQLAVQLPYYLLPQHFFLLKVLPMTGNGKIDQAAMVQEVIQRMSQSTSQKSRALAHASPYEQQVAALWCEVLQREQIGLNDNFFEAGGGSIQIVLLHRRIEEIFKVTVPIAELFRLTTVKRIAGYLQAMQDNARAVNQTQQRDASRSRAQQRLVRRHQRQR

SEQ ID NO: 323 - protein, 2154 aa, synthetic construct
  REGION 1..2154: note = Synthetic: clbK
MTYSESDIAIVGMNCRYPGVHSVAAFETVLRTGCNILDPKVTPSNGHNHITLNNVYEHMAEFDANFFGYSRAEAEIMDPQQRVFLTCAWEMFEQSGYNPKQHDARVGLYAGVSTSFYLLTHLMNNPDKLAQLGGLQIMVGNDKDHLTSQLAYRLNITGPCVTVQASCATSLVAVHLACEGLLSGQCDMALAGGVTFRMEEQRSYESHGDGLQAEDGLIHTFDAQASGTVYSSGLGMVLLKRATDAQVQGDNILAVIKGSAINNDGGARSGYTVPGVDGQEAVMIEAHSLAEVTPQQIQYLELHGSGTPLGDAIEFAAIKRVFGTPAPNATPWRLGAVKPNVGHVEMASGITSLIKTVLSLTNRVFYPTLNFQRANPQLGLEDSPFEVVSRLTPWPEGTTPRTAGVSAFGLGGTNAHLVVQAPLSTPQARAQQMGPCVVVLSAKNHNALEQMQNALLAKLAAHPEIRLQDVAYTLRHGRFSAPVRKCVIAENCTQLARQLRDAPMVEATTGCTIYWRLGHRFVVALETLSDWLACSEVLSQAVGQLLEHFPLEPACLQDLSPAQRTFISQYALIALIDERETLNVVLCGDGDGGYAAAVLRGDCTLEQAWHRLNAGQPFDDVPTNPLLQPDVCSLMLDDAASDANRTALEALGQLWLAGVSLDWRWVDAAERMLGSQRIALPGTVFTPQRYWVEAVRPATFSHESSNNLLSRATKSDIIAVVTEIWERTLGVSIDDHHASFFELGGHSLLASTILYDIQQRYGITCTLSAFFADPTIEGLSCYLLEQGGSETAVSALPDTVFAPDQQHLPFPLTDVQQAYWVGRRKSLGLGNISTHIYVEYELQGLDETAFNRALNAVIARHSMLRAIVNDDGMQQILPNVPEYHVAFYTTQCEDAFQQRCRELRDTLSHQMIDCSRWPLFQMEVVVDPQQKARLHVSIDLLIADAWSLELFIRELAYHYRHPQAALPTLTYSFRDYVLTLKSYEKTPQFERARDYWRARIETLPPGPRLPLRTDPTKLENPTFVRRSYCLSRAIWQRLKTQAGQMSITPTTLLLTGFAQVLARFSSSPHFSLNLTLFNRLPLHADINHLIGDFTALTLLEIDMSQGETLQARANVIHSQLWRDLDNRLFGGIQVSRLLVQTHRDPAKSVIPIVFTSLLNQYEASWETDDTLFNQPQDDLYSISQTPQVWLDHQVMERNGELHFNWDVVEQLFEPALMDQMFQCYCQLLHALAQRPQLWHETQDVLALPTVSAPVTQAPAPTALLHHGLLRQAALTPQETALISPIRELTYRQLSTAADHVARALLALGVQHGDRVAVVMEKGWQQIAAVHGILRLGAVYLPVDPVLPPQRRQLLLTVGEVRVQVTQPGLTQLEPSLPVLIIDDGMLDTPAAPLPEVAGDVTDLAYIIFTSGSTGTPKGVMIDHRAAMNTLEDINERFGLNAQDRVFGLSSLSFDLSVYDAFAPFMVGAALVLPEAGREKDPRHWQTVMAHGHVSVWNAVPALMQMLCEYHSGDRMSYPTLRLALLSGDWIPLTLPEQMRERLNETMDIISLGGATECAIWSVYYPIGEVESTWTSIPYGRGLRNQPVYVLNAQLEECPVGVEGEICIGGMGLAQGYLNDAEKTAASFVWREASGERIYRTGDRGRYFADGQVAFLGRNDTQVKVNGYRIELGEIERCIARHPDVEQSVVVAVGNSQHRRLVAFAKLHDRHQAQALQAKEAEAAALAQGIIVNPAQRLAFKLKEPHIRALDGLGIALTAPADSTRYIKRRSYRHFSAQKTTLAQLGQLLSGLGQMRLPGLPFAKYAYASAGGLYPVQTYVYLHPDKIEEGVSGIYYFDPRQSCLMPVAPEVELNSGFHAGPNQSIADRAAFTLFMVADMAVISPFYGQEAAWHFSVMEAGTLCHLLEEDAPRYGLGLCQLGMADFSAVASHFQLSPHHRYVHCTVGGAIGQEAASAAALLRDFSTYEKPKETAAPLDMQSYKDAMLRGLRQQLPDYMVPSDLMLATDFPLTANGKLDRQKLQLQGEQIAHQRDGVGPIQVDSALQQRLVALWQEVLGVSHVSAEDDFFSLGGSSIELVRIQQALEAIIGQEIPIVDLFRLPTIADVARYLDEQLHNLPAAHDIVLAQAEVSQVSAARENLALRRKRAQQGEKGDE

SEQ ID NO: 324 - protein, 487 aa, synthetic construct
  REGION 1..487: note = Synthetic: clbL
MSEQSYRSAGTLLAQLASGETTSVALVNHYFSRMAQFNKPLNAVVQQHYALALEAAARADRERLEGRARGVLHGLPCTVKESFDVQGWLTTSGAHYLKDNRATQDAPSIARLRAAGAILMGKTNVPMMTADWQTYNDLYGTTHNLWDRQRSPGGSSGGAAVAVAADFTPVEFGSDLFGSLRIPAHYTGVYAHRCSLGLMSVRGHVPGGGPQATDEPDLSTAGPMARSAADLRLMMRALSTFWVEPPRIPDFSRYQAKANYRVCTWFSAPHHEIDQQIAQRFQSFIDKLRAQPGVEVDDAMPADIDPDALFDIAVKLSGRLVSTALNGRQRLTAGLAALGFRLVGKLADVPEGITSYYQGMLKDSGEQRNTDKLRHEYSRVIETLFARYDVLLTPVSPVLAFAHMQQPVRKRKLIVNGEPQDYNEHLFWNMLATVFGLPATVYPLAKTMDELPCGIQIISGHFHDDVTINFAEFCESISGGFTVPEGY

SEQ ID NO: 325 - protein, 479 aa, synthetic construct
  REGION 1..479: note = Synthetic: clbM
MTVNKLEPDNGTPNDELFTVAGMFDGSLYKLLLRMALPMFVGMLTQVTYAIADIFWLSHIDVTNSGIIAGVGLVFPVGMGLFAIANGIQIGMGSLLSRAIGMQRLDRAQRILSVGIIIALFFAIVITVLGYVYAQPLLRSLGATKSIIGYATEFYYYSLLTVFSIMLIGVMMGLFQGAGKIMVIMKASLLGALVNIMLDPIMIFVFDFGVKGVALASFLAQLSMVAYFIYTLMGLHIGLSIRIALRPFSWKIYREFLSVGMAQMLMQLIIAVGIVIYNFFIVRLDVNAMAAFTLTGRIDYFIITPMLAIATALLTVVGQNWGHGNVTRTLNAYWAAVALAFSIVLVLAVMHIVLAPWMYPLFTRVVAVSDYAVLQTRIMALALPFVAISLLASEYYQAIGKPWYSVLLTLMRHVFISVPVVYLLAIVLEMRITGVYFGAMSGTFVAALLAWRLLRLSPRLLRWNQEAVRSQHLDMEVAP

SEQ ID NO: 326 - protein, 1455 aa, synthetic construct
  REGION 1..1455: note = Synthetic: clbN
MMSGNPLSWPQEQCHIIDQLYPYSAVNIIGGVVTIEGIVDLPRLHAAIQSAIRQFDALRMWFVMGEESEVVSQVQPYHWRDIRHLTFSPDYDKENLRPAAIETFVDEWFRQPFTLLAHDLFEFVTFTCGEQYSGYLFKAHHGIADGWSMALLSNHVKRAYEQQDVPDDASPAYSAFLAQQQSYQASTRFAVDRGWWRDYIDEYRDCFPDSSPIVTTEGISCSTWLEPAMINRLYRLCNRYGCTLNTLFIALFALYRARVWGEEKGVIGVPLANRHTREARRCFGMFTNQLPLAYRLVRTERFCERVAFFQRELKRGFKHSKYPITLFNQDLAEQGGGKLRAFDYCVNYYNFTYERHIAGAAQRVESYYSGEQSYKLQIVLQTVNNHKESLRLSLEALRSAFTPHQLTAMKNGLLDLVTALDRQPDARLGDLEVYPAPHVALACGSLKPSFTSRFAAQVVEHGDRTALIDNEQSLTYRQLDDAVERVARYLRQQGIGRGQVVGIIAEHSAQTVMVIYGILRCGAAFLPLNPALPTTRLYAMCRKAQVAHILYDPAMHELTQALAFPASSLLQALATSALAREPWPAIEPQDLAYVLFTSGSTGEPKGVQVSHGNLANYLHFAAERYFTAQDRAALYSSLSFDLTITTLFAPLCVGASISVCRHAESETLLRMAVVDQPNTVIKLTPAHLRLLCAAGISSEQIRTLVVGGEDFKRDLARKAAALFPQAVIYNEYGPTEATVGCMIYRYTGQETLPSLPIGMAIDGCQVAICSPWGCPVPEGETGELVIYGASVTQGYIDAPQQTAAAYLKDTNGVMIGYRSGDIGYAIAPNTLVYQGRKDDQVKINGYRIELCEIEQALLSAPQVESAAVAVIDDVQGQHSGLLACVTPSSVDVATVMQHLRQQLPTYMQPKQCCAIAQLPLSHNGKVDVRQMVATVRNTAPASGSERLGDAAIRHSVRVCVEGALEQTEFDDNENLYVLGLDSIKSIQIAAQLRHHGWTMSAVQVMECGTVNAICEFLASHTTVSQLAQYAHNTRIDLPALRWFTQLALPVPNVYNHVIVLKVLPGCPLEQLHNRLHTLIQQQPALHSALDAEGRLLVCDPNVCYPNEVLTEYSTAQWTLAEVIAQCNSMLDVTNGRVFTAALLHAPQPASSTLVLCAHHLCVDMHSWYLILSTLDAVSTVNGTSNSGLHRWNDYLASKTVDSATHESWRTVCQTLPLHFPPVSLPDDSLPRTRAWREDFRHPCVRRLFESSGNTAYSAETYVLTALALVLRYYSEEPWCRIEMEGMGRGCWPDEPDVADTVGWFTLFYPWAIPLHGDMATLLSAIASDLAKRTHGGGDYGLLQMRHAPEDSLAQGIRMNYIGVQAQPSLRYFHIDHFNSDIYTAPENALGCVLEFNIARSAADGLSFHCRFDPTRIALNDVQLLLARYKNSLTDLDAWLCQHSATLTGAPTLWTL

SEQ ID NO: 327 - protein, 819 aa, synthetic construct
  REGION 1..819: note = Synthetic: clbO
MAKDDFTCGSLDIAIIGMSGRFSGAESVPEWWDKLLAGEEFTQPTCTEDDNGNPWIRLRNIITAPYDFDAAFFNIPPGEALLMDPQQRIFLECCYNALEHAGYIPTQLKRVGVYGATYANNYFIDRVYPYLKMSGDHHYLQAQIGNEKDYLCAQVAYKLGFTGPAVSVQTACSSSLVAAYLACEGLLTFQADVALAGGVTLGFLQAHGYSPQGDKLVSQDGHCAPFSAEATGTVYSSGAGVVVLKRLEDALRDQDRVYAVIKGGAVNNDGGRRLGFVAPSVEGQVEAINTALAAAEVVPTDIALIETHGTGTPLGDEIELEALHRVFAPACAPHSIQLGAVKANLGHLGVASGIVSLMKTALTLYTGLVPPQINLVNKHKKLLQPASPFYLSDVVTSVPQTKRIHATVSSFGLGGTNAHLVLQNWCETPAQAVQENERRLFFFSAKTPLALRQQLDAHYHALATYAEADKDRIAYTLAQRRAHFPYRCALAADSVVALRASLAKLRDADMSFTPINMETTLVFLYPDRDDKLESALTHLLACQPNLRQRHQRLSQDVAQICEPADWTPALRQFIQQVSLSEWLIEQSISPVQHIGYLTGAAAAQYVARIISLENAVQQVIVAETTPEQTLAGNSELSEILANLAVTEGTLMLEIGRAGTFSILYHQHAQWVGQTVFSPMLNTDTPEDILPLLGTLWQRGVTICLPEMPAVQTIGLPGYSFDRVRYEIQSSDARENAMLPVSYLSVSDFVEKTWRSLLCIDHYDEHAVIFEYGATSMHVISFVDSCNHIYKIGLTAADIYARPAIREHSEFISECVDGIL

SEQ ID NO: 328 - protein, 504 aa, synthetic construct
  REGION 1..504: note = Synthetic: clbP
MTIMEHVSIKTLYHLLCCMLLFISAMCALAQEHEPIGAQDERLSTLIHQRMQEAKVPALSVSVTIKGVRQRFVYGVADVASQKANTLDTVYELGSMSKAFTGLVVQILIQEGRLRQGDDIITYLPEMRLNYQGKPASLTVADFLYHTSGLPFSTLARLENPMPGSAVAQQLRNENLLFAPGAKFSYASANYDVLGAVIENVTGKTFTEVIAERLTQPLGMSATVAVKGDEIIVNKASGYKLGFGKPVLFHAPLARNHVPAAYIHSTLPDMEIWIDAWLHRKALPATLREAMSNSWRGNSDVPLAADNRILYASGWFIDQNQGPYISHGGQNPNFSSCIALRPDQQIGIVALANMNSNLILQLCADIDNYLRIGKYADGAGDAITATDTLFVYLTLLLCFWGAVVVVRGAFRVYRATAHGPGKQQRLRLRVRDYIIALAVPGLVAAMLYVAPGILSPGLDWRFILVWGPSSVLAIPFGIILLAFVLTLNHQIKRILLHNKEWDDE

SEQ ID NO: 329 - protein, 240 aa, synthetic construct
  REGION 1..240: note = Synthetic: clbQ
MSNISLYCLPYSGGSAAMYYKWRSVLSDNITLRPLEPAGRGTRIRQPLCLTMVDAVADLYQQFVKHYTGGDYAIFGHSLGGIMAFELVHYILDHGHDMPCALFFSGCRPPDRASHEVILHTLPDQAFMEEIVKLGGTPVDVFRNKELMTIFTPIIKNDYRLYEQYVFQAKARTLTCPIVLFHGDADNLVMQDELLAWEKFTTRKTRTIIFPAADHFFVDKHFEQVVGYVNQTIESLEIVG

SEQ ID NO: 330 - protein, 70 aa, synthetic construct
  REGION 1..70: note = Synthetic: clbR
MDKFKEKNPLSLRERQVLRMLAQGDEYSQISHNLNISINTVKFHVKNIKHKIQARNTNHAIHIANRNEII

SEQ ID NO: 331 - protein, 170 aa, synthetic construct
  REGION 1..170: note = Synthetic: clbS
MAVPSSKEELIKAINSNFSLLNKKLESITPQLAFEPLLEGHAKGTTISVANLVSYLIGWGELVLHWHDQEAKGKTIIFPEEGFKWNELGRLAQKFYRDYEDITEYEVLLARLKENKQQLVALIERFSNDELYGKPWYNKWTRGRMIQFNTASPYKNASGRLNKLQKCLAE

SEQ ID NO: 332 - DNA, 17 bp, synthetic construct
  misc_feature 1..17: note = Synthetic: Operator 1
  source 1..17: mol_type = other DNA
taacaccgtgcgtgttg

SEQ ID NO: 333 - DNA, 17 bp, synthetic construct
  misc_feature 1..17: note = Synthetic: Operator 2
  source 1..17: mol_type = other DNA
tacctctggcggtgata

SEQ ID NO: 334 - DNA, 169 bp, synthetic construct
  misc_feature 1..169: note = Synthetic: FNR responsive promoter
  source 1..169: mol_type = other DNA
agttgttcttattggtggtgttgctttatggttgcatcgtagtaaatggttgtaacaaaagcaatttttccggctgtctgtatacaaaaacgccgcaaagtttgagcgaagtcaataaactctctacccattcagggcaatatctctcttggatccaaagtgaacccgc

SEQ ID NO: 335 - protein, 8 aa, synthetic construct
  REGION 1..8: note = Synthetic: Description of Artificial Sequence: Synthetic peptide
GGGSGGGS

SEQ ID NO: 336 - DNA, 478 bp, synthetic construct
  misc_feature 1..478: note = Synthetic: nirB promoter fused to ribosome binding site
  source 1..478: mol_type = other DNA
atttcctctcatcccatccggggtgagagtcttttcccccgacttatggctcatgcatgcatcaaaaaagatgtgagcttgatcaaaaacaaaaaatatttcactcgacaggagtatttatattgcgcccgttacgtgggcttcgactgtaaatcagaaaggagtcagcataacaccctgacctctcattaattgttcatgccgggcggcactatcgtcgtccggccttttcctctcttactctgctacgtacatctatttctataaatccgttcaatttgtctgttttttgcacaaacatgaaatatcagacaattccgtgacttaagaaaatttatacaaatcagcaatataccccttaaggagtatataaaggtgaatttgatttacatcaataagcggggttgctgaatcgttaaggatccctctagaaataattttgtttaactttaagaaggagatatacatgaaaacacct

SEQ ID NO: 337 - DNA, 200 bp, synthetic construct; identical to SEQ ID NO: 12
  misc_feature 1..200: note = Synthetic: fnrS fused to a crp binding site
  source 1..200: mol_type = other DNA

SEQ ID NO: 338 - DNA, 14 bp, synthetic construct
  misc_feature 1..14: note = Synthetic: RBS
  source 1..14: mol_type = other DNA
gctggaacaggtgg

SEQ ID NO: 339 - DNA, 14 bp, synthetic construct
  misc_feature 1..14: note = Synthetic: RBS
  source 1..14: mol_type = other DNA
tccggaagactagg

SEQ ID NO: 340 - DNA, 60 bp, synthetic construct
  misc_feature 1..60: note = Synthetic: PhoA secretion tag DNA sequence
  source 1..60: mol_type = other DNA
aaacaatcgaccatcgcattggcgctgcttcctctattgttcacaccggtgacaaaggca

SEQ ID NO: 341 - DNA, 99 bp, synthetic construct; identical to SEQ ID NO: 174
  misc_feature 1..99: note = Synthetic: ECOLIN 19410 Tag
  source 1..99: mol_type = other DNA

SEQ ID NO: 342 - DNA, 21 bp, synthetic construct
  misc_feature 1..21: note = Synthetic: Description of Artificial Sequence: Synthetic oligonucleotide
  source 1..21: mol_type = other DNA
ggtggtggttcgggcggtggg

SEQ ID NO: 343 - protein, 15 aa, synthetic construct
  REGION 1..15: note = Synthetic: linker
GGGSGGGSGGGSGGG

SEQ ID NO: 344 - protein, 30 aa, synthetic construct
  REGION 1..30: note = Synthetic: linker
  VARIANT 1..30: note = This sequence may encompass 1-6 "GGGGS" repeating units
GGGGSGGGGSGGGGSGGGGSGGGGSGGGGS

SEQ ID NO: 345 - protein, 9 aa, synthetic construct
  REGION 1..9: note = Synthetic: linker
GGGGSGGGS

SEQ ID NO: 346 - protein, 7 aa, synthetic construct
  REGION 1..7: note = Synthetic: linker
GGGSGGG

SEQ ID NO: 347 - protein, 8 aa, synthetic construct
  REGION 1..8: note = Synthetic: Description of Artificial Sequence: Synthetic peptide
HHHHHHHH

SEQ ID NO: 348 - protein, 7 aa, synthetic construct
  REGION 1..7: note = Synthetic: Description of Artificial Sequence: Synthetic peptide
  VARIANT 4: note = X is any amino acid except P
VPGXAAG

SEQ ID NO: 349 - protein, 5 aa, synthetic construct
  REGION 1..5: note = Synthetic: linker
GGGGS

SEQ ID NO: 350 - DNA, 326 bp, synthetic construct
  misc_feature 1..326: note = Synthetic: lacI
  source 1..326: mol_type = other DNA
attcaccaccctgaattgactctcttccgggcgctatcatgccataccgcgaaaggttttgcgccattcgatggcgcgccgcttcgtcaggccacatagctttcttgttctgatcggaacgatcgttggctgtgttgacaattaatcatcggctcgtataatgtgtggaattgtgagcgctcacaattagctgtcaccggatgtgctttccggtctgatgagtccgtgaggacgaaacagcctctacaaataattttgtttaaaacaacacccactaagataactctagaaataattttgtttaactttaagaaggagatatacat

SEQ ID NO: 351 - protein, 8 aa, synthetic construct
  REGION 1..8: note = Synthetic: anchor
AGYGSTLT

SEQ ID NO: 352 - protein, 6 aa, synthetic construct
  REGION 1..6: note = Synthetic: HIS tag
HHHHHH

SEQ ID NO: 353 - protein, 20 aa, synthetic construct
  REGION 1..20: note = Synthetic: linker
SGGSGGGGSGGGSGGGGSLQ

SEQ ID NO: 354 - DNA, 60 bp, synthetic construct
  misc_feature 1..60: note = Synthetic: linker
  source 1..60: mol_type = other DNA
tccggagggagcggcggcgggggtagtggaggcgggtcaggcggcgggggctcacttcag

SEQ ID NO: 355 - DNA, 36 bp, synthetic construct
  misc_feature 1..36: note = Synthetic: Flag tag, Xa site
  source 1..36: mol_type = other DNA
gattataaagacgatgacgataagatcgaaggacgt

SEQ ID NO: 356 - DNA, 22 bp, synthetic construct
  misc_feature 1..22: note = Synthetic: linker
  source 1..22: mol_type = other DNA
tggtggtggttcgggcggtggg

SEQ ID NO: 357 - protein, 15 aa, synthetic construct
  REGION 1..15: note = Synthetic: linker
  VARIANT 1..15: note = This sequence may encompass 1-3 "EAAAK" repeating units
EAAAKEAAAKEAAAK

SEQ ID NO: 358 - protein, 20 aa, synthetic construct
  REGION 1..20: note = Synthetic: linker
  VARIANT 1..20: note = This sequence may encompass 1, 2, or 4 "GGGGS" repeating units
GGGGSGGGGSGGGGSGGGGS

SEQ ID NO: 359 - protein, 18 aa, synthetic construct
  REGION 1..18: note = Synthetic: consensus sequence for binding
TTGTTGAYRYRTCAACWA

SEQ ID NO: 360 - DNA, 15 bp, synthetic construct
  misc_feature 1..15: note = Synthetic: binding box
  variation 8: note = n is a, c, t, g, unknown or other
  source 1..15: mol_type = other DNA
ttataatnattataa

SEQ ID NO: 361 - protein, 108 aa, synthetic construct
  REGION 1..108: note = Synthetic: LARD3 Secretion Tag
IEGRGSDGNDLIQGGKGADFIEGGKGNDTIRDNSGHNTFLFSGHFGQDRIIGYQPTDRLVFQGADGSTDLRDHAKAVGADTVLSFGADSVTLVGVGLGGLWSEGVLIS

SEQ ID NO: 362 - protein, 60 aa, synthetic construct
  REGION 1..60: note = Synthetic: HylA Secretion Tag
STYGSQDNLNPLINEISKIISAAGNFDVKEERSAASLLQLSGNASDFSYGRNSITLTASA

SEQ ID NO: 363 - DNA, 63 bp, synthetic construct
  misc_feature 1..63: note = Synthetic: OmpA Secretion Tag
  source 1..63: mol_type = other DNA
atgaagaaaaccgcaattgcaatcgccgtcgctctggcggggttcgctacggtcgcccaagcc

SEQ ID NO: 364 - DNA, 63 bp, synthetic construct
  misc_feature 1..63: note = Synthetic: PhoA Secretion Tag
  source 1..63: mol_type = other DNA
atgaaacaaagcactattgcactggcactcttaccgttactgtttacccctgtgacaaaagcg

SEQ ID NO: 365 - DNA, 327 bp, synthetic construct
  misc_feature 1..327: note = Synthetic: LARD3 Tag
  source 1..327: mol_type = other DNA
attgagggccggggttctgacggtaatgatcttatccaaggcggtaagggcgcggacttcattgaaggcggcaaaggtaatgatacaatccgcgataactccggtcacaacacctttttgttctcagggcattttggtcaggatcgtattataggatatcagccgaccgatcggctggtattccagggcgctgacggcagcacggatctgcgcgaccatgcgaaagccgttggagcagatacggtgctgagttttggcgccgattcggttactctcgtcggggtggggttaggaggcctgtggagcgagggtgtgctgattagttaa

SEQ ID NO: 366 - DNA, 183 bp, synthetic construct
  misc_feature 1..183: note = Synthetic: HlyA Tag
  source 1..183: mol_type = other DNA
tcaacttatgggagccaggacaatcttaatccattaattaatgaaatcagcaaaatcatttcagctgcaggtaacttcgatgttaaggaggaaagatctgccgcttctttattgcagttgtccggtaatgccagtgatttttcatatggacggaactcaataactttgacagcatcagcataa

SEQ ID NO: 367 - DNA, 714 bp, synthetic construct
  misc_feature 1..714: note = Synthetic: mutant cI38 repressor nucleotide sequence
  source 1..714: mol_type = other DNA
tcagccaaacgtctcttcaggccactgactagcgataactttccccacaacggaacaactctcattgcatgggatcattgggtactgtgggtttagtggttgtaaaaacacctgaccgctatccctgatcagtttcttgaaggtaaactcaccacccccgagtctggctatgcagaaatcacctggctcaacagcctgctcagggtcaacgagaattaacattccgtcaggaaagcttggcttggagcctgttggtgctgtcatggaattaccttcaacctcaagccagaatgcagaatcactggcttttttggttgtgcttacccacctttccgcaccacctttggtaaaggttctaagctcaggtgagagcatccctgcctgaacatgagaaaaaacagggtactcatactcacttctaagtgacggctgcatactaaccgcttcatacatctcgtagatttctctggcgattgaagggctaaattcttcaacgctaactttgagaattcttgtaagcgatgcggcgttataagcatttaatgcattgatgccattaaataaggcaccaacgcctgactgccccatccccatcttgtctgcgacagattcctgggataagccaagttcatttttctttttttcataaattgctttaaggcgacgtgcgtcctcaagctgctcttgtgttaatggtttcttttttgtgctcat

SEQ ID NO: 368 - DNA, 798 bp, synthetic construct
  misc_feature 1..798: note = Synthetic: oligonucleotide
  source 1..798: mol_type = other DNA
tcagccaaacgtctcttcaggccactgactagcgataactttccccacaacggaacaactctcattgcatgggatcattgggtactgtgggtttagtggttgtaaaaacacctgaccgctatccctgatcagtttcttgaaggtaaactcaccacccccgagtctggctatgcagaaatcacctggctcaacagcctgctcagggtcaacgagaattaacattccgtcaggaaagcttggcttggagcctgttggtgctgtcatggaattaccttcaacctcaagccagaatgcagaatcactggcttttttggttgtgcttacccacctttccgcaccacctttggtaaaggttctaagctcaggtgagagcatccctgcctgaacatgagaaaaaacagggtactcatactcacttctaagtgacggctgcatactaaccgcttcatacatctcgtagatttctctggcgattgaagggctaaattcttcaacgctaactttgagaattcttgtaagcgatgcggcgttataagcatttaatgcattgatgccattaaataaggcaccaacgcctgactgccccatccccatcttgtctgcgacagattcctgggataagccaagttcatttttctttttttcataaattgctttaaggcgacgtgcgtcctcaagctgctcttgtgttaatggtttcttttttgtgctcatacgttaaatctatcaccgcaagggataaatatctaacaccgtgcgtgttgactattttacctctggcggtgataatggttgcat

SEQ ID NO: 369 - DNA, 22 bp, synthetic construct
  misc_feature 1..22: note = Synthetic: promoter
  source 1..22: mol_type = other DNA
ggaattgtgagcgctcacaatt

SEQ ID NO: 370 - DNA, 355 bp, synthetic construct
  misc_feature 1..355: note = Synthetic: OxyR binding sites
  source 1..355: mol_type = other DNA
tgtggcttttatgaaaatcacacagtgatcacaaattttaaacagagcacaaaatgctgcctcgaaatgagggcgggaaaataaggttatcagccttgttttctccctcattacttgaaggatatgaagctaaaacccttttttataaagcatttgtccgaattcggacataatcaaaaaagcttaattaagatcaatttgatctacatctctttaaccaacaatatgtaagatctcaactatcgcatccgtggattaattcaattataacttctctctaacgctgtgtatcgtaacggtaacactgtagaggggagcacattgatgcgaattcattaaagaggagaaaggtacc

SEQ ID NO: 371 - DNA, 228 bp, synthetic construct
  misc_feature 1..228: note = Synthetic: OxyR binding sites
  source 1..228: mol_type = other DNA
ttccgaaaattcctggcgagcagataaataagaattgttcttatcaatatatctaactcattgaatctttattagttttgtttttcacgcttgttaccactattagtgtgataggaacagccagaatagcggaacacatagccggtgctatacttaatctcgttaattactgggacataacatcaagaggatatgaaattcgaattcattaaagaggagaaaggtacc

SEQ ID NO: 372 - DNA, 334 bp, synthetic construct
  misc_feature 1..334: note = Synthetic: OxyR binding sites
  source 1..334: mol_type = other DNA
gcttagatcaggtgattgccctttgtttatgagggtgttgtaatccatgtcgttgttgcatttgtaagggcaacacctcagcctgcaggcaggcactgaagataccaaagggtagttcagattacacggtcacctggaaagggggccattttactttttatcgccgctggcggtgcaaagttcacaaagttgtcttacgaaggttgtaaggtaaaacttatcgatttgataatggaaacgcattagccgaatcggcaaaaattggttaccttacatctcatcgaaaacacggaggaagtatagatgcgaattcattaaagaggagaaaggtacc

SEQ ID NO: 373 - DNA, 134 bp, synthetic construct
  misc_feature 1..134: note = Synthetic: OxyR binding sites
  source 1..134: mol_type = other DNA
ctcgagttcattatccatcctccatcgccacgatagttcatggcgataggtagaatagcaatgaacgattatccctatcaagcattctgactgataattgctcacacgaattcattaaagaggagaaaggtacc

SEQ ID NO: 374 - DNA, 1921 bp, synthetic construct
  misc_feature 1..1921: note = Synthetic: propionate promoter
  source 1..1921: mol_type = other DNA
ttacccgtctggattttcagtacgcgcttttaaacgacgccacagcgtggtacggctgatccccaaataacgtgcggcggcgcgcttatcgccattaaagcgtgcgagcacctcctgcaatggaagcgcttctgctgacgagggcgtgatttctgctgtggtccccaccagttcaggtaataattgccgcataaattgtctgtccagtgttggtgcgggatcgacgcttaaaaaaagcgccaggcgttccatcatattccgcagttcgcgaatattaccgggccaatgatagttcagtagaagcggctgacactgcgtcagcccatgacgcaccgattcggtaaaagggatctccatcgcggccagcgattgttttaaaaagttttccgccagaggcagaatatcaggctgtcgctcgcgcaaggggggaagcggcagacgcagaatgctcaaacggtaaaacagatcggtacgaaaacgtccttgcgttatctcccgatccagatcgcaatgcgtggcgctgatcacccggacatctaccgggatcggctgatgcccgccaacgcgggtgacggctttttcctccagtacgcgtagaaggcgggtttgtaacggcagcggcatttcgccaatttcgtcaagaaacagcgtgccgccgtgggcgacctcaaacagccccgcacgtccacctcgtcttgagccggtaaacgctccctcctcatagccaaacagttcagcctccagcaacgactcggtaatcgcgccgcaattaacggcgacaaagggcggagaaggcttgttctgacggtggggctgacggttaaacaacgcctgatgaatcgcttgcgccgccagctctttcccggtccctgtttccccctgaatcagcactgccgcgcgggaacgggcatagagtgtaatcgtatggcgaacctgctccatttgtggtgaatcgccgaggatatcgctcagcgcataacgggtctgtaatcccttgctggaggtatgctggctatactgacgccgtgtcaggcgggtcatatccagcgcatcatggaaagcctgacgtacggtggccgctgaataaataaagatggcggtcattcctgcctcttccgccaggtcggtaattagtcctgccccaattacagcctcaatgccgttagctttgagctcgttaatttgcccgcgagcatcctcttcagtgatatagcttcgctgttcaagacggaggtgaaacgttttctgaaaggcgaccagagccggaatggtctcctgataggtcacgattcccattgaggaagtcagctttcccgcttttgccagagcctgtaatacatcgaatccgctgggtttgatgaggatgacaggtaccgacagtcggctttttaaataagcgccgttggaacctgccgcgataatcgcgtcgcagcgttcggttgccagttttttgcgaatgtaggctactgccttttcaaaaccgagctgaataggcgtgatcgtcgccagatgatcaaactccaggctgatatcccgaaatagttcgaacaggcgcgttaccgagaccgtccagatcaccggtttatcgctattatcgcgcgaagcgctatgcacagtaaccatcgtcgtagattcatgtttaaggaacgaattcttgttttatagatgtttcgttaatgttgcaatgaaacacaggcctccgtttcatgaaacgttagctgactcgtttttcttgtgactcgtctgtcagtattaaaaaagatttttcatttaactgattgtttttaaattgaattttatttaatggtttctcggtttttgggtctggcatatcccttgctttaatgagtgcatcttaattaacaattcaataacaagagggctgaatagtaatttcaacaaaataacgagcattcgaatg

SEQ ID NO: 375 - DNA, 305 bp, synthetic construct
  misc_feature 1..305: note = Synthetic: oligonucleotide
  source 1..305: mol_type = other DNA
cagacattgccgtcactgcgtcttttactggctcttctcgctaacccaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatccagcctgacgctttttttcgcaactctctactgtttctccatacctctagaaataattttgtttaactttaagaaggagatatacat

SEQ ID NO: 376 - DNA, 897 bp, synthetic construct
  misc_feature 1..897: note = Synthetic: oligonucleotide
  source 1..897: mol_type = other DNA
ttattcacaacctgccctaaactcgctcggactcgccccggtgcattttttaaatactcgcgagaaatagagttgatcgtcaaaaccgacattgcgaccgacggtggcgataggcatccgggtggtgctcaaaagcagcttcgcctgactgatgcgctggtcctcgcgccagcttaatacgctaatccctaactgctggcggaacaaatgcgacagacgcgacggcgacaggcagacatgctgtgcgacgctggcgatatcaaaattactgtctgccaggtgatcgctgatgtactgacaagcctcgcgtacccgattatccatcggtggatggagcgactcgttaatcgcttccatgcgccgcagtaacaattgctcaagcagatttatcgccagcaattccgaatagcgcccttccccttgtccggcattaatgatttgcccaaacaggtcgctgaaatgcggctggtgcgcttcatccgggcgaaagaaaccggtattggcaaatatcgacggccagttaagccattcatgccagtaggcgcgcggacgaaagtaaacccactggtgataccattcgtgagcctccggatgacgaccgtagtgatgaatctctccaggcgggaacagcaaaatatcacccggtcggcagacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcgttaaacgagtatcccggcagcaggggatcattttgcgcttcagccatacttttcatactcccgccattcagagaagaaaccaattgtccatattgcat

SEQ ID NO: 377 - protein, 298 aa, synthetic construct
  REGION 1..298: note = Synthetic: polypeptide
MQYGQLVSSLNGGSMKSMAEAQNDPLLPGYSFNAHLVAGLTPIEANGYLDFFIDRPLGMKGYILNLTIRGQGVVKNQGREFVCRPGDILLFPPGEIHHYGRHPEAHEWYHQWVYFRPRAYWHEWLNWPSIFANTGFFRPDEAHQPHFSDLFGQIINAGQGEGRYSELLAINLLEQLLLRRMEAINESLHPPMDNRVREACQYISDHLADSNFDIASVAQHVCLSPSRLSHLFRQQLGISVLSWREDQRISQAKLLLSTTRMPIATVGRNVGFDDQLYFSRVFKKCTGASPSEFRAGCE

SEQ ID NO: 378 - DNA, 280 bp, synthetic construct
  misc_feature 1..280: note = Synthetic: oligonucleotide
  source 1..280: mol_type = other DNA
cggtgagcatcacatcaccacaattcagcaaattgtgaacatcatcacgttcatctttccctggttgccaatggcccattttcctgtcagtaacgagaaggtcgcgaatcaggcgctttttagactggtcgtaatgaaattcagctgtcaccggatgtgctttccggtctgatgagtccgtgaggacgaaacagcctctacaaataattttgtttaaaacaacacccactaagataactctagaaataattttgtttaactttaagaaggagatatacat

SEQ ID NO: 379 - DNA, 48 bp, synthetic construct
  misc_feature 1..48: note = Synthetic: PlacI (promoter for lacI in reverse orientation)
  source 1..48: mol_type = other DNA
ccgggcgctatcatgccataccgcgaaaggttttgcgccattcgatgg

SEQ ID NO: 380 - DNA, 1157 bp, synthetic construct
  misc_feature 1..1157: note = Synthetic: PlacI-RBS - lacI (reverse orientation)
  source 1..1157: mol_type = other DNA
tcactgcccgctttccagtcgggaaacctgtcgtgccagctgcattaatgaatcggccaacgcgcggggagaggcggtttgcgtattgggcgccagggtggtttttcttttcaccagtgagactggcaacagctgattgcccttcaccgcctggccctgagagagttgcagcaagcggtccacgctggtttgccccagcaggcgaaaatcctgtttgatggtggttaacggcgggatataacatgagctatcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcctcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcatattcaccaccctgaattgactctcttccgggcgctatcatgccataccgcgaaaggttttgcgccattcgatgg

SEQ ID NO: 381 - DNA, 29 bp, synthetic construct
  misc_feature 1..29: note = Synthetic: Ptac (minimal promoter for gene expression includes -10 and -35 region)
  source 1..29: mol_type = other DNA
ttgacaattaatcatcggctcgtataatg

SEQ ID NO: 382 - DNA, 60 bp, synthetic construct
  misc_feature 1..60: note = Synthetic: Exemplary spacer region
  source 1..60: mol_type = other DNA
cgcgccgcttcgtcaggccacatagctttcttgttctgatcggaacgatcgttggctgtg

SEQ ID NO: 383 - DNA, 60 bp, synthetic construct
  misc_feature 1..60: note = Synthetic: Exemplary pTac promoter comprising -10 and -35 regions and Lac operon
  source 1..60: mol_type = other DNA
ttgacaattaatcatcggctcgtataatgtgtggaattgtgagcgctcacaattagctgt

SEQ ID NO: 384 - DNA, 26 bp, synthetic construct
  misc_feature 1..26: note = Synthetic: Exemplary LacI RBS (reverse orientation)
  source 1..26: mol_type = other DNA
attcaccaccctgaattgactctctt

SEQ ID NO: 385 - DNA, 17 bp, synthetic construct
  misc_feature 1..17: note = Synthetic: operator
  source 1..17: mol_type = other DNA
tatcaccgcaagggata

SEQ ID NO: 386 - DNA, 642 bp, synthetic construct
  misc_feature 1..642: note = Synthetic: oligonucleotide
  source 1..642: mol_type = other DNA
ttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaattttt

SEQ ID NO: 387 - DNA, 225 bp, synthetic construct
  misc_feature 1..225: note = Synthetic: oligonucleotide
  source 1..225: mol_type = other DNA
tcacctttcccggattaaacgcttttttgcccggtggcatggtgctaccggcgatcacaaacggttaattatgacacaaattgacctgaatgaatatacagtattggaatgcattacccggagtgttgtgtaacaatgtctggccaggtttgtttcccggaaccgaggtcacaacatagtaaaagcgctattggtaatggtacaatcgcgcgtttacacttattc

SEQ ID NO: 388 - DNA, 213 bp, synthetic construct
  misc_feature 1..213: note = Synthetic: PfNRS
  source 1..213: mol_type = other DNA
ggtaccagttgttcttattggtggtgttgctttatggttgcatcgtagtaaatggttgtaacaaaagcaatttttccggctgtctgtatacaaaaacgccgcaaagtttgagcgaagtcaataaactctctacccattcagggcaatatctctcttggatccaaagtgaactctagaaataattttgtttaactttaagaaggagatatacat

SEQ ID NO: 389 - DNA, 43 bp, synthetic construct
  misc_feature 1..43: note = Synthetic: Ribosome binding site and leader region
  source 1..43: mol_type = other DNA
ctctagaaataattttgtttaactttaagaaggagatatacat

SEQ ID NO: 390 - DNA, 879 bp, synthetic construct
  misc_feature 1..879: note = Synthetic: dapA dna
  source 1..879: mol_type = other DNA
atgttcacgggaagtattgtcgcgattgttactccgatggatgaaaaaggtaatgtctgtcgggctagcttgaaaaaactgattgattatcatgtcgccagcggtacttcggcgatcgtttctgttggcaccactggcgagtccgctaccttaaatcatgacgaacatgctgatgtggtgatgatgacgctggatctggctgatgggcgcattccggtaattgccgggaccggcgctaacgctactgcggaagccattagcctgacgcagcgcttcaatgacagtggtatcgtcggctgcctgacggtaaccccttactacaatcgtccgtcgcaagaaggtttgtatcagcatttcaaagccatcgctgagcatactgacctgccgcaaattctgtataatgtgccgtcccgtactggctgcgatctgctcccggaaacggtgggccgtctggcgaaagtaaaaaatattatcggaatcaaagaggcaacagggaacttaacgcgtgtaaaccagatcaaagagctggtttcagatgattttgttctgctgagcggcgatgatgcgagcgcgctggacttcatgcaattgggcggtcatggggttatttccgttacggctaacgtcgcagcgcgtgatatggcccagatgtgcaaactggcagcagaagggcattttgccgaggcacgcgttattaatcagcgtctgatgccattacacaacaaactatttgtcgaacccaatccaatcccggtgaaatgggcatgtaaggaactgggtcttgtggcgaccgatacgctgcgcctgccaatgacaccaatcaccgacagtggccgtgagacggtcagagcggcgcttaaacatgccggtttgctgtaa

SEQ ID NO: 391 - DNA, 795 bp, synthetic construct
  misc_feature 1..795: note = Synthetic: ThyA dna
  source 1..795: mol_type = other DNA
atgaaacagtatttagaactgatgcaaaaagtgctcgacgaaggcacacagaaaaacgaccgtaccggaaccggaacgctttccatttttggtcatcagatgcgttttaacctgcaagatggattcccgctggtgacaactaaacgttgccacctgcgttccatcatccatgaactgctgtggtttcttcagggcgacactaacattgcttatctacacgaaaacaatgtcaccatctgggacgaatgggccgatgaaaacggcgacctcgggccagtgtatggtaaacagtggcgtgcctggccaacgccagatggtcgtcatattgaccagatcactacggtactgaaccagctgaaaaacgacccggattcgcgccgcattattgtttcagcgtggaacgtaggcgaactggataaaatggcgctggcaccgtgccatgcattcttccagttctatgtggcagacggcaaactctcttgccagctttatcagcgctcctgtgacgtcttcctcggcctgccgttcaacattgccagctacgcgttattggtgcatatgatggcgcagcagtgcgatctggaagtgggtgattttgtctggaccggtggcgacacgcatctgtacagcaaccatatggatcaaactcatctgcaattaagccgcgaaccgcgtccgctgccgaagttgattatcaaacgtaaacccgaatccatcttcgactaccgtttcgaagactttgagattgaaggctacgatccgcatccgggcattaaagcgccggtggctatctaa

SEQ ID NO: 392 - protein, 292 aa, synthetic construct
  REGION 1..292: note = Synthetic: dapA protein
MFTGSIVAIVTPMDEKGNVCRASLKKLIDYHVASGTSAIVSVGTTGESATLNHDEHADVVMMTLDLADGRIPVIAGTGANATAEAISLTQRFNDSGIVGCLTVTPYYNRPSQEGLYQHFKAIAEHTDLPQILYNVPSRTGCDLLPETVGRLAKVKNIIGIKEATGNLTRVNQIKELVSDDFVLLSGDDASALDFMQLGGHGVISVTANVAARDMAQMCKLAAEGHFAEARVINQRLMPLHNKLFVEPNPIPVKWACKELGLVATDTLRLPMTPITDSGRETVRAALKHAGLL

SEQ ID NO: 393 - protein, 264 aa, synthetic construct
  REGION 1..264: note = Synthetic: ThyA protein
MKQYLELMQKVLDEGTQKNDRTGTGTLSIFGHQMRFNLQDGFPLVTTKRCHLRSIIHELLWFLQGDTNIAYLHENNVTIWDEWADENGDLGPVYGKQWRAWPTPDGRHIDQITTVLNQLKNDPDSRRIIVSAWNVGELDKMALAPCHAFFQFYVADGKLSCQLYQRSCDVFLGLPFNIASYALLVHMMAQQCDLEVGDFVWTGGDTHLYSNHMDQTHLQLSREPRPLPKLIIKRKPESIFDYRFEDFEIEGYDPHPGIKAPVAI

SEQ ID NO: 394 - DNA, 2124 bp, synthetic construct
  misc_feature 1..2124: note = Synthetic: HlyB coding sequence
  source 1..2124: mol_type = other DNA
atggattcttgtcataaaattgattatgggttatacgccctggagattttagcccaataccataacgtctctgttaacccggaagaaattaaacatagatttgacacagacgggactggtctgggattaacgtcatggttgcttgctgcgaaatctttagaactaaaggtaaaacaggtaaaaaaaacaattgaccgattaaactttatttctttgcccgcattagtctggagagaggatggacgtcattttattctgactaaagtcagtaaagaagcaaacagatatcttatttttgatctggagcaacgaaatccccgtgttctcgaacagtctgagtttgaggcgttatatcaggggcatattattcttattgcttcccgttcttctgttaccgggaaactggcaaaatttgactttacctggtttatccctgccattataaaatacagaaaaatatttattgaaacccttgttgtatctgtttttttacaattatttgcattaataaccccccttttttttcaggtggttatggacaaagtattagtacacagggggttttcaacccttaatgttattactgtcgcattatctgttgtggtggtgtttgagattatactcagcggtttaagaacttacatttttgcacatagtacaagtcggattgatgttgagttgggtgccaaactcttccggcatttactggcgctaccgatctcttattttgagagtcgtcgtgttggtgatactgttgccagggtaagagaattagaccagatccgtaattttctgacaggacaggcattaacatctgttctggacttattattttcattcatattttttgcggtaatgtggtattacagcccaaagcttactctggtgatcttattttcgctgccctgttatgctgcatggtctgtttttattagccccattttgcgacgtcgccttgatgataagttttcacggaatgcggataatcaatctttcctggtggaatcagtcacggcgattaacactataaaagctatggcagtctcacctcagatgacgaacatatgggacaaacaattggcaggatatgttgctgcaggctttaaagtgacagtattagccaccattggtcaacaaggaatacagttaatacaaaagactgttatgatcatcaacctgtggttgggagcacacctggttatttccggggatttaagtattggtcagttaattgcttttaatatgcttgctggtcagattgttgcaccggttattcgccttgcacaaatctggcaggatttccagcaggttggtatatcagttacccgccttggtgatgtgcttaactctccaactgaaagttatcatgggaaactggcattaccggaaattaatggtaatatcacttttcgtaatatccggtttcgctataagcctgactctccggttattttagataatatcaatctcagtattaagcagggggaggttattggtattgtcggacgttctggttcaggaaaaagcacattaactaaattaattcaacgtttttatattcctgaaaatggccaggtcttaattgatggacatgatcttgcgttggccgatcctaactggttacgtcgtcaggtgggggttgtgttgcaggacaatgtgctgcttaatcgcagtattattgataatatctcactggctaatcctggtatgtccgtcgaaaaagttatttatgcagcgaaattagcaggcgctcatgattttatttctgaattgcgtgaggggtataacaccattgtcggggaacagggggcaggattatccggaggtcaacgtcaacgcatcgcaattgcaagggcgctggtgaacaaccctaaaatacttatttttgatgaagcaaccagtgctctggattatgagtcggagcatatcatcatgcgcaatatgcacaaaatatgtaagggcagaacggttataatcattgctcatcgtctgtctacagtaaaaaatgcagaccgcattattgtcatggaaaaagggaaaattgttgaacagggtaaacataaggaactgctttctgaaccggaaagtttatacagttacttatatcagttacagtcagactaa

SEQ ID NO: 395 - DNA, 1437 bp, synthetic construct
  misc_feature 1..1437: note = Synthetic: HlyD coding sequence
  source 1..1437: mol_type = other DNA
atgaaaacatggttaatggggttcagcgagttcctgttgcgctataaacttgtctggagtgaaacatggaaaatccggaagcaattagatactccggtacgtgaaaaggacgaaaatgaattcttacccgctcatctggaattaattgaaacgccggtatccagacggccgcgtctggttgcttattttattatggggtttctggttattgctgtcattttatctgttttaggtcaggtggaaattgttgccactgcaaatgggaaattaacactaagtgggcgcagcaaagaaattaaacctattgaaaactcaatagttaaagaaattatcgtaaaagaaggagagtcagtccggaaaggggatgtgttattaaagcttacagcactgggagctgaagctgatacgttaaaaacacagtcatcactgttacagaccaggctggaacaaactcggtatcaaattctgagcaggtcaattgaattaaataaactacctgaactgaagcttcctgatgagccttattttcagaatgtatctgaagaggaagtactgcgtttaacttctttgataaaagaacagttttccacatggcaaaatcagaagtatcaaaaagaactgaatctggataagaaaagagcagagcgattaacaatacttgcccgtataaaccgttatgaaaatttatcgagagttgaaaaaagccgtctggatgatttcaggagtttattgcataaacaggcaattgcaaaacatgctgtacttgagcaggagaataaatatgtcgaggcagcaaatgaattacgggtttataaatcgcaactggagcaaattgagagtgagatattgtctgcaaaagaagaatatcagcttgtcacgcagctttttaaaaatgaaattttagacaagctaagacaaacaacagacaacattgagttattaactctggagttagagaaaaatgaagagcgtcaacaggcttcagtaatcagggcccctgtttcgggaaaagttcagcaactgaaggttcatactgaaggtggggttgttacaacagcggaaacactgatggtcatcgttccggaagatgacacgctggaggttactgctctggtacaaaataaagatattggttttattaacgtcgggcagaatgccatcattaaagtggaggcctttccttacacccgatatggttatctggtgggtaaggtgaaaaatataaatttagatgcaatagaagaccagaaactgggactcgtttttaatgtcattgtttctgttgaagagaatgatttgtcaaccgggaataagcacattccattaagctcgggtatggctgtcactgcagaaataaagactggaatgcgaagcgtaatcagctatcttcttagtcctctggaagagtctgtaacagaaagtttacatgagcgttaa

SEQ ID NO: 396 - protein, 707 aa, synthetic construct
  REGION 1..707: note = Synthetic: HlyB
MDSCHKIDYGLYALEILAQYHNVSVNPEEIKHRFDTDGTGLGLTSWLLAAKSLELKVKQVKKTIDRLNFISLPALVWREDGRHFILTKVSKEANRYLIFDLEQRNPRVLEQSEFEALYQGHIILIASRSSVTGKLAKFDFTWFIPAIIKYRKIFIETLVVSVFLQLFALITPLFFQVVMDKVLVHRGFSTLNVITVALSVVVVFEIILSGLRTYIFAHSTSRIDVELGAKLFRHLLALPISYFESRRVGDTVARVRELDQIRNFLTGQALTSVLDLLFSFIFFAVMWYYSPKLTLVILFSLPCYAAWSVFISPILRRRLDDKFSRNADNQSFLVESVTAINTIKAMAVSPQMTNIWDKQLAGYVAAGFKVTVLATIGQQGIQLIQKTVMIINLWLGAHLVISGDLSIGQLIAFNMLAGQIVAPVIRLAQIWQDFQQVGISVTRLGDVLNSPTESYHGKLALPEINGNITFRNIRFRYKPDSPVILDNINLSIKQGEVIGIVGRSGSGKSTLTKLIQRFYIPENGQVLIDGHDLALADPNWLRRQVGVVLQDNVLLNRSIIDNISLANPGMSVEKVIYAAKLAGAHDFISELREGYNTIVGEQGAGLSGGQRQRIAIARALVNNPKILIFDEATSALDYESEHIIMRNMHKICKGRTVIIIAHRLSTVKNADRIIVMEKGKIVEQGKHKELLSEPESLYSYLYQLQSD

SEQ ID NO: 397 - protein, 478 aa, synthetic construct
  REGION 1..478: note = Synthetic: HlyD
MKTWLMGFSEFLLRYKLVWSETWKIRKQLDTPVREKDENEFLPAHLELIETPVSRRPRLVAYFIMGFLVIAVILSVLGQVEIVATANGKLTLSGRSKEIKPIENSIVKEIIVKEGESVRKGDVLLKLTALGAEADTLKTQSSLLQTRLEQTRYQILSRSIELNKLPELKLPDEPYFQNVSEEEVLRLTSLIKEQFSTWQNQKYQKELNLDKKRAERLTILARINRYENLSRVEKSRLDDFRSLLHKQAIAKHAVLEQENKYVEAANELRVYKSQLEQIESEILSAKEEYQLVTQLFKNEILDKLRQTTDNIELLTLELEKNEERQQASVIRAPVSGKVQQLKVHTEGGVVTTAETLMVIVPEDDTLEVTALVQNKDIGFINVGQNAIIKVEAFPYTRYGYLVGKVKNINLDAIEDQKLGLVFNVIVSVEENDLSTGNKHIPLSSGMAVTAEIKTGMRSVISYLLSPLEESVTESLHER

SEQ ID NO: 398 - DNA, 106 bp, synthetic construct
  misc_feature 1..106: note = Synthetic: RBS-phoA
  source 1..106: mol_type = other DNA
ctctagaaataattttgtttaactttaagaaggagatatacatatgaaacaaagcactattgcactggcactcttaccgttactgtttacccctgtgacaaaagcg

SEQ ID NO: 399 - DNA, 109 bp, synthetic construct
  misc_feature 1..109: note = Synthetic: RBS-ompF
  source 1..109: mol_type = other DNA
ctctagaaataattttgtttaactttaagaaggagatatacatatgatgaagcgcaatattctggcagtgatcgtccctgctctgttagtagcaggtactgcaaacgct

SEQ ID NO: 400 - DNA, 66 bp, synthetic construct
  misc_feature 1..66: note = Synthetic: ompF
  source 1..66: mol_type = other DNA
atgatgaagcgcaatattctggcagtgatcgtccctgctctgttagtagcaggtactgcaaacgct

SEQ ID NO: 401 - DNA, 88 bp, synthetic construct
  misc_feature 1..88: note = Synthetic: RBS-cvaC
  source 1..88: mol_type = other DNA
ctctagaaataattttgtttaactttaagaaggagatatacatatgagaactctgactctaaatgaattagattctgtttctggtggt

SEQ ID NO: 402 - DNA, 45 bp, synthetic construct
  misc_feature 1..45: note = Synthetic: cvaC
  source 1..45: mol_type = other DNA
atgagaactctgactctaaatgaattagattctgtttctggtggt

SEQ ID NO: 403 - DNA, 88 bp, synthetic construct
  misc_feature 1..88: note = Synthetic: RBS-phoA (Optimized)
  source 1..88: mol_type = other DNA
gacgccagagagttaagggggttaaatgaaacaatcgaccatcgcattggcgctgcttcctctattgttcacaccggtgacaaaggca

SEQ ID NO: 404 - DNA, 63 bp, synthetic construct
  misc_feature 1..63: note = Synthetic: optimized phoA
  source 1..63: mol_type = other DNA
atgaaacaatcgaccatcgcattggcgctgcttcctctattgttcacaccggtgacaaaggca

SEQ ID NO: 405 - DNA, 160 bp, synthetic construct
  misc_feature 1..160: note = Synthetic: RBS-TorA
  source 1..160: mol_type = other DNA
ctctagaaataattttgtttaactttaagaaggagatatacatatgaacaataacgatctctttcaggcatcacgtcggcgttttctggcacaactcggcggcttaaccgtcgccgggatgctggggccgtcattgttaacgccgcgacgtgcgactgcg

SEQ ID NO: 406 - DNA, 117 bp, synthetic construct
  misc_feature 1..117: note = Synthetic: TorA
  source 1..117: mol_type = other DNA
atgaacaataacgatctctttcaggcatcacgtcggcgttttctggcacaactcggcggcttaaccgtcgccgggatgctggggccgtcattgttaacgccgcgacgtgcgactgcg

SEQ ID NO: 407 - DNA, 148 bp, synthetic construct
  misc_feature 1..148: note = Synthetic: RBS-TorA alternate
  source 1..148: mol_type = other DNA
cccacattcgaggtactaaatgaacaataacgatctctttcaggcatcacgtcggcgttttctggcacaactcggcggcttaaccgtcgccgggatgctggggacgtcattgttaacgccgcgccgtgcgactgcggcgcaagcggcg

SEQ ID NO: 408 - DNA, 129 bp, synthetic construct
  misc_feature 1..129: note = Synthetic: TorA (alternate)
  source 1..129: mol_type = other DNA
atgaacaataacgatctctttcaggcatcacgtcggcgttttctggcacaactcggcggcttaaccgtcgccgggatgctggggacgtcattgttaacgccgcgccgtgcgactgcggcgcaagcggcg

SEQ ID NO: 409 - DNA, 130 bp, synthetic construct
  misc_feature 1..130: note = Synthetic: RBS-fdnG
  source 1..130: mol_type = other DNA
accctattacacacctaaggaggccaaatacatggacgtcagtcgcagacaattttttaaaatctgcgcgggcggtatggcgggaacaacagtagcagcattgggctttgccccgaagcaagcactggct

SEQ ID NO: 410 - DNA, 99 bp, synthetic construct
  misc_feature 1..99: note = Synthetic: fdnG
  source 1..99: mol_type = other DNA
atggacgtcagtcgcagacaattttttaaaatctgcgcgggcggtatggcgggaacaacagtagcagcattgggctttgccccgaagcaagcactggct

SEQ ID NO: 411 - DNA, 168 bp, synthetic construct
  misc_feature 1..168: note = Synthetic: RBS-dmsA
  source 1..168: mol_type = other DNA
tacgcaaaaaacataatttaagagaggataaacatgaaaacgaaaatccctgatgcggtattggctgctgaggtgagtcgccgtggtttggtaaaaacgacagcgatcggcggcctggcaatggccagcagcgcattaacattaccttttagtcggattgcgcacgct

SEQ ID NO: 412 - DNA, 135 bp, synthetic construct
  misc_feature 1..135: note = Synthetic: dmsA
  source 1..135: mol_type = other DNA
atgaaaacgaaaatccctgatgcggtattggctgctgaggtgagtcgccgtggtttggtaaaaacgacagcgatcggcggcctggcaatggccagcagcgcattaacattaccttttagtcggattgcgcacgct

SEQ ID NO: 413 - DNA, 712 bp, synthetic construct
  misc_feature 1..712: note = Synthetic: TetR/TetA Promoter
  source 1..712: mol_type = other DNA
gaattcgttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttattttaccactccctatcagtgatagagaaaagtgaa

SEQ ID NO: 414 - DNA, 49496 bp, synthetic construct
  misc_feature 1..49496: note = Synthetic: Phage 3 modification
  source 1..49496: mol_type = other DNA
aggcctctcctcgcgagaggcattttttatttgatgggataaagatctttgcgcttatacggttggatttcgcccggtttgcgagttttcagcaattttaatatccaggtgtattgttctggtcgcggaccaacaaaaatctcgacttcttcattcatccgccgcgcaatcgtatgatcatccgcctctaacagatcatccatcggtgggcgcacctgaatcgtcagacgatgcgtcttgccatcataaatcggaaatagcggtacaacgcgcgcacggcacactttcatcaaacgaccaatcgcgggcaacgtcgctttataggtggcaaagaaatcaacaaattcgctgtgttctgggccatgatcctgatcgggtaaataatatccccagtaaccctgacgtaccgactggatgaatggtttaataccatcatttctcgcatgcagacgaccaccaaagcgacggcgcaccgtgttccagacataatcaaaaaccgggttgccctgattatggaacatcgctgccattttctgcccttgcgaggccatcagcatggcaggaatatcgacggcccaaccgtgcggcaccagaaaaatcactttctcgttattacgtcgtatctcttcgatgatctccagcccttgccagtcaacgcgcggctgaattttctccggcccgcgtattgccaactcagccatcattaccatcgcttgcggcgcggtggcaaacatctcatctacaatcgcttcgcgttcagcttcactacgttctggaaagcagagcgacagattgattaacgcacgacggcgtgagctttttcccagtcgtccggcaaaacgtcccagccgtgccagaatgggatcacggaactttggcggcgttaaagcgatacccgccatcgctgctacgcccagccatgctccccagtagcgcgggtggcgaaaggatttatcaaactcaggaatgtattcgctattattttttttcgtttccatgcttttccagtttcggataaggcaaaaatcaatctggtgatagtgtagcggcgcaacttgccccgcaccaaataaaaaagccggtactgactgcgtaccggctgcgaatggatgttaattaatcaaaccgtagctgcggcacaatctctttggcctgtgccaggaattcgcgacgatcggagccggtcagcccttcggtacgcggcagttttgccgtcagcgggtttacggcctgctggtttatccatacttcatagtgcagatgcggcccggttgaacgtccggtattaccggaaagcgcgatacggtcgccacgtttcaccttctgtcccggtttcaccaggatcttgcgcaagtgcatataacgcgtggtgtagctgcgaccatgacgaatagccacataataacctgctgcgccactacgtttggcaaccaccacttcaccgtcacccactgaaagcactggcgtaccttgtggcatggcaaaatcaacacctctgtgtggcgcaacgcgaccggtcaccggattagtacgacgcgggttaaagttagatgagatacggaactgtttcgccgtcgggaatcgcaagaatcctttcgccagaccagtaccgttacgatcgtagaatttgccatcttcagcgcggattgcgtaataatctttaccttctgaacgcaaacgtacgcccagcagctggctttgctcacgtttaccatcaagcatttctcgtgacattaacaccgcaaattcatcgccttttttcagtttgcggaaatccatttgccactgcatggctttaatcactgcgctcacttcggcgctggttaaaccggcgtttctggcgctggcaacaaagcttcccccgacggtacctttcagcagattgttgacccactctccttgctgcatttcgctggtcattttaaaaccgttagcggcagtacggtcataggttcgggtttcacgacgagacacttcccaggtgaggcgctgcagttcgccgtccgcggttaatgtccaggagagttgttgaccgattttcaggttacgcaattctttgtcggcagcagccagttgggtgatatcacccatatcaataccatactgattgagaatgctgcttagcgtatcgccagtggaaacaacatattcatgcacgcccgcttcaccggcgattttgtcatccagttcgtcctggggaatggcttcatcttcttgtgcagcttgatcaatcggctcactggcttcaggtaagagcgaacgaatttcgttctgttccagctcaatggttttgacaattggcgtggcatcgcggtgataaacatagggccgccagacagcgacggccagagtaagaacggtgagcgaccccaacataacgcggtgtggtcgcggtaaattattaaacgccagggcgacagagcgggctatctgttgcacgtaatcacttcctcattaatctcctttcaggcagctcgcatactggttggctaattgattcaggaattctgaatagcttgttttacccagtttgatattcgtccccaggggatccaacgttcccatacgaacggatgtccctcgtgcgacgctctcaacgaccgctggcctgaactgtggctcagcaaaaacgcaggttgctttttgctcaaccaactgtgttcttatttcatgtaaacgctgcgcgccaggttgaatctcagggttaacggtaaaatgaccaagcggtgtcagtccgaactgtttttcgaaatagccgtaagcatcgtgaaaaacgaaataacctttccccttgagcggcgcgagctcgttaccaacctgcttttcggttgaggctaattgtgcctcaaaatccttcaggttggcgtcaagtttggctcgactttgcggcataagttccactaattttccatggattgcaaccgctgtagcccgcgctatctctggggaaagccaaagatgcatgttgaaatcgccgtgatggtgatcttcgtcacttttttccgcgtggtcgtgatcatcatcatcgccgtgaatacttttcatcagcagcggtttcacattctctagctgcgcaatcgttacctgtttcgcttcaggtaatttacttaccggtttttgcatgaacgcttccatctccgggccaacccaaacgactaagtccgcgttctgtaagcgttttacatctgatggacgcagtgaataatcatgttctgaagccccgtcaggtagtaaaacctccgtttctgttaccccatcagcaatggcagaagcgatgaacccaacgggtttaagcgaagcgacaacggcagcatctgcggcctgtgttgcaccgccccagagagcggcggataatgctgcgaaaagaagcgtttttttatgtaacataatgcgaccaatcatcgtaatgaatatgagaagtgtgatattataacatttcatgactactgcaagactaaaattaacatgacaagtctggtttccctggaaaatgtctcggtttcttttggccaacgccgcgtcctctctgatgtgtcgctggaacttaaacctggaaaaattttgactttacttgggccaaacggcgcaggtaagtcgacactggtacgggtagtgctcgggctggtaacacccgatgaaggggttatcaagcgcaacggaaaactgcgcatcggctatgtaccgcagaagctgtatctcgacaccacgttgccactgaccgtaaaccgttttttacgcttacgccctggcacacataaagaagatattttgcctgcactgaaacgtgtccaggccgggcatctgattaacgcaccgatgcaaaagctctcgggtggcgaaacgcagcgtgtactgttagcgcgagcattgttaaatcgaccgcaattattagtgctggatgaacccactcagggcgtggatgtgaatggtcaggtggcgttatatgaccttattgaccaactgcgtcgcgaactggattgtggcgttttaatggtatctcacgatctgcatctggtaatggcaaaaaccgatgaagtgctttgcctgaatcaccacatttgttgttccggcacaccggaagttgtttccctgcatccggagtttatttctatgtttggtcctcgtggtgctgaacaactgggtatctatcgccatcatcataatcatcgtcacgatttacagggacgaattgttttgcgtcggggaaatgatcgctcatgattgaattattatttcccggttggttagccgggatcatgctcgcctgtgccgcgggtccgctgggttcgtttgtagtctggcgtcgtatgtcttatttcggtgatacgctggctcatgcctcattacttggcgtcgcgtttggtttgttgctggacgtgaatccattctatgcggtgattgccgttacgctgctgctggcgggcggtctggtatggctggagaagcgtccacagctggcgatcgacacgttattagggattatggcgcacagtgccctgtcgctgggcctggtggtcgttagtctgatgtctaatattcgtgttgatttgatggcttacctgttcggtgatttactggcagtgacgccagaagatctcatctctattgcgattggcgtggtcatcgtggtggctattttgttctggcaatggcgcaatttgctgtcgatgacgattagcccggatctggcgtttgttgatggtgtgaaattacagcgcgtgaaattgttgttgatgctggtgacggcattgacgattggtgtagcgatgaaattcgtcggcgcgttgattattacttcactgctgattattcctgctgctactgcacgtcgctttgcccgcacgccggaacagatggctggtgtcgctgttttggtggggatggtggcagtgactggcggtttaaccttttccgcattttacgatacacctgcaggcccgtcggtggtgctatgcgcggcactgttatttattatcagtatgatgaaaaagcaggccagctaatctgtcgctgaacacatttgtcggatgcggcgcgagcgccttatcccacctgcggttcgctatctctggtaggcctgataagacgcgaacagcgtcgcatcaggcacactgccagtgtcggatgcggctcgagcgaccaatccgacttacggcatttctggcggcgtgatgccgaagtggttccacgcccgcactgtcgccatacgcccgcgcggtgtacgctgcaaaaagccttgctgaatcaaataaggttccagtacatcctcaatggtttcacgttcttcgccaatggctgccgccaggttatccagacctaccggcccaccaaagaacttatcgattaccgccagcaacaatttgcggtccatataatcgaaaccttcagcatcgacattcaacatatccagcgcctgagcagcgatatctgccgagatggtgccatcgtgcttcacttcagcgaaatcacgcactcgacgcagcagacggttggcaatacgtggcgtaccgcgcgcacgacgagcaacttccagcgcgccgtcatcactcatctcaagccccataaagcgtgcgctgcgactgacgatatattgcagatccggcacctgataaaactccagacgttgcacaataccaaaacgatcgcgcaacggtgatgtcagcgaacctgcgcgcgtggttgcaccaatcagggtaaacggcggcaaatcaattttaatggagcgtgccgccggaccttcaccaatcatgatatccagttggtaatcttccattgccggatacaacacctcttccaccactggtgaaagacggtggatctcatcaataaacagtacatcgtgtggttcaaggttagtgagcattgctgccagatcgcccgccttttccagcaccggaccagaagtcgtgcgtaaattaacgcccatttcattggcgacaatattggcaagcgtagttttacccaaccccggaggaccaaaaatcaatagatgatcgagggcatcgccgcgcagtttcgctgctttgatgaaaatctccatctgcgaacgaacctgcggctgaccaacatactcttccagtaatttagggcgaatggcgcgatctgccacatcttccggcaaagtggtaccggcagaaatcagacggtctgcttcaatcatcctttacctcataacgcggcgcgtagggcttcgcgaattaatgtttcactgctggcgtcagggcgagcgattttgctcaccatgcggcttgcttcttgtggtttatagcccagtgccaccagcgcagcaaccgcttcctgttcagcatcgtcggtcgccgggctggcaggagacgtgagtaccaggtcggcggctggcgtaaagagatcgccatgcaaacctttaaatcggtctttcatttcgacaatcaagcgttcggcggtttttttgccaatacccggcagtttcaccagtgcccccacttcttcacgctcaacggcattaacgaactgctgcgctgacattccggagaggatcgccagcgccaacttcgggccgacgccgttggttttgatcaactctttgaacaacgtgcgctcttgtttattgttaaaaccgtacagcagttgcgcgtcttcacgcaccacaaagtgggtgaaaacgatcgcttcctgacccgcttcagggagttcataaaaacaggtcatcggcatatgcacttcatagcctacgccgcccacttcaattaacaccagcgggggttgtttttcaatgatgatgcctctgagtctgcctatcacatgacgctcctgcgtaatgaatcaaagataatgctgtatgataaaaaaatgctggatagatatccagcgaaggatgaagaaaacttgcgaggtgtctcgatgatctgaaaaatggcgcagtataatttattctacagattatattggaagcaaatatttaaatattacatattcagcgaagaaatgtgtaataaaaatacacattgcgacccctgaaaaaaataaattttttatgctattacgtatattcatatctatttcaatggaatgacaacgtgaatattaattatcctgctgaatatgaaattggtgatatcgtctttacatgtataagtgctgccttatttggtcaaatatcagctgcatcaaattgctggagtaatcacgtcgggatcattatcggtcataacggtgaagactttctggttgcagaaagccgtgttcccctctcaaccatcactacgctatcccgttttattaaacgctctgctaatcaacgctatgctataaagcgattagacgccggactaacagaacaacaaaatcaacgaattgttgaacaggttccttcccggctacgcaaaatttaccacaccggttttaaatacgaatcttcgcgccagttctgttcaaaatttgtttttgatatttataaagaggcgctatgtattccggtgggtgaaatagagacgtttggagaattgttaaatagcaatccaaatgcaaaactcactttctggaaattctggttcttaggttctattccgtgggagcgtaaaaccgtcacgccagccagtttgtggcatcatccgggtttggtgttgattcacgcggtgggagttgaaacgcctcagcctgaactgaccgaggcggtataacttaacgcagtcgccctctcgccaggttcagtcgcgattcgctcatttgcatcgcattctgactaacgtggcagtgggtgatggcaatcgccagcgcatcggcggcatccgcctgtggattagcgggcagtttcagcaaggtgcggaccatatgctgcacctggcttttttcggcactaccaatacctaccactgtttgctttacctgacgtgccgcatattcaaataccggcaattcctgattcaccgccgccacaatcgccacgccgcgcgcctgccccagtttcagggctgagtcagcgttcttcgccataaagacctgttcaatggcgaaataatcaggctggaattgggtgatgatttccgtcacgcccgcatagatgagcttcagacgagacggtaaatcatccactttggtgcgtatgcatccgctacccaggtaggacagttgcctgcctacctggcggatgacgccatagccggtcacgcgcgaacccgggtcaatgccgagaataatagccatcacgcgtctccgttttgctgtttagcaggcctcatcagagagtcgctgcaacctcatcagagatttcaccgttatggtaaacttcctgcacgtcgtcgcaatcttccagcatatcgatcagacgcatcagtttcggtgcggtttctgcatccatatcagctttggtggacgggatcatggaaacttccgcgctgtctgctttcagacctgccgcttccagagcgtcgcgtactttgcccatttcttcccatgcagtgtagacatcaatcgcgccgtcatcataggtcacaacgtcttcagcaccggcttccagggctgcttccatgatggtgtcttcatcgcctttctcgaaggagatcacgccttttttgctgaacaaataagctacggaaccatcagtaccgaggttaccgccacatttgctgaatgcatgacgcacttcagcaacggtacggttgcggttgtcagacagacattcaatcatgattgccgtgccgccaggaccgtaaccttcgtagatgatggtttccatgtttgcatcatcatcaccgcccacacctcgtgcaattgcgcggttcagagtgtcacgggtcatgttgttagacagtgctttatcaattgctgcacgcaaacgcgggttagcgtccggatcaccaccgcccagcttagccgcggttaccagctcacgaatgattttagtgaagattttaccgcgcttagcatcctgcgcagctttacgatgtctggtgttggcccatttactatgacctgccataaaaatatctccagatagccctgcctgttcaggcagcgttaattacaaactgttcaatcgcctgccggttgctccaggacttagtgagcgccgccgcagcagacgcatcaagccacttgtaagccagatgttcagtgaaaacgatctggcgctcgtgcggaagcgcaagacagaaccatgattccgtattacgcgtcacgcccggcgcatagcgatgacgtaaatgtgaaaaaatttcaaactctaccgtgcgctgacagtcaattaaggtcagttgttcagcgacaacatcaatggtgacctcttcctttacttcgcgcatggcagcttgcggcgcggtttcaccctcttccacgctgccggttaccgactgccagaaatcgggatcgtcacgccgctgcaacatcagcacccgtttcgtatcttgtgcgtagatgaccactaagatcgaaacgggacgcttataagccatatcagttattctcagccttcttcacaacctgaatgctcagctcagccagtgcagtcgggttagcaaagctcggcgcttcagtcatcaaacacgctgccgccgtggttttcgggaaggcgataacgtcacggatattgtcggtgccggtcagcagcatcgtcagacggtcaagaccgaatgccaaacctgcgtgcggcggagtaccgtatttcagggcgtcgagcaggaagccgaatttctcgcgctgttcctcttcgttgatacccagaataccaaacaccgtctgctgcatatcaccattatggatacgcacagaaccaccgcccacttcgtaaccattgatgaccatatcgtaagcgttagccaccgcattttccggtgcagctttcagttctgctgccgtcatgtctttcggtgaggtgaacggatggtgcattgctgtcaggccgccttcaccgtcgtcttcaaacatcgggaagtcgataacccacagcggtgcccatttgctttcgtcggtcagaccaaggtctttacccactttcaggcgcagtgcgcccatcgcgtcggcaacaattttcttgttgtcggcaccgaagaaaatcatatcgccatcttgcgcgccagtacgctccaggatggcttcgatgatttctgcattaaggaacttcgctaccgggctattgataccttccagacctttcgcgcgttcgttaactttgatgtaagccagacctttcgcgccgtagattttaacgaagttaccgtattcgtcgatctgcttacgggtcaacgatgcgccgcccggaacacgcagagcggcaacacggcctttcggatcgttcgccggacctgcaaatactgcaaactcaacagatttcagcagatcggcaacgtcggtcagttccatcgggttacgcagatccggtttatcagaaccataacggcgttctgcttctgcaaaggtcattaccgggaaatcgcccagatccacgcccttcacttccagccacagatgacgcaccagcgcttccatcacttcacgcacttgcggcgcggtcatgaaagaagtttccacatcgatctgagtaaattcaggctgacggtcagcacgcaggtcttcgtcacggaagcatttaacgatctgatagtagcggtcaaagccggacatcatcagtagctgtttgaacaactgcggggattgcggcagcgcgtagaatttacctttgtgcacacgagaaggcaccaggtagtcacgcgcgccttcaggcgtggctttggtcagcatcggagtttcgatgtcgaggaagccgtggtcatccataaaacggcgcaccaggctggtgattttagcgcgggttttcaggcgctgagccatttccgggcgacgcaggtcgaggtagcggtatttcagacgcgcttcttcggtgttgacgtggttagagtcaagcggcagaacatctgcacggttgatgatagtcagcgaggacgccagtacttcgatttcgccagtcgccatatcgcggttaatatttttttcgtcacgcgcacgtacggtgcccgtgacctgaatgcagaactcattacgcagttcagaggccagctttaacgcgtccgcacgatccggatcgaaaaatacctgcacgataccttcgcggtcgcgcatatcgatgaagatcaggctaccaagatcacgacgacggttgacccaaccacacagagtcacctgctgccccacgtgggacaaacggagctgtccacaatattctgtacgcatgagatatcccttaacttagctgccggcggatgccccctgctgcgcaggtgaccaagtcgcagcgttagctgtatgtcacaactgaatgaaaaaaggcggctattatactggaaattctgccgcaccgtaagagcctggcccgcgctggaacgcctcgttaccactttatatcgggcctgaaatcagactctacgccagtttgctataaaggtgttgcccgaactcataaaaattaacaaaatttgtcgttccgccatcggctaatcgcattaaggtgagaggcacgattttgttttgtcaggagtcatcatgcttgaacttaatgctaaaaccaccgcgctggtggtgattgatttacaagaaggcatcttgccttttgccggaggtccacatactgccgatgaggtggttaatcgcgccgggaagctggcggcgaaatttcgcgccagcggtcagcccgtgtttctggtgcgcgttggctggtctgccgattacgccgaagcattaaaacagccggttgatgccccctcccccgcaaaagtgttgcccgaaaactggtggcaacatcctgctgcattaggtgcaaccgacagcgatatcgaaatcatcaaacgtcaatggggtgcgttttacggtacggatctggagttgcaattacgccgccggggtatcgatacaatagtgttatgtgggatctcgaccaatatcggtgttgaatccaccgcccgcaatgcctgggaactcggttttaatctggtgattgccgaagatgcctgtagcgccgctagcgccgagcagcacaataacagcattaatcatatctacccgcgcatcgcccgtgtgcgtagcgttgaagagatcctcaacgcgttatgatttacatcggtttgccacaatggtcgcatcctaaatgggtgcggttggggatcaccagccttgaagagtatgcccgccactttaactgcgtgacgcgggcattttaaaaatcactaaagaacgcccaagagcatgtgttttctttagtttattcaatgcattaaaaaatagtttcgcatgaaattcggtaaacttcatgtgtgcaataatgtcccattcatgccccaaaatgccccaaagcagacatttttgccccaagtatgccccacaagtcacgtcttcaagtcgtctatatccatagcacaccgagttacattcttgcatccggggtgtcgacaatacctactttattgagtgtgcgagaattaccaggaacctttccacaatgtagtagtctaatagtcgaatccatctaacattaagaagcgttatgatcactagcctctcattgatatcttctgtaatagtcactctatgtatcatggtgttcgctacagtaaaggtagggattggtttgtctaacaatccagacagaaatgataattaacctcaaccacgtaaccacacttcatacttcatacttcacttaacagtgaagtgctcacatcaccgggcagtcatcaaactccgcattcctggcatcattaatgatgtacgtgatcactccaaatatagcgggtgcagaactgtaaccatcatcatctgctggcagcgcttcccttctcccgttatccagattaaccaggtgcggctgaggatgagtccgatatcgcttgatcctgaattccccgtcgattgcacatatcagcagtgaaccatcgcaggcagtaagtgacgcatccacaacaagcaacgctccctggattatcccttccctgaaatgtgaacgcgatgcccgcatgaaataagtcgctgcgggctgactgattagctgctgatcgagggagattcgtgtttcaacataatctgccgcaggtgaaggaaatcccatgtttacgccctctcttgaataccggataaaaacacagtataaatactgtatatccatccagcaaagaggcaatgagcaatgttcgtggaactcgtttatgacaaaaggaattttgatggtctgcccggtgcaaaagatatcattctgggcgagttaactaagagagttcaccggatcttccccgatgctgatgttcgggttaaaccgatgatgacactgccggcgatcaacactgacgccagcaagcatgagaaggaacagataagccgtactgttcaggaaatgtttgaagaggctgaattctggttagtgagtgagtaaagattttcaatgcccgccacagttacgtattgattatgctgtggaggatattcattttcgtaaacgttggtttgggagaagcggcaaaacggaatgtgggaacaggggaaaatcagataccagatatgtctgcatttccatctggcaataactggtttcagttaccaagtggacatatcgttcagatattttccatgaacgttcttggtgcagatgctaatggcacgtcagctaattaccccattgcttttccaacaacgatgattgctgtcagtgctctatggtctgatgggactgtagcaaatgcaccgacatacaagatgatggggaacacgactaacagaacaactttgacgataaaagtatcagccagctcaggtacttacgggacaatgattattgcggtgggacgataatatgaataaatacagttactctccttcagaaaatgccttttatgctgttgcgttaaaaaatacctatgaattgagtggcacatggccagctgatgcattagatattcctgatgacatttctgtaaaatatatggcggaaccgccacaagggaaaatccgagttgcaggggaaaatggttttcccacatgggctgaaatacctccaccatcacatgaggaacttattgaacaggccgaatcagagaggcaattattgattaaccaggccaacgaatacatgaacagtaaacaatggcccggtaaagccgctattggtcgtctgaaaggcgaggaactggcacaatataattcgtggctggattatctggacgcactggaactggtcgatacttccggtacgcccgatattgaatggcctacgcctccggcagttcaggccagatgacatccggcgcggtgctggtatctgttgcagtcaccgcgtcaatgtaatccagcacggcgttaagtcgggttgtttctgcctgagtcagtttccgtccggcctgtaatttcagctgaatcagactaatggaagccattgctgcatcaatcagtgattggcgctgtgcttctgccgcttctactgaggcaccgtgttgtgcctcagtatctgtcacccatttctcaccatcccatttatcatatggcgttaacggtgaaagcgtgacataaccgtttttgatggcaccgatataatccactgtaacagctgcgccattttcgattgagtaaacagtctcattgcgatggtcttcctcatggctccatcccttacctgtaaatactgccactcttcccggaatgttttcgtccgggtcaataccagtggaacaggcgggcatacttacgccagtattaatatattcatcagaccagcccgtatattcagacgttactgcatcataataaaaacaacgcatatcacccggcactgcagccagcccattttcatcaaaaacaggtttcattatttagccctcaccagaaagttaaatgcaatatttcgcggtctgacagcaacaaaattcacaccatcacccacagagttactgttgaaattaaatcgtgaaaatcctggctgatttccggcgatgccatcatgaaagttaattgcgtgtccagcacctccgcctatattcccggcaaactgagaaaagtttgtagcttcctgccagcttaataattcgcgaccaccatctgcacctcgcccgtcatcccagacacgaatgaaatcaccgcgggcttcaggtaataccagcgaaggaaacactttcgccagcacaggataatcagtggcagagaatttcgcgccgttgaacttcaaaaacaccatactggaccagctgtcgattacagtatttggcattgcagcggacggccagaagaacggaacgccaatagctggagaaccttctcccaaaccaaggtttgtgcgagcgtctgcggcattcgttgcgccggttccgccgtctgcgacagtaaccgcaccgttgctccctttctgcgcaagtttaccgatgcctgggatggttacggcggtgccgttgatggtaactgtgatgctttggtttgctgaggtggtggcgaacgtctcccacgcgccaatattctcgtcgtactctttgatgagctgtgacatggcctgcgccaggccgtcgactgagatattgtccgacacaaggattccatacttctggccgctcagcgccggggaaactgctggcgtaaccgtcattgacgtggcgctgttcacggatgaaatctgaaacagctgcaccgggttagacatcacgataatcgtctggccagcgcggacctggctggcgggagctgtccagtttgtgccggagccggttgcggtatttccgttaatagagatagttccggtgctataaatcataacaactcctaaatttagacaacatgaagcccggagaggtatataaccctcaccagaaataatttctgaattggtttttaatacatgttgggcaacgccagtgttggcatagctatagttgtatcaaatgccattgaccacccacccaaataataattgccgactactttattcctttctgctctgacattcccacctgtcattacaattcctttatacctaatatttccgtaaccaccaacgtgtcgacagttagccccggtataaacaatctggcaaaattcatcaccaatattcaggttcgcattggcgatatttattgtcccatcaaaaatgaatggcttcgtgcaagtaagaagacgtttgcgttgacgattggtttattcaatccatcaactaaaatatcatgtaaacgaattgccataacatttcccactttattttacttactcaacacaacaagtatattaaaattgaaccttgtcaacaacacaaaggagtcccaatgaaactcgctctaattatgctgccattatgtctgtccctcactgcatgtggtaatggtttaaataccggtaaaccaaattccggtgtcattccaaaacctttggatcgagatggtaacggttctttaatttatgataccgaaaaccttccaatgacggggcagtggtgtcacgagattgatcacgaataccgacgaatcggtagcccttctaactgtgttatagactactaaatattaacccctcaaaagaggggttaatattttaacctgtgaatgaaccagatccgtgtgatactatgatagtaggcgaagaaattgacgcacccctgttattttgagcggacactttaatacgcactgttacgtttggacctgttacaaccgcagaatgcatgataagtctctccatgtcttgcacggatgaaccactctcattgccgttaatgtcgattgttcctgcaccattacctttaacgtttgctataacacagacgtgtcttgcgtgcccagaactggatgaatcattataagtcattaccttttctaaatatccgtcactagaccgactcacattcgtccctgtgtgcatatttgcaatgtccccgataaaactttgggcttccacagtccctttgaatttaccgcttgttgcttggatctcaccagtaaagctaccgccactagcatatactacacctctgacggtcacattgttgaattcagcatctccagctttattcaacttccaaccagcagaaccagctgcatagttgttggactggatatagttaccgatttttgcgttctcaatggtgccgtcctggatgaagctggcccggatgaatgtctgcccgttctggatcacgaacggcaaagctacgctatttccggctgccgtggtgacggcgaagcggtcagccaggaagataacctgcgactgcatgccggatggcgtattctccacgccgatacccatccccgcggcgtaatactgcccgttgctggagacaccaaccttgatgttgtacatcgcgctgagttcgccattaacgttggctatagcctgagcgttagtggtgatggcggaggtatgcccgttcacggtcgccgtgatgccgtttatctgcgtggcggtggcctgctgatagtcggagagcgtctgattcaggctgttgatggatgccttgttgccgttgacgtccgtctgcaggctcagcaatgaacgtgctgtggcttccttctcactgacgatcacctcgtcgagacggtccagattcgcgctgttgccggcgaccgatgcagaaagggttttacgcgtggccacctgagcgaggttggcctggattatcgcaattgcagagttcttcaccccgcccgtcatgccgtccatagaaacgctgatgttgtcgattcgctggcccagggcggtatcagccgtcgcaacggtctgctcaagctgactgagtgaagacgaaacattcccgaccgtgctggaaagctcattaacgctggtctgaaccttcccgacgtcctgggcatttttggcgatatctttcgcttgctgctccagttcgtcgttggcctgtttgatatcgttagccatgccagcaattttttcgttgctgtccaccgcgttctcgatcaggtctttgaacgtttccgactctttcatatcctccagaatgtcattagttatttcgctgacatctatcgaggacgtgcccatgatccagtcggtccagtccccggcgttaccgatacggtcaatcaggcgcgcgcggtaccactggcgaacgccggcaggcatggggccatgctgataatctgcagccgggtacggcaccaggaccagcagttcaggattggcgtagtcggcagttgtggcgcgctgaatctctgtataggccgtgtcgcctgagccatccggaaatttccaggtcaggtcgatatgccagaccacatcttcggtcgccaggaagttgagcggagtacccggttttcccgttttaccggagagataagttgtttcaccgtatccccatggtgacgacgtatcctgcgcattcagcgcccgtacgcgcacgtcatagctgcccgaataaatgccctgaaccgagaaaccctgcgcgctggtaaccggaacgtttatccagtccccgttgtccttacgccactgggcaacataccggattgcgccctctaccttatcccatgacacgtccaggcttgctacagtcagcccctgagacacatgatcgctctcagtcaccacgatattcttcggagcagacaggacgcttatcggcgtgacggtgatcgggggagactcgacccgaacgccgtcatcgatgtaacgatatttgtttggatcgtgctgaacggccgtaatagtgaaaccgcctgtactgtcgtcgttagccgcgattgaggtgaccctgaagtactgtattgcgaggttatcactgtctatcgcccaaacagcgcccgccacaggaacctgactgaatgccgtagccaccgtcaccgtttttttatcggcgctcaccgcgctgattgtccgcgtctgggcttttccgtcgggaaggttaaccaccagccggtctttcgccgcgtagtctatttctcgatcgagggtaatttggcggccgttggccgcgcttatacggcccccgttctccttaccagagcggaaaggatcggcgacaccgataatttcagcgggcaaagggatataaccgtccagccccacgccaaacgatacggtcccgtctttggcattggagagcaatacccagcgaccgcgtcggtgcgcttcactttgcgaggtgcagccgattgcggtcagggacgtctgccggacgtcgtaacgttctacaagcgccgaatcgtaaaccccctcaacggtatcgctgtaatggttctgcggatcggaccaggacaccaggcaggagctgtagcgattcttgtatgagccgcccgcataagtaaacagcccatcgataacgtttgagacgttataaacccagtcaacatcgtcctgcgggacgtctgcctggacataaatctgatcgtttccccagaacgttattccacgaaataccgcggcgagatcgttaagtacctgccaggcgtcctcctggctctgaatgaaaacgttgcaggtgaaacgcggttcggtgccaccggccccgtcggaaaccatttcgtcacagtactgggcgattgaatacagcgcccacttatccaccatggacgcatccacgcgcgtgcccatgccgtaaatttcatccagaaccagatcgtaaaagatccaggcagggttattggaccatgccattttgaacccgccggaccatgaaccagaataggttcgggttttcggatcgtaattatccggaaccttaatcagcttgccttttatcttacaggtcactttcggcgcgctgccgttgaattggctgctgtccacttcgacatacaggagcgctgttaaaggataacgaagcttgctgtcgatgacttccgcatacgaaaacaccttgaaggcgttaaccagtttcgaatttgatccgctggcatcagccgtaatacgcctgaccctgacagaccagccggacgtggattttggcagatcgatacggtggtcacgctgatattccgtcgtggtctttccgtcaaacttgccgtttacaaccgttttccaggcgccgccgtccgttgataaatcgatcgcatactcggtgaccgtgcccaccatatcgccattatctttatagagatactggaccggaaggctgagcttgatacggatggcatccagggaaaggtttgtaaactggcgcgtccagggcgcggtggtggtgacagttgtgcccacggccagctcgttgtcgacctggggcatcccggcaatataggtctggtcctgtgtgcccttgcggaactcccatttcacgccgctgaagttgtattccccgctgtcgtttgccagcggcgtatcgttgagaaaaatgttctgagcggtcaggtcgccctgtatttccccctcagaaacggcaatgagcatttttaattttgcgaccgacagcagatcgtcaggctgctcaaccggagtatgtgaactgccacctccccctttggcaccctgcaggatggtttcttgtttaagaagctgcattttttcacccataaaaaaaggtgccgaagcacctttaagttagtggccgctggcctactgctgatcgctcgagtacataccggcgctgactatcgctccccctgcctcagtcagaccgtaggccagggggacaggatgccccatagcgacggtattgaccggcgccccgaaggcgtagttaggcgtgttgtccgtgctggaggatttacccgcgccgaaggatggctggggcgtgagcatctggacaacgccccccagcatcatcgacactccgacccctgtcagaattgacgtggcgctgatagctgttgcactcatcgccgcgccccaggctgccatgctcgcaccagcggtaaagaatgcagcgaccagcgcaacagccccgacaactatctgcaggacgcccgaacttttggccccctcataaacgggcacgatccggtacacgcttccaccgcgggtcatatcaaactcttccagcccgatattgttgccaccgttaaaaaaggcgaaacggatccccttcatatgagcttcagacatatattttttgaatccgggaacctgtgaacacatggccctgagcatctcgcgcaggtcggcaacatcaaactgaacgcgtttaccgaatttttttgccattttaccttcgagaataagcgtcttaaccatgcattctgtccttatgcctgaccacccggaccgttctgtcgcgataatattttccataaggcgttcgcgaagaaaggtgcccgaaaagatgatggagaatgatgttatcacccacatataccgcggcgtgattagtcaccgatgcctgcacactcatcatgatgatatctccgggctgcattgcaccggcggcaatctcaacgaatccctcacgctcccagttgtcgtcgtagagacgctccttgccgctctcccaccattcgtaaggtactgaataattgccgagaacaatgccgtattcgcgcagataaaattcacggataagcgaccagcagtcggcgtaacccagcacccactgccgcccggcataatcccggtcttcacgcggggaaatcgtacaaaaatccccgtccggccaggacatgatcccccactcaatccccgaccagtcgcactggatccggtccagctctgagggcaccagccgaaccacatccggatgggaatgaatgagcatgatgatctcaccgcgcgcgcgggcagcgagctggtcttccggggagagcgtgaatgtctcctcgggtttatcggcaatgttgcggcagggaataaagatttgttgctggcctgactgaacaatcaggccgcaggcttctttggggtattcagcagcgacgtgctgacggatagcatccagcaatttttcacgcatttttatttcccctgcaggtttgcagccggaaaaccgccgaacggcagcggcgcatccgggccgtgacgatcctgacaatcctgccggcggccgccacaaacatctttcgacgggtcatcggtcggtgtaccgtctttggtaaagtatttcgtgccgttgtaatcgcatccggtcccgcttcggtaccagccccgcatacaccaggtgcagacaggcgtaatctgccgtgtcggcagctgcaggctctgaatatcgaaaggagaacacagctcgaaatcaacctgtacccgcgtctctgcggttttagcattgacgtaaaagagctgtaagcgctcatcggccgggctggcacccggattaccgtttttccagttggcggcatcgagatacttcgaaagcgtggtatggattttgaccttagccctgaccatatcgtcatattcaagacacagcgcggtgacatagtttccgacgttcccgacggacagcgtgggcgttggctgggaacctgtactcgataactccatccccttaagttcgtagggatggggatcgtactggtttccctgccagataatggcgggcagattttctgcggcgaaggctgcccacccctcttcctgaatattgtgcgcatgaaaacgcagcacctgatccataccgaattcagtgccgtcgatctcaatcagctgaataacgctgccgggctcaagctgttgtatgtctgccgtaaaactcatactccccccataaaaaaaagccgcccggaggcagctttcagtgtttttcgagaaaatcagggcgcgaacgcctgttcaaaagtgaaggccacagtggcttttttcccggtagggaaagaaacgctgaacgaatcggccttcattctgaacagctttttttcaccccatggagtggtccaccagaacgatttagtaacgtgagacatcaggaaagcgcgcagcgcagccgcctcctgtctggtgcccgtccagtccaggttccacgtttcctgtttgtcgttgatccccatccccgctatctgtttgtagccatccccgaactgggcctgcagcgttcgggctgtttcagtgccctgcgctgtttttcgcgtgcgccaggtaaacgtgtccgtcactgtgtcctcctcgaataaagcacgccgcccgcggacatttcttttttcagtcgctcggtgattgtctgctgaacaatcgcctgcagctgtttcgccgtccccgtggcgttcgcctgatttatgcttccgtcactcccctgctggctgatgctgactggggcataaacactgatcccgcccatgccagcaccggctgcgttcccgccgccgaccagaccacccgaggcatacccgcgcatcaggcgatagagattagccacgccgatgcggctggttgattctttggtgaagacgaattccccgcggtgaacgataccggctggctcgtacttgccgccgtgcccggtaaaaccgcccacgtcaaaaccctgtggccggtatgacgggaccgcgaatgactgaccggcagaggaggttttcgccccgccgctaacccagcccattgcactctggatggtgtaagccaccagcagctggttgataacggacacaatcattttaaggatcgagctggtgaattccctgaagctcgccttcccggttgtcgtcaggctggtaagctggcccgccaacccgctgaacgtagcctgagaaatctgctgaacggagctgaaaacgtttgtcgctgaatcctgatattcggcccaaccctgtttcgcaccggccagccagtttgcacgcagggcatcttcagcttcgaacgtcgccctttgctcttccagaaccttttgctgcgcctgagggttgtacgaatagctttcgctgagacgctgcagcgtagtttgtcgcccggcttcccgggtggataacccctcagactgagcctgcaggcccgccctggcggctttttgctgctgctcaaacttcacggcctgatcggccagctggttgagcttttgctggctggcaaccttatcgcccaggtcggccagctgccgcttgtactcgagcgtttcttctttgtgcgccagcagggatttttcctgcgccgtaagctgacgacgcccagcggcctcctgcagaacggtgaactgattttcagtttgccagagatcctgacgctgtttacttatgacgtcgttcacgctggtatgctgctcaagcgttttaagctgggcctgaagggtgagaagttcggcctgcgccttttcctcggctttgtccccggcgggcgttgagtagcttttgcctttcggtgtttttggatccttccactgcttttcaatcccggcgcgggccgcggcaatgtccttttcagtccacagcgtggcgacaccgtctttcgcatcctggcggtttttctcaataagctgactgagctttttctctgctgaagcccgcttttctgccgccgtcgcgccggactccaccagctggttaaactgctgctggctgcggattgcctgagcctgctggtccgttcgcattttttcccgcgcggctgccagcccttcctgggcgtattgctgatcggcaagatcgtaagcctgctttttcagctccacctgctggcgcgcgtttctcagcctttccgcatccgctttctgcagaacgttgttaccggcataatccgggtcgaccttaagattgctggacagcgcgcggtactctttctctgctgcctgccactcagcaaaagagtcctggcgcttcatcgcggtgtcaggattacgcccgacgcccagcatcgcatcccacgcaccggaggcggcattcttcacccagttccaggctttttcgagggatccgagattatcctcgaccgcaccggcgcgctgaatgaccgcgtcggaatatgcccgcatggccagctcggcagccttctgagaatcccccagcgcctgagcagaagctatctgttcatactgggtggctgtcagaaaatgaagggaatcgttgagcgtcgcgaccgcgttaaccggatcatccttcaggcgtttaaactgatttatggtttcgtcaacggcctgcccggtagcctgctgcagcctggcggcaacattgctgaccatgctgacgtcattaccgctgaacgcgccgctgccaacgacctgcgccagcacgcctgcagcggcatgctgagtgatgccattacctgccagcgagcgcgccagcgcctgcagctgccctgacgttttccccgcgtagttcccggtcaggatcagctgcctgttaaattcctcagactctttgctgccgtcgtaccaggccttacccaacccgaataccgccgcggcaatccctccgaccatgctggcgatcccaagaccgcgcagtgacagaagctggtctatccaccctgcccggttagccagcgtgatcccggagccgcgcagcgcgccgaagttaccgcgcatgacctcgccgatcagtattcccagttcctgccgggcggcggcactttgcagccccagaccgtgcgtggcgactttggcagcttcgagcttgcggatatagacttcagccgcatcgctggcaccgacctgcgccgccttcatgcgcagtagctcggtaccggagagcttttgctctgcaacctgttgcttcagctggctgaggaatcgcgtgcgcgctgcggccgatttttcctccacgatctgcagttctttttgacgggccgtggtgcgggaaataagggcgagataatcctgctgggttatgttgccctgtgccctcgctgcgcgaaagcgcgcctgcacgttcgcaagcgactgtgtttcaccattgagctggcgtacgccgtcgatctggcggaaaaatgatgccgcaagttcatcctgtcgacgggcaagcgcagcggcctgcccgtcattctcacgcatgcgctgattaagctcggtcacgcggcggtgagtttcatcaacggactttgaaacgttctgccagtctttggtaagcccttccgttgcggccgactggcgggatttcatatctgcggcagccgccgcgccagcgtcacccacggttttaaacgcagccgcctgccgctctgaagcgcgctgcattcgcgtctggactttttcagagtcctcagccatcccggttagctggccctttatgcgggcaacctgctcactaaacgtggcgctgtcgacgtcaaggttgatgaccagatcgctaatctgctgggccatatcggatacctcctgttatcccctcagctgcggccatcagcgcatcatcatccggctcgtcatcgctgatgacgataccggaaggagaaagcaggctgaaatgtgcgggggtaagttccgggtcgcggaagaaaagagtggagatggaataaagcagctctgagaaatgcgcatcgagctgagcgtcctgaaaataatgctcccggtagaactggtgccagtcgcccagctcagtggaagtcattccagccagcatggcgcgccagtcgggtcgcccgaactcgcgcgccagattcaggacaaacttcagctcgctggcaagggcttttccgccgcaacgggttctgcgctttcggcctccgctggggcatccggatcggcagctttgtcatcctcaaccggaacgagcatgccggagagcagctttatttccatttctgctttaccgatcgcctccggcggccagccgctaagcacctgctggtaaagcgtctccacatccgtgccagccggatcgttatgccacaaagacatcgcaatcaaacgcgcaccgcagcgaatatttgagccaatcagcctggccgtcatttcctgatcgctgatgccgtcgctgtcagcgctgacggccttttcctctgcggccataaacgtgatgtactcaatacgctgaagcgccgacagctcgaagatggtcagtgattctttttgccaggtgaacttctcttttttcagaaacatgcgtccttccttacgctgcagttacggtaactttgcagaccgcaacgaaattaccgtcgctggtcataacaataacgtcagcggtgcctgccgccacgccggtgacggtgatcgcattaccgctaacggtgaccgttgcttttgccccgtctgaggttgccacacggaacgaggtatctgaggcactggctgggttaaccgtcacattgagcgttgtggttgcgccgacggccacgcttgccgtggctttatcgagcgtaacgcctgtcacggggatattcggggtcccgctttcttctgccagttccggcttgccggtattggtaattttcgctgtacgggttatgacctcttttgccggaatggctttacccaggctgctgcaccagccgcggaaaacgtcgacggtaccgttcgggtatttgattttgtaatagcgtactgagccatcaataaaccatgcgacaaggtctttttgcccttcttcgcccggcttccaggcgagggtgaacgaggtatcgccagcagattttgccccctgggccgtcgcgttccagtcggcatcctcgtcgtcgaggtaagtgtcgtcatacgattcggcggtcatttcgcccggcgtcagctctttaattttcgccaggcggttccagtcgatatccgagagtgggttagcgaaagcgttgcccgttccggtgtaaagccagagggtggtaccggcacctttcacaggggccagcgggtttggagtaggcataagtacctcttaaattgaataggtgattaagtacgtgaaatcgactgaaccccaggtggccatttcatcatcccgctgatagtcataaccctgcggggtgaacgtctcgaccagttcggtcagacctgggatgaaggccattgccggatacactttctcttccatccaggaatcaagcgcgctgtcggggctggaggctttaagaaatacctcgatgtgaacaaccgcctgccacgaatcttcgtcaagcgattacacgtcttgagcgattgtgtaggctggagctgcttcgaagttcctatactttctagagaataggaacttcggaataggaactaaggaggatattcatatggaccatggctaattcccatgtcagtccagtgattctgaataggcgataagtccggtataaccggggataatctcaccattatcagcttcaaattcaggaattgtgccggtggtgatggtgtattgaggctggccatcttccttcgcgaaggctgccaggtcttcaatctgcttagctgtaagaactactgtcatgctcattcctcagttgtaaaaaagccccgcgagtgcgaggcgatttgattgaattctcggctcttatctcagcgcagccccttactgcgtgccggttgctcggtgatgagcatcagcgatgagacattaaagccgaccgaaggccagcggcgttcctcatgttgccgacagagccatatcgacaagaggacgaaaactagcagcatgaatcgcctattggttattcgacagtcgcactgattcgtaaatccgctcacacgtcattcctgcccggtagctttcgtcagatcgtccagcataatatcgagctgcttctgcaaggcttccgagcatgtcggcaagcattgctgcgttggctccggctgttttgcttctgacggaagtggcgagatctgcggtgtgctttgcggcgtccatgtgggtagcgagttttgttgcttcggcgcgcagctgcttaacagtggtagccaggccagcagaagtaacggcagcgctcgctgcttgagcttgagcatctttaacggcctcatcccgggcgattgttcgcccttgttcaatcatacgagctgcggtctgtgcattcgcttcctgtgaagattccgcgctatcccggtcagcccactttattttccagctgcggttcgtccactcactgccagcgagaaacgaacctaccaacgcaacaatcacaatgatgcagatgccacccggcttcactggtctatcccccagcacgtcagtgcgctttcctggtcccgccgttctacctgcccataacatccatccttctggcccttggtcaggcggcaatcgcggccaccgtctttaatccaccagcggatagcttcacaggctcctttcgtatcgccagcattaattcgcttatagaacgtagacgggaaacattttccggggccgatgttatatgggcagaaagaagcgatacccgctttctgtggttcggtcagtggtactttgatatttcggtcaacccacgccagcgccttgtcgcgttcaatggcgtttacctgggcgcatttctcagctgacagcttcatgccctgtactactggcttgccatcaaccattgttgcaccacggcaaatggtccagagtccgccgccgtcgcgatatgctgtcaagctgttaccctctttctcatccagaaactgatcgagaatcacgggtgcggaagccccggcaagaatcaaaccaacgaccgctgcgctcaatttattcttcagctttagagacatagccattgcgccgatcctcccgttctttccagcggaaataccagttcactgcacaggtgattaccgtgcatgcgataccgacaataattgcccagtcgctcaggcttaaccctgcaattctgtcggccaacatccaggacacctcttttgctgttttagctgtttcggcatatgccttcgctgatacaccgcagccggcaagcgtggttcctgatccatatgaaagtctgctgtaaatggtgctcattctggtcatagcctcacctccgatagttcggatggcgctgtgtgtgattgaaggggatcaggcaaccgggctcttatgttcaagtaaaaattaaggatgattcccggtgcctgaagatggtgatcaccacagcaacgggggagcgtggtgatcgttatgattttttcagtttttccacctcttcggtggtctgtataaacctgtctgcctccagttctacgccgatcgcccgacggccaagttctattgcagctttcacagttgaaccagagcccataaagaaatcggcaacgatatcccccggtctgctgctggcgctaatgatctgtttcagcatgtcggcaggtttttcgcatggatgtttgcctggataaaactgaacaggcttatgtgtccatacgtcggtataaggaacaagagcggaaacagagaagcagcgccgaaggattttgtattcctccagcaattctgaatacttgcggtttaatgactggtaggtagccaccagctggtggtgaggatgttcaagcttttgctgaatatgtttatcgatggcgatccgcgtgaacagttcctgcaattttcgatagtccacttcattcggtagttgccattggcttgcaccaaaccagtgtgacgccatgtttttctttccggttgcctcagctatttctttcgagctgacacccagtgattcacgggcattacggaagtaatcaatcagcggcgtcataatgtgctgctttagctctgtgcttttcctttcgtaaacatcctctttacctgtatacggtccaagatagtgctcagcaaacaaaatccgttccgtagatggaaagtacgcacgcaggctttctttattacatccattccagcggcccgatggttttgcccaaatgatgtgattcaaaacgttgaatcgggcgcgcatcataatctctatatctgaggccagtcggtgaccgcaaaacaggtagatgctgccagcaggtttaagaacgcgagcatactcagccaggcagctatcaagccagcgtaagtagtcctcgtcccccttccattggttgtcccagccgttgggcttcactttgaagtacggaggatccgtaactataagatcaatagagttatccgggagggtggcgacgtaatgcagactatcagcgttgattaactcaacactgtttatttttacagtatttttcatagatcagtaagcgtaactctgataggctcacgttgcttttgcgctaaagcagtgggccttggttagcttgtgacctgaaagcatgagctgatggctggccgggtgcgctaacacccaccagccgcccatttccacagcagaaaacccccattactggaggcgtttataacatccgaactggtaatcagataaccccgccatcaccagctgcgtaagtatgagctggcaacgttcgtggctgaggtgggtattctgtgcaatctccccagccgttgctggtttatcgcttaattcattgaaaacagcctttgccgtttctgtcatatcttcctgatttagcatgtcttttacctaaaattagttgcgtgacatacagataactctggttggtgataccagcaagagaagaatttgattctgcaaccaacaaggcctttaggcatcaggcaggaatgagatgcaataaaaaaaccacccgaaggtggtcttatatgaatctttaacgcggacttagcaaatattccacatcatcgtactaccgttatggttttcgataatttttgcggctgggctagtaccaaaagagtgcatatagcaatgatgaatagtaaggaccagatcctgcaacgtttggtcactctctagctccatgatatttaaaccaatattttgagctttgtccaaatgaatatgtctggcatgtgcatacgttgcttggtggttgtttaactcatcacatatacgcttagccttagcttcagcgtcagcctgacctgcgaacataccagtacaaagccatttctggacaatttcgttcgcccagagaattgctttttcacactcgccaatcaacgttggatttagtttttggaacgtaaattgccaccattgcagtgcagcagggttggcaaaaatttccgcttttgctctctcatactcctcaataattgcatgagatgataacccattaaactgtggatcaattggccccaagttcgactgtttacctaaaacgatctgctcagcacaacaagcaagcattgtgccacaactcattgaaatcataggtacaatcgctcggatattggttccgaactttgaacgaagataatgaccaattgattctagagctgcgatatcgcctccaggagtatggagtaagatatccaatcccagactcgtatctaacccattgatagcagacataagaccatttttatcatcatctgacatctggatcagatgttgaaacccaggcccccctttttgaaggaagcctgagtaataagaaattacatttcggccagtatgtttcgataaatcacgtaagtacttgtggcgaacctcatccgctggtgtacgttgagcgatagtacccatctcacccaatacgtctatccaatttggcatgttatcaatttatcagtatgagtacagttggtgagattgctgaccgttctgctcagtagtatttggtgttactgtgctgtatgaatagagcacaccacttctcacattcagatcgttttgctgagcgagaacacgcatagcaaaatgctgtacggattcgcctttttgaaactcttggggttgtatgcccattttttcgtaaaattcagcagcgctcatgatatgtccctcgtttttttctacatctatgcaattccaggagccatcaacacaagatgtagtagttagcagtcgtcaaatacacgaaaagcctcaagatgaggcttaaaaagattctttttgataaagatttagccaaactatagcggtcaaaatgcagatttgacaagtataaaaagcacttaaagcctataaataggcagtttttgagaattaaagcatctttaatgaggttgaacaaaatgcagtcttgacgctgaacaggactttactggaacgtagagctaaatggttcgatttcatgaaccagttacaaaaaaacccgctcatcggcgggtttataaaactttggcaacatatcaaatatgcttcaaatatggcttattttgttgcattttgcaagcgtgtttgaaggagatggtgaaatttacttcacatttctgccactttgagggcttcttcttcctcatagtattcaagagccatggccaacgcagattcatcaagctgggtaaaagcggcctttaacccagcccagtgccctgaatagacacgcaaccatgtcgaacggtcaacgctaaccatgcgggccaacgctgcaccagcatagtctttataggtttcattatttctggttgcggcaatttcctgccctgccagccataccaggcctatcagtttctttactacgcgctcctgaagggagttatcacccaggcatttctgataagttttccagacgtattcacacatcatcacctggtgcttatagctaaggtcaaaaccgtagcagtaccgcaaccaggcctgctggtatccactaagcgcggacactgccctacgccacggcgcggactcaaattccgcatcttttatcggcggcattggcctgcggcggctgcgtgtttccagcacatacagtggcgcggaaagtgagttaacaaagcgtggccccttctctccttcgagttcgacgagatgaattccacggcgaggggtggcatttttgtctgctggtgggtgttcactgaaagcctcaagctgcccttttgttcccccagacaggtcaggtagcgcgcggcgcaattctattcttacaaaattcaggtcttgttgattcatgcttctttgcgctccatacacttaagctttcgcaattacgccgatcgccagcgcccgatccataaaacgcagtagcagctcaagctgcgtaccatgcttctgctcgaatgccggtacatcggcgtgtaactcgtcgtggcactctctgcacagagggatcacgaagagatcatgggcttttgttgctgtcccccccataccgtgccctacgatatggtgcggatcatctgctggccgtcggcaacactcacagggttgtgttttaacccagcgggtgtacgtctcatttatccagcggcgtcgctttggcctgagcatgaaagattctggagactccggatcaacagagagcgtgaggatcttcttcgccttctcctgcacgaggctggttgcagaagcggaaggcacaatgtcgctttccctcatgaccgagcggatcttatcatccggaaggcgtagccccttgtgcgcaacgctttccggaataacatcagccaggtcgtttctgaccatccaccagcacagttccggaagcgtcaggatatgcgactcgggaaaaccagaatcacgccgaatgacttccagaatccaggataccaggtttcctgccgctatacctgcaagctgttcggtatgctgccccgacaaagtgtgatcgcaatgccagcacaggcgaatacttcctggtgggtgccgcattgttgtgaagttcttgtcgtgccacgatgaatgtggccactggcattcaaaccgattactcaaccattgctcaagggaaggaagcccaccagcacgctgaataacccgatcattctcgaagacctgccgcattaccggatcatcagccagcggctgaatggcggcgggaacagctcctgtactgaatgacgccatttcttctggttcaggctcaagcagaacgcgaccgcgcataaagaggtgcatcagttccgcgccgggacgaaacaacacaatccccatacgatgggcgatctcgggggtaagcagagctctcacgcgacctgccccctggcaatgtgttctgcccacagtccaccaatccagcgcacgcctttcgccgtgaaacgtgcctggctgaatgcatgatttgaggttacggatgtgccggttttcacttcaaaacggcccgcatcaatatgctgatgccgtggggtcatcgttccgccaagacgatacatgatgtcgttctcaaggaggaataaccgcagatcgggctctttggccttaagcagttttgccacctggcggaatgacattgacccactggctgtacagtaccgatcaacaaacgctaccttcggcgccgcggcagccagttcgttagtcaactgctgtttttgttctgcaaggtcagctgcaagacgtagggcttcagagaatgattgaggaatcgtctgctgctgtgcctgctcaagctcctgccagcgatcaaccagacgcgcggtaaactccggcgacagctgcgcgacaacgatataactgtcccgcttccctatcagataaaccgataccgactgattgaggtgatttttaacttcccccattggggggagttcaataacaccgcgctctgccaggcgttcaatggaccgtttaacatggtcatgtcttgattccaccagctcagcaatatcgctgctggacatggttaacgctgttgttgctaactggctcatacttttctccatatcaggcggctgcacccgccggttcatatctgctgattgttatctctacccgacctttcggcacaacgggtccccattccaccagcatgcgcttaatctggctgtcgtcttcccagacacccgcatgcgtcagcgcgtcaaacagggctttgttgtaattatcgatatcccggcggcgcgcatccggcgggtacagagtgatttctaccgctgccagttcagtcgatggcttcgggagacgtcgtaattgctcaatgatcgccacgcaggcagcgctctggtatttacggccagcggcgctaatgaggtgacgaccggccagcggccccttgttaggggcgcgccagtaagtgttcacgctcggaggaaaaggcaggatcagtttcacgcggcctctccccgcatattgcgaacaagttcagaagcagcagtaatgatttcgctggtggcagtccgctccagccagagttgattgatattggctttcagcttgtgttgcagtgactcatccagcatgtcagcaccatccacctggtcgaatacaattctaacctccagcggccagatacgggactcgggaagcggatccgatactggtttagctttctcacgaatgtgcatgcggatctggcgaatattggaccaactggaaacatccaggcttcccatggctgcaatgaagtcagtgctgttcatgccatattcaccggatgcttcaagggcaacagtgcgaatacgttccgacatatccaggcgcacagcagcgtcatcgaattcaatcgacaacagccactcatccacaccgaacaaaatactctcacgaataagcagcttcgctttgtcgatcgttaaaggtgatacctgagtgaattccggtgcttcgacagaatccgccgcccaggtatgcccaaacttcgattcactgaatgtgtattcttctttatcgccaaacgcagctctaacgcatgcccacgcctcgacaccgctgatagcaaaaatatctttctgggtgagtggcaactctgcttctggcttatcagctgcaggaggtgtggcagttacaggttgagacttgctggcagcaaattgtgccaaagccataaacgcccgcccttttgcctccagttctgtgcggttgatatagctgaaccgctcgccacgccatgacttatcgaatacagctatggcaccggcaaaaaacgcgctggtgggtttctgtttttcgtcagcaggtacaaaccacacaggcagatcgaacccaatgcgcccgcgaatgaatacaatgtgatcggcatcttccggccaccacgtttcactcggcgcggcttttatcaggaatacatagcgaccgcccttctcgcgctgggctgctgcgtagttcatgatgtgcgtcatgccggtgatcgcctgcttctcgtggtactgcgaacggctatacggtgggttgccatagccagcgccacccagttcagccagacgttcagaccagtcctgcgtcagcgcgttatcttcggcggtgtaccatgccgggcatttcgcgttgtcgtcgtcagcaaacaaatccagaactaatggaccaaatagcgcgttgatcccccaaaaaagcagatccggtgtccgccactgatcgccaacctctttcaattcgtgagctggtttgctacgcagtgccgccagcgcctggcaatatttattggtcatcatgaacggaaccccgaattttctggcagtgagtaatcaacactctggaagtttgcgcggctggctgagttagtctcccatttgccgttaacgcgttcaggccggccagcactggaccatttggtcgcgctttgcaggtaaccagggaagttttttggaatgaacagagttgccgggcggaggtattgcgcctgctcgctatcacgccaatcggcatttttgtaatccactaccaagcacaggtcatcaacagtgaattgttcccgaagacgggcgcgaatattctccagcgacgtgctgcatacctggtagcgtgagccagtagtctgattcaggtaagacaaaacctgtctggcctgatcagtaatcacaacctcagggtcgggttgcgccgcaaccggacaagagggttttgaagttacttgtggttcttgttttgattttactgacggatccccaccagattctgacgggtcaaaaccgccttttttgctggatttcgacgcctcaaattttgacgggtcggattttgatgcatcagattttgatgggtcagattttgatgcgtcagattctgacaggtgagaaaaagcagcctctcgcaacttaacgacgttaagctgatatacgttcgatgcgttacggttcccattacggcgctgcttacgggaaagccagccatccttctcaagctgagcaagggccgtccttaccgtactttctccagcaccgatttgacgtgcgatcgtgccaatagacggccagctaacaccctcatcactactgaagtcggccagacgcgccatgatggcaacgctggataacttcatgcctgacgaagcgcatgcatcccatacgtaaccggttaatttagtgctcatggtcgtcctttaattctgtaaatttacgctggaattgttcaagagggctgaagcactcatgatcgtacccttcgcgaaggtatataacgcgctgtgtatctggctcccagcggacaactctgacgggaactccgtagtgatctctgaaccgccggttaacttcagccattcctcgcgccccttctcgttcatctgaacaaatgcttctaccatcaagtctgctggctggtagttgcctccatcagccgcgttatttatgatttccacatagccgaactgggcatctttacccaccagcggcaaacatctgaattgcttagctggtctgaatcggtttacactgttcatgcgttagtttctccactgatacgacacgccaaggcgcccggagctgcacactcgcgggcgtcaccttttctgcctgttgaaacgaatacgtcaatcgcctgatctgaaacaccaaccccataaagcgccataaatcccaggaacccgtgaatctggtggcggagcttcttactgaataattctgaaagcattttgcgctctgatgaatcaattaccccatcagccgctgctgccatcttggcattagccagctcaccagatgctgctgccgctttcatctcaatgctgtacagctcaacgttatccaggctctcagcagttggaacatccaccagccatttcccttttcggttcgcctggtactcggccaagtaacaagaaccagacaggtcctccatccgttccagttctgccaaggtaaagaaccgactgccacacttctggtacaggtggttgtggaactggtcgatagtcatccctaaatcggaagccatacctaagcgaccatgcttgtgtgccttacacatcaggcggattgctgtatttatgctgtctaccatgttgatttccctctggtagttaataatcaacttaaagttgactattgttgttagcggaaggtatgccgtcatttttgttcggataaatatcaggtcgtaattgatggggagttactacccatccgccccattggcagagttgaataactctttcagaaggtactcggttctttgcaatccagttcgcaacagattgaactgattggaattcaaaccgccttgatacctctgaaatcgacccgatcgccttcacagctttagctgttacattcttgtgttgagatgacatgtgttctcctatgactaagcctgcatcaatactacttatagtagcaattattagcaacttaaaatagaaatgacaactatgccttgtgcgcttaatcttctacttatggtggaaaatgctaaatacaaagactttgccgaaaggctaaacaggtctctccaagagcaatctattggagttaaagaattgtcagagttcagtggtgtctcgtatgagatggcgcggcgctacactcttggtactgcaaagccgagagatgagaagatgattcgaattgcagaaagacttgccgtctcaccggcttatcttgattatggtgtgcctgttaatggtggcgacgcgccagccaaaggcacggtcagaatagagcaattggatgttcatgcttcagccggttccggatatataaaccaaccattccctacaatagtgagctcaatagagattccagaagagaggatcttcgagttgtttggtcgtagaagccttgatggcatcgtcatgataaatgttgatggcgatagcatgatgcccacgctttgcccaaaggacctgcttttcatagacagcaaggttgaacaattcagcggcgacggcgtttatgtgttcaattttgaagacagtacgttcgttaaacgtttgcagaaggtaaaagggcgccgactggcagttctttcagacaatgaacattacccgcccttcttcatagaggagcatgaaatgaatgaactatacatattcggcaagctaatcagatgcttacctctaaaaatgatagagtttggctaataattaattcatcaagaaaccggcgaaagccggttttttttacgcctccaattcctcacctcataacactacactactaaaaatttcattttctactttttgttgttgcaattatctacttaaagtagctatagtcattgcatcgaaagcgaacaggcaggacgcccacgaagtagccgccggtggcatatgaataaccggatgattcgctgacagaaaacttaggttgggggtagaggtttacatgaatcatttattcacatgctcattttgcggagcaaccgaactgggagcgataaagatcgtcgcaaaaggtggtaaggacgaacctgccatctgttcggaatgcgtagtcacatgtgtagaaaaaatgatcctgactaaaaaatcagaggctgaaaaaccaacctctgataacgaaataatatcagtcgataaaaaactatttaaagagcttcttcagcttgtcctcaaccttcctgatttcggaagtaagctggctgctgttgacattgatagtagctccacatcgacaagtgaaacttttgttcgacttgagccaagcgattttcttcttcgtcttagtgccgcacttagggcatgcgggtaacgtaatttcctggttatcaaaagcgcccataaacatccctcttggttgtgtgagaacaccaagataccaccgcgcctgatgtggttaaaagcaggctaaagcaataacaagtaactccctgttctggcggcccggtgttttcccgtgtatttccggtaaccgccagcctttttcagggcacaacagaaaagggcatcaccgggcgacgggctcataacccaatccacccgggcaaaaagaaagcggtctctgcaagccgccgaccaatgcaggtgcccttctctgttgtgtatggagaaactaactttttagcgtctgtgcagatgcgctgaggaaccgagaatgaataatccgtttttcaaaaatatgttggtgtatcgcattagtcgcgatttcaccatcaaccaggaagagctggaacagcagcttgaactatttcgcttcactccatgcggtagccaggatatggcaaaaaccggttgggtatcaccacttggtcagctgtcagatcgcttgcatcacactgtcaataatcaagtgttgttggttattcgccgggaagaaaaaatactgccatctcctgtcattactgaagaactgcgcaagcgtgtgtcgcgtctagaatccgatcaggggcgtcgcctcaaaaaaactgagaaagattcgctgcgtgatgaagtgttgcactccctgcttcctcgggcgttctccaaaaactcgactgttggtttgtggatcaacgtcaccgacggtctgatcatggttgatgcagccagcgctaaacgtgccgaagactcactggccctgcttcgtaaaactctcggttctctcccggtggtaccgctgactatggaaacgccgatcgaactaactatgaccgactgggttcgttccggtagtgcgcctgctggctttggcctgggtgatgaagccgaactgaaagctattcttgaagatggcggtattggacgctttaaaaaacagactctggtcagtgacgaaattcatgtgcatctggaagctggcaaagtagttacaaagctgtctatcgactggcaacagcgcattcagttcgttctttgcgatgacggcagcatcaaacgccttaagttctctaatgagattacagaacaaaacgacgatatcgaccgtgaggatgcggctcagcggttcgacgctgactttgttctgatgaccggcgagcttatctctctcattaacggattaacaacctctctcggcggcgaagccaagcgataaacaccaggcaacaattacccccataagcatgggttgggttgctgcacgctaaattcagcaattcattaatttaatggcgcggtgcagcgcgccaatatggagaaaaccatgagctacattcagacattatccggcaaacattttaattacctcgatatccaacaggacgatatcgtgatcgaggatattgctaccgcgttgtctcatatctgccgctttgcagggcatcttcctgagttttacagtgtcggccagcatagcgttttaaccagccacctcgttccgcaggagtttgcattagaagcactgcttcatgatgctgctgaagcctacctgcaggacatcccctccccacttaagcgcctgcttccggattaccaggcaatcgaagctcgtgtggacgcagccattcggcagaagttcggtctaccaactgagcaacacccaaccgtgaaatatgccgacctggtgatgctcgccagcgaacgccgcgattttgagattgacgaaggttccatttggccatgcctcgagggagttgtcccaacggatttattcattatcaacccagttcgtcctggccagtcatacggcatgttcatcaatcgctttaacgagttgatggagcagcgccaatgcgccgcatgaaggtaaaagaactcgtagcggaggcgtttgcctccgttgctgaattgccaccaaagcatgcgccgcttatgcgcgaagtcgccaccagactggacgctacgttcgcagcattaaaagagtctctggtgcaactggaacaggaacgtaaagataaaacgccatgaccgtatttgaatatctccaggctcatccgaataccaccagcggtgaaatcgccaaaggtatgaacaaaaagaccccagcggtcgccggagcattatctcagctctatggcaccggtcggatcgtgaagtctggtgttcgcaagggtattccaacataccgcattaacgatatgccgtttggttgcagtaacagcctaaccatgatgtttaaccagctcttgagcagagccagacaaggagcagcccaatgacagcactcaacaaacaggcgctgcgtgaagaattccagttcatgcaggacaactatagcgacccggcagaccacgatcggcaggtgatttacatcgaggcggaggcgctgctggatgagttggaagccaaagactcaacgatagcagcacaacaacatgagatccgtatgttgctgaatgcgcttgaggaaaaaccatgcccgaaatgcaacgacacaggaatgactgatagtggcggcacgcagccatggggcgagccgattgagattgaatgcgactgccgacagcaggatgccaacaccgcagaacttgtagccgctggcattggcgtgaagggggagtgagatggataaattaatcaaacctaccgccaaaggtaaatatgacggttcatgtgattatctttgctcggaagatgcgcgattcatcgttatgcgcggcgattatacggaagcggaaataattcaggcttctgtgtctcaagatgtaatcgactcggatggtgcggctgattttgcaagtagcgcccgctattatcagtgctggtacaaagttagcccaataggtggtcaggatggctattcaggctggcatcatcctcgtgattcgccgtgtcgcggtgcatatttcgcatcagttttgcaatgggattaaggaggactaacccatgacaactaacaaccacccggcgcacggtcctgtatcactcgatcgcctgcaccagatacgcgaacacctgctgcatgatacccaatactcaaacggcgggaacagagcctacattctcgctgatgtattgaaggtgattgatggggctattgcccgcgagctggtacgccgtgagcatgcagcgtggtcacaggctactttcggcgatgtcggtccagttggtccgctgaagcacctttccaaagaagcgctcgaggctgctgctgaaccaggcgaccttagcgaatgggctgacatgcaattcctgttatgggatgcgcaacgtcgtgccggtatcagtgatgagcagattacccaggcaatgataaaaaagctggctataaataaggttcgccaatggcctgagccgaaagacggggaacctcgattgcatatcaaagaacagtcagagcaggagaaaaaataagaatgtttagcctgattcggcgcggtcaaatctacacggacagtagcaactggcccgtaattatccatagctgtagtgatcactcggtccgaattaaacgcaatgatggcgagctgagaacgattagcatcaaacgctttaacgaagattttgaacgagtggagcatgatgagtatcgcaaaatatgtgccgaaatagagcaggaaacaaacctgaaaaacctacgtgcgatgcgtcgcggcaagattactgaatagccaaacaggagaatatttaacgtgaacaacttaatgatcgaccttgagtccatgggcaaaaaaccgaatgcccctattgtctccattggtgccgtattcttcgatccgcaaagcggtgaactgggtcaggagttttacaccgctgttaatcttgaaagcgctatggagcagggagcggtgccggatggtgacactattctgtggtggttaagacaaagctcagaagcacgatcagcaatctgtgttgatgatgcgatgccgatatcatctgccctatctgaactgagccatttcattaatcggcattctgataaccctaaatatttaaaagtttggggcaatggagctactttcgacaacgttatattgcgcggcgcatatgagcgtgccggccaggtttgcccgtggcaattttggaatgatcacgacgtcagaaccatcgtcacattaggcagatctgtaggtttcgatcctaagcgtgatatgccatttgatggggttgcacataacgcactggctgatgcccgccaccaggcgaaatatgtttcagcgatttggcagaaactaatcccaaccaccagcaacagctaaagttttccccgggtgcagccgggataatggagaaataactatgagcaatattttccagttagctcccaacgattgggtttgtgaaagcgttttgatcgcggttactgggctcaaacccggaaccatcctccgtgccagaaaagaatgctggatgattgggagggagtatatccacgtatcgcctgacggaaatcctaaaccttccagtgagtgcatgtataacagaaaggctgtagatgcctgggtcgcttcaatgaaaagcaagcaaccagggtgatttgatgccatgaaaaaggtaagctcgtatcgctcttgggcgtctggaggtaacaccaatggataaagtcacatatccaacaggcgtcgaaaaccacggtggcacattacgcatctggtttaattttaaaggtaagcgtgtcagggaaagtctcggtgtccctgacaccgctaagaacaggaagatagccggggaactgcggacatcagtatgttttgccatccgcacaggaacctttgattatgcaacccagtttcctgactcccctaacctcaaggcttttggtgtaagtaaaaaagacattacagtgaaagaacttgaagaaaaatggctggatctgaaacggatggaaatctgcgcgaacgcatttaatcgctatgaatctgtcgcaaggaatatggtgccgaggatcggaggtaatcgcctggtgtcagcagtaaccaaagaggaattgctgtatctgaggaaatatttgctaactggttatcagaatccgacgaaaaacaaagccccggcaaaagggcgaagcgttgttactgtgaactattacatgacgacaatggccggaatgtttcagtttgctgcggatcacggttacttagaggtgaacccattcgagggaattaagcctctgaaaaaagccagggcagaaccagatcctctgtctcgtgatgaatttattcgcctgatagatgcatgccggcatcagcagacgaaaaacctgtggtcattagcagtgtacacaggaatgcgtcacggggaactggtctccctggcctgggaagatatcgacctgaaggctggaacaattaccgtcagacgtaattatacgaaacttggtgagttcactctaccgaaaaccgaggcaagcacagatcgagtggtgcatcttatccagcccgcaatcagtatcctgaaaaatcaggctgaaatgacaaggctgggcaggcaatatcacattgaagtgcagttacgtgagtacggccgttcggtgaaccatgagtgtacattcgtctttaatccgcatgtggtcagacgcagtaagcaggtcggatttatctaccgggtcgattcagtaggcgactcatgggaagcggcacttaagcgtgcggggatcagacacagaaaggcgtaccagtcacgacacacctatgcgtgctggtcattatcagctggtgcaaaccctagttttattgccagtcagatggggcatgcgagcgcgcagatggtgttcaatgtttacggtgcatggatggctgacagcagcgcagagcagatcgcaatgctgaatcagaagctggcagattttgccccattgatgccccatagccacgagaacagtacgggaggattattaaaatcagtaagttaacccctaacgcccgtcatgttaactgtgtggagggtaacaccacgctttatgccctgccgaaacccgaggttgtcctgcgctggcgtgagcagaccacagatgacttccgcttctgttttaagtttccggcgaccatttcgcatcaggcagcattacggcattgcgatgatttagtgactgaatttttgacccgcatgtcaccgttggctccgcgcattggacaatactggctgcaactgcctgccacattcggcccacgggagctgcctgcgctttggcattttctcgattctcttcccggtgaatttaattatggggtggaagtccgccatccacagtttttcgccaaaggggaagaggaacaaacgcttaatcgcggtttacatcagcgcggcgttaatcgggtgattttagacagccgcccggttcatgcagcacgtccatacagtgaagctattcgcgacgctcaacgaaaaaaacctaaagttccggtacatgctgtactgacggcgaaaaatccactgatccgttttatcggtagtgatgatatgacgcaaaaccgggaattatttcaggtctggttacaaaaattagcgcagtggcatcagaccactacgccttatctttttttacatacgccagatattgcccaggccccggaactggtacataccctgtgggaagacttacgtaaaacgcttccagagatcggagcagttccggctattccacagcaatcttctcttttctgaatttgccacctatcatagacaggtgccatcggccattttaaagggagtttgtatggtaagcgcgctgtatgccgttttaagtgcgttgttattaatgaagttctcttttgatgtcgttcgcctgcgaatgcagtaccgcgttgcctatggcgacggcggttttagcgaactgcaaagcgctattcgcattcatggtaacgcggtggaatatattcctatcgcgattgtgttgatgctgtttatggaaatgaatggcgcagaaacctggatggtgcatatttgcggcatcgttttgcttgctggtcgtctgatgcattattacggttttcatcaccgtctgttccgctggcgacgttctggcatgagcgccacctggtgtgcgctgttgctgatggtgctggcgaatctttggtatatgccctgggagttggttttctccctgcgttagcgcacaatacgccactttctttttcccggatttttacgttatgtctcaccgcgacacgctattttctgcccctatcgccagactgggcgactggacctttgatgaacgggtagctgaagtcttcccggatatgatccagcgttccgttcccggctattccaatattatttccatgattggtatgttagccgagcgcttcgttcaacctggtacgcaggtttacgatctgggttgttctctgggcgcggcgacgctctcggtgcgtcgcaacattcatcatgataattgcaaaattattgccatcgacaactccccggcgatgattgaacgctgccgtcgtcatattgacgcctataaagcccctacgccagtagacgttattgaaggtgatattcgcgatatcgccattgaaaacgcatcgatggtggtgctgaattttaccctgcaattcctggaaccttccgagcgccaggcgttactggataaaatttatcaagggctgaacccgggcggtgcgctggtgctttcggaaaaattcagtttcgaagatgccaaagttggtgaactgctgttcaacatgcaccacgactttaaacgtgccaacggttacagcgaactggagatcagccagaaacgcagcatgctggaaaacgtgatgctgaccgattccgtggaaacccataaagcacgcctgcataaagccggttttgagcatagcgagctgtggttccagtgctttaactttggttcactggtggcattaaaagcagaggacgctgcatgatcgactttggtaacttttattctctgattgccaaaaatcatctttcacactggctcgaaacgctgcccgcgcagattgctaactggcagcgcgagcagcagcacgggctgtttaagcagtggtccaacgcggtggaatttctgcctgaaattaaaccgtatcgtctggatttattgcatagcgtaaccgccgaaagcgaagagccactgagcgccgggcaaattaagcgcattgaaacgctgatgcgcaacctgatgccgtggcgcaaagggccgttctcactgtatggcgtcaacatcgataccgaatggcgttccgactggaaatgggatcgcgttatgccccatctttctgatttaaccgggcgcaccattcttgatgtcggctgtggcagcggttatcacatgtggcgcatgattggcgcaggggcgcatctggcggtgggtatcgatcccacgcagctattcctctgccagtttgaagcagtgcgtaaactgctgggtaacgatcagcgcgcacatttgttaccgttaggtattgaacaacttccggcactgaaagcctttgataccgtcttttcgatgggcgtgctttatcatcgtcgttcaccgctggagcatctctggcagttaaaagaccaactggtgaatgaaggcgaactggtgctggaaacgctggttattgatggcgacgaaaacacggtgctggtgccgggcgatcgttacgctcaaatgcgtaatgtctatttcattccttccgcgctggcgctgaaaaactggctgaagaagtgtggttttgttgatattcgcattgcagatgtgagcgttaccaccacagaagagcagcgacgcaccgaatggatggtcaccgagtctctggccgattttctcgacccgcatgatccgggtaaaacggtggaaggttatcctgcgcctaaacgcgcggtgctgattgcgcgcaagccgtaaaggtctggtaatactgccggatgcggcgtgaacgccttatccggcctacaaagtcttgctaattcaatatattgcaggggctatgtaggcctgataagcatagcgcatcaggca

SEQ ID NO: 415-499 - skipped (85 entries)

SEQ ID NO: 500 - protein, 532 aa, Photorhabdus luminescens
  REGION 1..532: note = misc_feature - Photorhabdus luminescens PAL3 Wild-type
MKAKDVQPTIIINKNGLISLEDIYDIAIKQKKVEISTEITELLTHGREKLEEKLNSGEVIYGINTGFGGNANLVVPFEKIAEHQQNLLTFLSAGTGDYMSKPCIKASQFTMLLSVCKGWSATRPIVAQAIVDHINHDIVPLVPRYGSVGASGDLIPLSYIARALCGIGKVYYMGAEIDAAEAIKRAGLTPLSLKAKEGLALINGTRVMSGISAITVIKLEKLFKASISAIALAVEALLASHEHYDARIQQVKNHPGQNAVASALRNLLAGSTQVNLLSGVKEQANKACRHQEITQLNDTLQEVYSIRCAPQVLGIVPESLATARKILEREVISANDNPLIDPENGDVLHGGNFMGQYVARTMDALKLDIALIANHLHAIVALMMDNRFSRGLPNSLSPTPGMYQGFKGVQLSQTALVAAIRHDCAASGIHTLATEQYNQDIVSLGLHAAQDVLEMEQKLRNIVSMTILVVCQAIHLRGNISEIAPETAKFYHAVREISSPLITDRALDEDIIRIADAIINDQLPLPEIMLEE

SEQ ID NO: 501 - protein, 532 aa, synthetic construct
  REGION 1..532: note = Synthetic: mPAL1
MKAKDVQPTIIINKNGLISLEDIYDIAIKQKKVEISTEITELLTHGREKLEEKLNSGEVIYGINTGFGGNANLVVPFEKIAEHQQNLLTFLGAGTGDYMSKPCIKASQFTMLLSVCKGWSATRPIVAQAIVDMINHDIVPLVPRYGSVGASGDLIPLSYIARALCGKGKVYYMGAEIDAAEAIKRAGLTPLSLKAKEGLALINGTRVMSGISAITVIKLEKLFKASISAIALAVEALLASHEHYDARIQQVKNHPGQNAVASALRNLLAGSTQVNLLSGVKEQANKACRHQEITQLNDTLQEVYSIRCAPQVLGIVPESLATARKILEREVISANDNPLIDPENGDVLHGGNFMGQYVARTMDALKLDIALIANHLHAIVALMMDNRFSRGLPNSLSPTPGMYQGFKGVQLSQTALVAAIRHDCAASGIHTIATEQYNQDIVSLGLHAAQDVLEMEQKLRNIVSMTILVACQAIHLRGNISEIAPETAKFYHAVREISSPLITDRALDEDIIRIADAIINDQLPLPEIMLEE

SEQ ID NO: 502 - protein, 532 aa, synthetic construct
  REGION 1..532: note = Synthetic: mPAL2
MKAKDVQPTIIINKNGLISLEDIYDIAIKQKKVEISTEITELLTHGREKLEEKLNSGEVIYGINTGFGGNANLVVPFEKIAEHQQNLLTFLGAGTGDYMSKPCIKASQFTMLLSVCKGWSATRPIVAQAIVDFINHDIVPLVPRYGSVGASGDLIPLSYIARALCGIGKVYYMGAEIDAAEAIKRAGLTPLSLKAKEGLALINGTRVMSGISAITVIKLEKLFKASISAIALAVEALLASHEHYDARIQQVKNHPGQNAVASALRNLLAGSTQVNLLSGVKEQANKACRHQEITQLNDTLQEVYSIRCAPQVLGIVPESLATARKILEREVISANDNPLIDPENGDVLHGGNFMGQYVARTMDALKLDIALIANHLHAIVALMMDNRFSRGLPNSLSPTPGMYQGFKGVQLSQTALVAAIRHDCAASGIHTLSTEQYNQDIVSLGLHAAQDVLEMEQKLRNIVSMTILVACQAIHLRGNISEIAPETAKFYHAVREISSPLITDRALDEDIIRIADAIINDQLPLPEIMLEE

SEQ ID NO: 503 - protein, 532 aa, synthetic construct
  REGION 1..532: note = Synthetic: mPAL3
MKAKDVQPTIIINKNGLISLEDIYDIAIKQKKVEISTEITELLTHGREKLEEKLNSGEVIYGINTGFGGNANLVVPFEKIAEHQQNLLTFLGAGTGDYMSKPCIKASQFTMLLSVCKGWSATRPIVAQAIVDFINHDIVPLVPRYGSVGASGDLIPLSYIARALCGIGKVYYMGAEIDAAEAIKRAGLTPLSLKAKEGLALINGTRVMSGISAITVIKLEKLFKASISAIALAVEALLASHEHYDARIQQVKNHPGQNAVASTLRNLLAGSTQVNLLSGVKEQANKACRHQEITQLNDTLQEVYSIRCAPQVLGIVPESLATARKILEREVISANDNPLIDPENGDVLHGGNFMGQYVARTMDALKLDIALIANHLHAIVALMMDNRFSRGLPNSLSPTPGMYQGFKGVQLSQTALVAAIRHDCAASGIHTLATEQYNQDIVSLGLHAAQDVLEMEQKLRNIVSMTILVACQAIHLRGNISEIAPETAKFYHAVREISSPLITDRALDEDIIRIADAIINDQLPLPEIMLEE

SEQ ID NO: 504-505 - skipped (2 entries)

SEQ ID NO: 506 - DNA, 2753 bp, synthetic construct
  misc_feature 1..2753: note = Synthetic: pheP integration construct
  source 1..2753: mol_type = other DNA
tcactgcccgctttccagtcgggaaacctgtcgtgccagctgcattaatgaatcggccaacgcgcggggagaggcggtttgcgtattgggcgccagggtggtttttcttttcaccagtgagactggcaacagctgattgcccttcaccgcctggccctgagagagttgcagcaagcggtccacgctggtttgccccagcaggcgaaaatcctgtttgatggtggttaacggcgggatataacatgagctatcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcctcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcatattcaccaccctgaattgactctcttccgggcgctatcatgccataccgcgaaaggttttgcgccattcgatggcgcgccgcttcgtcaggccacatagctttcttgttctgatcggaacgatcgttggctgtgttgacaattaatcatcggctcgtataatgtgtggaattgtgagcgctcacaattagctgtcaccggatgtgctttccggtctgatgagtccgtgaggacgaaacagcctctacaaataattttgtttaatagcttcggtaagtttacggaggatttgtcatgaaaaacgctagtacggtaagtgaggatacagcctcgaaccaagaaccaacgttgcaccgtgggttgcacaaccgtcacatccagcttatcgcacttggcggcgctattggaacggggttattccttggaattggaccggccattcaaatggccgggccagcggttttgttggggtatggtgtagcggggatcattgcatttcttattatgcgtcagttgggagaaatggtggttgaagaacctgtatccggcagcttcgcgcatttcgcgtacaagtattggggtccatttgctggctttctgagtggctggaattattgggtgatgtttgtcctggttggaatggcggaacttactgcggccggcatttatatgcagtactggtttcctgatgtgcctacgtggatctgggccgcggctttctttattattatcaatgcagtcaatctggtcaacgtgcgcttgtatggtgagacggagttctggttcgcattaattaaggtattagctattatcggaatgattggctttgggttatggttgctgtttagcgggcacggcggtgagaaagcctctatcgataacctttggcgctacggtgggttctttgctacaggatggaacgggttaatcttgagtcttgcggtcatcatgttcagttttggtggccttgaattgattggtatcacggcagcagaggcgcgtgacccagaaaaaagcatccccaaagccgttaatcaggtggtgtaccgcatcttattattttacattggttcactggtcgtgttgttggctctgtacccatgggttgaggtgaaatctaactcatcccccttcgtcatgatctttcataaccttgattcaaatgtggtcgccagcgcgttaaactttgtaatcctggtggcaagcctttccgtgtacaattcaggggtctattctaatagtcgtatgttgttcgggctttcggtccaaggaaacgcgccgaaattcctgacacgcgttagtcgtcgtggtgtgcccattaatagcctgatgctgagtggtgcaatcacttctttagtcgtgcttattaactatttactgcctcagaaggcattcgggttattaatggctttagttgtcgcaacgttattgttaaactggatcatgatctgtttagcacacctgcgtttccgtgcggctatgcgtcgccagggtcgtgaaacccagttcaaggccttactttatccctttggtaattacttgtgcattgcatttttaggcatgattttactgctgatgtgtactatggatgatatgcgcctgtccgcaatccttttacccgtctggattgtttttctttttatggcattcaaaacacttcgtcgcaagtaa

SEQ ID NO: 507 - DNA, 2975 bp, synthetic construct
  misc_feature 1..2975: note = Synthetic: mmPAL1 integration construct
  source 1..2975: mol_type = other DNA
tcactgcccgctttccagtcgggaaacctgtcgtgccagctgcattaatgaatcggccaacgcgcggggagaggcggtttgcgtattgggcgccagggtggtttttcttttcaccagtgagactggcaacagctgattgcccttcaccgcctggccctgagagagttgcagcaagcggtccacgctggtttgccccagcaggcgaaaatcctgtttgatggtggttaacggcgggatataacatgagctatcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcctcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcatattcaccaccctgaattgactctcttccgggcgctatcatgccataccgcgaaaggttttgcgccattcgatggcgcgccgcttcgtcaggccacatagctttcttgttctgatcggaacgatcgttggctgtgttgacaattaatcatcggctcgtataatgtgtggaattgtgagcgctcacaattagctgtcaccggatgtgctttccggtctgatgagtccgtgaggacgaaacagcctctacaaataattttgtttaatagcttcggtaagtttacggaggatttgtcatgaaagctaaagatgttcagccaaccattattattaataaaaatggccttatctctttggaagatatctatgacattgcgataaaacaaaaaaaagtagaaatatcaacggagatcactgaacttttgacgcatggtcgtgaaaaattagaggaaaaattaaattcaggagaggttatatatggaatcaatacaggatttggagggaatgccaatttagttgtgccatttgagaaaatcgcagagcatcagcaaaatctgttaacttttcttggcgctggtactggggactatatgtccaaaccttgtattaaagcgtcacaatttactatgttactttctgtttgcaaaggttggtctgcaaccagaccaattgtcgctcaagcaattgttgatatgattaatcatgacattgttcctctggttcctcgctatggctcagtgggtgcaagcggtgatttaattcctttatcttatattgcacgagcattatgtggtaagggcaaagtttattatatgggcgcagaaattgacgctgctgaagcaattaaacgtgcagggttgacaccattatcgttaaaagccaaagaaggtcttgctctgattaacggcacccgggtaatgtcaggaatcagtgcaatcaccgtcattaaactggaaaaactatttaaagcctcaatttctgcgattgcccttgctgttgaagcattacttgcatctcatgaacattatgatgcccggattcaacaagtaaaaaatcatcctggtcaaaacgcggtggcaagtgcattgcgtaatttattggcaggttcaacgcaggttaatctattatctggggttaaagaacaagccaataaagcttgtcgtcatcaagaaattacccaactaaatgataccttacaggaagtttattcaattcgctgtgcaccacaagtattaggtatagtgccagaatctttagctaccgctcggaaaatattggaacgggaagttatctcagctaatgataatccattgatagatccagaaaatggcgatgttctacacggtggaaattttatggggcaatatgtcgcccgaacaatggatgcattaaaactggatattgctttaattgccaatcatcttcacgccattgtggctcttatgatggataaccgtttctctcgtggattacctaattcactgagtccgacacccggcatgtatcaaggttttaaaggcgtccaactttctcaaaccgctttagttgctgcaattcgccatgattgtgctgcatcaggtattcataccattgccacagaacaatacaatcaagatattgtcagtttaggtctgcatgccgctcaagatgttttagagatggagcagaaattacgcaatattgtttcaatgacaattctggtagcctgtcaggccattcatcttcgcggcaatattagtgaaattgcgcctgaaactgctaaattttaccatgcagtacgcgaaatcagttctcctttgatcactgatcgtgcgttggatgaagatataatccgcattgcggatgcaattattaatgatcaacttcctctgccagaaatcatgctggaagaataa

SEQ ID NO: 508 - DNA, 4637 bp, synthetic construct
  misc_feature 1..4637: note = Synthetic: mPAL1-mPAL1 integration construct
  source 1..4637: mol_type = other DNA
tcactgcccgctttccagtcgggaaacctgtcgtgccagctgcattaatgaatcggccaacgcgcggggagaggcggtttgcgtattgggcgccagggtggtttttcttttcaccagtgagactggcaacagctgattgcccttcaccgcctggccctgagagagttgcagcaagcggtccacgctggtttgccccagcaggcgaaaatcctgtttgatggtggttaacggcgggatataacatgagctatcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcctcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcatattcaccaccctgaattgactctcttccgggcgctatcatgccataccgcgaaaggttttgcgccattcgatggcgcgccgcttcgtcaggccacatagctttcttgttctgatcggaacgatcgttggctgtgttgacaattaatcatcggctcgtataatgtgtggaattgtgagcgctcacaattagctgtcaccggatgtgctttccggtctgatgagtccgtgaggacgaaacagcctctacaaataattttgtttaatagcttcggtaagtttacggaggatttgtcatgaaagctaaagatgttcagccaaccattattattaataaaaatggccttatctctttggaagatatctatgacattgcgataaaacaaaaaaaagtagaaatatcaacggagatcactgaacttttgacgcatggtcgtgaaaaattagaggaaaaattaaattcaggagaggttatatatggaatcaatacaggatttggagggaatgccaatttagttgtgccatttgagaaaatcgcagagcatcagcaaaatctgttaacttttcttggcgctggtactggggactatatgtccaaaccttgtattaaagcgtcacaatttactatgttactttctgtttgcaaaggttggtctgcaaccagaccaattgtcgctcaagcaattgttgatatgattaatcatgacattgttcctctggttcctcgctatggctcagtgggtgcaagcggtgatttaattcctttatcttatattgcacgagcattatgtggtaagggcaaagtttattatatgggcgcagaaattgacgctgctgaagcaattaaacgtgcagggttgacaccattatcgttaaaagccaaagaaggtcttgctctgattaacggcacccgggtaatgtcaggaatcagtgcaatcaccgtcattaaactggaaaaactatttaaagcctcaatttctgcgattgcccttgctgttgaagcattacttgcatctcatgaacattatgatgcccggattcaacaagtaaaaaatcatcctggtcaaaacgcggtggcaagtgcattgcgtaatttattggcaggttcaacgcaggttaatctattatctggggttaaagaacaagccaataaagcttgtcgtcatcaagaaattacccaactaaatgataccttacaggaagtttattcaattcgctgtgcaccacaagtattaggtatagtgccagaatctttagctaccgctcggaaaatattggaacgggaagttatctcagctaatgataatccattgatagatccagaaaatggcgatgttctacacggtggaaattttatggggcaatatgtcgcccgaacaatggatgcattaaaactggatattgctttaattgccaatcatcttcacgccattgtggctcttatgatggataaccgtttctctcgtggattacctaattcactgagtccgacacccggcatgtatcaaggttttaaaggcgtccaactttctcaaaccgctttagttgctgcaattcgccatgattgtgctgcatcaggtattcataccattgccacagaacaatacaatcaagatattgtcagtttaggtctgcatgccgctcaagatgttttagagatggagcagaaattacgcaatattgtttcaatgacaattctggtagcctgtcaggccattcatcttcgcggcaatattagtgaaattgcgcctgaaactgctaaattttaccatgcagtacgcgaaatcagttctcctttgatcactgatcgtgcgttggatgaagatataatccgcattgcggatgcaattattaatgatcaacttcctctgccagaaatcatgctggaagaataaaacaacacccactaagataactctagaaataattttgtttaactttaagaaggagatatacatatgaaagctaaagatgttcagccaaccattattattaataaaaatggccttatctctttggaagatatctatgacattgcgataaaacaaaaaaaagtagaaatatcaacggagatcactgaacttttgacgcatggtcgtgaaaaattagaggaaaaattaaattcaggagaggttatatatggaatcaatacaggatttggagggaatgccaatttagttgtgccatttgagaaaatcgcagagcatcagcaaaatctgttaacttttcttggcgctggtactggggactatatgtccaaaccttgtattaaagcgtcacaatttactatgttactttctgtttgcaaaggttggtctgcaaccagaccaattgtcgctcaagcaattgttgatatgattaatcatgacattgttcctctggttcctcgctatggctcagtgggtgcaagcggtgatttaattcctttatcttatattgcacgagcattatgtggtaagggcaaagtttattatatgggcgcagaaattgacgctgctgaagcaattaaacgtgcagggttgacaccattatcgttaaaagccaaagaaggtcttgctctgattaacggcacccgggtaatgtcaggaatcagtgcaatcaccgtcattaaactggaaaaactatttaaagcctcaatttctgcgattgcccttgctgttgaagcattacttgcatctcatgaacattatgatgcccggattcaacaagtaaaaaatcatcctggtcaaaacgcggtggcaagtgcattgcgtaatttattggcaggttcaacgcaggttaatctattatctggggttaaagaacaagccaataaagcttgtcgtcatcaagaaattacccaactaaatgataccttacaggaagtttattcaattcgctgtgcaccacaagtattaggtatagtgccagaatctttagctaccgctcggaaaatattggaacgggaagttatctcagctaatgataatccattgatagatccagaaaatggcgatgttctacacggtggaaattttatggggcaatatgtcgcccgaacaatggatgcattaaaactggatattgctttaattgccaatcatcttcacgccattgtggctcttatgatggataaccgtttctctcgtggattacctaattcactgagtccgacacccggcatgtatcaaggttttaaaggcgtccaactttctcaaaccgctttagttgctgcaattcgccatgattgtgctgcatcaggtattcataccattgccacagaacaatacaatcaagatattgtcagtttaggtctgcatgccgctcaagatgttttagagatggagcagaaattacgcaatattgtttcaatgacaattctggtagcctgtcaggccattcatcttcgcggcaatattagtgaaattgcgcctgaaactgctaaattttaccatgcagtacgcgaaatcagttctcctttgatcactgatcgtgcgttggatgaagatataatccgcattgcggatgcaattattaatgatcaacttcctctgccagaaatcatgctggaagaataa

SEQ ID NO: 509 - protein, 458 aa, Escherichia coli
  REGION 1..458: note = misc_feature - PheP E. coli
MKNASTVSEDTASNQEPTLHRGLHNRHIQLIALGGAIGTGLFLGIGPAIQMAGPAVLLGYGVAGIIAFLIMRQLGEMVVEEPVSGSFAHFAYKYWGPFAGFLSGWNYWVMFVLVGMAELTAAGIYMQYWFPDVPTWIWAAAFFIIINAVNLVNVRLYGETEFWFALIKVLAIIGMIGFGLWLLFSGHGGEKASIDNLWRYGGFFATGWNGLILSLAVIMFSFGGLELIGITAAEARDPEKSIPKAVNQVVYRILLFYIGSLVVLLALYPWVEVKSNSSPFVMIFHNLDSNVVASALNFVILVASLSVYNSGVYSNSRMLFGLSVQGNAPKFLTRVSRRGVPINSLMLSGAITSLVVLINYLLPQKAFGLLMALVVATLLLNWIMICLAHLRFRAAMRRQGRETQFKALLYPFGNYLCIAFLGMILLLMCTMDDMRLSAILLPVWIVFLFMAFKTLRRK

SEQ ID NO: 510 - protein, 473 aa, Proteus mirabilis
  REGION 1..473: note = misc_feature - LAAD Proteus mirabilis
MNISRRKLLLGVGAAGVLAGGAALVPMVRRDGKFVEAKSRASFVEGTQGALPKEADVVIIGAGIQGIMTAINLAERGMSVTILEKGQIAGEQSGRAYSQIISYQTSPEIFPLHHYGKILWRGMNEKIGADTSYRTQGRVEALADEKALDKAQAWIKTAKEAAGFDTPLNTRIIKGEELSNRLVGAQTPWTVAAFEEDSGSVDPETGTPALARYAKQIGVKIYTNCAVRGIETAGGKISDVVSEKGAIKTSQVVLAGGIWSRLFMGNMGIDIPTLNVYLSQQRVSGVPGAPRGNVHLPNGIHFREQADGTYAVAPRIFTSSIVKDSFLLGPKFMHLLGGGELPLEFSIGEDLFNSFKMPTSWNLDEKTPFEQFRVATATQNTQHLDAVFQRMKTEFPVFEKSEVVERWGAVVSPTFDELPIISEVKEYPGLVINTATVWGMTEGPAAGEVTADIVMGKKPVIDPTPFSLDRFKK